(19) United States
(12) Patent Application Publication
(10) Pub. No.: US 2021/0015866 A1
(43) Pub. Date: Jan. 21, 2021

(54) TISSUE RESIDENT MEMORY CELL PROFILES, AND USES THEREOF

(71) Applicants: La Jolla Institute for Allergy and Immunology, La Jolla, CA (US); University of Southampton, Southampton Hampshire (GB)

(72) Inventors: Pandurangan VIJAYANAND, La Jolla, CA (US); Christian OTTENSMEIER, Southampton Hampshire (GB); James CLARKE, La Jolla, CA (US); Tilman SANCHEZ-ELSNER, Southampton Hampshire (GB); Simon ESCHWEILER, La Jolla, CA (US); Ferhat AY, La Jolla, CA (US)

(73) Assignees: La Jolla Institute for Allergy and Immunology, La Jolla, CA (US); University of Southampton, Southampton Hampshire (GB)

(21) Appl. No.: 17/040,429

(22) PCT Filed: Mar. 22, 2019
(86) PCT No.: PCT/US2019/023767

§ 371 Date: Sep. 22, 2020

Related U.S. Application Data
(60) Provisional application No. 62/770,412, filed on Nov. 21, 2018.
(60) Provisional application No. 62/647,588, filed on Mar. 23, 2018.

(51) Int. Cl. A61K 35/17; C12N 15/85; A61K 38/14; A61K 31/704; A61K 31/136

(52) U.S. Cl. CPC A61K 35/17; C12N 15/85; C12Q 2600/158; A61K 31/704; A61K 31/136; A61K 38/14

(57) ABSTRACT

This disclosure provides methods of treating cancer or eliciting an anti-tumor response in a subject by administering an effective amount of a population of T-cells that exhibits higher or lower than baseline expression of one or more genes. In other aspects, methods are provided to diagnose cancer and determine prognosis of cancer patients. Also provided are methods to identify the antigens or antigen receptors associated with the isolated and/or purified cell populations that elicit a more positive prognosis.

### CROSS-REFERENCE TO RELATED APPLICATIONS

This application claims priority under 35 U.S.C. § 119(e) to U.S. Provisional Application No. 62/647,588, filed Mar. 23, 2018, and U.S. Provisional Application No. 62/770,412, filed Nov. 21, 2018, the content of each which is hereby incorporated by reference in its entirety.

### BACKGROUND

High numbers of tissue-resident memory T (TRM) cells are associated with better clinical outcomes in cancer patients. However, the molecular characteristics that drive their efficient immune response to tumors are poorly understood. Thus, a need exists in the art to identify, characterize and harness these potent cells for therapeutic interventions. This disclosure satisfies this need and provides related advantages as well.

### SUMMARY OF THE DISCLOSURE

To address the above identified limitations in the art, this disclosure provides methods of treating cancer or eliciting an anti-tumor response in a subject in need thereof, the methods comprising, or consisting essentially of, or consisting of administering to the subject an effective amount of a population of T-cells that exhibits higher or lower than baseline expression of one or more select genes. In one aspect, this method comprises, or consists essentially of, or yet further consists of administering to the subject an effective amount of an active agent that induces higher or lower than baseline expression of one or more genes, or the one or more genes itself.

For the disclosed methods, in one aspect, the one or more genes are set forth in Table 1, Table 2, Table 3, Table 4, Table 5, or Table 7. In another aspect, the one or more genes are set forth in Table 1 and/or Table 2.

In other aspects, provided are one or more methods of diagnosing cancer, identifying a subject likely to benefit from or respond to cancer treatment, (including but not limited to immunotherapy (including anti-cancer or anti-tumor immunotherapy)), determining the effectiveness of cancer treatment, and/or determining a prognosis of a subject having cancer. The one or more methods comprise, or alternatively consist essentially of, or yet further consist of, detecting or measuring the population or amount of TRMs, or a sub-population of TRMs expressing high levels of one or more of, or all three TIM3, CXCL13 and CD39, in the subject or in a sample isolated from the subject. In certain embodiments, a higher amount of TRMs or higher amount of the sub-population of TRMs expressing high levels of TIM3, CXCL13 and CD39 in the subject or sample indicates that the subject is likely to benefit from or respond to cancer treatment, including immunotherapy (e.g., anti-cancer or anti-tumor immunotherapy), that the cancer treatment is effective in the subject, or that the subject is likely to proceed have a positive clinical response, e.g., longer overall survival, remission or longer time to tumor progression or lack of cancer recurrence. In certain embodiments, a lower amount of TRMs or lower amount of the sub-population of TRMs expressing high levels of one or more of or all three TIM3, CXCL13 and CD39 in the subject or sample indicates that the subject is less likely to benefit from or respond to cancer treatment, including immunotherapy (including anti-cancer or anti-tumor immunotherapy), that the cancer treatment is not as effective in the subject as other therapies, or that the subject has a poor prognosis with available therapies.

In certain aspects, the cells are T-cells, CD8+ T-cells, tumor-infiltrating lymphocytes (TILs), tissue-resident memory (Trm) cells. In certain other aspects, the T-cells and/or TRMs are CD19⁻CD20⁻CD14⁻CD56⁻CD4⁻CD45⁺CD3⁺CD8 cells. In certain aspects, the TRMs are TRMs expressing high levels of one or more of or all three of TIM3, CXCL13 and CD39.

This disclosure also provides the isolated or purified T-cell populations that are modified to exhibit higher or lower than baseline expression of one or more genes. In certain aspects, the T-cells are isolated and/or purified from a patient population using the markers provided herein, e.g., CD19⁻CD20⁻CD14⁻CD56⁻CD4⁻CD45⁺CD3⁺CD8 or modified expression of one or more of, or all three of TIM3, CXCL13 and CD39. In certain aspects, the isolated or purified T-cells including modified populations of same, are expanded to create homogeneous or heterogenous cell populations and/or combined with carriers, such as pharmaceutically acceptable carriers. In some aspect, the cell populations are administered to a subject in need thereof as an adoptive cell therapy. In certain aspects, T-cells are cells engineered or modified to reduce or eliminate expression and/or the function of one or more genes.

Also provided herein are methods to identify the antigens or antigen receptors associated with the isolated and/or purified cell populations disclosed herein. In some aspect, the receptors are T-cell receptors (TCRs). In particular embodiments, the TCRs comprise one or more of the sequences listed in Table 6. In certain embodiments, the identified antigens or antigen receptors are used to vaccinate or treat a subject against cancer, cancer progression or an immune response. In other aspects, the identified antigens or antigen receptors are used to engineer cells, for example a chimeric-antigen receptor T-cell (CAR-T cell). In still other aspects, the engineered CAR-T cell are used to provide immunotherapy to a subject in need thereof, such as for example, a human patient.

Also provided herein are methods to induce an immune response and treat conditions requiring selective immunotherapy, comprising, or consisting essentially of, or yet further consisting of, contacting a target cell with the cells or compositions as described herein. The contacting can be performed in vitro, or alternatively in vivo, thereby providing immunotherapy to a subject such as for example, a human patient.

In one aspect, the cancer or tumor is in head, neck, lung, lung, prostate, colon, pancreas, esophagus, liver, skin, kidney, adrenal gland, brain, or comprises a lymphoma, breast, endometrium, uterus, ovary, testes, lung, prostate, colon, pancreas, esophagus, liver, skin, kidney, adrenal gland, or brain. In other aspects, the cancer comprises a metastasis or recurring tumor, cancer or neoplasia. In certain aspects, the cancer comprises a non-small cell lung cancer (NSCLC) or head and neck squamous cell cancer (HNSCC).

Provided herein is a method of treating cancer and/or eliciting an anti-tumor response in a subject comprising, or consisting essentially of, or yet further consisting of administering to the subject an effective amount of a population of T-cells that exhibit higher than or lower than baseline expression of one or more genes set forth in Table 1, Table 2, Table 3, Table 4, Table 5 and/or Table 7, or that express a T-cell receptor comprising at least one of the amino acid sequences set forth in Table 6. In one aspect, the method comprises, or consists essentially of, or yet further consists of administering to the subject an effective amount of an agent that induces higher than or lower than baseline expression of one or more genes set forth in Table 1, Table 2, Table 3, Table 4, Table 5 and/or Table 7 in T-cells, or a T-cell receptor comprising at least one of the amino acid sequences set forth in Table 6. In another aspect, the method comprises, or consists essentially of, or yet further consists of administering an effective amount of one or more an agent that induces or inhibits in T-cells activity of one or more proteins encoded by genes set forth in Table 1, Table 2, Table 3, Table 4, Table 5 and/or Table 7 to the subject or sample. The active agent can be an antibody, a small molecule, a protein, a peptide, a ligand mimetic or a nucleic acid. The one or more gene may be selected from the group of 4-1BB, PD-1, CD103 or TIM3. In one aspect, the baseline expression is normalized mean gene expression. In another aspect, the higher than baseline expression is at least about a 2-fold increase in expression relative to baseline expression and/or lower than baseline expression is at least about a 2-fold decrease in expression relative to baseline expression. In a further aspect, the T-cells are tissue-resident memory cells (T_{RM}) or CD8+ T-cells. In one particular embodiment, the T-cells are autologous to the subject being treated. The methods of treating cancer and/or eliciting an anti-tumor response disclosed herein may further comprise, or consist essentially of, or yet further consist of administering to the subject an effective amount of a cytoreductive therapy. The cytoreductive therapy can be one or more of chemotherapy, immunotherapy, or radiation therapy.

Also disclosed herein is a modified T-cell modified to exhibit higher than or lower than baseline expression of one or more genes set forth in Table 1, Table 2, Table 3, Table 4, Table 5 and/or Table 7, or to express a T-cell receptor comprising, or consisting essentially of, or yet further consisting of at least one of the amino acid sequences set forth in Table 6. The one or more gene may be selected from the group of 4-1BB, PD-1, CD103 or TIM3. In one aspect, the baseline expression is normalized mean gene expression. In another aspect, the higher than baseline expression is at least about a 2-fold increase in expression relative to baseline expression and/or lower than baseline expression is at least about a 2-fold decrease in expression relative to baseline expression. In a further aspect, the T-cells are tissue-resident memory cells (T_{RM}) or CD8+ T-cells. In one particular embodiment, the T-cells are autologous to the subject being treated.

The modified T-cell can be genetically modified, optionally using recombinant methods and/or a gene editing technology such as TALENs or a CRISPR/Cas system. The modified T-cell disclosed herein can also be further modified to express a protein that binds to a cytokine, chemokine, lymphokine, or a receptor each thereof. In one aspect, the protein comprises, or consists essentially of, or yet further consists of an antibody or an antigen binding fragment thereof. In another aspect, the antibody is an IgG, IgA, IgM, IgE or IgD, or a subclass thereof. The antibody can also be an IgG selected from the group of IgG₁, IgG₂, IgG₃ or IgG₄. Furthermore, the antigen binding fragment can be selected from the group of a Fab, Fab′, F(ab′)2, Fv, Fd, single-chain Fvs (scFv), disulfide-linked Fvs (sdFv) or V_{L} or V_{H}.

In one aspect, the modified T-cell of this disclosure comprises, or consists essentially of, or yet further consists of modification that includes a chimeric antigen receptor (CAR). In one embodiment, the chimeric antigen receptor (CAR) comprises, or consists essentially of, or yet further consists of: (a) an antigen binding domain; (b) a hinge domain; (c) a transmembrane domain; (d) and an intracellular domain. The CAR can further comprise, or consist essentially of, or yet further consist of one or more costimulatory signaling regions. Further modifications are contemplated and within the scope of this disclosure, e.g., as reviewed in Ajina and Maher, (2018) Mol. Cancer Ther. 17(9):1795-1815. In one embodiment, the antigen binding domain comprises, or consists essentially of, or yet further consists of an anti-CD19 antigen binding domain, the transmembrane domain comprises, or consists essentially of, or yet further consists of a AMICA1, a CD28H (TMIGD2), a CD28 or a CD8α transmembrane domain and the one or more costimulatory regions selected from a CD28 costimulatory signaling region, a 4-1BB costimulatory signaling region, an AMICA1 costimulatory signaling region, a CD28H (TMIGD2) costimulatory signaling region, an ICOS costimulatory signaling region, and an OX40 costimulatory region or a CD3 zeta signaling domain. In a further embodiment, the anti-CD19 binding domain comprises, or consists essentially of, or yet further consists of a single-chain variable fragment (scFv) that specifically recognizes a humanized anti-CD19 binding domain. The anti-CD19 binding domain scFv of the CAR may comprise, or consist essentially of, or yet further consist of a heavy chain variable region and a light chain variable region.

In one aspect, the anti-CD19 binding domain of the CAR further comprises, or consists essentially of, or yet further consists of a linker polypeptide located between the anti-CD19 binding domain scFv heavy chain variable region and the anti-CD19 binding domain scFv light chain variable region. The linker polypeptide of the CAR may comprise, or consist essentially of, or yet further consist of a polypeptide of the sequence (GGGGS)n wherein n is an integer from 1 to 6. In another aspect, the CAR can further comprise, or consist essentially of, or yet further consist of a detectable marker attached to the CAR. In a separate aspect, the CAR can further comprise, or consist essentially of, or yet further consist of a purification marker attached to the CAR.

Further provided herein is a modified T-cell comprising, or consisting essentially of, or yet further consisting of a polynucleotide encoding the CAR, and optionally, wherein the polynucleotide encodes and anti-CD19 binding domain. In one aspect, the polynucleotide may further comprise, or consist essentially of, or yet further consist of a promoter operatively linked to the polynucleotide to express the polynucleotide in the modified T-cell. In another aspect, the polynucleotide may further comprise, or consist essentially of, or yet further consist of a 2A self-cleaving peptide (T2A) encoding polynucleotide sequence located upstream of a polynucleotide encoding the anti-CD19 binding domain. In yet a further aspect, the polynucleotide may further comprise, or consist essentially of, or yet further consist of a polynucleotide encoding a signal peptide located upstream of a polynucleotide encoding the anti-CD19 binding domain. In one embodiment, the polynucleotide further comprises, or consists essentially of, or yet further consists of a vector. In one particular embodiment, the vector is a plasmid. In another embodiment, the vector is a viral vector selected from the group of a retroviral vector, a lentiviral vector, an adenoviral vector, and an adeno-associated viral vector.

Also disclosed herein is a composition comprising, or consisting essentially of, or yet further consisting of a population of modified T-cells described above. Further provided herein is a method of treating cancer in a subject and/or eliciting an anti-tumor response comprising, or consisting essentially of, or yet further consisting of administering to the subject or contacting the tumor with an effective amount of the modified T-cells disclosed herein and/or the composition of this disclosure.

Further provided herein is a method of diagnosing a subject for cancer, comprising, or consisting essentially of, or yet further consisting of contacting a sample isolated from the subject with an agent that detects the presence of one or more genes set forth in Table 1, Table 2, Table 3, Table 4, Table Sand/or Table 7, wherein the presence of the one or more genes at higher or lower than baseline expression levels is diagnostic of cancer. In one aspect, the method comprises, or consists essentially of, or yet further consists of contacting tissue-resident memory cells (TRMs) isolated from the subject with an antibody or agent that recognizes and binds CD8, an antibody or agent that recognizes and binds PD-1, an antibody or agent that recognizes and binds TIM3, an antibody or agent that recognizes and binds LAG3, an antibody or agent that recognizes and binds AMICA1, an antibody or agent that recognizes and binds CD28H (TMIGD2), and an antibody or agent that recognizes and binds CTLA4 to determine the frequency of CD8⁺PD1⁺, CD8⁺TIM3⁺, CD8⁺LAG3⁺, CD8⁺AMICA1⁺, CD8⁺CD28H⁺, CD8⁺CTLA4⁺, CD8⁺PD1⁺TIM3⁺, CD8⁺PD1⁺LAG3⁺, CD8⁺PD1⁺AMICA1⁺, CD8⁺PD1⁺CD281-r CD8⁺PD1⁺CTLA4⁺′CD8⁺TIM3⁺LAG3⁺, CD8⁺TIM3⁺AMICA1⁺, CD8⁺TIM3⁺CD28H⁺, CD8⁺TIM3⁺CTLA4⁺, CD8⁺LAG3⁺CTLA4⁺, CD8⁺LAG3⁺AMICA1⁺, CD8⁺LAG3⁺CD28H⁺, CD8⁺PD1⁺TIM3⁺LAG3⁺, CD8+LAG3⁺PD1⁺AMICA1⁺, CD8⁺LAG3⁺PD1⁺CD28H⁺, CD8⁺PD1⁺LAG3⁺CTLA4⁺, CD8⁺PD1⁺TIM3⁺CTLA4⁺, CD8⁺PD1⁺TIM3⁺CTLA4⁺ AMICA1⁺′, CD8⁺PD1⁺TIM3⁺CTLA4⁺CD28H⁺, or CD8⁺PD1⁺TIM3⁺CTLA4⁺AMICA⁺CD28H⁺′ TRMs, wherein a high frequency of one or more of these TRMs is diagnostic of cancer.

In another aspect, the method of diagnosing cancer in a subject comprises, or consists essentially of, or yet further consists of contacting tissue-resident memory cells (TRMs) isolated from the subject with an antibody or agent that recognizes and binds one or more proteins encoded by a gene set forth in Table 1, Table 2, Table 3, Table 4, Table 5 and/or Table 7 and, optionally, an antibody or agent that recognizes and binds CD8, an antibody or agent that recognizes and binds PD-1, an antibody or agent that recognizes and binds TIM3, an antibody or agent that recognizes and binds LAGS, an antibody or agent that recognizes and binds S1PR1, an antibody or agent that recognizes and binds CD28H (TMIGD2), an antibody or agent that recognizes and binds AMICA1, an antibody or agent that recognizes and binds KLF3, an antibody or agent that recognizes and binds S1PR5, an antibody or agent that recognizes and binds KLF2 and an antibody or agent that recognizes and binds CTLA4 to determine the frequency of TRMs expressing these proteins, wherein a high frequency of TRMs expressing these proteins is diagnostic of cancer.

Additionally, disclosed herein is a method of determining the density of tissue-resident memory cells (TRMs) in a cancer, tumor, or sample isolated from the subject likely to contain these cells, the method comprising, or consisting essentially of, or yet further consisting of measuring expression of one or more gene selected from the group of 4-1BB, PD-1, CD103, AMICA1, CD28H or TIM3 or genes set forth in Table 1, Table 2, Table 3, Table 4, Table 5 and/or Table 7 in the cancer, tumor, or sample thereof, wherein higher or lower than baseline expression indicates higher density of TRMs in the cancer, tumor, or sample thereof.

Further provided herein is a method of determining prognosis of a subject having cancer comprising, or consisting essentially of, or yet further consisting of measuring the density of tissue-resident memory cells (T_{RM}) in the cancer or a sample isolated from the patient, wherein a high density of T_{RM} indicates a more positive prognosis, e.g., an increased probability and/or duration of survival. In one aspect, the method of prognosis of a subject having cancer comprises, or consists essentially of, or yet further consists of contacting tissue-resident memory cells (TRMs) isolated from the subject (e.g., of the cancer or a sample thereof) with an antibody or agent that recognizes and binds CD8, an antibody or agent that recognizes and binds PD-1, an antibody or agent that recognizes and binds TIM3, an antibody or agent that recognizes and binds LAG3, an antibody or agent that recognizes and binds AMICA1, an antibody or agent that recognizes and binds CD28H (TMIGD2), and an antibody or agent that recognizes and binds CTLA4 to determine the frequency of CD8⁺PD1⁺, CD8⁺TIM3⁺, CD8⁺LAG3⁺, CD8⁺AMICA1⁺, CD8⁺CD28H⁺, CD8⁺CTLA4⁺, CD8⁺PD1⁺TIM3⁺, CD8⁺PD1⁺LAG3⁺, CD8⁺PD1⁺AMICA1⁺, CD8⁺PD1⁺CD28H⁺, CD8⁺PD1⁺CTLA4⁺′CD8⁺TIM3⁺LAG3⁺, CD8⁺TIM3⁺AMICA1⁺, CD8⁺TIM3⁺CD28H⁺, CD8⁺TIM3⁺CTLA4⁺, CD8⁺LAG3⁺CTLA4⁺, CD8⁺LAG3⁺AMICA1⁺, CD8⁺LAG3⁺CD28H⁺, CD8⁺PD1⁺TIM3⁺LAG3⁺, CD8⁺LAG3⁺PD1⁺AMICA1⁺, CD8⁺LAG3⁺PD1⁺CD28H⁺, CD8⁺PD1⁺LAG3⁺CTLA4⁺, CD8⁺PD1⁺TIM3⁺CTLA4⁺, CD8⁺PD1⁺TIM3⁺CTLA4⁺AMICA1⁺′, CD8⁺PD1⁺TIM3⁺CTLA4⁺CD28H⁺′ or CD8⁺PD1⁺TIM3⁺CTLA4⁺AMICA⁺CD28H⁺′ TRMs, wherein a high frequency of one or more of these TRMs indicates a more positive prognosis, e.g., an increased probability and/or duration of survival. In another aspect, the method of prognosis of a subject having cancer comprises, or consists essentially of, or yet further consists of contacting tissue-resident memory cells (TRMs) of the cancer or a sample thereof with an antibody or agent that recognizes and binds one or more proteins encoded by a gene set forth in Table 1, Table 2, Table 3, Table 4, Table 5 and/or Table 7 and, optionally, an antibody or agent that recognizes and binds CD8, an antibody or agent that recognizes and binds PD-1, an antibody or agent that recognizes and binds TIM3, an antibody or agent that recognizes and binds LAG3, an antibody or agent that recognizes and binds S1PR1, an antibody or agent that recognizes and binds CD28H (TMIGD2), an antibody or agent that recognizes and binds AMICA1, an antibody or agent that recognizes and binds KLF3, an antibody or agent that recognizes and binds S1PR5, an antibody or agent that recognizes and binds KLF2 and an antibody or agent that recognizes and binds CTLA4 to determine the frequency of TRMs expressing these proteins, wherein a high frequency of TRMs expressing these proteins indicates a more positive prognosis, e.g., an increased probability and/or duration of survival.

In yet a further aspect, the method of determining prognosis of a subject having cancer comprises, or consists essentially of, or yet further consists of contacting tissue-resident memory cells (TRMs) isolated from the subject, (e.g., of the cancer or a sample thereof) with an antibody or agent that recognizes and binds CD103 to determine the frequency of CD103+ TRMs or an antibody or agent that recognizes and binds a protein encoded by a gene set forth in Table 1, Table 2, Table 3, Table 4, Table 5 and/or Table 7 to determine the frequency of TRMs expressing the protein, wherein a high or low frequency of TRMs expressing the protein indicates a more positive prognosis, e.g., an increased probability and/or duration of survival. In a separate aspect, the method of determining prognosis of a subject having cancer comprises, or consists essentially of, or yet further consists of measuring the density of CD103 or proteins encoded by one or more gene set forth in Table 1, Table 2, Table 3, Table 4, Table 5 and/or Table 7 in the sample (e.g., cancer or a sample thereof), wherein a high or low density of proteins indicates a more positive prognosis, e.g., an increased probability and/or duration of survival.

Also described herein is a method of determining the responsiveness of a subject having cancer to immunotherapy comprising, or consisting essentially of, or yet further consisting of contacting tissue-resident memory cells (TRMs) isolated from the subject, (e.g., of the cancer or a sample thereof) with an antibody or agent that recognizes and binds CD8, an antibody or agent that recognizes and binds PD-1, an antibody or agent that recognizes and binds TIM3, an antibody or agent that recognizes and binds AMICA1, an antibody or agent that recognizes and binds CD28H (TMIGD2), an antibody or agent that recognizes and binds LAG3, and an antibody or agent that recognizes and binds CTLA4 to determine the frequency of CD8⁺PD1⁺, CD8⁺TIM3⁺, CD8⁺LAG3⁺, CD8⁺AMICA1⁺, CD8⁺CD28H⁺, CD8⁺CTLA4⁺, CD8⁺PD1⁺TIM3⁺, CD8⁺PD1⁺LAG3⁺, CD8⁺PD1⁺AMICA1⁺, CD8⁺PD1⁺CD28H⁺, CD8⁺PD1⁺CTLA4⁺, CD8⁺TIM3⁺LAG3⁺, CD8⁺TIM3⁺AMICA1⁺, CD8⁺TIM3⁺CD28H⁺, CD8⁺TIM3⁺CTLA4⁺, CD8⁺LAG3⁺CTLA4⁺, CD8⁺LAG3⁺AMICA1⁺, CD8⁺LAG3⁺CD28H⁺, CD8⁺PD1⁺TIM3⁺LAG3⁺, CD8⁺LAG3⁺PD1⁺AMICA1⁺, CD8⁺LAG3⁺PD1⁺CD28H⁺, CD8⁺PD1⁺LAG3⁺CTLA4⁺, CD8⁺PD1⁺TIM3⁺CTLA4⁺, CD8⁺PD1⁺TIM3⁺CTLA4⁺AMICA1⁺′, CD8⁺PD1⁺TIM3⁺CTLA4⁺CD28H⁺′ or CD8⁺PD1⁺TIM3⁺CTLA4⁺AMICA⁺CD28H⁺′ TRMs, wherein a high frequency of one or more of these TRMs indicates responsiveness to immunotherapy. In one aspect, the method of determining the responsiveness of a subject having cancer to immunotherapy comprises, or consists essentially of, or yet further consists of contacting tissue-resident memory cells (TRMs) isolated from the subject, (e.g., of the cancer or a sample thereof) with an antibody that recognizes and binds one or more proteins encoded by a gene set forth in Table 1, Table 2, Table 3, Table 4, Table 5 and/or Table 7 and, optionally, an antibody or agent that recognizes and binds CD8, an antibody or agent that recognizes and binds PD-1, an antibody or agent that recognizes and binds TIM3, an antibody or agent that recognizes and binds LAG3, an antibody or agent that recognizes and binds CD28H (TMIGD2), an

antibody or agent that recognizes and binds AMICA1, an antibody or agent that recognizes and binds KLF3, an antibody or agent that recognizes and binds S1PR5, an antibody or agent that recognizes and binds S1PR1, an antibody or agent that recognizes and binds KLF2 and an antibody or agent that recognizes and binds CTLA4 to determine the frequency of TRMs expressing these proteins, wherein a high frequency of TRMs expressing these proteins indicates responsiveness to immunotherapy. For any of the methods disclosed herein, the TRMs may comprise, or consist essentially of, or yet further consist of CD19⁻CD20⁻CD14⁻CD56⁻CD4⁻CD45⁺CD3⁺CD8⁺ T-cells.

Further disclosed are methods of identifying a subject that will or is likely to respond to a cancer therapy, comprising, or consisting essentially of, or yet further consisting of contacting the same with an agent that detects the presence of one or more genes set forth in Table 1, Table 2, Table 3, Table 4, Table 5 and/or Table 7 in a sample isolated from the subject, (e.g., the cancer or a sample thereof), wherein the presence of the one or more genes at higher or lower than baseline expression levels indicates that the subject is likely to respond to cancer therapy. In one aspect, the baseline expression is normalized mean gene expression. In another aspect, the higher than baseline expression is at least about a 2-fold increase in expression relative to baseline expression and/or lower than baseline expression is at least about a 2-fold decrease in expression relative to baseline expression. The method may further comprise, or consist essentially of, or yet further consist of administering a cancer therapy to the subject. The cancer therapy or cytoreductive therapy can be chemotherapy, immunotherapy, radiation therapy, and/or administering to the subject or contacting the tumor with an effective amount of the modified T-cells and/or the composition of this disclosure.

The cancer, tumor, or sample can be contacted with an agent, optionally including a detectable label or tag. In one aspect, the detectable label or tag can comprise, or consist essentially of, or yet further consist of a radioisotope, a metal, horseradish peroxidase, alkaline phosphatase, avidin or biotin. In another aspect, the agent can comprise, or consist essentially of, or yet further consist of a polypeptide that binds to an expression product encoded by the gene, or a polynucleotide that hybridizes to a nucleic acid sequence encoding all or a portion of the gene. The polypeptide may comprise, or consist essentially of, or yet further consist of an antibody, an antigen binding fragment thereof, or a receptor that binds to the gene. In one aspect, the antibody is an IgG, IgA, IgM, IgE or IgD, or a subclass thereof. In another aspect, the IgG antibody is an IgG₁, IgG₂, IgG₃ or IgG₄. The antigen binding fragment can be a Fab, Fab′, F(ab′)2, Fv, Fd, single-chain Fvs (scFv), disulfide-linked Fvs (sdFv) or V_{L} or V_{H}. In one aspect, the agent is contacted with the cancer, tumor, or sample in conditions under which it can bind to the gene it targets.

The methods of this disclosure comprise, or consist essentially of, or yet further consist of detection by immunohistochemistry (IHC), in-situ hybridization (ISH), ELISA, immunoprecipitation, immunofluorescence, chemiluminescence, radioactivity, X-ray, nucleic acid hybridization, protein-protein interaction, immunoprecipitation, flow cytometry, Western blotting, polymerase chain reaction, DNA transcription, Northern blotting and/or Southern blotting. The sample may comprise, or consist essentially of, or yet further consist of cells, tissue, an organ biopsy, an epithelial tissue, a lung, respiratory or airway tissue or organ, a circulatory tissue or organ, a skin tissue, bone tissue, muscle tissue, head, neck, brain, skin, bone and/or blood sample. While the cancer or tumor described herein can be an epithelial, a head, neck, lung, lung, prostate, colon, pancreas, esophagus, liver, skin, kidney, adrenal gland, brain, or comprises a lymphoma, breast, endometrium, uterus, ovary, testes, lung, prostate, colon, pancreas, esophagus, liver, skin, kidney, adrenal gland and/or brain cancer or tumor, a metastasis or recurring tumor, cancer or neoplasia, a non-small cell lung cancer (NSCLC) and/or head and neck squamous cell cancer (HNSCC).

In a further aspect, the methods of this disclosure comprise, or consist essentially of, or yet further consist of, detecting in the subject, in the cells or in a sample isolated from the subject, the number or density of Trm cells that are CD19-CD20-CD14-CD56-CD4-CD45⁺CD3⁺CD8+ T-cells.

Finally, provided herein is a kit comprising, or consisting essentially of, or yet further consisting of one or more of the modified T-cells and/or the composition of this disclosure and instructions for use. In one aspect, the instruction for use provide directions to conduct any of the methods described herein.

### BRIEF DESCRIPTION OF THE DRAWINGS

The drawings illustrate embodiments of the technology and are not limiting. For clarity and ease of illustration, the drawings are not made to scale, and, in some instances, various aspects may be shown exaggerated or enlarged to facilitate an understanding of particular embodiments.

FIGS. 1A-1F: CD103 expressing CTLs in human lungs are enriched for tissue residency features but are transcriptionally distinct from previously characterized TRM cells. (FIG. 1A) tSNE plot of lung TRM (CD103⁺) and non-T_{RM} (CD103⁻) CTLs. Each symbol represents an individual patient sample (n=21 non-T_{RM}; n=20 T_{RM}). (FIG. 1B) RNA-Seq analysis of transcripts (one per row) expressed differentially between lung T_{RM} and lung non-T_{RM}, (pairwise comparison; change in expression of 2-fold with an adjusted P value of <0.05 (DESeq2 analysis; Benjamini-Hochberg test)), presented as row-wise z-scores of transcripts per million (TPM). Each column represents an individual sample; key known TRM or non-T_{RM} transcripts are indicated. Color scheme and number of samples is identical to (FIG. 1A). (FIG. 1C) GSEA of the murine composite TRM signature in the transcriptome of lung TRM vs. lung non-T_{RM}: top, running enrichment score (RES) for the gene set, from most over-represented genes at left to most under-represented at right; middle, positions of gene set members (blue vertical lines) in the ranked list of genes; bottom, value of the ranking metric. Values above the plot represent the normalized enrichment score (NES) and false discovery rate (FDR)-corrected significance value. (FIG. 1D) Flow-cytometry analysis of the expression of CD49A and KLRG1 versus that of CD103 among live and singlet-gated CD19⁻CD20⁻CD14⁻CD45⁺CD3⁺CD8⁺ cells obtained from lung; right, frequency of CD103+ CTLs or CD103⁻ CTLs that express the indicated surface marker (*P≤0.05, n=6), bars represent the mean, t-line the s.e.m., and symbol represents data from individual samples. (FIGS. 1E-1F) Venn diagrams (upper) showing overlap of transcripts differentially expressed in lung TRM versus other previously characterized TRM cells. Waterfall plots (lower) represent the DESeq2 normalized fold change of genes not significantly (<2-fold) differentially expressed between lung TRM (CD103⁺) and non-T_{RM} (CD103⁻) CTLs.

FIGS. 2A-2H: TRM cells in normal lung and lung tumors share tissue residency features but are otherwise distinct. (FIG. 2A) GSEA of murine composite TRM signature in the transcriptome of lung tumor TRM vs. that of tumor non-T_{RM} cells; top, running enrichment score (RES) for the gene set, from most over-represented genes at left to most under-represented at right; middle, position of gene set members (blue vertical lines) in the ranked list of genes; bottom, value of the ranking metric. Values above the plots represent the normalized enrichment score (NES) and FDR-corrected significance value. (FIG. 2B) tSNE plot of tumor and lung CTL transcriptomes segregated by CD103 expression (lung non-T_{RM}=21, lung TRM=20, tumor non-T_{RM}=25, tumor TRM=19). (FIG. 2C) Venn diagram and (FIG. 2D) heat map of RNA-Seq analysis of 89 common transcripts (one per row) expressed differentially by lung TRM versus lung non-T_{RM}, and tumor TRM versus tumor non-T_{RM} (pairwise comparison; change in expression of 2-fold with an adjusted P value of <0.05 (DESeq2 analysis; Benjamini-Hochberg test)), presented as row-wise z-scores of TTPM; each column represents an individual sample; key known TRM or non-T_{RM} transcripts are indicated. Color scheme and number of samples is identical to (FIG. 2B). (FIG. 2E) Spearman co-expression analysis of the 89 differentially expressed genes as in (c) and (d); values are clustered with complete linkage. A topological overlap matrix was calculated at power 5 using weighted gene co-expression network analysis and visualized in Gephi. The nodes are colored and sized according to the number of edges (connections), and the edge thickness is proportional to the edge weight (strength of correlation). The network layout is assigned by the Fruchterman-Reingold algorithm, using Noverlap to prevent overlapping labels. (FIG. 2F) Quantitated expression according to RNA-Seq data of the indicated differentially expressed genes shared by lung and tumor TRM cells. Each symbol represents an individual sample, the bar represents the mean and colored as in (FIG. 2B), t-line the s.e.m. (FIG. 2G) Flow-cytometry analysis of the expression of PD1 versus that of CD103 on live and singlet-gated CD19⁻CD20⁻CD14⁻CD56⁻CD4⁻CD45⁺CD3⁺CD8⁺ cells obtained from lung cancer TILs; right, frequency of cell that express PD-1 in the indicated populations (* P≤0.05; n=8), each symbol represents a sample, bars represent the mean, t-line the s.e.m. (FIG. 2H) RNA-Seq analysis of genes (row) up- or downregulated in the 4 cell types following 4 h of ex vivo stimulation. Left, heat map as in (FIG. 2D); right, bar graphs showing expression of transcripts in the indicated populations (n=6 for all comparisons; represented as in (FIG. 2F)).

FIGS. 3A-3F: Tumor T_{RM} cells proliferate, express the inhibitory checkpoint TIM3 and markers of enhanced function. (FIG. 3A) RNA-Seq analysis of transcripts (one per row) differentially expressed by tumor TRM relative to lung T_{RM}, lung non-T_{RM}, and tumor non-T_{RM} (pairwise comparison; change in expression of 2-fold with an adjusted P value of <0.05 (DESeq2 analysis; Benjamini-Hochberg test)), presented as row-wise z-scores of TPM; each column represents an individual sample (lung non-T_{RM}=21, lung TRM=20, tumor non-T_{RM}=25, tumor TRM=19). (FIG. 3B) Summary of over-representation analysis (using Reactome) of genes involved in the cell cycle that are differentially expressed by lung tumor T_{RM} relative to the other lung CTLs; q values represent false discovery rate (FIG. 3C) Shannon-Wiener diversity and Inverse Simpson indices obtained using V(D)J tools following TCR-seq analysis of β chains in tumor TRM and tumor non-T_{RM} populations. Bars represent the mean, t-line the s.e.m., and symbols represent individual data points (**P<0.01; n=10 patients). (FIG. 3D) Left, bar graphs show the percentage of total TCRβ chains that were expanded (≥3 clonotypes). Bars represent the mean, t-line the s.e.m., and dots individual data points (** P≤0.01; n=10 patients). Right, pie charts show the distribution of TCRβ clonotypes based on clonal frequency. (FIG. 3E) Left, Spearman co-expression analysis of the 77 genes up-regulated (FIG. 3A) in tumor TRM cells; values are clustered with complete linkage. Right, topological overlap matrix calculated at power 5 using weighted gene co-expression network analysis and visualized in Gephi. Node color and size are scaled according to the number of edges, edge thickness is proportional to the weight, and the network layout is assigned by the Fruchterman-Reingold algorithm, using Noverlap to prevent overlapping labels. (FIG. 3F) Correlation of the expression of HAVCR2 (TIM3) transcripts and the indicated transcripts in tumor TRM population; r indicates Spearman correlation value (*P≤0.05; *** P≤0.001; **** P≤0.0001).

FIGS. 4A-4G: Single-cell transcriptomic analysis reveals previously uncharacterized TRM subsets. (FIG. 4A) tSNE visualization of ˜12,000 live and singlet-gated, CD19⁻CD20⁻CD14⁻CD4⁻CD56⁻CD3⁺CD45⁺CD8⁺ single cell transcriptomes obtained from 12 tumors and 6 matched normal lung samples. Each symbol represents a cell; color indicates protein expression of CD103 detected by flow cytometry. (FIG. 4B) Seurat clustering of cells in (FIG. 4A) identifying 9 clusters. (FIG. 4C) Cells from tumor and lung were randomly downsampled to equivalent numbers of cells. Left, distribution of T_{RM}-enriched clusters in tumor and lung. Right, pie chart representing the relative proportions of cells in each TRM cluster. (FIG. 4D) Expression of transcripts previously identified as upregulated in the bulk tumor TRM population (FIG. 3A) by each cluster; each column represents the average expression in a particular cluster. (FIG. 4E) Breakdown of cell type and tissue localization of cells defined as being in cluster 1. (FIG. 4F) Violin plots of expression of example tumor TRM genes in each T_{RM}-enriched cluster (square below indicates the cluster type); shape represents the distribution of expression among cells and color represents the Seurat-normalized average expression. (FIG. 4G) Cell-state hierarchy maps generated by Monocle2 bioinformatics modeling of the TRM clusters; center plot, each dot represents a cell colored according to Seurat-assigned assigned cluster; surrounding panels show relative Seurat-normalized expression of the indicated genes.

FIGS. 5A-5D: A subset of tumor TRM cells has a transcriptional program indicative of superior functional properties. (FIG. 5A) Single-cell RNA-Seq analysis of transcripts (one per row) uniquely differentially expressed by each tumor TRM subset in pairwise analysis compared to other clusters (adjusted P value of <0.01; MAST analysis), presented as row-wise z-scores of Seurat-normalized count, each column represents an individual cell. Horizontal breaks separate genes enriched in each of the 4 tumor TRM subtypes. (FIG. 5B) Seurat-normalized expression of indicated transcripts identified as differentially enriched in each cluster, overlaid across the tSNE plot, with expression levels represented by the color scale. (FIG. 5C) Violin plot of expression of functionally important genes identified as significantly enriched in the ‘highly functional’ TRM subset; shape represents the distribution of expression among cells and color represents the Seurat-normalized average expression. The 91 transcripts enriched in cluster 2 compared to the other TRM clusters included several which encoded products linked to cytotoxic activity such as PRF1, GZMB, GZMA, CTSW³⁸, and CRTAM³⁸, as well as transcripts encoding effector cytokines and chemokines such as IFNγ, CCL3, CXCL13, IL17A and IL26. TRM cells exhibited a transcriptional program suggestive of superior effector properties and cell proliferation expressed high transcript levels for cytotoxicity molecules (Perforin, Granzyme A and Granzyme B) and several co-stimulatory molecules such as 4-1BB, ICOS and GITR (TNFRSF18). (FIG. 5D) Top, violin plot of expression of genes encoding key effector molecules in specific tumor-infiltrating CTL subsets. Below, percentage of cells expressing IFNG transcripts in each population, where positive expression was defined as greater than 1 Seurat-normalized count; “Other T_{RM}” corresponds to tumor CTLs isolated from clusters 3, 4, and 5.

FIGS. 6A-6J: PD-1- and TIM3-expressing tumor-infiltrating TRM lack an exhausted phenotype and exhibit enhanced clonal expansion. (FIG. 6A) GSEA of ‘highly functional’ TRM signature in the transcriptome of clonally expanded tumor TRM vs. that of non-expanded TRM cells: top, running enrichment score (RES) for the gene set, from most over-represented at left to most under-represented at right; middle, positions of gene set members (blue vertical lines) in the ranked list of genes; bottom, value of the ranking metric. Values above the plot represent the normalized enrichment score (NES) and FDR-corrected significance. (FIG. 6B) Left, percentage of cells that were clonally expanded in TIM3⁺ (HAVCR2>10 TPM) TRM cells, remaining TRMs and non-T_{RM}; clonal expansion was determined for cells from 4 and 2 patients for TRM and non-T_{RM}, respectively. Right, clonotype network graphs of cells from a representative donor. TIM3⁺ (HAVCR2>10 TPM) TRM cells are marked with a circle; cells with greater than 10 T_{RM} expression of either MKI67 or TOP2A were considered cycling and denoted with an 6 asterisk. (FIG. 6C) Violin plot of expression of indicated transcripts; Shape represents the distribution of expression among cells and color represents average expression, calculated from the TPM. (FIG. 6D) Correlation of PDCD1 and IFNG expression in TIM3+ TRM and non-T_{RM} cells; each dot represents a cell. Percentages indicate the percentage of cells inside each of the graph sections (r indicates Spearman correlation value; ** P≤0.01, ns=no significance). (FIG. 6E) Spearman co-expression analysis of genes whose expression is enriched in the ‘hyper functional’ TRM cluster (FIG. 5A) in tumor TRM and non-T_{RM} populations, respectively; matrix is clustered according to gene linkage. (FIG. 6F) tSNE visualization of flow cytometry data from 3,000 randomly selected live and singlet-gated CD19⁻CD20⁻CD14⁻CD56⁻CD4⁻CD45⁺CD3⁺CD8⁺ cells isolated from 8 paired tumor and lung samples; each cell is represented by a dot colored as TRM or non-T_{RM} (left), tumor or lung (second left), and according to Z-score expression value of the protein indicated above the plot (remaining panels). (FIG. 6G) Applicants' plots show expression of TIM3 versus IL7R in the cell type and tissue indicated above the plot; percentage of TIM3⁺ cells in the indicated populations is shown (right), each symbol represents an individual sample; the small line indicates the s.e.m, bars are mean and colored as indicated (*P≤0.05; n=8). (FIG. 6H) Right, geometric mean fluorescent intensity (GMFI) of CD39, PD1 and 41BB for each tumor TRM subset; bars represent the mean, t-line the s.e.m., and symbol represents data from individual samples (**P≤0.01; n=8); representative histograms shown (left). (FIG. 6I) Co-expression analysis of flow cytometry data (FIG. 6F), as per Spearman correlation value, matrix is clustered by complete linkage. (FIG. 6J) Spearman correlation of HACVR2 (TIM3) expression with ITGAE (CD103) expression in bulk transcriptomic profiles of CTLs isolated from lung cancer and head and neck squamous cell carcinoma.

FIG. 7: Cell sorting strategy. Plots describe the sorting strategy used for isolating immune cell types from tissue samples.

FIG. 8: Validation of lung TRM phenotype. Flow-cytometry analysis of the expression of KLRG1 and CD49A versus that of CD103 in live, singlet CD19⁻CD20⁻CD14⁻CD45⁺CD3⁺CD8⁺ cells obtained from lung samples (n=6).

FIG. 9: Validation of PD1 expression. Flow-cytometry analysis of the expression of PD-1 versus that of CD103 in live, singlet CD19⁻CD20⁻CD14⁻CD4⁻CD56⁻CD45⁺CD3⁺CD8⁺ cells obtained from lung and tumor samples (n=8).

FIGS. 10A-10D: T_{RM} cluster into 4 major subtypes. (FIG. 10A) Principle component analysis of the single cell transcriptomes, each point represents a cell which are colored as per the cluster assignment in FIG. 5; numbers along perimeter indicate principal components (PC1-PC3). (FIG. 10B) tSNE visualization of single cell transcriptomes, shown per donor (as per FIG. 4a), obtained from 12 tumors and 6 matched normal lung samples. Each symbol represents a cell; color indicates Seurat clustering of cells, as per FIG. 4b, identifying 9 clusters. (FIG. 10C) Breakdown of cells assigned to each cluster in each donor, separated by tissue type of origin (colored as per FIG. 4B). (FIG. 10D) The distance between a cell assigned to cluster 1 compared to the mean of cells assigned into the other clusters (colored as per b). The difference was calculated with the raw (left) and z-score normalized (right) distances, bars represent the mean distance to each of the other clusters, t-line the s.e.m., and symbols represent individual cells in cluster 1 (**** P≤0.0001; Wilcoxon matched-pairs signed rank test, n=135 cells).

FIGS. 11A-11B: ‘Highly-functional’ TRM cells are enriched for transcripts associated with enhanced anti-tumor features. (FIG. 11A) Violin plot of expression of indicated transcripts; shape represents the distribution of expression among cells and color represents average expression, calculated from the Seurat-normalized counts. (FIG. 11B) SAVER-imputed spearman co-expression analysis of genes whose expression is enriched in the TIM-3+IL7R− TRM cluster (FIG. 5A) in tumor TRM and non-TRM clusters, respectively; matrix is clustered according to complete linkage.

FIG. 12: TIM3-expressing TRM cells are enriched for co-expression of PD1 and cytotoxicity-related transcripts. Single-cell RNA-Seq analysis of transcripts (one per row) differentially expressed by TIM3⁺^{^{TRM}} relative to non-TIM3⁺ (MAST analysis with an adjusted P value of <0.05), presented as row-wise z-scores of TPM; each column represents a single cell (n=89 and 411, respectively).

FIGS. 13A-13C: Tumor TRMS are enriched for TIM3⁺ cells. (FIG. 13A) Flow-cytometry analysis of the expression of TIM3 versus that of CD103 in live, singlet CD19⁻CD20⁻CD14⁻CD4⁻CD56⁻CD45⁺CD3⁺CD8⁺ cells obtained from lung, lung tumor and HNSCC samples (n=8,8,3, respectively). (FIG. 13B) Flow-cytometry analysis of TIM3 compared to IL7R in CD103⁺ cells gated as in (FIG. 13A). (FIG. 13C) Quantification (geometric mean) of indicated marker (above) in HNSCC cells gated as in (FIG. 13B), bars represent the mean, t-line the s.e.m., and symbol represents data from individual samples (n=3).

FIG. 14: Analysis of AMICA1 expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Middle) Percent of cells expressing a given transcript in each cluster. (Lower) Expression values according to the log₂+1 transformed RNA-seq transcripts per million of the indicated differentially expressed genes shared by lung and tumor TRM cells. Each symbol represents an individual sample, the bar represents the mean and colored as described in the legend, t-line the s.e.m.

FIG. 15: Analysis of SPRY1 expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Middle) Percent of cells expressing a given transcript in each cluster. (Lower) Expression values according to the log₂+1 transformed RNA-seq transcripts per million of the indicated differentially expressed genes shared by lung and tumor TRM cells. Each symbol represents an individual sample, the bar represents the mean and colored as described in the legend, t-line the s.e.m.

FIG. 16: Analysis of CHN1 expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 17: Analysis of PAG1 expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 18: Analysis of PTPN22 expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 19: Analysis of DUSP4 expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 20: Analysis of ICOS expression. (Upper) tSNE visualization of ˜42,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 21: Analysis of TNFRSF18 (GITR) expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 22: Analysis of TMIGD2 (CD28H) expression. (Upper) tSNE visualization of ˜12,000 live and singlet-gated CD14⁻CD19⁻CD20⁻CD4⁻CD56⁻CD3±CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Middle) Percent of cells expressing a given transcript in each cluster. (Lower) Expression values according to the log₂+1 transformed RNA-seq transcripts per million of the indicated differentially expressed genes shared by lung and tumor TRM cells. Each symbol represents an individual sample, the bar represents the mean and colored as described in the legend, t-line the s.e.m.

FIG. 23: Analysis of CD226 expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Middle) Percent of cells expressing a given transcript in each cluster. (Lower) Expression values according to the log₂+1 transformed RNA-seq transcripts per million of the indicated differentially expressed genes shared by lung and tumor TRM cells. Each symbol represents an individual sample, the bar represents the mean and colored as described in the legend, t-line the s.e.m.

FIG. 24: Analysis of TIGIT expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 25: Analysis of KLRC1 (NKG2A) expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Middle) Percent of cells expressing a given transcript in each cluster. (Lower) Expression values according to the log₂+1 transformed RNA-seq transcripts per million of the indicated differentially expressed genes shared by lung and tumor TRM cells. Each symbol represents an individual sample, the bar represents the mean and colored as described in the legend, t-line the s.e.m.

FIG. 26: Analysis of KLRC2 (NKG2C) expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Middle) Percent of cells expressing a given transcript in each cluster. (Lower) Expression values according to the log₂+1 transformed RNA-seq transcripts per million of the indicated differentially expressed genes shared by lung and tumor T_{RM} cells. Each symbol represents an individual sample, the bar represents the mean and colored as described in the legend, t-line the s.e.m.

FIG. 27: Analysis of CAPG expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Middle) Percent of cells expressing a given transcript in each cluster. (Lower) Expression values according to the log₂+1 transformed RNA-seq transcripts per million of the indicated differentially expressed genes shared by lung and tumor TRM cells. Each symbol represents an individual sample, the bar represents the mean and colored as described in the legend, t-line the s.e.m.

FIG. 28: Analysis of MYO1E expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 29: Analysis of CLEC2B expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 30: Analysis of CLECL1 expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 31: Analysis of TNFRSF9 (4-1BB/CD137) expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 32: Analysis of TNFSF4 (CD134L/OX40L) expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 33: Analysis of NR3C1 (glucocorticoid receptor) expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 34: Analysis of CD7 expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 35: Analysis of KLRD1 (CD94) expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 36: Analysis of CLEC2D expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 37: Analysis of ITM2A expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 38: Analysis of VCAM1 (CD106) expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 39: Analysis of KRT81 expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 40: Analysis of KRT86 expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 41: Analysis of CXCL13 expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 42: Analysis of CBLB expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 43: Analysis of KLRC3 (NKG2-E) expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Middle) Percent of cells expressing a given transcript in each cluster. (Lower) Expression values according to the log₂+1 transformed RNA-seq transcripts per million of the indicated differentially expressed genes shared by lung and tumor T_{RM} cells. Each symbol represents an individual sample, the bar represents the mean and colored as described in the legend, t-line the s.e.m.

FIG. 44: Analysis of KLRB1 (CD161) expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Middle) Percent of cells expressing a given transcript in each cluster. (Lower) Expression values according to the log₂+1 transformed RNA-seq transcripts per million of the indicated differentially expressed genes shared by lung and tumor TRM cells. Each symbol represents an individual sample, the bar represents the mean and colored as described in the legend, t-line the s.e.m.

FIG. 45: Analysis of CD101 expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Middle) Percent of cells expressing a given transcript in each cluster. (Lower) Expression values according to the log₂+1 transformed RNA-seq transcripts per million of the indicated differentially expressed genes shared by lung and tumor TRM cells. Each symbol represents an individual sample, the bar represents the mean and colored as described in the legend, t-line the s.e.m.

FIG. 46: Analysis of CD101 expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Middle) Percent of cells expressing a given transcript in each cluster. (Lower) Expression values according to the log₂+1 transformed RNA-seq transcripts per million of the indicated differentially expressed genes shared by lung and tumor TRM cells. Each symbol represents an individual sample, the bar represents the mean and colored as described in the legend, t-line the s.e.m.

FIG. 47: Analysis of CD200R1 expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Middle) Percent of cells expressing a given transcript in each cluster. (Lower) Expression values according to the log₂+1 transformed RNA-seq transcripts per million of the indicated differentially expressed genes shared by lung and tumor TRM cells. Each symbol represents an individual sample, the bar represents the mean and colored as described in the legend, t-line the s.e.m.

FIG. 48: Analysis of SLA (SLAP) expression. (Upper) tSNE visualization of ˜12,000 live and singlet-gated CD14⁻CD19⁻CD20⁻CD4⁻CD56⁻CD3⁺CD45⁺CD8⁺ single cell transcriptomes obtained from 12 tumors and 6 matched normal lung samples, the TIM3⁺IL7R⁻ T_{RM} cluster is found in the bottom right. Expression is calculated using Seurat normalized counts. (Lower) Percent of cells expressing a given transcript in each cluster.

FIG. 49: CD103 density predicts survival in lung cancer. CD103 density (CD103high, CD103int, CD103low) in tumors pre-classified based on CD8 density (left); Kaplan-Meier curves for lung cancer mortality in CD8high tumors sub-classified according to density of CD103 (right).

FIGS. 50A-50B: (FIG. 50 A) Flow-cytometry analysis of the percentage of PD-1+T_{RM} and PD-1+ non-TRM cells that express effector cytokines following 4 hours of ex-vivo stimulation. Gated on live and singlet-gated CD14−CD20−CD4−CD45+CD3+CD8+ cells obtained from lung cancer TILs, discriminated on CD103 expression (**≤0.01; Wilcoxon matched pairs signed rank test; n=11), each symbol represents a sample. Surface molecules (e.g., PD-1) were stained before stimulation. (FIG. 50 B) Analysis of Granzyme A and Granzyme B directly ex-vivo, gated and analyzed as per a) (*** P≤0.001).

FIG. 51: Left, quantification of the number of CD8A+CD103+TIM-3+ cells per region in biopsies defined as having a TILhigh/TRM high status versus. TILlow/TRM low status. Right, percent of CD8A+CD103+ CTLs expressing TIM-3 in each clinical subtype. Bars represent the mean, t-line the s.e.m., and symbols represent individual data points (* P≤0.05; **** P≤0.0001; n=21).

FIGS. 52A-52K: (FIG. 52A) Representative FACS plots to characterize tumor-infiltrating CD19⁺, CD4⁺ and CD8⁺ T cells from mice at d21 after inoculation with B16F10-OVA cells. (FIG. 52B, FIG. 52C) MFI of AMICA1 expression of CD19⁺, CD4⁺and CD8⁺ TILs as in (FIG. 52A). (FIG. 52D) Frequency of AMICA1 expressing CD19⁺, CD4⁺ and CD8⁺ TILs as in (FIG. 52A). (FIG. 52E, FIG. 52F) Representative FACS plots (FIG. 52E) depicting cell viability, electroporation efficiency, antigen specificity and knockdown efficiency (FIG. 52F) of purified, in vitro activated and electroporated OT-I CD8⁺ T cells at 96 h after electroporation. Cells were electroporated to introduce gRNAs targeting a control region (ctrl) or AMICA1. (FIG. 52G, FIG. 52H, FIG. 52I) Representative FACS plots from mice at d20 after inoculation with B16F10-OVA cells. CD45.2 OT-I control and AMICA-1^{-/-} T cells were adoptively transferred at d6 after tumor inoculation. (FIG. 52J) Growth curves of B16F10-OVA tumors after adoptive transfer of OT-I and AMICA-1^{-/-} T cells. (FIG. 52K) Growth curves of B16F10-OVA tumors after treatment at d10 and d13 with 200 ug anti-PD-1, anti-AMICA-1 or anti isotype control antibodies.

FIG. 53: Left, quantification of the number of CD8A+CD103+TIM-3+ cells per region in biopsies defined as having a TILhigh/TRM high status versus. TILlow/TRM low status. Right, percent of CD8A+CD103+ CTLs expressing TIM-3 in each clinical subtype. Bars represent the mean, t-line the s.e.m., and symbols represent individual data points (* P≤0.05; **** P≤0.0001; n=21).

FIGS. 54A-54H: Single-cell transcriptome analysis. Left, contour plots show the expression of TIM-3 and IL-7R in CD14−CD19−CD20−CD4−CD45+CD3+CD8+CD103+ cells isolated from patients receiving anti-PD-1 treatment, at the time point indicated above the plot (TP); number in bottom right indicates the percentage of tumor T_{RM} cells (CD8+CD103+) with TIM-3+IL-7R− surface phenotype. Right, quantification of the percentage of tumor-infiltrating TIM-3+IL-7R− TRM cells, isolated from the anti-PD1 responding, non-responding and treatment naïve patients (FIG. 56G). Bars represent the mean, t-line the s.e.m., and symbols represent individual data points (* P≤0.05; ** P≤0.01; n=7, 8 and 12 biopsies for responders, treatment naïve and nonresponders, respectively). (FIG. 54B) Contour plots demonstrate the expression of TIM-3 and PD-1 in the TRM cells isolated from pre-immunotherapy biopsies (gated as per FIG. 54A). (FIG. 54C) Singlecell RNA-seq analysis of transcripts (one per row) differentially expressed between CTLs pre- and post-anti-PD-1 (MAST analysis), with an adjusted P value of <0.05), presented as row-wise z-scores of TPM counts; each column represents a single cell (n=127 and 151 cells, respectively). (FIG. 54D) Violin plot of expression of indicated transcripts differentially expressed between tumor-infiltrating CTLs isolated from pre- and post-anti-PD-1 treatment samples (as per FIG. 54C); shape represents the distribution of expression among cells and color represents average expression, calculated from the TPM counts. (FIG. 54E) GSEA of the bulk tumor CD103+ versus. CD103− transcriptional signature (FIG. 3a) and TIM-3+IL7R− TRM cell 29 signature (FIG) in tumor-infiltrating CTLs isolated from pre- and post-anti-PD-1 treatment samples: top, running enrichment score (RES) for the gene set, from most enriched at the left to most under-represented at the right; middle, positions of gene set members (blue vertical lines) in the ranked list of genes; bottom, value of the ranking metric. Values above the plot represent the normalized enrichment score (NES) and FDR-corrected significance. (FIG. 54F) Spearman co-expression analysis of transcripts enriched in tumor-infiltrating CTLs from post-anti-PD-1 treatment samples (c); matrix is clustered according to complete linkage. (FIG. 54G) Correlation analysis of all peaks identified in the OMNI-ATAC-seq libraries, pooled from 9 donors across two experiments, cells were sorted on CD14−CD19−CD20−CD4−CD45+CD3+CD8+CD103+TIM-3+IL-7R− and CD14−CD19−CD20−CD4−CD45+CD3+CD8+CD103−. Matrix is clustered according to complete linkage. (FIG. 54H) University of California Santa Cruz genome browser tracks for key TRM-associated gene loci as indicated above the tracks. RNA-seq tracks are merged from all purified bulk RNA-seq data, presented as Reads Per Kilobase Million (RPKM) (as per FIG. 2B; tumor non-TRM=25, tumor TRM=19; OMNI-ATACseq as per FIG. 54G).

FIGS. 55A-55C: Validation of T_{RM} phenotype. (FIG. 55A) Flow-cytometry contour plots showing the expression of CD49A and KLRG1 versus. that of CD103 in live, singlet, CD14−CD19−CD20−CD4−CD45+CD3+CD8+ cells obtained from lung samples (n=6). (FIG. 55B) GSEA of the murine composite TRM signature in the transcriptome of TRM versus. non-TRM: top, running enrichment score (RES) for the gene set, from most enriched genes at left to most underrepresented at right; middle, positions of gene set members (blue vertical lines) in the ranked list of genes; bottom, value of the ranking metric. Values above the plot represent the normalized enrichment score (NES) and false discovery rate (FDR)-corrected significance value in CTLs isolated from lung and tumor samples. (FIG. 55C) GSEA of the lung TRM versus. non-TRM cells for non-preserved transcripts (in FIG. 1E, FIG. 1F; as per e; N/S=Not significant).

FIGS. 56A-56B: PD-1 is co-expressed with cytotoxicity associated molecules at the protein level ex-vivo. (FIG. 56A) Flow-cytometry analysis of PD-1+ TRM and non-TRM cells versus. a particular cytokine (as indicated below the plots) following 4 hours of ex-vivo stimulation. Gated on live and singlet-gated CD14−CD20−CD4−CD45+CD3+CD8+ cells obtained from lung cancer TILs (FIG. 56B) Analysis of Granzyme A and Granzyme B directly exvivo, Gated and analyzed, as per (FIG. 56A).

FIG. 57: TIM-3+IL7R− TRM cells are enriched in responders to anti-PD-1 therapy. Flow-cytometry analysis of the expression of TIM-3 versus. that of IL-7R in live, singlet CD14CD20−CD4−CD45+CD3+CD8+CD103+ cells obtained from patients responding or not-responding to anti-PD-1 therapy (n=18).

FIGS. 58A-58D: Single-cell transcriptome analysis of CTLs from anti-PD-1 responders. (FIG. 58A) Schematic representation of clinical details and cells sorted for the patients selected for study (time point—TP). (FIG. 58B) Example of in-silico removal of CD4+ cells, highlighting the transcriptomic drop outs. The dashed line corresponds to the CD4+ cells removed. (FIG. 58C) A clonotype network graph of cells from (FIG. 58A), highlighting the time point from which the cells were isolated. Cells highlighted through a dashed line correspond to shared clonotypes across time points. (FIG. 58D) A clonotype network graph (as per c), highlighting the TRM cells and non-TRM cells, marked respectively. Cells were assigned based on protein expression of CD103, alternatively if cell-specific protein expression was not available, cells with greater than 10 TPM counts expression of either ITGAE (CD103), RBPJ or ZNF683 (HOBIT) considered a TRM.

### TABLES

Table 1. List of prioritized genes
Table 2. Expanded list of prioritized genes
Table 3. List of differentially expressed genes in Lung TRM from non-TRM
Table 4. List of differentially expressed genes in tumor TRM from tumor non-TRM
Table 5. List of uniquely expressed genes in tumor TRM
Table 6. TCR-seq library and clonality information
Table 7. List of uniquely expressed genes in tumor TRM subtypes

### DETAILED DESCRIPTION

It is to be understood that the present disclosure is not limited to particular aspects described, as such may, of course, vary. It is also to be understood that the terminology used herein is for the purpose of describing particular embodiments only, and is not intended to be limiting, since the scope of the present disclosure will be limited only by the appended claims.

A number of embodiments of the disclosure have been described. Nevertheless, it will be understood that various modifications may be made without departing from the spirit and scope of the disclosure. Accordingly, the following examples are intended to illustrate but not limit the scope of disclosure described in the claims.

It is to be inferred without explicit recitation and unless otherwise intended, that when the present technology relates to a polypeptide, protein, polynucleotide or antibody, an equivalent or a biologically equivalent of such is intended within the scope of the present technology.

Throughout this disclosure, various publications, patents and published patent specifications are referenced by an identifying citation. The full bibliographic information for the citations is found immediately preceding the claims. All publications, patent applications, patents, and other references mentioned herein are expressly incorporated by reference in their entirety, to the same extent as if each were incorporated by reference individually. In case of conflict, the present specification, including definitions, will control.

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The disclosure is generally disclosed herein using affirmative language to describe the numerous embodiments and aspects. The disclosure also specifically includes embodiments in which particular subject matter is excluded, in full or in part, such as substances or materials, method steps and conditions, protocols, or procedures. For example, in certain embodiments or aspects of the disclosure, materials and/or method steps are excluded. Thus, even though the disclosure is generally not expressed herein in terms of what the disclosure does not include aspects that are not expressly excluded in the disclosure are nevertheless disclosed herein.

The technology illustratively described herein suitably can be practiced in the absence of any element(s) not specifically disclosed herein. Thus, for example, in each instance herein any of the terms “comprising,” “consisting essentially of,” and “consisting of” can be replaced with either of the other two terms. The terms and expressions which have been employed are used as terms of description and not of limitation and use of such terms and expressions do not exclude any equivalents of the features shown and described or segments thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term “substantially” as used herein refers to a value modifier meaning “at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

Thus, it should be understood that although the present technology has been specifically disclosed by representative embodiments and optional features, modification and variation of the concepts herein disclosed can be resorted to by those skilled in the art, and such modifications and variations are considered within the scope of this technology.

### Definitions

As used in the specification and claims, the singular form “a”, “an”, and “the” include plural references unless the context clearly dictates otherwise. For example, the term “a cell” includes a plurality of cells, including mixtures thereof.

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Embodiments defined by each of these transition terms are within the scope of this disclosure.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

As used herein, the term “animal” refers to living multi-cellular vertebrate organisms, a category that includes, for example, mammals and birds. The term “mammal” includes both human and non-human mammals, e.g., bovines, canines, felines, rat, murines, simians, equines and humans. Additional examples include adults, juveniles and infants

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

As used herein “a population of cells” intends a collection of more than one cell that is identical (clonal) or non-identical in phenotype and/or genotype.

As used herein, “substantially homogenous” population of cells is a population having at least 70%, or alternatively at least 75%, or alternatively at least 80%, or alternatively at least 85%, or alternatively at least 90%, or alternatively at least 95%, or alternatively at least 98% identical phenotype, as measured by pre-selected markers, phenotypic or genomic traits. In one aspect, the population is a clonal population.

As used herein, “heterogeneous” population of cells is a population having up to 69%, or alternatively up to 60%, or alternatively up to 50%, or alternatively up to 40%, or alternatively up to 30%, or alternatively up to 20%, or alternatively up to 10%, or alternatively up to 5%, or alternatively up to 4%, or alternatively up to 3%, or alternatively up to 2%, or alternatively up to 61%, or alternatively up to 0.5% identical phenotype, as measured by pre-selected markers, phenotypic or genomic traits.

A “composition” typically intends a combination of the active agent, e.g., an engineered immune cell, e.g. T-cell, a modified T-cell, a NK cell, a chimeric antigen cell, a cell comprising an engineered immune cell, e.g. a T-cell, a NK cell, a CART cell or a CAR NK cell, an antibody, a cytokine, IL-12, a compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term siRNA intends short hairpin RNAs (shRNAs). shRNAs comprise a single strand of RNA that forms a stem-loop structure, where the stem consists of the complementary sense and antisense strands that comprise a double-stranded siRNA, and the loop is a linker of varying size. The stem structure of shRNAs generally is from about 10 to about 30 nucleotides long.

The term microRNAs (miRNAs) intends a class of small noncoding RNAs of about 22 nucleotide in length which are involved in the regulation of gene expression at the posttranscriptional level by degrading their target mRNAs and/or inhibiting their translation.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “isolated cell” generally refers to a cell that is substantially separated from other cells of a tissue. The term includes prokaryotic and eukaryotic cells.

“Immune cells” includes, e.g., white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Cytokines are small secreted proteins released by immune cells that have a specific effect on the interactions and communications between the immune cells. Cytokines can be pro-inflammatory or anti-inflammatory. Non-limiting example of a cytokine is Granulocyte-macrophage colony-stimulating factor (GM-CSF), which stimulates stem cells to produce granulocytes (neutrophils, eosinophils, and basophils) and monocytes.

As used herein, the phrase “immune response” or its equivalent “immunological response” or “anti-tumor response” refers to the development of a cell-mediated response (e.g. mediated by antigen-specific T cells or their secretion products). A cellular immune response is elicited by the presentation of polypeptide epitopes in association with Class I or Class II MHC molecules, to treat or prevent a viral infection, expand antigen-specific B-reg cells, TC1, CD4+T helper cells and/or CD8+ cytotoxic T cells and/or disease generated, autoregulatory T cell and B cell “memory” cells. The response may also involve activation of other components. In some aspect, the term “immune response” may be used to encompass the formation of a regulatory network of immune cells. Thus, the term “regulatory network formation” may refer to an immune response elicited such that an immune cell, preferably a T cell, more preferably a T regulatory cell, triggers further differentiation of other immune cells, such as but not limited to, B cells or antigen-presenting cells—non-limiting examples of which include dendritic cells, monocytes, and macrophages. In certain embodiments, regulatory network formation involves B cells being differentiated into regulatory B cells; in certain embodiments, regulatory network formation involves the formation of tolerogenic antigen-presenting cells.

The term “transduce” or “transduction” as it is applied to the production of chimeric antigen receptor cells refers to the process whereby a foreign nucleotide sequence is introduced into a cell. In some embodiments, this transduction is done via a vector.

As used herein, the term “vector” refers to a nucleic acid construct deigned for transfer between different hosts, including but not limited to a plasmid, a virus, a cosmid, a phage, a BAC, a YAC, etc. A “viral vector” is defined as a recombinantly produced virus or viral particle that comprises a polynucleotide to be delivered into a host cell, either in vivo, ex vivo or in vitro. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. Further details as to modern methods of vectors for use in gene transfer may be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.).

An “an effective amount” or “efficacious amount” is an amount sufficient to achieve the intended purpose, non-limiting examples of such include: initiation of the immune response, modulation of the immune response, suppression of an inflammatory response and modulation of T cell activity or T cell populations. In one aspect, the effective amount is one that functions to achieve a stated therapeutic purpose, e.g., a therapeutically effective amount. As described herein in detail, the effective amount, or dosage, depends on the purpose and the composition, and can be determined according to the present disclosure.

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg), Tissue-resident memory T cells (Tim cells) and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4;11 (ATCC CRL-1873), CCRF-CEM (ATCC CRM-CCL-119); and cutaneous T-cell lymphoma lines, e.g., HuT78 (ATCC CRM-TIB-161), MJ[G11] (ATCC CRL-8294), HuT102 (ATCC TIB-162). Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, the term “engineered T-cell receptor” refers to a molecule comprising the elements of (a) an extracellular antigen binding domain, (b) a transmembrane domain, and (c) an intracellular signaling domain. In some aspect, an engineered T-cell receptor is a genetically modified TCR, a modified TCR, a recombinant TCR, a transgenic TCR, a partial TCR, a chimeric fusion protein, a CAR, a first generation CAR, a second generation CAR, a third generation CAR, or a fourth generation TRUCK. In some aspect, the engineered T-cell receptor comprises an antibody or a fragment of an antibody. In particular aspects, the engineered T-cell receptor is a genetically modified TCR or a CAR.

As used herein, the term “receptor” or “T-cell receptor” or “TCR” refers to a cell surface molecule found on T-cells that functions to recognize and bind antigens presented by antigen presenting molecules. Generally, a TCR is a heterodimer of an alpha chain (TRA) and a beta chain (TRB). Some TCRs are comprised of alternative gamma (TRG) and delta (TRD) chains. T-cells expressing this version of a TCR are known as γδ T-cells. TCRs are part of the immunoglobulin superfamily. Accordingly, like an antibody, the TCR comprises three hypervariable CDR regions per chain There is also an additional area of hypervariability on the beta-chain (HV4). The TCR heterodimer is generally present in an octomeric complex that further comprises three dimeric signaling modules CD3γ/ε, CD3δ/ε, and CD247 ζ/ζ or ζ/η. Non-limiting exemplary amino acid sequence of the human TCR-alpha chain: METLLGVSLVILWLQLARVNSQQGEEDPQALSIQEGENATMNCS YKTSINNLQWYRQNSGRGLVHLILIRSNEREKHSGRLRVTLDTSKKSSSLLITASRA A DTASYFCAPVLSGGGADGLTFGKGTHLIIQPYIQNPDPAVYQLRDSKSSDKSVCLFT D FDSQTNVSQSKDSDVYITDKTVLDMRSMDFKSNSAVAWSNKSDFACANAFNNSIIP EDTFFPSPESSCDVKLVEKSFETDTNLNFQNLSVIGFRILLLKVAGFNLLMTLRLWSS

Non-limiting exemplary amino acid sequence of the human TCR-beta chain:

DSAVYLCASSLLRVYEQYFGPGTRLTVTEDLKNVFPPEVAVFEPPEAEI
SHTQKATLVCLATGFYPDHVELSWWVNGKEVHSGVSTDPQPLKEQP.

The term “modified TCR” refers to a TCR that has been genetically engineered, and/or a transgenic TCR, and/or a recombinant TCR. Non-limiting examples of modified TCRs include single-chain VαVβ TCRs (scTv), full-length TCRs produced through use of a T cell display system, and TCRs wherein the CDR regions have been engineered to recognize a specific antigen, peptide, fragment, and/or MHC molecule. Methods of developing and engineering modified TCRs are known in the art. For example, see Stone, J. D. et al. Methods in Enzymology 503: 189-222 (2012), PCT Application WO2014018863 A1.

As used herein, the term “antibody” (“Ab”) collectively refers to immunoglobulins (or “Ig”) or immunoglobulin-like molecules including but not limited to antibodies of the following isotypes: IgM, IgA, IgD, IgE, IgG, and combinations thereof. Immunoglobulin-like molecules include but are not limited to similar molecules produced during an immune response in a vertebrate, for example, in mammals such as humans, rats, goats, rabbits and mice, as well as non-mammalian species, such as shark immunoglobulins (see Feige, M. et al. Proc. Nat. Ac. Sci. 41(22): 8155-60 (2014)). Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 103 M−1 greater, at least 104 M−1 greater or at least 105 M−1 greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3rd Ed., W.H. Freeman & Co., New York, 1997.

As used herein, the term “monoclonal antibody” refers to an antibody produced by a cell into which the light and heavy chain genes of a single antibody have been transfected or, more traditionally, by a single clone of B-lymphocytes. Monoclonal antibodies generally have affinity for a single epitope (i.e. they are monovalent) but may be engineered to be specific for two or more epitopes (e.g. bispecific). Methods of producing monoclonal antibodies are known to those of skill in the art, for example by creating a hybridoma through fusion of myeloma cells with immune spleen cells, phage display, single cell amplification from B-cell populations, single plasma cell interrogation technologies, and single B-cell culture. Monoclonal antibodies include recombinant antibodies, chimeric antibodies, humanized antibodies, and human antibodies.

The general structure of an antibody is comprised of heavy (H) chains and light (L) chains connected by disulfide bonds. The structure can also comprise glycans attached at conserved amino acid residues. Each heavy and light chain contains a constant region and a variable region (also known as “domains”). There are two types of light chain, lambda (2) and kappa (κ). There are five primary types of heavy chains which determine the isotype (or class) of an antibody molecule: gamma (γ), delta (δ), alpha (α), mu (μ) and epsilon (ε). The constant regions of the heavy chain also contribute to the effector function of the antibody molecule. Antibodies comprising the heavy chains μ, δ, γ3, γ1, α1, γ2, γ4, ε, and α2 result in the following isotypes: IgM, IgD, IgG3, IgG1, IgA1, IgG2, IgG4, IgE, and IgA2, respectively. An IgY isotype, related to mammalian IgG, is found in reptiles and birds. An IgW isotype, related to mammalian IgD, is found in cartilaginous fish. Class switching is the process by which the constant region of an immunoglobulin heavy chain is replaced with a different immunoglobulin heavy chain through recombination of the heavy chain locus of a B-cell to produce an antibody of a different isotype. Antibodies may exist as monomers (e.g. IgG), dimers (e.g. IgA), tetramers (e.g. fish IgM), pentamers (e.g. mammalian IgM), and/or in complexes with other molecules. In some embodiments, antibodies can be bound to the surface of a cell or secreted by a cell.

The variable regions of the immunoglobulin heavy and the light chains specifically bind the antigen. The “framework” region is a portion of the Fab that acts as a scaffold for three hypervariable regions called “complementarity-determining regions” (CDRs). A set of CDRs is known as a paratope. The framework regions of different light or heavy chains are relatively conserved within a species. The combined framework region of an antibody (comprising regions from both light and heavy chains), largely adopts a β-sheet conformation and the CDRs form loops which connect, and in some cases form part of, the β-sheet structure. Thus, framework regions act to position the CDRs in correct orientation by inter-chain, non-covalent interactions. The framework region and CDRs for numerous antibodies have been defined and are available in a database maintained online (Kabat et al., Sequences of Proteins of Immunological Interest, U.S. Department of Health and Human Services, 1991).

The CDRs of the variable regions of heavy and light chains (VH and VL) are responsible for binding to an epitope of an antigen. A limited number of amino acid positions within the CDRs are directly involved in antigen binding. These positions within the CDRs are called specificity determining residues (SDRs). The CDRs of a heavy or light chain are numbered sequentially starting from the N-terminal end (i.e. CDR1, CDR2, and CDR3). For example, a VL CDR3 is the middle CDR located in the variable domain of the light chain of an antibody. A VH CDR1 is the first CDR in the variable domain of a heavy chain of an antibody. An antibody that binds a specific antigen will have specific VH and VL region sequences, and thus specific CDR sequences. Antibodies with different specificities (i.e. different combining sites for different antigens) have different CDRs.

The term “humanized” when used in reference to an antibody, means that the amino acid sequence of the antibody has non-human amino acid residues (e.g., mouse, rat, goat, rabbit, etc.) of one or more complementarity determining regions (CDRs) that specifically bind to the desired antigen in an acceptor human immunoglobulin molecule, and one or more human amino acid residues in the Fv framework region (FR), which are amino acid residues that flank the CDRs. Such antibodies typically have reduced immunogenicity and therefore a longer half-life in humans as compared to the non-human parent antibody from which one or more CDRs were obtained or are based upon.

An “antigen-binding fragment” (Fab) refers to the regions of an antibody corresponding to two of the three fragments produced by papain digestion. The Fab fragment comprises the region that binds to an antigen and is composed of one variable region and one constant region from both a heavy chain and a light chain. An F(ab′)2 fragment refers to a fragment of an antibody digested by pepsin or the enzyme IdeS (immunoglobulin degrading enzyme from S. pyogenes) comprising two Fab regions connected by disulfide bonds. A single chain variable fragment (“scFv”) refers to a fusion protein comprising at least one VH and at least one VL region connected by a linker of between 5 to 30 amino acids. Methods and techniques of developing scFv that bind to specific antigens are known in the art (see, e.g. Ahmad, Z. A. et al., Clinical and Developmental Immunology, 2012: 980250 (2012)).

As used herein, the term “antigen” refers to a compound, composition, or substance that may be specifically bound and/or recognized by the products of specific humoral or cellular immunity and antigen recognition molecules, including but not limited to an antibody molecule, single-chain variable fragment (scFv), cell surface immunoglobulin receptor, B-cell receptor (BCR), T-cell receptor (TCR), engineered TCR, modified TCR, or CAR. The term “epitope” refers to an antigen or a fragment, region, site, or domain of an antigen that is recognized by an antigen recognition molecule. Antigens can be any type of molecule including but not limited to peptides, proteins, lipids, phospholipids haptens, simple intermediary metabolites, sugars (e.g., monosaccharides or oligosaccharides), hormones, and macromolecules such as complex carbo-hydrates (e.g., polysaccharides). Some non-limiting examples of antigens include antigens involved in autoimmune disease (including autoantigens), allergy, and graft rejection, tumor antigens, toxins, and other miscellaneous antigens. Non-limiting examples of tumor antigens include mesothelin, ROR1 and EGFRvIII, ephrin type-A receptor 2 (EphA2), interleukin (IL)-13r alpha 2, an EGFR VIII, a PSMA, an EpCAM, a GD3, a fucosyl GM1, a PSCA, a PLAC1, a sarcoma breakpoint, a Wilms Tumor 1, a hematologic differentiation antigen, a surface glycoprotein, a gangliosides (GM2), a growth factor receptor, a stromal antigen, a vascular antigen, or a combination thereof. Antigens expressed by pathogens include, but are not limited to microbial antigens such as viral antigens, bacterial antigens, fungal antigens, protozoa, and other parasitic antigens.

As used herein, the term “target cell population” refers to a population of cells that present antigens, which can be targeted by engineered T cells. Non-limiting examples of target cell populations include tumor cells, cancer cells and pathogen infected cells. Non-limiting examples of pathogens include viral and bacterial pathogens.

As used herein, the term “antigen binding domain” refers to any protein or polypeptide domain that can specifically bind to an antigen target (including target complexes of antigens and MHC molecules).

As used herein, the term “autologous,” in reference to cells, tissue, and/or grafts refers to cells, tissue, and/or grafts that are isolated from and then and administered back into the same subject, patient, recipient, and/or host. “Allogeneic” refers to non-autologous cells, tissue, and/or grafts.

As used herein, the term “B cell,” refers to a type of lymphocyte in the humoral immunity of the adaptive immune system. B cells principally function to make antibodies, serve as antigen presenting cells, release cytokines, and develop memory B cells after activation by antigen interaction. B cells are distinguished from other lymphocytes, such as T cells, by the presence of a B-cell receptor on the cell surface. B cells may either be isolated or obtained from a commercially available source. Non-limiting examples of commercially available B cell lines include lines AHH-1 (ATCC® CRL-8146™), BC-1 (ATCC® CRL-2230™), BC-2 (ATCC® CRL-2231™), BC-3 (ATCC® CRL-2277™), CA46 (ATCC® CRL-1648™), DG-75 [D.G.-75] (ATCC® CRL-2625™), DS-1 (ATCC® CRL-11102™), EB-3 [EB3] (ATCC® CCL-85™), Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), AND SUP-B15 (ATCC CRL-1929). Further examples include but are not limited to cell lines derived from anaplastic and large cell lymphomas, e.g., DEL, DL-40, FE-PD, JB6, Karpas 299, Ki-JK, Mac-2A Ply1, SR-786, SU-DHL-1, -2, -4, -5, -6, -7, -8, -9, -10, and -16, DOHH-2, NU-DHL-1, U-937, Granda 519, USC-DHL-1, RL; Hodgkin's lymphomas, e.g., DEV, HDLM-2, HD-MyZ, KM-H2, L 428, L 540, L1236, SBH-1, SUP-HD1, SU/RH-HD-1. Non-limiting exemplary sources for such commercially available cell lines include the American Type Culture Collection, or ATCC, (www.atcc.org/) and the German Collection of Microorganisms and Cell Cultures (https://www.dsmz.de/).

As used herein, the term “major histocompatibility complex” (MHC) refers to an antigen presentation molecule that functions as part of the immune system to bind antigens and other peptide fragments and display them on the cell surface for recognition by antigen recognition molecules such as TCR. MHC may be used interchangeably with the term “human leukocyte antigen” (HLA) when used in reference to human MHC; thus, MHC refers to all HLA subtypes including, but not limited to, the classical MHC genes disclosed herein: HLA-A, HLA-E, HLA-DM, HLA-DO, HLA-DP, HLA-DQ, and HLA-DR, in addition to all variants, isoforms, isotypes, and other biological equivalents thereof. MHC class I (MHC-I) and MHC class II (MHC-II) molecules utilize distinct antigen processing pathways. In general, peptides derived from intracellular antigens are presented to CD8+ T cells by MHC class I molecules, which are expressed on virtually all cells, while extracellular antigen-derived peptides are presented to CD4+ T cells by MHC-II molecules. However, several exceptions to this dichotomy have been observed. In certain embodiments disclosed herein, a particular antigen, peptide, and/or epitope is identified and presented in an antigen-MHC complex in the context of an appropriate MHC class I or II protein. The genetic makeup of a subject may be assessed to determine which MHC allele is suitable for a particular patient, disease, or condition with a particular set of antigens. In mice, the MHC genes are known as the histocompatibility 2 (H-2) genes. Murine classical MHC class I subtypes include H-2D, H-2K, and H-2L. Murine non-classical MHC class I subtypes include H-2Q, H-2M, and H-2T. Murine classical MHC class II subtypes include H-2A (I-A), and H-2E (1-E). Non-classical murine MHC class II subtypes include H-2M and H-20. Canine MHC molecules are known as Dog Leukocyte Antigens (DLA). Feline MHC molecules are known as Feline Leukocyte Antigens (FLA). In some embodiments, an orthologous or homologous MHC molecule is selected to transition a therapy or treatment involving a specific antigen-MHC complex from one species to a different species.

As used herein, a “target cell” is any cell that expresses the antigen target to which the engineered T cells can bind.

As used herein, a “cancer” is a disease state characterized by the presence in a subject of cells demonstrating abnormal uncontrolled replication and may be used interchangeably with the term “tumor.” In some embodiments, the cancer is a leukemia or a lymphoma. “Cell associated with the cancer” refers to those subject cells that demonstrate abnormal uncontrolled replication. In certain embodiments, the cancer is acute myeloid leukemia or acute lymphoblastic leukemia. As used herein a “leukemia” is a cancer of the blood or bone marrow characterized by an abnormal increase of immature white blood cells. The specific condition of acute myeloid leukemia (AML)—also referred to as acute myelogenous leukemia or acute myeloblastic leukemia—is a cancer of the myeloid origin blood cells, characterized by the rapid growth of abnormal myeloid cells that accumulate in the bone marrow and interfere with the production of normal blood cells. The specific condition of acute lymphoblastic leukemia (ALL)—also referred to as acute lymphocytic leukemia or acute lymphoid leukemia—is a cancer of the white blood cells, characterized by the overproduction and accumulation of malignant, immature leukocytes (lymphoblasts) resulting a lack of normal, healthy blood cells. As used herein a “lymphoma” is a cancer of the blood characterized by the development of blood cell tumors and symptoms of enlarged lymph nodes, fever, drenching sweats, unintended weight loss, itching, and constantly feeling tired.

A “solid tumor” is an abnormal mass of tissue that usually does not contain cysts or liquid areas. Solid tumors can be benign or malignant. Different types of solid tumors are named for the type of cells that form them. Examples of solid tumors include sarcomas, carcinomas, and lymphomas.

The term “B-cell lymphoma or leukemia” refers to a type of cancer that forms in issues of the lymphatic system or bone marrow and has undergone a malignant transformation that makes the cells within the cancer pathological to the host organism with the ability to invade or spread to other parts of the body.

One of skill in the art can monitor expression of genes using methods such as RNA-sequencing, DNA microarrays, Real-time PCR, or Chromatin immunoprecipitation (ChIP) etc. Protein expression can be monitored using methods such as flow cytometry, Western blotting, 2-D gel electrophoresis or immunoassays etc.

One of skill in the art can use methods such as RNA interference (RNAi), CRISPR, TALEN, ZFN or other methods that target specific sequences to reduce or eliminate expression and/or function of proteins. CRISPR, TALEN, ZFN or other genome editing tools can also be used to increase expression and/or function of genes.

As used herein, “RNAi” (RNA interference) refers to the method of reducing or eliminating gene expression in a cell by targeting specific mRNA sequences for degradation via introduction of short pieces of double stranded RNA (dsRNA) and small interfering RNA (such as siRNA, shRNA or miRNA etc.) (Agrawal, N. et al.; Microbiol Mol Biol Rev. 2003; 67:657-685, Arenz, C. et al.; Naturwissenschaften. 2003; 90:345-359, Hannon G J.; Nature. 2002; 418:244-251).

As used herein, the term “CRISPR” refers to a technique of sequence specific genetic manipulation relying on the clustered regularly interspaced short palindromic repeats pathway. CRISPR can be used to perform gene editing and/or gene regulation, as well as to simply target proteins to a specific genomic location. “Gene editing” refers to a type of genetic engineering in which the nucleotide sequence of a target polynucleotide is changed through introduction of deletions, insertions, single stranded or double stranded breaks, or base substitutions to the polynucleotide sequence. In some aspects, CRISPR-mediated gene editing utilizes the pathways of non-homologous end joining (NHEJ) or homologous recombination to perform the edits. Gene regulation refers to increasing or decreasing the production of specific gene products such as protein or RNA.

The term “gRNA” or “guide RNA” as used herein refers to guide RNA sequences used to target specific polynucleotide sequences for gene editing employing the CRISPR technique. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. (2016) J of Biotechnology 233 (2016) 74-83).

The term “Cas9” refers to a CRISPR associated endonuclease referred to by this name. Non-limiting exemplary Cas9s include Staphylococcus aureus Cas9, nuclease dead Cas9, and orthologs and biological equivalents each thereof. Orthologs include but are not limited to Streptococcus pyogenes Cas9 (“spCas9”), Cas 9 from Streptococcus thermophiles, Legionella pneumophilia, Neisseria lactamica, Neisseria meningitides, Francisella novicida; and Cpfl (which performs cutting functions analogous to Cas9) from various bacterial species including Acidaminococcus spp. and Francisella novicida U112.

As used herein, “TALEN” (transcription activator-like effector nucleases) refers to engineered nucleases that comprise a non-specific DNA-cleaving nuclease fused to a TALE DNA-binding domain, which can target DNA sequences and be used for genome editing. Boch (2011) Nature Biotech. 29: 135-6; and Boch et al. (2009) Science 326: 1509-12; Moscou et al. (2009) Science 326: 3501. TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence. To produce a TALEN, a TALE protein is fused to a nuclease (N), which is a wild-type or mutated Fokl endonuclease. Several mutations to Fokl have been made for its use in TALENs; these, for example, improve cleavage specificity or activity. Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Bio. 200: 96. The Fokl domain functions as a dimer, requiring two constructs with unique DNA binding domains for sites in the target genome with proper orientation and spacing. Both the number of amino acid residues between the TALE DNA binding domain and the Fokl cleavage domain and the number of bases between the two individual TALEN binding sites appear to be important parameters for achieving high levels of activity. Miller et al. (2011) Nature Biotech. 29: 143-8. TALENs specific to sequences in immune cells can be constructed using any method known in the art, including various schemes using modular components. Zhang et al. (2011) Nature Biotech. 29: 149-53; Geibler et al. (2011) PLoS ONE 6: e 19509.

As used herein, “ZFN” (Zinc Finger Nuclease) refers to engineered nucleases that comprise a non-specific DNA-cleaving nuclease fused to a zinc finger DNA binding domain, which can target DNA sequences and be used for genome editing. Like a TALEN, a ZFN comprises a Fokl nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160. A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Like a TALEN, a ZFN must dimerize to cleave DNA. Thus, a pair of ZFNs are required to target non-palindromic DNA sites. The two individual ZFNs must bind opposite strands of the DNA with their nucleases properly spaced apart. Bitinaite et al. (1998) Proc. Natl. Acad. Sci. USA 95: 10570-5. ZFNs specific to sequences in immune cells can be constructed using any method known in the art. See, e.g., Provasi (2011) Nature Med. 18: 807-815; Torikai (2013) Blood 122: 1341-1349; Cathomen et al. (2008) Mol. Ther. 16: 1200-7; Guo et al. (2010) J. Mol. Biol. 400: 96; U.S. Patent Publication 201110158957; and U.S. Patent Publication 2012/0060230.

A “cytotoxic cell” intends a cell that is capable of killing other cells or microbes. Examples of cytotoxic cells include but are not limited to CD8+ T cells, natural-killer (NK) cells, NKT cells, and neutrophils, which cells are capable of mediating cytotoxicity responses.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, (3-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I.

As used herein, the term “purification marker” or “reporter protein” refer to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding the chimeric PVX described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical,” percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

It is to be inferred without explicit recitation and unless otherwise intended, that when the present disclosure relates to a polypeptide, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide or nucleic acid, intends those having minimal homology while still maintaining desired structure or functionality. Unless specifically recited herein, it is contemplated that any of the above also includes equivalents thereof. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and/or exhibits substantially equivalent biological activity to the reference protein, polypeptide, or nucleic acid. Alternatively, when referring to polynucleotides, an equivalent thereof is a polynucleotide that hybridizes under stringent conditions to the reference polynucleotide or its complement.

The phrase “equivalent polypeptide” or “equivalent peptide fragment” refers to protein, polynucleotide, or peptide fragment encoded by a polynucleotide that hybridizes to a polynucleotide encoding the exemplified polypeptide or its complement of the polynucleotide encoding the exemplified polypeptide, under high stringency and/or which exhibit similar biological activity in vivo, e.g., approximately 100%, or alternatively, over 90% or alternatively over 85% or alternatively over 70%, as compared to the standard or control biological activity. Additional embodiments within the scope of this disclosure are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology. Percentage homology can be determined by sequence comparison using programs such as BLAST run under appropriate conditions. In one aspect, the program is run under default parameters.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

As used herein, “anti-tumor immunity” in a subject refers to reducing or preventing the symptoms or cancer from occurring in a subject that is predisposed or does not yet display symptoms of the cancer.

In some embodiments a subject is in need of a treatment, cell or composition described herein. In certain embodiments a subject has or is suspected of having a neoplastic disorder, neoplasia, tumor, malignancy or cancer. In some embodiments a subject in need of a treatment, cell or composition described herein has or is suspected of having a neoplastic disorder, neoplasia, tumor, malignancy or cancer. In certain embodiments an engineered T cell described herein is used to treat a subject having, or suspected of having, a neoplastic disorder, neoplasia, tumor, malignancy or cancer.

In some embodiments, presented herein is a method of treating a subject having or suspected of having, a neoplasia, neoplastic disorder, tumor, cancer, or malignancy. In certain embodiments, a method of treating a subject comprises administering a therapeutically effective amount of an engineered T cell to a subject. In certain embodiments, a method comprises reducing or inhibiting proliferation of a neoplastic cell, tumor, cancer or malignant cell, comprising contacting the cell, tumor, cancer or malignant cell, with the engineered T cell in an amount sufficient to reduce or inhibit proliferation of the neoplastic cell, tumor, cancer or malignant cell.

In some embodiments, a method of reducing or inhibiting metastasis of a neoplasia, tumor, cancer or malignancy to other sites, or formation or establishment of metastatic neoplasia, tumor, cancer or malignancy at other sites distal from a primary neoplasia, tumor, cancer or malignancy, comprises administering to a subject an amount of an engineered T cell sufficient to reduce or inhibit metastasis of the neoplasia, tumor, cancer or malignancy to other sites, or formation or establishment of metastatic neoplasia, tumor, cancer or malignancy at other sites distal from the primary neoplasia, tumor, cancer or malignancy.

Non-limiting examples of a neoplasia, neoplastic disorder, tumor, cancer or malignancy include a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, or melanoma. A neoplasia, neoplastic disorder, tumor, cancer or malignancy may comprise or involve hematopoietic cells. Non-limiting examples of a sarcoma include a lymphosarcoma, liposarcoma, osteosarcoma, chondrosarcoma, leiomyosarcoma, rhabdomyosarcoma or fibrosarcoma. In some embodiments, a neoplasia, neoplastic disorder, tumor, cancer or malignancy is a myeloma, lymphoma or leukemia. In some embodiments, a neoplasia, neoplastic disorder, tumor, cancer or malignancy comprises a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, tumor, or cancer. In some embodiments, a neoplasia, neoplastic disorder, tumor, cancer or malignancy comprises a small cell lung or non-small cell lung cancer. In some embodiments, a neoplasia, neoplastic disorder, tumor, cancer or malignancy comprises a stem cell neoplasia, tumor, cancer or malignancy. In some embodiments, a neoplasia, neoplastic disorder, tumor, cancer or malignancy.

In some embodiments, a method inhibits, or reduces relapse or progression of the neoplasia, neoplastic disorder, tumor, cancer or malignancy. In some embodiments, a method comprises administering an anti-cell proliferative, anti-neoplastic, anti-tumor, anti-cancer or immune-enhancing treatment or therapy. In some embodiments, a method of treatment results in partial or complete destruction of the neoplastic, tumor, cancer or malignant cell mass; a reduction in volume, size or numbers of cells of the neoplastic, tumor, cancer or malignant cell mass; stimulating, inducing or increasing neoplastic, tumor, cancer or malignant cell necrosis, lysis or apoptosis; reducing neoplasia, tumor, cancer or malignancy cell mass; inhibiting or preventing progression or an increase in neoplasia, tumor, cancer or malignancy volume, mass, size or cell numbers; or prolonging lifespan. In some embodiments, a method of treatment results in reducing or decreasing severity, duration or frequency of an adverse symptom or complication associated with or caused by the neoplasia, tumor, cancer or malignancy. In some embodiments, a method of treatment results in reducing or decreasing pain, discomfort, nausea, weakness or lethargy. In some embodiments, a method of treatment results in increased energy, appetite, improved mobility or psychological well-being.

As used herein, the term “administer” and “administering” are used to mean introducing the therapeutic agent (e.g. polynucleotide, vector, cell, modified cell, population) into a subject. The therapeutic administration of this substance serves to attenuate any symptom, or prevent additional symptoms from arising. When administration is for the purposes of preventing or reducing the likelihood of developing an autoimmune disease or disorder, the substance is provided in advance of any visible or detectable symptom. Routes of administration include, but are not limited to, oral (such as a tablet, capsule or suspension), topical, transdermal, intranasal, vaginal, rectal, subcutaneous intravenous, intraarterial, intramuscular, intraosseous, intraperitoneal, epidural and intrathecal.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As used herein, the term “gene expression profile” refers to measuring the expression level of multiple genes to establish an expression profile for a particular sample.

As used herein, the term “lower than baseline expression” refers to reducing or eliminating the transcription of polynucleotides into mRNA, or alternatively reducing or eliminating the translation of mRNA into peptides, polypeptides, or proteins, or reducing or eliminating the functioning of peptides, polypeptides, or proteins. In a non-limiting example, the transcription of polynucleotides into mRNA is reduced to at least half of the normalized mean gene expression found in wild type cells.

As used herein, the term “higher than baseline expression” refers to increasing the transcription of polynucleotides into mRNA, or alternatively increasing the translation of mRNA into peptides, polypeptides, or proteins, or increasing the functioning of peptides, polypeptides, or proteins. In a non-limiting example, the transcription of polynucleotides into mRNA is increased to at least twice of the normalized mean gene expression found in wild type cells.

As used herein, the term “reduce or eliminate expression and/or function of” refers to reducing or eliminating the transcription of the polynucleotides into mRNA, or alternatively reducing or eliminating the translation of the mRNA into peptides, polypeptides, or proteins, or reducing or eliminating the functioning of the peptides, polypeptides, or proteins. In a non-limiting example, the transcription of polynucleotides into mRNA is reduced to at least half of its normal level found in wild type cells.

As used herein, the term “increase expression of” refers to increasing the transcription of the polynucleotides into mRNA, or alternatively increasing the translation of the mRNA into peptides, polypeptides, or proteins, or increasing the functioning of the peptides, polypeptides, or proteins. In a non-limiting example, the transcription of polynucleotides into mRNA is increased to at least twice of its normal level found in wild type cells.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

As used herein, the term “enhancer”, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

As used herein, the term “binds” or “antibody binding” or “specific binding” means the contact between the antigen binding domain of an antibody, antibody fragment, CAR, TCR, engineered TCR, BCR, MHC, immunoglobulin-like molecule, scFv, CDR or other antigen presentation molecule and an antigen, epitope, or peptide with a binding affinity (KD) of less than 10⁻⁵ M. In some aspects, an antigen binding domain binds to both a complex of both an antigen and an MHC molecule. In some aspects, antigen binding domains bind with affinities of less than about 10⁻⁶ M, 10⁻⁷M, and preferably 10⁻⁸ M, 10⁻⁹ M, 10⁻¹⁰ M, 10⁻¹¹M, or 10⁻¹² M. In a particular aspect, specific binding refers to the binding of an antigen to an MHC molecule, or the binding of an antigen binding domain of an engineered T-cell receptor to an antigen or antigen-MHC complex.

The term “introduce” as applied to methods of producing modified cells such as chimeric antigen receptor cells refers to the process whereby a foreign (i.e. extrinsic or extracellular) agent is introduced into a host cell thereby producing a cell comprising the foreign agent. Methods of introducing nucleic acids include but are not limited to transduction, retroviral gene transfer, transfection, electroporation, transformation, viral infection, and other recombinant DNA techniques known in the art. In some embodiments, transduction is done via a vector (e.g., a viral vector). In some embodiments, transfection is done via a chemical carrier, DNA/liposome complex, or micelle (e.g., Lipofectamine (Invitrogen)). In some embodiments, viral infection is done via infecting the cells with a viral particle comprising the polynucleotide of interest (e.g., AAV). In some embodiments, introduction further comprises CRISPR mediated gene editing or Transcription activator-like effector nuclease (TALEN) mediated gene editing. Methods of introducing non-nucleic acid foreign agents (e.g., soluble factors, cytokines, proteins, peptides, enzymes, growth factors, signaling molecules, small molecule inhibitors) include but are not limited to culturing the cells in the presence of the foreign agent, contacting the cells with the agent, contacting the cells with a composition comprising the agent and an excipient, and contacting the cells with vesicles or viral particles comprising the agent.

In the context of a nucleic acid or amino acid sequence, the term “chimeric” intends that the sequence contains is comprised of at least one substituent unit (e.g. fragment, region, portion, domain, polynucleotide, or polypeptide) that is derived from, obtained or isolated from, or based upon other distinct physical or chemical entities. For example, a chimera of two or more different proteins may comprise the sequence of a variable region domain from an antibody fused to the transmembrane domain of a cell signaling molecule. In some aspect, a chimera intends that the sequence is comprised of sequences from at least two distinct species.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” or “intracellular signaling domain” means any oligopeptide or polypeptide known to function as a domain that transmits a signal to cause activation or inhibition of a biological process in a cell. In certain embodiments, the intracellular domain may comprise, alternatively consist essentially of, or yet further comprise one or more costimulatory signaling domains in addition to the primary signaling domain. The “transmembrane domain” means any oligopeptide or polypeptide known to span the cell membrane and that can function to link the extracellular and signaling domains. A chimeric antigen receptor may optionally comprise a “hinge domain” which serves as a linker between the extracellular and transmembrane domains. Non-limiting exemplary polynucleotide sequences that encode for components of each domain are disclosed herein, e.g.:

Hinge domain: IgG1 heavy chain hinge polynucleotide sequence:

CTCGAGCCCAAATCTTGTGACAAAACTCACACATGCCCACCGTGCCCG,

and optionally an equivalent thereof.

Transmembrane domain: CD28 transmembrane region polynucleotide sequence:

TTTTGGGTGCTGGTGGTGGTTGGTGGAGTCCTGGCTTGCTATAGCTTGCT AGTAACAGTGGCCTTTATTATTTTCTGGGTG, and optionally an equivalent thereof.

Intracellular domain: 4-1BB co-stimulatory signaling region polynucleotide sequence:

AAACGGGGCAGAAAGAAACTCCTGTATATATTCAAACAACCATTTATGA GACCAGTACAAACTACTCAAGAGGAAGATGGCTGTAGCTGCCGATTTCCAGAA GAAGAAGAAGGAGGATGTGAACTG, and optionally an equivalent thereof.

Intracellular domain: CD28 co-stimulatory signaling region polynucleotide sequence:

AGGAGTAAGAGGAGCAGGCTCCTGCACAGTGACTACATGAACATGACTC CCCGCCGCCCCGGGCCCACCCGCAAGCATTACCAGCCCTATGCCCCACCACGCG ACTTCGCAGCCTATCGCTCC, and optionally an equivalent thereof.

Intracellular domain: CD3 zeta signaling region polynucleotide sequence:

AGAGTGAAGTTCAGCAGGAGCGCAGACGCCCCCGCGTACCAGCAGGGC CAGAACCAGCTCTATAACGAGCTCAATCTAGGACGAAGAGAGGAGTACGATGT TTTGGACAAGAGACGTGGCCGGGACCCTGAGATGGGGGGAAAGCCGAGAAGG AAGAACCCTCAGGAAGGCCTGTACAATGAACTGCAGAAAGATAAGATGGCGGA GGCCTACAGTGAGATTGGGATGAAAGGCGAGCGCCGGAGGGGCAAGGGGCAC GATGGCCTTTACCAGGGTCTCAGTACAGCCACCAAGGACACCTACGACGCCCTT CACATGCAGGCCCTGCCCCCTCGCTAA, and optionally an equivalent thereof.

Non-limiting examples of CAR extracellular domains capable of binding to antigens are the anti-CD19 binding domain sequences that specifically bind CD19 antigen as disclosed in the US20140271635 application.

Further embodiments of each exemplary domain component include other proteins that have analogous biological function that share at least 70%, or alternatively at least 80% amino acid sequence identity, preferably 90% sequence identity, more preferably at least 95% sequence identity with the proteins encoded by the above disclosed nucleic acid sequences. Further, non-limiting examples of such domains are provided herein.

As used herein, the term “CD8α hinge domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α hinge domain sequence as shown herein. The example sequences of CD8 α hinge domain for human, mouse, and other species are provided in Pinto, R. D. et al. (2006) Vet. Immunol. Immunopathol. 110:169-177. The sequences associated with the CD8 α hinge domain are provided in Pinto, R. D. et al. (2006) Vet. Immunol. Immunopathol. 110:169-177. Non-limiting examples of such include:

Human CD8 alpha hinge domain amino acid sequence: PAKPTTTPAPRPPTPAPTIASQPLSLRPEACRPAAGGAVHTRGLDFACDIY, and optionally an equivalent thereof.

Mouse CD8 alpha hinge domain amino acid sequence: KVNSTTTKPVLRTPSPVHPTGTSQPQRPEDCRPRGSVKGTGLDFACDIY, and optionally an equivalent thereof.

Cat CD8 alpha hinge domain amino acid sequence: PVKPTTTPAPRPPTQAPITTSQRVSLRPGTCQPSAGSTVEASGLDLSCDIY, and optionally an equivalent thereof.

As used herein, the term “CD8 α transmembrane domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α transmembrane domain sequence as shown herein. The fragment sequences associated with the amino acid positions 183 to 203 of the human T-cell surface glycoprotein CD8 alpha chain (GenBank Accession No: NP_001759.3), or the amino acid positions 197 to 217 of the mouse T-cell surface glycoprotein CD8 alpha chain (GenBank Accession No: NP_001074579.1), and the amino acid positions 190 to 210 of the rat T-cell surface glycoprotein CD8 alpha chain (GenBank Accession No: NP_113726.1) provide additional example sequences of the CD8 α transmembrane domain. The sequences associated with each of the listed accession numbers are provided as follows:

Human CD8 alpha transmembrane domain amino acid sequence: IYIWAPLAGTCGVLLLSLVIT, and optionally an equivalent thereof.

Mouse CD8 alpha transmembrane domain amino acid sequence: IWAPLAGICVALLLSLIITLI, and optionally an equivalent thereof.

Rat CD8 alpha transmembrane domain amino acid sequence: IWAPLAGICAVLLLSLVITLI, and optionally an equivalent thereof.

As used herein, the term “CD28 transmembrane domain” refers to a specific protein fragment associated with this name and any other molecules that have analogous biological function that share at least 70%, or alternatively at least 80% amino acid sequence identity, at least 90% sequence identity, or alternatively at least 95% sequence identity with the CD28 transmembrane domain sequence as shown herein. The fragment sequences associated with the GenBank Accession Nos: XM_006712862.2 and XM_009444056.1 provide additional, non-limiting, example sequences of the CD28 transmembrane domain.

As used herein, the term “4-1BB costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the 4-1BB costimulatory signaling region sequence as shown herein. Non-limiting example sequences of the 4-1BB costimulatory signaling region are provided in U.S. Publication 20130266551A1 (filed as U.S. application Ser. No. 13/826,258), such as the exemplary sequence provided below and the sequence encoded by 4-1BB costimulatory signaling region amino acid sequence: KRGRKKLLYIFKQPFMRPVQTTQEEDGCSCRFPEEEEGGCEL, and optionally an equivalent thereof.

As used herein, the term “ICOS costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the ICOS costimulatory signaling region sequence as shown herein. Non-limiting example sequences of the ICOS costimulatory signaling region are provided in U.S. Patent Application Publication No. 2015/0017141A1 the exemplary polynucleotide sequence provided below.

ICOS costimulatory signaling region polynucleotide sequence: ACAAAAAAGA AGTATTCATC CAGTGTGCAC GACCCTAACG GTGAATACAT GTTCATGAGA GCAGTGAACA CAGCCAAAAA ATCCAGACTC ACAGATGTGA CCCTA, and optionally an equivalent thereof.

As used herein, the term “OX40 costimulatory signaling region” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the OX40 costimulatory signaling region sequence as shown herein. Non-limiting example sequences of the OX40 costimulatory signaling region are disclosed in U.S. Patent Application Publication No. 2012/20148552A1, and include the exemplary sequence provided below.

OX40 costimulatory signaling region polynucleotide sequence:

AGGGACCAG AGGCTGCCCC CCGATGCCCA CAAGCCCCCT GGGGGAGGCA GTTTCCGGAC CCCCATCCAA GAGGAGCAGG CCGACGCCCA CTCCACCCTG GCCAAGATC, and optionally an equivalent thereof.

As used herein, the term “CD28 costimulatory signaling region” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the CD28 costimulatory signaling region sequence shown herein. The example sequences CD28 costimulatory signaling domain are provided in U.S. Pat. No. 5,686,281; Geiger, T. L. et al. (2001) Blood 98: 2364-2371; Hombach, A. et al. (2001) J Immunol 167: 6123-6131; Maher, J. et al. (2002) Nat Biotechnol 20: 70-75; Haynes, N. M. et al. (2002) J Immunol. 169: 5780-5786 (2002); Haynes, N. M. et al. (2002) Blood 100: 3155-3163. A non-limiting example include the sequence encoded by:

CD28 amino acid sequence: MLRLLLALNL FPSIQVTGNK ILVKQSPMLV AYDNAVNLSC KYSYNLFSRE FRASLHKGLDSAVEVCVVYG NYSQQLQVYS KTGFNCDGKL GNESVTFYLQ NLYVNQTDIY FCKIEVMYPPPYLDNEKSNG TIIHVKGKHL CPSPLFPGPS KPFWVLVVVG GVLACYSLLVTVAFIIFWVR SKRSRLLHSD YMNMTPRRPG PTRKHYQPYA PPRDFAAYRS, and equivalents thereof.

As used herein, the term “CD3 zeta signaling domain” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the CD3 zeta signaling domain sequence as shown herein. Non-limiting example sequences of the CD3 zeta signaling domain amino acid sequence are provided in U.S. application Ser. No. 13/826,258, e.g.:

RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

As used herein, a “first generation CAR” refers to a CAR comprising an extracellular domain capable of binding to an antigen, a transmembrane domain derived from a polypeptide different from a polypeptide from which the extracellular domain is derived, and at least one intracellular domain. A “second generation CAR” refers to a first generation CAR further comprising one costimulation domain (e.g. 4-1BB or CD28). A “third generation CAR” refers to a first generation CAR further comprising two costimulation domains (e.g. CD27, CD28, ICOS, 4-1BB, or OX40). A “fourth generation CAR” (also known as a “TRUCK”) refers to a CAR T-cell further engineered to secrete an additional factor (e.g. proinflammatory cytokine IL-12). A review of these CAR technologies and cell therapy is found in Maus, M. et al. Clin. Cancer Res. 22(3): 1875-84 (2016).

As used herein, the term “suicide gene” is a gene capable of inducing cell apoptosis; non-limiting examples include HSV-TK (Herpes simplex virus thymidine kinase), cytosine deaminase, nitroreductase, carboxylesterase, cytochrome P450 or PNP (Purine nucleoside phosphorylase), truncated EGFR, or inducible caspase (“iCasp”). Suicide genes may function along a variety of pathways, and, in some cases, may be inducible by an inducing agent such as a small molecule. For example, the iCasp suicide gene comprises portion of a caspase protein operatively linked to a protein optimized to bind to an inducing agent; introduction of the inducing agent into a cell comprising the suicide gene results in the activation of caspase and the subsequent apoptosis of the cell.

The term “transduce” or “transduction” as it is applied to the production of chimeric antigen receptor cells refers to the process whereby a foreign nucleotide sequence is introduced into a cell. In some embodiments, this transduction is done via a vector.

As used herein, the term “vector” refers to a nucleic acid construct deigned for transfer between different hosts, including but not limited to a plasmid, a virus, a cosmid, a phage, a BAC, a YAC, etc. A “viral vector” is defined as a recombinantly produced virus or viral particle that comprises a polynucleotide to be delivered into a host cell, either in vivo, ex vivo or in vitro. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a gene of interest such as a polynucleotide encoding a CAR. Further details as to modern methods of vectors for use in gene transfer may be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo, and are commercially available from sources such as Agilent Technologies (Santa Clara, Calif.) and Promega Biotech (Madison, Wis.).

As used herein, the terms “T2A” and “2A peptide” are used interchangeably to refer to any 2A peptide or fragment thereof, any 2A-like peptide or fragment thereof, or an artificial peptide comprising the requisite amino acids in a relatively short peptide sequence (on the order of 20 amino acids long depending on the virus of origin) containing the consensus polypeptide motif D-V/I-E-X-N-P-G-P, wherein X refers to any amino acid generally thought to be self-cleaving.

As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, the term “signal peptide” or “signal polypeptide” intends an amino acid sequence usually present at the N-terminal end of newly synthesized secretory or membrane polypeptides or proteins. It acts to direct the polypeptide across or into a cell membrane and is then subsequently removed. Examples of such are well known in the art. Non-limiting examples are those described in U.S. Pat. Nos. 8,853,381 and 5,958,736.

As used herein in reference to a regulatory polynucleotide, the term “operatively linked” refers to an association between the regulatory polynucleotide and the polynucleotide sequence to which it is linked such that, when a specific protein binds to the regulatory polynucleotide, the linked polynucleotide is transcribed.

The term “culturing” refers to growing cells in a culture medium under conditions that favor expansion and proliferation of the cell. The term “culture medium” or “medium” is recognized in the art and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium.” “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high-density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the disclosure while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-I 2, RPMI 1640, and Iscove's Modified Dulbecco's Medium (IMDM).

“Cryoprotectants” are known in the art and include without limitation, e.g., sucrose, trehalose, and glycerol. A cryoprotectant exhibiting low toxicity in biological systems is generally used.

### Modes of Carrying Out the Disclosure

### Modified T-Cells and Methods of Producing the Same

Disclosed herein are modified T-cells modified to exhibit higher than or lower than baseline expression of one or more genes set forth in Table 1, Table 2, Table 3, Table 4, Table 5 and/or Table 7, or to express a T-cell receptor comprising, or consisting essentially of, or yet further consisting of at least one of the amino acid sequences set forth in Table 6. The one or more gene may be selected from the group of 4-1BB, PD-1, CD103 or TIM3. In one aspect, the baseline expression is normalized mean gene expression. In another aspect, the higher than baseline expression is at least about a 2-fold increase in expression relative to baseline expression and/or lower than baseline expression is at least about a 2-fold decrease in expression relative to baseline expression. Expression can be reduced or increased by at least about 2 or more, or about 3, or about 4, or about 5, or about 6, or about 7, or about 8, or about 9, or about 10, or about 11, or about 12, or about 13, or about 14, or about 15 fold as compared to a comparative wild-type cell. One of skill in the art can monitor expression of the genes using methods such as RNA-sequencing, DNA microarrays, Real-time PCR, or Chromatin immunoprecipitation (ChIP) etc. Protein expression can be monitored using methods such as flow cytometry, Western blotting, 2-D gel electrophoresis or immunoassays etc.

In a further aspect, the T-cells are tissue-resident memory cells (T_{RM}), CD8+ T-cells or tumor-infiltrating lymphocytes (TILs). In certain other aspects, the T-cells and/or TRMs are CD19−CD20−CD14−CD56−CD4−CD45+CD3+CD8 cells. In certain aspects, the T-cells and/or TRMs are TRMs expressing high levels of TIM3, CXCL13 and CD39. In one particular embodiment, the T-cells are autologous to the subject being treated.

The modified T-cell may be genetically modified, optionally using gene editing technologies, e.g., recombinant methods, CRISPR/Cas system, ZFN, and/or TALEN. Aspects of the present disclosure relate to an isolated cell comprising, or alternatively consisting essentially of, or yet further consisting of a CAR of this disclosure and methods of producing such cells. The T-cell or NK cell can be from any preferred species, e.g., an animal cell, a mammalian cell such as a human, a feline or a canine cell.

In some aspect of the present disclosure, the population of isolated cells transduced with the nucleic acid sequence encoding the CAR as described herein is a population of NK precursor cells and/or T-cell precursor cells. Transduction of precursor cells results in a long-lived population of cells capable of differentiating into CAR T-cells and/or CAR NK cells. T-cell precursors include but are not limited to HSCs; long term HSCs; MPPs; CLPs; LMPPs/ELPs; DN1s; DN2s; DN3s; DN4s; DPs. NK precursors include but are not limited to HSCs, long term HSCs, MPPs, CMPs, GMPs, pro-NK, pre-NK, and iNK cells. In a specific aspect, the population of isolated cells includes both mature T-cells and T-cell precursors to provide both short lived effector CAR T-cells and long-lived CAR T-cell precursors for transplant into the subject. In another aspect, the population of isolated cells includes both mature NK cells and NK precursors to provide both short lived effector CAR NK cells and long-lived CAR NK precursors for transplant into the subject.

In specific embodiments, the isolated cell comprises, or alternatively consists essentially of, or yet further consists of an exogenous CAR comprising, or alternatively consisting essentially of, or yet further consisting of, an antigen binding domain of the antibody provided herein, a CD8 α hinge domain, a CD8 α transmembrane domain, a CD28 costimulatory signaling region and/or a 4-1BB costimulatory signaling region, and a CD3 zeta signaling domain. In certain embodiments, the isolated cell is a T-cell, e.g., an animal T-cell, a mammalian T-cell, a feline T-cell, a canine T-cell or a human T-cell. In certain embodiments, the isolated cell is an NK-cell, e.g., an animal NK-cell, a mammalian NK-cell, a feline NK-cell, a canine NK-cell or a human NK-cell.

In some embodiments, T-cells expressing the disclosed CARs may be further modified to reduce or eliminate expression of endogenous TCRs. Reduction or elimination of endogenous TCRs can reduce off-target effects and increase the effectiveness of the T cells. T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR. Even though some TCR complexes can be recycled to the cell surface when RNA interference is used, the RNA (e.g., shRNA, siRNA, miRNA, etc.) will prevent new production of TCR proteins resulting in degradation and removal of the entire TCR complex, resulting in the production of a T cell having a stable deficiency in functional TCR expression.

Expression of inhibitory RNAs (e.g., shRNA, siRNA, miRNA, etc.) in primary T cells can be achieved using any conventional expression system, e.g., a lentiviral expression system. Although lentiviruses are useful for targeting resting primary T cells, not all T cells will express the shRNAs. Some of these T cells may not express sufficient amounts of the RNAs to allow enough inhibition of TCR expression to alter the functional activity of the T cell. Thus, T cells that retain moderate to high TCR expression after viral transduction can be removed, e.g., by cell sorting or separation techniques, so that the remaining T cells are deficient in cell surface TCR or CD3, enabling the expansion of an isolated population of T cells deficient in expression of functional TCR or CD3.

Expression of CRISPR in primary T cells can be achieved using conventional CRISPR/Cas systems and guide RNAs specific to the target TCRs. Suitable expression systems, e.g. lentiviral or adenoviral expression systems are known in the art. Similar to the delivery of inhibitor RNAs, the CRISPR system can be used to specifically target resting primary T cells or other suitable immune cells for CAR cell therapy. Further, to the extent that CRISPR editing is unsuccessful, cells can be selected for success according to the methods disclosed above. For example, as noted above, T cells that retain moderate to high TCR expression after viral transduction can be removed, e.g., by cell sorting or separation techniques, so that the remaining T cells are deficient in cell surface TCR or CD3, enabling the expansion of an isolated population of T cells deficient in expression of functional TCR or CD3. It is further appreciated that a CRISPR editing construct may be useful in both knocking out the endogenous TCR and knocking in the CAR constructs disclosed herein. Accordingly, it is appreciated that a CRISPR system can be designed for to accomplish one or both of these purposes.

Sources of Isolated Cells: Prior to expansion and genetic modification of the cells disclosed herein, cells may be obtained from a subject—for instance, in embodiments involving autologous therapy—or a commercially available culture, that are available from the American Type Culture Collection (ATCC), for example.

Cells can be obtained from a number of sources in a subject, including peripheral blood mononuclear cells, bone marrow, lymph node tissue, cord blood, thymus tissue, tissue from a site of infection, ascites, pleural effusion, spleen tissue, and tumors.

Methods of isolating relevant cells are well known in the art and can be readily adapted to the present application; an exemplary method is described in the examples below. Isolation methods for use in relation to this disclosure include but are not limited to Life Technologies Dynabeads® system; STEMcell Technologies EasySep™, RoboSep™ RosetteSep™, SepMate™; Miltenyi Biotec MACS™ cell separation kits, and other commercially available cell separation and isolation kits. Particular subpopulations of immune cells and precursors may be isolated through the use of fluorescence-activated cell sorting (FACS), beads, or other binding agents available in such kits specific to unique cell surface markers. For example, MACS™ CD4+ and CD8+ MicroBeads may be used to isolate CD4+ and CD8+ T-cells.

Alternatively, cells may be obtained through commercially available cell cultures, including but not limited to, for T-cells, lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™); and, for NK cells, lines NK-92 (ATCC® CRL-2407™), NK-92MI (ATCC® CRL-2408™).

In some aspect, the subject may be administered a conditioning regimen to induce precursor cell mobilization into the peripheral blood prior to obtaining the cells from the subject. For example, a subject may be administered an effective amount of at least one of granulocyte colony-stimulating factor (G-CSF), filgrastim (Neupogen), sargramostim (Leukine), pegfilgrastim (Neulasta), and mozobil (Plerixafor) up to two weeks prior to or concurrently with isolation of cells from the subject. Mobilized precursor cells can be obtained from the subject by any method known in the art, including, for example, leukapheresis 1-14 days following administration of the conditioning regimen.

Activation and Expansion of T Cells: Whether prior to or after genetic modification of the T cells to express a desirable CAR, the cells can be activated and expanded using generally known methods such as those described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041. Methods of activating relevant cells are well known in the art and can be readily adapted to the present application; an exemplary method is described in the examples below. Isolation methods for use in relation to this disclosure include but are not limited to Life Technologies Dynabeads® system activation and expansion kits; BD Biosciences Phosflow™ activation kits, Miltenyi Biotec MACS™ activation/expansion kits, and other commercially available cell kits specific to activation moieties of the relevant cell. Particular subpopulations of immune cells may be activated or expanded through the use of beads or other agents available in such kits. For example, α-CD3/α-CD28 Dynabeads® may be used to activate and expand a population of isolated T-cells.

Also disclosed herein is an isolated cell comprising, or alternatively consisting essentially of, or yet further consisting of the CAR of this disclosure.

The modified T-cell disclosed herein can also be further modified to express a protein that binds to a cytokine, chemokine, lymphokine, or a receptor each thereof. In one aspect, the protein comprises, or consists essentially of, or yet further consists of an antibody or an antigen binding fragment thereof.

In another aspect, the antibody is an IgG, IgA, IgM, IgE or IgD, or a subclass thereof. The antibody can also be an IgG selected from the group of IgG1, IgG2, IgG3 or IgG4. Furthermore, the antigen binding fragment can be selected from the group of a Fab, Fab′, F(ab′)2, Fv, Fd, single-chain Fvs (scFv), disulfide-linked Fvs (sdFv) or VL or VH.

In one aspect, the modified T-cell of this disclosure comprises, or consists essentially of, or yet further consists of a chimeric antigen receptor (CAR). In one embodiment, the chimeric antigen receptor (CAR) comprises, or consists essentially of, or yet further consists of: (a) an antigen binding domain; (b) a hinge domain; (c) a transmembrane domain; (d) and an intracellular domain.

Spacer Domain: The CARs may optionally further comprise, or alternatively consist essentially of, or yet further consist of a spacer domain of up to 300 amino acids, preferably 10 to 100 amino acids, more preferably 25 to 50 amino acids. For example, the spacer may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. A spacer domain may comprise, for example, a portion of a human Fc domain, a CH₃ domain, or the hinge region of any immunoglobulin, such as IgA, IgD, IgE, IgG, or IgM, or variants thereof. For example, some embodiments may comprise an IgG₄ hinge with or without a S228P, L235E, and/or N297Q mutation (according to Kabat numbering). Additional spacers include, but are not limited to, CD4, CD8, and CD28 hinge regions.

Transmembrane Domain. The transmembrane domain may be derived either from a natural or from a synthetic source. Where the source is natural, the domain may be derived from any membrane-bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Cytoplasmic Domain. The cytoplasmic domain or intracellular signaling domain of the CAR is responsible for activation of at least one of the traditional effector functions of an immune cell in which a CAR has been placed. The intracellular signaling domain refers to a portion of a protein which transduces the effector function signal and directs the immune cell to perform its specific function. An entire signaling domain or a truncated portion thereof may be used so long as the truncated portion is sufficient to transduce the effector function signal. Cytoplasmic sequences of the T-cell receptor (TCR) and co-receptors, as well as derivatives or variants thereof, can function as intracellular signaling domains for use in a CAR. Intracellular signaling domains of particular use in this disclosure may be derived from FcR, TCR, CD3, CDS, CD22, CD79a, CD79b, CD66d. In some embodiments, the signaling domain of the CAR comprises, or consists essentially thereof, or consists of a CD3 ζ signaling domain.

Co-stimulatory Domains. Since signals generated through the TCR are alone insufficient for full activation of a T cell, a secondary or co-stimulatory signal may also be required. Thus, the intracellular region of at least one co-stimulatory signaling molecule, including but not limited to CD27, CD28, 4-1BB (CD 137), OX40, CD30, CD40, PD-1, ICOS, lymphocyte function-associated antigen-1 (LFA-1), CD2, CD7, LIGHT, NKG2C, B7-H3, or a ligand that specifically binds with CD83, may also be included in the cytoplasmic domain of the CAR. CARs of the present disclosure can comprise, or consist essentially thereof, or consist of one or more co-stimulatory domain. For instance, a CAR may comprise, or consist essentially thereof, or consist of one, two, or more co-stimulatory domains, in addition to a signaling domain (e.g., a CD3 signaling domain).

In some embodiments, the cell activation moiety of the chimeric antigen receptor is a T-cell signaling domain comprising, or alternatively consisting essentially of, or yet further consisting of, one or more proteins or fragments thereof selected from the group consisting of CD8 protein, CD28 protein, 4-1BB protein, OX40, CD30, CD40, PD-1, ICOS, LFA-1, CD2, CD7, CD27, LIGHT, NKG2C, B7-H3 and CD3-zeta protein.

In specific embodiments, the CAR comprises, or alternatively consists essentially thereof, or yet consists of an antigen binding domain of an any of the antibodies of this disclosure or fragment (e.g., scFv) thereof, a CD8 α or an IgG1 hinge domain, a CD8 α transmembrane domain, at least one costimulatory signaling region, and a CD3 zeta signaling domain. In further embodiments, the costimulatory signaling region comprises, or alternatively consists essentially thereof, or yet consists of either or both a CD28 costimulatory signaling region and a 4-1BB costimulatory signaling region.

In one embodiment, the antigen binding domain comprises, or consists essentially of, or yet further consists of an anti-CD19 antigen binding domain, the transmembrane domain comprises, or consists essentially of, or yet further consists of a CD28, CD28H (TMIGD2), AMICA1 or a CD8 α transmembrane domain and the one or more costimulatory regions selected from a CD28 costimulatory signaling region, a 4-1BB costimulatory signaling region, an ICOS costimulatory signaling region, an AMICA1 costimulatory signaling region, a CD28H (TMIGD2) costimulatory signaling region, and an OX40 costimulatory region or a CD3 zeta signaling domain. In a further embodiment, the anti-CD19 binding domain comprises, or consists essentially of, or yet further consists of a single-chain variable fragment (scFv) that specifically recognizes a humanized anti-CD19 binding domain. The anti-CD19 binding domain scFv of the CAR may comprise, or consist essentially of, or yet further consist of a heavy chain variable region and a light chain variable region.

In one aspect, the anti-CD19 binding domain of the CAR further comprises, or consists essentially of, or yet further consists of a linker polypeptide located between the anti-CD19 binding domain scFv heavy chain variable region and the anti-CD19 binding domain scFv light chain variable region. The linker polypeptide of the CAR may comprise, or consist essentially of, or yet further consist of a polypeptide of the sequence (GGGGS)n wherein n is an integer from 1 to 6. The linker peptide may be from 1 to 50 amino acids, for instance, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. In some embodiments, the linker is glycine rich, although it may also contain serine or threonine. In another aspect, the CAR can further comprise, or consist essentially of, or yet further consist of a detectable marker attached to the CAR. In a separate aspect, the CAR can further comprise, or consist essentially of, or yet further consist of a purification marker attached to the CAR.

Switch Mechanisms. In some embodiments, the CAR may also comprise, or consist essentially thereof, or consist of a switch mechanism for controlling expression and/or activation of the CAR. For example, a CAR may comprise, consist, or consist essentially of an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises, consists, or consists essentially of a target antigen binding domain and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but it cannot bind its target antigen until the second composition comprising a specific binding domain is administered.

CARs of the present disclosure may likewise require multimerization in order to activate their signaling function (see, e.g., US 2015/0368342, US 2016/0175359, US 2015/0368360) and/or an exogenous signal, such as a small molecule drug (US 2016/0166613, Yung et al., Science, 2015) in order to elicit a T-cell response.

Furthermore, the disclosed CARs can comprise, or consist essentially thereof, or consist of a “suicide switch” to induce cell death of the CAR T-cells following treatment (Buddee et al., PLoS One, 2013) or to downregulate expression of the CAR following binding to the target antigen (WO 2016/011210).

Also provided herein are modified T-cells prepared by any of the methods disclosed below. Further provided herein is a substantially homogenous population of cells of any of the modified T-cells of this disclosure. Also provided herein is a heterogeneous population of cells of any of the modified T-cells of this disclosure.

In one aspect, the method of producing the modified T-cells comprises, or alternatively consists essentially of, or yet further consists of isolating the T-cells and culturing the cells under conditions that favor expansion and proliferation of the cells. The modified T-cell may be genetically modified, optionally using recombinant methods, CRISPR/Cas system, ZFN, and/or TALEN.

CARs may be prepared using vectors. Aspects of the present disclosure relate to an isolated nucleic acid sequence encoding the CARs disclosed herein and vectors comprising, or alternatively consisting essentially of, or yet further consisting of an isolated nucleic acid sequence encoding the CAR and its complement and equivalents of each thereof.

The CAR cells of this disclosure can be generated by inserting into the modified T-cell a polynucleotide encoding the CAR and then expressing the CAR in the cell, Thus, in one aspect, the engineered T cell of this disclosure comprises, or alternatively consists essentially of, or yet further consists of a polynucleotide encoding the CAR, wherein the polynucleotide further comprises, or alternatively consists essentially of, or yet further consists of a promoter operatively linked to the polynucleotide to express the polynucleotide in the cell. Non-limiting examples of promoters include constitutive, inducible, repressible, or tissue-specific. The promoter is “operatively linked” in a manner to transcribe the linked polynucleotide.

Further provided herein is a modified T-cell comprising, or consisting essentially of, or yet further consisting of a polynucleotide encoding the CAR, and optionally, wherein the polynucleotide encodes and anti-CD19 binding domain. In one aspect, the polynucleotide may further comprise, or consist essentially of, or yet further consist of a promoter operatively linked to the polynucleotide to express the polynucleotide in the modified T-cell. In another aspect, the polynucleotide may further comprise, or consist essentially of, or yet further consist of a 2A self-cleaving peptide (T2A) encoding polynucleotide sequence located upstream of a polynucleotide encoding the anti-CD19 binding domain. “T2A” and “2A peptide” are used interchangeably to refer to any 2A peptide or fragment thereof, any 2A-like peptide or fragment thereof, or an artificial peptide comprising the requisite amino acids in a relatively short peptide sequence (on the order of 20 amino acids long depending on the virus of origin) containing the consensus polypeptide motif D-V/I-E-X-N-P-G-P, wherein X refers to any amino acid generally thought to be self-cleaving.

In yet a further aspect, the polynucleotide may further comprise, or consist essentially of, or yet further consist of a polynucleotide encoding a signal peptide located upstream of a polynucleotide encoding the anti-CD19 binding domain. In some embodiments, the polynucleotide comprises, or alternatively consists essentially thereof, or yet further consists of, a Kozak consensus sequence upstream of the polynucleotide sequence encoding the antigen binding domain or an enhancer. In some embodiments, the polynucleotide comprises, or alternatively consists essentially thereof, or yet further consists of a polynucleotide conferring antibiotic resistance. In one particular embodiment, the isolated nucleic acid encoding the CAR further comprises, or alternatively consists essentially thereof, or yet further consists of a switch mechanism for controlling expression and/or activation of the CAR.

The preparation of exemplary vectors and the generation of CAR expressing cells using the vectors is discussed in detail in the examples below. In summary, the expression of natural or synthetic nucleic acids encoding CARs is typically achieved by operably linking a nucleic acid encoding the CAR polypeptide or portions thereof to a promoter and incorporating the construct into an expression vector. The vectors can be suitable for replication and integration eukaryotes. Methods for producing cells comprising vectors and/or exogenous nucleic acids are well-known in the art. See, for example, Sambrook et al. (2001, Molecular Cloning A Laboratory Manual, Cold Spring Harbor Laboratory, New York).

In several aspects, the vector is derived from or based on a wild-type virus. In further aspects, the vector is derived from or based on a wild-type lentivirus. Examples of such, include without limitation, human immunodeficiency virus (HIV), equine infectious anemia virus (EIAV), simian immunodeficiency virus (SW) and feline immunodeficiency virus (FIV). Alternatively, it is contemplated that other retrovirus can be used as a basis for a vector backbone such murine leukemia virus (MLV). It will be evident that a viral vector according to the disclosure need not be confined to the components of a particular virus. The viral vector may comprise components derived from two or more different viruses and may also comprise synthetic components. Vector components can be manipulated to obtain desired characteristics, such as target cell specificity.

The recombinant vectors of this disclosure may be derived from primates and non-primates. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anemia virus (EIAV) and the more recently described feline immunodeficiency virus (FIV) and bovine immunodeficiency virus (BIV). Prior art recombinant lentiviral vectors are known in the art, e.g., see U.S. Pat. Nos. 6,924,123; 7,056,699; 7,07,993; 7,419,829 and 7,442,551, incorporated herein by reference.

U.S. Pat. No. 6,924,123 discloses that certain retroviral sequence facilitate integration into the target cell genome. This patent teaches that each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′end of the RNA. The sizes of the three elements can vary considerably among different retroviruses. For the viral genome. and the site of poly (A) addition (termination) is at the boundary between R and U5 in the right-hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

For the production of viral vector particles, the vector RNA genome is expressed from a DNA construct encoding it, in a host cell. The components of the particles not encoded by the vector genome are provided in trans by additional nucleic acid sequences (the “packaging system”, which usually includes either or both of the gag/pol and env genes) expressed in the host cell. The set of sequences required for the production of the viral vector particles may be introduced into the host cell by transient transfection, or they may be integrated into the host cell genome, or they may be provided in a mixture of ways. The techniques involved are known to those skilled in the art.

Retroviral vectors for use in this disclosure include but are not limited to Invitrogen's pLenti series versions 4, 6, and 6.2 “ViraPower” system. Manufactured by Lentigen Corp.; pHIV-7-GFP, lab generated and used by the City of Hope Research Institute; “Lenti-X” lentiviral vector, pLVX, manufactured by Clontech; pLKO.1-puro, manufactured by Sigma-Aldrich; pLemiR, manufactured by Open Biosystems; and pLV, lab generated and used by Charité Medical School, Institute of Virology (CBF), Berlin, Germany.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

Packaging vector and cell lines: CARs can be packaged into a lentiviral or retroviral packaging system by using a packaging vector and cell lines. The packaging plasmid includes, but is not limited to retroviral vector, lentiviral vector, adenoviral vector, and adeno-associated viral vector. The packaging vector contains elements and sequences that facilitate the delivery of genetic materials into cells. For example, the retroviral constructs are packaging plasmids comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer and/or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer and/or promoter which directs efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter. The retroviral packaging plasmid may consist of two retroviral helper DNA sequences encoded by plasmid-based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the aforementioned env genes or chimeric envelope genes encoding the cytoplasmic and transmembrane of the aforementioned env gene products and a monoclonal antibody directed against a specific surface molecule on a desired target cell.

In the packaging process, the packaging plasmids and retroviral vectors are transiently co-transfected into a first population of mammalian cells that are capable of producing virus, such as human embryonic kidney cells, for example 293 cells (ATCC No. CRL1573, ATCC, Rockville, Md.), to produce high titer recombinant retrovirus-containing supernatants. In another method of the disclosure this transiently transfected first population of cells is then co-cultivated with mammalian target cells, for example human lymphocytes, to transduce the target cells with the foreign gene at high efficiencies. In yet another method of the disclosure the supernatants from the above described transiently transfected first population of cells are incubated with mammalian target cells, for example human lymphocytes or hematopoietic stem cells, to transduce the target cells with the foreign gene at high efficiencies.

In another aspect, the packaging plasmids are stably expressed in a first population of mammalian cells that are capable of producing virus, such as human embryonic kidney cells, for example 293 cells. Retroviral or lentiviral vectors are introduced into cells by either co-transfection with a selectable marker or infection with pseudotyped virus. In both cases, the vectors integrate. Alternatively, vectors can be introduced in an episomally maintained plasmid. High titer recombinant retrovirus-containing supernatants are produced.

In one embodiment, the polynucleotide further comprises, or consists essentially of, or yet further consists of a vector. In one particular embodiment, the vector is a plasmid. In another embodiment, the vector is a viral vector selected from the group of a retroviral vector, a lentiviral vector, an adenoviral vector, and an adeno-associated viral vector.

In some embodiments, the T cell of this disclosure has been isolated from a subject. In a particular embodiment, the T cell of this disclosure has been isolated from a subject, wherein the subject has cancer. In one aspect, the cancer or tumor is an epithelial, a head, neck, lung, lung, prostate, colon, pancreas, esophagus, liver, skin, kidney, adrenal gland, brain, or comprises a lymphoma, breast, endometrium, uterus, ovary, testes, lung, prostate, colon, pancreas, esophagus, liver, skin, kidney, adrenal gland and/or brain cancer or tumor, a metastasis or recurring tumor, cancer or neoplasia, a non-small cell lung cancer (NSCLC) and/or head and neck squamous cell cancer (HNSCC). In another aspect the subject is The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

### Compositions, Methods of Treatment, Diagnosis and Prognosis

Also disclosed herein is a composition comprising, or consisting essentially of, or yet further consisting of a population of modified T-cells described above. Further provided herein is a composition comprising, or alternatively consisting essentially of, or yet further consisting of a carrier and one or more of: the modified T cell of this disclosure and/or the population of modified T-cells of this disclosure. In one aspect, the population is a substantially homogenous cell population. In another aspect, the population is a heterogeneous population. The composition of the present disclosure also can be bound to many different carriers. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the disclosure. Those skilled in the art will know of other suitable carriers, or will be able to ascertain such, using routine experimentation.

Further provided herein are methods to identify the antigens or antigen receptors associated with the isolated and/or purified cell populations disclosed herein. In some aspect, the receptors are T-cell receptors (TCRs). In particular embodiments, the TCRs comprise the sequences listed in Table 6. In certain embodiments, the identified antigens or antigen receptors can be used for example to vaccinate a subject against cancer or an immune response. In other aspects, the identified antigens or antigen receptors can be used to engineer cells, for example a chimeric-antigen receptor T-cell (CAR-T cell). In still other aspects, the engineered CAR-T cell can be used to provide immunotherapy to a subject such as for example, a human patient. Also provided herein are methods to induce an immune response and treat conditions requiring selective immunotherapy, comprising, or consisting essentially of, or yet further consisting of, contacting a target cell with the cells or compositions as described herein. The contacting can be performed in vitro, or alternatively in vivo, thereby providing immunotherapy to a subject such as for example, a human patient.

Provided herein are methods to identify the antigens or antigen receptors associated with the isolated and/or purified cell populations disclosed herein. In some aspect, the receptors are T-cell receptors (TCRs). In particular embodiments, the TCRs comprise the sequences listed in Table 6. In certain embodiments, the identified antigens or antigen receptors can be used for example to vaccinate a subject against cancer or an immune response. In other aspects, the identified antigens or antigen receptors can be used to engineer cells, for example a chimeric-antigen receptor T-cell (CAR-T cell). In still other aspects, the engineered CAR-T cell can be used to provide immunotherapy to a subject such as for example, a human patient.

Also provided herein are methods to induce an immune response and treat conditions requiring selective immunotherapy, comprising, or consisting essentially of, or yet further consisting of, contacting a target cell with the cells or compositions as described herein.

Provided herein is a method of treating cancer, providing anti-tumor immunity, preventing relapse of cancer, and/or eliciting an anti-tumor response in a subject comprising, or consisting essentially of, or yet further consisting of administering to the subject an effective amount of a population of T-cells that exhibit higher than or lower than baseline expression of one or more genes set forth in Table 1, Table 2, Table 3, Table 4, Table 5 and/or Table 7, or that express a T-cell receptor comprising at least one of the amino acid sequences set forth in Table 6. Providing anti-tumor immunity refers to preventing the symptoms or cancer from occurring in a subject that is predisposed or does not yet display symptoms of the cancer. In another aspect, it is to inhibit relapse or progression of cancer in a subject in need thereof.

In one aspect, the method comprises, or consists essentially of, or yet further consists of administering to the subject an effective amount of an agent that induces higher than or lower than baseline expression of one or more genes set forth in Table 1, Table 2, Table 3, Table 4, Table 5 and/or Table 7 in T-cells, or a T-cell receptor comprising at least one of the amino acid sequences set forth in Table 6. In another aspect, the method comprises, or consists essentially of, or yet further consists of administering an effective amount of one or more an agent that induces or inhibits in T-cells activity of one or more proteins encoded by genes set forth in Table 1, Table 2, Table 3, Table 4, Table 5 and/or Table 7 to the subject or sample. The active agent can be an antibody, a small molecule, a protein, a peptide, a ligand mimetic or a nucleic acid. The one or more gene may be selected from the group of 4-1BB, PD-1, CD103 or TIM3. In one aspect, the baseline expression is normalized mean gene expression. In another aspect, the higher than baseline expression is at least about a 2-fold increase in expression relative to baseline expression and/or lower than baseline expression is at least about a 2-fold decrease in expression relative to baseline expression. Expression can be reduced or increased by at least about 2 or more, or about 3, or about 4, or about 5, or about 6, or about 7, or about 8, or about 9, or about 10, or about 11, or about 12, or about 13, or about 14, or about 15 fold as compared to a comparative wild-type cell. One of skill in the art can monitor expression of the genes using methods such as RNA-sequencing, DNA microarrays, Real-time PCR, or Chromatin immunoprecipitation (ChIP) etc. Protein expression can be monitored using methods such as flow cytometry, Western blotting, 2-D gel electrophoresis or immunoassays etc.

In a further aspect, the T-cells are tissue-resident memory cells (TRM) or CD8+ T-cells. In one particular embodiment, the T-cells are autologous to the subject being treated. The methods of treating cancer, providing anti-tumor immunity, preventing relapse of cancer, and/or eliciting an anti-tumor response disclosed herein may further comprise, or consist essentially of, or yet further consist of administering to the subject an effective amount of a cytoreductive therapy. The cytoreductive therapy can be one or more of chemotherapy, immunotherapy, or radiation therapy.

Further provided herein is a method of treating cancer in a subject and/or eliciting an anti-tumor response comprising, or consisting essentially of, or yet further consisting of administering to the subject or contacting the tumor with an effective amount of the modified T-cells disclosed herein and/or the composition of this disclosure. The contacting can be performed in vitro, or alternatively in vivo, thereby providing immunotherapy to a subject such as for example, a human patient. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

In one aspect, for the methods of treatments, the subject has, has had or is in need of treatment for cancer. In another aspect, the cancer is characterized as being hyporesponsive. In certain embodiments a subject has or is suspected of having a neoplastic disorder, neoplasia, tumor, malignancy or cancer. In some embodiments a subject in need of a treatment, cell or composition described herein has or is suspected of having a neoplastic disorder, neoplasia, tumor, malignancy or cancer.

The T-cells, population of T-cells, active agent and/or compositions provided herein may be administered either alone or in combination with diluents, known anti-cancer therapeutics, and/or with other components such as cytokines or other cell populations that are immunostimulatory. They may be administered as a first line therapy, a second line therapy, a third line therapy, or further therapy. Non-limiting examples of additional therapies include chemotherapeutics or biologics. Appropriate treatment regimens will be determined by the treating physician or veterinarian.

In one embodiment, the tumor is a solid tumor. The solid tumor could be a melanoma, a colon carcinoma, a breast carcinoma and/or a brain tumor. In one aspect, the cancer to be treated is a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, or melanoma.

The methods are useful to treat subjects such as humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). A mammal can be any age or at any stage of development (e.g., an adult, teen, child, infant, or a mammal in utero). A mammal can be male or female. In certain embodiments the subject has or is suspected of having a neoplastic disorder, neoplasia, tumor, malignancy or cancer. In one aspect, the animal is treated as an animal model for a particular patient or tumor type, or can be used to assay combination therapies.

The methods disclosed herein may further comprise or alternatively consist essentially of, or yet further consists of administering to the subject an anti-tumor therapy other than the CAR therapy or T-cell therapy as disclosed herein. Accordingly, method aspects of the present disclosure relate to methods for inhibiting the growth of a tumor in a subject in need thereof and/or for treating a cancer patient in need thereof.

Further provided herein is a method of diagnosing a subject that may optionally be suspected of having cancer, comprising, or consisting essentially of, or yet further consisting of contacting a sample isolated from the subject with an agent that detects the presence of one or more genes set forth in Table 1, Table 2, Table 3, Table 4, Table 5 and/or Table 7 in the sample isolated from the subject, wherein the presence of the one or more genes at higher or lower than baseline expression levels is diagnostic of cancer. In one aspect, the method of diagnosing cancer in a subject comprises, or consists essentially of, or yet further consists of contacting tissue-resident memory cells (TRMs) of the cancer or a sample thereof with an antibody or agent that recognizes and binds CD8, an antibody or agent that recognizes and binds PD-1, an antibody or agent that recognizes and binds TIM3, an antibody or agent that recognizes and binds LAG3, an antibody or agent that recognizes and binds AMICA1, an antibody or agent that recognizes and binds CD28H (TMIGD2), and an antibody or agent that recognizes and binds CTLA4 to determine the frequency of CD8^{+PD}1⁺, CD8⁺TIM3⁺, CD8⁺LAG3⁺, CD8⁺AMICA1⁺, CD8⁺CD28H⁺, CD8⁺CTLA4⁺, CD8⁺PD1⁺TIM3⁺, CD8⁺PD1⁺LAG3⁺, CD8⁺PD1⁺AMICA1⁺, CD8⁺PD1⁺CD28H⁺, CD8⁺PD1⁺CTLA4⁺, CD8⁺TIM3⁺LAG3⁺, CD8⁺TIM3⁺AMICA1⁺, CD8⁺TIM3⁺CD28H⁺, CD8⁺TIM3⁺CTLA4⁺, CD8⁺LAG3⁺CTLA4⁺, CD8⁺LAG3⁺AMICA1⁺, CD8⁺LAG3⁺CD28H⁺, CD8⁺PD1⁺TIM3⁺LAG3⁺, CD8⁺LAG3⁺PD1⁺AMICA1⁺, CD8⁺LAG3⁺PD1⁺CD28H⁺, CD8⁺PD1⁺LAG3⁺CTLA4⁺, CD8⁺PD1⁺TIM3⁺CTLA4⁺, CD8⁺PD1⁺TIM3⁺CTLA4⁺AMICA1⁺′, CD8⁺PD1⁺TIM3⁺CTLA4⁺CD28H⁺′ or CD8⁺PD1⁺TIM3⁺CTLA4⁺AMICA⁺CD28H⁺′ TRMs, wherein a high frequency of one or more of these TRMs is diagnostic of cancer.

In another aspect, the method of diagnosing cancer in a subject comprises, or consists essentially of, or yet further consists of contacting tissue-resident memory cells (TRMs) isolated from the subject or cancer sample isolated from the subject, with an antibody or agent that recognizes and binds one or more proteins encoded by a gene set forth in Table 1, Table 2, Table 3, Table 4, Table 5 and/or Table 7 and, optionally, an antibody or agent that recognizes and binds CD8, an antibody or agent that recognizes and binds PD-1, an antibody or agent that recognizes and binds TIM3, an antibody or agent that recognizes and binds LAG3, an antibody or agent that recognizes and binds CD28H (TMIGD2), an antibody or agent that recognizes and binds AMICA1, an antibody or agent that recognizes and binds KLF3, an antibody or agent that recognizes and binds S1PR5, an antibody or agent that recognizes and binds S1PR1, an antibody or agent that recognizes and binds KLF2 and an antibody or agent that recognizes and binds CTLA4 to determine the frequency of TRMs expressing these proteins, wherein a high frequency of TRMs expressing these proteins is diagnostic of cancer. The contacting can be performed in vitro, or alternatively in vivo. The subject can be any mammal, e.g., a human patient. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration. Expression can be reduced or increased by at least about 2 or more, or about 3, or about 4, or about 5, or about 6, or about 7, or about 8, or about 9, or about 10, or about 11, or about 12, or about 13, or about 14, or about 15 fold as compared to a comparative wild-type cell. One of skill in the art can monitor expression of the genes using methods such as RNA-sequencing, DNA microarrays, Real-time PCR, or Chromatin immunoprecipitation (ChIP) etc. Protein expression can be monitored using methods such as flow cytometry, Western blotting, 2-D gel electrophoresis or immunoassays etc.

Additionally, disclosed herein is a method of determining the density of tissue-resident memory cells (TRMs) in a subject or sample isolated from the subject, e.g., a cancer, tumor, or sample thereof, the method comprising, or consisting essentially of, or yet further consisting of measuring expression of one or more gene selected from the group of 4-1BB, PD-1, CD103 or TIM3 or genes set forth in Table 1, Table 2, Table 3, Table 4, Table 5 and/or Table 7 in the sample, (e.g., cancer, tumor, or sample thereof), wherein higher or lower than baseline expression indicates higher density of TRMs in the sample (e.g., cancer, tumor, or sample thereof). Expression can be reduced or increased by at least about 2 or more, or about 3, or about 4, or about 5, or about 6, or about 7, or about 8, or about 9, or about 10, or about 11, or about 12, or about 13, or about 14, or about 15 fold as compared to a comparative wild-type cell. One of skill in the art can monitor expression of the genes using methods such as RNA-sequencing, DNA microarrays, Real-time PCR, or Chromatin immunoprecipitation (ChIP) etc. Protein expression can be monitored using methods such as flow cytometry, Western blotting, 2-D gel electrophoresis or immunoassays etc.

Further provided herein is a method of determining prognosis of a subject having cancer comprising, or consisting essentially of, or yet further consisting of measuring the density of tissue-resident memory cells (T_{RM}) in a sample isolated from the subject, (e.g., the cancer, tumor or a sample thereof), wherein a high density of T_{RM} indicates a more positive prognosis, e.g., an increased probability and/or duration of survival. In one aspect, the method of prognosis of a subject having cancer comprises, or consists essentially of, or yet further consists of contacting tissue-resident memory cells (TRMs) isolated from the subject with an antibody or agent that recognizes and binds CD8, an antibody or agent that recognizes and binds PD-1, an antibody or agent that recognizes and binds TIM3, an antibody or agent that recognizes and binds LAG3, an antibody or agent that recognizes and binds AMICA1, an antibody or agent that recognizes and binds CD28H (TMIGD2), and an antibody or agent that recognizes and binds CTLA4 to determine the frequency of CD8⁺PD1⁺, CD8⁺TIM3⁺, CD8⁺LAG3⁺, CD8⁺AMICA1⁺, CD8⁺CD28H⁺, CD8⁺CTLA4⁺, CD8⁺PD1⁺TIM3⁺, CD8⁺PD1⁺LAG3⁺, CD8⁺PD1⁺AMICA1⁺, CD8⁺PD1⁺CD28H⁺, CD8⁺PD1⁺CTLA4⁺, CD8⁺TIM3⁺LAG3⁺, CD8⁺TIM3⁺AMICA1⁺, CD8⁺TIM3⁺CD28H⁺, CD8⁺TIM3⁺CTLA4⁺, CD8⁺LAG3⁺CTLA4⁺, CD8⁺LAG3⁺AMICA1⁺, CD8⁺LAG3⁻⁺CD28H⁺, CD8⁺PD1⁺TIM3⁺LAG3⁺, CD8⁺LAG3⁺PD1⁺AMICA1⁺, CD8⁺LAG3⁺PD1⁺CD28H⁺, CD8^{+PD}1⁺LAG3⁺CTLA4⁺, CD8⁺PD1⁺TIM3⁺CTLA4⁺, CD8⁺PD1⁺TIM3⁺CTLA4⁺AMICA1⁺′, CD8⁺PD1^{.+}TIM3⁺CTLA4⁺CD28H⁺′ or CD8⁺PD1⁺TIM3⁺CTLA4⁺AMICA⁺CD28H⁺′ TRMs, wherein a high frequency of one or more of these TRMs indicates a more positive prognosis, e.g., an increased probability and/or duration of survival. In another aspect, the method of prognosis of a subject having cancer comprises, or consists essentially of, or yet further consists of contacting tissue-resident memory cells (TRMs) isolated from the subject, (e.g., of the cancer or a sample thereof) with an antibody or agent that recognizes and binds one or more proteins encoded by a gene set forth in Table 1, Table 2, Table 3, Table 4, Table 5 and/or Table 7 and, optionally, an antibody or agent that recognizes and binds CD8, an antibody or agent that recognizes and binds PD-1, an antibody or agent that recognizes and binds TIM3, an antibody or agent that recognizes and binds LAG3, an antibody or agent that recognizes and binds CD28H (TMIGD2), an antibody or agent that recognizes and binds AMICA1, an antibody or agent that recognizes and binds KLF3, an antibody or agent that recognizes and binds S1PR5, an antibody or agent that recognizes and binds S1PR1, an antibody or agent that recognizes and binds KLF2 and an antibody or agent that recognizes and binds CTLA4 to determine the frequency of TRMs expressing these proteins, wherein a high frequency of TRMs expressing these proteins indicates a more positive prognosis, e.g., an increased probability and/or duration of survival.

In yet a further aspect, the method of determining prognosis of a subject having cancer comprises, or consists essentially of, or yet further consists of contacting tissue-resident memory cells (TRMs) isolated from the subject, e.g., of the cancer or a sample thereof; with an antibody or agent that recognizes and binds CD103 to determine the frequency of CD103+ TRMs or an antibody that recognizes and binds a protein encoded by a gene set forth in Table 1, Table 2, Table 3, Table 4, Table 5 and/or Table 7 to determine the frequency of TRMs expressing the protein, wherein a high or low frequency of TRMs expressing the protein indicates a more positive prognosis, e.g., an increased probability and/or duration of survival. The contacting can be performed in vitro, or alternatively in vivo. The subject can be a mammal, e.g., a human patient. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration. In a separate aspect, the method of determining prognosis of a subject having cancer comprises, or consists essentially of, or yet further consists of measuring the density of CD103 or proteins encoded by one or more gene set forth in Table 1, Table 2, Table 3, Table 4, Table 5 and/or Table 7 in the sample, (e.g., a cancer or a sample thereof), wherein a high or low density of proteins indicates a more positive prognosis, and an increased probability and/or duration of survival.

For the above methods, an effective amount is administered, and administration of the cell or population serves to attenuate any symptom or prevent additional symptoms from arising. When administration is for the purposes of preventing, delaying or reducing the likelihood of cancer recurrence or metastasis or pathogen infection, the cell or compositions can be administered in advance of any visible or detectable symptom. Routes of administration include, but are not limited to, oral (such as a tablet, capsule or suspension), topical, transdermal, intranasal, vaginal, rectal, subcutaneous intravenous, intraarterial, intramuscular, intraosseous, intraperitoneal, epidural and intrathecal. In some embodiments, an effective amount may be delivered or administered into a cavity formed by the resection of tumor tissue (i.e. intracavity delivery) or directly into a tumor prior to resection (i.e. intratumoral delivery). In some embodiments, administration can be intravenously, intrathecally, intraperitoneally, intramuscularly, subcutaneously, or by other suitable means of administration.

Pharmaceutical compositions of the present disclosure may be administered in a manner appropriate to the disease to be treated or prevented. The quantity and frequency of administration will be determined by such factors as the condition of the patient, and the type and severity of the patient's disease, although appropriate dosages may be determined by clinical trials.

For the above methods, an effective amount is administered, and administration of the cell or population serves to attenuate any symptom or prevent additional symptoms from arising. When administration is for the purposes of preventing or reducing the likelihood of cancer recurrence or metastasis, the cell or compositions can be administered in advance of any visible or detectable symptom. Routes of administration include, but are not limited to, oral (such as a tablet, capsule or suspension), topical, transdermal, intranasal, vaginal, rectal, subcutaneous intravenous, intraarterial, intramuscular, intraosseous, intraperitoneal, epidural and intrathecal.

The methods provide one or more of: (1) preventing the symptoms or disease from occurring in a subject that is predisposed or does not yet display symptoms of the disease; (2) inhibiting the disease or arresting its development; or (3) ameliorating or causing regression or relapse of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies e.g., surgical recession, chemotherapy, radiation. In one aspect, treatment excludes prophylaxis.

Also described herein is a method of determining the responsiveness of a subject having cancer to immunotherapy comprising, or consisting essentially of, or yet further consisting of contacting tissue-resident memory cells (TRMs) isolated from the subject, e.g., of the cancer or a sample thereof, with an antibody or agent that recognizes and binds CD8, an antibody or agent that recognizes and binds PD-1, an antibody or agent that recognizes and binds TIM3, an antibody or agent that recognizes and binds LAG3, an antibody or agent that recognizes and binds AMICA1, an antibody or agent that recognizes and binds CD28H (TMIGD2), and an antibody or agent that recognizes and binds CTLA4 to determine the frequency of CD8⁺PD1⁺, CD8⁺TIM3⁺, CD8⁺LAG3⁺, CD8⁺AMICA1⁺, CD8⁺CD28H⁺, CD8⁺CTLA4⁺, CD8⁺PD1⁺TIM3⁺, CD8⁺PD1⁺LAG3⁺, CD8⁺PD1⁺AMICA1⁺, CD8⁺PD1⁺CD28H⁺, CD8⁺PD1⁺CTLA4⁺, CD8⁺TIM3⁺LAG3⁺, CD8⁺TIM3⁺AMICA1⁺, CD8⁺TIM3⁺CD28H⁺, CD8⁺TIM3⁺CTLA4⁺, CD8⁺LAG3⁺CTLA4⁺, CD8⁺LAG3⁺AMICA1⁺, CD8⁺LAG3⁺CD28H⁺, CD8^{+PD}1⁺TIM3⁺LAG3⁺, CD8⁺LAG3⁺PD1⁺AMICA1⁺, CD8⁺LAG3⁺PD1⁺CD28H⁺, CD8⁺PD1⁺LAG3⁺CTLA4⁺, CD8⁺PD1⁺TIM3⁺CTLA4⁺, CD8⁺PD1⁺TIM3⁺CTLA4⁺AMICA1⁺′, CD8⁺PD1⁺TIM3⁺CTLA4⁺CD28H⁺′ or CD8⁺PD1⁺TIM3⁻⁺CTLA4⁺AMICA⁺CD28H⁺′TRMs, wherein a high frequency of one or more of these TRMs indicates responsiveness to immunotherapy. In one aspect, the method of determining the responsiveness of a subject having cancer to immunotherapy comprises, or consists essentially of, or yet further consists of contacting tissue-resident memory cells (TRMs) isolated from the subject, e.g., of the cancer or a sample thereof, with an antibody or agent that recognizes and binds one or more proteins encoded by a gene set forth in Table 1, Table 2, Table 3, Table 4, Table 5 and/or Table 7 and, optionally, an antibody or agent that recognizes and binds CD8, an antibody or agent that recognizes and binds PD-1, an antibody or agent that recognizes and binds TIM3, an antibody or agent that recognizes and binds LAG3, an antibody or agent that recognizes and binds CD28H (TMIGD2), an antibody or agent that recognizes and binds AMICA1, an antibody or agent that recognizes and binds KLF3, an antibody or agent that recognizes and binds S1PR5, an

antibody or agent that recognizes and binds S1PR1, an antibody or agent that recognizes and binds KLF2 and an antibody or agent that recognizes and binds CTLA4 to determine the frequency of TRMs expressing these proteins, wherein a high frequency of TRMs expressing these proteins indicates responsiveness to immunotherapy. For any of the methods disclosed herein, the TRMs may comprise, or consist essentially of, or yet further consist of CD19−CD20−CD14−CD56−CD4−CD45+CD3+CD8+ T-cells.

Further disclosed are methods of identifying a subject that will or is likely to respond to a cancer therapy, comprising, or consisting essentially of, or yet further consisting of contacting a sample isolated from the subject with an agent that detects the presence of one or more genes set forth in Table 1, Table 2, Table 3, Table 4, Table 5 and/or Table 7 in the sample, (e.g., cancer or a sample thereof), wherein the presence of the one or more genes at higher or lower than baseline expression levels indicates that the subject is likely to respond to cancer therapy. In one aspect, the baseline expression is normalized mean gene expression. In another aspect, the higher than baseline expression is at least about a 2-fold increase in expression relative to baseline expression and/or lower than baseline expression is at least about a 2-fold decrease in expression relative to baseline expression. Expression can be reduced or increased by at least about 2 or more, or about 3, or about 4, or about 5, or about 6, or about 7, or about 8, or about 9, or about 10, or about 11, or about 12, or about 13, or about 14, or about 15 fold as compared to a comparative wild-type cell. One of skill in the art can monitor expression of the genes using methods such as RNA-sequencing, DNA microarrays, Real-time PCR, or Chromatin immunoprecipitation (ChIP) etc. Protein expression can be monitored using methods such as flow cytometry, Western blotting, 2-D gel electrophoresis or immunoassays etc. The method may further comprise, or consist essentially of, or yet further consist of administering a cancer therapy to the subject. The cancer therapy or cytoreductive therapy can be chemotherapy, immunotherapy, radiation therapy, and/or administering to the subject or contacting the tumor with an effective amount of the modified T-cells and/or the composition of this disclosure.

The cancer, tumor, or sample can be contacted with an agent, optionally including a detectable label or tag. In one aspect, the detectable label or tag can comprise, or consist essentially of, or yet further consist of a radioisotope, a metal, horseradish peroxidase, alkaline phosphatase, avidin or biotin. In another aspect, the agent can comprise, or consist essentially of, or yet further consist of a polypeptide that binds to an expression product encoded by the gene, or a polynucleotide that hybridizes to a nucleic acid sequence encoding all or a portion of the gene. The polypeptide may comprise, or consist essentially of, or yet further consist of an antibody, an antigen binding fragment thereof, or a receptor that binds to the gene. In one aspect, the antibody is an IgG, IgA, IgM, IgE or IgD, or a subclass thereof. In another aspect, the IgG antibody is an IgG1, IgG2, IgG3 or IgG4. The antigen binding fragment can be a Fab, Fab′, F(ab′)2, Fv, Fd, single-chain Fvs (scFv), disulfide-linked Fvs (sdFv) or VL or VH. In one aspect, the agent is contacted with the cancer, tumor, or sample in conditions under which it can bind to the gene it targets. The contacting can be performed in vitro, or alternatively in vivo. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

The methods of this disclosure the method comprise, or consist essentially of, or yet further consist of detection by immunohistochemistry (IHC), in-situ hybridization (ISH), ELISA, immunoprecipitation, immunofluorescence, chemiluminescence, radioactivity, X-ray, nucleic acid hybridization, protein-protein interaction, immunoprecipitation, flow cytometry, Western blotting, polymerase chain reaction, DNA transcription, Northern blotting and/or Southern blotting. The sample may comprise, or consist essentially of, or yet further consist of cells, tissue, an organ biopsy, an epithelial tissue, a lung, respiratory or airway tissue or organ, a circulatory tissue or organ, a skin tissue, bone tissue, muscle tissue, head, neck, brain, skin, bone and/or blood sample. In another aspect, the sample comprises one or more of sputum, serum, plasma, lymph, cystic fluid, urine, stool, cerebrospinal fluid, ascite fluid, blood, or a tissue. While the cancer or tumor described herein can be an epithelial, a head, neck, lung, lung, prostate, colon, pancreas, esophagus, liver, skin, kidney, adrenal gland, brain, or comprises a lymphoma, breast, endometrium, uterus, ovary, testes, lung, prostate, colon, pancreas, esophagus, liver, skin, kidney, adrenal gland and/or brain cancer or tumor, a metastasis or recurring tumor, cancer or neoplasia, a non-small cell lung cancer (NSCLC) and/or head and neck squamous cell cancer (HNSCC). In a further aspect, the methods of this disclosure may comprise, or consist essentially of, or yet further consist of detecting in the subject, the cells or the sample the number or density of Trm cells that are CD19−CD20−CD14−CD56−CD4−CD45+CD3+CD8+ T-cells.

### Kits

Finally, provided herein is a kit comprising, or consisting essentially of, or yet further consisting of one or more of the modified T-cells and/or the composition of this disclosure and instructions for use. In one particular aspect, the present disclosure provides kits for performing the methods of this disclosure as well as instructions for carrying out the methods of the present disclosure.

The kits are useful for detecting the presence of cancer such as B-cell lymphoma in a biological sample e.g., any bodily fluid including, but not limited to, e.g., sputum, serum, plasma, lymph, cystic fluid, urine, stool, cerebrospinal fluid, acitic fluid or blood and including biopsy samples of body tissue. The test samples may also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kit components, (e.g., reagents) can be packaged in a suitable container. The kit can also comprise, or alternatively consist essentially of, or yet further consist of, e.g., a buffering agent, a preservative or a protein-stabilizing agent. The kit can further comprise, or alternatively consist essentially of, or yet further consist of components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

### Modes for Carrying Out the Disclosure

Using single-cell and bulk transcriptomic analysis of purified populations of TRM and non-T_{RM} cells present in tumor and normal lung tissue from patients with lung cancer, Applicants identified a distinct population of highly functional TRM cells present exclusively in the tumors. These TRM cells proliferate, display clonal expansion and express high levels of TIM3, CXCL13 and CD39. They also expressed high levels of PD-1 but show no features of exhaustion. Rather, these ‘highly functional’ TRM cells are the key cell types contributing to the robust anti-tumor responses induced by PD-1 inhibitors in some cancer patients. Because PD-1 expression was also observed in TRM cells in the normal lung, without being bound by theory, Applicant believes that PD1 inhibitors may have the potential to non-specifically reactivate quiescent TRM cells present in normal lung and presumably other tissues and cause the clinically recognised immune-related toxicities. These findings have implications for the design of therapies that preferentially activate “highly functional” TRM cells in tumors while minimizing toxicity.

In lung cancer and many other solid tumors, the presence of an adaptive anti-tumor immune response is positively correlated with patient survival.′ This response is mediated primarily by CD8⁺cytotoxic T lymphocytes (CTLs). Because CTLs in tumors are chronically activated, they can become “exhausted,” a hyporesponsive state, that prevents inflammatory damage to healthy tissue in the setting of infection.² Exhaustion involves up-regulation of surface inhibitory molecules, such as PD-1 and TIM3.³ PD-1 inhibitors have revolutionized cancer treatment by inducing durable responses in some patients.⁴ Given the association of PD-1 with exhaustion and the description of CTLs expressing PD-1 in human cancers, exhausted CTLs are generally assumed to be the cells reactivated by anti-PD-1 therapy, though definitive evidence for this is lacking in humans.⁵

Though PD-1 inhibitors can eradicate tumors in some cancer patients, they also lead to serious adverse immune-mediated reactions,⁶ calling for research to identify features unique to tumor-reactive CTLs. One subset of CTLs that may harbor such distinctive properties are tissue-resident memory T cells (T_{RM}), which mediate the response to anti-tumor vaccines' and facilitate rejection of tumors in animal models.⁸ TRM responses have also recently been shown by Applicant⁹ and others¹⁰ to associate with better survival in human solid tumors. The molecular features of TRM cells' response has been characterized in the setting of infection and involves rapid clonal expansion and upregulation of molecules aiding recruitment and activation of additional immune cells, alongside the traditional effector functions of CTL.¹¹ However, the molecular features that drive the anti-tumor functions of human TRM cells was previously unknown. To address this question, the Applicants compared the transcriptome of TRM and non-T_{RM} CTLs present in tumor and normal lung tissue samples.

### CD103 Expressing CTLs in Human Lungs are Enriched for Core Tissue Residency Features

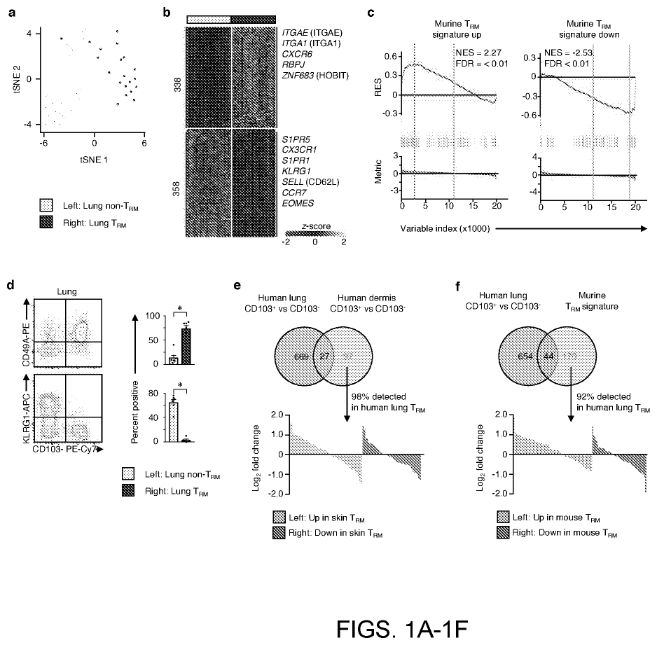

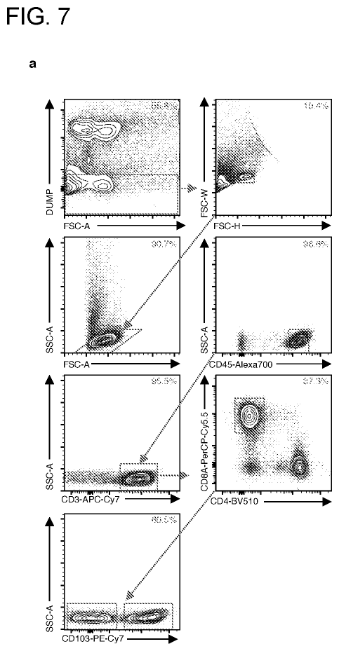

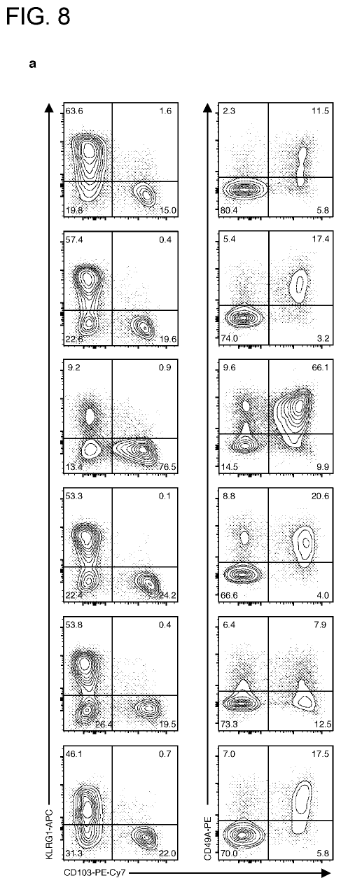

CTLs were isolated from lung tumor and adjacent uninvolved lung tissue samples provided by patients (n=30) with treatment-naïve early-stage non-small cell lung cancer (NSCLC), then sorted according to CD103 expression to separate TRM from non-T_{RM} cells (FIG. 7). The transcriptomes of each population were determined by RNA sequencing (RNA-Seq). Unbiased visualization of RNA-seq data of CTLs from normal lung using 2D t-stochastic neighbor embedding (tSNE) revealed the distinct nature of CD103⁺and CD103⁻ CTLs (FIG. 1A); nearly 700 transcripts were differentially expressed between the two populations (FIG. 1B and Table 3). Transcripts expressed at higher levels in CD103⁺ CTLs included several previously linked to TRM phenotype, such as S1PR1, S1PR5, ITGA1, RBPJ^{12,13}. Gene set enrichment analysis (GSEA) of lung CD103⁺ CTLs showed that the pattern of these transcripts' expression correlated with a core tissue residency signature¹⁴, previously defined by integration of transcriptomic datasets generated from murine CD8⁺ TRM cells isolated from several organs (FIG. 1C). The Applicants confirmed that lung CD103⁺ CTLs express CD49A¹³, an established T_{RM} molecule, and do not express KLRG1, linked to effector cells¹³, at the protein level (FIG. 1D and FIG. 8). Together, these data confirm that CD103⁺ CTLs in human lungs are highly enriched for T_{RM} cells; for simplicity, hereafter CD103⁺ CTLs are referred to as TRM cells and CD103⁻ CTLs as non-T_{RM} cells.

T_{RM} Cells in Human Lungs are Transcriptionally Distinct from Previously Characterized TRM Cells

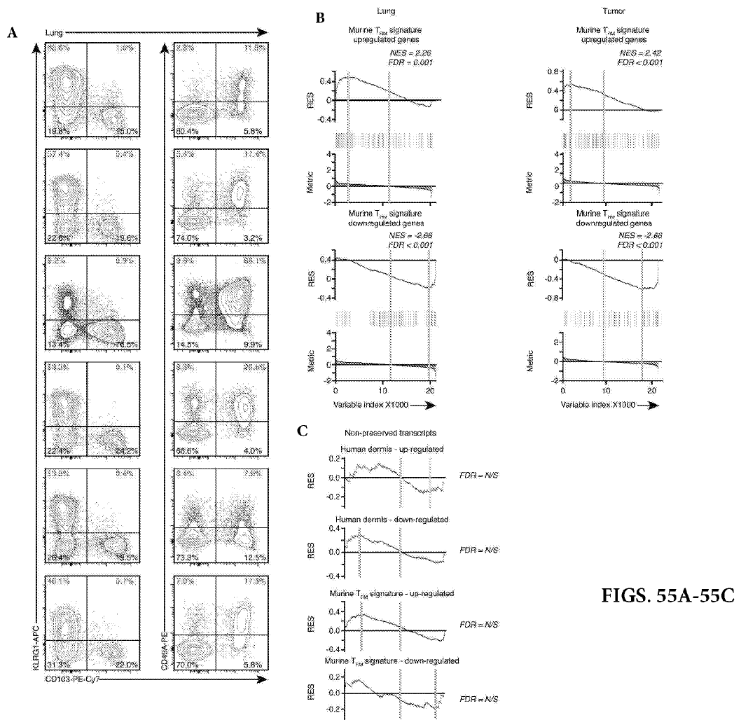

Differentially expressed transcripts between lung CD103⁺and CD103⁻ CTLs were compared with those reported for other TRM cells. The comparison with human skin TRM cells¹⁵ revealed limited overlap; the majority of transcripts differentially expressed in skin TRM cells relative to other CTLs were not differentially expressed between lung TRM and non-TRM cells (FIG. 1E). Similarly, comparisons with gene signatures of murine TRM cells isolated from multiple organs¹⁴ revealed limited overlap (FIG. 1E, FIG. 1F), although core tissue-residency features were well preserved. However, those differentially expressed transcripts that were not preserved across organs, or species, were not significantly enriched (FIG. 55). Thus, the transcriptional program, outside of a core tissue residency program of human lung TRM cells is quite distinct from that of human skin TRM cells and murine TRM cells present in several organs, and importantly, many of the features observed in human lung TRM cells have not been previously reported (Table 3)¹³.

### T_{RM} Cells in Normal Lung and Lung Tumors Share Tissue Residency Features, but are Otherwise Distinct

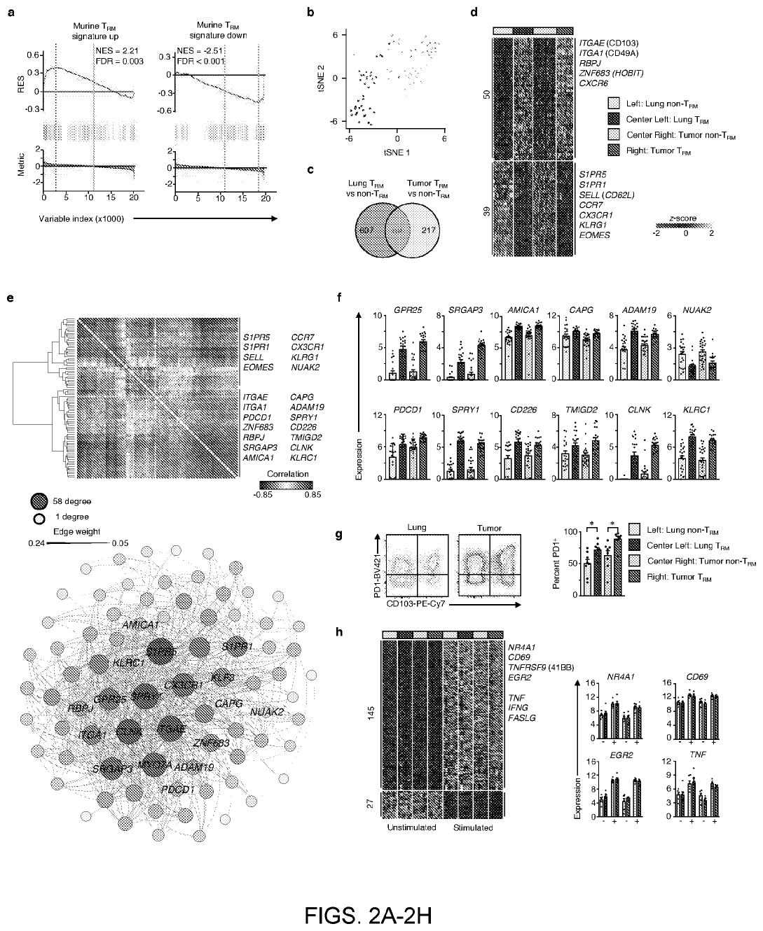

The Applicants analyzed whether TRM cells in lung tumors share tissue residency features with TRM cells in adjacent normal lung tissue. Gene set enrichment analysis (GSEA) of lung tumor-infiltrating CD103⁺ CTLs showed that their transcript expression correlated with the core murine tissue residency signature¹⁴, implying that even in tumors, CD103 expression defines TRM cells (FIG. 2A). Furthermore, over 300 transcripts were differentially expressed between CD103⁺and CD103⁻ CTLs present in lung tumors, and these included several transcripts previously linked to TRM cells (Table 4). However, CD103⁺and CD103⁻ CTLs from normal lung and tumor clustered separately (as 4 subpopulations) on tSNE plots (FIG. 2B). Nearly two-thirds of the T_{RM} properties, i.e., transcripts differentially expressed between CD103⁺and CD103⁻ CTLs, in tumors were different from those of normal lung TRM cells (FIG. 2C and Table 4).

Standard and weighted co-expression analysis (Methods) of the 89 ‘shared tissue residency’ transcripts (FIG. 2D) revealed a number of novel genes whose expression was highly correlated with known tissue residency (T_{RM}) genes, showing that their products play important roles in the development, trafficking or function of lung tumor-infiltrating TRM cells (FIG. 2E, FIG. 2F). Notable examples encoding products functioning in tumor TRM migration or retention include GPR25, SRGAP3, AMICA1, CAPG, ADAM19, and NUAK2 (FIG. 2E-2F).

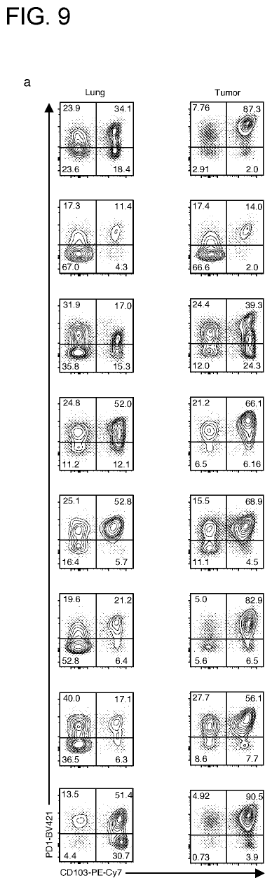

Another ‘shared tissue residency’ transcript was PDCD1, encoding PD-1 (FIG. 2E-2F). The Applicants confirmed at the protein level that PD-1 is expressed at higher levels in both tumor and lung TRM cells compared to non-T_{RM} cells (FIG. 2G and FIG. 9). Although PD-1 expression is considered typical of exhausted T cells³, recent reports have suggested that high PD-1 expression is a tissue residency feature of brain TRM cells independent of antigen stimulation^{16,17}, and of murine TRM cells from multiple organ systems¹⁴. In support of the conclusion that high expression of PD-1 reflects tissue residency rather than exhaustion, ex vivo stimulation of TRM and non-T_{RM} cells isolated from both lung and tumor tissue resulted in robust up-regulation of TCR-activation-induced genes (NR4A1, CD69, TNFRSF9 (4-1BB), EGR2) and cytokines (TNF, IFNG) (FIG. 2H). In addition to PDCD1, ‘shared tissue-residency’ transcripts included several (SPRY1¹⁸, CD226¹⁹, TMIGD2²⁰, CLNK²¹, KLRC1²²) that encode products reported to play a regulatory role in other immune cell types (FIG. 2F lower panel). The expression of these inhibitory molecules restrains the functional activity of tumor TRM cells.

### Tumor TRM Cells Proliferate, Express the Inhibitory Checkpoint TIM3 and Markers of Enhanced Function

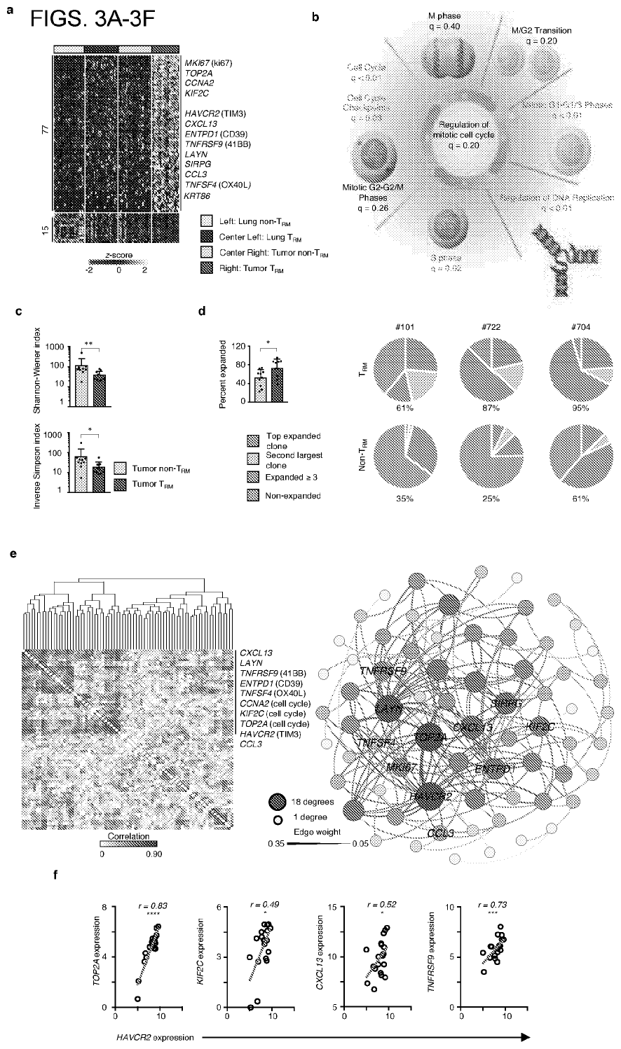

To identify features unique to tumor TRM cells, the Applicants compared their transcriptome to those of lung TRM cells and non-T_{RM} cells in both normal lung and tumors and detected 93 differentially expressed transcripts (FIG. 3A and Table 5). Reactome pathway analysis of ‘tumor T_{RM}-enriched’ transcripts showed significant enrichment for transcripts encoding components of the canonical cell cycle, mitosis and DNA replication machinery (FIG. 3B). The tumor TRM subset thus appears to be highly enriched for proliferating CTLs, presumably responding to tumor-associated antigens (TAA). Unique molecular identifier (UMI)-based T cell receptor (TCR) sequencing assays revealed a more restricted TCR repertoire in TRM cells compared to non-T_{RM} cells in tumors, as shown by significantly lower Shannon-Wiener and Inverse Simpson diversity indices (FIG. 3C and Table 6). Furthermore, the tumor TRM population contained a higher percentage of expanded clonotypes (73% vs. 52% in tumor TRM vs. non-T_{RM} populations) (FIG. 3D). The top expanded clonotype in each patient comprised, on average, 19% of all the clonotypes detected in TRM cells (FIG. 3D and Table 6), showing marked expansion of a single TAA-specific T cell clone in the tumor TRM population. In most patients, some expanded TCR clonotypes detected in the tumor TRM population were shared with cells in the non-T_{RM} population present in same tumor samples (Table 6), reflecting either derivation from common precursors or conversion of tumor TRM cells to effector non-T_{RM} cells.

‘Tumor T_{RM}-enriched’ transcripts that were highly correlated with cell cycle genes encode products with important functions and reflect the molecular features of TRM cells that are actively expanding in response to TAA. HAVCR2, encoding the co-inhibitory checkpoint molecule TIM3, was most correlated and connected with cell cycle genes (FIG. 3E-3F). TIM3 expression is a unique feature of lung tumor TRM cells that is not necessarily linked to exhaustion, as the other transcripts that correlated with expression of TIM3 and cell cycle genes encode molecules that could confer superior functionality such as CD39 (encoded by ENTPD1)²³, LAYN²⁴, CXCL13²⁵, CCL3²⁶, TNFSF4²⁷ (OX-40 ligand), as well as a marker of antigen-specific engagement (4-1BB, encoded by TNFRSF9) (FIG. 3E-3F)²⁸. Robust expression of this set of molecules was observed in neither human lung TRM cells nor in the mouse TRM signature, indicating that the tumor TRM population contains novel cell subsets.

### Single-Cell Transcriptomic Analysis Reveals Previously Uncharacterized TRM Subsets

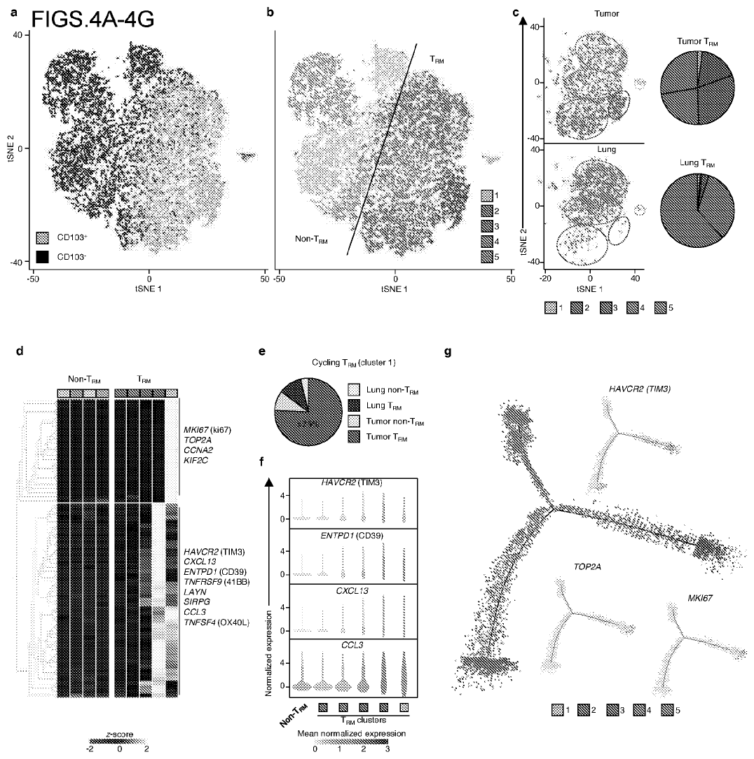

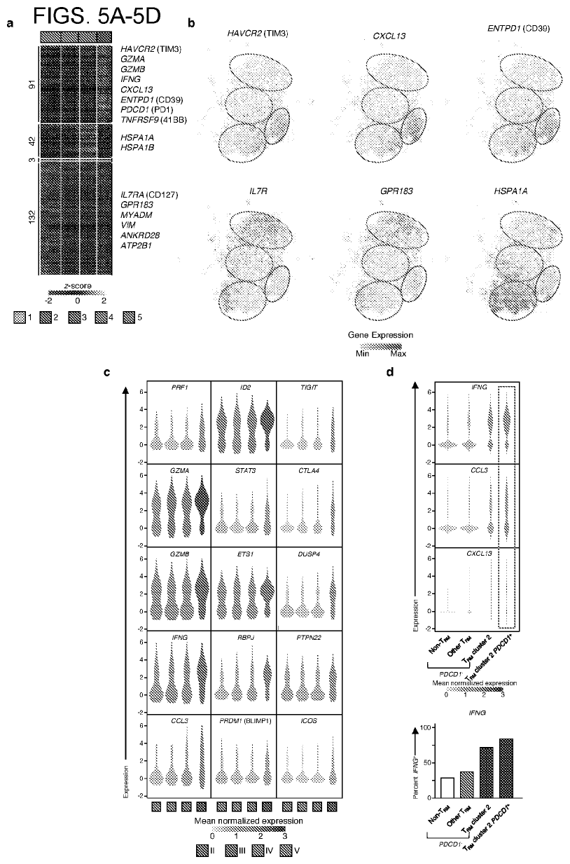

To determine whether ‘tumor T_{RM}-enriched’ transcripts are expressed in all or only a subset of the tumor TRM population, the Applicants performed single-cell RNA-Seq assays in CD103⁺ and CD103⁻ CTLs isolated from tumor and adjacent normal lung tissue from 12 patients with early-stage lung cancer. Analysis of the ˜12,000 single-cell transcriptomes revealed 5 clusters of TRM cells and 4 clusters of non-T_{RM} cells (FIG. 4A, FIG. 4B). Among the 5 TRM clusters, a greater proportion of cells in the tumor TRM population compared with the lung TRM population was observed in clusters 1-3, while clusters 4 and 5 contained more lung TRM cells (FIG. 4B-4C). Most strikingly, clusters 1-3 contained very few lung TRM cells (FIG. 4C). The ‘tumor T_{RM}-enriched’ transcripts detected in Applicants' analysis of bulk populations (FIG. 3A) were contributed by cells in these subsets.

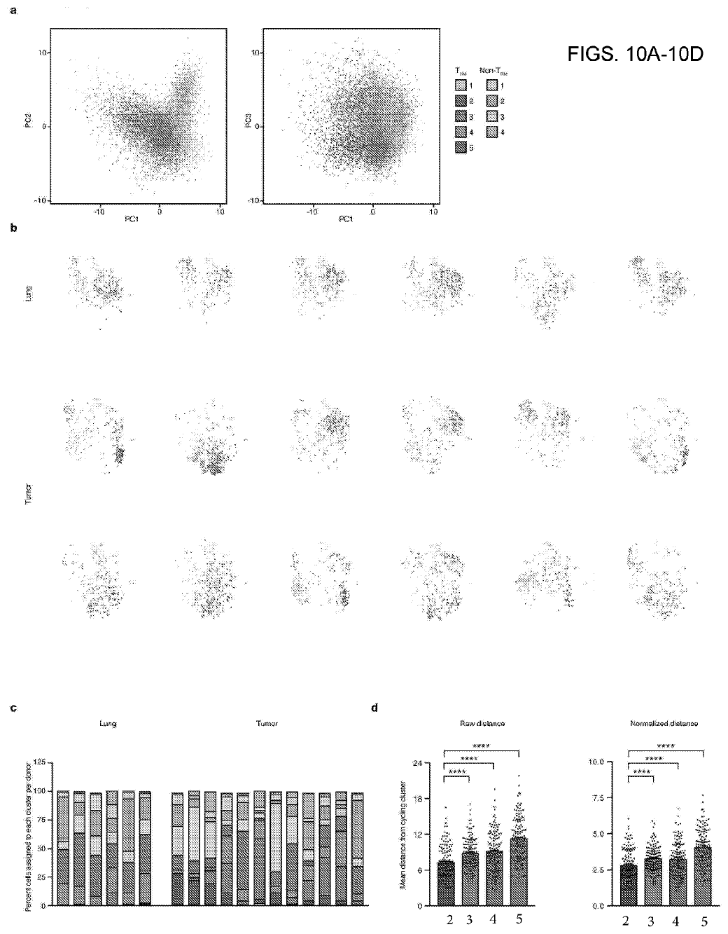

In agreement with that conclusion, cells in cluster 1 expressed high levels of the 25 cell cycle-related ‘tumor T_{RM}-enriched’ transcripts (FIG. 4D)²⁹, indicating that the enrichment of cell cycle transcripts in the bulk tumor T_{RM} population was contributed by this relatively small subset. Because these cells are actively proliferating, they represent TAA-specific cells. The majority of cells in this cycling cluster were from the tumor TRM population (FIG. 4E). These cells, along with those in the larger cluster 2, were highly enriched for other prominent ‘tumor T_{RM}-enriched’ transcripts like HAVCR2 (TIM3), including those encoding products that could confer superior functionality (e.g., CD39, LAYN, CXCL13, CCL3; FIG. 4F). This shared expression pattern shows that the cycling cluster simply represent cells in cluster 2 that are entering the cell cycle. Confirming this idea, cell-state hierarchy maps of all tumor TRM cells, constructed using Monocle2³⁰, revealed that cells in cluster 2 were most similar to the cycling TRM cells (cluster 1) (FIG. 4G and FIG. 10). Additionally, the Applicants found that when performing hierarchical clustering of these cells, the proliferating cluster 1 clustered more with cells assigned to cluster 2 than the other TRM clusters (FIG. 4F). This finding was corroborated when Applicants calculated the average distance in principle component space between each cell in cluster 1 to the other TRM clusters (FIG. 10D). Overall, the single-cell transcriptome analysis uncovered additional distinct subsets of tumor TRM cells that have not previously been described and play an important role in anti-tumor immune responses.

### A Subset of Tumor TRM Cells has a Transcriptional Program Indicative of Superior Functional Properties

To dissect the molecular properties unique to tumor-infiltrating TRM cells in each of the 4 larger clusters, the Applicants performed multiple pair-wise single-cell differential gene expression analyses (Methods). Over 250 differentially expressed genes showed higher expression in any one of the Applicants' clusters (FIG. 5A and Table 7), indicating that cells in different clusters had divergent gene expression programs. For example, cells in cluster 3 were highly enriched for transcripts encoding heat shock proteins (e.g., HSPA1A, HSPA1B and HSP90AA1), whereas cells in cluster 5, comprising TRM cells from normal lung and tumor tissue, expressed high levels of IL7R, which encodes the IL-7 receptor, a marker of memory precursor cells³¹, and transcripts such as GPR183³², MYADM³³, VIM³⁴ and ANKRD28³⁵, which encode proteins involved in cell migration and tissue homing (FIG. 5A, FIG. 5B).

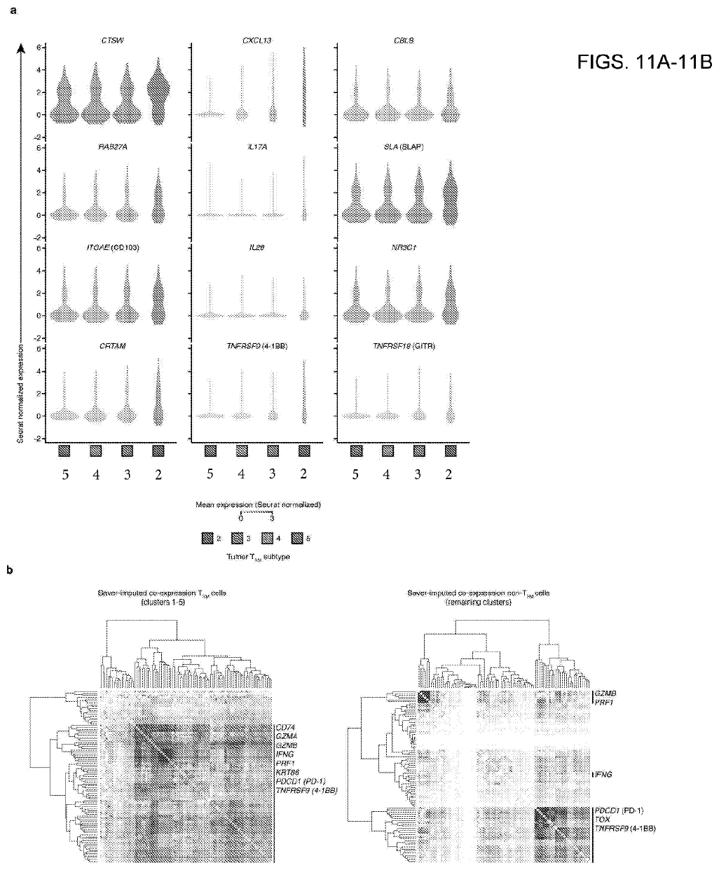

Because of their close relationship with cycling TRM cells (FIG. 4D, FIG. 4G), the Applicants' analysis focused on TRM cells in cluster 2. The 91 transcripts expressed more highly by these cells than other TRM clusters (FIG. 5A) included several with encoded products linked to cytotoxic activity such as PRF1, GZMB, GZMA, CTSW³¹, RAB27A³⁶, ITGAE³⁷ and CRTAM³¹ (FIG. 5C and FIG. 11), as well as a number encoding effector cytokines and chemokines, such as IFN-γ, CCL3, CXCL13, IL17A and IL26. Cluster 2 also expressed high levels of transcripts encoding transcription factors known to promote the survival of memory or effector CTLs (ID2³⁸, STAT3³⁹, ZEB2⁴° and ETS-1⁴¹) or that are involved in establishing and maintaining tissue residency (RBPJ, a key player in Notch signaling¹³, and BLIMP1⁴², encoded by PRDM1) (FIG. 5C and FIG. 11). TRM cells in cluster 2 also highly expressed ENTPD1 (FIG. 5B, FIG. 5C), which encodes CD39, an ectonucleotidase that cleaves ATP, which may protect this TRM subset from ATP-induced cell death in the ATP-rich tumor microenvironment²³. This expression pattern confers highly effective and sustained anti-tumor immune function; in combination with earlier results, it was determined that this ‘highly functional’ TRM subset represents TAA-specific cells that proliferate in tumors.

T_{RM} cells in cluster 2 expressed the highest levels of PDCD1 transcripts (FIG. 5A) and were enriched for transcripts encoding other molecules linked to exhaustion such as TIM3, TIGIT¹⁹, and CTLA4³, and inhibitors of TCR-induced signaling and activation like CBLB, SLAP, DUSP4, PTPN22 and NR3C1 (glucocorticoid receptor) (FIG. 5A-5C) and FIG. 11)⁴³⁻⁴⁶. Nonetheless, these TRM cells exhibited a transcriptional program suggestive of superior effector properties and cell proliferation, and expressed high transcript levels for several co-stimulatory molecules such as 4-1BB, ICOS and GITR (TNFRSF18) (FIG. 5C and FIG. 11)³. More specifically, PDCD1-expressing TRM cells in cluster 2 expressed relatively higher levels of IFNG, CCL3, and CXCL13 transcripts compared with cells not expressing PDCD1 in that cluster and other tumor-infiltrating TRM and non-T_{RM} cells (FIG. 5D). This co-expression program appeared to be specific to the tumor TRM compartment, given it was also reflected in a SAVER-imputed co-expression profiled being identified specifically in the TRM subsets, but not the non-TRM subsets (FIG. 11B). Overall, these findings agree with the bulk RNA-Seq analysis, indicating that inside this specific subset of CTLs expression of inhibitory molecules, like PD-1, does not reflect exhaustion. Instead, it prevents TCR-activation-induced cell death to sustain robust anti-tumor CTL responses^{23,47}.

### PD-1- and TIM3-Expressing Tumor-Infiltrating TRM Cells are not Exhausted

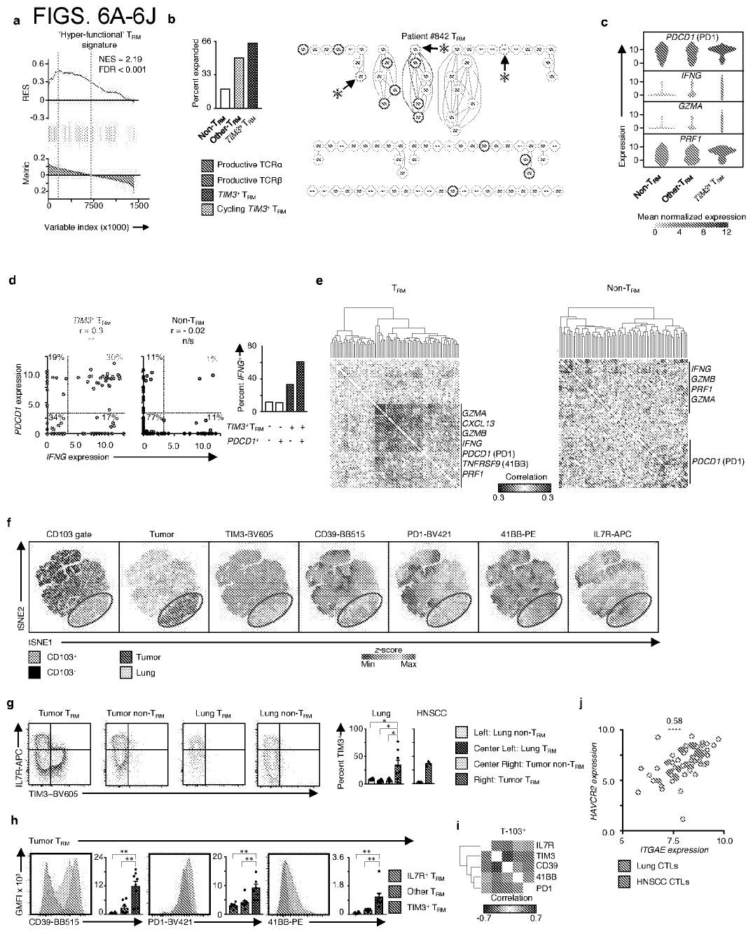

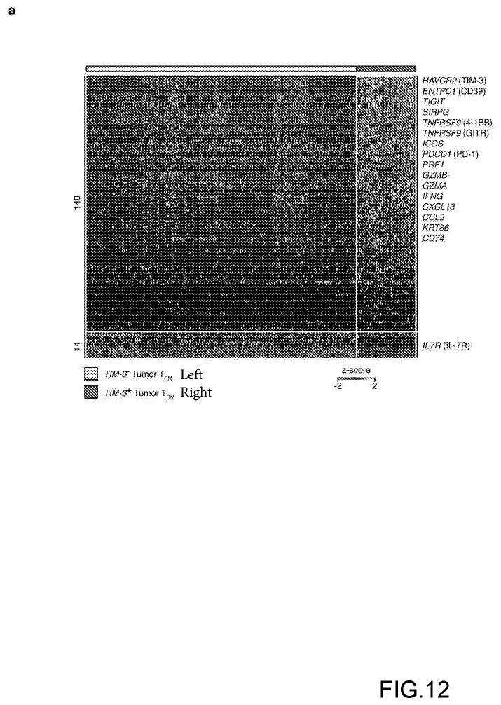

To further address whether PDCD1-expressing TRM cells in cluster 2 (highly functional ‘TRM cells’) were exhausted or functionally active, the Applicants performed single-cell RNA-seq in tumor-infiltrating TRM and non-T_{RM} cells, using SMART-seq2 for paired transcriptomic and TCR clonotype analysis³¹. The TCRβ chains (Methods) in 81% of single cells, the TCRα chain in 77%, and both chains in 70% of cells were reconstructed. As expected, clonally expanded tumor-infiltrating TRM cells, which are reactive to TAA, were significantly enriched for genes specific to ‘highly functional’ TRM cells (FIG. 6A). Among tumor-infiltrating CTLs, a greater proportion of TIM3-expressing (Methods) TRM cells were clonally expanded compared with other TRM and non-T_{RM} cells (FIG. 6B). As expected, TIM3-expressing TRM cells were significantly enriched for key effector cytokines and cytotoxicity transcripts, despite expressing higher levels of PDCD1 (FIG. 6C and FIG. 12). Importantly, it was discovered that a greater proportion of IFNG-expressing cells co-expressed PDCD1 among TIM3-expressing TRM cells compared with non-T_{RM} cells (FIG. 6D).

The higher sensitivity of the SMART-seq2 assay compared to the high-throughput 10× genomics platform also allowed better co-expression analysis due to lower dropout rates³¹. Co-expression analysis showed that expression of PDCD1 and HAVCR2 (TIM3) correlated with that of activation markers (TNFRSF9 and CD74), IFNG and cytotoxicity-related transcripts more strongly in TRM cells compared with non-T_{RM} cells (FIG. 6E). Specifically, IFNG and PDCD1 expression levels were better correlated in TIM3-expressing TRM cells compared with non-T_{RM} cells (FIG. 6D-FIG. 6E), and the proportion of cells strongly co-expressing these transcripts was notably higher (30% vs. 1%). Overall, these results strongly support that PD1 and TIM3 expressing tumor-infiltrating TRM are not exhausted, but instead are highly functional and are enriched for transcripts (IFNG, PRF1, GZMA) encoding for molecules linked to effector functions.

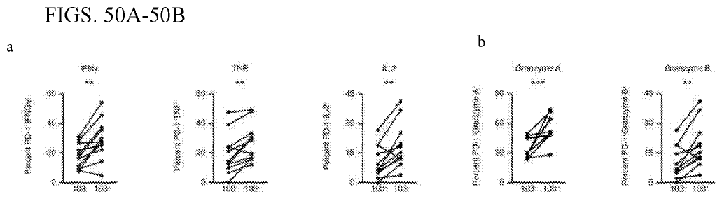

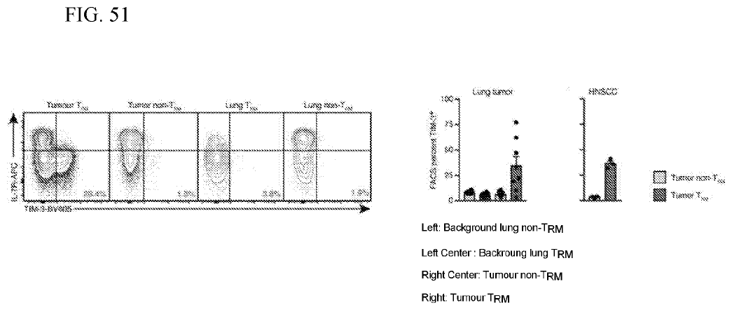

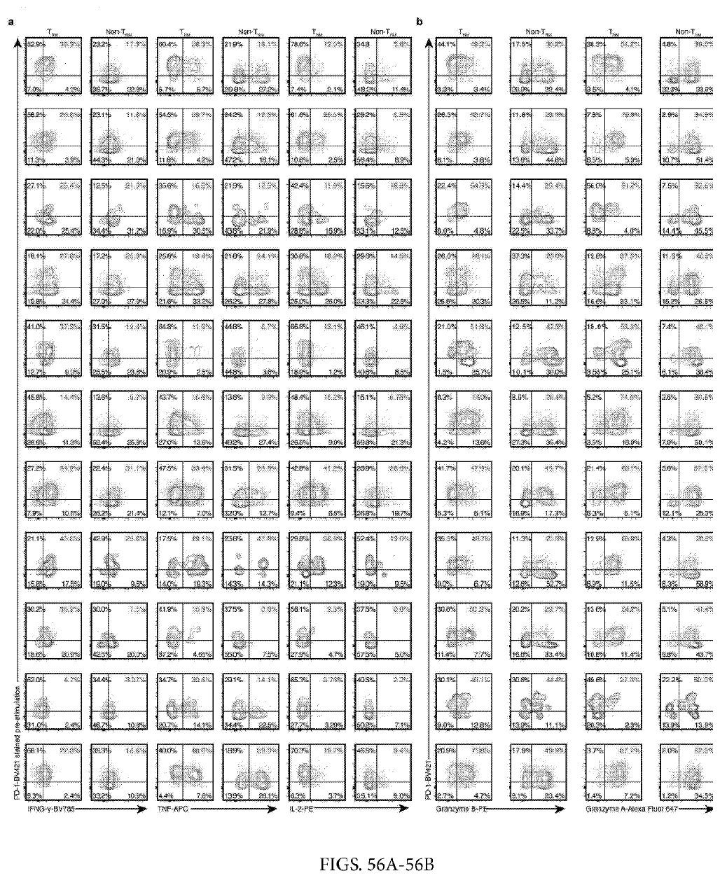

In keeping with the transcriptomic assays performed by Applicants, it was found that tumor-infiltrating TRM cells that co-expressed PD-1, when stimulated ex-vivo, had significantly higher percentage of cells expressing effector cytokines when compared to the non-TRM CTLs that co-expressed PD-1 (FIG. 50, FIG. 56A). Analysis directly ex-vivo demonstrated there was also greater expression of cytotoxic-associated proteins, granzyme A and granzyme B, in the PD-1+ TRM cells when compared to the PD-1+non-TRM CTLs in the tumor (FIG. 50, FIG. 56B). These data verify that PD-1 expression in the TRM subset of tumor-infiltrating CTLs does not reflect dysfunctional properties.

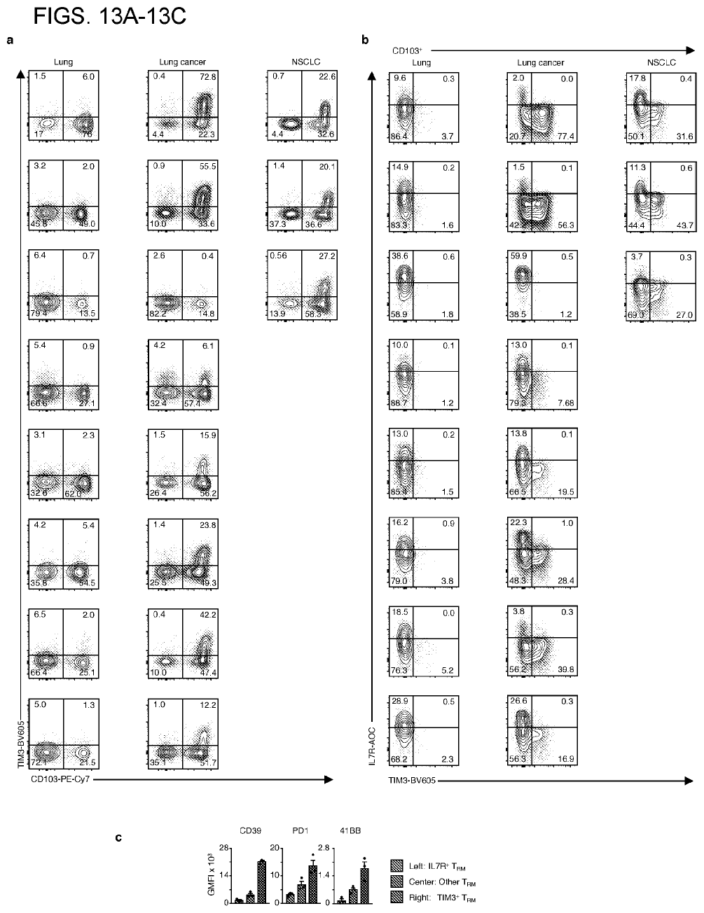

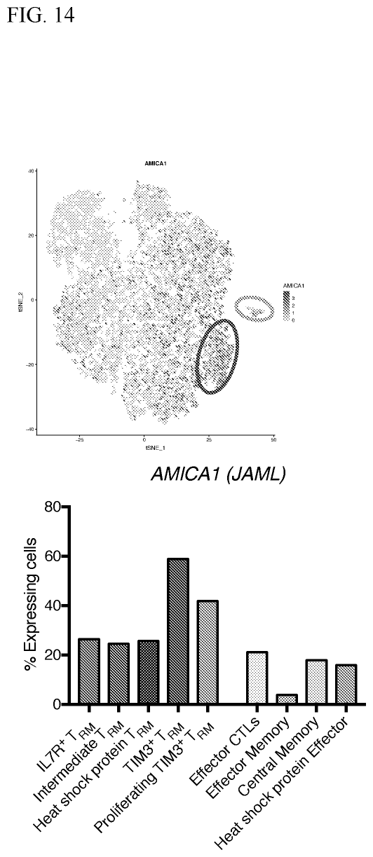

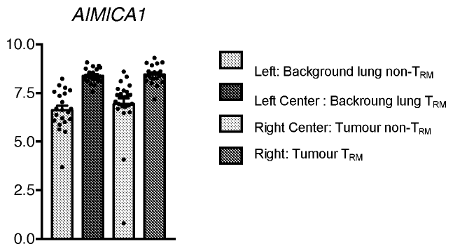

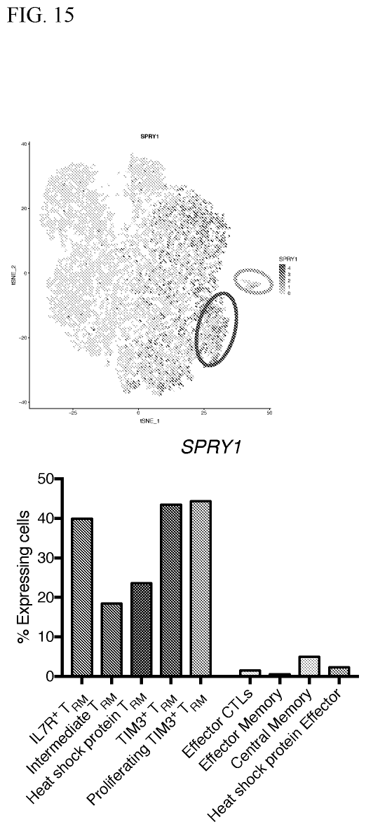

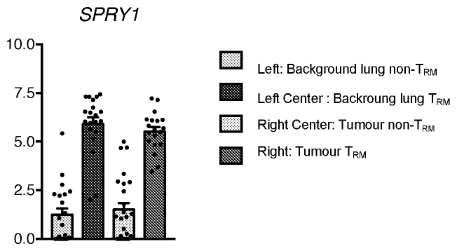

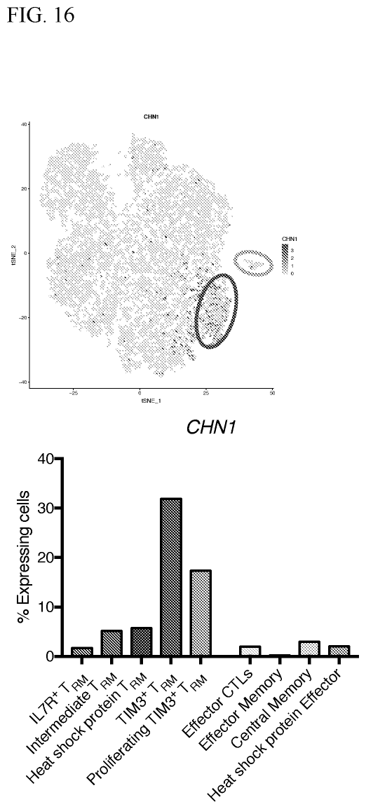

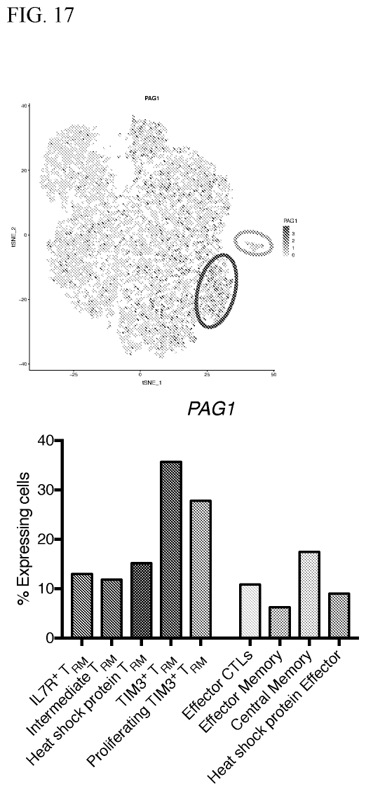

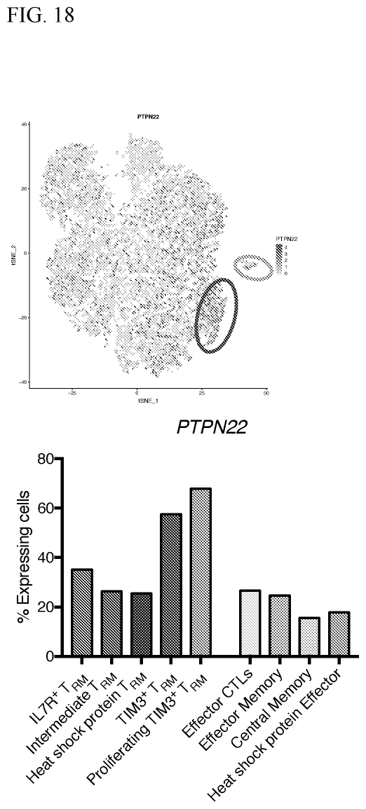

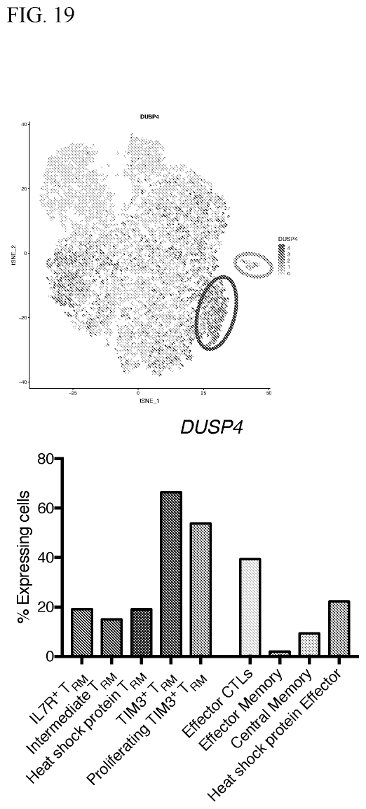

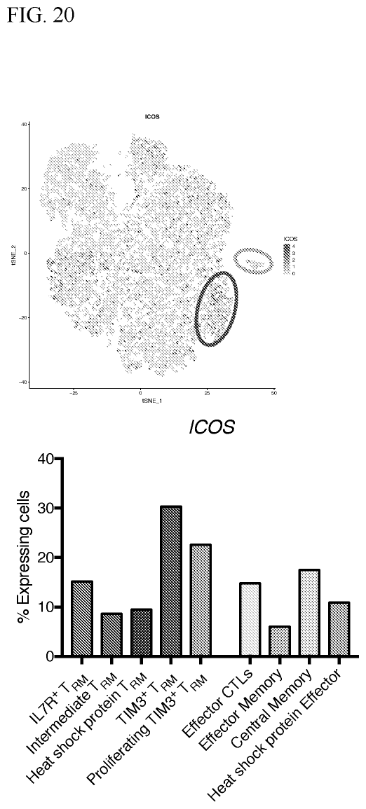

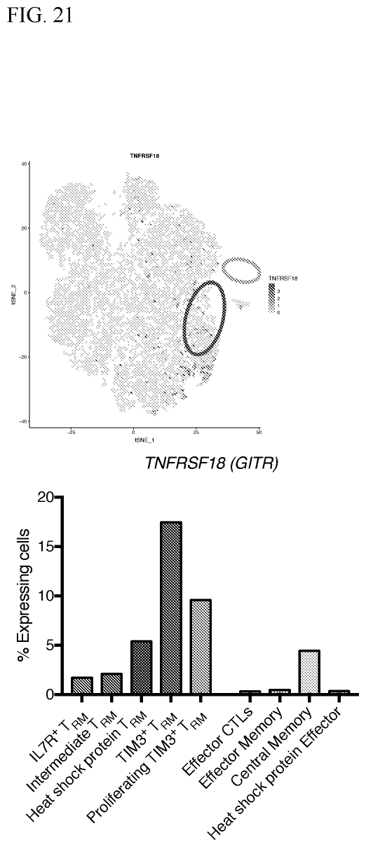

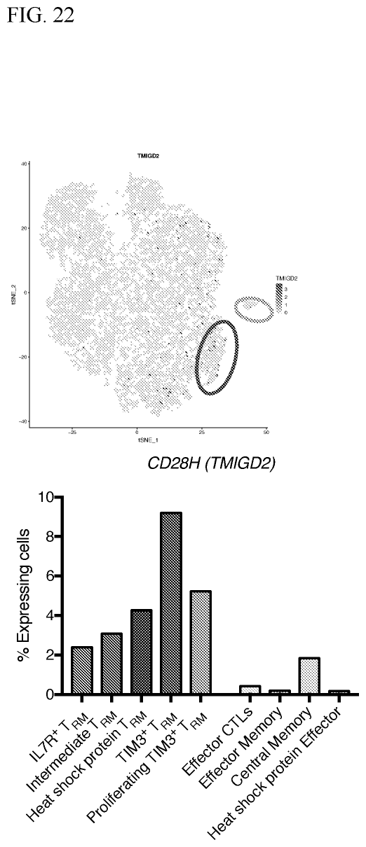

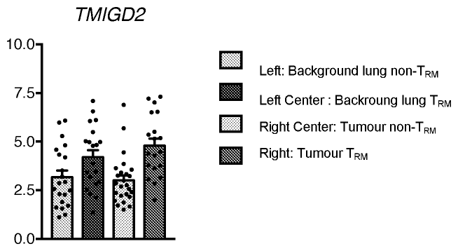

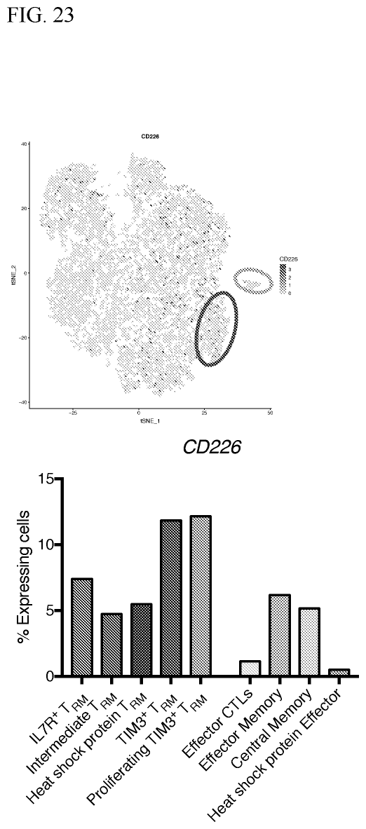

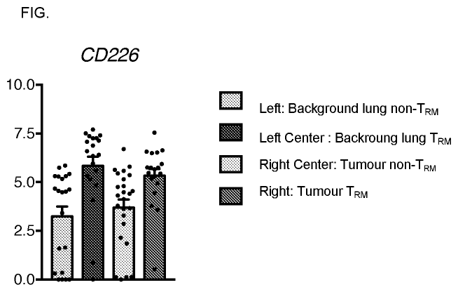

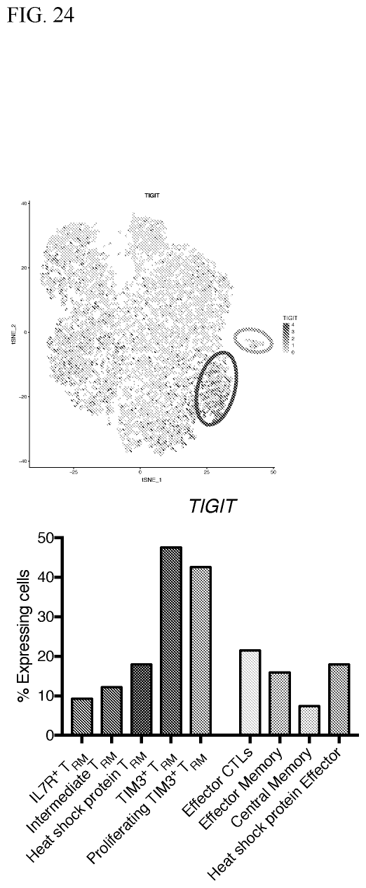

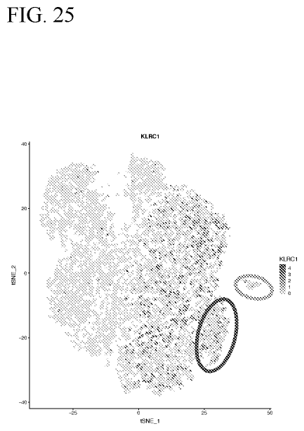

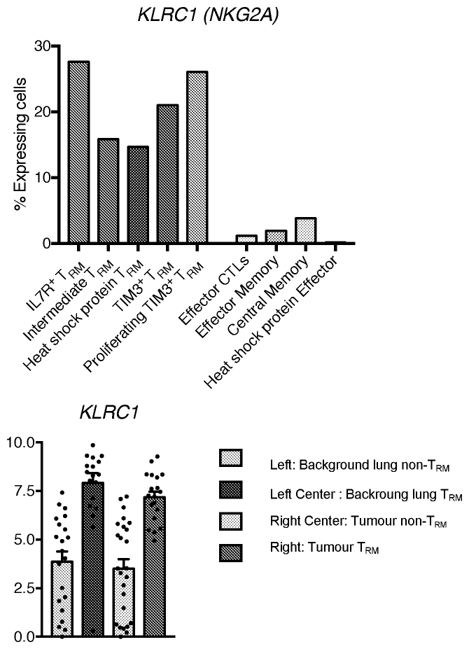

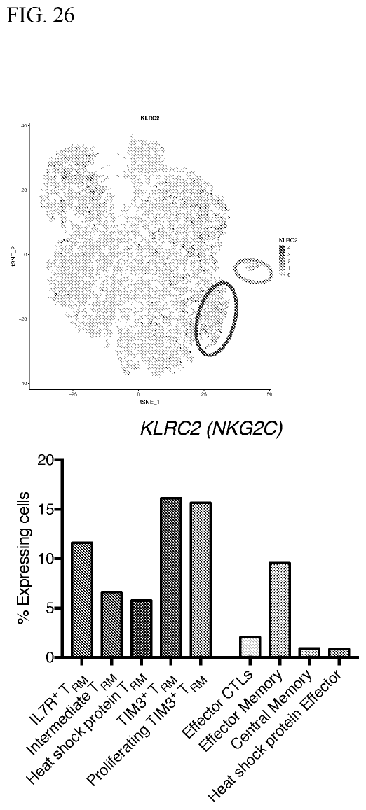

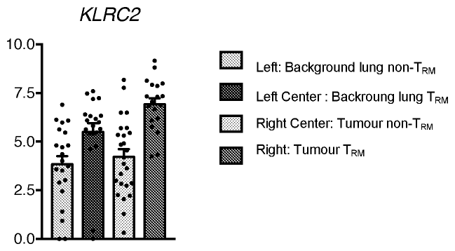

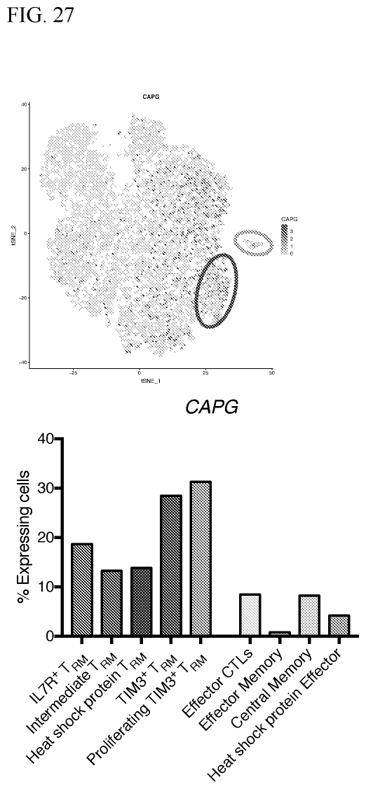

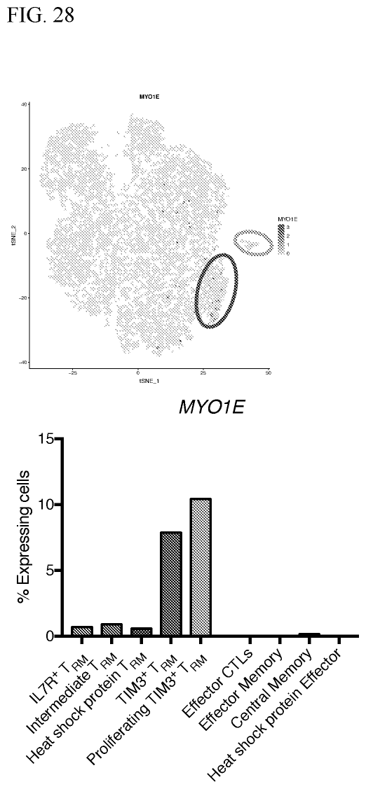

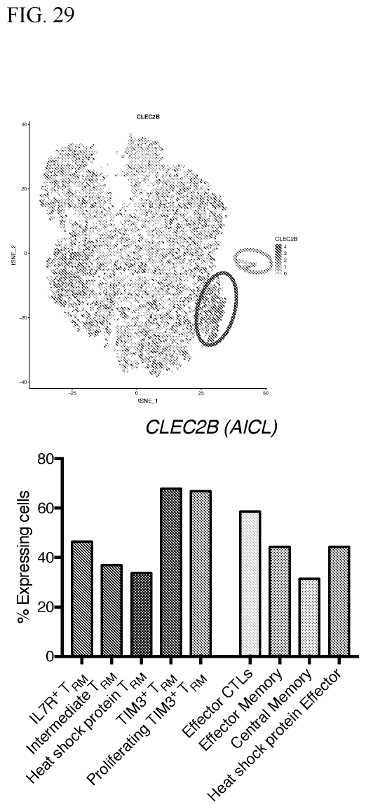

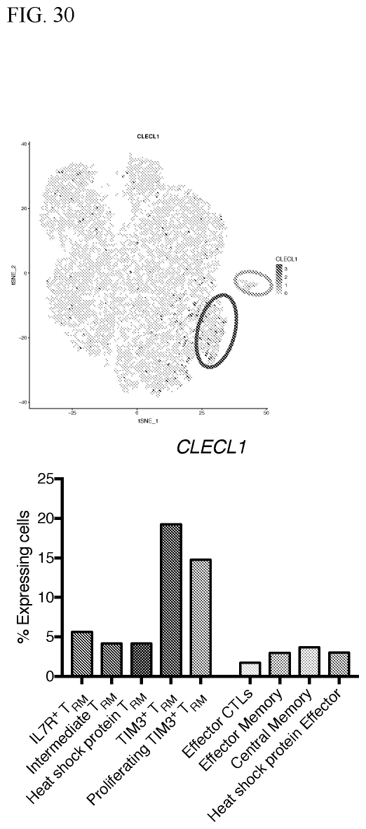

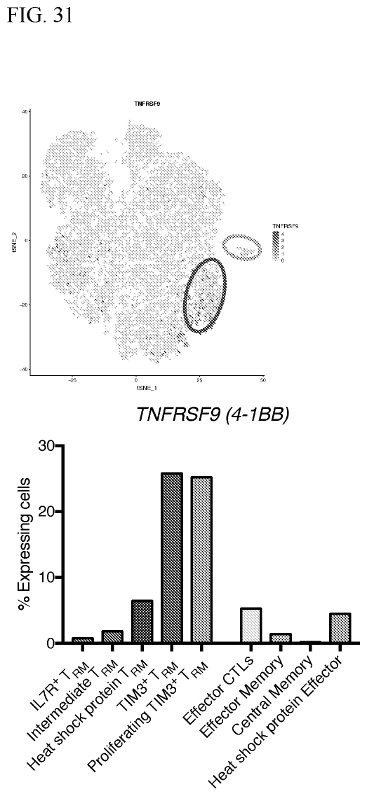

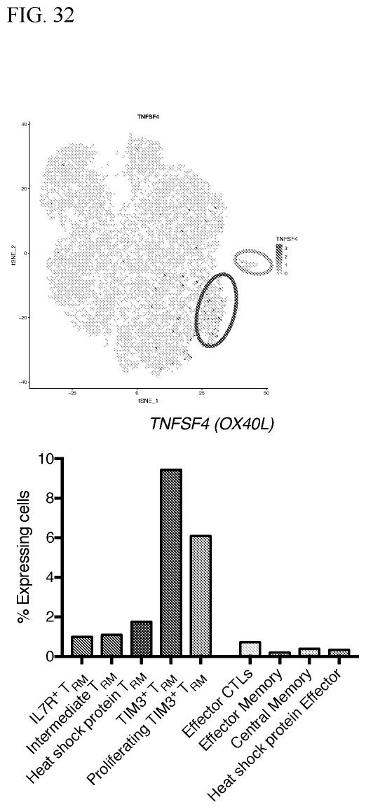

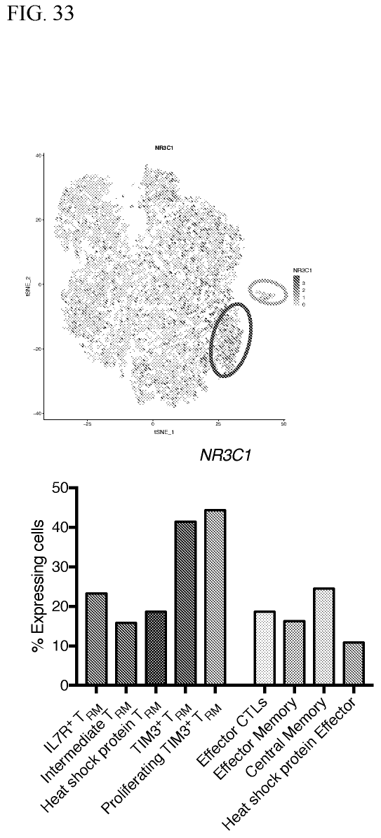

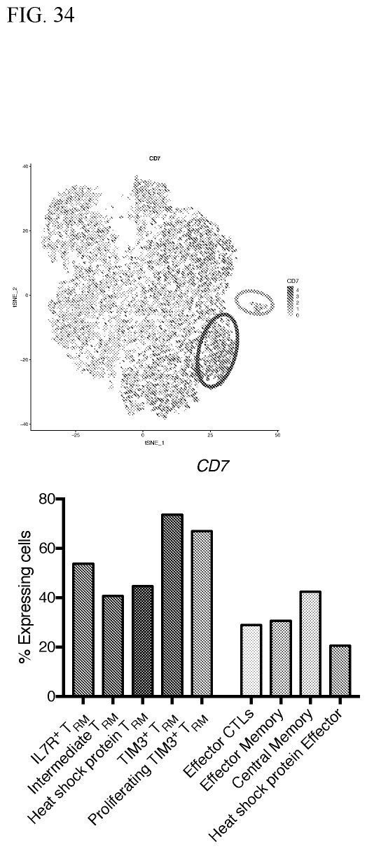

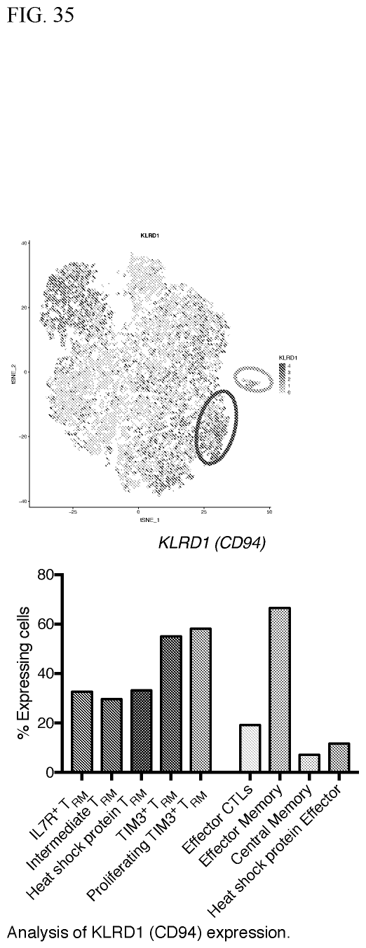

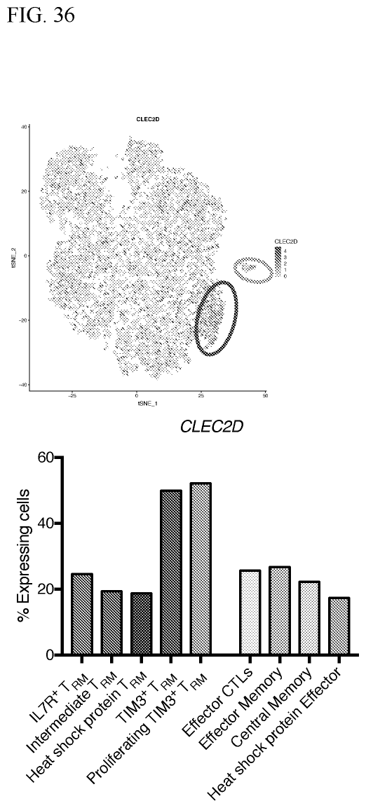

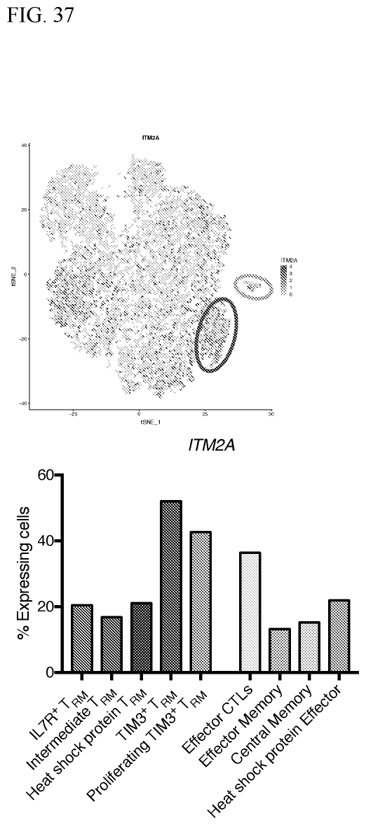

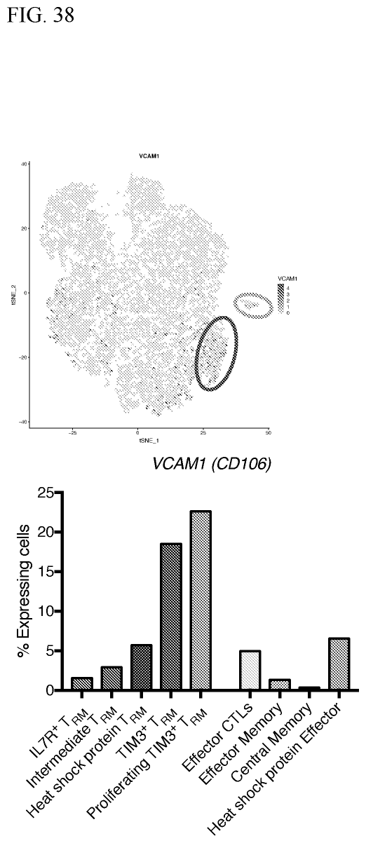

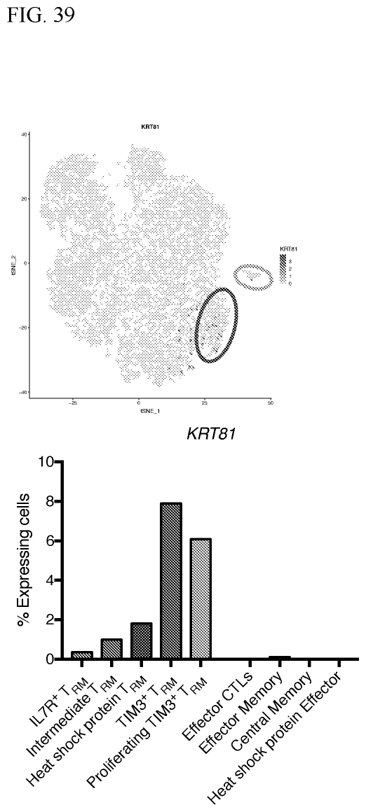

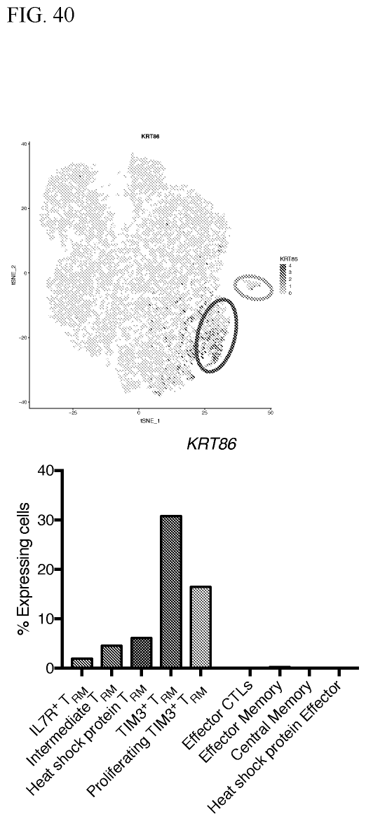

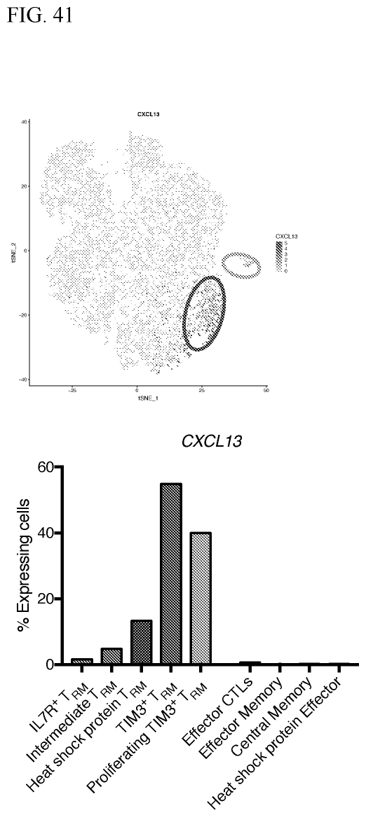

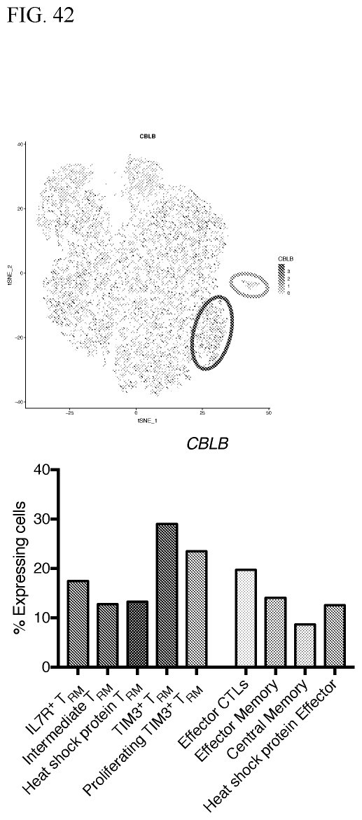

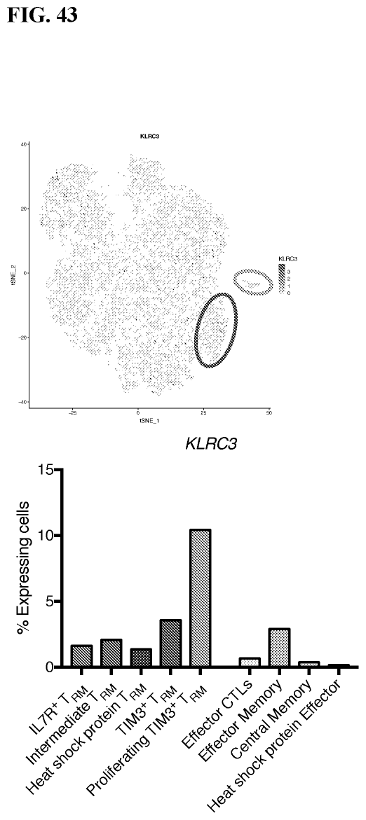

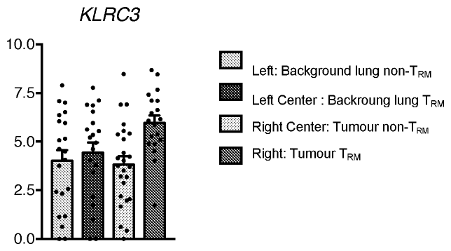

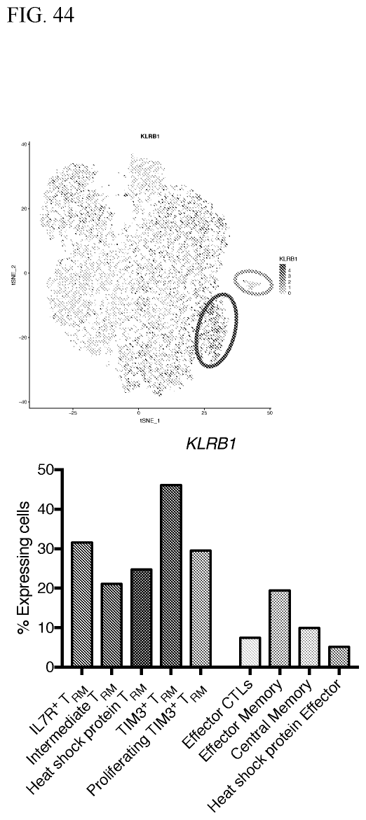

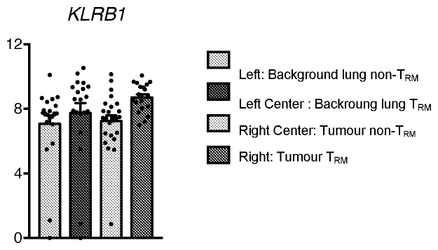

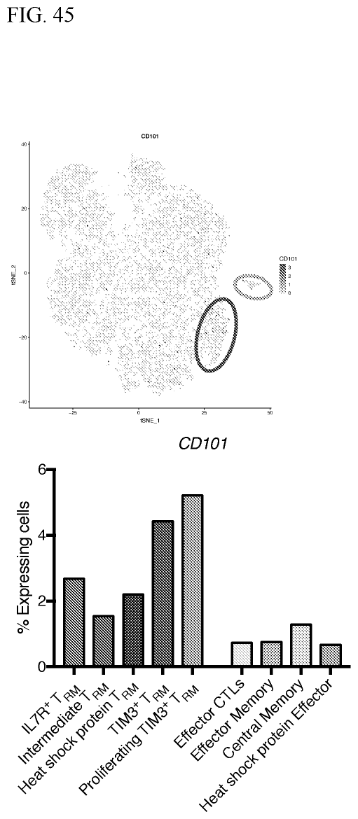

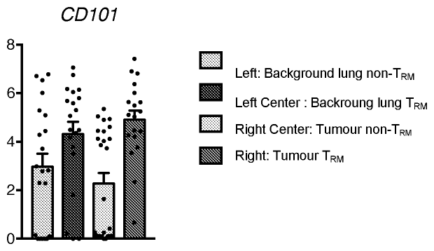

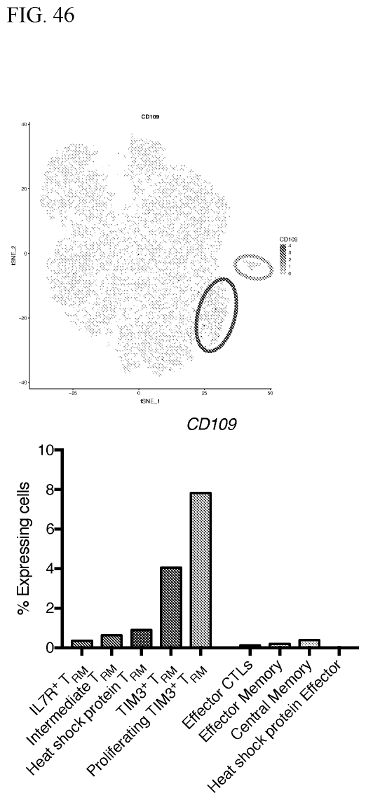

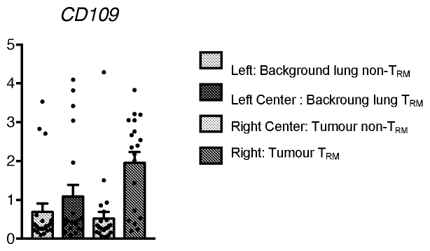

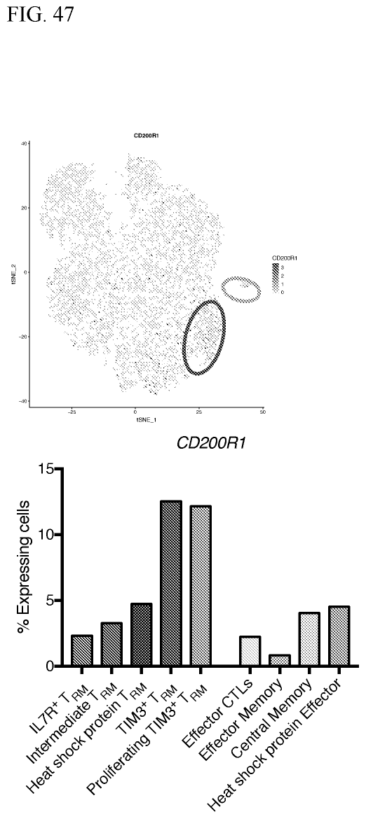

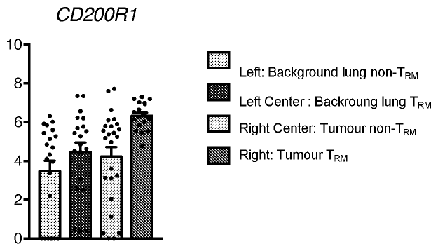

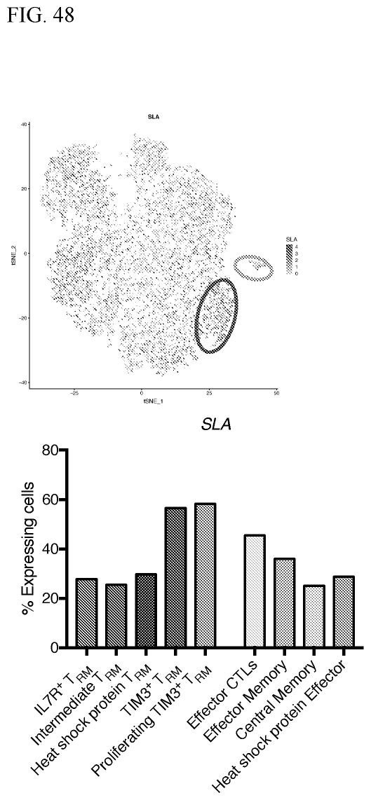

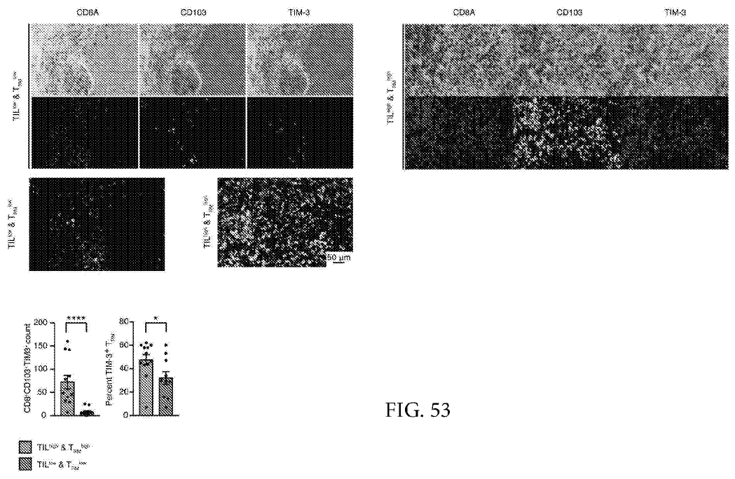

The Applicants evaluated the protein expression of selected molecules to better discern the tumor-infiltrating TRM subsets. Multi-parameter protein analysis of CTLs present in tumors and adjacent normal lung revealed a subset of TRM (CD103⁺) cells localized distinctly when the data was visualized in 2D space (FIG. 6F, left). This subset consisted of cells only from tumor tissue (circle, FIG. 6F), and uniquely expressed high levels of TIM3 and lacked IL-7R, indicating that this cluster is the same as the ‘highly functional’ TIM3-expressing TRM cluster (cluster 2) identified by single-cell RNA analysis (FIG. 6F, FIG. 6G and FIG. 13A). Consistent with the single-cell transcriptome analysis, the TIM3-expressing TRM cluster expressed higher levels of CD39, PD-1 and 4-1BB (FIG. 6F, FIG. 6H and FIG. 13B). PD-1 and TIM3 expression levels were also positively correlated with expression of 4-1BB, which is expressed following TCR engagement by antigen (FIG. 6I), indicating that these cells are highly enriched for TAA-specific cells. TIM3-expressing CTLs were detected among tumor-infiltrating TRM cells isolated from both lung cancer and head and neck squamous cell carcinoma (HNSCC) samples (FIG. 6G, right and FIG. 13B, FIG. 13C), but not among non-T_{RM} cells in these treatment naïve tumors or TRM cells in lung. Multi-color immunohistochemistry was used to confirm the presence of TIM-3-expressing TRM cells in lung tumor samples, which also showed enrichment of this subset in TIL^{hi}T_{RM}^{hi} “immune hot” tumors (FIG. 53 and Table 7). These findings confirm, at the protein level, the specificity of this ‘highly functional’ T_{RM} subset to tumors.

Given the highly specific expression of TIM3 in the subset of ‘highly functional’ tumor-infiltrating TRM cells, the TIM3 expression levels in the Applicants previous bulk CD8⁺ TIL transcriptome data⁹ was used as a surrogate to assess the relative magnitude of this ‘highly functional’ TRM subset in tumors, and thus relate this variable to features linked to better survival outcomes such as TRM density in tumors. The Applicants found a strong positive correlation between transcript levels of TIM3 and CD103 (ITGAE) in tumor-infiltrating CTLs (FIG. 6K), showing that tumors with high TRM density (high ITGAE levels) harbor more ‘highly functional’ TIM3-expressing TRM cells.

### Discussion

The disclosed bulk and single-cell transcriptomic analysis of lung and tumor-infiltrating TRM cells reveal that human TRM cells include at least 4 distinct subsets. Although human tumor-infiltrating TRM cells shared some core tissue residency features with those previously described from mouse models of infection and tumors, the vast majority of their molecular features were quite distinct. The most striking discovery was the identification of a ‘highly functional’ TIM3-expressing TRM subset present exclusively in tumors. This subset, although expressing high levels of PD-1 and other molecules previously thought to reflect exhaustion, exhibited a transcriptional program indicative of superior effector, survival and tissue residency properties and proliferated in the tumor milieu.

The Applicants defined a core set of genes commonly expressed in both lung and tumor TRM cells, including a number of novel genes whose expression was highly correlated with known tissue residency (TRM) genes. Any one of these genes may also be important for the development, trafficking or function of lung or lung tumor-infiltrating TRM cells. Some notable examples known or likely to have such functions are GPR25, whose closest homolog, GPR15⁴⁸, enables homing of T cell subsets to and retention in the colon; AMICA⁴⁹, encoding JAML (junctional adhesion molecule-like), which contributes to the proliferation and cytokine release of skin-resident γδT cells; and SRGAP, whose product functions in neuronal migration⁵⁰.

PDCD1 was a prominent hit in the ‘shared lung tissue residency’ gene list, and its expression was confirmed at the protein level in both lung and tumor TRM cells. The fact that PD-1 was expressed in TRM cells isolated from normal lung tissue of subjects with no active infection shows that PD-1 is constitutively expressed by human lung TRM cells, as has been recently described for brain TRM cells¹⁶. As PD-1 is expressed most highly by ‘highly functional’ TIM3-expressing tumor-infiltrating TRM cells, they may be the major cellular targets of anti-PD-1 therapy. Differences in the magnitude of this population of TRMs could thus be an explanation for the variation in the clinical response to PD-1 inhibitors, and non-responders may have defects in the de-novo generation of highly functional TIM3-expressing TRM cells. The constitutive expression of PD-1 by TRM cells in the normal lung and presumably other organs (skin, gut and pituitary gland) raises the possibility that anti-PD-1 therapy may non-specifically activate potentially self-reactive TRM cells to cause adverse immune reactions such as pneumonitis, dermatitis, colitis and hypophysitis⁶.

These findings raise the question of which molecular players are essential for the generation and maintenance of this novel ‘highly functional’ TIM3-expressing subset of TRM cells. This analysis identified a number of potential transcription factors (e.g., STAT3, ID2, ZEB2, ETS-1) and other molecules (e.g., PTPN22, DUSP4, LAYN, KRT86, CD39) that are uniquely expressed in this subset and could thus be key players in their development.

The results herein also provide a rationale for assessing tumor TRM subsets in both early and late phase studies of novel immunotherapies and cancer vaccines to provide early proof for efficacy as well as potential response biomarkers. The ‘highly functional’ TIM3-expressing TRM subset can be readily isolated from tumor samples using the surface markers identified herein and expanded in vitro to screen and test Tom-targeted adoptive T cell therapies. The highly functional TIM3-expressing TRM subset can be enriched for TAA-specific cells, and specifically expanding this TRM subset will improve the efficacy of adoptive T cell therapies.

It is to be understood that the present disclosure is not limited to particular aspects described, as such may, of course, vary. It is also to be understood that the terminology used herein is for the purpose of describing particular embodiments only, and is not intended to be limiting, since the scope of the present disclosure will be limited only by the appended claims.

A number of embodiments of the disclosure have been described. Nevertheless, it will be understood that various modifications may be made without departing from the spirit and scope of the disclosure. Accordingly, the following examples are intended to illustrate but not limit the scope of disclosure described in the claims.

It is to be inferred without explicit recitation and unless otherwise intended, that when the present technology relates to a polypeptide, protein, polynucleotide or antibody, an equivalent or a biologically equivalent of such is intended within the scope of the present technology.

Throughout this disclosure, various publications, patents and published patent specifications are referenced by an identifying citation. All publications, patent applications, patents, and other references mentioned herein are expressly incorporated by reference in their entirety, to the same extent as if each were incorporated by reference individually. In case of conflict, the present specification, including definitions, will control.

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The disclosure is generally disclosed herein using affirmative language to describe the numerous embodiments and aspects. The disclosure also specifically includes embodiments in which particular subject matter is excluded, in full or in part, such as substances or materials, method steps and conditions, protocols, or procedures. For example, in certain embodiments or aspects of the disclosure, materials and/or method steps are excluded. Thus, even though the disclosure is generally not expressed herein in terms of what the disclosure does not include aspects that are not expressly excluded in the disclosure are nevertheless disclosed herein.

The technology illustratively described herein suitably can be practiced in the absence of any element(s) not specifically disclosed herein. Thus, for example, in each instance herein any of the terms “comprising,” “consisting essentially of,” and “consisting of” can be replaced with either of the other two terms. The terms and expressions which have been employed are used as terms of description and not of limitation and use of such terms and expressions do not exclude any equivalents of the features shown and described or segments thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term “substantially” as used herein refers to a value modifier meaning “at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

Thus, it should be understood that although the present technology has been specifically disclosed by representative embodiments and optional features, modification and variation of the concepts herein disclosed can be resorted to by those skilled in the art, and such modifications and variations are considered within the scope of this technology.

### Methods

### Ethics, Sample Processing and Flow Cytometry

The Southampton and South West Hampshire Research Ethics approved the study, and written informed consent was obtained from all subjects. Newly diagnosed, untreated patients with respiratory malignancies or HNSCC were prospectively recruited once referred. Freshly resected tumor tissue and, where available, matched adjacent non-tumor tissue was obtained from lung cancer patients following surgical resection. Samples were processed as described previously^{70,72}. For sorting of CTLs, cells were first incubated with 4° C. FcR block (Miltenyi Biotec) for 10 min, then stained with a mixture of the following antibodies: anti-CD45-FITC (HI30; BioLegend), anti-CD4-PE (RPA-T4; BD Biosciences), anti-CD3-APC-Cy7 (SK7; BioLegend), anti-CD8A-PerCP-Cy5.5 (cSK1; BD Biosciences), and anti-CD103-APC (Ber-ACT8; Biolegend) for 30 min at 4° C. Live/dead discrimination was by DAPI staining CTLS were sorted based on CD103 expression using a BD FACSAria (BD Biosciences) into ice-cold TRIzol LS reagent (Ambion). HNSCC tumors were macroscopically dissected and slowly frozen in 90% FBS and 10% DMSO (Sigma) for storage until samples could be prepared.

For single-cell transcriptomic, stimulation assays, and phenotypic characterization, tumor and lung samples were first dispersed and cryopreserved in freezing media (50% complete RMPI (Gibco), 40% human decomplemented AB serum, 10% DMSO (both Sigma). Cryopreserved samples were thawed prior to staining with a combination of anti-CD45-AlexaFluor700 (HI30; BioLegend); anti-CD3-APC-Cy7 (SK7; Biolegend); anti-CD8A-PerCP-Cy5.5 (SK1; Biolegend); anti-CD103-Pe-Cy7 (Ber-ACT8; Biolegend); CD19/20 (HIB19/2H7; Biolegend); CD14 (HCD14; Biolegend); CD56 (HCD56; Biolegend) and CD4 (OKT4; Biolegend) for flow cytometric analysis and sorting. Live and dead cells were discriminated using propidium iodide (PI). For 10× single-cell transcriptomic analysis (10× Genomics), 1500 cells each of CD103⁺and CD103⁻ CTLs from tumor and lung samples were sorted and mixed into 50% ice cold PBS, 50% FBS (Sigma) on a BD Aria III or Fusion cell sorter. CTLs for assessments of the bulk transcriptome following stimulation, was collected by sorting 200 cells into 8 μL lysis buffer on an Aria Fusion (BD); for Smart-seq2-based single-cell analysis, CTLs were sorted as above using single cell purity into 4 μL lysis buffer on a BD Aria III as described.

For tumor TRM phenotyping, samples were analyzed on a FACS fusion (BD) following staining with anti-CD45-AlexaFluor700 (HI30; BioLegend); anti-CD3-APC-Cy7 (SK7; Biolegend); anti-CD8A-PerCP-Cy5.5 (SK1; Biolegend); anti-CD103-Pe-Cy7 (Ber-ACT8; Biolegend); CD127-APC (eBioRDR5; eBioscience); anti-CD39-BB515 (TU66; BD); anti-41BB-PE (4B4-1; Biolegend), anti-PD1-BV421 (EH12.1; BD); anti-TIM3-BV605 (F38-2E2; Biolegend). Cells were counter stained with CD19/20 (HIB19/2H7; Biolegend), CD14 (HCD14; Biolegend), CD56 (HCD56; Biolegend) and CD4 (OKT4; Biolegend). Dead cells were discriminated using PI. Phenotypic characterization of lung TRM was completed using the antibodies above with anti-CD49A-PE (SR84; BD) and anti-KLRG1-APC (2F1/KLRG; Biolegend) on a BD LSRII. Data was analyzed in Flowjo 10.4.1, and geometric-mean florescence intensity and population percentage data were exported and visualized in Graphpad Prism (7.0a; Treestar). For tSNE and co-expression analysis of flow cytometry data, each sample was down-sampled to exactly 3,000 randomly selected live and singlet-gated, CD19⁻CD20⁻CD14⁻CD4⁻CD56⁻CD45⁺CD3⁺CD8⁺ CTLs using the gating strategy described above, and 24,000 cells each from the lung and tumor samples were merged to yield 48,000 total cells. A tSNE plot was constructed using 1,000 permutations and default settings in Flowjo 10.4.1, z-score expression was mean centered. Flow cytometry data was exported from FlowJo (using the channel values) and these data were imported into R for co-expression analysis (described below).

### Bulk-RNA Sequencing and TCR-Seq

Total RNA was purified using a miRNAeasy kit (Qiagen) from CD103⁺and CD103⁻ CTLs and was quantified as described previously^{70,72}. For assessment of the stimulated transcriptome, RNA from ˜100 sorted cells was used. Total RNA was amplified according to the Smart-seq2 protocol. cDNA was purified using AMPure XP beads (0.9:1 ratio, Beckman Coulter). From this step, 1 ng of cDNA was used to prepare a standard Nextera XT sequencing library (Nextera XT DNA sample preparation and index kits, Illumina). Samples were sequenced using an Illumina HiSeq2500 to obtain 50-bp single-end reads. For quality control, steps were included to determine total RNA quality and quantity, the optimal number of PCR pre-amplification cycles, and cDNA fragment size. Samples that failed quality control or had a low number of starting cells were eliminated from further sequencing and analysis. TCR-seq was performed as previously described³¹, using Tru-seq single indexes (Illumina). Sequencing data was mapped and analyzed using MIGEC software with default settings, followed by V(D)J tools with default settings. Mapping QC matrices are included in (Table 6).

### 10× Single-Cell RNA Sequencing

Samples were processed using 10×v2 chemistry as per manufacturer's recommendations; 11 and 12 cycles were used for cDNA amplification and library preparation respectively. Barcoded RNA was collected and processed following manufacturer recommendations, as described previously. Libraries were sequenced on a HiSeq4000 (Illumina) to obtain 100- and 32-bp paired-end reads using the following read length: read 1, 26 cycles; read 2, 98 cycles; and i7 index, 8 cycles. Samples were pooled together DNA samples from whole blood were extracted using a High salt method and were quantified using the Qubit 2.0 (Thermo). Genotyping was completed through the Infinium Multi-Ethnic Global-8 Kit (Illumina), following the manufacturer's instructions. Raw data from the genotyping analysis was exported using Genotyping module and Plug-in PLINK Input Report Plug-in (v2.1.4) from GenomeStudio v2.0.4 (Illumina). The data quality was assessed using the snpQC package with R and low-quality SNPs were detected: SNPs failing in more than 5% of the samples and SNPs with Illumina's GC scores less than 0.2 in more than 10% of the samples were flagged. Subjects' sex was matched with the genotype data and flagged SNPs were removed for downstream analysis using PLINK (v1.90b3w). Genetic multiplexing of barcoded single-cell RNA-seq was completed using Demuxlet and matched with the Seurat output. Cells with ambiguous or doublet identification were removed from analysis of cluster and/or donor proportions.

### Bulk-RNA-Seq Analysis

Bulk RNA-Seq data were mapped against the hg19 reference using TopHat (v2.0.9 (--library-type fr-unstranded --no-coverage-search) and htseq-count -m union -s no -t exon gene_name (part of the HTSeq framework, version 0.7.1)). Trimmomatic (0.36) was used to remove adapters. Values throughout are displayed as log₂ TPM (transcripts per million); a value of 1 was added prior to log transformation. To identify genes expressed differentially by various cell types, negative binomial tests for paired comparisons by employing the Bioconductor package DESeq2 (1.14.1) were performed, disabling the default options for independent filtering and Cooks cutoff. The Applicants considered genes to be expressed differentially by any comparison when the DESeq2 analysis resulted in a Benjamini-Hochberg-adjusted P value of <0.05 and a fold change of at least 2. Union gene signatures were calculated using the online tool jVenn, of which genes must have common directionality. GSEA, correlations, and heatmaps were generated as previously described^{31,72} For the preservation of complementary signatures, data from Cheuk, et al 2017 was downloaded from code GSE83637 and differential expressed was completed as above, for the murine composite signature, orthologues between human and murine signatures were compared using Biomart. Reactome pathways were generated using the online tool for tumor T_{RM}-specific genes, a pathway was considered significantly different if the FDR (q) values was <0.05 (Table 5). Visualizations were generated in ggplot2 using custom scripts, while expression values were calculated using Graphpad Prism? (7.0a). For tSNE analysis, the data frame was filtered to genes with >1 TPM expression in at least one condition and visualizations created using the top 2000 most variable genes, as calculated in DESeq2 (1.18.1); this allowed for unbiased visualization of the Log₂ (TPM+1) data, using package Rtsne (0.13). Co-expression networks were generated in gplots (3.0.1) using the heatmap2 function, while weighted correlation analysis was completed using WGCNA (1.61) from the Log₂ (TPM+1) data matrix and the function exportNetworkToCytoscape at Beta=5, weighted=true, threshold=0.05. Networks were generated in Gephi (0.92) using Fruchterman Reingold and Noverlap functions. The size and color were scaled according to the Average Degree as calculated in Gephi, while the edge width was scaled according to the WGCNA edge weight value. The statistical analysis of the overlap between gene sets was calculated in R (v3.5.0) using the fisher.test function (Stats—v3.5.0) using the number of total quantified genes used for DESeq2, as the total value, with alternative=“greater”.

Single-cell RNA-Seq analysis Raw 10× data was processed as previously described³¹, merging multiple sequencing runs using cellranger count function in cell ranger, then merging multiple cell types with cell ranger aggr. The merged data was transferred to the R statistical environment for analysis using the package Seurat (v2.2.1). Only cells expressing more than 200 genes and genes expressed in at least 3 cells were included in the analysis. The data was then log-normalized and scaled per cell and variable genes were detected. Transcriptomic data from each cell was then further normalized by the number of UMI-detected and mitochondrial genes. A principal component analysis was then run on variable genes, and the first 8 principal components (PCs) were selected for further analyses based on the standard deviation of PCs, as determined by an elbow plot in Seurat. Cells were clustered using the FindClusters function in Seurat with default settings, resolution=0.6 and 8 PCs. Differential expression between clusters was determined by converting the data to CPM and analyzing cluster specific differences using MAST (q<0.01). A gene was considered significantly different, only if the gene was commonly positively enriched in every comparison for a singular cluster³¹. Further visualizations of exported normalized data were generated using the Seurat package and custom R scripts. Cell-state hierarchy maps were generated using Monocle version 2.6.1³⁰ and default settings, including the most variable genes identified in Seurat for consistency. Average expression across a cell cluster was calculated using the AverageExpression function, and downsampling was achieved using the SubsetData function (both in Seurat). Distance between clusters was calculated by calculating a particular cells location in PCA space (Principle component 1:3) using the function GetCellembeddings (in Seurat), the values for each cell were then scaled per column (Scale function, core R) where described, and finally a distance matrix was calculated (dist function, core R, method=euclidean). This matrix was filtered to the cells assigned to cluster 1, and the mean distance of each cell in cluster 1 to all cells in each of the remaining TRM clusters (2,3,4,5) was calculated. The clustering analysis was completed using the hclust function in R (stats, R v3.5.0) with average linkage and generated from the spearman correlation analysis of each cell's location in PCA space (as above). SAVER co-expression analysis was completed on the raw-UMI counts of the TRM cells (clusters 1-5) and the non-TRM cells (remaining cells) using the function saver (v1.1.1) with pred.genes.only=TRUE, estimates.only=FALSE on transcripts assigned as uniquely enriched in cluster 2, removing genes not expressed in any cells in the non-TRM compartment. Correlation values were isolated using the cor.genes function in SAVER and co-expression plots generated as described above. Smart-seq2 single cell analysis was completed as previously described using TraCer and custom scripts to identify αβ chains and to remove cells with low QC values as previously described. Here, cells with fewer than 200,000 reads and lesser than 30% of sequenced bases assigned to mRNA were removed. Samples were mapped as described for the bulk population analysis, and the data was log transformed and displayed as normalized TPM counts; a value of 1 was added to low or zero values prior to log transformation. Visualizations were completed in ggplot2, Prism v7 (as above) and custom scripts in TraCer. A cell was considered expanded when both the most highly expressed α and β TCR chain sequences matched other cells with the same criteria. Cells were considered not expanded when neither a or 13 TCR chain sequences matched those of any other cells. A cell was considered TIM3⁺when the expression of HAVCR2 was greater than 10 TPM, while a cell was considered cycling if expression of cell cycle genes TOP2A and/or MKI67 was greater than 10 TPM. Differential expression profiling was completed using MAST (q<0.05) as previously described³¹.

Matched flow cytometry data was analyzed using FlowJo v10.4.1, values and gates were exported into ggplot and “in-silico gates” were applied using custom scripts in R. Given ˜85% of the CD103+ cells were TIM-3+ from the flow cytometry data, cells were broadly classified into TRM or non-TRM based on an individual cell's protein expression (FACS gating). Where there was no available cell-specific associated protein data, CD3+ T cells were classified based on the lack of expression of CD4 and FOXP3, to remove CD4+ cells. Next, the single cell transcriptomes were stratified into TRM or non-TRM cells when expression of TRM associated genes, ITGAE (CD103), RBPJ and/or ZNF683 (HOBIT) were greater than 10 TRM counts. Differential gene expression analysis was completed as above.

### Multiplex Immunohistochemistry

Patients included in this cohort had a known diagnosis of lung cancer. 23 patients were selected in total, categorizing the donors using criteria previously reported9. A multiplexed IHC method was utilized for repeated staining of a single paraffin-embedded tissue slide. Deparaffinisation, rehydration, antigen retrieval and IHC staining was carried out using a Dako PT Link Autostainer. Antigen retrieval was performed using the EnVision FLEX Target Retrieval Solution, High pH (Agilent Dako) for all antibodies. The slide was first stained with a standard primary antibody followed by an appropriate biotin-linked secondary antibody and horseradish peroxidase (HRP)-conjugated streptavidin to amplify the signal. Peroxidase labelled compounds were revealed using 3-amino-9-ethylcarbazole (AEC), an aqueous substrate that results in red staining, or DAB that results in brown staining, and counter stained using hematoxylin (blue).

The slides were stained initially with Cytokeratin (pre-diluted, Clone AE1/AE3; Agilent Dako) then sequentially with anti-CD8a (pre-diluted Kit IR62361-2; clone C8/144B; Agilent Dako), anti-CD103 (1:500; EPR4166(2); abcam) and anti-TIM-3 (1:50; D5D5R; Cell Signaling Technology). The slides were scanned at high resolution using a Zeiss Axio Scan.Z1 with a 20× air immersion objective. Between each staining iteration, antigen retrieval was performed along with removal of the labile AEC staining and denaturation of the preceding antibodies using a set of organic solvent based de-staining buffers; 50% ethanol for 2 minutes; 100% ethanol for 2 minutes; 100% xylene for 2 minutes; 100% ethanol for 2 minutes; 50% ethanol for 2 minutes. This process did not affect DAB staining. The process was repeated for each of the antibodies.

Bright field images were separated into color channels in imaging processing software ImageJ FIJI81 (ImageJ Windows 64-bit final version). For the TIL^{high}TRM^{high} and TIL^{low}TRM^{low} tumors the number of CD8+CD103+TIM3+ cells were quantified manually. Two samples with ≤3 CD8+CD103+ CTLs quantified were removed, to prevent calculating percentages of single events, resulting in a final number of 21 samples. These images were processed and combined to create pseudo-color multiplexed images. The raw counts for each protein, individually and together are presented in Table 7, as the number of cells per 0.15 mm2.

### OMNI-ATAC-Seq

CTLs were FACS sorted from cryopreserved lung cancer samples as described above, using the following antibody cocktail: anti-CD45-AlexaFluor700 (HI30; BioLegend); anti-CD3-APC-Cy7 (SK7; BioLegend); anti-CD8A-PerCP-Cy5.5 (SK1; BioLegend); anti-CD103-Pe-Cy7 (Ber-ACT8; BioLegend); anti-CD127-APC (eBioRDR5; ThermoFisher); anti-TIM-3-BV605 (F38-2E2; BioLegend). Cells were counter stained with anti-CD19/20-PEDazzle (HIB19/2H7; BioLegend); anti-CD14-PE-Dazzle (HCD14; BioLegend); and anti-CD4-BV510 (OKT4; BioLegend). Dead cells were discriminated using PI. Samples were sorted into low retention 1.5 ml eppendorfs containing 250 μL FBS and 250 μL PBS. Three to six donors were pooled together to guarantee sufficient cell numbers. For each pool of cells, two or three technical replicates of 15,000-25,000 CTLs were generated for each library.

OMNI-ATAC-seq was performed as described in Corces, et al., with minor modifications. Isolated nuclei were incubated with tagmentation mix (2×TD buffer, 2.5 μL transposase enzyme from Nextera kit, Illuminia) at 37° C. for 30 minutes in a thermomixer, shaking at 1000 RPM. Following tagmentation, the product was eluted in 0.1× Tris-EDTA buffer using DNA Clean and Concentrator-5 kit (Zymo). The Purified product was preamplified for 5 cycles using Kappa 2× enzyme along with Nextera indexes (Illumina) and based on qPCR amplification, an additional 7 cycles of amplification was performed for 20,000 cells. The PCR amplified product was purified using DNA Clean and Concentrator-5 kit (Zymo), and size selection was done using AMPure XP beads (Beckman Coulter). Finally, concentration and quality of libraries were determined by picogreen and bioAnalyzer assays. Equimolar libraries were sequenced as above, or on a NovaSeq 6000 for sequencing.

Next, technical replicates were randomly down sampled to between 25,000,000 to 40,000,000 total reads and merged using Bash scripts, resulting in two TIM-3+IL-7R-TRM pools and two non-TRM pools. These reads were mapped to hg19 with bowtie2 (v2.3.3.1). Chromosomes 1-22, and X were retained, chrY, chrM, and other arbitrary chromosome information based reads were removed. Samtools (v1.9) was used to get the uniquely mappable reads, only reads MAPQ≥30 were considered. Duplicate reads are removed by “MarkDuplicates” utility of Picard tool (v 2.18.14). Before peak calling, tag align files were created, by shifting forward strands by 4 bases, and reverse strands by 5 bases (TN5 shift). Peaks were identified with MACS2 (v 2.1.1.20160309) using the function. -f BED -g ‘hs’-q 0.01 --nomodel --nolambda --keep-dup all --shift -100 --extsize 200. BamCoverage (v2.4.2) was used for converting bam files into bigwig, and further UCSC track generation (same normalization across all ATACseq and RNAseq samples), as per the following example: bamCoverage -b TIL_103 pos.bam -o TIL_103 pos_NormCov.bw -of bigwig -bs 10 --normalizeTo1x 2864785220 --normalizeUsingRPKM -e 200. The R package DiffBind (v2.2.12) was used to highlight differentially accessible peaks (based on DEseq2). R packages of org.Hs.eg.db (v3.4.0 and TxDb.Hsapiens.UCSC.hg19.knownGene (v3.2.2) were used to annotate peaks. Following differential expression peaks were filtered to those within 5 kb of a transcription start site to focus directly on promoter accessibility. The correlation plot (spearman) was completed as described above, using all identified peaks. The plot was clustered according to complete linkage.

### Statistical Analysis

The significance of differences among matched samples were determined by Wilcoxon matched-pairs signed rank test unless otherwise stated. Statistical analyses were performed using Graphpad Prism? (7.0a). Spearman correlation coefficient (r value) was used to access significance of correlations between the levels of any two components of interest.

### Data Availability

Sequencing data was deposited into the Gene Expression Omnibus.

Immunotherapy is rapidly becoming a mainstream treatment of solid cancers^{[51,52]}; nonetheless, less than 30% of patients benefit from this approach^{[53]}. Thus, there is an urgent need to develop novel immunotherapeutic agents for the patients who do not respond to currently available immunotherapies. Applicants' goal is to identify such novel targets by systematically investigating the molecular mechanisms that drive the development and function of a novel class of cytotoxic T lymphocytes (CTLs) in the tumor immune microenvironment (TIME)—tissue-resident memory cells (T_{RM}), which Applicants have recently shown to be key players in driving effective anti-tumor immune responses in lung cancer^{[54]}. This breakthrough finding (Nature Immunology 2017) was possible because of the ongoing collaboration between the Applicants.

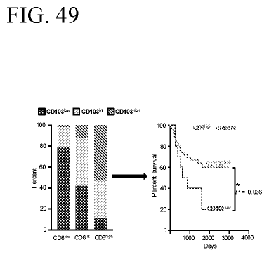

Tissue-resident memory (Tam) CTLs in cancer: Applicants were the first to conclusively show that higher density of T_{RM} cells in tumor tissue (defined here as ‘immune hot’ tumors) predicted better survival outcomes in human cancers, and that this effect was independent of that conferred by the density of the global CD8⁺ T cell population in tumors^{[101]} (FIG. 49). To understand the molecular features that drive efficient T_{RM} immune responses in the TIME, Applicants performed single-cell and bulk RNA-Seq analysis of purified populations of TRM and non-T_{RM} cells present in tumor and normal lung tissue from lung cancer patients. The key results were: (i) The identification of a novel TIM-3 expressing TRM subset present exclusively in tumors. This subset also expressed high levels of PD-1. Surprisingly, however, they proliferated in the tumor milieu, released effector cytokines when stimulated ex-vivo and exhibited a transcriptional program indicative of superior effector, survival and tissue residency properties (FIG. 2-FIG. 4). This ‘highly functional’ PD-1+TIM-3+T_{RM} subset was validated by functional assays ex-vivo and reflected in the chromatin accessibility profile of this subset. This TIM-3+IL-7R-TRM subset was enriched in responders to PD-1 inhibitors and in tumors with a greater magnitude of CTL responses. These data highlights that not all CTLs expressing PD-1+ are dysfunctional, in particular, TRM cells with the highest PD-1 expression are enriched for features of superior functionality. (ii) Definition of a core set of genes that were enriched in the ‘highly functional’ PD-1⁺TIM-3⁺ T_{RM} subset in tumors, which included a number of novel genes (e.g., AMICA, SIPRG, KIR2DL4) whose expression was highly correlated with known tissue residency (T_{RM}) genes. Any of these genes are likely to be critically important for the development, trafficking or function of tumor-infiltrating TRM cells. (iii) M1^{hot} myeloid cells in the TIME were associated with robust TRM anti-tumor responses. This finding was revealed by Applicants' novel integrated weighted gene correlation network analysis (iWGCNA) analysis of matched CTLs and myeloid cells.

Applicants hypothesize, without being limited to a particular theory: (i) ‘Highly functional’ PD-1⁺TIM-3⁺ T_{RM} subset are increased in numbers and qualitatively superior in patients with ‘immune hot’ tumors and in ‘responders’ to anti-PD1 therapy. (ii) Expansion of this TRM subset in ‘immune hot’ tumors is positively correlated with expansion of myeloid subsets (M1^{hot}) that promote anti-tumor immunity. (iii) ‘Candidate molecules’ (AMICA, SIRPG, CD38 etc.,) whose expression is enriched in ‘highly functional’ TRM cells are promising immunotherapy targets to boost anti-tumor TRM responses.

### Results

The identification of molecular players and pathways that lead to the generation of effective anti-tumor TRM immune responses will inform the discovery of new drug targets for treating cancer. Current knowledge of these players is vastly incomplete, as investigative studies are mainly focused on genes and molecules identified based on a priori concepts in immunology and cell biology and have thus far neglected the study of tumor-infiltrating TRM cells. Applicants' team performed the first and largest unbiased survey of bulk and single-cell transcriptomes from purified TRM CTLs isolated from tumors of patients with cancer.

T_{RM} CTL responses have also recently been shown by Applicants⁹ and others¹⁰ to be associated with better survival in patients with solid tumors. The molecular features of TRM cells' responses have been characterized in infection models, and involve rapid clonal expansion and upregulation of molecules aiding recruitment and activation of additional immune cells alongside the conventional effector functions of CTLs¹¹. To date, the properties of TRM cells found in the background lung, compared to those in the tumor are not fully elucidated. Furthermore, the properties of these cell subsets in the context of immunotherapy are still poorly understood. To address this question, Applicants compared the transcriptome of TRM and non-T_{RM} CTLs present in tumor and normal lung tissue samples from treatment naïve patients with lung cancer. Furthermore, Applicants investigated the same tissue resident populations in head and neck squamous cell carcinoma and during immunotherapy regimes. Key results are summarized below:

### Shared Features of TRM Cells in Human Lungs and Tumor.

Applicants compared the transcriptome of CTLs isolated from lung tumor and adjacent uninvolved lung tissue samples obtained from patients (n=30) with treatment-naïve lung cancer, sorted according to CD103 expression to separate TRM from non-T_{RM} cells. Lung CD103⁺ and CD103⁻ CTLs clustered separately and showed differential expression of nearly 700 transcripts including several previously linked to TRM phenotypes (FIG. 2). These data confirm that CD103⁺ CTLs in human lungs and tumors are highly enriched for TRM cells; hereafter Applicants refer to CD103⁺ CTLs as TRM cells and CD103⁻ CTLs as non-T_{RM} cells. Applicants next asked if TRM cells in lung tumors share tissue residency features with TRM cells in adjacent normal lung tissue. Nearly one-third (89/306) of the TRM properties, i.e., transcripts differentially expressed between CD103⁺and CD103⁻ CTLs in tumors that were shared with those of normal lung TRM cells (FIG. 2C, venn diagram). Weighted gene co-expression network analysis (WGCNA) of the 89 ‘shared tissue residency’ transcripts revealed a number of novel genes whose expression was highly correlated with known tissue residency genes (S1PR1, S1PR5, ITGA1, HOBIT, RBPJ^{12,13}), suggesting that their products may also play important roles in the development, trafficking or function of T_{RM} cells (FIG. 2E). Notable examples encoding products likely to be involved in T_{RM} functionality, migration or retention include SRGAP3¹⁷, AMICA1¹⁸, CAPG¹⁹, ADAM19²⁰, and NUAK2²¹ (FIG. 2E).

### PD-1 Expression is a Feature of Tumor and Lung TRM Cells.

Another important ‘shared tissue residency’ transcript was PDCD1, encoding PD-1 (FIG. 2E). Although PD-1 expression is considered typical of exhausted T cells as well as activated cells³, recent reports have suggested that high PD-1 expression is a tissue residency feature of brain TRM cells independent of antigen stimulation^{22,23}, and of murine T_{RM} cells from multiple organ systems¹⁴. In support of the conclusion that high expression of PD-1 reflects tissue residency, rather than exhaustion, Applicants found that when T_{RM} and non-T_{RM} cells isolated from both lung and tumor tissue were stimulated ex vivo, they showed robust up-regulation of TCR-activation-induced genes and cytokines (TNF, IFNG) (data not shown). In addition to PDCD1, ‘shared tissue-residency’ transcripts included several (SPRY1²⁴, TMIGD2²⁵, CLNK²⁶) that encode products reported to play a regulatory role in other immune cell types (FIG. 2E). Applicants speculate that the expression of these inhibitory molecules may restrain the functional activity of tumor TRM cells and may represent targets for future immunotherapies.

Tumor TRM Cells were Clonally Expanded, Proliferate and Express Markers of Enhanced Function.

To identify features unique to tumor TRM cells, Applicants compared the transcriptome of TRM cells and non-T_{RM} cells from both normal lung and tumors and detected 93 differentially expressed transcripts (FIG. 3A) specifically in this subset, hence termed ‘tumor T_{RM}-enriched’ transcripts. Reactome pathway analysis of these ‘tumor T_{RM}-enriched’ transcripts showed significant enrichment for transcripts encoding components of the canonical cell cycle, mitosis and DNA replication machinery (FIG. 3B). The tumor TRM subset thus appears to be highly enriched for proliferating CTLs, presumably responding to tumor-associated antigens (TAA), despite PD-1 expression. Unique molecular identifier (UMI)-based T cell receptor (TCR) sequencing assays revealed that TRM cells in tumors expressed a significantly more restricted TCR repertoire than non-TRM cells in tumors. Furthermore, the tumor TRM population contained a higher mean percentage of expanded clonotypes (73% versus. 52%, in tumor TRM versus. non-T_{RM} populations, data not shown).

‘Tumor T_{RM}-enriched’ transcripts that were highly correlated with cell cycle genes may encode products with important functions, as they are likely to reflect the molecular features of TRM cells that are actively expanding in response to TAA. HAVCR2, encoding the co-inhibitory checkpoint molecule TIM-3, was most correlated and connected with cell cycle genes (FIG. 3E). Thus, TIM-3 expression may be a feature of lung tumor TRM cells that is not linked to exhaustion, but rather reflects a state of ‘high functionality, as the other transcripts that correlated with expression of TIM-3 and cell cycle genes encode molecules that likely confer superior functionality, such as CD39 (encoded by ENTPD1)³⁰, CXCL13³¹, CCL3³², TNFSF4³³ (OX-40 ligand), as well as a marker of antigen-specific engagement (4-1BB)³⁴ (FIG. 3E). Robust expression of this set of molecules was not observed in either human lung TRM cells or in the mouse TRM signatures^{13,14,35}, indicating that the tumor TRM population contains novel cell subsets.

### Single-Cell Transcriptomic Analysis Reveals Previously Uncharacterized TRM Subsets.

To determine whether ‘tumor T_{RM}-enriched’ transcripts are expressed in all or only a subset of the tumor TRM population, Applicants performed low resolution (10× platform) single-cell RNA-seq assays in CD103⁺and CD103⁻ CTLs isolated from tumor and matched adjacent normal lung tissue from 12 patients with early-stage lung cancer. Analysis of the ˜12,000 single-cell transcriptomes revealed 5 clusters of TRM cells and 4 clusters of non-T_{RM} cells (FIG. 4A, FIG. 4B). Among the 5 TRM clusters, clusters 1-3 contained a greater proportion of the tumor TRM population while clusters 4 and 5 contained more lung TRM cells (FIG. 4C). Most strikingly, clusters 1-3 contained very few lung TRM cells (FIG. 4C). Applicants infer that the ‘tumor T_{RM}-enriched’ transcripts detected in Applicants' analysis of bulk populations were likely to be contributed by cells in these subsets. In agreement with that conclusion, cells in cluster 1 expressed high levels of the 25 cell cycle-related ‘tumor T_{RM}-enriched’ transcripts³⁶, indicating that the enrichment of cell cycle transcripts in the bulk tumor TRM population was contributed by this relatively small subset. Because these cells are actively proliferating, they likely represent TAA-specific cells. The majority of cells in this cycling cluster were from the tumor TRM population. These cells, as well as those in the larger cluster 2, were highly enriched for other prominent ‘tumor Tom-enriched’ transcripts like HAVCR2 (TIM-3), including those encoding products that could confer superior functionality (e.g., CD39⁹, CXCL13³¹, CCL3³²), consistent with recent reports^{28,29}. This shared expression pattern suggests that the cycling cluster (cluster 1) may simply represent cells in cluster 2 that are entering the cell cycle. Confirming this idea, cell-state hierarchy maps of all TRM cells, constructed using Monocle2³⁷, revealed that cells in cluster 2 were most similar to the cycling TRM cells (cluster 1, data not shown). Overall, Applicants' single-cell transcriptome uncovered additional phenotypically distinct subsets of tumor TRM cells that have not previously been described and are likely to play an important role in anti-tumor immune responses.

TIM-3⁺IL7R⁻ TRM Subset has a Transcriptional Program Indicative of Superior Functional Properties.

Because of their close relationship with cycling TRM cells, Applicants focused Applicants' analysis on the TRM cells in cluster 2. The 91 transcripts enriched in cluster 2 compared to the other TRM clusters included several which encoded products linked to cytotoxic activity such as PRF1, GZMB, GZMA, CTSW³⁸, and CRTAM³⁸, as well as transcripts encoding effector cytokines and chemokines such as IFNG, CCL3, CXCL13, IL17A and IL26 (FIG. 5C and data not shown). Cluster 2 also expressed high levels of transcripts encoding transcription factors known to promote the survival of memory or effector CTLs (ID2⁴⁵, STAT3⁴⁶, ZEB2⁴⁷) or those that are involved in establishing and maintaining tissue residency (RBPJ, a key player in Notch signaling¹³, and BLIMP1³⁵, encoded by PRDM1). TRM cells in cluster 2 also strongly expressed ENTPD1, which encodes CD39, an ectonucleotidase that cleaves ATP, which may protect this TRM subset from ATP-induced cell death in the ATP-rich tumor microenvironment³⁰ and has recently been shown to be enriched for tumor neo-antigen specific CTLs^{49,50}. This expression pattern likely confers highly effective anti-tumor immune function; in combination with earlier results, Applicants conclude that this ‘highly functional TIM-3⁺IL7R⁻^{^{TRM}} subset’ likely represents TAA-specific cells that were enriched for transcripts linked to cytotoxicity.

Intriguingly, TRM cells in cluster 2 (TIM-3⁺IL7R⁻ subset) expressed the highest levels of PDCD1 transcripts and were enriched for transcripts encoding other molecules linked to inhibitory functions such as TIM-3, TIGIT⁵¹, and CTLA4⁵²⁻⁵⁴. Nonetheless, these TRM cells exhibited a transcriptional program suggestive of superior effector properties and cell proliferation expressed high transcript levels for cytotoxicity molecules (Perforin, Granzyme A and Granzyme B) and several co-stimulatory molecules such as 4-1BB, ICOS and GITR (TNFRSF18) (FIG. 5C and data not shown).³ More specifically, PDCD1-expressing TRM cells in cluster 2 expressed relatively higher levels of IFNG, CCL3, and CXCL13 transcripts compared with cells not expressing PDCD1 in that cluster and other tumor-infiltrating TRM and non-T_{RM} cells (FIG. 5D. Overall, these findings agree with the bulk RNA-seq analysis, indicating that expression of inhibitory molecules, like PD-1, does not reflect exhaustion. Instead, it may prevent TCR-activation-induced cell death to sustain robust anti-tumor CTL responses.

### PD-1- and TIM-3-Expressing Tumor-Infiltrating TRM Cells are not Exhausted.

To further support the case that PDCD1-expressing TRM cells in cluster 2 (TIM-3⁺IL7R⁻ ‘highly functional’ TRM cells) are not exhausted, but instead highly functional, Applicants performed single-cell RNA-seq in tumor-infiltrating TRM and non-T_{RM} cells, using the more sensitive Smart-seq2 assay for paired transcriptomic and TCR clonotype analysis³⁸. As expected, clonally expanded tumor-infiltrating TRM cells, which are likely to be reactive to TAA, were significantly enriched for genes specific to ‘highly functional’ TIM-3⁺IL7R⁻ TRM cells. Among tumor-infiltrating CTLs, a greater proportion of TIM-3-expressing TRM cells were clonally expanded compared with other TRM and non-T_{RM} cells (FIG. 6A, FIG. 6B). Furthermore, TIM-3-expressing TRM cells were significantly enriched for key effector cytokines and cytotoxicity transcripts, despite expressing significantly higher levels of PDCD1 (data not shown). The higher sensitivity of the SMART-seq2 assay compared to the 10× genomics platform also allowed better co-expression analysis³⁸. Specifically, IFNG and PDCD1 expression levels were better correlated in TIM-3-expressing TRM cells compared with non-T_{RM} cells (FIG. 6D), and the proportion of cells strongly co-expressing these transcripts was notably higher (30% versus. 1%). Overall, these results strongly support that PD-1 and TIM-3 expressing tumor-infiltrating TRM cells were not exhausted, but instead were enriched for transcripts (IFNG, PRF1, GZMA) encoding for molecules linked to effector functions are “highly functional.”

### Functional and Protein Validations.

In keeping with Applicants' transcriptomic assays, when stimulated ex-vivo, tumor-infiltrating TRM cells that co-expressed PD-1 (stained before stimulation) had significantly higher percentage of cells expressing effector cytokines, when compared to the non-T_{RM} CTLs that co-expressed PD-1 (FIG. 50). Analysis directly ex-vivo demonstrated there was also greater expression of cytotoxic-associated proteins, granzyme A and granzyme B, in the PD-1⁺ TRM cells when compared to the PD-1⁺non-T_{RM} CTLs in the tumor (FIG. 50). These data verify that PD-1 expression in the TRM subset of tumor-infiltrating CTLs does not reflect dysfunctional properties.

### Tumor Specificity the Highly Functional Tumor TRM Cells.

TIM-3-expressing CTLs were also detected among tumor-infiltrating TRM cells isolated from both treatment naïve lung cancer and head and neck squamous cell carcinoma (HNSCC) samples, but not among non-T_{RM} cells in these treatment naïve tumors or TRM cells in lung. These finding confirmed, at the protein level, the specificity of the TIM-3⁺IL-7R⁻ TRM subset to tumors from two cancer types studied.

Applicants' bulk and single-cell transcriptomic analysis of purified population of TRM cells showed that the molecular program of tumor-infiltrating TRM cells is substantially distinct from that observed in the human background lung tissue or in murine models. The most striking discovery was the identification of a ‘highly functional’ TIM-3-expressing TRM subset present exclusively in tumors. This subset expressed high levels of PD-1 and other molecules previously thought to reflect exhaustion. Surprisingly however, they proliferated in the tumor milieu, were capable of robust up-regulation of TCR-activation-induced genes and protein expression of cytokines when stimulated ex vivo and exhibited a transcriptional program indicative of superior effector, survival and tissue residency properties. T_{RM} subsets and their molecular properties that associate with response to anti-PD1 therapy.

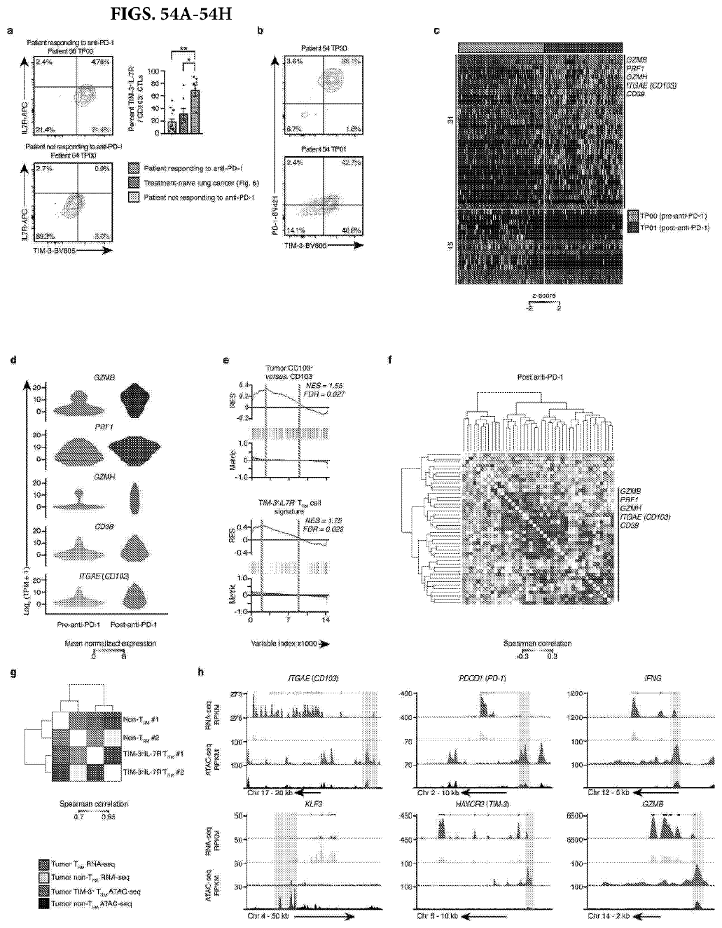

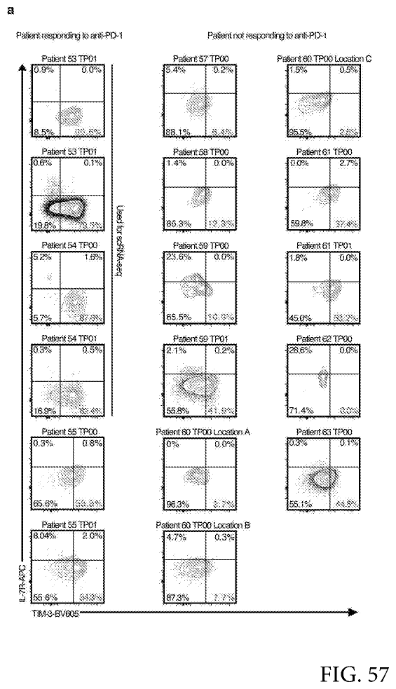

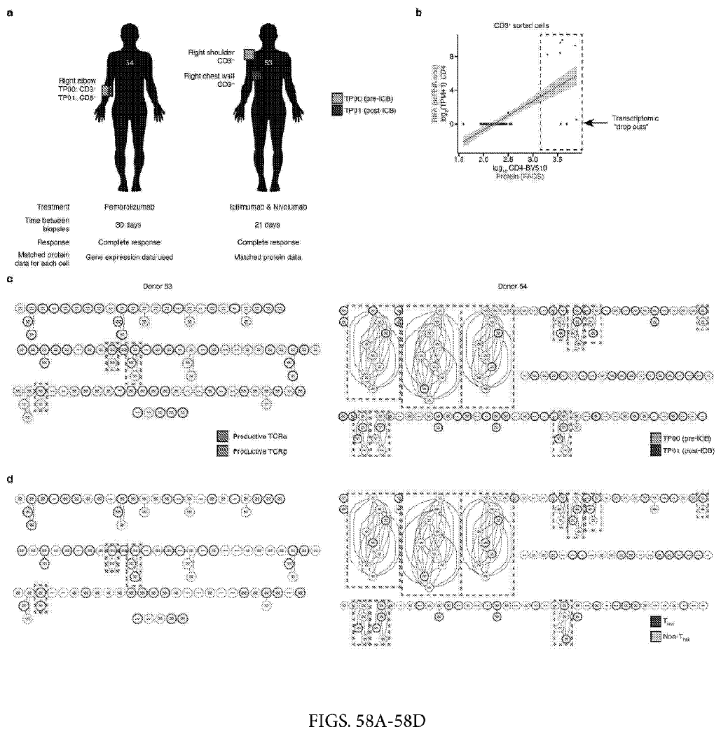

Analysis of CTLs from anti-PD-1 responders Applicants analysed tumor-infiltrating T cells from 19 biopsies (FIG. 54) with known divergent responses to anti-PD-1 therapy. Flow cytometry analysis of tumor TRM cells isolated from responding patients pre-, during-, and post-treatment samples showed an increased proportion of TIM-3⁺IL-7R⁻^{^{TRM}} cells when compared to the tumor TRM cells from Applicants' cohort of treatment naïve lung cancer patients and those not responding to anti-PD-1 (˜70% versus. ˜24% and ˜9%, respectively; (FIG. 54, FIG. 56B, FIG. 57). Pre-anti-PD-1 therapy that was diminished post-treatment is likely reflective of the clinical antibody blocking flow cytometric analysis (FIG. 54B) Since this population also expressed high levels of PD-1 (FIG. 6F, FIG. 54B) Applicants show that these TRM cells may be the key responder cells to anti-PD-1 therapy. To comprehensively evaluate the molecular features and clonality of the CTLs (FIG. 58A, FIG. 58B) responding to anti-PD-1 therapy, Applicants performed paired single-cell transcriptomic and TCR analysis of CTLs isolated from biopsies both pre- and post-therapy from two donors. Differential expression analysis of all CD8+ tumor-infiltrating CTLs revealed a significant enrichment of markers linked to cytotoxic function (PRF1, GZMB and GZMH) and activation (CD38) in post-treatment compared to pre-treatment samples (FIG. 54C, FIG. 54D). Furthermore, Applicants found sharing of TCR clonotypes (FIG. 58C, FIG. 58D) between CTLs from post and pre-treatment samples (not shown), which suggested that tumor-infiltrating CTLs with the same specificity displayed enhanced cytotoxic properties following anti-PD-1 treatment. Notably, Applicants found increased expression of ITGAE, a marker of TRM cells, in CTLs from post-treatment samples (FIG. 54C, FIG. 54D). GSEA analysis also showed that tumor-infiltrating T cells from post-treatment samples were enriched for TRM features as well as those linked to TIM3⁺IL7R⁻ TRM subset (FIG. 54E, Table 4). Unbiased co-expression analysis of transcripts from post-treatment CTLs demonstrated that that transcripts linked to cytotoxicity (GZMH) and activation (CD38) clustered together with the TRM marker gene (ITGAE; FIG. 54F). Together, these results indicated that anti-PD-1 treatment enhanced the cytotoxic properties of tumor-infiltrating CTLs and that TRM cells largely contributed to this feature.

To provide a further line of evidence for the functional potential of TIM-3+IL-7R-TRM cells and to further characterize their epigenetic profile, Applicants performed OMNI-ATAC-seq on purified populations of tumor-infiltrating TIM3+IL7R-TRM and non-TRM subsets pooled from lung cancer patients (n=9, FIG. 7, FIG. 13). These subsets clustered separately, highlighting the distinct chromatin accessibility profiles of these populations (FIG. 54G). In keeping with transcriptomic analyses (FIG. 2D), Applicants identified greater chromatin accessibility within 5 kb of the transcriptional start site of the CD103 (ITGAE) and KLF3 loci, in the TRM and non-TRM compartment, respectively. Furthermore, consistent with single-cell transcriptional data, the TIM3+IL7R-TRM cells when compared to non-TRM cells showed increased chromatin accessibility of genes encoding effector molecules such as granzyme B and IFN-γ, despite showing increased accessibility at the PDCD1 (PD-1) and TIM-3 (HAVCR2) loci (FIG. 54H). Taken together, these epigenetic and transcriptomic data, combined with protein validation highlighted the potential functionality of the TIM-3+IL-7R-TRM cells, which positively correlated with expression of PD-1 specifically in this subset.

Based on the above findings, Applicants hypothesize, without being limited to a particular theory, that the highly functional ‘PD-1⁺TIM-3⁺ TRM subset is one of the key responder cell types to anti-PD1 therapy.

Functional Analysis of Novel Molecules Linked to TRM CTL Development and/or Function.

New molecules linked to TRM immune response: In Applicants' transcriptomic study of total CD8⁺ TILs, transcripts for molecules that have been shown to be effective immunotherapy targets such as PD-1 and TIM-3 were among the most enriched in tumors with CD8^{high} and CD103^{high} TIL status, which were both independently linked to better anti-tumor immunity and survival outcome. Therefore, Applicants reasoned that other molecules in the list of genes upregulated in tumors with CD8^{high} and CD103^{high} TIL status might also play an important functional role in modulating the magnitude and specificity of anti-tumor immune responses (FIG. 50), such as:

(i) CD38, an ectonucleotidase with various functions including regulation of adenosine signaling, adhesion, and transduction of activation and proliferation signals^{[162, 163}]. Given that purinergic receptors can be therapeutically targeted, it will be pertinent to test how CD39 and CD38 modulate ATP and purinergic signaling to influence the development and function of anti-tumor TRM cells (CD103⁺CD8⁺ TILs). Applicants will test functions of these targets.

(ii) KIR2DL4, upregulated in T_{RM}-high tumors, encodes the killer cell immunoglobulin-like receptor KIR2DL4, which has activating and inhibitory functions^{[164]} HLA-G, a non-classical MHC class I molecule, has been shown to engage KIR2DL4 and increase cytokine production by NK cells^{[165]}. Though the expression of HLA-G is highly restricted, several reports have shown its increased expression in tumor tissue, especially in lung cancer^{[166]}, so Applicants speculate that HLA-G in tumors may activate CTLs via the KIR2DL4 receptor to enhance their anti-tumor activities.

(iii) SIRPG encodes a member of the immunoglobulin superfamily of signal-regulatory proteins (SIRPs) that interact with the ubiquitously expressed CD47 molecule^{[167]}. Interestingly, SIRPG is the only member of the SIRP family that is expressed on T cells, and its interaction with CD47 expressed on APCs was shown to enhance T cell proliferation and IFN-γ production^{[168]}. Based on the increased expression of SIRPG transcripts in CD103^{high}CD8⁺ TILs, Applicants speculate that SIRPG may also serve as an important co-stimulatory molecule and its function could be exploited to enhance the anti-tumor function of CTLs.

More recently, Applicants performed additional studies in purified populations of TRM cells in lung and tumor tissue (FIG. 2-FIG. 5). These analyses defined a core set of genes commonly expressed in both lung and tumor TRM cells, including a number of novel genes whose expression was highly correlated with known tissue residency (T_{RM}) genes. Any of these genes may also be critically important for the development, trafficking or function of lung or lung tumor-infiltrating TRM cells. Some notable examples known or likely to have such functions are AMICA1⁶⁸, encoding JAML (junctional adhesion molecule-like), which contributes to the proliferation and cytokine release of skin-resident γδT cells; and SRGAP3, whose product functions in neuronal migration⁶⁷. Additional TRM candidate molecules that are associated with ‘immune hot’ tumors, M1^{hot} myeloid program, interferon-response signature in tumor cells, and finally responsiveness to anti-PD1 therapy will be tested.

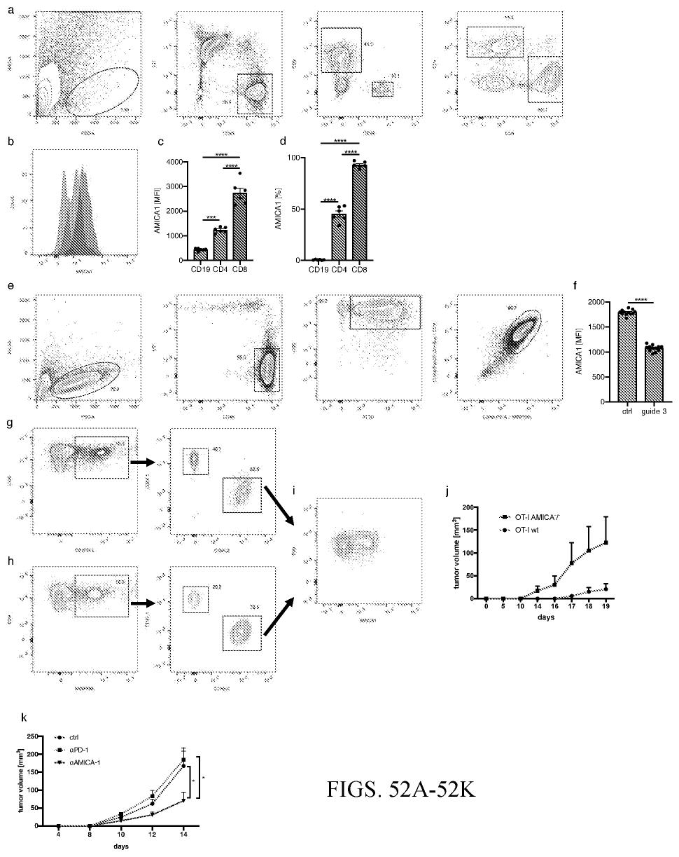

Additionally, Applicants have validated high protein expression of AMICA1 and found heightened expression in tumor infiltrating CD8 T cells, not only substantiating the RNA-seq data, but also highlighting CD8+ TILs as cellular targets of potential immunotherapy intervention. (FIG. 52) Applicants have also validated a knockout system specifically depleting AMICA1 in tumor antigen-specific CD8 T cells. By adoptively transferring these cells into tumor-bearing recipient mice, the Applicants discovered that although AMICA-1−/−CD8 cells efficiently migrate into the tumor micro environment, they fail to facilitate efficient anti-tumor effects compared with control CD8 T cells. These data indicate that a lack of AMICA1 expression specifically in CD8+ TILs ensues loss of functionality. Additionally, B16F10-OVA tumor-bearing mice were treated with either anti-PD-1, anti-AMICA-1 or isotype control antibodies. These data, shown in FIG. 52K further corroborate the previous results by illustrating that treatment with an agonistic anti-AMICA1 antibody significantly impedes tumor growth. The combination of this finding and previous data discussed herein suggests that this effect is mediated via stimulation of tumor infiltrating CD8+ T cells.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

TABLE 1
Gene

AMICA1
CD28H
CHN1
SPRY1
CD226
PTPN22
DUSP4
CLEC2D
KRT86
CD101
CD200R1

TABLE 2
Gene

AMICA (JAML)
SPRY1
CHN1
PAG1
PTPN22
DUSP4
ICOS
TNFRSF18 (GITR)
CD28H (TMIGD2)
CD226
TIGIT
KLRC1 (NKG2A)
KLRC2 (NKG2C)
CAPG
MYO1E
CLEC2B (AICL - Activation-Induced C-Type
Lectin)
CLECL1
TNFRSF9 (4-1BB/CD137)
TNFSF4 (OX-40L)
NR3C1
CD7
KLRD1 (CD94)
CLEC2D
ITM2A
VCAM1 (CD106)
KRT81
KRT86
CXCL13
CBLB
KLRC3 (NKG2-E)
KLRB1 (CD161)
CD101
CD109
CD200R1
SLA (SLAP)

TABLE 3
List of genes differentially expressed between Lung TRM compared to Lung non-TRM

| | log2FoldChange | pvalue | padj | Mean TPM Lung CD103neg | MeanTPM Lung CD103pos |
|---|---|---|---|---|---|
| ITGAE | 3.688320634 | 1.70E−59 | 1.97E−55 | 21.46150476 | 265.10205 |
| S1PR5 | −6.687936244 | 1.10E−42 | 6.36E−39 | 134.1027238 | 3.67714065 |
| CX3CR1 | −6.991370416 | 1.82E−36 | 7.04E−33 | 175.2148667 | 1.804877645 |
| S1PR1 | −3.641962229 | 1.06E−26 | 3.07E−23 | 239.1302906 | 19.1754866 |
| PRSS23 | −4.083087713 | 3.19E−26 | 7.40E−23 | 22.8481059 | 1.4572142 |
| FCRL3 | −5.450452146 | 3.69E−24 | 7.13E−21 | 56.6788171 | 8.42927178 |
| FGFBP2 | −5.23625676 | 2.83E−20 | 4.68E−17 | 287.5229123 | 7.5970008 |
| LINC00987 | −4.335283635 | 6.93E−20 | 1.00E−16 | 11.51608357 | 0.60433175 |
| ADGRG1 | −3.690227626 | 3.02E−19 | 3.90E−16 | 146.5809238 | 15.91066901 |
| ZNF683 | 2.840701441 | 2.39E−18 | 2.77E−15 | 91.602989 | 777.8943737 |
| LDLRAD4 | 2.720609868 | 4.83E−17 | 5.09E−14 | 4.93274 | 24.7280175 |
| PLEK | −3.953518263 | 5.97E−17 | 5.77E−14 | 325.5233143 | 31.67810321 |
| LIMD2 | −1.161393759 | 1.27E−16 | 1.13E−13 | 182.1111619 | 85.68926 |
| MIR4461 | 1.308404092 | 4.29E−16 | 3.55E−13 | 2173.675619 | 6586.9165 |
| KLRAP1 | −4.875420416 | 4.06E−15 | 3.14E−12 | 32.02053743 | 3.0425634 |
| PDGFD | −4.717712955 | 6.21E−15 | 4.50E−12 | 29.30154943 | 1.53936536 |
| C1orf21 | −4.088843226 | 1.51E−14 | 1.03E−11 | 13.63393756 | 0.99581828 |
| FCGR3A | −2.490323421 | 1.64E−14 | 1.06E−11 | 357.0966381 | 74.9510268 |
| SPRY1 | 4.365932648 | 3.96E−14 | 2.42E−11 | 3.749139257 | 82.8237395 |
| ABCC9 | 1.306250208 | 6.60E−14 | 3.83E−11 | 2.177677476 | 5.7461988 |
| OPHN1 | 1.698830765 | 8.04E−14 | 4.44E−11 | 7.505019524 | 22.160505 |
| LGR6 | −4.134216295 | 3.75E−13 | 1.98E−10 | 27.19662095 | 2.625316415 |
| SNTB2 | 2.065569735 | 4.19E−13 | 2.11E−10 | 3.784128857 | 13.9412381 |
| PLAC8 | −2.522266407 | 4.44E−13 | 2.15E−10 | 114.6815 | 26.4785965 |
| LOC102724699 | 1.780016048 | 6.86E−13 | 3.18E−10 | 8.024455238 | 27.9874615 |
| FEZ1 | −2.934653107 | 1.47E−12 | 6.55E−10 | 22.35391219 | 3.54457915 |
| KLRG1 | −2.873819675 | 1.88E−12 | 8.09E−10 | 469.6674938 | 74.7741715 |
| GPR155 | 2.047989691 | 3.24E−12 | 1.21E−09 | 7.462555429 | 24.60980145 |
| RNF130 | −2.989955573 | 3.08E−12 | 1.21E−09 | 110.1369376 | 19.8595257 |
| SELL | −3.329331038 | 3.22E−12 | 1.21E−09 | 507.2940325 | 55.0757787 |
| KLF3 | −3.67725202 | 3.02E−12 | 1.21E−09 | 62.23310952 | 8.94888858 |
| KLRC1 | 3.279902899 | 3.76E−12 | 1.36E−09 | 36.97543338 | 428.0637866 |
| MTRNR2L2 | 1.563127479 | 4.80E−12 | 1.64E−09 | 1391.110667 | 5314.522 |
| ZNF365 | −3.395971331 | 4.80E−12 | 1.64E−09 | 6.917136971 | 1.11013625 |
| PREX1 | 2.029428202 | 6.66E−12 | 2.21E−09 | 17.72572143 | 72.19956 |
| MTRNR2L8 | 1.466995835 | 6.86E−12 | 2.21E−09 | 544.3467143 | 1855.34295 |
| ARHGEF26-AS1 | 1.490358206 | 8.70E−12 | 2.66E−09 | 4.932213333 | 13.310475 |
| JUNB | −1.252749633 | 8.64E−12 | 2.66E−09 | 1745.32781 | 638.31095 |
| ITGA1 | 4.051013491 | 9.38E−12 | 2.78E−09 | 7.034438786 | 137.3742612 |
| PHLDA1 | 1.983873635 | 9.82E−12 | 2.78E−09 | 25.87213667 | 89.10818075 |
| RAP1GAP2 | −4.254360181 | 9.60E−12 | 2.78E−09 | 12.79643925 | 0.769949395 |
| A2M | −2.434435477 | 1.61E−11 | 4.44E−09 | 27.18622429 | 5.6312523 |
| LGALS3 | 1.474559911 | 1.91E−11 | 5.17E−09 | 541.8120429 | 685.16675 |
| PLP2 | 1.585554018 | 3.40E−11 | 8.96E−09 | 133.4817429 | 328.37947 |
| CLDN11 | 1.49888062 | 3.90E−11 | 1.01E−08 | 7.071935714 | 20.91596 |
| KIAA1551 | 1.53336279 | 5.04E−11 | 1.27E−08 | 136.1961962 | 372.187995 |
| PLEKHG3 | −3.801670968 | 5.17E−11 | 1.28E−08 | 16.75150993 | 1.15220756 |
| ITGB2 | −1.178163171 | 6.65E−11 | 1.61E−08 | 680.3715238 | 321.599435 |
| ADAM19 | 2.136052341 | 7.96E−11 | 1.89E−08 | 21.82406658 | 82.6178222 |
| MIR6723 | 1.364171561 | 1.27E−10 | 2.89E−08 | 5411.630952 | 15427.5075 |
| FGR | −3.427375695 | 1.25E−10 | 2.89E−08 | 203.5495476 | 27.5633367 |
| FAM169A | −3.933059942 | 3.05E−10 | 6.67E−08 | 9.554643095 | 1.556730675 |
| FMNL3 | 1.886848299 | 3.25E−10 | 6.99E−08 | 4.030502919 | 14.83709891 |
| MYO7A | 4.503614597 | 4.04E−10 | 8.53E−08 | 0.038636476 | 13.82020979 |
| LOC643406 | 1.080015874 | 4.92E−10 | 1.02E−07 | 8.94110381 | 21.4070425 |
| LAIR2 | −3.648996225 | 7.50E−10 | 1.53E−07 | 80.42070281 | 22.79628975 |
| LINC00536 | −2.30294596 | 1.18E−09 | 2.36E−07 | 4.489862762 | 1.01907325 |
| AMICA1 | 1.778550701 | 1.75E−09 | 3.45E−07 | 119.9607238 | 344.4711 |
| VIM | 1.494086613 | 1.96E−09 | 3.80E−07 | 491.6159762 | 1106.35245 |
| PRKDC | 1.514072515 | 2.07E−09 | 3.93E−07 | 25.9920519 | 71.954705 |
| PGK1 | 1.086986991 | 2.84E−09 | 5.22E−07 | 568.5674429 | 1142.7099 |
| GNLY | −2.081880512 | 2.82E−09 | 5.22E−07 | 3093.530095 | 635.69869 |
| VPS13C | 1.567869275 | 3.37E−09 | 6.11E−07 | 8.968918914 | 25.817943 |
| CLNK | 4.288010598 | 5.51E−09 | 9.83E−07 | 0.025489571 | 37.81930265 |
| IL12RB2 | 2.207617883 | 6.35E−09 | 1.12E−06 | 5.973771714 | 19.9212583 |
| MTRNR2L10 | 1.288450447 | 6.52E−09 | 1.13E−06 | 92.07810952 | 274.15068 |
| C5orf28 | −2.365273529 | 6.65E−09 | 1.13E−06 | 28.15868324 | 10.8994104 |
| KLRF1 | −3.513812967 | 7.27E−09 | 1.22E−06 | 193.5823511 | 14.4933614 |
| MTRNR2L6 | 1.362905237 | 8.10E−09 | 1.34E−06 | 9.190159048 | 29.3221775 |
| TMEM200A | 3.469977659 | 8.52E−09 | 1.39E−06 | 3.816969743 | 49.1354955 |
| MTRNR2L1 | 1.212300682 | 9.07E−09 | 1.46E−06 | 226.1222238 | 634.7212 |
| SH3BP5 | −2.89346707 | 9.37E−09 | 1.49E−06 | 13.15606714 | 2.10172946 |
| GSG2 | 3.128523686 | 1.02E−08 | 1.61E−06 | 0.161439895 | 8.00016295 |
| ANKRD28 | 2.439836494 | 1.20E−08 | 1.83E−06 | 14.10839 | 73.65266634 |
| DUSP2 | −1.186515607 | 1.20E−08 | 1.83E−06 | 1253.61149 | 578.04335 |
| MTRNR2L9 | 1.337497628 | 1.39E−08 | 2.10E−06 | 319.5508095 | 1052.6489 |
| CADM1 | −3.798923884 | 1.73E−08 | 2.58E−06 | 14.14056571 | 0.541758555 |
| USP28 | −2.800231732 | 1.87E−08 | 2.74E−06 | 46.590065 | 8.1924564 |
| CXCR6 | 2.074928075 | 1.95E−08 | 2.83E−06 | 215.3580623 | 799.8171578 |
| OXNAD1 | 1.165357916 | 2.04E−08 | 2.92E−06 | 51.84040952 | 126.460545 |
| LOC100129434 | 1.186595285 | 2.20E−08 | 3.08E−06 | 20.46000667 | 45.882185 |
| ICAM2 | −2.222167739 | 2.20E−08 | 3.08E−06 | 76.36366667 | 18.5619424 |
| PGLYRP2 | 3.318268163 | 2.67E−08 | 3.69E−06 | 1.056929062 | 7.5602649 |
| DNAJB1 | −1.413412514 | 2.70E−08 | 3.69E−06 | 802.5950476 | 348.781017 |
| EZH2 | 2.541688868 | 2.83E−08 | 3.82E−06 | 7.69050929 | 28.55026275 |
| CXCR2 | −2.872882644 | 3.15E−08 | 4.19E−06 | 25.0598669 | 3.1510207 |
| CMC1 | −2.098618623 | 3.54E−08 | 4.67E−06 | 334.9550048 | 76.791481 |
| KIR3DL2 | −3.16784706 | 4.66E−08 | 6.07E−06 | 44.4269131 | 5.5029878 |
| PELO | 2.512227676 | 4.82E−08 | 6.19E−06 | 14.90672521 | 80.21213 |
| RIPK2 | −2.592150871 | 4.85E−08 | 6.19E−06 | 51.12927 | 12.00812765 |
| LITAF | −1.186041133 | 5.17E−08 | 6.53E−06 | 489.3827143 | 224.55413 |
| SRSF2 | 1.073461158 | 5.71E−08 | 7.05E−06 | 280.4715619 | 643.1785 |
| ITGAM | −3.288179636 | 5.69E−08 | 7.05E−06 | 20.78114599 | 9.55108064 |
| SPON2 | −3.08944003 | 5.92E−08 | 7.23E−06 | 68.93419057 | 8.0418225 |
| EOMES | −2.350328769 | 5.99E−08 | 7.24E−06 | 96.09277629 | 25.55344615 |
| ELOVL5 | 1.194140558 | 6.30E−08 | 7.53E−06 | 77.90133333 | 188.919585 |
| ABHD11 | −2.246196046 | 8.68E−08 | 1.03E−05 | 28.72630952 | 8.1834175 |
| RAB12 | −1.056317965 | 8.94E−08 | 1.05E−05 | 315.4402381 | 159.223475 |
| SH2D1B | −3.653555023 | 9.20E−08 | 1.07E−05 | 22.65793566 | 1.67038849 |
| EGR1 | 1.328069038 | 1.11E−07 | 1.25E−05 | 90.65356333 | 180.64925 |
| RCBTB2 | −2.761362657 | 1.26E−07 | 1.40E−05 | 65.87080476 | 19.41631984 |
| ATP8B4 | 3.615872085 | 1.31E−07 | 1.43E−05 | 0.344809071 | 10.5492769 |
| RGS1 | 1.374030196 | 1.31E−07 | 1.43E−05 | 756.459619 | 1916.0517 |
| ASCL2 | −3.713952755 | 1.33E−07 | 1.44E−05 | 10.87647238 | 0.357964 |
| GPR25 | 3.450094942 | 1.71E−07 | 1.84E−05 | 3.260915429 | 49.20251905 |
| SMURF2 | 1.900071442 | 2.11E−07 | 2.22E−05 | 13.87433392 | 39.638273 |
| LOC101927374 | 1.11281412 | 2.13E−07 | 2.22E−05 | 12.26230476 | 27.5276295 |
| KIAA0825 | 1.793395112 | 2.26E−07 | 2.34E−05 | 9.062423619 | 28.9025909 |
| LARP1 | 1.334004962 | 2.41E−07 | 2.45E−05 | 9.35280619 | 28.1623345 |
| SNAP23 | −1.228426954 | 2.47E−07 | 2.49E−05 | 137.3034905 | 72.05703075 |
| CISH | 1.627753438 | 2.82E−07 | 2.79E−05 | 80.86795833 | 285.2522457 |
| ZNF559 | −3.025142135 | 2.84E−07 | 2.79E−05 | 25.17488419 | 6.09491092 |
| SLC35D2 | 2.110097143 | 2.94E−07 | 2.84E−05 | 5.033894524 | 21.18080975 |
| FAM65B | −1.600964159 | 2.94E−07 | 2.84E−05 | 97.72626048 | 42.184457 |
| LOC100130093 | 2.384781043 | 3.43E−07 | 3.23E−05 | 4.471636 | 23.500032 |
| FAM129A | 1.202394244 | 3.47E−07 | 3.24E−05 | 33.137718 | 72.1486845 |
| ITM2C | 1.911897049 | 3.58E−07 | 3.32E−05 | 67.57806086 | 250.0731207 |
| ARHGAP25 | −1.817060321 | 3.65E−07 | 3.36E−05 | 152.4628095 | 69.0699945 |
| LOC100130872 | −1.991189374 | 3.75E−07 | 3.43E−05 | 16.75408481 | 6.593254745 |
| ZFAS1 | 1.199358326 | 3.85E−07 | 3.49E−05 | 83.68128333 | 177.09171 |
| CYBB | −1.793527414 | 3.93E−07 | 3.53E−05 | 64.10834148 | 11.9938415 |
| PZP | −2.888358053 | 4.16E−07 | 3.71E−05 | 8.12034131 | 3.009308975 |
| HIST1H2BK | 2.05603388 | 4.22E−07 | 3.73E−05 | 39.28850138 | 123.0758285 |
| MALT1 | 2.349361739 | 4.71E−07 | 4.11E−05 | 12.3671099 | 42.27466225 |
| RAPGEF6 | 1.241140351 | 4.89E−07 | 4.20E−05 | 28.62101667 | 67.59247 |
| PRR5L | −2.720570835 | 4.88E−07 | 4.20E−05 | 28.4360878 | 8.383760905 |
| TM9SF3 | 1.719606775 | 6.14E−07 | 5.24E−05 | 22.233558 | 74.4089565 |
| LINC00612 | −2.822238193 | 6.87E−07 | 5.82E−05 | 13.02071243 | 2.1673256 |
| RASGRP2 | −1.960339855 | 8.11E−07 | 6.82E−05 | 104.0838905 | 30.4704815 |
| MCF2L2 | 1.77037345 | 8.17E−07 | 6.82E−05 | 1.075314152 | 3.41337215 |
| ADD3 | −1.398797561 | 8.31E−07 | 6.89E−05 | 159.9318571 | 75.43510185 |
| XCL1 | 2.033544975 | 9.53E−07 | 7.79E−05 | 30.51550386 | 141.3891085 |
| MIR155HG | 2.718579249 | 1.20E−06 | 9.74E−05 | 8.770128333 | 68.19258875 |
| TTC39B | −1.95836329 | 1.21E−06 | 9.74E−05 | 28.75757238 | 6.09487136 |
| ZNF44 | −2.534590645 | 1.29E−06 | 0.00010334 | 39.33668429 | 12.8156669 |
| HNRNPD | 1.22436894 | 1.34E−06 | 0.00010555 | 42.86327286 | 106.433995 |
| TTC38 | −1.605359191 | 1.34E−06 | 0.00010555 | 80.5307819 | 28.116408 |
| FCMR | −1.777542843 | 1.36E−06 | 0.00010696 | 200.2598944 | 80.13699245 |
| LINC00504 | 1.081582043 | 1.55E−06 | 0.00012107 | 26.64122286 | 62.159235 |
| SHOX | 1.380820768 | 1.67E−06 | 0.00012931 | 0.463633286 | 1.33043075 |
| PDE4D | 1.630857417 | 1.69E−06 | 0.00012979 | 10.41079262 | 30.78342656 |
| MORC2-AS1 | −1.720968756 | 1.89E−06 | 0.00014451 | 63.34037095 | 20.5457955 |
| CCR7 | −2.464404119 | 1.95E−06 | 0.00014814 | 239.9860878 | 49.3558936 |
| DOK2 | −1.470162395 | 2.02E−06 | 0.0001519 | 324.6862381 | 127.6544189 |
| ORMDL2 | 1.069442343 | 2.06E−06 | 0.00015351 | 113.456781 | 226.26465 |
| RIN2 | −2.966123994 | 2.09E−06 | 0.00015443 | 9.440358186 | 0.199756825 |
| LDHA | 1.049322824 | 2.11E−06 | 0.00015527 | 570.599381 | 1162.148 |
| SFMBT2 | 1.948897349 | 2.13E−06 | 0.00015533 | 2.63146119 | 9.8511154 |
| UGDH-AS1 | 1.248838088 | 2.28E−06 | 0.00016539 | 19.08877381 | 48.18311 |
| IVNS1ABP | 1.275070942 | 2.36E−06 | 0.00017027 | 148.0731952 | 400.824054 |
| RASA3 | −1.915627975 | 2.40E−06 | 0.00017163 | 66.32533333 | 21.9665584 |
| MPI | −2.353326054 | 2.43E−06 | 0.00017318 | 28.3699084 | 6.88767175 |
| SIRT2 | −1.499552785 | 2.62E−06 | 0.00018564 | 115.2741905 | 55.65131365 |
| LILRB1 | −2.073507487 | 2.81E−06 | 0.00019727 | 22.42856014 | 6.9053577 |
| CDK6 | 1.800012015 | 2.84E−06 | 0.00019876 | 17.0048809 | 62.511125 |
| CEP350 | 1.716329718 | 2.89E−06 | 0.00020049 | 10.43966743 | 38.48460117 |
| CXCR3 | 1.429112179 | 3.21E−06 | 0.00022162 | 82.86109976 | 221.8926819 |
| GPM6A | 1.873719438 | 3.39E−06 | 0.00023287 | 0.336675162 | 2.202836 |
| SWSAP1 | −1.54571358 | 3.51E−06 | 0.00023827 | 14.06452133 | 4.7382231 |
| NUPR1 | −2.102624894 | 3.61E−06 | 0.00024356 | 110.0524586 | 23.0499858 |
| SKIL | 1.356725934 | 3.82E−06 | 0.00025447 | 36.2539719 | 73.784065 |
| NUAK2 | −1.977103288 | 3.84E−06 | 0.00025447 | 13.48667505 | 3.09541505 |
| TANC2 | 3.089882387 | 4.04E−06 | 0.00026327 | 0.553212024 | 5.12779167 |
| KLF7 | −3.106136118 | 4.04E−06 | 0.00026327 | 6.978648795 | 0.610988815 |
| CPNE7 | 2.987748945 | 4.14E−06 | 0.00026804 | 3.079236524 | 27.6457933 |
| PCNX | 1.679911819 | 4.23E−06 | 0.00027251 | 10.39656719 | 32.2845755 |
| ACTN1 | −2.74953546 | 4.27E−06 | 0.00027343 | 42.51342762 | 8.75996527 |
| CUTC | −1.221976687 | 4.67E−06 | 0.00029475 | 129.3552048 | 69.6624427 |
| GIMAP4 | −1.414835705 | 4.65E−06 | 0.00029475 | 445.6157337 | 190.8786594 |
| PHF14 | 1.553582494 | 4.75E−06 | 0.00029802 | 5.411563848 | 12.4822209 |
| LPAR6 | −1.548072876 | 4.78E−06 | 0.00029823 | 71.46590388 | 27.4985452 |
| COTL1 | 1.219750696 | 5.12E−06 | 0.00031774 | 444.2532048 | 838.82325 |
| LDLRAP1 | −1.734939687 | 5.18E−06 | 0.00031968 | 81.61549524 | 28.7617736 |
| RNF44 | 1.68910313 | 5.26E−06 | 0.00032294 | 16.61935648 | 59.77938 |
| OTUD5 | 1.846177309 | 5.54E−06 | 0.00033832 | 19.55586529 | 71.178468 |
| FAM49A | −2.526705269 | 5.63E−06 | 0.00034185 | 29.38951762 | 6.759340495 |
| KIF5C | 2.442095274 | 5.76E−06 | 0.00034817 | 1.484135657 | 9.82658695 |
| PCBP1 | 1.474990375 | 6.32E−06 | 0.00037993 | 449.108119 | 964.3716 |
| B4GALT5 | 1.766414298 | 6.54E−06 | 0.00039088 | 3.2958552 | 7.8889765 |
| PERP | 1.746555154 | 6.89E−06 | 0.00040982 | 20.26485792 | 93.237233 |
| ILF3-AS1 | −1.401809132 | 6.97E−06 | 0.00041245 | 48.10910714 | 23.90241965 |
| CCDC144B | 1.520739947 | 7.58E−06 | 0.0004459 | 4.185302905 | 12.8182025 |
| RAC1 | 1.105059877 | 7.61E−06 | 0.0004459 | 139.2755238 | 233.57116 |
| RPS6KA3 | 1.595115819 | 8.30E−06 | 0.0004838 | 12.37155557 | 34.417014 |
| GIMAP7 | −1.4037857 | 8.41E−06 | 0.00048791 | 764.0689048 | 367.0225432 |
| DUSP4 | 2.262161797 | 8.48E−06 | 0.00048941 | 12.86056076 | 46.99192715 |
| RPL5 | 1.176379227 | 9.00E−06 | 0.00051411 | 1708.037571 | 3358.533 |
| DEGS1 | 1.103804675 | 9.31E−06 | 0.00052667 | 146.0186857 | 277.532665 |
| LRP1 | 2.494030823 | 9.43E−06 | 0.00053103 | 1.215511952 | 4.593950775 |
| FRMD4B | 1.967481113 | 9.81E−06 | 0.00054713 | 17.27852171 | 39.86186793 |
| DDX60 | 1.740019415 | 1.02E−05 | 0.00056735 | 14.20610748 | 59.79492642 |
| ARHGEF1 | −1.291779535 | 1.05E−05 | 0.00058041 | 212.2836 | 109.3370274 |
| TMPRSS3 | 2.104017045 | 1.09E−05 | 0.0005971 | 0.990603448 | 7.09934565 |
| ARHGAP35 | 2.020567437 | 1.13E−05 | 0.00061628 | 7.980023814 | 32.97274923 |
| SPATS2L | 1.568699983 | 1.15E−05 | 0.00061923 | 7.175204857 | 18.4375802 |
| ZNF100 | 1.727488104 | 1.16E−05 | 0.00062335 | 1.838728252 | 14.04667465 |
| CTSO | −2.394862763 | 1.18E−05 | 0.00063276 | 87.34779524 | 28.05486479 |
| SRGAP3 | 3.083987351 | 1.21E−05 | 0.00064522 | 0.327200095 | 6.54159999 |
| CHN1 | 3.016328106 | 1.29E−05 | 0.00068228 | 3.889638771 | 26.56366172 |
| ASB6 | −1.409994218 | 1.29E−05 | 0.00068228 | 24.44135762 | 10.9425055 |
| NT5E | −2.612624338 | 1.49E−05 | 0.00077849 | 14.35977676 | 3.21892007 |
| PUS3 | −3.022511352 | 1.49E−05 | 0.00077849 | 14.78100877 | 0.55856775 |
| CHMP6 | −1.65237349 | 1.58E−05 | 0.0008245 | 90.8889381 | 32.4718011 |
| LOC102723824 | 2.220309075 | 1.61E−05 | 0.00083527 | 8.376731619 | 17.913781 |
| SRSF8 | −1.272937322 | 1.64E−05 | 0.00084665 | 71.34348429 | 57.41924175 |
| BIN2 | −1.259196899 | 1.67E−05 | 0.00085116 | 491.3041429 | 219.0422681 |
| ZFYVE28 | 2.300538964 | 1.70E−05 | 0.00086536 | 4.428132524 | 19.4064535 |
| EHBP1L1 | 1.996718023 | 1.73E−05 | 0.00087228 | 7.180443862 | 25.8848635 |
| PTRH1 | −2.291611353 | 1.72E−05 | 0.00087228 | 71.97044286 | 18.2559359 |
| ASCC3 | 1.525897011 | 1.80E−05 | 0.00090279 | 8.081438562 | 24.3278745 |
| VDAC1 | 1.019841564 | 1.81E−05 | 0.00090279 | 141.0238476 | 235.935545 |
| ZNF18 | −2.631248872 | 1.81E−05 | 0.00090279 | 28.62923619 | 7.869980955 |
| KIF21B | 2.166012602 | 1.83E−05 | 0.00090745 | 4.438138 | 20.382474 |
| LAG3 | 1.22701912 | 1.88E−05 | 0.00092214 | 87.38349743 | 217.4806655 |
| ARHGAP11A | 1.444394843 | 1.89E−05 | 0.00092638 | 2.611797762 | 6.46693175 |
| PARP3 | −2.89694382 | 2.07E−05 | 0.00100857 | 19.20667543 | 2.15505298 |
| C16orf89 | −2.402216829 | 2.23E−05 | 0.00107835 | 16.13956348 | 0.8268643 |
| MTPAP | −1.257160683 | 2.24E−05 | 0.00107991 | 28.74861643 | 13.57233135 |
| TSPAN32 | −1.711545059 | 2.28E−05 | 0.0010934 | 99.66786667 | 46.714372 |
| RASSF4 | −2.712424641 | 2.36E−05 | 0.00112716 | 38.27995619 | 7.67994325 |
| HUWE1 | 1.478732178 | 2.38E−05 | 0.00113113 | 8.3189656 | 27.50419295 |
| DOCK5 | 2.625035961 | 2.45E−05 | 0.00115108 | 1.4117016 | 6.88680025 |
| TNF | 1.537622653 | 2.43E−05 | 0.00115108 | 8.510453143 | 19.906433 |
| ATP2B1 | 1.232541924 | 2.45E−05 | 0.00115108 | 53.28257048 | 120.203785 |
| LZTS1 | 1.89060308 | 2.48E−05 | 0.00116116 | 1.530348495 | 6.11112885 |
| CCL28 | 1.515771195 | 2.53E−05 | 0.00117224 | 6.587727714 | 13.8866925 |
| POLH | 1.362294764 | 2.52E−05 | 0.00117224 | 4.764934857 | 10.2461875 |
| PLXDC1 | 1.910398886 | 2.56E−05 | 0.00117916 | 7.218845452 | 22.21014939 |
| SLC38A2 | 1.119051382 | 2.56E−05 | 0.00117916 | 53.13309048 | 117.44348 |
| ADCY3 | 2.560946699 | 2.64E−05 | 0.00120959 | 0.627494476 | 5.456133665 |
| MTX3 | 1.669116918 | 2.69E−05 | 0.0012236 | 3.042661571 | 14.24616695 |
| ZNRF1 | 2.828733845 | 3.09E−05 | 0.00139642 | 0.810231405 | 6.82966755 |
| AKR1C3 | −2.587366759 | 3.30E−05 | 0.00147422 | 35.08770348 | 11.6727318 |
| KIR3DL1 | −2.774117252 | 3.30E−05 | 0.00147422 | 39.17617897 | 4.93104205 |
| KMT2D | 2.0488088 | 3.44E−05 | 0.00152285 | 1.4962252 | 7.7576795 |
| IFNG | 1.783908689 | 3.48E−05 | 0.00153289 | 82.55949667 | 362.1978226 |
| UBE2F | −1.718368644 | 3.49E−05 | 0.00153289 | 55.71082381 | 21.82958125 |
| BLOC1S3 | −1.737490695 | 3.69E−05 | 0.0016159 | 37.0366489 | 12.4104362 |
| ZNF350 | −2.20269071 | 3.77E−05 | 0.00164559 | 39.55514852 | 17.20023995 |
| KIR2DL3 | −2.735915385 | 3.88E−05 | 0.0016848 | 29.8276564 | 2.69000165 |
| CD14 | −2.112206031 | 3.91E−05 | 0.00169426 | 74.05469481 | 16.6984343 |
| SLC1A5 | 1.534783918 | 3.98E−05 | 0.0017162 | 33.13000886 | 96.9581249 |
| RBMS1 | 1.704885397 | 4.26E−05 | 0.00180177 | 42.58026438 | 148.25084 |
| NKTR | 1.080979938 | 4.25E−05 | 0.00180177 | 34.011989 | 82.13539 |
| PEX16 | −1.01884714 | 4.27E−05 | 0.00180177 | 61.71854743 | 36.40784535 |
| RAP2B | −1.273692809 | 4.25E−05 | 0.00180177 | 80.36729524 | 34.5275585 |
| TCOF1 | −1.940096541 | 4.24E−05 | 0.00180177 | 20.13614476 | 8.973999545 |
| HIVEP3 | −2.001507989 | 4.39E−05 | 0.00184472 | 7.941141333 | 3.032539515 |
| KEAP1 | −1.22615691 | 4.45E−05 | 0.00186341 | 64.71328551 | 31.5632675 |
| ARRDC3 | −1.17054195 | 4.54E−05 | 0.00189564 | 211.8614238 | 112.5465397 |
| MXRA7 | 1.658912627 | 4.91E−05 | 0.00204173 | 3.266608619 | 15.1301335 |
| ESYT2 | 1.514083794 | 5.16E−05 | 0.00212136 | 12.05781967 | 47.540445 |
| NMUR1 | −1.773761089 | 5.15E−05 | 0.00212136 | 16.83415457 | 5.56423905 |
| LINC00892 | 2.424521999 | 5.18E−05 | 0.0021239 | 24.90726819 | 170.14904 |
| EIF2B3 | −1.937296892 | 5.22E−05 | 0.00213377 | 62.28575714 | 26.8829665 |
| UBA7 | −1.274777668 | 5.25E−05 | 0.00213647 | 89.77450238 | 52.6846935 |
| OGFRL1 | 2.379697737 | 5.32E−05 | 0.00215872 | 3.896613762 | 30.0878519 |
| NOSIP | −1.206988658 | 5.58E−05 | 0.00224595 | 172.3891771 | 90.073862 |
| APOL1 | −1.660848056 | 5.69E−05 | 0.00228344 | 14.78162748 | 5.5118258 |
| NKX3-1 | −2.804697893 | 5.79E−05 | 0.00231464 | 5.651472695 | 0.701179405 |
| ZNF441 | −2.454868826 | 5.97E−05 | 0.00236312 | 8.393922424 | 2.879041465 |
| YBX3 | 1.716666696 | 6.04E−05 | 0.00238334 | 24.78591295 | 61.299884 |
| PARD6G | 1.312295036 | 6.07E−05 | 0.00238688 | 4.132179238 | 12.0712125 |
| LYSMD1 | −2.924027783 | 6.10E−05 | 0.00239096 | 6.796088933 | 0.4503092 |
| KPNA4 | 1.45711023 | 6.13E−05 | 0.00239269 | 11.01992676 | 29.1450691 |
| TMEM212 | 1.058248599 | 6.45E−05 | 0.00250388 | 13.60060286 | 32.280461 |
| CTLA4 | 1.435048574 | 6.53E−05 | 0.00252477 | 19.54678733 | 68.7486734 |
| MYADM | 1.060424765 | 6.59E−05 | 0.00253937 | 53.35683857 | 123.7034305 |
| KLHL5 | 1.772588118 | 6.64E−05 | 0.00254182 | 2.376725143 | 9.9981583 |
| PIP4K2A | −1.288193495 | 6.63E−05 | 0.00254182 | 967.4229524 | 424.5596301 |
| KLHL6 | 1.598790907 | 6.70E−05 | 0.00255588 | 5.091936467 | 12.3455271 |
| C15orf53 | 2.610180897 | 6.95E−05 | 0.00262468 | 2.301744476 | 24.90123835 |
| DAPK2 | 1.912434282 | 6.92E−05 | 0.00262468 | 9.456853214 | 36.7584622 |
| TPST2 | −1.273052932 | 7.03E−05 | 0.00264731 | 263.7426048 | 125.121244 |
| TMEM41A | −1.720382667 | 7.10E−05 | 0.00265702 | 28.74279305 | 15.40182055 |
| PLAUR | −1.417464169 | 7.38E−05 | 0.002748 | 271.6738353 | 50.50656925 |
| RBPJ | 1.574107492 | 7.44E−05 | 0.00275597 | 65.66283667 | 211.5952325 |
| LRRC37A3 | 2.074130192 | 7.55E−05 | 0.00279009 | 0.480966952 | 2.98394201 |
| EPAS1 | 2.318444592 | 7.60E−05 | 0.00279574 | 6.038517781 | 26.05039855 |
| YIPF6 | −1.816114456 | 7.61E−05 | 0.00279574 | 12.21552449 | 5.799607305 |
| TMED1 | −1.817944234 | 7.80E−05 | 0.00285387 | 44.66887619 | 12.76184885 |
| NCOA7 | 1.293829014 | 7.85E−05 | 0.00286513 | 11.85079605 | 33.3728845 |
| TRNT1 | −1.56957745 | 8.10E−05 | 0.00294561 | 41.74942476 | 21.60956825 |
| PDCD1 | 1.960356621 | 8.23E−05 | 0.00298567 | 41.06805581 | 134.177045 |
| KRT86 | 2.801705688 | 8.31E−05 | 0.00300334 | 0.77714719 | 21.05319575 |
| CAPG | 1.160396095 | 8.39E−05 | 0.0030223 | 604.9114381 | 570.63815 |
| CYP51A1 | 1.217984916 | 8.92E−05 | 0.00318443 | 52.71879524 | 115.478695 |
| SQLE | 1.651331611 | 9.30E−05 | 0.00330959 | 23.50773952 | 69.026012 |
| EPB41 | −1.085276175 | 9.69E−05 | 0.00343927 | 35.59453905 | 24.4307317 |
| SNRNP35 | −1.380326304 | 9.96E−05 | 0.00352319 | 32.07545762 | 15.3377564 |
| PLEKHO1 | 2.101609708 | 0.00010076 | 0.00355319 | 10.75456533 | 34.77226 |
| TFDP2 | −1.074732513 | 0.00010209 | 0.00358918 | 13.8775519 | 7.722759 |
| SLC34A2 | −2.614315405 | 0.00010472 | 0.00365947 | 12.70014439 | 1.766653745 |
| MYC | −1.570814137 | 0.00010637 | 0.00370606 | 58.92450929 | 22.44566625 |
| TMED5 | 1.08124454 | 0.00010738 | 0.00372154 | 29.16007667 | 50.03997411 |
| NTPCR | −2.395567938 | 0.00010746 | 0.00372154 | 64.32159367 | 21.7462204 |
| WNK1 | 1.318502857 | 0.00010895 | 0.00376211 | 9.170893619 | 27.9849355 |
| XIST | 1.767227681 | 0.00010955 | 0.00377155 | 1.196562429 | 6.024847915 |
| SPC25 | 1.170654762 | 0.00011181 | 0.00383787 | 9.154391429 | 23.645568 |
| DENR | −1.22922827 | 0.00011379 | 0.00389424 | 73.8563619 | 47.0460279 |
| KIF11 | 1.821662005 | 0.00011551 | 0.00390267 | 2.443237967 | 9.257986 |
| GOLIM4 | 1.777868817 | 0.00011534 | 0.00390267 | 30.93483438 | 62.605315 |
| EPHA1-AS1 | 1.589736701 | 0.00011571 | 0.00390267 | 2.328378471 | 8.8394592 |
| SORL1 | −1.124650308 | 0.00011568 | 0.00390267 | 32.53818476 | 15.6531715 |
| INIP | −1.222448299 | 0.00011929 | 0.00401168 | 91.57427238 | 52.308057 |
| FAM228B | −2.041900144 | 0.00011983 | 0.00401813 | 13.19441343 | 5.38700615 |
| UAP1L1 | 1.889609098 | 0.00012291 | 0.00410963 | 2.936810938 | 12.80484475 |
| SMG9 | −2.185540007 | 0.00012737 | 0.00423412 | 25.12799343 | 8.71863 |
| TUBA1C | 1.655052435 | 0.0001324 | 0.0043889 | 25.54587348 | 56.705964 |
| LINC00341 | 1.789049608 | 0.00013297 | 0.00439536 | 12.578937 | 45.24209 |
| CBFB | 1.571033412 | 0.00013589 | 0.00445864 | 68.46266429 | 222.2403375 |
| POLR2E | −1.2088594 | 0.00013715 | 0.00448244 | 101.8120714 | 47.35464315 |
| ATL2 | 1.645098431 | 0.00013893 | 0.00452778 | 6.926690762 | 19.8609125 |
| CD27 | −1.282713519 | 0.00014204 | 0.00461599 | 149.9836976 | 69.1602188 |
| KCNC4 | 2.525809224 | 0.00014404 | 0.00462919 | 2.09133519 | 9.068993435 |
| KLF13 | 1.405276951 | 0.00014394 | 0.00462919 | 3.886962667 | 12.13907515 |
| GUSB | −1.087842847 | 0.00014291 | 0.00462919 | 127.3000857 | 59.46871275 |
| LPCAT1 | −1.887466956 | 0.00014327 | 0.00462919 | 50.49580452 | 12.65491646 |
| TBC1D25 | −1.991487663 | 0.00014464 | 0.00463568 | 22.69922333 | 7.652385165 |
| EFNA5 | −1.780188118 | 0.00014796 | 0.00472898 | 8.80737419 | 2.9897214 |
| LDB1 | 2.211714353 | 0.0001558 | 0.0049523 | 12.27335562 | 46.658046 |
| GSN | −1.856153002 | 0.00015695 | 0.00497514 | 155.9601187 | 24.92560408 |
| AGTPBP1 | −1.190748054 | 0.00015767 | 0.0049843 | 28.04230333 | 17.07175222 |
| CST7 | −1.279211634 | 0.0001628 | 0.00510307 | 1822.877929 | 814.3378435 |
| GZMK | −1.465391434 | 0.00016271 | 0.00510307 | 1090.510599 | 482.9047815 |
| RAB3D | −1.646487305 | 0.00016243 | 0.00510307 | 12.54113167 | 5.76880282 |
| FAM65A | −2.15466771 | 0.00016318 | 0.00510307 | 12.80896311 | 2.763608925 |
| DENND6A | −1.156167299 | 0.00016556 | 0.00516338 | 20.3185 | 12.27502635 |
| THOC3 | −1.44634318 | 0.00016754 | 0.00519729 | 9.395982857 | 5.46055835 |
| SLC7A5P2 | 1.461890782 | 0.00016864 | 0.00521747 | 14.76301557 | 41.072381 |
| GNB1 | 1.595767092 | 0.00017132 | 0.00528383 | 23.07147667 | 81.62131885 |
| CCDC65 | −2.34098191 | 0.00017364 | 0.00532954 | 29.07540152 | 6.60117545 |
| ZNF786 | −1.977944939 | 0.00017677 | 0.00541118 | 14.74838324 | 4.00298737 |
| TRIM24 | 2.423827168 | 0.00018028 | 0.00547555 | 3.644654986 | 26.3156509 |
| CARS2 | 1.693420689 | 0.00018023 | 0.00547555 | 20.01825719 | 53.228753 |
| CD226 | 2.11664127 | 0.00018211 | 0.0055145 | 20.47994643 | 94.98832335 |
| PLEKHJ1 | −1.42449896 | 0.00018252 | 0.0055145 | 111.3896667 | 45.58957835 |
| ADRB2 | −1.990923947 | 0.00018579 | 0.00559893 | 133.6191638 | 49.61041635 |
| MINA | −1.917614858 | 0.00018639 | 0.00560232 | 10.84250714 | 3.72207806 |
| IL11RA | −2.083504821 | 0.00018875 | 0.0056502 | 22.64916552 | 7.2149125 |
| DPEP2 | −2.205657439 | 0.00019126 | 0.00570442 | 43.10033762 | 17.71765105 |
| LOC440434 | 1.839111218 | 0.00019544 | 0.00581394 | 2.232405714 | 8.04353135 |
| C1RL-AS1 | 1.925490425 | 0.00020441 | 0.00604984 | 0.764437424 | 2.0629908 |
| SLAMF1 | 1.97751179 | 0.00020791 | 0.00613785 | 34.87498443 | 131.8489772 |
| PDE4A | 1.514702226 | 0.00021021 | 0.0061774 | 8.788954519 | 22.24409349 |
| TRAF3IP3 | −1.023757033 | 0.00021031 | 0.0061774 | 175.9603465 | 110.8125055 |
| SEL1L3 | 1.447604044 | 0.00022308 | 0.00651921 | 34.67522048 | 105.587455 |
| DOCK10 | 1.025366504 | 0.00022731 | 0.00662638 | 22.75661862 | 55.525593 |
| AGO2 | 1.913492388 | 0.0002291 | 0.00665622 | 6.69428181 | 29.835073 |
| APOOL | −1.4599624 | 0.00022994 | 0.00665622 | 9.866991905 | 4.9035758 |
| SLC4A1AP | −1.500967401 | 0.00023006 | 0.00665622 | 60.90765484 | 30.57019365 |
| GLYR1 | −1.493452199 | 0.00023127 | 0.00665804 | 50.69578571 | 23.97702565 |
| TESK1 | 2.335172957 | 0.00023342 | 0.00667018 | 3.665402619 | 25.04709 |
| SLC7A5 | 1.396734709 | 0.00023283 | 0.00667018 | 26.18238476 | 61.6948623 |
| HMHA1 | −1.095094418 | 0.00023328 | 0.00667018 | 153.4711905 | 82.52380625 |
| NR2C2 | 1.98376907 | 0.00023918 | 0.00681814 | 2.334334629 | 8.99618895 |
| AP4B1-AS1 | 1.303155865 | 0.00024286 | 0.00690603 | 3.367507905 | 10.55503925 |
| UNKL | −1.80143226 | 0.00024644 | 0.00697362 | 3.032874143 | 1.82100178 |
| ING3 | −1.406744774 | 0.00024721 | 0.00697847 | 56.85451143 | 28.07718918 |
| ANKRD32 | 1.443730347 | 0.00025762 | 0.00723698 | 33.46615714 | 104.99121 |
| NPIPB5 | 1.526741654 | 0.00026067 | 0.00728736 | 2.765647476 | 6.606986 |
| DOK6 | 2.513396625 | 0.00026462 | 0.00737965 | 0.146552833 | 2.537527625 |
| SNX27 | −1.40073971 | 0.00026524 | 0.00737965 | 37.33825619 | 15.8946053 |
| BBS2 | −1.950926604 | 0.00026753 | 0.0074255 | 74.32054286 | 26.4182932 |
| RASA1 | 1.354757826 | 0.00027325 | 0.00756612 | 13.71942938 | 31.58191005 |
| SLC39A11 | −1.598648182 | 0.00029385 | 0.00809794 | 35.99148971 | 14.80168625 |
| PITPNA | 2.312434612 | 0.0002998 | 0.0082228 | 3.488508 | 21.7398403 |
| RAD52 | −1.50567048 | 0.00030556 | 0.00836103 | 9.932621324 | 4.26503812 |
| HRSP12 | −2.383100619 | 0.00032349 | 0.00881022 | 27.23581476 | 9.8728184 |
| SMG1P3 | 1.200042025 | 0.00032556 | 0.00881529 | 1.363507952 | 2.71159545 |
| RRP7A | −1.165768141 | 0.00032596 | 0.00881529 | 39.24837762 | 20.3423858 |
| POLR3H | −1.67593178 | 0.00032576 | 0.00881529 | 13.8710933 | 6.146357295 |
| DHFRL1 | −2.359797868 | 0.00032712 | 0.00882622 | 5.099411576 | 0.47705205 |
| TYMP | 1.176092072 | 0.00033212 | 0.00894032 | 87.24913762 | 104.307335 |
| LPCAT3 | −1.27735195 | 0.00034009 | 0.0091335 | 79.39185714 | 48.14817045 |
| C5AR1 | −1.851391779 | 0.00034576 | 0.00926439 | 27.82386229 | 11.66871945 |
| ATF3 | 2.056842456 | 0.00034687 | 0.00927285 | 10.44020133 | 37.6365183 |
| CEP97 | 1.50396083 | 0.00035088 | 0.0093585 | 3.274631229 | 9.616456 |
| KIAA0513 | 1.368258293 | 0.00035513 | 0.00940686 | 3.681573657 | 5.3122714 |
| KIAA0020 | −1.159965895 | 0.00035504 | 0.00940686 | 73.4876049 | 41.18979315 |
| CCR6 | 1.511032115 | 0.00035676 | 0.0094153 | 14.34848795 | 23.36445165 |
| CPSF2 | 1.241995889 | 0.00035947 | 0.0094423 | 10.72023305 | 27.40330205 |
| METTL21A | 1.094244219 | 0.00035972 | 0.0094423 | 14.63200143 | 35.4652285 |
| NUPL2 | −1.832195681 | 0.00036147 | 0.00946668 | 51.23907567 | 20.4746829 |
| ASTN2 | 1.069590281 | 0.00036295 | 0.0094842 | 5.929357857 | 12.6587515 |
| TBCK | −1.802592091 | 0.00036698 | 0.00956796 | 10.19409643 | 3.7038319 |
| ARFGAP2 | −1.05571895 | 0.00036939 | 0.00960914 | 99.27014286 | 55.69149165 |
| VMA21 | 1.296350724 | 0.00038667 | 0.00999153 | 14.90407952 | 47.51696 |
| BEX2 | −1.953762446 | 0.0003889 | 0.01002659 | 42.92123476 | 16.633287 |
| C20orf194 | 1.79920571 | 0.0003994 | 0.01027462 | 3.980905681 | 14.94492252 |
| RAB29 | −1.369925477 | 0.00040951 | 0.01048821 | 88.32204762 | 50.2578652 |
| NASP | 1.310206027 | 0.00041112 | 0.01050624 | 30.34420248 | 71.02776 |
| C1QTNF6 | 1.673867441 | 0.00041528 | 0.01058907 | 5.089702905 | 18.4102024 |
| PDE7A | 1.250028815 | 0.00041853 | 0.01062531 | 13.26747514 | 34.9504633 |
| SLC31A2 | −1.775058927 | 0.00042405 | 0.01071861 | 216.1743924 | 43.5336665 |
| KLHL2 | 2.248561156 | 0.00042573 | 0.01073759 | 4.456770238 | 16.28283281 |
| FERMT3 | −1.204568177 | 0.00042945 | 0.01080807 | 223.1386952 | 105.6600795 |
| WRNIP1 | 2.312410653 | 0.00043286 | 0.01082584 | 4.016170619 | 36.14411837 |
| PPP1R15B | 1.531191906 | 0.00043296 | 0.01082584 | 13.55361243 | 41.80690205 |
| DENND2D | −1.230933468 | 0.00043207 | 0.01082584 | 177.521881 | 107.0518394 |
| PDS5A | 1.494586311 | 0.00044007 | 0.01097996 | 14.22353581 | 43.45307149 |
| SIGLEC9 | −2.390836951 | 0.00044441 | 0.01106438 | 15.99866633 | 4.49902325 |
| LOC101927482 | 2.304003046 | 0.00045298 | 0.0111772 | 8.79705 | 70.4761062 |
| MBOAT2 | 1.345839205 | 0.00045237 | 0.0111772 | 2.96217119 | 9.6344485 |
| ATF6B | −1.566818376 | 0.00045322 | 0.0111772 | 17.59367905 | 7.97181365 |
| PATL2 | −1.642281469 | 0.00045186 | 0.0111772 | 104.1426341 | 31.97329445 |
| GRN | −1.063273506 | 0.00045971 | 0.01129978 | 791.2872381 | 264.725843 |
| ERI1 | −1.242477967 | 0.00046365 | 0.01134862 | 19.76817686 | 16.5382545 |
| NCALD | −1.68205333 | 0.0004633 | 0.01134862 | 33.27944905 | 17.4302691 |
| FCGR2B | −2.254062836 | 0.00046724 | 0.0114124 | 12.38491424 | 1.09008495 |
| KIR2DS4 | −2.340829735 | 0.00047309 | 0.01153119 | 26.8382171 | 3.872648 |
| HAUS2 | 1.244208464 | 0.00048082 | 0.01169486 | 16.20686571 | 35.1828865 |
| RALGAPB | 1.085068861 | 0.00048941 | 0.01187895 | 8.581074286 | 19.9329055 |
| MAP4 | 1.567107719 | 0.00049617 | 0.01201785 | 23.17886282 | 75.32267281 |
| NPIPB4 | 1.612741663 | 0.00050141 | 0.01206927 | 1.314467681 | 3.3028621 |
| ZBTB9 | −2.327534581 | 0.00049936 | 0.01206927 | 9.2692554 | 4.62615606 |
| CASS4 | 1.232728611 | 0.00050479 | 0.0121004 | 10.33679177 | 23.91917485 |
| MED19 | −1.434707891 | 0.00050469 | 0.0121004 | 44.44179571 | 28.0865765 |
| IRF4 | 1.655714921 | 0.00050764 | 0.01212763 | 8.881926324 | 37.019321 |
| MYO1F | −1.066903485 | 0.00051053 | 0.01216259 | 124.5430429 | 64.6513293 |
| RRN3P2 | −1.714465278 | 0.00051396 | 0.01221926 | 9.252999857 | 5.3255443 |
| FAM134B | 1.638213865 | 0.00052287 | 0.01238031 | 15.5642671 | 40.45138445 |
| POLR2C | −1.501187058 | 0.00052867 | 0.01249202 | 101.9659667 | 54.6541517 |
| CYTH1 | −1.0767005 | 0.0005339 | 0.01258999 | 220.3660476 | 123.9829496 |
| NFYC-AS1 | −1.747707738 | 0.00053774 | 0.01265488 | 7.052072429 | 4.10523765 |
| FAM101B | 1.746294114 | 0.00054483 | 0.01279574 | 11.1097819 | 46.8396593 |
| NFIA | −1.895142906 | 0.00054804 | 0.01282844 | 6.960684619 | 3.316372105 |
| DTX3 | −1.965784578 | 0.00054843 | 0.01282844 | 11.36799743 | 4.4052707 |
| NCBP2-AS2 | −1.27324026 | 0.0005636 | 0.01310398 | 125.8263524 | 62.2609764 |
| C2orf74 | −1.605791569 | 0.0005628 | 0.01310398 | 47.69721333 | 15.21258005 |
| EXOC8 | 1.393473812 | 0.00057299 | 0.01323641 | 5.879713952 | 14.19818165 |
| HIPK1 | 1.259329438 | 0.00057386 | 0.01323641 | 10.37371019 | 29.54598748 |
| PTRHD1 | −1.524952775 | 0.00059245 | 0.01361117 | 112.4339619 | 51.295813 |
| SATB1 | −1.450814725 | 0.00059441 | 0.01362909 | 29.0269319 | 14.87791935 |
| PIGW | −1.70243522 | 0.00059856 | 0.01367036 | 22.70485962 | 9.56260825 |
| ARMT1 | −2.138001294 | 0.00059855 | 0.01367036 | 30.45736219 | 12.96236292 |
| CD96 | 1.082135365 | 0.00060674 | 0.01382991 | 190.6091374 | 445.927872 |
| FAM102A | 1.645022975 | 0.00061089 | 0.01384277 | 15.8821847 | 44.713496 |
| TESPA1 | −1.224535174 | 0.0006107 | 0.01384277 | 34.09646505 | 15.9437695 |
| HHLA3 | −1.783287692 | 0.00061015 | 0.01384277 | 15.31946967 | 19.470709 |
| PHACTR2 | −1.249051197 | 0.00061944 | 0.01400934 | 20.96662857 | 9.260568 |
| UBE2Q2P1 | 1.068947583 | 0.0006375 | 0.0143339 | 8.930663857 | 20.2732585 |
| MDFIC | 1.551874807 | 0.00064418 | 0.014445 | 14.88952865 | 55.02251445 |
| EAF1 | −1.299453137 | 0.00064493 | 0.014445 | 43.09910952 | 24.90341265 |
| FAM102B | 2.109586468 | 0.00064845 | 0.0144927 | 2.63702719 | 27.78515825 |
| CDIPT | −1.217251575 | 0.00064956 | 0.0144927 | 91.82493667 | 48.5642925 |
| PPP3R1 | 1.876147494 | 0.00065664 | 0.01462255 | 6.446798633 | 21.37249 |
| SACS | 2.076350394 | 0.00067552 | 0.01498534 | 2.101448033 | 12.0727165 |
| TSC1 | −1.54806369 | 0.00067936 | 0.0150419 | 12.64261554 | 5.67724477 |
| PPME1 | −1.827792346 | 0.00069454 | 0.01534862 | 39.13166571 | 21.647448 |
| MAPKAPK2 | 1.96713612 | 0.00071533 | 0.01565905 | 0.816921471 | 4.38154145 |
| PCMTD2 | −1.425377237 | 0.00071735 | 0.01567368 | 54.46934619 | 27.86172965 |
| LINC00116 | −1.556841189 | 0.00072237 | 0.01575373 | 27.26614214 | 11.7182429 |
| ACSL4 | 1.827216948 | 0.00072841 | 0.01585224 | 12.69324183 | 30.4276168 |
| PI4KA | 1.676670157 | 0.00073035 | 0.01585224 | 4.408701 | 18.24665 |
| HOPX | 1.210396178 | 0.00073396 | 0.01587589 | 204.3492705 | 494.9027762 |
| LYAR | −1.051479502 | 0.00073619 | 0.01587589 | 162.0467619 | 92.5870975 |
| ELMO2 | −1.107488358 | 0.00073552 | 0.01587589 | 106.4386476 | 49.46063745 |
| DENND1A | 1.80462059 | 0.0007407 | 0.01594357 | 6.1231801 | 23.68573804 |
| CYB561 | −1.489223981 | 0.0007513 | 0.01611206 | 17.6988591 | 8.5005074 |
| TMIGD2 | 1.252962855 | 0.00076016 | 0.01623275 | 14.96517143 | 31.062332 |
| NQO2 | −1.352493557 | 0.00076113 | 0.01623275 | 60.58205 | 25.7614949 |
| SKI | 1.947359558 | 0.00077423 | 0.01645171 | 0.392191043 | 3.10283384 |
| RAB21 | 1.304362685 | 0.00079042 | 0.01673444 | 23.18509605 | 58.62239915 |
| CSF3R | −1.944066722 | 0.00078996 | 0.01673444 | 23.72629945 | 5.661690165 |
| PCNXL3 | 1.820209186 | 0.00079838 | 0.01687221 | 3.427240362 | 13.71444475 |
| CARD8-AS1 | −1.224524067 | 0.00080413 | 0.01696266 | 19.04523843 | 11.228074 |
| MAT2A | 1.005887405 | 0.0008242 | 0.01735462 | 89.89590476 | 154.450275 |
| TADA2B | 1.222867145 | 0.00083479 | 0.0175458 | 4.002363048 | 9.47839305 |
| STX3 | 1.499915799 | 0.00084387 | 0.01767262 | 11.63656086 | 22.42890895 |
| JMY | 1.914684894 | 0.0008624 | 0.0178058 | 2.233548633 | 9.213031 |
| BTBD7 | 1.723887579 | 0.00085229 | 0.0178058 | 3.343328867 | 11.70509428 |
| RSBN1L | −1.459929578 | 0.00085752 | 0.0178058 | 8.030019667 | 3.9945429 |
| CIAPIN1 | −1.565209613 | 0.00085533 | 0.0178058 | 77.74407619 | 41.0017459 |
| DOK1 | −1.768374315 | 0.00085583 | 0.0178058 | 37.27138333 | 13.1786472 |
| DCP1B | −2.073917448 | 0.00086078 | 0.0178058 | 37.24625476 | 12.0182602 |
| MANBA | −1.808919441 | 0.00089272 | 0.01836417 | 18.8245423 | 7.538196335 |
| MTSS1 | −1.761070691 | 0.0008946 | 0.01837015 | 18.5287793 | 6.52629872 |
| RPS2 | 1.110924704 | 0.00090508 | 0.01855263 | 1951.231333 | 3025.4425 |
| ITGA5 | −1.332979911 | 0.00091178 | 0.01865696 | 62.5509 | 26.01677665 |
| ARID3B | 1.475041207 | 0.00091604 | 0.01867827 | 13.15811477 | 46.9491545 |
| CD300A | −1.832905746 | 0.00092368 | 0.01880092 | 113.4101663 | 39.52995785 |
| POLR3F | −2.271732688 | 0.00093671 | 0.01899954 | 17.56640771 | 5.199307 |
| MXD4 | 1.476173375 | 0.00095074 | 0.01918342 | 9.359247619 | 27.3796455 |
| CPNE8 | −1.877032265 | 0.00097453 | 0.01962939 | 15.75364538 | 5.43907834 |
| NCF4 | 1.357098972 | 0.00097951 | 0.01966147 | 56.53410076 | 86.755054 |
| DUS3L | −1.484396682 | 0.00097858 | 0.01966147 | 36.38678795 | 14.53410265 |
| CDC26 | −1.173174999 | 0.00100179 | 0.02007394 | 238.7674286 | 119.7321551 |
| TGFBR3 | −1.435187571 | 0.00100384 | 0.02008029 | 67.24751205 | 29.94398345 |
| HDDC2 | −1.268862316 | 0.00101088 | 0.02018626 | 123.8036667 | 67.9808733 |
| SARDH | 2.294096038 | 0.00102414 | 0.02034607 | 3.728724762 | 20.16373173 |
| NMRAL1 | −1.475564497 | 0.00102413 | 0.02034607 | 101.2016619 | 42.4074745 |
| DDX49 | −1.679761061 | 0.00102414 | 0.02034607 | 32.13191195 | 11.88200645 |
| GOLGA8B | 1.128453104 | 0.00103932 | 0.02061237 | 7.629956238 | 17.656008 |
| APMAP | −1.026634309 | 0.00106369 | 0.02105967 | 533.4089048 | 258.1887565 |
| SPSB3 | −1.105088531 | 0.00106718 | 0.02109281 | 217.7387857 | 123.0655227 |
| ETV1 | 2.434846563 | 0.00107798 | 0.02123713 | 0.310453333 | 9.36143927 |
| TNFSF14 | 1.050001711 | 0.00108212 | 0.02123713 | 22.60123595 | 60.4481919 |
| RANBP3 | −1.327147463 | 0.00108128 | 0.02123713 | 38.6904619 | 14.96831215 |
| ATP10D | 1.855742812 | 0.00109212 | 0.02133783 | 4.340767567 | 19.78619264 |
| LOC102606465 | −2.447368854 | 0.00109246 | 0.02133783 | 14.16471976 | 0.27695965 |
| TTN | 1.460996679 | 0.00110171 | 0.0214824 | 0.51753301 | 1.68269195 |
| TRAF3IP2 | 1.097128158 | 0.00111682 | 0.02170402 | 4.3813 | 10.520044 |
| HEXA | −1.214689315 | 0.00112365 | 0.02176397 | 190.0591476 | 100.2384131 |
| FRY | −2.380982116 | 0.00112832 | 0.02180454 | 1.7706065 | 0.08596388 |
| BID | −1.335634005 | 0.00115289 | 0.02218219 | 80.79564286 | 33.02446055 |
| AGAP2 | 1.25933984 | 0.00116449 | 0.02236824 | 12.47762385 | 25.56242397 |
| SLAMF7 | −1.097981708 | 0.00118377 | 0.02270106 | 154.3703411 | 97.756458 |
| GPX3 | −2.009859501 | 0.0011912 | 0.0228058 | 68.71822389 | 10.9501879 |
| INPP5F | 2.094388653 | 0.00119747 | 0.02285048 | 0.683434986 | 6.52550963 |
| PTMS | 1.527260067 | 0.00119677 | 0.02285048 | 14.55434505 | 39.8358905 |
| ATF4 | 1.13820403 | 0.00126634 | 0.02400673 | 66.5021559 | 132.054105 |
| RSU1 | −1.034060739 | 0.00128444 | 0.02427042 | 113.3402333 | 76.4465641 |
| ICAM1 | 1.307628761 | 0.00128715 | 0.02428207 | 45.27852671 | 106.998965 |
| GNAI2 | 1.431878899 | 0.00129323 | 0.02431769 | 77.9547819 | 225.6534442 |
| RFX7 | 1.493182438 | 0.00130679 | 0.02453303 | 9.145289048 | 19.7635245 |
| AFAP1L2 | 2.217430641 | 0.00131729 | 0.02461261 | 0.917768462 | 5.888400985 |
| STX11 | −1.074261581 | 0.0013174 | 0.02461261 | 45.60824286 | 24.79838315 |
| CEP78 | −1.731280746 | 0.00131509 | 0.02461261 | 62.41426857 | 25.59219025 |
| SERGEF | −1.628390874 | 0.00132889 | 0.02474767 | 51.05715719 | 27.04479875 |
| KLHDC3 | −1.485905935 | 0.00135796 | 0.02516789 | 105.7995905 | 44.6288617 |
| MCU | −1.781678671 | 0.00135587 | 0.02516789 | 21.059248 | 7.2983622 |
| ZNF43 | 1.365362673 | 0.00136523 | 0.02526219 | 5.320740857 | 19.68480875 |
| STT3B | 1.445687084 | 0.00137371 | 0.02528219 | 50.40380312 | 108.1294734 |
| ZGPAT | −1.2667066 | 0.00136929 | 0.02528219 | 63.04500952 | 31.116643 |
| BINI | −1.286241264 | 0.00137503 | 0.02528219 | 115.9730212 | 47.48353135 |
| PRAF2 | 1.088429664 | 0.00139187 | 0.02547835 | 52.76120524 | 130.40493 |
| FBXL15 | −1.245548804 | 0.00139228 | 0.02547835 | 34.4780081 | 14.03420705 |
| CMKLR1 | −2.393657255 | 0.0013904 | 0.02547835 | 7.379469857 | 0.01679331 |
| PROS1 | −2.381361313 | 0.00140406 | 0.02561766 | 14.30501987 | 0.271954765 |
| UBAP2L | −1.107731449 | 0.00142117 | 0.02584397 | 60.0890381 | 32.03060748 |
| ILF2 | −1.322111404 | 0.00144866 | 0.02626157 | 268.6788571 | 134.0617756 |
| SOS1 | 1.916563335 | 0.00146636 | 0.02649961 | 3.472780429 | 14.67357545 |
| CLU | 1.445639436 | 0.00147363 | 0.02658954 | 33.64088286 | 70.5663326 |
| DENND1B | 1.31053697 | 0.00148785 | 0.02680441 | 8.868308262 | 27.4410549 |
| CCDC137 | −1.424536409 | 0.00149865 | 0.02695706 | 45.84479614 | 26.23133495 |
| COMMD1 | −1.163651607 | 0.00151914 | 0.02728341 | 155.913578 | 80.0766678 |
| ECT2 | 1.625708892 | 0.00153602 | 0.02754382 | 1.838423486 | 6.2113756 |
| TNFRSF4 | −1.818299294 | 0.00154166 | 0.02760233 | 21.45005543 | 5.2371664 |
| ERBB2 | −2.074275535 | 0.00154754 | 0.02766499 | 4.287361633 | 0.655102315 |
| PTGDS | −2.288741647 | 0.00155432 | 0.02770077 | 28.53224938 | 4.8148675 |
| GZMM | −1.082018373 | 0.00155738 | 0.02771274 | 283.5499905 | 139.63146 |
| UBE2E2 | −2.05240565 | 0.00158064 | 0.02804073 | 20.36653043 | 15.0806748 |
| RMDN1 | −1.415147467 | 0.0015921 | 0.02820087 | 36.57705833 | 15.18240745 |
| CTDNEP1 | 1.714000117 | 0.00160704 | 0.02842206 | 16.60610719 | 63.6246042 |
| CDC42SE1 | −1.188507354 | 0.00161548 | 0.0285278 | 224.792919 | 115.0242707 |
| IPP | −1.320206248 | 0.00164409 | 0.02894493 | 18.17084054 | 11.66533725 |
| CDKN2D | −1.36879016 | 0.00166031 | 0.02909798 | 96.05093448 | 58.9614505 |
| ATP1B1 | 1.283027758 | 0.00167718 | 0.02914164 | 52.0284119 | 75.36316685 |
| UBR4 | 1.08473922 | 0.00167246 | 0.02914164 | 4.653459695 | 11.783862 |
| KIR3DX1 | −1.049750649 | 0.00166595 | 0.02914164 | 4.466350167 | 1.73610435 |
| OXLD1 | −1.420013153 | 0.00167581 | 0.02914164 | 60.42109286 | 34.3730691 |
| WDR37 | −1.670567502 | 0.00167227 | 0.02914164 | 23.3282819 | 9.586654835 |
| PPOX | −1.109942601 | 0.00169085 | 0.02932322 | 56.83536667 | 22.04271505 |
| TGFB1 | 1.171557058 | 0.00169425 | 0.02933837 | 34.42022667 | 69.536372 |
| ZNF557 | −1.236244688 | 0.00170752 | 0.02952401 | 20.62738043 | 9.55651765 |
| ULK2 | 1.325400012 | 0.00171981 | 0.02969224 | 1.888480762 | 4.9796416 |
| CYP2U1 | −2.049943251 | 0.00174364 | 0.03005893 | 11.44327268 | 5.70043324 |
| NR3C1 | 1.031848789 | 0.00175779 | 0.0302579 | 38.35911238 | 94.7195966 |
| SLC27A2 | 1.98883201 | 0.00181163 | 0.03090959 | 7.761453905 | 26.6987376 |
| TFPT | −1.502632807 | 0.00182073 | 0.03101928 | 30.40861905 | 12.54043605 |
| MKI67 | 2.213790722 | 0.00182667 | 0.03107475 | 0.396551648 | 2.29143949 |
| CST3 | −1.160291651 | 0.00184011 | 0.03125756 | 137.9140714 | 67.30464325 |
| LSR | 1.60329948 | 0.00187247 | 0.0316448 | 21.59478005 | 55.67163075 |
| DPP4 | 1.299281837 | 0.00187217 | 0.0316448 | 30.0033817 | 77.8169133 |
| CASP1 | −1.156469494 | 0.00187381 | 0.0316448 | 205.0463841 | 99.98735945 |
| AHSA2 | 1.382667831 | 0.00187723 | 0.03165638 | 14.3165809 | 35.795771 |
| TARS2 | −1.214923276 | 0.00191337 | 0.0322191 | 45.92252857 | 26.3175739 |
| ZDHHC11 | 2.168394746 | 0.00192446 | 0.0323588 | 2.041777762 | 22.7649697 |
| ZNF10 | 1.777188919 | 0.00192914 | 0.03239061 | 3.195804 | 10.83783919 |
| IGSF6 | −1.844095235 | 0.00195275 | 0.03269235 | 106.8541425 | 21.85815275 |
| FAM208A | 1.049249105 | 0.00195718 | 0.03271935 | 14.16893262 | 34.97957965 |
| MTMR9 | 1.315310954 | 0.00196674 | 0.03281718 | 5.720141533 | 16.17714945 |
| CSRP2BP | −1.82037327 | 0.00197683 | 0.0329056 | 16.6018126 | 8.3935547 |
| PASK | −1.686985134 | 0.00198289 | 0.03295917 | 14.93246762 | 7.15466456 |
| MKKS | −1.437049717 | 0.00200493 | 0.03327787 | 38.0899919 | 17.0888044 |
| FAM46A | −1.81198712 | 0.00202033 | 0.03348561 | 11.81714486 | 5.579773015 |
| ITFG3 | −1.702008908 | 0.00202437 | 0.03350458 | 20.83019762 | 11.13164403 |
| DLG5 | −1.953834863 | 0.00203104 | 0.03356717 | 3.435019381 | 0.903906625 |
| KIAA0101 | 1.444676814 | 0.00204015 | 0.03365834 | 6.675146857 | 25.12704715 |
| SAAL1 | −1.583018596 | 0.00204236 | 0.03365834 | 51.56066124 | 17.6734762 |
| MMP25 | 1.459299708 | 0.00206447 | 0.03383054 | 2.632463762 | 7.9999775 |
| TATDN3 | −1.074764039 | 0.0020589 | 0.03383054 | 28.73702095 | 12.906969 |
| LOC100996447 | −1.156694083 | 0.00206329 | 0.03383054 | 42.42375095 | 23.50972975 |
| NIFK-AS1 | −2.019056074 | 0.00205963 | 0.03383054 | 11.83977338 | 3.4095151 |
| XPR1 | 1.392572867 | 0.00207024 | 0.03387718 | 3.614010729 | 9.8424576 |
| C11orf21 | −1.545286707 | 0.00208323 | 0.03404167 | 43.01835476 | 16.9673255 |
| ICOS | 1.37194015 | 0.00213471 | 0.03468756 | 52.45500238 | 122.4600494 |
| TULP4 | −1.370180377 | 0.00213448 | 0.03468756 | 11.6970605 | 6.69757389 |
| INPP4A | 1.32469001 | 0.00214188 | 0.03475543 | 11.57118199 | 28.38812305 |
| DFNB31 | 1.739227223 | 0.00216261 | 0.03499379 | 3.190885052 | 11.18772015 |
| ANKDD1A | 1.666867831 | 0.00216252 | 0.03499379 | 2.474719238 | 7.0630617 |
| PTPRJ | 1.045068081 | 0.00216996 | 0.03506395 | 7.210654619 | 22.43940385 |
| DST | 1.781307402 | 0.00218105 | 0.03519407 | 0.740807522 | 2.481083109 |
| SMIM3 | 1.701657926 | 0.00219219 | 0.03522757 | 22.12527529 | 101.0869653 |
| MARK2 | 1.627042544 | 0.00219035 | 0.03522757 | 3.95061229 | 13.006327 |
| LPAR5 | −1.432358065 | 0.00219223 | 0.03522757 | 37.58498552 | 22.93229028 |
| PRMT5 | −1.713226626 | 0.00220091 | 0.03531809 | 23.97953357 | 9.213471305 |
| DMKN | −2.116833951 | 0.00220939 | 0.03540511 | 7.914099052 | 1.584939 |
| PIGL | 1.533178933 | 0.00222549 | 0.03561394 | 17.87966267 | 48.98544735 |
| CXorf23 | 1.379998376 | 0.00224041 | 0.03580339 | 2.371735467 | 14.63350675 |
| ERLIN1 | −1.898071983 | 0.00225202 | 0.0358865 | 20.30472526 | 6.76962096 |
| ZNF689 | −2.141277674 | 0.00225575 | 0.0358865 | 9.676069048 | 2.30918672 |
| UQCC3 | −1.448663386 | 0.00227194 | 0.0360096 | 12.19188619 | 5.86697545 |
| ZNF587B | −1.482821582 | 0.00228038 | 0.03609416 | 14.89556113 | 7.10224025 |
| ENPP5 | −1.963798982 | 0.00229414 | 0.03621307 | 19.36512957 | 9.023781 |
| MRC1 | −1.322294944 | 0.00233316 | 0.03662963 | 52.31267884 | 15.67313203 |
| HDAC6 | −1.449599247 | 0.00233315 | 0.03662963 | 28.30981905 | 11.9235349 |
| CEP63 | −1.646464036 | 0.00233294 | 0.03662963 | 12.22384563 | 5.27964766 |
| CREM | 1.063360811 | 0.00234737 | 0.03670373 | 42.30688857 | 71.516919 |
| SYNE1 | −1.009975398 | 0.00234287 | 0.03670373 | 16.10141843 | 10.3669361 |
| SVIL | −1.361066127 | 0.00234573 | 0.03670373 | 9.580544286 | 4.22403719 |
| NME4 | −1.694305479 | 0.00235394 | 0.03675689 | 15.39367581 | 6.5277521 |
| ZNF48 | 2.001817403 | 0.00235989 | 0.03680028 | 0.95379711 | 2.93424308 |
| HIATL2 | 1.639605506 | 0.00237954 | 0.03691362 | 11.04763529 | 30.088932 |
| TNFRSF10A | 1.233874024 | 0.00237704 | 0.03691362 | 39.42923329 | 87.286662 |
| PAG1 | 1.142151432 | 0.00237849 | 0.03691362 | 13.7221171 | 36.40958485 |
| MELK | 1.140922968 | 0.00237988 | 0.03691362 | 2.926656905 | 9.4487255 |
| AGAP6 | −1.615980385 | 0.0023937 | 0.03702892 | 4.65957 | 4.15214054 |
| DDA1 | −1.010469768 | 0.00241255 | 0.03727088 | 18.96847857 | 13.5441625 |
| ARFRP1 | −1.283380491 | 0.00243869 | 0.03762465 | 46.60115286 | 22.098322 |
| RCOR1 | 1.944709033 | 0.00244246 | 0.03763275 | 0.534440586 | 4.51260244 |
| PDK2 | −1.664734422 | 0.00246036 | 0.03770812 | 29.69540175 | 11.07580269 |
| FAM216A | −1.983848294 | 0.0024698 | 0.03780297 | 13.79144952 | 6.5779684 |
| VAMP5 | −1.136383361 | 0.00247878 | 0.03789039 | 158.8593286 | 84.24594405 |
| HERC1 | 1.091641582 | 0.00253247 | 0.03866021 | 9.368462695 | 26.80007538 |
| ZNF555 | −1.181944735 | 0.00253582 | 0.03866035 | 3.489925914 | 1.97726846 |
| UBE2S | 1.605973396 | 0.00254412 | 0.03873612 | 52.33753024 | 129.0238405 |
| CCDC28B | −2.032747097 | 0.00257249 | 0.03901441 | 13.39630476 | 2.8255758 |
| TRA2B | 1.071850101 | 0.00258234 | 0.03906167 | 34.72536619 | 75.910455 |
| DERA | −1.577728539 | 0.00257957 | 0.03906167 | 43.97195548 | 20.5084549 |
| TSPYL2 | −1.053848144 | 0.00260025 | 0.03923037 | 91.85230476 | 58.63886275 |
| PKIG | −2.077214187 | 0.002643 | 0.03977177 | 10.41545852 | 1.8342887 |
| CCAT1 | 1.445071665 | 0.0026754 | 0.04006706 | 0.528602548 | 16.95488595 |
| C1orf35 | −1.378296383 | 0.0026703 | 0.04006706 | 39.94078238 | 24.80257585 |
| CDKN1B | 1.282793989 | 0.00272467 | 0.04068418 | 37.4884511 | 115.6148839 |
| CISD3 | −1.10729904 | 0.00274379 | 0.04091698 | 43.8215181 | 21.41737 |
| CKS2 | 1.378226136 | 0.00275569 | 0.04092812 | 44.10446333 | 217.9010409 |
| IL4R | 1.173407546 | 0.00275832 | 0.04092812 | 20.8707369 | 44.223466 |
| ANGPT2 | 1.090592385 | 0.0027657 | 0.04092812 | 1.214743286 | 4.68820725 |
| SEC24C | −1.065187919 | 0.00275712 | 0.04092812 | 78.9841681 | 45.24677965 |
| VIPR1 | −2.245159841 | 0.00276548 | 0.04092812 | 7.878956714 | 0.078206365 |
| MED14 | 1.56112954 | 0.00281212 | 0.04150917 | 4.157886333 | 12.40530445 |
| AHI1 | 1.317518043 | 0.00281763 | 0.04153763 | 8.087297457 | 23.78564948 |
| DPH6 | −2.003375054 | 0.00282831 | 0.04164227 | 9.295661143 | 3.259904245 |
| EHD1 | −1.001672515 | 0.00283359 | 0.04166709 | 59.14083405 | 36.4075224 |
| PSPH | −1.152155764 | 0.00284639 | 0.04180226 | 10.84557571 | 8.5721058 |
| SH3GLB2 | 1.512043303 | 0.00285527 | 0.04187964 | 6.430948286 | 19.3427 |
| ARHGAP26 | 1.202808995 | 0.00290391 | 0.04237881 | 4.392610524 | 14.80365015 |
| HIF1A | 1.052731671 | 0.00290373 | 0.04237881 | 38.03787619 | 90.85415895 |
| MYO9B | 1.008632072 | 0.00290113 | 0.04237881 | 8.362505238 | 25.050393 |
| MUS81 | −1.134364426 | 0.00289302 | 0.04237881 | 28.52751952 | 18.2275496 |
| HKR1 | −1.831633144 | 0.00292061 | 0.04250344 | 22.39177522 | 8.01066489 |
| PTAR1 | 1.699146857 | 0.00294713 | 0.04268765 | 2.743866857 | 8.368398865 |
| TMEM187 | −1.825720333 | 0.00294715 | 0.04268765 | 22.12027881 | 7.65957825 |
| CLK3 | −1.001182295 | 0.00298754 | 0.04316491 | 116.5983771 | 64.7280973 |
| EXOC6 | 1.462440669 | 0.00301764 | 0.0434915 | 18.62345299 | 52.863598 |
| ETFA | −1.033853587 | 0.00308391 | 0.04439151 | 211.4412429 | 133.8260265 |
| RDH13 | −1.827515919 | 0.00310699 | 0.04466828 | 13.62577429 | 4.675346 |
| MTERF1 | −1.58817958 | 0.00311175 | 0.04468131 | 27.75623086 | 14.80039655 |
| LOC101927027 | 1.573304325 | 0.00312364 | 0.04474127 | 4.211369714 | 26.13527023 |
| CNP | −1.097403536 | 0.00313197 | 0.04480529 | 24.98781305 | 14.29346375 |
| ARFGEF2 | 1.241620144 | 0.00313644 | 0.04481405 | 5.907125638 | 15.1121541 |
| MCM3AP | 1.160487177 | 0.00316806 | 0.04509921 | 18.98804667 | 42.63157293 |
| SFPQ | 1.206824864 | 0.00318158 | 0.04523452 | 47.61741619 | 88.602034 |
| RAD9A | −1.374057685 | 0.00318536 | 0.04523452 | 8.711004286 | 4.91682275 |
| DAXX | −1.06083514 | 0.00319593 | 0.04527372 | 30.36822905 | 19.98259299 |
| LOC100996286 | −1.83607122 | 0.00319235 | 0.04527372 | 60.76818519 | 17.25592475 |
| TMEM237 | −1.951065251 | 0.00328224 | 0.04630322 | 4.673383062 | 4.04188854 |
| ARID1A | 1.387569106 | 0.00331076 | 0.0463145 | 9.368136124 | 34.768239 |
| CPT2 | −1.139351315 | 0.00329723 | 0.0463145 | 30.83033438 | 21.19235005 |
| ABT1 | −1.19573184 | 0.00331435 | 0.0463145 | 132.1675667 | 74.13886765 |
| TXK | −1.569311232 | 0.00331402 | 0.0463145 | 43.40230785 | 18.92011706 |
| LRRC45 | −1.891079088 | 0.00329804 | 0.0463145 | 8.30033181 | 3.13344467 |
| ZNF280C | 1.36949936 | 0.00332362 | 0.04634687 | 2.081992714 | 10.13766255 |
| MAP2K4 | 1.31117468 | 0.00333665 | 0.04636151 | 26.25060724 | 72.47427745 |
| TUBB | 1.097554669 | 0.0033353 | 0.04636151 | 17.42295328 | 33.04663 |
| PLEKHA1 | −1.045263518 | 0.003329 | 0.04636151 | 54.94448481 | 32.1948511 |
| BBS5 | 1.096834176 | 0.0033706 | 0.04666548 | 3.122230367 | 9.2493915 |
| GDAP2 | 1.290313972 | 0.0034067 | 0.04705306 | 6.706050762 | 13.2542057 |
| AHR | 1.102241377 | 0.00342225 | 0.04715557 | 7.888413095 | 13.6936725 |
| TNFRSF9 | −1.198960983 | 0.00342212 | 0.04715557 | 13.75290395 | 7.85170705 |
| MCOLN2 | 1.245703069 | 0.00346294 | 0.04760314 | 21.82523003 | 47.5998025 |
| XYLT1 | 1.99585507 | 0.00348152 | 0.04780194 | 0.782486952 | 3.604668105 |
| PLCG2 | −1.227346251 | 0.00350186 | 0.04797513 | 11.47080835 | 4.67658275 |
| PRKAB1 | −1.348576658 | 0.00350273 | 0.04797513 | 58.58896667 | 38.1610866 |
| TGFBR1 | 1.130381688 | 0.00353755 | 0.04825283 | 17.15331511 | 33.0276235 |
| U2AF1L4 | −1.053266078 | 0.00353573 | 0.04825283 | 72.834 | 42.5640561 |
| SNHG7 | 1.243507546 | 0.00355025 | 0.04834502 | 4.981000238 | 18.98669895 |
| PPIL4 | 1.13138956 | 0.00361378 | 0.04915252 | 53.22107343 | 109.675935 |
| NEK8 | −1.427122553 | 0.00362368 | 0.04917181 | 5.678052524 | 2.17769965 |
| PIK3R2 | −1.504052614 | 0.0036227 | 0.04917181 | 4.701518429 | 1.342299575 |
| MRPS18B | −1.160658918 | 0.00364157 | 0.04934871 | 19.64079799 | 9.98160735 |
| ABHD17B | −1.180701066 | 0.00365373 | 0.04934871 | 12.83117357 | 8.93513765 |
| ZFAND1 | −1.35854761 | 0.00365182 | 0.04934871 | 50.26811962 | 28.80094995 |
| TNFAIP8L2 | −1.853922479 | 0.00369231 | 0.049754 | 57.96041905 | 14.49336125 |

TABLE 4
List of genes differentially expressed between Tumor TRM compared to Tumor non-TRM

| | | | | Mean TPM | Mean TPM | Common genes |
|---|---|---|---|---|---|---|
| Gene ID | log2FoldChange | pvalue | padj | Tumor non-TRM | Tumor TRM | with Lung |
| ITGAE | 3.740189693 | 3.12E−72 | 3.85E−68 | 33.0593668 | 407.095 | Yes |
| S1PR5 | −4.523578131 | 7.84E−34 | 4.84E−30 | 61.60472972 | 1.679153684 | Yes |
| GSG2 | 3.635833487 | 2.36E−28 | 7.27E−25 | 0.43646014 | 10.79137868 | Yes |
| GZMB | 1.997567017 | 5.45E−28 | 1.34E−24 | 2123.78152 | 6599.232632 | |
| S1PR1 | −3.027979011 | 9.21E−25 | 1.89E−21 | 191.05454 | 26.11258247 | Yes |
| MYO7A | 3.513567966 | 5.58E−18 | 8.61E−15 | 2.571285816 | 44.23771526 | Yes |
| FOS | −1.067494599 | 5.56E−18 | 8.61E−15 | 2389.31976 | 1583.288684 | |
| GPR25 | 3.483981326 | 7.12E−17 | 9.38E−14 | 4.76494824 | 85.23201053 | Yes |
| PLAC8 | −2.474542579 | 7.61E−17 | 9.38E−14 | 55.3151272 | 10.19808989 | Yes |
| LAYN | 3.621535741 | 1.31E−16 | 1.47E−13 | 1.848024708 | 52.53625789 | |
| KRT86 | 3.381632722 | 4.46E−16 | 4.58E−13 | 3.71512388 | 90.02109042 | Yes |
| STMN1 | 1.366049689 | 7.63E−16 | 6.72E−13 | 85.79118 | 171.0376579 | |
| PDCD1 | 1.57324167 | 1.08E−15 | 8.87E−13 | 86.55487464 | 225.5345789 | Yes |
| RBPJ | 1.633587013 | 1.51E−15 | 1.16E−12 | 68.462808 | 235.5415263 | Yes |
| TCF7 | −1.919812967 | 7.00E−15 | 4.80E−12 | 54.29527692 | 16.48907895 | |
| KLRG1 | −2.072161115 | 6.62E−15 | 4.80E−12 | 487.1466948 | 115.8208 | Yes |
| ENTPD1 | 2.51222904 | 1.22E−14 | 7.94E−12 | 9.068538 | 51.02960526 | |
| ZNF683 | 2.168329213 | 1.38E−14 | 8.50E−12 | 146.606496 | 728.6827368 | Yes |
| SPRY1 | 3.136164239 | 2.23E−14 | 1.31E−11 | 4.932731568 | 55.74590526 | Yes |
| CCR7 | −2.22354341 | 2.48E−14 | 1.39E−11 | 227.195484 | 56.20075605 | Yes |
| KLRC2 | 2.347036307 | 7.93E−14 | 4.08E−11 | 42.90052344 | 170.3632789 | |
| FCGR3A | −2.930611162 | 1.81E−13 | 8.59E−11 | 119.760642 | 14.86318363 | Yes |
| ALOX5AP | 1.211931191 | 3.98E−13 | 1.82E−10 | 709.35508 | 1504.629105 | |
| TOX | 1.04690617 | 2.72E−12 | 1.20E−09 | 52.981492 | 111.2167158 | |
| TNS3 | 2.871610018 | 1.26E−11 | 5.17E−09 | 1.578477292 | 18.24837105 | |
| SRGAP3 | 2.957823113 | 1.36E−11 | 5.42E−09 | 1.467354292 | 21.03388737 | Yes |
| CCDC109B | −1.149921845 | 1.41E−11 | 5.44E−09 | 373.868636 | 168.8402895 | |
| CLNK | 2.981297463 | 1.56E−11 | 5.82E−09 | 3.97429732 | 48.79801947 | Yes |
| AFAP1L2 | 2.730768148 | 2.03E−11 | 7.35E−09 | 10.19192002 | 48.02176947 | Yes |
| SELL | −2.397423991 | 2.35E−11 | 8.05E−09 | 442.230628 | 72.96528086 | Yes |
| GZMK | −1.337577522 | 2.50E−11 | 8.33E−09 | 3386.79804 | 1582.416847 | Yes |
| IL7R | −1.39682464 | 2.89E−11 | 9.39E−09 | 377.3089102 | 176.2078421 | |
| KLRC1 | 2.528735 | 4.60E−11 | 1.42E−08 | 32.23271408 | 201.5704368 | Yes |
| GOLIM4 | 2.325048002 | 5.07E−11 | 1.53E−08 | 22.3738512 | 81.66201579 | Yes |
| CXCL13 | 2.786215532 | 7.81E−11 | 2.15E−08 | 200.4329206 | 1830.654842 | |
| AKAP5 | 2.119777167 | 7.69E−11 | 2.15E−08 | 3.498434 | 12.78739474 | |
| HAVCR2 | 2.115630958 | 8.47E−11 | 2.22E−08 | 71.16694344 | 334.6920737 | |
| RASSF3 | −1.294121912 | 1.08E−10 | 2.77E−08 | 88.174812 | 37.24806632 | |
| KIR2DL4 | 2.739112566 | 1.92E−10 | 4.82E−08 | 6.891746048 | 90.73159421 | |
| CD63 | 1.060494213 | 2.91E−10 | 7.19E−08 | 305.35808 | 549.2277895 | |
| PHLDA1 | 1.92802647 | 3.62E−10 | 8.76E−08 | 20.7194988 | 72.30838947 | Yes |
| CHRM3-AS2 | 2.773184838 | 4.89E−10 | 1.16E−07 | 2.12001284 | 45.31885842 | |
| ATP8B4 | 2.747605956 | 5.80E−10 | 1.30E−07 | 1.198385096 | 13.04629837 | Yes |
| TNFRSF9 | 2.018972638 | 6.12E−10 | 1.35E−07 | 17.28012864 | 75.16161053 | |
| CSF1 | 2.169258026 | 1.45E−09 | 3.04E−07 | 18.68793199 | 83.02554737 | |
| FGR | −2.174444347 | 1.89E−09 | 3.88E−07 | 81.8702808 | 11.86666842 | Yes |
| RAB27A | 1.270864982 | 2.44E−09 | 4.85E−07 | 72.8336226 | 165.1855579 | |
| FLOT1 | −1.198609437 | 2.42E−09 | 4.85E−07 | 19.8708108 | 9.655719 | |
| PLEK | −1.920067488 | 2.66E−09 | 5.21E−07 | 189.283604 | 41.99313458 | Yes |
| KLF3 | −2.373844864 | 2.87E−09 | 5.54E−07 | 25.2746158 | 4.824050537 | Yes |
| DAPK2 | 2.020364656 | 3.05E−09 | 5.79E−07 | 15.41775706 | 64.87444211 | Yes |
| FAM3C | 1.645072966 | 3.50E−09 | 6.55E−07 | 33.074066 | 118.3906789 | |
| LINC00861 | −1.200761735 | 3.95E−09 | 7.27E−07 | 131.4451524 | 53.62909526 | |
| ARHGAP11A | 1.788648298 | 4.28E−09 | 7.77E−07 | 3.261430284 | 10.31301842 | Yes |
| RRM2 | 2.032567257 | 4.49E−09 | 8.03E−07 | 24.39809328 | 51.55875105 | |
| ETV1 | 2.431245366 | 7.54E−09 | 1.31E−06 | 1.990125728 | 17.19050579 | Yes |
| CD109 | 2.209733753 | 8.57E−09 | 1.47E−06 | 1.058001288 | 4.155530368 | |
| CD7 | 1.243952111 | 9.59E−09 | 1.62E−06 | 152.706784 | 335.0916316 | |
| DBH-AS1 | 2.446850699 | 1.11E−08 | 1.84E−06 | 1.18816808 | 9.190445 | |
| TMIGD2 | 1.755365521 | 1.23E−08 | 2.02E−06 | 12.8405268 | 46.83354474 | Yes |
| SIRPG | 1.173047291 | 1.38E−08 | 2.24E−06 | 185.4196008 | 477.9031579 | |
| SORL1 | −1.620313508 | 1.63E−08 | 2.54E−06 | 29.9296156 | 11.53147181 | Yes |
| DOCK5 | 2.249256949 | 1.83E−08 | 2.81E−06 | 1.279708636 | 3.712515426 | Yes |
| CXCR6 | 1.209548172 | 2.19E−08 | 3.30E−06 | 639.847916 | 1379.978 | Yes |
| CCL3 | 1.308047955 | 2.27E−08 | 3.37E−06 | 448.815904 | 879.4891053 | |
| FAM65B | −1.285468642 | 2.86E−08 | 4.21E−06 | 67.4624064 | 25.83885526 | Yes |
| KIF2C | 2.340084386 | 3.24E−08 | 4.65E−06 | 2.716626244 | 15.13782305 | |
| CD101 | 2.149991882 | 4.52E−08 | 6.27E−06 | 10.88423144 | 46.95427195 | |
| PZP | −1.943336483 | 5.16E−08 | 6.99E−06 | 11.83152352 | 3.274193368 | Yes |
| PAQR4 | 2.35562189 | 6.71E−08 | 9.00E−06 | 1.689015624 | 10.24887293 | |
| KLRF1 | −2.393123248 | 1.07E−07 | 1.41E−05 | 69.15205336 | 3.035728737 | Yes |
| XCL1 | 1.366128861 | 1.27E−07 | 1.64E−05 | 39.7461872 | 96.09117895 | Yes |
| CAPG | 1.119966807 | 1.59E−07 | 2.03E−05 | 231.99522 | 437.7557895 | Yes |
| WBP4 | 1.209997096 | 1.74E−07 | 2.17E−05 | 29.181684 | 35.85908947 | |
| IVNS1ABP | 1.261438848 | 1.82E−07 | 2.25E−05 | 128.6844636 | 275.7747368 | Yes |
| ADAM19 | 1.007948314 | 2.23E−07 | 2.70E−05 | 23.6940676 | 53.16917895 | Yes |
| DHRS3 | −1.538676923 | 2.94E−07 | 3.52E−05 | 150.522892 | 53.74266979 | |
| CTLA4 | 2.049857964 | 3.17E−07 | 3.76E−05 | 50.97031992 | 243.8928526 | Yes |
| CLIC3 | 1.098704683 | 4.25E−07 | 4.77E−05 | 58.91024892 | 122.7453368 | |
| FCGR3B | −2.089580696 | 4.25E−07 | 4.77E−05 | 15.66373078 | 1.389735158 | |
| CX3CR1 | −2.269155106 | 4.32E−07 | 4.80E−05 | 62.33696111 | 1.737185411 | Yes |
| RASA3 | −1.805687466 | 5.15E−07 | 5.62E−05 | 54.1342792 | 11.73182377 | Yes |
| IFITM10 | 2.259689528 | 5.26E−07 | 5.64E−05 | 1.272724276 | 11.72409947 | |
| C1orf21 | −1.891368092 | 7.09E−07 | 7.41E−05 | 11.4573743 | 2.148275695 | Yes |
| ATM | −1.005296267 | 7.86E−07 | 8.15E−05 | 19.96884244 | 11.57904211 | |
| A2M | −1.606407971 | 9.37E−07 | 9.64E−05 | 46.2580028 | 18.43226479 | Yes |
| GEM | 2.209029285 | 1.15E−06 | 0.000114883 | 2.765744876 | 25.92595789 | |
| RASGRP2 | −1.596008125 | 1.16E−06 | 0.000115288 | 51.92487504 | 14.19080942 | Yes |
| RAD51AP1 | 2.184111701 | 1.17E−06 | 0.00011594 | 2.22739396 | 17.47792795 | |
| KIFC1 | 2.048040416 | 1.19E−06 | 0.000116485 | 1.139757476 | 2.437878232 | |
| PTMS | 1.556394667 | 1.24E−06 | 0.000120768 | 19.1542684 | 50.72354737 | Yes |
| UBASH3B | 1.896874413 | 1.37E−06 | 0.000131202 | 3.828418912 | 14.88145069 | |
| NUSAP1 | 1.471883402 | 1.54E−06 | 0.000146458 | 50.15568704 | 89.48275263 | |
| CD300A | −1.793079022 | 1.74E−06 | 0.000162542 | 90.8459596 | 25.90228042 | Yes |
| TPX2 | 1.99097788 | 1.92E−06 | 0.000178166 | 4.851452292 | 24.50750737 | |
| AURKA | 1.660078116 | 2.03E−06 | 0.00018727 | 17.55234268 | 26.20345458 | |
| KIF5C | 1.774252318 | 2.20E−06 | 0.000197891 | 2.414951648 | 7.161932421 | Yes |
| VDR | 1.916130686 | 2.32E−06 | 0.000204746 | 3.135480952 | 10.44611358 | |
| SYNJ2 | 1.952272272 | 2.36E−06 | 0.000206522 | 1.49190332 | 4.358019632 | |
| ATP10A | 1.719330489 | 2.48E−06 | 0.000213579 | 1.708745156 | 6.061125495 | |
| ANKRD35 | 1.989093325 | 2.82E−06 | 0.00024034 | 2.90909116 | 12.88178263 | |
| KLRC3 | 1.732307354 | 3.39E−06 | 0.000282668 | 38.1205408 | 104.6819184 | |
| SCCPDH | 1.267108566 | 4.37E−06 | 0.000357373 | 42.22486096 | 81.38209368 | |
| KIAA0101 | 1.705284926 | 5.92E−06 | 0.000467234 | 34.92798628 | 57.27524105 | Yes |
| CHN1 | 1.976034237 | 5.99E−06 | 0.000467565 | 8.04594322 | 65.83489663 | Yes |
| GTSE1 | 1.845443965 | 6.08E−06 | 0.000471895 | 2.67869814 | 5.922737205 | |
| ICAM2 | −1.574882829 | 6.48E−06 | 0.000499591 | 51.860032 | 16.69807895 | Yes |
| TTC24 | 1.676924085 | 7.49E−06 | 0.000570348 | 15.26772828 | 48.94895789 | |
| ZC3H12C | 2.032896561 | 7.69E−06 | 0.000578224 | 0.060009268 | 1.357966053 | |
| DKK3 | −1.679421385 | 8.50E−06 | 0.000631421 | 85.16482245 | 33.83001479 | |
| SARDH | 1.907804454 | 1.06E−05 | 0.000768356 | 10.17270494 | 48.39059842 | Yes |
| ZBED2 | 1.958522644 | 1.07E−05 | 0.000775184 | 8.41407728 | 44.29435947 | |
| DPF3 | 1.994370435 | 1.10E−05 | 0.00078556 | 0.155604456 | 4.017322021 | |
| ARHGEF12 | 1.884195097 | 1.15E−05 | 0.000815589 | 1.720978192 | 6.359689242 | |
| CHEK1 | 1.633053468 | 1.15E−05 | 0.000815589 | 1.72233128 | 6.573517947 | |
| SLC2A8 | 1.898875877 | 1.19E−05 | 0.000828911 | 5.26762768 | 32.67995121 | |
| PLAGL1 | 1.977412408 | 1.25E−05 | 0.00086325 | 0.678131668 | 8.558824895 | |
| IL15 | −1.776336936 | 1.39E−05 | 0.000953245 | 8.955241728 | 1.929823932 | |
| CXCL16 | −1.465177681 | 1.42E−05 | 0.000968842 | 33.54755604 | 13.30089653 | |
| LILRP2 | 1.929179014 | 1.72E−05 | 0.00115957 | 0.24319804 | 8.555959895 | |
| UAP1L1 | 1.635659609 | 2.06E−05 | 0.001350032 | 4.842522948 | 10.79838142 | Yes |
| EOMES | −1.082992213 | 2.14E−05 | 0.001392357 | 216.4456344 | 112.0899995 | Yes |
| XCL2 | 1.022838013 | 2.27E−05 | 0.001461407 | 117.1022852 | 217.8741316 | |
| FANCI | 1.767956945 | 2.29E−05 | 0.001462592 | 5.771166696 | 18.30659889 | |
| CDT1 | 1.563740306 | 2.48E−05 | 0.00157686 | 2.84855772 | 6.826375684 | |
| CACNA2D2 | −1.806533258 | 2.50E−05 | 0.001578903 | 1.939408892 | 0.800522095 | |
| TNFSF4 | 1.864855811 | 2.57E−05 | 0.001607152 | 8.7365456 | 41.49277777 | |
| ABAT | 1.714711942 | 2.59E−05 | 0.001607152 | 1.998721984 | 5.892387211 | |
| AHI1 | 1.38245204 | 2.59E−05 | 0.001607152 | 12.14165304 | 27.49229789 | Yes |
| ASB2 | 1.517671152 | 2.61E−05 | 0.00161221 | 13.9961398 | 43.76039474 | |
| DBN1 | 1.423942324 | 2.66E−05 | 0.001630949 | 8.21897794 | 19.68978158 | |
| LGMN | −1.534420984 | 2.72E−05 | 0.001663415 | 51.97189676 | 13.32886032 | |
| PTGIS | 1.360710073 | 3.22E−05 | 0.001920191 | 1.0126632 | 4.015272263 | |
| DFNB31 | 1.751477613 | 3.52E−05 | 0.00204918 | 2.68782576 | 11.29693105 | Yes |
| ABCB1 | 1.289757669 | 3.51E−05 | 0.00204918 | 17.53565533 | 46.09189158 | |
| ATP10D | 1.408338045 | 3.94E−05 | 0.002261703 | 8.2785482 | 18.62304158 | Yes |
| FCRL3 | −1.634974976 | 3.94E−05 | 0.002261703 | 57.74915068 | 22.82451037 | Yes |
| SPINT2 | −1.670867169 | 4.03E−05 | 0.002304224 | 81.86661636 | 19.88341084 | |
| LYZ | −1.206783578 | 4.12E−05 | 0.0023407 | 533.8098288 | 197.5742774 | |
| GCNT1 | 1.590291886 | 4.33E−05 | 0.002452984 | 8.729126624 | 25.69184137 | |
| NUAK2 | −1.164676934 | 4.57E−05 | 0.002563473 | 15.3906328 | 5.075766368 | Yes |
| SPNS3 | 1.583706166 | 4.60E−05 | 0.002564261 | 9.59597132 | 25.19064737 | |
| PTGDR | −1.106484699 | 4.61E−05 | 0.002564261 | 47.7035032 | 22.43134053 | |
| SOCS3 | −1.176968482 | 4.94E−05 | 0.00271839 | 130.4092708 | 54.63775147 | |
| KLRAP1 | −1.668786132 | 4.93E−05 | 0.00271839 | 31.6027852 | 8.668786158 | Yes |
| RUNX2 | 1.004705254 | 5.13E−05 | 0.002789748 | 21.72283 | 46.77662105 | |
| SVIL | −1.730549769 | 5.18E−05 | 0.002803752 | 6.2219778 | 0.869931489 | Yes |
| KLRB1 | 1.243451233 | 5.22E−05 | 0.002809517 | 249.3302182 | 490.05 | |
| LINC00963 | 1.231289717 | 5.46E−05 | 0.002923658 | 15.691546 | 35.08958263 | |
| AMICA1 | 1.140277208 | 5.47E−05 | 0.002923658 | 158.6033022 | 366.6787895 | Yes |
| CAMK1 | 1.691816551 | 5.89E−05 | 0.00313345 | 12.76232984 | 54.144063 | |
| ANKS1B | 1.817118618 | 5.94E−05 | 0.003144996 | 0.373814604 | 2.183310789 | |
| TMEM200A | 1.542695194 | 6.20E−05 | 0.003235193 | 10.90489994 | 25.15744737 | Yes |
| PGLYRP2 | 1.516783203 | 6.17E−05 | 0.003235193 | 2.53230324 | 5.937835789 | Yes |
| TTN-AS1 | −1.265199771 | 6.26E−05 | 0.003235193 | 3.374815664 | 1.286795842 | |
| PRSS23 | −1.409768113 | 6.38E−05 | 0.003281671 | 9.47118448 | 1.732799947 | Yes |
| SUOX | 1.791011507 | 6.48E−05 | 0.003317961 | 2.148762428 | 16.12955742 | |
| KCNK5 | 1.782686156 | 6.88E−05 | 0.003491967 | 3.473615356 | 20.51417421 | |
| DIXDC1 | 1.621869122 | 7.38E−05 | 0.003717195 | 0.632839116 | 3.195448053 | |
| CD28 | −1.118957638 | 7.48E−05 | 0.003749953 | 77.15902102 | 30.25053684 | |
| KIF14 | 1.61077793 | 7.88E−05 | 0.003902254 | 0.70509062 | 3.265626621 | |
| SNAP47 | 1.087540861 | 8.59E−05 | 0.004188653 | 45.1029988 | 73.56212632 | |
| ABCA1 | −1.727318199 | 9.08E−05 | 0.004411705 | 4.982207032 | 0.730471247 | |
| ITGA5 | −1.378404403 | 9.24E−05 | 0.004468571 | 36.65399692 | 11.21532078 | Yes |
| SYNGR3 | 1.663443022 | 0.000110901 | 0.005290731 | 10.13336744 | 42.42283158 | |
| RBBP9 | 1.35864834 | 0.000116888 | 0.0054847 | 3.0743766 | 7.297156368 | |
| SATB1 | −1.222543971 | 0.000116923 | 0.0054847 | 21.52242938 | 10.02693033 | Yes |
| C1orf106 | 1.654127927 | 0.000117558 | 0.005493609 | 0.429621136 | 2.195005284 | |
| DENND4A | −1.058190967 | 0.000120038 | 0.005546478 | 22.5608188 | 12.16038737 | |
| FASLG | 1.059205283 | 0.000127409 | 0.00586509 | 149.7843665 | 263.5392211 | |
| RGS16 | 1.695730781 | 0.000132589 | 0.006058347 | 9.466770612 | 44.55431789 | |
| CCNA2 | 1.63474981 | 0.000137262 | 0.006225719 | 16.76836842 | 30.29312263 | |
| SLC16A6 | 1.705711712 | 0.000139293 | 0.006263427 | 1.215146864 | 7.487247474 | |
| BAZ2B | −1.004936545 | 0.000139515 | 0.006263427 | 22.017383 | 11.41999521 | |
| CCRL2 | 1.61789572 | 0.000142927 | 0.006388745 | 18.98001112 | 41.17150789 | |
| COL6A2 | −1.68505588 | 0.000143773 | 0.006403357 | 5.798380532 | 0.906108884 | |
| PXN | −1.349346038 | 0.000144295 | 0.006403473 | 60.2272196 | 19.01642169 | |
| GINS1 | 1.561048491 | 0.000151389 | 0.006694195 | 2.35536462 | 9.327755947 | |
| ACP5 | 1.360154914 | 0.000160861 | 0.007023499 | 68.29268804 | 166.9910632 | |
| BCL2L11 | 1.353352837 | 0.000161682 | 0.007023499 | 6.783699552 | 20.68451776 | |
| USP14 | 1.141451467 | 0.000161566 | 0.007023499 | 37.82807232 | 60.20166842 | |
| MIR155HG | 1.568103062 | 0.000171693 | 0.007354789 | 17.03474912 | 61.13262474 | Yes |
| TK1 | 1.556990809 | 0.000172831 | 0.007377908 | 34.81022376 | 62.27577368 | |
| BIRC5 | 1.52789537 | 0.000175728 | 0.007475706 | 17.88640092 | 21.82443158 | |
| CRIP2 | −1.682549856 | 0.000176976 | 0.007502923 | 10.84884325 | 1.230886568 | |
| TTYH3 | 1.550941892 | 0.000194697 | 0.00814229 | 1.855617216 | 6.575156084 | |
| CASC5 | 1.683852608 | 0.000202408 | 0.008379571 | 0.7152126 | 6.408608889 | |
| HELLS | 1.194430839 | 0.000203562 | 0.008399136 | 6.51769844 | 11.66888526 | |
| NHS | 1.677210619 | 0.000219388 | 0.008962238 | 0.385406112 | 3.465654453 | |
| CENPU | 1.383262737 | 0.000221289 | 0.008983776 | 8.1349832 | 21.60639474 | |
| AMY2B | −1.142210726 | 0.000221372 | 0.008983776 | 10.3340532 | 4.278311474 | |
| BRCA1 | 1.317781531 | 0.000223203 | 0.009028397 | 1.784135444 | 4.474544053 | |
| PDLIM7 | 1.491977557 | 0.00022575 | 0.009101574 | 10.8833996 | 31.69171263 | |
| FAM84B | −1.321001087 | 0.000243726 | 0.009762507 | 9.7799954 | 3.669650684 | |
| C15orf53 | 1.602472447 | 0.000248983 | 0.009940784 | 3.78734548 | 11.796093 | Yes |
| HHLA3 | −1.453124988 | 0.000260044 | 0.010282577 | 26.8391892 | 7.859869105 | Yes |
| INPP5F | 1.557089828 | 0.00026203 | 0.010295127 | 4.56110998 | 14.65299721 | Yes |
| APEX2 | 1.150826136 | 0.000317738 | 0.012326832 | 26.07538084 | 42.41617368 | |
| ADRB2 | −1.371983994 | 0.000317481 | 0.012326832 | 116.3136846 | 40.39521814 | Yes |
| THEM4 | −1.136585144 | 0.000331488 | 0.012779916 | 22.07544464 | 9.964039 | |
| MCM2 | 1.417554569 | 0.000357578 | 0.013573646 | 12.7239374 | 34.84616211 | |
| PDLIM1 | −1.20806686 | 0.000356537 | 0.013573646 | 60.56773384 | 26.39405689 | |
| HJURP | 1.512082809 | 0.000361013 | 0.013662027 | 3.0432999 | 10.34542453 | |
| RDH10 | 1.392983915 | 0.000374495 | 0.014000421 | 4.7784252 | 13.67609332 | |
| FUT8 | 1.270544325 | 0.000388012 | 0.014375073 | 16.45712298 | 39.12060211 | |
| MKI67 | 1.482800913 | 0.000403173 | 0.01475575 | 3.740788684 | 9.95709 | Yes |
| CDHR3 | −1.220422843 | 0.000409235 | 0.014893018 | 4.32965516 | 1.594811011 | |
| RYBP | 1.530157948 | 0.000426073 | 0.015369766 | 1.76679696 | 6.359807421 | |
| MYO1E | 1.582425887 | 0.000450856 | 0.016029436 | 3.620583316 | 15.26436368 | |
| TOP2A | 1.352846874 | 0.000459462 | 0.016288455 | 14.67442033 | 32.63963384 | |
| NDFIP2 | 1.116525313 | 0.000467494 | 0.016478506 | 40.7288212 | 91.40315263 | |
| MAD2L2 | 1.121501594 | 0.00048711 | 0.017121003 | 80.19018184 | 176.2801105 | |
| BRCA2 | 1.417780448 | 0.00049781 | 0.017436904 | 1.666127432 | 4.232477479 | |
| WDTC1 | −1.383667482 | 0.000498958 | 0.017436904 | 7.652969292 | 3.021814247 | |
| ITGA1 | 1.132457545 | 0.000525477 | 0.018037457 | 53.19740808 | 109.6885368 | Yes |
| PLEKHG3 | −1.568835408 | 0.0005408 | 0.018481593 | 7.94662748 | 0.826099621 | Yes |
| MAP3K6 | 1.405480363 | 0.000557245 | 0.018886615 | 2.83304806 | 7.054325263 | |
| AASS | −1.263208166 | 0.000575905 | 0.019306886 | 11.31210144 | 2.225013795 | |
| IL18BP | −1.194180888 | 0.000582721 | 0.019482467 | 54.67915012 | 21.38025753 | |
| RGS12 | −1.099836473 | 0.000595214 | 0.019792869 | 3.29526236 | 0.982810789 | |
| CD14 | −1.424675215 | 0.000606273 | 0.02010642 | 67.70359144 | 14.33756879 | Yes |
| KRT81 | 1.555202248 | 0.000612805 | 0.020127568 | 2.24269256 | 18.08521053 | |
| GALNT2 | 1.207810735 | 0.000613436 | 0.020127568 | 21.1598464 | 43.74474211 | |
| MPST | 1.049224887 | 0.000613323 | 0.020127568 | 22.35174136 | 37.2394 | |
| PATL2 | −1.117057136 | 0.000612364 | 0.020127568 | 85.39035448 | 37.18099205 | Yes |
| CEP41 | 1.008732155 | 0.000633389 | 0.020727119 | 3.8788086 | 8.499325737 | |
| ELK1 | 1.299109563 | 0.000643899 | 0.021015287 | 10.29477778 | 20.22793895 | |
| PPP1R21 | 1.01536727 | 0.000714978 | 0.022910882 | 21.55712479 | 36.39565316 | |
| DHFR | 1.364150744 | 0.000717621 | 0.022935974 | 6.949739276 | 16.97895126 | |
| CCDC50 | 1.32032169 | 0.000721961 | 0.023015076 | 4.642402768 | 8.387248079 | |
| PDE7B | 1.258502263 | 0.0007291 | 0.023019759 | 1.530637224 | 4.094892895 | |
| BMPR1B | −1.192565663 | 0.000729572 | 0.023019759 | 2.2447436 | 0.246532374 | |
| CCL18 | −1.43447936 | 0.00074364 | 0.023344239 | 120.7602916 | 32.81444384 | |
| KIAA1524 | 1.421479171 | 0.000758263 | 0.023742862 | 4.381154288 | 13.40530519 | |
| ATL2 | 1.147738802 | 0.00079133 | 0.024345745 | 7.3282878 | 13.66836579 | Yes |
| CARD6 | 1.29906251 | 0.000813151 | 0.024892909 | 1.37840932 | 6.053855789 | |
| ZNF514 | −1.43484038 | 0.000832933 | 0.025372588 | 5.027664092 | 0.490173568 | |
| AGPS | 1.217710921 | 0.000852721 | 0.025776769 | 7.239435836 | 16.10707737 | |
| C1QC | −1.289800192 | 0.000850368 | 0.025776769 | 122.636079 | 59.72089795 | |
| CDKN2C | 1.303116031 | 0.000901495 | 0.026984604 | 11.7002326 | 27.63487263 | |
| CD200R1 | 1.029994841 | 0.000903351 | 0.026984604 | 45.1439298 | 88.27228947 | |
| SLC4A2 | 1.214325475 | 0.000956432 | 0.027894817 | 7.793165208 | 15.40505526 | |
| ACSL4 | 1.044437072 | 0.000960786 | 0.027955697 | 19.3627672 | 34.15788474 | Yes |
| LOC101928988 | 1.400426941 | 0.000984987 | 0.028458522 | 5.24736404 | 13.66175105 | |
| CDC6 | 1.282112236 | 0.000983324 | 0.028458522 | 4.7555254 | 6.325555684 | |
| LOC100996286 | −1.376533152 | 0.001018637 | 0.029157611 | 60.23214176 | 18.27346626 | Yes |
| C1orf162 | −1.321986663 | 0.001023341 | 0.029224437 | 149.498455 | 55.57553158 | |
| LRRN3 | 1.465688528 | 0.001065437 | 0.030286388 | 2.271649976 | 29.283488 | |
| TET2 | 1.119528008 | 0.001084458 | 0.030756213 | 6.91857828 | 11.33880421 | |
| ZWINT | 1.400223352 | 0.001092088 | 0.030901593 | 19.685767 | 40.97230947 | |
| TNFRSF18 | 1.436215773 | 0.001123689 | 0.031578469 | 20.86098484 | 73.40014211 | |
| APOE | −1.254820747 | 0.001173695 | 0.032612345 | 125.2921753 | 55.44808074 | |
| RHOC | 1.010556359 | 0.001187599 | 0.032850683 | 64.81549196 | 137.4523789 | |
| PTP4A3 | −1.470184527 | 0.001202261 | 0.033181871 | 10.16620279 | 1.326568737 | |
| PALB2 | 1.441779748 | 0.001209152 | 0.033258812 | 3.711650348 | 11.35856868 | |
| RAP1GAP2 | −1.46757526 | 0.001210441 | 0.033258812 | 3.335396492 | 0.336865295 | Yes |
| PMM2 | 1.057768261 | 0.001233653 | 0.033789307 | 20.37359668 | 30.27954895 | |
| EPSTI1 | 1.148188972 | 0.001238521 | 0.033804499 | 20.8874532 | 45.41434368 | |
| WIPF3 | 1.394904839 | 0.001249282 | 0.033969958 | 4.911925736 | 18.05181664 | |
| PLAUR | −1.41499774 | 0.00125009 | 0.033969958 | 88.264021 | 20.07343137 | Yes |
| LINC00539 | 1.398270111 | 0.00126082 | 0.034111277 | 12.49131944 | 34.69323995 | |
| LIMK1 | 1.300030117 | 0.00127323 | 0.034296578 | 9.1342948 | 23.865563 | |
| MAN1A1 | 1.085325998 | 0.00130679 | 0.034847713 | 10.56250701 | 21.17127137 | |
| SAC3D1 | 1.302130097 | 0.0013855 | 0.036523325 | 5.48930056 | 16.32300211 | |
| CKAP2L | 1.321005047 | 0.001420909 | 0.036776193 | 3.42151756 | 7.193064868 | |
| NAIF1 | 1.139889907 | 0.001417708 | 0.036776193 | 1.77297092 | 4.622296579 | |
| AMZ1 | 1.401084929 | 0.001430853 | 0.036835065 | 0.757580336 | 4.697318947 | |
| ZDHHC18 | 1.322634784 | 0.001433155 | 0.036835065 | 3.679985244 | 8.532325263 | |
| CST3 | −1.206589704 | 0.001485587 | 0.038024247 | 54.298094 | 30.77291768 | Yes |
| SDHAP1 | 1.169568814 | 0.001490256 | 0.038064774 | 4.97298268 | 9.654161053 | |
| SSH1 | 1.136181425 | 0.001535372 | 0.03886269 | 4.366832536 | 8.201638421 | |
| GINS3 | 1.431414228 | 0.001595634 | 0.039929684 | 0.8409542 | 6.411405879 | |
| NAV1 | −1.182098287 | 0.001601388 | 0.039940807 | 1.522865816 | 0.5266027 | |
| CASP9 | 1.19556286 | 0.001614154 | 0.040148821 | 9.795832856 | 11.05799316 | |
| SGK1 | −1.003855752 | 0.001621156 | 0.040241856 | 59.85603994 | 29.82423195 | |
| ITGA2 | 1.216143898 | 0.001632734 | 0.04036681 | 3.19618754 | 5.027070905 | |
| MZB1 | 1.309894553 | 0.001693951 | 0.041547254 | 24.83474744 | 56.75498526 | |
| KLF2 | −1.23514898 | 0.001707128 | 0.041626658 | 56.41764416 | 25.34566311 | |
| VCAM1 | 1.142380045 | 0.001725703 | 0.041909455 | 44.44741996 | 77.7627 | |
| TIAM2 | 1.421865316 | 0.001741213 | 0.042203047 | 0.52895268 | 3.874846842 | |
| SLC27A2 | 1.267279008 | 0.001756777 | 0.042334312 | 31.71734129 | 74.47079316 | Yes |
| ST8SIA1 | −1.004763422 | 0.001756924 | 0.042334312 | 6.43101508 | 3.163632211 | |
| PIAS2 | −1.272095252 | 0.001754315 | 0.042334312 | 10.40526076 | 3.268512858 | |
| XYLT1 | 1.365509161 | 0.001779998 | 0.042806697 | 1.1606454 | 2.969577158 | Yes |
| ADAMTS17 | 1.146044925 | 0.001798084 | 0.043073725 | 0.970106168 | 2.848200895 | |
| PLEKHA5 | −1.028909852 | 0.001810506 | 0.043287231 | 8.6564934 | 3.563287947 | |
| IL18RAP | 1.246342812 | 0.001856197 | 0.043892409 | 30.24285953 | 66.06705263 | |
| ACOT7 | 1.351789221 | 0.001882395 | 0.044403647 | 13.74920072 | 39.01015632 | |
| TYROBP | −1.130633801 | 0.001971061 | 0.046142286 | 286.6825624 | 111.0874333 | |
| SNX9 | 1.194321551 | 0.001980146 | 0.046267155 | 18.41411002 | 40.61670526 | |
| GPA33 | 1.400729896 | 0.001984058 | 0.046270937 | 4.352248768 | 17.61862789 | |
| UHRF1 | 1.171771134 | 0.002007933 | 0.046651354 | 4.08198472 | 9.493753579 | |
| GMNN | 1.30781058 | 0.002014754 | 0.046721835 | 40.01412236 | 62.66041737 | |
| CDCA3 | 1.390821103 | 0.002025511 | 0.046883172 | 2.010761628 | 9.040348842 | |
| RMND1 | 1.120686463 | 0.002045247 | 0.047251337 | 14.60276136 | 22.08568263 | |
| PDE4A | 1.129148503 | 0.002055599 | 0.047401735 | 3.8111694 | 7.638657368 | Yes |
| TRIM16 | −1.221863915 | 0.00210131 | 0.048185618 | 2.71523592 | 0.879359011 | |
| RIC1 | 1.123345605 | 0.002115833 | 0.048428633 | 4.893008408 | 9.940590942 | |
| CDKN3 | 1.30287349 | 0.002126804 | 0.048499774 | 33.00784028 | 71.41053842 | |
| KCTD9 | 1.333185286 | 0.002141187 | 0.048558511 | 6.9347676 | 29.03811263 | |
| EMC9 | 1.213045927 | 0.00213996 | 0.048558511 | 29.22870432 | 55.71777368 | |
| NUDT14 | 1.068661519 | 0.00215483 | 0.048778243 | 46.7673308 | 83.1857 | |
| SLC18A2 | −1.346962778 | 0.002163723 | 0.048800448 | 16.25625348 | 1.404258258 | |
| CD226 | 1.147445623 | 0.00217143 | 0.048810471 | 23.20268566 | 55.71804068 | Yes |
| AZIN2 | 1.373051169 | 0.002208751 | 0.049097951 | 3.109285076 | 13.45753084 | |
| CDK1 | 1.312177938 | 0.002227322 | 0.049332993 | 15.2390608 | 34.75512211 | |

TABLE 5
List of genes uniquely expressed in Tumor TRM

| Gene ID | | | |
|---|---|---|---|
| | log2FoldChange-vs- | log2FoldChange-vs- | log2FoldChange-vs- |
| | Lung-Non-TRM | Lung-TRM | Tumor-Non-TRM |
| GSG2 | 5.217896122 | 1.727403278 | 3.635833487 |
| MYO7A | 11.04811002 | 2.259998486 | 3.513567966 |
| LAYN | 7.695794138 | 4.310357629 | 3.621535741 |
| KRT86 | 6.065996193 | 2.036854575 | 3.381632722 |
| STMN1 | 1.440509864 | 1.315548427 | 1.366049689 |
| ENTPD1 | 3.816873481 | 3.015149779 | 2.51222904 |
| KLRC2 | 2.854407583 | 1.140028446 | 2.347036307 |
| TOX | 1.63476055 | 1.232637535 | 1.04690617 |
| TNS3 | 3.760885198 | 3.497445411 | 2.871610018 |
| SRGAP3 | 6.94255386 | 2.092844412 | 2.957823113 |
| CLNK | 8.145947295 | 1.421442605 | 2.981297463 |
| AFAP1L2 | 6.701024419 | 3.220993336 | 2.730768148 |
| AKAP5 | 3.365061373 | 2.50622053 | 2.119777167 |
| CXCL13 | 5.905835106 | 6.008906489 | 2.786215532 |
| HAVCR2 | 2.830747425 | 2.50361094 | 2.115630958 |
| KIR2DL4 | 5.015048432 | 2.424076288 | 2.739112566 |
| CHRM3-AS2 | 2.363451642 | 2.414451417 | 2.773184838 |
| TNFRSF9 | 2.408977601 | 3.457216272 | 2.018972638 |
| RRM2 | 3.354352762 | 2.459896628 | 2.032567257 |
| CD109 | 2.569939564 | 1.712328303 | 2.209733753 |
| DBH-AS1 | 3.363084813 | 2.112220802 | 2.446850699 |
| SIRPG | 1.906534643 | 2.9228103 | 1.173047291 |
| CCL3 | 1.367188475 | 1.746678602 | 1.308047955 |
| KIF2C | 4.589213359 | 3.985938676 | 2.340084386 |
| CTLA4 | 3.667516869 | 2.138134252 | 2.049857964 |
| IFITM10 | 3.604262638 | 2.307516511 | 2.259689528 |
| GEM | 3.079089548 | 4.530445048 | 2.209029285 |
| RAD51AP1 | 2.090366569 | 3.053062158 | 2.184111701 |
| KIFC1 | 2.481368317 | 2.80215291 | 2.048040416 |
| NUSAP1 | 2.851989004 | 1.698825624 | 1.471883402 |
| AURKA | 3.109026783 | 2.744353884 | 1.660078116 |
| VDR | 1.95356489 | 2.683453148 | 1.916130686 |
| KIAA0101 | 3.295801712 | 1.951171749 | 1.705284926 |
| ZC3H12C | 3.7603296 | 3.563030404 | 2.032896561 |
| ZBED2 | 5.239224614 | 4.760873221 | 1.958522644 |
| LILRP2 | 4.430404492 | 3.045769331 | 1.929179014 |
| FANCI | 1.852185073 | 2.161761079 | 1.767956945 |
| TNFSF4 | 3.517317663 | 2.849440698 | 1.864855811 |
| ASB2 | 3.153886441 | 2.744017787 | 1.517671152 |
| CAMK1 | 2.833585603 | 2.144045067 | 1.691816551 |
| ANKS1B | 2.773441919 | 3.802723742 | 1.817118618 |
| SUOX | 2.133564156 | 2.455137719 | 1.791011507 |
| KCNK5 | 4.46330601 | 2.53092669 | 1.782686156 |
| KIF14 | 2.380994181 | 2.894778174 | 1.61077793 |
| SYNGR3 | 7.183233568 | 2.03673754 | 1.663443022 |
| C1orf106 | 1.817676184 | 2.317507122 | 1.654127927 |
| CCNA2 | 2.582220087 | 4.786563234 | 1.63474981 |
| CCRL2 | 1.868021117 | 1.993576214 | 1.61789572 |
| GINS1 | 2.672407184 | 2.308923834 | 1.561048491 |
| TK1 | 2.003932836 | 2.116056331 | 1.556990809 |
| BIRC5 | 2.035908601 | 2.086973635 | 1.52789537 |
| CASC5 | 4.535303428 | 2.610124315 | 1.683852608 |
| INPP5F | 4.725026767 | 2.536080077 | 1.557089828 |
| MCM2 | 2.195434017 | 1.931278407 | 1.417554569 |
| HJURP | 4.247955681 | 2.482548456 | 1.512082809 |
| RDH10 | 1.465733095 | 1.575939474 | 1.392983915 |
| FUT8 | 2.205316587 | 1.36327465 | 1.270544325 |
| MKI67 | 5.540218705 | 2.619692627 | 1.482800913 |
| MYO1E | 5.860674228 | 3.123013282 | 1.582425887 |
| TOP2A | 2.592103178 | 2.29539434 | 1.352846874 |
| NDFIP2 | 2.713458817 | 1.680758608 | 1.116525313 |
| PDE7B | 2.536531597 | 2.200650041 | 1.258502263 |
| CDC6 | 2.141006594 | 1.532873841 | 1.282112236 |
| LOC1019289 | 3.758323048 | 1.797657182 | 1.400426941 |
| TNFRSF18 | 1.978937742 | 1.965403844 | 1.436215773 |
| WIPF3 | 4.099033144 | 3.849918125 | 1.394904839 |
| CKAP2L | 2.704714791 | 1.999536687 | 1.321005047 |
| AMZ1 | 4.135576107 | 2.360849448 | 1.401084929 |
| ITGA2 | 3.554125945 | 2.137378038 | 1.216143898 |
| MZB1 | 3.47569117 | 2.399099383 | 1.309894553 |
| VCAM1 | 3.276880266 | 2.615175589 | 1.142380045 |
| TIAM2 | 2.708362955 | 2.731877282 | 1.421865316 |
| SLC27A2 | 3.492322458 | 1.81432632 | 1.267279008 |
| UHRF1 | 2.369255423 | 2.106619252 | 1.171771134 |
| RIC1 | 1.504751576 | 2.018930511 | 1.123345605 |
| EMC9 | 1.162931851 | 2.837975396 | 1.213045927 |
| CDK1 | 2.597647929 | 1.787691839 | 1.312177938 |
| PLAC8 | −3.317514759 | −1.464685708 | −2.474542579 |
| FCGR3A | −5.118125315 | −3.03735392 | −2.930611162 |
| KLRF1 | −7.586330442 | −2.531583712 | −2.393123248 |
| DHRS3 | −1.984899007 | −1.576886916 | −1.538676923 |
| FCGR3B | −4.595506208 | −3.242872284 | −2.089580696 |
| CXCL16 | −3.57727385 | −2.899486306 | −1.465177681 |
| LYZ | −3.456823139 | −2.949409285 | −1.206783578 |
| SVIL | −3.938867287 | −2.316019064 | −1.730549769 |
| PXN | −2.85790707 | −2.148037695 | −1.349346038 |
| CRIP2 | −3.769688421 | −4.197779904 | −1.682549856 |
| CCL18 | −3.20866263 | −2.603917856 | −1.43447936 |
| C1QC | −3.265559876 | −2.572827616 | −1.289800192 |
| C1orf162 | −2.648782815 | −2.037386914 | −1.321986663 |
| PLAUR | −4.779211289 | −2.670399998 | −1.41499774 |
| TYROBP | −2.281337445 | −2.008092934 | −1.130633801 |
| | pvalue-vs- | pvalue-vs- | pvalue-vs- |
| | Lung-Non-TRM | Lung-TRM | Tumor-Non-TRM |
| GSG2 | 5.02E−26 | 0.000758938 | 2.36E−28 |
| MYO7A | 3.47E−107 | 1.34E−05 | 5.58E−18 |
| LAYN | 8.56E−39 | 5.03E−13 | 1.31E−16 |
| KRT86 | 7.30E−21 | 0.000900314 | 4.46E−16 |
| STMN1 | 2.16E−08 | 0.000695078 | 7.63E−16 |
| ENTPD1 | 2.22E−27 | 1.36E−18 | 1.22E−14 |
| KLRC2 | 5.53E−10 | 0.002517694 | 7.93E−14 |
| TOX | 2.55E−10 | 1.09E−06 | 2.72E−12 |
| TNS3 | 5.89E−09 | 2.77E−09 | 1.26E−11 |
| SRGAP3 | 5.11E−32 | 0.000112977 | 1.36E−11 |
| CLNK | 7.74E−35 | 0.001945149 | 1.56E−11 |
| AFAP1L2 | 5.37E−33 | 4.88E−08 | 2.03E−11 |
| AKAP5 | 2.16E−22 | 1.17E−13 | 7.69E−11 |
| CXCL13 | 1.94E−16 | 6.94E−23 | 7.81E−11 |
| HAVCR2 | 2.47E−13 | 2.37E−10 | 8.47E−11 |
| KIR2DL4 | 1.17E−17 | 9.34E−05 | 1.92E−10 |
| CHRM3-AS2 | 0.000321447 | 3.49E−06 | 4.89E−10 |
| TNFRSF9 | 4.92E−10 | 1.38E−17 | 6.12E−10 |
| RRM2 | 2.59E−12 | 2.37E−08 | 4.49E−09 |
| CD109 | 5.35E−09 | 0.000244747 | 8.57E−09 |
| DBH-AS1 | 9.85E−05 | 0.002055901 | 1.11E−08 |
| SIRPG | 2.69E−07 | 6.57E−13 | 1.38E−08 |
| CCL3 | 1.14E−05 | 1.34E−07 | 2.27E−08 |
| KIF2C | 1.15E−10 | 2.19E−10 | 3.24E−08 |
| CTLA4 | 7.06E−17 | 1.34E−07 | 3.17E−07 |
| IFITM10 | 2.56E−08 | 8.36E−05 | 5.26E−07 |
| GEM | 0.000154922 | 3.22E−10 | 1.15E−06 |
| RAD51AP1 | 0.001952484 | 3.23E−05 | 1.17E−06 |
| KIFC1 | 9.82E−05 | 6.16E−06 | 1.19E−06 |
| NUSAP1 | 3.66E−07 | 5.28E−08 | 1.54E−06 |
| AURKA | 5.48E−14 | 1.75E−09 | 2.03E−06 |
| VDR | 0.000531623 | 5.48E−07 | 2.32E−06 |
| KIAA0101 | 1.04E−16 | 2.33E−05 | 5.92E−06 |
| ZC3H12C | 6.05E−06 | 1.52E−06 | 7.69E−06 |
| ZBED2 | 6.59E−13 | 1.64E−13 | 1.07E−05 |
| LILRP2 | 4.75E−11 | 1.91E−06 | 1.72E−05 |
| FANCI | 1.65E−05 | 3.98E−05 | 2.29E−05 |
| TNFSF4 | 6.96E−07 | 2.30E−07 | 2.57E−05 |
| ASB2 | 1.09E−06 | 1.21E−08 | 2.61E−05 |
| CAMK1 | 7.88E−07 | 0.000110133 | 5.89E−05 |
| ANKS1B | 0.00126077 | 1.57E−06 | 5.94E−05 |
| SUOX | 0.001029801 | 0.000762109 | 6.48E−05 |
| KCNK5 | 4.16E−11 | 0.000251743 | 6.88E−05 |
| KIF14 | 7.48E−07 | 2.19E−07 | 7.88E−05 |
| SYNGR3 | 5.69E−33 | 0.001004625 | 0.000110901 |
| C1orf106 | 0.001986359 | 0.000131969 | 0.000117558 |
| CCNA2 | 7.12E−05 | 1.25E−13 | 0.000137262 |
| CCRL2 | 0.005883769 | 0.002902786 | 0.000142927 |
| GINS1 | 2.42E−10 | 1.62E−06 | 0.000151389 |
| TK1 | 0.001539824 | 0.000225416 | 0.000172831 |
| BIRC5 | 0.000671717 | 0.000761326 | 0.000175728 |
| CASC5 | 7.91E−11 | 0.000117916 | 0.000202408 |
| INPP5F | 3.54E−14 | 7.06E−06 | 0.00026203 |
| MCM2 | 8.88E−05 | 0.000448689 | 0.000357578 |
| HJURP | 2.82E−23 | 9.34E−07 | 0.000361013 |
| RDH10 | 0.00736061 | 0.001570757 | 0.000374495 |
| FUT8 | 4.52E−06 | 0.001561597 | 0.000388012 |
| MKI67 | 5.85E−17 | 3.30E−05 | 0.000403173 |
| MYO1E | 1.73E−21 | 1.47E−06 | 0.000450856 |
| TOP2A | 9.06E−06 | 4.98E−05 | 0.000459462 |
| NDFIP2 | 1.26E−08 | 0.000537766 | 0.000467494 |
| PDE7B | 1.36E−10 | 9.45E−06 | 0.0007291 |
| CDC6 | 1.27E−06 | 0.000175457 | 0.000983324 |
| LOC1019289 | 1.57E−08 | 0.001917135 | 0.000984987 |
| TNFRSF18 | 0.001847341 | 0.000382211 | 0.001123689 |
| WIPF3 | 2.68E−14 | 4.03E−14 | 0.001249282 |
| CKAP2L | 7.33E−06 | 0.000608346 | 0.001420909 |
| AMZ1 | 2.88E−10 | 0.000875684 | 0.001430853 |
| ITGA2 | 2.23E−16 | 0.000112489 | 0.001632734 |
| MZB1 | 9.58E−10 | 3.77E−05 | 0.001693951 |
| VCAM1 | 2.92E−08 | 2.67E−05 | 0.001725703 |
| TIAM2 | 0.00031317 | 0.000187503 | 0.001741213 |
| SLC27A2 | 1.53E−07 | 0.001644883 | 0.001756777 |
| UHRF1 | 5.16E−09 | 3.57E−07 | 0.002007933 |
| RIC1 | 0.008526297 | 0.001650318 | 0.002115833 |
| EMC9 | 0.001576823 | 3.51E−07 | 0.00213996 |
| CDK1 | 0.00030535 | 0.002941176 | 0.002227322 |
| PLAC8 | 7.58E−15 | 0.002185141 | 7.61E−17 |
| FCGR3A | 1.41E−19 | 1.95E−07 | 1.81E−13 |
| KLRF1 | 2.28E−44 | 0.001366298 | 1.07E−07 |
| DHRS3 | 5.09E−12 | 0.000251292 | 2.94E−07 |
| FCGR3B | 1.28E−16 | 4.90E−08 | 4.25E−07 |
| CXCL16 | 2.76E−21 | 2.41E−10 | 1.42E−05 |
| LYZ | 5.86E−25 | 3.71E−19 | 4.12E−05 |
| SVIL | 1.70E−17 | 1.93E−05 | 5.18E−05 |
| PXN | 5.41E−13 | 9.50E−07 | 0.000144295 |
| CRIP2 | 6.48E−10 | 1.44E−15 | 0.000176976 |
| CCL18 | 1.17E−07 | 0.000146058 | 0.00074364 |
| C1QC | 3.39E−13 | 1.62E−06 | 0.000850368 |
| C1orf162 | 1.11E−09 | 3.06E−06 | 0.001023341 |
| PLAUR | 4.56E−19 | 2.63E−05 | 0.00125009 |
| TYROBP | 1.04E−06 | 1.71E−05 | 0.001971061 |
| | padj-vs- | padj-vs- | padj-vs- |
| | Lung-Non-TRM | Lung-TRM | Tumor-Non-TRM |
| GSG2 | 3.35E−23 | 0.017682536 | 7.27E−25 |
| MYO7A | 4.39E−103 | 0.000734598 | 8.61E−15 |
| LAYN | 1.36E−35 | 2.07E−10 | 1.47E−13 |
| KRT86 | 2.25E−18 | 0.020077613 | 4.58E−13 |
| STMN1 | 8.43E−07 | 0.016592582 | 6.72E−13 |
| ENTPD1 | 1.65E−24 | 1.99E−15 | 7.94E−12 |
| KLRC2 | 3.14E−08 | 0.040436251 | 4.08E−11 |
| TOX | 1.51E−08 | 9.50E−05 | 1.20E−09 |
| TNS3 | 2.72E−07 | 5.22E−07 | 5.17E−09 |
| SRGAP3 | 5.39E−29 | 0.004201487 | 5.42E−09 |
| CLNK | 1.09E−31 | 0.034357992 | 5.82E−09 |
| AFAP1L2 | 6.55E−30 | 6.81E−06 | 7.35E−09 |
| AKAP5 | 9.43E−20 | 5.93E−11 | 2.15E−08 |
| CXCL13 | 3.19E−14 | 4.58E−19 | 2.15E−08 |
| HAVCR2 | 2.58E−11 | 5.90E−08 | 2.22E−08 |
| KIR2DL4 | 2.47E−15 | 0.003617679 | 4.82E−08 |
| CHRM3-AS2 | 0.003597258 | 0.000248846 | 1.16E−07 |
| TNFRSF9 | 2.83E−08 | 1.83E−14 | 1.35E−07 |
| RRM2 | 2.22E−10 | 3.64E−06 | 8.03E−07 |
| CD109 | 2.51E−07 | 0.00737706 | 1.47E−06 |
| DBH-AS1 | 0.001337201 | 0.035666244 | 1.84E−06 |
| SIRPG | 8.23E−06 | 2.63E−10 | 2.24E−06 |
| CCL3 | 0.000214864 | 1.59E−05 | 3.37E−06 |
| KIF2C | 7.30E−09 | 5.68E−08 | 4.65E−06 |
| CTLA4 | 1.28E−14 | 1.59E−05 | 3.76E−05 |
| IFITM10 | 9.82E−07 | 0.003365354 | 5.64E−05 |
| GEM | 0.001972409 | 7.73E−08 | 0.000114883 |
| RAD51AP1 | 0.015324702 | 0.00153112 | 0.00011594 |
| KIFC1 | 0.001334843 | 0.000383892 | 0.000116485 |
| NUSAP1 | 1.09E−05 | 7.26E−06 | 0.000146458 |
| AURKA | 6.25E−12 | 3.45E−07 | 0.00018727 |
| VDR | 0.005413661 | 5.18E−05 | 0.000204746 |
| KIAA0101 | 1.85E−14 | 0.001161096 | 0.000467234 |
| ZC3H12C | 0.000126191 | 0.000125603 | 0.000578224 |
| ZBED2 | 6.28E−11 | 7.45E−11 | 0.000775184 |
| LILRP2 | 3.27E−09 | 0.0001461 | 0.00115957 |
| FANCI | 0.000295897 | 0.001812139 | 0.001462592 |
| TNFSF4 | 1.95E−05 | 2.48E−05 | 0.001607152 |
| ASB2 | 2.88E−05 | 1.90E−06 | 0.00161221 |
| CAMK1 | 2.16E−05 | 0.004142382 | 0.00313345 |
| ANKS1B | 0.010841195 | 0.000127578 | 0.003144996 |
| SUOX | 0.00923904 | 0.017682536 | 0.003317961 |
| KCNK5 | 2.93E−09 | 0.007520596 | 0.003491967 |
| KIF14 | 2.07E−05 | 2.41E−05 | 0.003902254 |
| SYNGR3 | 6.55E−30 | 0.021651416 | 0.005290731 |
| C1orf106 | 0.015542433 | 0.004671714 | 0.005493609 |
| CCNA2 | 0.001016184 | 6.01E−11 | 0.006225719 |
| CCRL2 | 0.035886174 | 0.044354845 | 0.006388745 |
| GINS1 | 1.44E−08 | 0.000129199 | 0.006694195 |
| TK1 | 0.012782761 | 0.00696941 | 0.007377908 |
| BIRC5 | 0.006540595 | 0.017682536 | 0.007475706 |
| CASC5 | 5.14E−09 | 0.004265019 | 0.008379571 |
| INPP5F | 4.15E−12 | 0.000431737 | 0.010295127 |
| MCM2 | 0.001228037 | 0.011894768 | 0.013573646 |
| HJURP | 1.32E−20 | 8.50E−05 | 0.013662027 |
| RDH10 | 0.042395964 | 0.029400015 | 0.014000421 |
| FUT8 | 9.81E−05 | 0.029400015 | 0.014375073 |
| MKI67 | 1.07E−14 | 0.001551665 | 0.01475575 |
| MYO1E | 6.64E−19 | 0.000121737 | 0.016029436 |
| TOP2A | 0.000178288 | 0.002190691 | 0.016288455 |
| NDFIP2 | 5.27E−07 | 0.013620929 | 0.016478506 |
| PDE7B | 8.43E−09 | 0.000551192 | 0.023019759 |
| CDC6 | 3.27E−05 | 0.005879161 | 0.028458522 |
| LOC1019289 | 6.32E−07 | 0.033973184 | 0.028458522 |
| TNFRSF18 | 0.014728033 | 0.01044711 | 0.031578469 |
| WIPF3 | 3.17E−12 | 2.29E−11 | 0.033969958 |
| CKAP2L | 0.000147773 | 0.015040042 | 0.036776193 |
| AMZ1 | 1.69E−08 | 0.019661184 | 0.036835065 |
| ITGA2 | 3.62E−14 | 0.004195126 | 0.04036681 |
| MZB1 | 5.14E−08 | 0.001740835 | 0.041547254 |
| VCAM1 | 1.11E−06 | 0.001304561 | 0.041909455 |
| TIAM2 | 0.003517055 | 0.006107399 | 0.042203047 |
| SLC27A2 | 4.94E−06 | 0.030514706 | 0.042334312 |
| UHRF1 | 2.43E−07 | 3.57E−05 | 0.046651354 |
| RIC1 | 0.04745656 | 0.030514706 | 0.048428633 |
| EMC9 | 0.01302164 | 3.57E−05 | 0.048558511 |
| CDK1 | 0.003453732 | 0.044785941 | 0.049332993 |
| PLAC8 | 9.79E−13 | 0.037207354 | 9.38E−14 |
| FCGR3A | 3.65E−17 | 2.20E−05 | 8.59E−11 |
| KLRF1 | 4.80E−41 | 0.026882065 | 1.41E−05 |
| DHRS3 | 4.10E−10 | 0.007520596 | 3.52E−05 |
| FCGR3B | 2.23E−14 | 6.81E−06 | 4.77E−05 |
| CXCL16 | 9.77E−19 | 5.90E−08 | 0.000968842 |
| LYZ | 3.71E−22 | 6.99E−16 | 0.0023407 |
| SVIL | 3.54E−15 | 0.001007621 | 0.002803752 |
| PXN | 5.35E−11 | 8.53E−05 | 0.006403473 |
| CRIP2 | 3.63E−08 | 1.18E−12 | 0.007502923 |
| CCL18 | 3.93E−06 | 0.005101201 | 0.023344239 |
| C1QC | 3.49E−11 | 0.000129199 | 0.025776769 |
| C1orf162 | 5.83E−08 | 0.000223456 | 0.029224437 |
| PLAUR | 1.13E−16 | 0.001290023 | 0.033969958 |
| TYROBP | 2.77E−05 | 0.000915616 | 0.046142286 |
| | MeanTPM- | MeanTPM_ | MeanTPM_ |
| | Tumor-TRM | NIL_CD103neg | NIL_CD103pos |
| GSG2 | 10.79137868 | 0.161439895 | 8.00016295 |
| MYO7A | 44.23771526 | 0.038636476 | 13.82020979 |
| LAYN | 52.53625789 | 1.268013443 | 5.637401785 |
| KRT86 | 90.02109042 | 0.77714719 | 21.05319575 |
| STMN1 | 171.0376579 | 79.23568095 | 93.6313875 |
| ENTPD1 | 51.02960526 | 4.813320905 | 8.00948545 |
| KLRC2 | 170.3632789 | 27.43117567 | 71.7629798 |
| TOX | 111.2167158 | 40.17317381 | 48.47420821 |
| TNS3 | 18.24837105 | 2.614606619 | 1.99195922 |
| SRGAP3 | 21.03388737 | 0.327200095 | 6.54159999 |
| CLNK | 48.79801947 | 0.025489571 | 37.81930265 |
| AFAP1L2 | 48.02176947 | 0.917768462 | 5.888400985 |
| AKAP5 | 12.78739474 | 0.936769381 | 2.15521775 |
| CXCL13 | 1830.654842 | 11.14535171 | 33.24851965 |
| HAVCR2 | 334.6920737 | 43.65628381 | 59.708044 |
| KIR2DL4 | 90.73159421 | 3.772644619 | 20.33869545 |
| CHRM3-AS2 | 45.31885842 | 12.42002929 | 16.6765635 |
| TNFRSF9 | 75.16161053 | 13.75290395 | 7.85170705 |
| RRM2 | 51.55875105 | 5.512094643 | 12.54809355 |
| CD109 | 4.155530368 | 1.260725819 | 2.672376735 |
| DBH-AS1 | 9.190445 | 0 | 2.82368685 |
| SIRPG | 477.9031579 | 133.5109523 | 86.54938765 |
| CCL3 | 879.4891053 | 379.4398524 | 308.7706215 |
| KIF2C | 15.13782305 | 0.463829524 | 1.61216448 |
| CTLA4 | 243.8928526 | 19.54678733 | 68.7486734 |
| IFITM10 | 11.72409947 | 2.392565238 | 3.45431005 |
| GEM | 25.92595789 | 2.124119605 | 1.49090195 |
| RAD51AP1 | 17.47792795 | 6.90577151 | 4.517111 |
| KIFC1 | 2.437878232 | 1.01071879 | 1.177614 |
| NUSAP1 | 89.48275263 | 20.39847528 | 33.052021 |
| AURKA | 26.20345458 | 5.976808429 | 25.7213712 |
| VDR | 10.44611358 | 5.016252971 | 2.20061547 |
| KIAA0101 | 57.27524105 | 6.675146857 | 25.12704715 |
| ZC3H12C | 1.357966053 | 0.011761848 | 0.03016842 |
| ZBED2 | 44.29435947 | 0.845607048 | 3.18071175 |
| LILRP2 | 8.555959895 | 0.179393571 | 2.0487929 |
| FANCI | 18.30659889 | 7.114525952 | 7.359242145 |
| TNFSF4 | 41.49277777 | 2.741550938 | 6.68748513 |
| ASB2 | 43.76039474 | 4.745831905 | 9.542647915 |
| CAMK1 | 54.144063 | 7.5266972 | 21.17401165 |
| ANKS1B | 2.183310789 | 0.070079129 | 0.13357678 |
| SUOX | 16.12955742 | 4.120592919 | 2.128606905 |
| KCNK5 | 20.51417421 | 1.4114012 | 5.010913685 |
| KIF14 | 3.265626621 | 1.26698571 | 0.191578765 |
| SYNGR3 | 42.42283158 | 0.91940919 | 13.9666646 |
| C1orf106 | 2.195005284 | 1.078111543 | 0.415545935 |
| CCNA2 | 30.29312263 | 5.956798548 | 3.153623335 |
| CCRL2 | 41.17150789 | 17.92357785 | 15.53568475 |
| GINS1 | 9.327755947 | 1.102519233 | 1.842860195 |
| TK1 | 62.27577368 | 26.89102266 | 37.6692809 |
| BIRC5 | 21.82443158 | 9.183895781 | 17.58911704 |
| CASC5 | 6.408608889 | 0.954572352 | 1.822563205 |
| INPP5F | 14.65299721 | 0.683434986 | 6.52550963 |
| MCM2 | 34.84616211 | 9.475945462 | 13.86870412 |
| HJURP | 10.34542453 | 1.195678052 | 2.683584425 |
| RDH10 | 13.67609332 | 6.140745857 | 4.51163387 |
| FUT8 | 39.12060211 | 14.37280957 | 18.70667616 |
| MKI67 | 9.95709 | 0.396551648 | 2.29143949 |
| MYO1E | 15.26436368 | 1.932618633 | 2.655199115 |
| TOP2A | 32.63963384 | 6.18349561 | 16.73250851 |
| NDFIP2 | 91.40315263 | 16.69332039 | 39.17467275 |
| PDE7B | 4.094892895 | 0.369573876 | 0.69439915 |
| CDC6 | 6.325555684 | 0.878381595 | 1.80925055 |
| LOC1019289 | 13.66175105 | 1.286569143 | 5.42855735 |
| TNFRSF18 | 73.40014211 | 16.56000143 | 18.60834935 |
| WIPF3 | 18.05181664 | 1.792744448 | 1.021894 |
| CKAP2L | 7.193064868 | 0.582763748 | 2.08007515 |
| AMZ1 | 4.697318947 | 0.071634724 | 1.16409654 |
| ITGA2 | 5.027070905 | 0.309187614 | 1.389590445 |
| MZB1 | 56.75498526 | 9.430024524 | 16.1069718 |
| VCAM1 | 77.7627 | 12.47772077 | 20.12920756 |
| TIAM2 | 3.874846842 | 0.695292919 | 1.160521885 |
| SLC27A2 | 74.47079316 | 7.761453905 | 26.6987376 |
| UHRF1 | 9.493753579 | 2.803213095 | 2.78529736 |
| RIC1 | 9.940590942 | 4.073464 | 2.943654765 |
| EMC9 | 55.71777368 | 30.20844119 | 20.39791095 |
| CDK1 | 34.75512211 | 7.678148262 | 40.57957299 |
| PLAC8 | 10.19808989 | 114.6815 | 26.4785965 |
| FCGR3A | 14.86318363 | 357.0966381 | 74.9510268 |
| KLRF1 | 3.035728737 | 193.5823511 | 14.4933614 |
| DHRS3 | 53.74266979 | 222.4045952 | 143.5919088 |
| FCGR3B | 1.389735158 | 28.66079204 | 24.70543001 |
| CXCL16 | 13.30089653 | 175.1718914 | 85.036884 |
| LYZ | 197.5742774 | 2101.34619 | 1035.50759 |
| SVIL | 0.869931489 | 9.580544286 | 4.22403719 |
| PXN | 19.01642169 | 126.245899 | 71.63926645 |
| CRIP2 | 1.230886568 | 22.21384857 | 23.8480978 |
| CCL18 | 32.81444384 | 265.5379826 | 200.3426554 |
| C1QC | 59.72089795 | 365.8164711 | 164.5710445 |
| C1orf162 | 55.57553158 | 321.1258619 | 216.6321875 |
| PLAUR | 20.07343137 | 271.6738353 | 50.50656925 |
| TYROBP | 111.0874333 | 449.8064571 | 378.0819685 |
| | MeanTPM_ | | |
| | TIL_CD103neg | Min.log2FC | padj_Min |
| GSG2 | 0.43646014 | 1.727403278 | 0.017682536 |
| MYO7A | 2.571285816 | 2.259998486 | 0.000734598 |
| LAYN | 1.848024708 | 3.621535741 | 1.47E−13 |
| KRT86 | 3.71512388 | 2.036854575 | 0.020077613 |
| STMN1 | 85.79118 | 1.315548427 | 0.016592582 |
| ENTPD1 | 9.068538 | 2.51222904 | 7.94E−12 |
| KLRC2 | 42.90052344 | 1.140028446 | 0.040436251 |
| TOX | 52.981492 | 1.04690617 | 1.20E−09 |
| TNS3 | 1.578477292 | 2.871610018 | 5.17E−09 |
| SRGAP3 | 1.467354292 | 2.092844412 | 0.004201487 |
| CLNK | 3.97429732 | 1.421442605 | 0.034357992 |
| AFAP1L2 | 10.19192002 | 2.730768148 | 7.35E−09 |
| AKAP5 | 3.498434 | 2.119777167 | 2.15E−08 |
| CXCL13 | 200.4329206 | 2.786215532 | 2.15E−08 |
| HAVCR2 | 71.16694344 | 2.115630958 | 2.22E−08 |
| KIR2DL4 | 6.891746048 | 2.424076288 | 0.003617679 |
| CHRM3-AS2 | 2.12001284 | 2.363451642 | 0.003597258 |
| TNFRSF9 | 17.28012864 | 2.018972638 | 1.35E−07 |
| RRM2 | 24.39809328 | 2.032567257 | 8.03E−07 |
| CD109 | 1.058001288 | 1.712328303 | 0.00737706 |
| DBH-AS1 | 1.18816808 | 2.112220802 | 0.035666244 |
| SIRPG | 185.4196008 | 1.173047291 | 2.24E−06 |
| CCL3 | 448.815904 | 1.308047955 | 3.37E−06 |
| KIF2C | 2.716626244 | 2.340084386 | 4.65E−06 |
| CTLA4 | 50.97031992 | 2.049857964 | 3.76E−05 |
| IFITM10 | 1.272724276 | 2.259689528 | 5.64E−05 |
| GEM | 2.765744876 | 2.209029285 | 0.000114883 |
| RAD51AP1 | 2.22739396 | 2.090366569 | 0.015324702 |
| KIFC1 | 1.139757476 | 2.048040416 | 0.000116485 |
| NUSAP1 | 50.15568704 | 1.471883402 | 0.000146458 |
| AURKA | 17.55234268 | 1.660078116 | 0.00018727 |
| VDR | 3.135480952 | 1.916130686 | 0.000204746 |
| KIAA0101 | 34.92798628 | 1.705284926 | 0.000467234 |
| ZC3H12C | 0.060009268 | 2.032896561 | 0.000578224 |
| ZBED2 | 8.41407728 | 1.958522644 | 0.000775184 |
| LILRP2 | 0.24319804 | 1.929179014 | 0.00115957 |
| FANCI | 5.771166696 | 1.767956945 | 0.001462592 |
| TNFSF4 | 8.7365456 | 1.864855811 | 0.001607152 |
| ASB2 | 13.9961398 | 1.517671152 | 0.00161221 |
| CAMK1 | 12.76232984 | 1.691816551 | 0.00313345 |
| ANKS1B | 0.373814604 | 1.817118618 | 0.003144996 |
| SUOX | 2.148762428 | 1.791011507 | 0.003317961 |
| KCNK5 | 3.473615356 | 1.782686156 | 0.003491967 |
| KIF14 | 0.70509062 | 1.61077793 | 0.003902254 |
| SYNGR3 | 10.13336744 | 1.663443022 | 0.005290731 |
| C1orf106 | 0.429621136 | 1.654127927 | 0.005493609 |
| CCNA2 | 16.76836842 | 1.63474981 | 0.006225719 |
| CCRL2 | 18.98001112 | 1.61789572 | 0.006388745 |
| GINS1 | 2.35536462 | 1.561048491 | 0.006694195 |
| TK1 | 34.81022376 | 1.556990809 | 0.007377908 |
| BIRC5 | 17.88640092 | 1.52789537 | 0.007475706 |
| CASC5 | 0.7152126 | 1.683852608 | 0.008379571 |
| INPP5F | 4.56110998 | 1.557089828 | 0.010295127 |
| MCM2 | 12.7239374 | 1.417554569 | 0.013573646 |
| HJURP | 3.0432999 | 1.512082809 | 0.013662027 |
| RDH10 | 4.7784252 | 1.392983915 | 0.014000421 |
| FUT8 | 16.45712298 | 1.270544325 | 0.014375073 |
| MKI67 | 3.740788684 | 1.482800913 | 0.01475575 |
| MYO1E | 3.620583316 | 1.582425887 | 0.016029436 |
| TOP2A | 14.67442033 | 1.352846874 | 0.016288455 |
| NDFIP2 | 40.7288212 | 1.116525313 | 0.016478506 |
| PDE7B | 1.530637224 | 1.258502263 | 0.023019759 |
| CDC6 | 4.7555254 | 1.282112236 | 0.028458522 |
| LOC1019289 | 5.24736404 | 1.400426941 | 0.028458522 |
| TNFRSF18 | 20.86098484 | 1.436215773 | 0.031578469 |
| WIPF3 | 4.911925736 | 1.394904839 | 0.033969958 |
| CKAP2L | 3.42151756 | 1.321005047 | 0.036776193 |
| AMZ1 | 0.757580336 | 1.401084929 | 0.036835065 |
| ITGA2 | 3.19618754 | 1.216143898 | 0.04036681 |
| MZB1 | 24.83474744 | 1.309894553 | 0.041547254 |
| VCAM1 | 44.44741996 | 1.142380045 | 0.041909455 |
| TIAM2 | 0.52895268 | 1.421865316 | 0.042203047 |
| SLC27A2 | 31.71734129 | 1.267279008 | 0.042334312 |
| UHRF1 | 4.08198472 | 1.171771134 | 0.046651354 |
| RIC1 | 4.893008408 | 1.123345605 | 0.048428633 |
| EMC9 | 29.22870432 | 1.162931851 | 0.01302164 |
| CDK1 | 15.2390608 | 1.312177938 | 0.049332993 |
| PLAC8 | 55.3151272 | −1.464685708 | 0.037207354 |
| FCGR3A | 119.760642 | −2.930611162 | 8.59E−11 |
| KLRF1 | 69.15205336 | −2.393123248 | 1.41E−05 |
| DHRS3 | 150.522892 | −1.538676923 | 3.52E−05 |
| FCGR3B | 15.66373078 | −2.089580696 | 4.77E−05 |
| CXCL16 | 33.54755604 | −1.465177681 | 0.000968842 |
| LYZ | 533.8098288 | −1.206783578 | 0.0023407 |
| SVIL | 6.2219778 | −1.730549769 | 0.002803752 |
| PXN | 60.2272196 | −1.349346038 | 0.006403473 |
| CRIP2 | 10.84884325 | −1.682549856 | 0.007502923 |
| CCL18 | 120.7602916 | −1.43447936 | 0.023344239 |
| C1QC | 122.636079 | −1.289800192 | 0.025776769 |
| C1orf162 | 149.498455 | −1.321986663 | 0.029224437 |
| PLAUR | 88.264021 | −1.41499774 | 0.033969958 |
| TYROBP | 286.6825624 | −1.130633801 | 0.046142286 |

TABLE 6
Mapping metics obtained from MIGIC analysis

| | | | | | | Estimate |
|---|---|---|---|---|---|---|
| | | | | | | SAMPLE |
| | Name | Number | Sample | Class | Marker | TYPE |
| 1 | 12-TL647-TIL-CD8+_CD103+ | 12 | TL647 | TIL | CD8+_CD103+ | paired |
| 2 | 13-TL647-TIL-CD8+_CD103− | 13 | TL647 | TIL | CD8+_CD103− | paired |
| 3 | 139-TL706-TIL-CD8+_CD103+ | 139 | TL706 | TIL | CD8+_CD103+ | paired |
| 4 | 140-TL706-TIL-CD8+_CD103− | 140 | TL706 | TIL | CD8+_CD103− | paired |
| 5 | 151-TL722-TIL-CD8+_CD103+ | 151 | TL722 | TIL | CD8+_CD103+ | paired |
| 6 | 152-TL722-TIL-CD8+_CD103− | 152 | TL722 | TIL | CD8+_CD103− | paired |
| 7 | 157-TL704-TIL-CD8+_CD103+ | 157 | TL704 | TIL | CD8+_CD103+ | paired |
| 8 | 158-TL704-TIL-CD8+_CD103− | 158 | TL704 | TIL | CD8+_CD103− | paired |
| 9 | 172-TL720-TIL-CD8+_CD103+ | 172 | TL720 | TIL | CD8+_CD103+ | paired |
| 10 | 173-TL720-TIL-CD8+_CD103− | 173 | TL720 | TIL | CD8+_CD103− | paired |
| 11 | 18-TL615-TIL-CD8+_CD103+ | 18 | TL615 | TIL | CD8+_CD103+ | paired |
| 12 | 19-TL615-TIL-CD8+_CD103− | 19 | TL615 | TIL | CD8+_CD103− | paired |
| 13 | 55-TL661-TIL-CD8+_CD103+ | 55 | TL661 | TIL | CD8+_CD103+ | paired |
| 14 | 56-TL661-TIL-CD8+_CD103− | 56 | TL661 | TIL | CD8+_CD103− | paired |
| 15 | 63-TL663-TIL-CD8+_CD103+ | 63 | TL663 | TIL | CD8+_CD103+ | paired |
| 16 | 64-TL663-TIL-CD8+_CD103− | 64 | TL663 | TIL | CD8+_CD103− | paired |
| 17 | 90-TL101-TIL-CD8+_CD103+ | 90 | TL101 | TIL | CD8+_CD103+ | paired |
| 18 | 91-TL101-TIL-CD8+_CD103− | 91 | TL101 | TIL | CD8+_CD103− | paired |
| 19 | 95-TL684-TIL-CD8+_CD103+ | 95 | TL684 | TIL | CD8+_CD103+ | paired |
| 20 | 96-TL684-TIL-CD8+_CD103− | 96 | TL684 | TIL | CD8+_CD103− | paired |

| | | Estimate | Estimate | Estimate | Estimate | Estimate |
|---|---|---|---|---|---|---|
| | | TOTAL | TOTAL | OVERSEQ | COLLISION | UMI QUAL |
| | Name | READS | MIGS | THRESHOLD | THRESHOLD | THRESHOLD |
| 1 | 12-TL647-TIL-CD8+_CD103+ | 256397 | 3483 | 16 | 16 | 15 |
| 2 | 13-TL647-TIL-CD8+_CD103− | 105892 | 1787 | 16 | 16 | 15 |
| 3 | 139-TL706-TIL-CD8+_CD103+ | 157923 | 3661 | 11 | 11 | 15 |
| 4 | 140-TL706-TIL-CD8+_CD103− | 414797 | 4264 | 23 | 23 | 15 |
| 5 | 151-TL722-TIL-CD8+_CD103+ | 144130 | 3141 | 11 | 11 | 15 |
| 6 | 152-TL722-TIL-CD8+_CD103− | 193457 | 1743 | 32 | 32 | 15 |
| 7 | 157-TL704-TIL-CD8+_CD103+ | 242088 | 3713 | 11 | 11 | 15 |
| 8 | 158-TL704-TIL-CD8+_CD103− | 228663 | 3040 | 16 | 16 | 15 |
| 9 | 172-TL720-TIL-CD8+_CD103+ | 185525 | 1773 | 32 | 32 | 15 |
| 10 | 173-TL720-TIL-CD8+_CD103− | 158541 | 1973 | 16 | 16 | 15 |
| 11 | 18-TL615-TIL-CD8+_CD103+ | 230107 | 4147 | 11 | 11 | 15 |
| 12 | 19-TL615-TIL-CD8+_CD103− | 294826 | 3764 | 16 | 16 | 15 |
| 13 | 55-TL661-TIL-CD8+_CD103+ | 179352 | 2788 | 16 | 16 | 15 |
| 14 | 56-TL661-TIL-CD8+_CD103− | 62968 | 1385 | 11 | 11 | 15 |
| 15 | 63-TL663-TIL-CD8+_CD103+ | 262129 | 4085 | 16 | 16 | 15 |
| 16 | 64-TL663-TIL-CD8+_CD103− | 261288 | 3438 | 23 | 23 | 15 |
| 17 | 90-TL101-TIL-CD8+_CD103+ | 125051 | 1037 | 32 | 32 | 15 |
| 18 | 91-TL101-TIL-CD8+_CD103− | 65514 | 2602 | 6 | 6 | 15 |
| 19 | 95-TL684-TIL-CD8+_CD103+ | 290295 | 2234 | 32 | 32 | 15 |
| 20 | 96-TL684-TIL-CD8+_CD103− | 167628 | 1297 | 32 | 32 | 15 |

| | | | Assemble | Assemble | Assemble | Assemble |
|---|---|---|---|---|---|---|
| | | Estimate | MIG COUNT | MIGS GOOD | MIGS GOOD | MIGS GOOD |
| | Name | UMI LEN | THRESHOLD | FASTQ1 | FASTQ2 | TOTAL |
| 1 | 12-TL647-TIL-CD8+_CD103+ | 12 | 16 | 881 | 886 | 874 |
| 2 | 13-TL647-TIL-CD8+_CD103− | 12 | 16 | 317 | 317 | 313 |
| 3 | 139-TL706-TIL-CD8+_CD103+ | 12 | 11 | 1548 | 1555 | 1540 |
| 4 | 140-TL706-TIL-CD8+_CD103− | 12 | 23 | 743 | 745 | 741 |
| 5 | 151-TL722-TIL-CD8+_CD103+ | 12 | 11 | 1346 | 1344 | 1329 |
| 6 | 152-TL722-TIL-CD8+_CD103− | 12 | 32 | 152 | 155 | 150 |
| 7 | 157-TL704-TIL-CD8+_CD103+ | 12 | 11 | 1489 | 1491 | 1483 |
| 8 | 158-TL704-TIL-CD8+_CD103− | 12 | 16 | 728 | 727 | 716 |
| 9 | 172-TL720-TIL-CD8+_CD103+ | 12 | 32 | 219 | 215 | 214 |
| 10 | 173-TL720-TIL-CD8+_CD103− | 12 | 16 | 524 | 526 | 523 |
| 11 | 18-TL615-TIL-CD8+_CD103+ | 12 | 11 | 1488 | 1470 | 1458 |
| 12 | 19-TL615-TIL-CD8+_CD103− | 12 | 16 | 906 | 905 | 895 |
| 13 | 55-TL661-TIL-CD8+_CD103+ | 12 | 16 | 578 | 581 | 573 |
| 14 | 56-TL661-TIL-CD8+_CD103− | 12 | 11 | 310 | 310 | 308 |
| 15 | 63-TL663-TIL-CD8+_CD103+ | 12 | 16 | 720 | 724 | 717 |
| 16 | 64-TL663-TIL-CD8+_CD103− | 12 | 23 | 511 | 518 | 510 |
| 17 | 90-TL101-TIL-CD8+_CD103+ | 12 | 32 | 187 | 188 | 186 |
| 18 | 91-TL101-TIL-CD8+_CD103− | 12 | 6 | 1704 | 1705 | 1682 |
| 19 | 95-TL684-TIL-CD8+_CD103+ | 12 | 32 | 276 | 275 | 275 |
| 20 | 96-TL684-TIL-CD8+_CD103− | 12 | 32 | 180 | 180 | 180 |

| | | | Assemble | Assemble | Assemble | |
|---|---|---|---|---|---|---|
| | | Assemble | READS | READS | READS | Assemble |
| | | MIGS | GOOD | GOOD | GOOD | READS |
| | Name | TOTAL | FASTQ1 | FASTQ2 | TOTAL | TOTAL |
| 1 | 12-TL647-TIL-CD8+_CD103+ | 3483 | 215978 | 224093 | 235916 | 242518 |
| 2 | 13-TL647-TIL-CD8+_CD103− | 1787 | 90486 | 93526 | 94556 | 99609 |
| 3 | 139-TL706-TIL-CD8+_CD103+ | 3661 | 137863 | 140542 | 144314 | 148820 |
| 4 | 140-TL706-TIL-CD8+_CD103− | 4264 | 361751 | 373821 | 382965 | 389088 |
| 5 | 151-TL722-TIL-CD8+_CD103+ | 3141 | 125468 | 129630 | 131603 | 136224 |
| 6 | 152-TL722-TIL-CD8+_CD103− | 1743 | 170625 | 174451 | 176010 | 181048 |
| 7 | 157-TL704-TIL-CD8+_CD103+ | 3713 | 213284 | 216903 | 224884 | 229289 |
| 8 | 158-TL704-TIL-CD8+_CD103− | 3040 | 196480 | 205688 | 209293 | 215321 |
| 9 | 172-TL720-TIL-CD8+_CD103+ | 1773 | 163398 | 169622 | 171133 | 175191 |
| 10 | 173-TL720-TIL-CD8+_CD103− | 1973 | 141013 | 145563 | 146520 | 148966 |
| 11 | 18-TL615-TIL-CD8+_CD103+ | 4147 | 200782 | 205033 | 210574 | 216662 |
| 12 | 19-TL615-TIL-CD8+_CD103− | 3764 | 256428 | 265348 | 269313 | 277718 |
| 13 | 55-TL661-TIL-CD8+_CD103+ | 2788 | 154888 | 158301 | 161188 | 167841 |
| 14 | 56-TL661-TIL-CD8+_CD103− | 1385 | 53932 | 55422 | 55888 | 58920 |
| 15 | 63-TL663-TIL-CD8+_CD103+ | 4085 | 225440 | 234365 | 236938 | 246198 |
| 16 | 64-TL663-TIL-CD8+_CD103− | 3438 | 221547 | 233768 | 235752 | 245422 |
| 17 | 90-TL101-TIL-CD8+_CD103+ | 1037 | 108119 | 114592 | 115402 | 117838 |
| 18 | 91-TL101-TIL-CD8+_CD103− | 2602 | 56311 | 57744 | 59538 | 61447 |
| 19 | 95-TL684-TIL-CD8+_CD103+ | 2234 | 255442 | 265674 | 268247 | 271670 |
| 20 | 96-TL684-TIL-CD8+_CD103− | 1297 | 145259 | 154277 | 156186 | 158039 |

| | | Assemble | | | | |
|---|---|---|---|---|---|---|
| | | READS | | | | |
| | | DROPPED | CDRBlast | CDRBlast | CDRBlast | CDRBlast |
| | | WITHIN | DATA | EVENTS | EVENTS | EVENTS |
| | Name | MIG | TYPE | GOOD | MAPPED | TOTAL |
| 1 | 12-TL647-TIL-CD8+_CD103+ | 32002 | asm | 699 | 741 | 1748 |
| 2 | 13-TL647-TIL-CD8+_CD103− | 6573 | asm | 259 | 263 | 626 |
| 3 | 139-TL706-TIL-CD8+_CD103+ | 10968 | asm | 1068 | 1183 | 3080 |
| 4 | 140-TL706-TIL-CD8+_CD103− | 30862 | asm | 494 | 519 | 1482 |
| 5 | 151-TL722-TIL-CD8+_CD103+ | 9686 | asm | 929 | 1054 | 2658 |
| 6 | 152-TL722-TIL-CD8+_CD103− | 6825 | asm | 90 | 95 | 300 |
| 7 | 157-TL704-TIL-CD8+_CD103+ | 20961 | asm | 1054 | 1161 | 2966 |
| 8 | 158-TL704-TIL-CD8+_CD103− | 17696 | asm | 592 | 652 | 1432 |
| 9 | 172-TL720-TIL-CD8+_CD103+ | 8793 | asm | 150 | 164 | 428 |
| 10 | 173-TL720-TIL-CD8+_CD103− | 6904 | asm | 407 | 429 | 1046 |
| 11 | 18-TL615-TIL-CD8+_CD103+ | 16414 | asm | 1139 | 1277 | 2916 |
| 12 | 19-TL615-TIL-CD8+_CD103− | 19363 | asm | 731 | 780 | 1790 |
| 13 | 55-TL661-TIL-CD8+_CD103+ | 10405 | asm | 412 | 422 | 1146 |
| 14 | 56-TL661-TIL-CD8+_CD103− | 2553 | asm | 236 | 244 | 616 |
| 15 | 63-TL663-TIL-CD8+_CD103+ | 15508 | asm | 572 | 582 | 1434 |
| 16 | 64-TL663-TIL-CD8+_CD103− | 17675 | asm | 374 | 377 | 1020 |
| 17 | 90-TL101-TIL-CD8+_CD103+ | 9472 | asm | 123 | 123 | 372 |
| 18 | 91-TL101-TIL-CD8+_CD103− | 5835 | asm | 1167 | 1455 | 3364 |
| 19 | 95-TL684-TIL-CD8+_CD103+ | 14558 | asm | 202 | 203 | 550 |
| 20 | 96-TL684-TIL-CD8+_CD103− | 10653 | asm | 136 | 136 | 360 |

| | | CDRBlast | CDRBlast | CDRBlast | Number | Number |
|---|---|---|---|---|---|---|
| | | READS | READS | READS | TCR | Clonotypes |
| | Name | GOOD | MAPPED | TOTAL | molecules | Found |
| 1 | 12-TL647-TIL-CD8+_CD103+ | 175402 | 177879 | 439744 | 654 | 99 |
| 2 | 13-TL647-TIL-CD8+_CD103− | 77762 | 77873 | 183383 | 237 | 67 |
| 3 | 139-TL706-TIL-CD8+_CD103+ | 87825 | 93692 | 278009 | 974 | 157 |
| 4 | 140-TL706-TIL-CD8+_CD103− | 212346 | 231002 | 734848 | 472 | 175 |
| 5 | 151-TL722-TIL-CD8+_CD103+ | 83161 | 92665 | 254326 | 878 | 117 |
| 6 | 152-TL722-TIL-CD8+_CD103− | 104254 | 104440 | 344846 | 85 | 60 |
| 7 | 157-TL704-TIL-CD8+_CD103+ | 119636 | 128407 | 428990 | 998 | 68 |
| 8 | 158-TL704-TIL-CD8+_CD103− | 154956 | 162679 | 401324 | 558 | 224 |
| 9 | 172-TL720-TIL-CD8+_CD103+ | 119858 | 120386 | 332822 | 143 | 85 |
| 10 | 173-TL720-TIL-CD8+_CD103− | 114808 | 115925 | 286238 | 380 | 146 |
| 11 | 18-TL615-TIL-CD8+_CD103+ | 160660 | 168339 | 405084 | 1053 | 137 |
| 12 | 19-TL615-TIL-CD8+_CD103− | 226581 | 228568 | 520271 | 674 | 192 |
| 13 | 55-TL661-TIL-CD8+_CD103+ | 122895 | 124004 | 312583 | 387 | 108 |
| 14 | 56-TL661-TIL-CD8+_CD103− | 43713 | 43813 | 109221 | 216 | 111 |
| 15 | 63-TL663-TIL-CD8+_CD103+ | 178541 | 182139 | 458777 | 550 | 163 |
| 16 | 64-TL663-TIL-CD8+_CD103− | 166911 | 168724 | 453958 | 350 | 171 |
| 17 | 90-TL101-TIL-CD8+_CD103+ | 53875 | 53875 | 222323 | 118 | 47 |
| 18 | 91-TL101-TIL-CD8+_CD103− | 40252 | 46213 | 113574 | 1047 | 653 |
| 19 | 95-TL684-TIL-CD8+_CD103+ | 202918 | 202966 | 520997 | 190 | 103 |
| 20 | 96-TL684-TIL-CD8+_CD103− | 104038 | 104038 | 299536 | 129 | 92 |

| Sample ID | Sample | Class | Marker | Filter |
|---|---|---|---|---|
| 12-TL647-TIL-CD8+_CD103+ | TL647 | TIL | CD8+_CD103+ | conv:MiGec |
| 13-TL647-TIL-CD8+_CD103− | TL647 | TIL | CD8+_CD103− | conv:MiGec |
| 139-TL706-TIL-CD8+_CD103+ | TL706 | TIL | CD8+_CD103+ | conv:MiGec |
| 140-TL706-TIL-CD8+_CD103− | TL706 | TIL | CD8+_CD103− | conv:MiGec |
| 151-TL722-TIL-CD8+_CD103+ | TL722 | TIL | CD8+_CD103+ | conv:MiGec |
| 152-TL722-TIL-CD8+_CD103− | TL722 | TIL | CD8+_CD103− | conv:MiGec |
| 157-TL704-TIL-CD8+_CD103+ | TL704 | TIL | CD8+_CD103+ | conv:MiGec |
| 158-TL704-TIL-CD8+_CD103− | TL704 | TIL | CD8+_CD103− | conv:MiGec |
| 172-TL720-TIL-CD8+_CD103+ | TL720 | TIL | CD8+_CD103+ | conv:MiGec |
| 173-TL720-TIL-CD8+_CD103− | TL720 | TIL | CD8+_CD103− | conv:MiGec |
| 18-TL615-TIL-CD8+_CD103+ | TL615 | TIL | CD8+_CD103+ | conv:MiGec |
| 19-TL615-TIL-CD8+_CD103− | TL615 | TIL | CD8+_CD103− | conv:MiGec |
| 55-TL661-TIL-CD8+_CD103+ | TL661 | TIL | CD8+_CD103+ | conv:MiGec |
| 56-TL661-TIL-CD8+_CD103− | TL661 | TIL | CD8+_CD103− | conv:MiGec |
| 63-TL663-TIL-CD8+_CD103+ | TL663 | TIL | CD8+_CD103+ | conv:MiGec |
| 64-TL663-TIL-CD8+_CD103− | TL663 | TIL | CD8+_CD103− | conv:MiGec |
| 90-TL101-TIL-CD8+_CD103+ | TL101 | TIL | CD8+_CD103+ | conv:MiGec |
| 91-TL101-TIL-CD8+_CD103− | TL101 | TIL | CD8+_CD103− | conv:MiGec |
| 95-TL684-TIL-CD8+_CD103+ | TL684 | TIL | CD8+_CD103+ | conv:MiGec |
| 96-TL684-TIL-CD8+_CD103− | TL684 | TIL | CD8+_CD103− | conv:MiGec |

| | | | Extrapolate | Chao1 |
|---|---|---|---|---|
| Sample ID | Read | Diversity | reads | mean |
| 12-TL647-TIL-CD8+_CD103+ | 654 | 99 | 1053 | 235 |
| 13-TL647-TIL-CD8+_CD103− | 237 | 67 | 1053 | 166 |
| 139-TL706-TIL-CD8+_CD103+ | 974 | 157 | 1053 | 265 |
| 140-TL706-TIL-CD8+_CD103− | 472 | 175 | 1053 | 451 |
| 151-TL722-TIL-CD8+_CD103+ | 878 | 117 | 1053 | 203 |
| 152-TL722-TIL-CD8+ CD103− | 85 | 60 | 1053 | 163 |
| 157-TL704-TIL-CD8+_CD103+ | 998 | 68 | 1053 | 103 |
| 158-TL704-TIL-CD8+_CD103− | 558 | 224 | 1053 | 750 |
| 172-TL720-TIL-CD8+_CD103+ | 143 | 85 | 1053 | 269 |
| 173-TL720-TIL-CD8+_CD103− | 380 | 146 | 1053 | 344 |
| 18-TL615-TIL-CD8+_CD103+ | 1053 | 137 | 1053 | 252 |
| 19-TL615-TIL-CD8+_CD103− | 674 | 192 | 1053 | 446 |
| 55-TL661-TIL-CD8+_CD103+ | 387 | 108 | 1053 | 165 |
| 56-TL661-TIL-CD8+_CD103− | 216 | 111 | 1053 | 274 |
| 63-TL663-TIL-CD8+_CD103+ | 550 | 163 | 1053 | 326 |
| 64-TL663-TIL-CD8+_CD103− | 350 | 171 | 1053 | 473 |
| 90-TL101-TIL-CD8+_CD103+ | 118 | 47 | 1053 | 152 |
| 91-TL101-TIL-CD8+_CD103− | 1047 | 653 | 1053 | 1848 |
| 95-TL684-TIL-CD8+_CD103+ | 190 | 103 | 1053 | 259 |
| 96-TL684-TIL-CD8+_CD103− | 129 | 92 | 1053 | 262 |

| | ObservedDiversity | ChaoE | ChaoE | EfronThisted | EfronThisted |
|---|---|---|---|---|---|
| Sample ID | mean | mean | std | mean | std |
| 12-TL647-TIL-CD8+_CD103+ | 99 | 131 | 8 | 207 | 15 |
| 13-TL647-TIL-CD8+_CD103− | 67 | 145 | 23 | 112 | 6 |
| 139-TL706-TIL-CD8+_CD103+ | 157 | 163 | 8 | 295 | 19 |
| 140-TL706-TIL-CD8+_CD103− | 175 | 287 | 16 | 387 | 22 |
| 151-TL722-TIL-CD8+_CD103+ | 117 | 129 | 7 | 224 | 16 |
| 152-TL722-TIL-CD8+_CD103− | 60 | 162 | 41 | 106 | 6 |
| 157-TL704-TIL-CD8+_CD103+ | 68 | 69 | 4 | 95 | 5 |
| 158-TL704-TIL-CD8+_CD103− | 224 | 352 | 16 | 654 | 41 |
| 172-TL720-TIL-CD8+_CD103+ | 85 | 251 | 48 | 152 | 8 |
| 173-TL720-TIL-CD8+_CD103− | 146 | 261 | 19 | 319 | 20 |
| 18-TL615-TIL-CD8+_CD103+ | 137 | 137 | 7 | 268 | 18 |
| 19-TL615-TIL-CD8+_CD103− | 192 | 252 | 11 | 408 | 22 |
| 55-TL661-TIL-CD8+_CD103+ | 108 | 154 | 12 | 194 | 15 |
| 56-TL661-TIL-CD8+_CD103− | 111 | 246 | 32 | 245 | 17 |
| 63-TL663-TIL-CD8+_CD103+ | 163 | 232 | 12 | 331 | 20 |
| 64-TL663-TIL-CD8+_CD103− | 171 | 338 | 24 | 390 | 22 |
| 90-TL101-TIL-CD8+_CD103+ | 47 | 145 | 41 | 83 | 6 |
| 91-TL101-TIL-CD8+_CD103− | 653 | 655 | 20 | 2075 | 108 |
| 95-TL684-TIL-CD8+_CD103+ | 103 | 238 | 35 | 227 | 17 |
| 96-TL684-TIL-CD8+_CD103− | 92 | 254 | 48 | 164 | 8 |

| | | | | normalized | |
|---|---|---|---|---|---|
| | Chao1 | d50Index | shannonWienerIndex | ShannonWienerIndex | inverseSimpsonIndex |
| Sample ID | std | mean | mean | mean | mean |
| 12-TL647-TIL-CD8+_CD103+ | 52 | 0.95959596 | 20.53006238 | 0.6576303 | 10.35732274 |
| 13-TL647-TIL-CD8+_CD103− | 43 | 0.955223881 | 17.98794136 | 0.6872563 | 5.595079191 |
| 139-TL706-TIL-CD8+_CD103+ | 31 | 0.961783439 | 36.38352009 | 0.710827 | 16.06999356 |
| 140-TL706-TIL-CD8+_CD103− | 76 | 0.92 | 68.48994371 | 0.8183663 | 19.89142857 |
| 151-TL722-TIL-CD8+_CD103+ | 29 | 0.965811966 | 24.32970049 | 0.6702187 | 10.55629502 |
| 152-TL722-TIL-CD8+_CD103− | 44 | 0.9 | 51.00535901 | 0.9603321 | 40.81920904 |
| 157-TL704-TIL-CD8+_CD103+ | 18 | 0.911764706 | 21.09133591 | 0.7225635 | 11.21575605 |
| 158-TL704-TIL-CD8+_CD103− | 132 | 0.9375 | 93.37867751 | 0.8383148 | 33.0675446 |
| 172-TL720-TIL-CD8+_CD103+ | 69 | 0.929411765 | 53.57509948 | 0.8961055 | 25.72201258 |
| 173-TL720-TIL-CD8+_CD103− | 58 | 0.904109589 | 70.32517191 | 0.8534241 | 31.58355206 |
| 18-TL615-TIL-CD8+_CD103+ | 36 | 0.97080292 | 24.05847811 | 0.646443 | 9.060895786 |
| 19-TL615-TIL-CD8+_CD103− | 67 | 0.942708333 | 68.829179 | 0.8048752 | 29.23645257 |
| 55-TL661-TIL-CD8+_CD103+ | 20 | 0.944444444 | 37.173325 | 0.7722106 | 13.21296868 |
| 56-TL661-TIL-CD8+_CD103− | 55 | 0.90990991 | 77.24672227 | 0.923023 | 50.06008584 |
| 63-TL663-TIL-CD8+_CD103+ | 46 | 0.895705521 | 64.85979256 | 0.8190877 | 24.16520211 |
| 64-TL663-TIL-CD8+_CD103− | 83 | 0.941520468 | 98.46364678 | 0.8926464 | 43.01264045 |
| 90-TL101-TIL-CD8+_CD103+ | 53 | 0.936170213 | 19.01294967 | 0.764937 | 8.307875895 |
| 91-TL101-TIL-CD8+_CD103− | 165 | 0.977029096 | 485.3991287 | 0.9542387 | 299.920383 |
| 95-TL684-TIL-CD8+_CD103+ | 55 | 0.912621359 | 77.11035818 | 0.9375387 | 55.53846154 |
| 96-TL684-TIL-CD8+_CD103− | 60 | 0.923913043 | 76.75366646 | 0.9599301 | 57.9825784 |

| Name | Number | Sample | Class | Marker |
|---|---|---|---|---|
| 12-TL647-TIL-CD8+_CD103+ | 12 | TL647 | TIL | CD8+_CD103+ |
| 13-TL647-TIL-CD8+_CD103− | 13 | TL647 | TIL | CD8+_CD103− |
| 139-TL706-TIL-CD8+_CD103+ | 139 | TL706 | TIL | CD8+_CD103+ |
| 140-TL706-TIL-CD8+_CD103− | 140 | TL706 | TIL | CD8+_CD103− |
| 151-TL722-TIL-CD8+_CD103+ | 151 | TL722 | TIL | CD8+_CD103+ |
| 152-TL722-TIL-CD8+_CD103− | 152 | TL722 | TIL | CD8+_CD103− |
| 157-TL704-TIL-CD8+_CD103+ | 157 | TL704 | TIL | CD8+_CD103+ |
| 158-TL704-TIL-CD8+_CD103− | 158 | TL704 | TIL | CD8+_CD103− |
| 172-TL720-TIL-CD8+_CD103+ | 172 | TL720 | TIL | CD8+_CD103+ |
| 173-TL720-TIL-CD8+_CD103− | 173 | TL720 | TIL | CD8+_CD103− |
| 18-TL615-TIL-CD8+_CD103+ | 18 | TL615 | TIL | CD8+_CD103+ |
| 19-TL615-TIL-CD8+_CD103− | 19 | TL615 | TIL | CD8+_CD103− |
| 55-TL661-TIL-CD8+_CD103+ | 55 | TL661 | TIL | CD8+_CD103+ |
| 56-TL661-TIL-CD8+_CD103− | 56 | TL661 | TIL | CD8+_CD103− |
| 63-TL663-TIL-CD8+_CD103+ | 63 | TL663 | TIL | CD8+_CD103+ |
| 64-TL663-TIL-CD8+_CD103− | 64 | TL663 | TIL | CD8+_CD103− |
| 90-TL101-TIL-CD8+_CD103+ | 90 | TL101 | TIL | CD8+_CD103+ |
| 91-TL101-TIL-CD8+_CD103− | 91 | TL101 | TIL | CD8+_CD103− |
| 95-TL684-TIL-CD8+_CD103+ | 95 | TL684 | TIL | CD8+_CD103+ |
| 96-TL684-TIL-CD8+_CD103− | 96 | TL684 | TIL | CD8+_CD103− |

| | Percent top | Percent sec | Percent rem | Percent all | Percent non |
|---|---|---|---|---|---|
| Name | exp clone | exp clones | exp clones | exp clones | exp clones |
| 12-TL647-TIL-CD8+_CD103+ | 17 | 15 | 55 | 87 | 13 |
| 13-TL647-TIL-CD8+_CD103− | 41 | 8 | 25 | 73 | 27 |
| 139-TL706-TIL-CD8+_CD103+ | 16 | 10 | 59 | 85 | 15 |
| 140-TL706-TIL-CD8+_CD103− | 19 | 7 | 39 | 65 | 35 |
| 151-TL722-TIL-CD8+_CD103+ | 22 | 15 | 51 | 87 | 13 |
| 152-TL722-TIL-CD8+ CD103− | 7 | 6 | 12 | 25 | 75 |
| 157-TL704-TIL-CD8+_CD103+ | 24 | 9 | 63 | 95 | 5 |
| 158-TL704-TIL-CD8+_CD103− | 13 | 5 | 43 | 61 | 39 |
| 172-TL720-TIL-CD8+_CD103+ | 15 | 8 | 15 | 38 | 62 |
| 173-TL720-TIL-CD8+_CD103− | 11 | 8 | 43 | 62 | 38 |
| 18-TL615-TIL-CD8+_CD103+ | 27 | 11 | 49 | 88 | 12 |
| 19-TL615-TIL-CD8+_CD103− | 9 | 9 | 55 | 74 | 26 |
| 55-TL661-TIL-CD8+_CD103+ | 22 | 12 | 39 | 72 | 28 |
| 56-TL661-TIL-CD8+_CD103− | 7 | 6 | 38 | 50 | 50 |
| 63-TL663-TIL-CD8+_CD103+ | 16 | 6 | 49 | 72 | 28 |
| 64-TL663-TIL-CD8+_CD103− | 11 | 7 | 35 | 52 | 48 |
| 90-TL101-TIL-CD8+_CD103+ | 26 | 20 | 14 | 61 | 39 |
| 91-TL101-TIL-CD8+_CD103− | 2 | 2 | 31 | 35 | 65 |
| 95-TL684-TIL-CD8+_CD103+ | 5 | 8 | 35 | 49 | 51 |
| 96-TL684-TIL-CD8+_CD103− | 6 | 5 | 12 | 22 | 78 |

TCRβ chain reconstruction in subjects from TCR-seq analysis

| Number | Name | Class | Marker | CDR3 nucleotide sequence |
|---|---|---|---|---|
| 1 | 12-TL647-TIL-CD8+_CD103+ | TIL | CD8+_CD103+ | TGCAGCGTTGAAGGAGGGCGACTAGAGGCAGATACGCAGTATTTT |
| 2 | 12-TL647-TIL-CD8+_CD103+ | TIL | CD8+_CD103+ | TGTGCCAGCAGTCCCTCATCCCTTGGACAGGACAATCAGCCCCAGCATTTT |
| 3 | 12-TL647-TIL-CD8+_CD103+ | TIL | CD8+_CD103+ | TGTGCCAGCAGCTTAGGGTCAGGGGAGTACATTCAGTACTTC |
| 4 | 12-TL647-TIL-CD8+_CD103+ | TIL | CD8+_CD103+ | TGTGCCAGCAGCCCAGGGACTAGCTACATTCAGTTCTTC |
| 5 | 12-TL647-TIL-CD8+_CD103+ | TIL | CD8+_CD103+ | TGTGCCAGCAGCGTAGCTAGCGGGACAGATACGCAGTATTTT |
| 6 | 12-TL647-TIL-CD8+_CD103+ | TIL | CD8+_CD103+ | TGTGCCAGCAGTTTATCAAGGAGCCAGTCCTCTAAAGCTTTCTTT |
| 7 | 12-TL647-TIL-CD8+_CD103+ | TIL | CD8+_CD103+ | TGTGCCAGCAGCTCGACAGGGTACTATGGCTACACCTTC |
| 8 | 12-TL647-TIL-CD8+_CD103+ | TIL | CD8+_CD103+ | TGCGCCAGCAGCTCCGGACAGGGGGCCACTGAAGCTTTCTTT |
| 9 | 12-TL647-TIL-CD8+_CD103+ | TIL | CD8+_CD103+ | TGTGCCAGCAGCCCGGATAGCAATCAGCCCCAGCATTTT |
| 10 | 12-TL647-TIL-CD8+_CD103+ | TIL | CD8+_CD103+ | TGTGCCAGCAGCTTAGGAGGGGGCTTATACGAGCAGTACTTC |
| 11 | 12-TL647-TIL-CD8+_CD103+ | TIL | CD8+_CD103+ | TGTGCCAGCAGGACAGGGGGTGATGGCTACACCTTC |
| 12 | 12-TL647-TIL-CD8+_CD103+ | TIL | CD8+_CD103+ | TGTGCCAGCAGCTCCATAGCGGGGAGCTCCTACAATGAGCAGTTCTTC |
| 13 | 12-TL647-TIL-CD8+_CD103+ | TIL | CD8+_CD103+ | TGTGCCAGTAGTATAGGGCAGCAAGAGCAGTTCTTC |
| 14 | 12-TL647-TIL-CD8+_CD103+ | TIL | CD8+_CD103+ | TGCGCCAGTAGGACAGGCAATGAGCAGTTCTTC |
| 15 | 12-TL647-TIL-CD8+_CD103+ | TIL | CD8+_CD103+ | TGCGCCAGCAGCCCAGACTGGTTCCAGGTCTACGAGCAGTACTTC |
| 16 | 12-TL647-TIL-CD8+_CD103+ | TIL | CD8+_CD103+ | TGTGCCAGCAGCCCGGGGACTACATGGACGCAGTATTTT |
| 17 | 12-TL647-TIL-CD8+_CD103+ | TIL | CD8+_CD103+ | TGTGCCAGCAGCGAAAGAGGACTCACTGAAGCTTTCTTT |
| 18 | 12-TL647-TIL-CD8+_CD103+ | TIL | CD8+_CD103+ | TGCGCCAGCAGCTTGGAACCAGTAGGACCTTACAATGAGCAGTTCTTC |
| 19 | 12-TL647-TIL-CD8+_CD103+ | TIL | CD8+_CD103+ | TGCAGCGGCGGAGGGAGGTTAACGCAGTATTTT |
| 20 | 12-TL647-TIL-CD8+_CD103+ | TIL | CD8+_CD103+ | TGTGCTCTTGGGGACCCTAGCTCCGGGGAGCTGTTTTTT |
| 21 | 12-TL647-TIL-CD8+_CD103+ | TIL | CD8+_CD103+ | TGTGCCAGCACCCCCGATGGGGCGAATCAGTACTTC |
| 22 | 12-TL647-TIL-CD8+_CD103+ | TIL | CD8+_CD103+ | TGTGCCACCAGCATTACCGGGACAGGGAAACCCTACGAGCAGTACTTC |
| 23 | 12-TL647-TIL-CD8+_CD103+ | TIL | CD8+_CD103+ | TGTGCCAGCAGCTTCATGCGGCCGGGACAGGGGGCGGTGGGGGATTCA |
| | | | | CCCCTCCACTTT |
| 24 | 12-TL647-TIL-CD8+_CD103+ | TIL | CD8+_CD103+ | TGTGCCAGCAGTTTTCGTGGGCTTGGAGCTTTCTTT |
| 25 | 12-TL647-TIL-CD8+_CD103+ | TIL | CD8+_CD103+ | TGTGCCAGCAGCCCCCGACTAGCGGGGCTCTACGAGCAGTACTTC |
| 26 | 12-TL647-TIL-CD8+_CD103+ | TIL | CD8+_CD103+ | TGTGCCAGCAGCTTGCGGCTAGCGGGCGCCTCCCTTTACAATGAG |
| | | | | CAGTTCTTC |
| 27 | 12-TL647-TIL-CD8+_CD103+ | TIL | CD8+_CD103+ | TGTGCCAGCAGTCCTACAGGCCAAGAGACCCAGTACTTC |
| 28 | 12-TL647-TIL-CD8+_CD103+ | TIL | CD8+_CD103+ | TGCAGCGTTGAAGGGGGTCGGGGGCGGGGGGATACGCAGTATTTT |
| 29 | 12-TL647-TIL-CD8+_CD103+ | TIL | CD8+_CD103+ | TGCAGTGCTAGAGATGACCTTAAACCTGCCGAGCAGTACTTC |
| 30 | 12-TL647-TIL-CD8+_CD103+ | TIL | CD8+_CD103+ | TGTGCCAGCAGTGGGCTTTACTCAGATACGCAGTATTTT |
| 31 | 12-TL647-TIL-CD8+_CD103+ | TIL | CD8+_CD103+ | TGTGCCAGCAGCTTGGCCGGGACGTCCCATCAGCCCCAGCATTTT |
| 32 | 12-TL647-TIL-CD8+_CD103+ | TIL | CD8+_CD103+ | TGTGCCAGTACTACTCGACAGGGGGGTGTAAGAAATCAGCCCCAGCATTTT |
| 33 | 12-TL647-TIL-CD8+_CD103+ | TIL | CD8+_CD103+ | TGTGCCAGCAGCCAAGAGGGAACTAGCGCGACCTACGAGCAGTACTTC |
| 34 | 12-TL647-TIL-CD8+_CD103+ | TIL | CD8+_CD103+ | TGCAGTGCTAGTGGTTGGGACAGTAAATTCAATGAGCAGTTCTTC |
| 35 | 12-TL647-TIL-CD8+_CD103+ | TIL | CD8+_CD103+ | TGCGCCAGCAGTAGCAGGATCGGGGAGCTGTTTTTT |
| 36 | 12-TL647-TIL-CD8+_CD103+ | TIL | CD8+_CD103+ | TGTGCCAGCAGGGGACAGTCTGTGGACACCGGGGAGCTGTTTTTT |
| 37 | 12-TL647-TIL-CD8+_CD103+ | TIL | CD8+_CD103+ | TGTGCCAGCGGAGGCAGGGGGAACTACGAGCAGTACTTC |
| 38 | 12-TL647-TIL-CD8+_CD103+ | TIL | CD8+_CD103+ | TGTGCCAGCAGCGCCCCGGACAAAGCTAACTATGGCTACACCTTC |
| 39 | 12-TL647-TIL-CD8+_CD103+ | TIL | CD8+_CD103+ | TGTGCCAGCAGCGTAGATCCCGGGGTCTATGGCTACACCTTC |
| 40 | 12-TL647-TIL-CD8+_CD103+ | TIL | CD8+_CD103+ | TGCGCCAGCAGCTTGGAGGTGACAGCCACCTCAGATACGCAGTATTTT |
| 41 | 12-TL647-TIL-CD8+_CD103+ | TIL | CD8+_CD103+ | TGTGCCAGCAGCCCTCTAGCGGGAGATGGCGAGCAGTACTTC |
| 42 | 12-TL647-TIL-CD8+_CD103+ | TIL | CD8+_CD103+ | TGTGCCAGCAGCGTACTTAAGACAACCTGGAACACTGAAGCTTTCTTT |
| 43 | 12-TL647-TIL-CD8+_CD103+ | TIL | CD8+_CD103+ | TGTGCCAGCAGCCAAGTCCCAAGACCGGACTACGAGCAGTACTTC |
| 44 | 12-TL647-TIL-CD8+_CD103+ | TIL | CD8+_CD103+ | TGTGCCAGCAGTTCGCCGGGACAGGAAAAAAAGACCCAGTACTTC |
| 45 | 12-TL647-TIL-CD8+_CD103+ | TIL | CD8+_CD103+ | TGTGCCAGCAGTGAAGCAAGGGACGGAAGCTCCTACGAGCAGTACTTC |
| 46 | 12-TL647-TIL-CD8+_CD103+ | TIL | CD8+_CD103+ | TGTGCCAGCAGCGTAGGAAATAGAGGGGGCACAGATACGCAGTATTTT |
| 47 | 12-TL647-TIL-CD8+_CD103+ | TIL | CD8+_CD103+ | TGCAGCGTTGCGGTAGCGGGAGTGGGAGAGACCCAGTACTTC |
| 48 | 12-TL647-TIL-CD8+_CD103+ | TIL | CD8+_CD103+ | TGTGCCACCAGCAACGGGTTATCCTACGAGCAGTACTTC |
| 49 | 12-TL647-TIL-CD8+_CD103+ | TIL | CD8+_CD103+ | TGCGCCAGCAGTGGGGACGGTATGAACACTGAAGCTTTCTTT |
| 50 | 12-TL647-TIL-CD8+_CD103+ | TIL | CD8+_CD103+ | TGTGCCAGCAGCCAAGAAAACACTCACTACGAGCAGTACTTC |
| 51 | 12-TL647-TIL-CD8+_CD103+ | TIL | CD8+_CD103+ | TGTGCCAGCAGCTTAGTGGAGGCTCCCACTGAAGCTTTCTTT |
| 52 | 12-TL647-TIL-CD8+_CD103+ | TIL | CD8+_CD103+ | TGTGCCAGCAGTTCGCCGGGACAGGGAAAAAAGACCCAGTACTTC |
| 53 | 12-TL647-TIL-CD8+_CD103+ | TIL | CD8+_CD103+ | TGTGCCAGCAGTCATCTGGACGGAGGTCTCAATCAGCCCCAGCATTTT |
| 54 | 12-TL647-TIL-CD8+_CD103+ | TIL | CD8+_CD103+ | TGTGCCATCAGTGAGTCAGGGCCAGGGAACATTCAGTACTTC |
| 55 | 12-TL647-TIL-CD8+_CD103+ | TIL | CD8+_CD103+ | TGCAGCGTTGTTCGTAGGTTCGGGGAGCTGTTTTTT |
| 56 | 12-TL647-TIL-CD8+_CD103+ | TIL | CD8+_CD103+ | TGTGCCAGCAGTTACTCGGGCGGGTGGGGGGAGACCCAGTACTTC |
| 57 | 12-TL647-TIL-CD8+_CD103+ | TIL | CD8+_CD103+ | TGTGCCAGCAGGGGCGGGTATGAAACAGATACGCAGTATTTT |
| 58 | 12-TL647-TIL-CD8+_CD103+ | TIL | CD8+_CD103+ | TGTGCCATCAGTGCAACTATGGCTGGCTCCTACAATGAGCAGTTCTTC |
| 59 | 12-TL647-TIL-CD8+_CD103+ | TIL | CD8+_CD103+ | TGTGCCAGCAGTCCCTCATCCCTTGGACAGGACAATCAGCCCCAGCATTTT |
| 60 | 12-TL647-TIL-CD8+_CD103+ | TIL | CD8+_CD103+ | TGTGCCATTAACAGGGGGATGAACACTGAAGCTTTCTTT |
| 61 | 12-TL647-TIL-CD8+_CD103+ | TIL | CD8+_CD103+ | TGTGCCAGCAGCTTAGGGTCAGGGGAGTACATTCAGTACTTC |
| 62 | 12-TL647-TIL-CD8+_CD103+ | TIL | CD8+_CD103+ | TGCGCCAGCAGCCAAGATGCGACAGGGATCTACGAGCAGTACTTC |
| 63 | 12-TL647-TIL-CD8+_CD103+ | TIL | CD8+_CD103+ | TGTGCCAGCAGTGATGCTAGCGGGACCACAGATACGCAGTATTTT |
| 64 | 12-TL647-TIL-CD8+_CD103+ | TIL | CD8+_CD103+ | TGTGCCAGCAGTCCTCCGGGACTAGGTACAGATACGCAGTATTTT |
| 65 | 12-TL647-TIL-CD8+_CD103+ | TIL | CD8+_CD103+ | TGTGCCACCAGTGACAGCGGGAGACTGAACACCGGGGAGCTGTTTTTT |
| 66 | 12-TL647-TIL-CD8+_CD103+ | TIL | CD8+_CD103+ | TGCAGTGCTAGAAACCGGGACAGGGGCGCACATGGCTACACCTTC |
| 67 | 12-TL647-TIL-CD8+_CD103+ | TIL | CD8+_CD103+ | TGTGCCAGCAGCCAAGAAGGCAGGGAGGGGGAGACCCAGTACTTC |
| 68 | 12-TL647-TIL-CD8+_CD103+ | TIL | CD8+_CD103+ | TGTGCCAGCAGTCTCCTGGAGGCGGGTCAGCCCCAGCATTTT |
| 69 | 12-TL647-TIL-CD8+_CD103+ | TIL | CD8+_CD103+ | TGTGCCAGCAGTTTACTAGCGGGAGGGTTATACAATGAGCAGTTCTTC |
| 70 | 12-TL647-TIL-CD8+_CD103+ | TIL | CD8+_CD103+ | TGTGCCAGCAGTTACCGGACTAGCGGACTCAATGAGCAGTTCTTC |
| 71 | 12-TL647-TIL-CD8+_CD103+ | TIL | CD8+_CD103+ | TGTGCCAGCAGCGTTTCGACACGAACTGGGGCCAACGTCCTGACTTTC |
| 72 | 12-TL647-TIL-CD8+_CD103+ | TIL | CD8+_CD103+ | TGTGCCAGCAGCGCCGGCGGACACCACTCCTACGAGCAGTACTTC |
| 73 | 12-TL647-TIL-CD8+_CD103+ | TIL | CD8+_CD103+ | TGTGCCAGCAGCCCAACGGGGGGTCGGGACGAGCAGTACTTC |
| 74 | 12-TL647-TIL-CD8+_CD103+ | TIL | CD8+_CD103+ | TGCGCCAGCAGCCAAGCTAGCGGGGGGTCCACAGATACGCAGTATTTT |
| 75 | 12-TL647-TIL-CD8+_CD103+ | TIL | CD8+_CD103+ | TGTGCCAGCAGCTTAGCAGGAATAGACAACTATGGCTACACCTTC |
| 76 | 12-TL647-TIL-CD8+_CD103+ | TIL | CD8+_CD103+ | TGTGCCAGCAGCGTGGGGAGTAACTACAATGAGCAGTTCTTC |
| 77 | 12-TL647-TIL-CD8+_CD103+ | TIL | CD8+_CD103+ | TGTGCCAGCAGCTATCCAGAAGCTTTCTTT |
| 78 | 12-TL647-TIL-CD8+_CD103+ | TIL | CD8+_CD103+ | TGTGCCAGCAGTTACAGGGACAGGGGGGCAGATACGCAGTATTTT |
| 79 | 12-TL647-TIL-CD8+_CD103+ | TIL | CD8+_CD103+ | TGTGCCAGCAGAACTTATGGGGACATGAACACTGAAGCTTTCTTT |
| 80 | 12-TL647-TIL-CD8+_CD103+ | TIL | CD8+_CD103+ | TGTGCCAGCAGCCCGGGACAGGGTGGTAATTCACCCCTCCACTTT |
| 81 | 12-TL647-TIL-CD8+_CD103+ | TIL | CD8+_CD103+ | TGTGCCAGCAGCTTAAGGAGGGGGGGCACAGATACGCAGTATTTT |
| 82 | 12-TL647-TIL-CD8+_CD103+ | TIL | CD8+_CD103+ | TGTGCCAGCAGCTTATTTCCCGGGGAGCTGTTTTTT |
| 83 | 12-TL647-TIL-CD8+_CD103+ | TIL | CD8+_CD103+ | TGTGCCAGCAGCTTAGAGTCGTACAATGAGCAGTTCTTC |
| 84 | 12-TL647-TIL-CD8+_CD103+ | TIL | CD8+_CD103+ | TGTGCCAGCAGTTCCGGACAGAACACCGGGGAGCTGTTTTTT |
| 85 | 12-TL647-TIL-CD8+_CD103+ | TIL | CD8+_CD103+ | TGCAGTGCTAGAGAGGAGGACAGGGTGGACGAGCAGTACTTC |
| 86 | 12-TL647-TIL-CD8+_CD103+ | TIL | CD8+_CD103+ | TGCAGCGAGTGGACAGTGAACGGGGAGCTGTTTTTT |
| 87 | 12-TL647-TIL-CD8+_CD103+ | TIL | CD8+_CD103+ | TGTGCCATCAGTGGGTGGTGGACAGACTATGGCTACACCTTC |
| 88 | 12-TL647-TIL-CD8+_CD103+ | TIL | CD8+_CD103+ | TGTGCTAGCAGCTTGTGGGGGAGGCCTTCCGATGAGCAGTTCTTC |
| 89 | 12-TL647-TIL-CD8+_CD103+ | TIL | CD8+_CD103+ | TGTGCCAGCAGTTTATCAAATTCGGGCACTGAAGCTTTCTTT |
| 90 | 12-TL647-TIL-CD8+_CD103+ | TIL | CD8+_CD103+ | TGTGCCAGCAGCTGGGCCCGGACTGACGAGCAGTACTTC |
| 91 | 12-TL647-TIL-CD8+_CD103+ | TIL | CD8+_CD103+ | TGTGCCAGCAGCCAATCTACCCAGGGGTATTCACCCCTCCACTTT |
| 92 | 12-TL647-TIL-CD8+_CD103+ | TIL | CD8+_CD103+ | TGTGCCAGCAGCTCGGGGGGGGGCACTGAAGCTTTCTTT |
| 93 | 12-TL647-TIL-CD8+_CD103+ | TIL | CD8+_CD103+ | TGTGCCATCAGTGACGGGGGGTACACTGAAGCTTTCTTT |
| 94 | 12-TL647-TIL-CD8+_CD103+ | TIL | CD8+_CD103+ | TGTGCCAGCAGCGATCCCCTAGGCCCCTACTCTGGGGCC |
| | | | | AACGTCCTGACTTTC |
| 95 | 12-TL647-TIL-CD8+_CD103+ | TIL | CD8+_CD103+ | TGCAGCGTCGCCCCGAATAACTATGGCTACACCTTC |
| 96 | 12-TL647-TIL-CD8+_CD103+ | TIL | CD8+_CD103+ | TGTGCCAGCAGCCCCCTAGCGGGAGGGCCAGGCGAGCAGTACTTC |
| 97 | 12-TL647-TIL-CD8+_CD103+ | TIL | CD8+_CD103+ | TGTGCCAGCAGCCCCGGACAGGGAGGAAATTCACCCCTCCACTTT |
| 98 | 12-TL647-TIL-CD8+_CD103+ | TIL | CD8+_CD103+ | TGCAGTGCTGCCGGGACCACAAAAGAGGACGAGCAGTACTTC |
| 99 | 12-TL647-TIL-CD8+_CD103+ | TIL | CD8+_CD103+ | TGTGCCAGCAGCGCCGGCGGCGCGGGGGTGGAGGAAAAACTGTTTTTT |
| 100 | 13-TL647-TIL-CD8+_CD103− | TIL | CD8+_CD103− | TGTGCCATCAGTGAGTCAGGGCCAGGGAACATTCAGTACTTC |
| 101 | 13-TL647-TIL-CD8+_CD103− | TIL | CD8+_CD103− | TGTGCCAGCAGTAGACTAGCCCAAGAGACCCAGTACTTC |
| 102 | 13-TL647-TIL-CD8+_CD103− | TIL | CD8+_CD103− | TGTGCCAGCAGCCAAGAGAGCGGCGGCCCTTACAATGAGCAGTTCTTC |
| 103 | 13-TL647-TIL-CD8+_CD103− | TIL | CD8+_CD103− | TGCAGTGCTAGCGGCCTAGCGGGAGACGACGAGCAGTACTTC |
| 104 | 13-TL647-TIL-CD8+_CD103− | TIL | CD8+_CD103− | TGTGCCACCAGCAGAGTCGTGGGGAGTCACTATGGCTACACCTTC |
| 105 | 13-TL647-TIL-CD8+_CD103− | TIL | CD8+_CD103− | TGTGCCAGCAGTTTATGGCAGGGGTCTAATGAAAAACTGTTTTTT |
| 106 | 13-TL647-TIL-CD8+_CD103− | TIL | CD8+_CD103− | TGTGCCAGCAGCTTATCGGAGGGTAATGAGCAGTTCTTC |
| 107 | 13-TL647-TIL-CD8+_CD103− | TIL | CD8+_CD103− | TGTGCCAGCAGTTTCAACAATGAGCAGTTCTTC |
| 108 | 13-TL647-TIL-CD8+_CD103− | TIL | CD8+_CD103− | TGTGCCAGTTCAGGGGGCCGGACAGATACGCAGTATTTT |
| 109 | 13-TL647-TIL-CD8+_CD103− | TIL | CD8+_CD103− | TGCAGTGCTACCCCGGCTTACTTGAACACCGGGGAGCTGTTTTTT |
| 110 | 13-TL647-TIL-CD8+_CD103− | TIL | CD8+_CD103− | TGTGCCAGCAGTTACCAAACCGGGACAGGGGTCTATGGCTACACCTTC |
| 111 | 13-TL647-TIL-CD8+_CD103− | TIL | CD8+_CD103− | TGTGCCAGCGGTCAAGGGGGGGCTTGGGGCTACACCTTC |
| 112 | 13-TL647-TIL-CD8+_CD103− | TIL | CD8+_CD103− | TGTGCCAGCAGCCGGGGCAGCTCTCGACCMGAACACTGAAGCTTTCTTT |
| 113 | 13-TL647-TIL-CD8+_CD103− | TIL | CD8+_CD103− | TGTGCCAGCAGCCCAACGGGGGGTCGGGACGAGCAGTACTTC |
| 114 | 13-TL647-TIL-CD8+_CD103− | TIL | CD8+_CD103− | TGTGCCAGCTTACGGGCAGGGAGCCCCTCAATGGAGACCCAGTACTTC |
| 115 | 13-TL647-TIL-CD8+_CD103− | TIL | CD8+_CD103− | TGCAGCGTTTCCGGGACAGGGGTATACAATGAGCAGTTCTTC |
| 116 | 13-TL647-TIL-CD8+_CD103− | TIL | CD8+_CD103− | TGTGCCAGCAGTCAAGGGGGCGCCCTAGGCTACACCTTC |
| 117 | 13-TL647-TIL-CD8+_CD103− | TIL | CD8+_CD103− | TGTGCCAGCACCAACCTGGAGGGGACGGGGAGACTAGCCAAAAACATT |
| | | | | CAGTACTTC |
| 118 | 13-TL647-TIL-CD8+_CD103− | TIL | CD8+_CD103− | TGTGCCAGCGGCGACGGAGGCACAGATACGCAGTATTTT |
| 119 | 13-TL647-TIL-CD8+_CD103− | TIL | CD8+_CD103− | TGTGCCAGCAGTCCCCGGGACAGGGGGCGGGAGCAGTACTTC |
| 120 | 13-TL647-TIL-CD8+_CD103− | TIL | CD8+_CD103− | TGCGCCAGTAGGACAGGCAATGAGCAGTTCTTC |
| 121 | 13-TL647-TIL-CD8+_CD103− | TIL | CD8+_CD103− | TGTGCCAGCAGCTTGCACCTAAGCGGGGACTACAATGAGCAGTTCTTC |
| 122 | 13-TL647-TIL-CD8+_CD103− | TIL | CD8+_CD103− | TGCGCCAGCAGCTGCAGGGGCCTGACCCTGAACACTGAAGCTTTCTTT |
| 123 | 13-TL647-TIL-CD8+_CD103− | TIL | CD8+_CD103− | TGTGCCAGCAGCTTAGGGCCAGCCAGGGTGGGGGAAGAGACCCAGTACTTC |
| 124 | 13-TL647-TIL-CD8+_CD103− | TIL | CD8+_CD103− | TGTGCCAGCAGCCCTGGGGGCTACAATGAGCAGTTCTTC |
| 125 | 13-TL647-TIL-CD8+_CD103− | TIL | CD8+_CD103− | TGTGCCAGCAGCTTAGGAGTCCAAGAGACCCAGTACTTC |
| 126 | 13-TL647-TIL-CD8+_CD103− | TIL | CD8+_CD103− | TGTGCCAGCAGCTTACGGCAAAGCAGGCAGAACACTGAAGCTTTCTTT |
| 127 | 13-TL647-TIL-CD8+_CD103− | TIL | CD8+_CD103− | TGTGCCAGCAGTTTAGGAGGACAGCCCTATGGCTACACCTTC |
| 128 | 13-TL647-TIL-CD8+_CD103− | TIL | CD8+_CD103− | TGTGCCAGCAGTCCCCTACGTGCGGGCGGCGGACCAGATACGCAGTATTTT |
| 129 | 13-TL647-TIL-CD8+_CD103− | TIL | CD8+_CD103− | TGTGCCAGCAGCGTAGGGGTAGCCGTGGTGGACGAGCAGTACTTC |
| 130 | 13-TL647-TIL-CD8+_CD103− | TIL | CD8+_CD103− | TGTGCCAGCAGCTTGTGGGGATTTGGGGGGACCTACGAGCAGTACTTC |
| 131 | 13-TL647-TIL-CD8+_CD103− | TIL | CD8+_CD103− | TGTGCCAGTAGTATAGGGCAGCAAGAGCAGTTCTTC |
| 132 | 13-TL647-TIL-CD8+_CD103− | TIL | CD8+_CD103− | TGTGCCAGCAGCCAGGGGGGGGCTCTTGGCTACACCTTC |
| 133 | 13-TL647-TIL-CD8+_CD103− | TIL | CD8+_CD103− | TGCGCCAGCAGCTCCGGACAGGGGGCCACTGAAGCTTTCTTT |
| 134 | 13-TL647-TIL-CD8+_CD103− | TIL | CD8+_CD103− | TGTGCCAGCAGTGAAGTTGGTGTTTACGAGCAGTACTTC |
| 135 | 13-TL647-TIL-CD8+_CD103− | TIL | CD8+_CD103− | TGCGCCAGTTGCCCAACTACGTGCACTGAAGCTTTCTTT |
| 136 | 13-TL647-TIL-CD8+_CD103− | TIL | CD8+_CD103− | TGCAGTGCTAGAGACCAGGACAGGTTAAACTATGGCTACACCTTC |
| 137 | 13-TL647-TIL-CD8+_CD103− | TIL | CD8+_CD103− | TGTGCCAGCAGCTGGGGGGGCGATTCACCCCTCCACTTT |
| 138 | 13-TL647-TIL-CD8+_CD103− | TIL | CD8+_CD103− | TGCGCCAGCAGCTTCCTGGACAGCTTGAACACTGAAGCTTTCTTT |
| 139 | 13-TL647-TIL-CD8+_CD103− | TIL | CD8+_CD103− | TGCGCCAGCAGCCAAGATCCAACGGACTCCTACGAGCAGTACTTC |
| 140 | 13-TL647-TIL-CD8+_CD103− | TIL | CD8+_CD103− | TGCAGCGTTGAATTTGGTTACGAGCAGTACTTC |
| 141 | 13-TL647-TIL-CD8+_CD103− | TIL | CD8+_CD103− | TGTGCCACCAGCACTTATAACACCGGGGAGCTGTTTTTT |
| 142 | 13-TL647-TIL-CD8+_CD103− | TIL | CD8+_CD103− | TGCAGCGGGGAGGGAGGACAGCTAGACGGCTACACCTTC |
| 143 | 13-TL647-TIL-CD8+_CD103− | TIL | CD8+_CD103− | TGTGCCAGCAGTCCACCTAGGGGTGAGCAGTTCTTC |
| 144 | 13-TL647-TIL-CD8+_CD103− | TIL | CD8+_CD103− | TGCAGCGTCGAGCCTCCCAACACCGGGGAGCTGTTTTTT |
| 145 | 13-TL647-TIL-CD8+_CD103− | TIL | CD8+_CD103− | TGTGCCAGCAGCTTGGGCACAAATGAGCAGTTCTTC |
| 146 | 13-TL647-TIL-CD8+_CD103− | TIL | CD8+_CD103− | TGTGCCAGCAGCCAAGTCGCTAGCGGGGGGCGCGAGCAGTACTTC |
| 147 | 13-TL647-TIL-CD8+_CD103− | TIL | CD8+_CD103− | TGTGCCAGCAGCTTAGATTCCCCGTTGAACACTGAAGCTTTCTTT |
| 148 | 13-TL647-TIL-CD8+_CD103− | TIL | CD8+_CD103− | TGTGCCAGCAGCTTAGGAGGGGGCTTATACGAGCAGTACTTC |
| 149 | 13-TL647-TIL-CD8+_CD103− | TIL | CD8+_CD103− | TGTGCCAGCAGTGAACGGAGGATAGCAGGTCAAGAGACCCAGTACTTC |
| 150 | 13-TL647-TIL-CD8+_CD103− | TIL | CD8+_CD103− | TGTGCCAGCAGCTTAGCGTCAGGGGGCTCGGGCACTGAAGCTTTCTTT |
| 151 | 13-TL647-TIL-CD8+_CD103− | TIL | CD8+_CD103− | TGTGCCAGCAGCCTTCAATGGACAGGGGTGCAGGAGCAGTTCTTC |
| 152 | 13-TL647-TIL-CD8+_CD103− | TIL | CD8+_CD103− | TGTGCCAGCAGTGAATACCGGGTTGGGGTCACTGAAGCTTTCTTT |
| 153 | 13-TL647-TIL-CD8+_CD103− | TIL | CD8+_CD103− | TGCAGTGCCTTCACAGGGTACACCGGGGAGCTGTTTTTT |
| 154 | 13-TL647-TIL-CD8+_CD103− | TIL | CD8+_CD103− | TGTGCCAGCAGCCCCCTGGGCGCGGGAGTAGGTGAGCAGTTCTTC |
| 155 | 13-TL647-TIL-CD8+_CD103− | TIL | CD8+_CD103− | TGTGCCAGCAGCTTAGAGGACAGGGGACGTGAGCAGTTCTTC |
| 156 | 13-TL647-TIL-CD8+_CD103− | TIL | CD8+_CD103− | TGCGCCAGCAGTGAGTTAGACCGGGGACGGGACTATGGCTACACCTTC |
| 157 | 13-TL647-TIL-CD8+_CD103− | TIL | CD8+_CD103− | TGTGCCAGCAGTGAAGCGCTTCTAGCGGAGACCACAGATACGCAGTATTTT |
| 158 | 13-TL647-TIL-CD8+_CD103− | TIL | CD8+_CD103− | TGTGCCAGCAGCTTAGGGTCAGGGGAGTACATTCAGTACTTC |
| 159 | 13-TL647-TIL-CD8+_CD103− | TIL | CD8+_CD103− | TGTGCCAGCAGCTTAGGAACTAGCGGACCCTACGAGCAGTACTTC |
| 160 | 13-TL647-TIL-CD8+_CD103− | TIL | CD8+_CD103− | TGTGCCAGCAGTCCGCCCGCTTCAGGGGGCACTGAAGATACGCAGTATTTT |
| 161 | 13-TL647-TIL-CD8+_CD103− | TIL | CD8+_CD103− | TGTGCCAGCAGCCCCCATGAGCGGTTAGGGAATGAGCAGTTCTTC |
| 162 | 13-TL647-TIL-CD8+_CD103− | TIL | CD8+_CD103− | TGCAGTGCTGCCGGGACCACAAAAGAGGACGAGCAGTACTTC |
| 163 | 13-TL647-TIL-CD8+_CD103− | TIL | CD8+_CD103− | TGTGCCAGAACCCTGGGGACCGGGGGCCGCTCCTACAATGAGCAGTTCTTC |
| 164 | 13-TL647-TIL-CD8+_CD103− | TIL | CD8+_CD103− | TGCGCCAGCAGCTTGGAGGGACAGGGCCCATATGGCTACACCTTC |
| 165 | 13-TL647-TIL-CD8+_CD103− | TIL | CD8+_CD103− | TGTGCCAGCAGAAATACAGGGGCCTACCGTTCCTACAATGAGCAGTTCTTC |
| 166 | 13-TL647-TIL-CD8+_CD103− | TIL | CD8+_CD103− | TGTGCCAGCAGCGTAAATAGCGGTGAGCAGTTCTTC |
| 167 | 139-TL706-TIL-CD8+_CD103+ | TIL | CD8+_CD103+ | TGTGCCAGCAGCCAAGATGGGACAGGGGCCTACGAGCAGTACTTC |
| 168 | 139-TL706-TIL-CD8+_CD103+ | TIL | CD8+_CD103+ | TGTGCCAGCAGTTTGAGCAGGACCTACGAGCAGTACTTC |
| 169 | 139-TL706-TIL-CD8+_CD103+ | TIL | CD8+_CD103+ | TGCGCCAGCAGCTCGAGGTGGGACTTCCAAGAGACCCAGTACTTC |
| 170 | 139-TL706-TIL-CD8+_CD103+ | TIL | CD8+_CD103+ | TGTGCCAGCAGTGACCGGGACAGGGCCACAGATACGCAGTATTTT |
| 171 | 139-TL706-TIL-CD8+_CD103+ | TIL | CD8+_CD103+ | TGCAGTGCTATTCAGGGTTTGGGCACAGATACGCAGTATTTT |
| 172 | 139-TL706-TIL-CD8+_CD103+ | TIL | CD8+_CD103+ | TGTGCCAGCAGCTGGGGGACGACCTACGAGCAGTACTTC |
| 173 | 139-TL706-TIL-CD8+_CD103+ | TIL | CD8+_CD103+ | TGTGCCAGCAGTTTCAGGGGGTACGAGCAGTACTTC |
| 174 | 139-TL706-TIL-CD8+_CD103+ | TIL | CD8+_CD103+ | TGTGCCAGCAGCTTAGGGGCGGGGGGTAATGAGCAGTTCTTC |
| 175 | 139-TL706-TIL-CD8+_CD103+ | TIL | CD8+_CD103+ | TGTGCCAGCAGTTACACCCCCTACGGGGGGGCCGCGACCCAGTACTTC |
| 176 | 139-TL706-TIL-CD8+_CD103+ | TIL | CD8+_CD103+ | TGTGCCAGGACCCACGACTACCTGGATAGCGGGGGGGCCGCAGATACG |
| | | | | CAGTATTTT |
| 177 | 139-TL706-TIL-CD8+_CD103+ | TIL | CD8+_CD103+ | TGTGCCAGCAGCTTAGCCGGGACTAGCGGGGGGGCCGTCGGGGAGCTG |
| | | | | TTTTTT |
| 178 | 139-TL706-TIL-CD8+_CD103+ | TIL | CD8+_CD103+ | TGTGCCAGCAGCCACCCCGGACTAGCGGGGGGCCGGTACCAAGAGACC |
| | | | | CAGTACTTC |
| 179 | 139-TL706-TIL-CD8+_CD103+ | TIL | CD8+_CD103+ | TGTGCCAGCAGCGTAGGACTAGGCAGCCAAGAGACCCAGTACTTC |
| 180 | 139-TL706-TIL-CD8+_CD103+ | TIL | CD8+_CD103+ | TGTGCCAGCAGCCCTGAGACTGAAGCTTTCTTT |
| 181 | 139-TL706-TIL-CD8+_CD103+ | TIL | CD8+_CD103+ | TGCGCCAGCAGACCAAGACTCAAAGGCGACGGGACAGATACGCAGTATTTT |
| 182 | 139-TL706-TIL-CD8+_CD103+ | TIL | CD8+_CD103+ | TGTGCTAGTCGCCTTATAACCAAGGCGAACACCGGGGAGCTGTTTTTT |
| 183 | 139-TL706-TIL-CD8+_CD103+ | TIL | CD8+_CD103+ | TGTGCCATCAGTGAGAATGAGGGTAATGAGCAGTTCTTC |
| 184 | 139-TL706-TIL-CD8+_CD103+ | TIL | CD8+_CD103+ | TGTGCCAGCAGTTACGGACAGGGAGCCTACGAGCAGTACTTC |
| 185 | 139-TL706-TIL-CD8+_CD103+ | TIL | CD8+_CD103+ | TGTGCCAGCAGCCCTCGACTAGCGGGGGGCCGTCAAAGCACA |
| | | | | GATACGCAGTATTTT |
| 186 | 139-TL706-TIL-CD8+_CD103+ | TIL | CD8+_CD103+ | TGTGCCAGCAGTTTATTGCAGGGCACAGATACGCAGTATTTT |
| 187 | 139-TL706-TIL-CD8+_CD103+ | TIL | CD8+_CD103+ | TGTGCCACCAGTGGGCGGGTGGCAGCCCGATACTACAATGAGCAGTTCTTC |
| 188 | 139-TL706-TIL-CD8+_CD103+ | TIL | CD8+_CD103+ | TGTGCCATCAGTGAGACCCGACAGGGAGGCGTGAGGACTGAAGCTTTCTTT |
| 189 | 139-TL706-TIL-CD8+_CD103+ | TIL | CD8+_CD103+ | TGTGCCAGCAGCCGAGTTAGAGGGGGGTACAATGAGCAGTTCTTC |
| 190 | 139-TL706-TIL-CD8+_CD103+ | TIL | CD8+_CD103+ | TGCGCCAGCAGCTTTGGAGGGCAGGAGTACTCTGGAAACACCATATATTTT |
| 191 | 139-TL706-TIL-CD8+_CD103+ | TIL | CD8+_CD103+ | TGTGCCAGCAGTGACCTAAGGTGGGGACAGTACCAAGAGACCCAGTACTTC |
| 192 | 139-TL706-TIL-CD8+_CD103+ | TIL | CD8+_CD103+ | TGTGCCACCACGTTGGACCTTACTAGCGGGGGGGAGGATACGCAGTATTTT |
| 193 | 139-TL706-TIL-CD8+_CD103+ | TIL | CD8+_CD103+ | TGTGCCAGCAGCCAAGATGGGACGAACACCGGGGAGCTGTTTTTT |
| 194 | 139-TL706-TIL-CD8+_CD103+ | TIL | CD8+_CD103+ | TGCGCCAGCAGCCCCTGGGGTAGCGGGGGGCAGGAGACCCAGTACTTC |
| 195 | 139-TL706-TIL-CD8+_CD103+ | TIL | CD8+_CD103+ | TGTGCCAGCACCGAGGCGACAGGAACCTCCTACAATGAGCAGTTCTTC |
| 196 | 139-TL706-TIL-CD8+_CD103+ | TIL | CD8+_CD103+ | TGTGCCAGCAGCTTAGGAAATGGGGCTTATAATTCACCCCTCCACTTT |
| 197 | 139-TL706-TIL-CD8+_CD103+ | TIL | CD8+_CD103+ | TGTGCCATCAGTGACACAGGAGCCCGCCACTATGGCTACACCTTC |
| 198 | 139-TL706-TIL-CD8+_CD103+ | TIL | CD8+_CD103+ | TGTGCCACCAGACCAGCATTACAAGAGACCCAGTACTTC |
| 199 | 139-TL706-TIL-CD8+_CD103+ | TIL | CD8+_CD103+ | TGCGCCAGCAGCTTGTTCGGCTACAATGAGCAGTTCTTC |
| 200 | 139-TL706-TIL-CD8+_CD103+ | TIL | CD8+_CD103+ | TGTGCCAGCAGCTGGGGACTAGCGGAGACCAATGAGCAGTTCTTC |
| 201 | 139-TL706-TIL-CD8+_CD103+ | TIL | CD8+_CD103+ | TGTGCCAGCAGCTTACCGGGACAGATGAACACTGAAGCTTTCTTT |
| 202 | 139-TL706-TIL-CD8+_CD103+ | TIL | CD8+_CD103+ | TGTGCCAGCAGTTTACGAGGAAGGGTACCCAAAAACATTCAGTACTTC |
| 203 | 139-TL706-TIL-CD8+_CD103+ | TIL | CD8+_CD103+ | TGTGCCAGCAGCTCGGAGGGGGCGCTTGGAAACACCATATATTTT |
| 204 | 139-TL706-TIL-CD8+_CD103+ | TIL | CD8+_CD103+ | TGTGCCAGCAGCCAGGGGACAGGGACTATATACGAGCAGTACTTC |
| 205 | 139-TL706-TIL-CD8+_CD103+ | TIL | CD8+_CD103+ | TGTGCCAGCAGCTTAGCGGCAGGGGACCCTTCTGGGGCCAACGTC |
| | | | | CTGACTTTC |
| 206 | 139-TL706-TIL-CD8+_CD103+ | TIL | CD8+_CD103+ | TGTGCCAGCAGTTACTCGGCTTTCGCGGCGAGCTATGGCTACACCTTC |
| 207 | 139-TL706-TIL-CD8+_CD103+ | TIL | CD8+_CD103+ | TGTGCCAGCAGTTCGTACAGGACAAATGAAAAACTGTTTTTT |
| 208 | 139-TL706-TIL-CD8+_CD103+ | TIL | CD8+_CD103+ | TGTGCCAGCAGCGCCGGGGAGCTGTTTTTT |
| 209 | 139-TL706-TIL-CD8+_CD103+ | TIL | CD8+_CD103+ | TGTGCCAGCAGTCGCGCCTCGCCTGACAGGGGGTTTTTGTAC |
| | | | | ACCGGGGAGCTGTTTTTT |
| 210 | 139-TL706-TIL-CD8+_CD103+ | TIL | CD8+_CD103+ | TGTGCCAGCAGTGAGGAGGGTTGGATTCGTATCTTC |
| 211 | 139-TL706-TIL-CD8+_CD103+ | TIL | CD8+_CD103+ | TGTGCCACTCGGGGACAGAGCCTCCAAGAGACCCAGTACTTC |
| 212 | 139-TL706-TIL-CD8+_CD103+ | TIL | CD8+_CD103+ | TGTGCCAGCAGCTCTCCCCCGGGACTAGCGGGGGGGCCTGGG |
| | | | | GATACGCAGTATTTT |
| 213 | 139-TL706-TIL-CD8+_CD103+ | TIL | CD8+_CD103+ | TGTGCCAGCAGTTTATCTAAGGGAAGCAGGGCTAACTATGGCTACACCTTC |
| 214 | 139-TL706-TIL-CD8+_CD103+ | TIL | CD8+_CD103+ | TGCGCCAGCAGCCAAGGTGGCGAGCAGTACTTC |
| 215 | 139-TL706-TIL-CD8+_CD103+ | TIL | CD8+_CD103+ | TGTGCCAGCAGTTACTCGGACGTGGCCAAAAACATTCAGTACTTC |
| 216 | 139-TL706-TIL-CD8+_CD103+ | TIL | CD8+_CD103+ | TGTGCCAGTAGTATACGCTCGGCTTACGAGCAGTACTTC |
| 217 | 139-TL706-TIL-CD8+_CD103+ | TIL | CD8+_CD103+ | TGTGCCAGCAGTTACTCCCCCTATAACTCCTACGAGCAGTACTTC |
| 218 | 139-TL706-TIL-CD8+_CD103+ | TIL | CD8+_CD103+ | TGTGCCAGCAGCCCGACAGGGGCTCCCGGGGAGCTGTTTTTT |
| 219 | 139-TL706-TIL-CD8+_CD103+ | TIL | CD8+_CD103+ | TGTGCCAGCAGTGCTAGCGGGGGATTTCTCGGAGATACGCAGTATTTT |
| 220 | 139-TL706-TIL-CD8+_CD103+ | TIL | CD8+_CD103+ | TGTGCCAGCAGCCAAGTTCGCAGGGGGCGATCACCCCTCCACTTT |
| 221 | 139-TL706-TIL-CD8+_CD103+ | TIL | CD8+_CD103+ | TGCGCCAGCAGCCAATCACGGACTAGCGGGGGGCGGGGAGAGCAGTTCTTC |
| 222 | 139-TL706-TIL-CD8+_CD103+ | TIL | CD8+_CD103+ | TGTGCCAGCACCCACCGGGACCGTAACTACGAGCAGTACTTC |
| 223 | 139-TL706-TIL-CD8+_CD103+ | TIL | CD8+_CD103+ | TGCAGTGCTAGGGAGCATCAGGGACGAGAGAACACCGGGGAGCTGTTTTTT |
| 224 | 139-TL706-TIL-CD8+_CD103+ | TIL | CD8+_CD103+ | TGTGCCAGCAGCTTACGGGCCTACGAGCAGTACTTC |
| 225 | 139-TL706-TIL-CD8+_CD103+ | TIL | CD8+_CD103+ | TGTGCCAGCACGGGTCAGCTTTACACCGGGGAGCTGTTTTTT |
| 226 | 139-TL706-TIL-CD8+_CD103+ | TIL | CD8+_CD103+ | TGTGCCAGCAGTTGGAGGCCAGCTGGAAACACCATATATTTT |
| 227 | 139-TL706-TIL-CD8+_CD103+ | TIL | CD8+_CD103+ | TGTGCCAGCAGCGACAGGGGATTCTTCGATGAGCAGTTCTTC |
| 228 | 139-TL706-TIL-CD8+_CD103+ | TIL | CD8+_CD103+ | TGTGCCAGCAGTTCCCCGGACAGGGGCTACAATGAGCAGTTCTTC |
| 229 | 139-TL706-TIL-CD8+_CD103+ | TIL | CD8+_CD103+ | TGTGCCAGCAGCTTCCCCCAAGAGACCATGAACACTGAAGCTTTCTTT |
| 230 | 139-TL706-TIL-CD8+_CD103+ | TIL | CD8+_CD103+ | TGTGCCAGCGGCACTTACGGGGCAAACTACGAGCAGTACTTC |
| 231 | 139-TL706-TIL-CD8+_CD103+ | TIL | CD8+_CD103+ | TGTGCCACCAGCGTGGGACAGTTCTACGAGCAGTACTTC |
| 232 | 139-TL706-TIL-CD8+_CD103+ | TIL | CD8+_CD103+ | TGTGCCAGCCGGGGGGACAGAGGCATGAACACTGAAGCTTTCTTT |
| 233 | 139-TL706-TIL-CD8+_CD103+ | TIL | CD8+_CD103+ | TGTGCCAGCAGCTATGGGGCGGACAGACTCATCTACAATGAGCAGTTCTTC |
| 234 | 139-TL706-TIL-CD8+_CD103+ | TIL | CD8+_CD103+ | TGTGCCAGCAGCTCGCCGGACAGGGGTGCCACTGAAGCTTTCTTT |
| 235 | 139-TL706-TIL-CD8+_CD103+ | TIL | CD8+_CD103+ | TGTGCCAGCAGCCAAGATCGAAGGCCTCGATCTGGAAACACCATATATTTT |
| 236 | 139-TL706-TIL-CD8+_CD103+ | TIL | CD8+_CD103+ | TGTGCCAGCAGCAGAAATCAGCCCCAGCATTTT |
| 237 | 139-TL706-TIL-CD8+_CD103+ | TIL | CD8+_CD103+ | TGCAGCGTTGAAGACGGGACTAGCGATAGAGAGACCCAGTACTTC |
| 238 | 139-TL706-TIL-CD8+_CD103+ | TIL | CD8+_CD103+ | TGCGCCAGCAGTGAGTCCGGAGCAGGTTACGTTCCCTACAATGA |
| | | | | GCAGTTCTTC |
| 239 | 139-TL706-TIL-CD8+_CD103+ | TIL | CD8+_CD103+ | TGCGCCAGCAGCTCCCGGACTAGCGGGGGGGCAGGAGAGCAGTACTTC |
| 240 | 139-TL706-TIL-CD8+_CD103+ | TIL | CD8+_CD103+ | TGTGCCAGCAGCTTATCGCTCCTCGTAGGAGGGGGAGTCAAAAA |
| | | | | CATTCAGTACTTC |
| 241 | 139-TL706-TIL-CD8+_CD103+ | TIL | CD8+_CD103+ | TGTGCCAGCAGCTTAGATGGGCCTCTGGATACGCAGTATTTT |
| 242 | 139-TL706-TIL-CD8+_CD103+ | TIL | CD8+_CD103+ | TGTGCCAGCAGTTATACGGCCAACTACGAGCAGTACTTC |
| 243 | 139-TL706-TIL-CD8+_CD103+ | TIL | CD8+_CD103+ | TGTGCCAGCAGTTACCGGACTAGCGGGGGTTTAAACACC |
| | | | | GGGGAGCTGTTTTTT |
| 244 | 139-TL706-TIL-CD8+_CD103+ | TIL | CD8+_CD103+ | TGTGCCAGCAGCCCCCGCATTATCTGGTGGGTCTCTCTC |
| | | | | TCTGGGGCCAACGTCCTGACTTTC |
| 245 | 139-TL706-TIL-CD8+_CD103+ | TIL | CD8+_CD103+ | TGTGCCAGCAGTTACGGGCGGGGGGGCCACAATGAGCAGTTCTTC |
| 246 | 139-TL706-TIL-CD8+_CD103+ | TIL | CD8+_CD103+ | TGTGCCAGCAGCTTAGTTGGGCCGGGGGCGCGCGGCTACACCTTC |
| 247 | 139-TL706-TIL-CD8+_CD103+ | TIL | CD8+_CD103+ | TGCAGTGCTAGAGATCTTAACAGCGTCGCCCCAAGCGGCGAGCAGTACTTC |
| 248 | 139-TL706-TIL-CD8+_CD103+ | TIL | CD8+_CD103+ | TGTGCCAGCAGCTACCGGGACAGACTCAATGAGCAGTTCTTC |
| 249 | 139-TL706-TIL-CD8+_CD103+ | TIL | CD8+_CD103+ | TGTGCCAGCACGGGTCAGCTTTACACCGGGGAGCTGTTTTTT |
| 250 | 139-TL706-TIL-CD8+_CD103+ | TIL | CD8+_CD103+ | TGCAGTGCTAGAGATAGGGGGTGGTGGGACAATGAGCAGTTCTTC |
| 251 | 139-TL706-TIL-CD8+_CD103+ | TIL | CD8+_CD103+ | TGCAGCGTTGAGGACAGCGACCGGGGCGAGCAGTACTTC |
| 252 | 139-TL706-TIL-CD8+_CD103+ | TIL | CD8+_CD103+ | TGCAGTGCTAGAGATGATGGCTCCGCTTCTTATCTTAGCAAT |
| | | | | CAGCCCCAGCATTTT |
| 253 | 139-TL706-TIL-CD8+_CD103+ | TIL | CD8+_CD103+ | TGTGCCACGCAACCGACAGGGGGCTTCTACGAGCAGTACTTC |
| 254 | 139-TL706-TIL-CD8+_CD103+ | TIL | CD8+_CD103+ | TGTGCCAGCAGTTACTCTCTCGCCCGGAGCCAGTACTTC |
| 255 | 139-TL706-TIL-CD8+_CD103+ | TIL | CD8+_CD103+ | TGTGCCAGCAGCCCGTCGACAGGGCGCTCTGGAAACACCATATATTTT |
| 256 | 139-TL706-TIL-CD8+_CD103+ | TIL | CD8+_CD103+ | TGTGCCAGCAGCCCGACAGGGATTGAAGCTTTCTTT |
| 257 | 139-TL706-TIL-CD8+_CD103+ | TIL | CD8+_CD103+ | TGTGCCAGCAGCCCATTTATAGAGGCCTTTACGCAGTATTTT |
| 258 | 139-TL706-TIL-CD8+_CD103+ | TIL | CD8+_CD103+ | TGTGCCAGTAGCATGACAGGGGGCTGGGGTCAGCCCCAGCATTTT |
| 259 | 139-TL706-TIL-CD8+_CD103+ | TIL | CD8+_CD103+ | TGCAGTGTAGGGGGCCAAGGCTACGAGCAGTACTTC |
| 260 | 139-TL706-TIL-CD8+_CD103+ | TIL | CD8+_CD103+ | TGTGCCAGCAGTGACGACCCGGGGCTTGGCACAGATACGCAGTATTTT |
| 261 | 139-TL706-TIL-CD8+_CD103+ | TIL | CD8+_CD103+ | TGTGCCATCAGTGAAATCAATGAGCAGTTCTTC |
| 262 | 139-TL706-TIL-CD8+_CD103+ | TIL | CD8+_CD103+ | TGTGCCAGCAAGGGACAGGGGATTTATGGCTACACCTTC |
| 263 | 139-TL706-TIL-CD8+_CD103+ | TIL | CD8+_CD103+ | TGTGCCAGCAGTCCACATCTGGGACAGATACCTTACAATGAGCAGTTCTTC |
| 264 | 139-TL706-TIL-CD8+_CD103+ | TIL | CD8+_CD103+ | TGTGCCAGCAGTTACTCCCCTTGGCGGGGGCATGAGCAGTTCTTC |
| 265 | 139-TL706-TIL-CD8+_CD103+ | TIL | CD8+_CD103+ | TGTGCCAGCTCTTTGGTGGGCTCCTACGAGCAGTACTTC |
| 266 | 139-TL706-TIL-CD8+_CD103+ | TIL | CD8+_CD103+ | TGTGCCAGCTCGCCTCCCACGGCGGAGGGATACTACGAGCAGTACTTC |
| 267 | 139-TL706-TIL-CD8+_CD103+ | TIL | CD8+_CD103+ | TGCGCCAGCAGCCACCCGGGACACGAGTCCTACGAGCAGTACTTC |
| 268 | 139-TL706-TIL-CD8+_CD103+ | TIL | CD8+_CD103+ | TGCGCCAGCAGCCTAGGACAGACGGCGCAAAAACTGTTTTTT |
| 269 | 139-TL706-TIL-CD8+_CD103+ | TIL | CD8+_CD103+ | TGTGCCAGCAGCCCCAACGGCGATGAGCAGTTCTTC |
| 270 | 139-TL706-TIL-CD8+_CD103+ | TIL | CD8+_CD103+ | TGCAGCGTTGAATTGACGGAAGCTTTCTTT |
| 271 | 139-TL706-TIL-CD8+_CD103+ | TIL | CD8+_CD103+ | TGTGCCAGCAGTTACTCTAGGAGCAATCAGCCCCAGCATTTT |
| 272 | 139-TL706-TIL-CD8+_CD103+ | TIL | CD8+_CD103+ | TGTGCCACCAGCAGAGGGGCGACCTTCTACGAGCAGTACTTC |
| 273 | 139-TL706-TIL-CD8+_CD103+ | TIL | CD8+_CD103+ | TGTGCCAGCAGCTTAGCGGCAGGGGGCCCTTCTGGGGCCAAC |
| | | | | GTCCTGACTTTC |
| 274 | 139-TL706-TIL-CD8+_CD103+ | TIL | CD8+_CD103+ | TGTGCCATCAGTGAGTGGGCAGGTAGCAATCAGCCCCAGCATTTT |
| 275 | 139-TL706-TIL-CD8+_CD103+ | TIL | CD8+_CD103+ | TGTGCCAGCTCACCATTCCCCCCCCAGAGCTCCTACAATGAGCAGTTCTTC |
| 276 | 139-TL706-TIL-CD8+_CD103+ | TIL | CD8+_CD103+ | TGCAGTGCTAGGGGCCCTCAGGGGTACTACGAGCAGTACTTC |
| 277 | 139-TL706-TIL-CD8+_CD103+ | TIL | CD8+_CD103+ | TGTGCCAGCAGCGTAGGGGTCCCGACAGGGGGAGACTCACCCCTCCACTTT |
| 278 | 139-TL706-TIL-CD8+_CD103+ | TIL | CD8+_CD103+ | TGTGCCAGCAGTTACACGCCTTGGGCAGGACCCTACGAGCAGTACTTC |
| 279 | 139-TL706-TIL-CD8+_CD103+ | TIL | CD8+_CD103+ | TGTGCCAGCAGCCCCATCCTACGGGGGGACTACAATGAGCAGTTCTTC |
| 280 | 139-TL706-TIL-CD8+_CD103+ | TIL | CD8+_CD103+ | TGTGCCAGTAGTATCTTAGGACTCACCGGGGAGCTGTTTTTT |
| 281 | 139-TL706-TIL-CD8+_CD103+ | TIL | CD8+_CD103+ | TGTGCCAGCAGTGGACTAAATTCACCCCTCCACTTT |
| 282 | 139-TL706-TIL-CD8+_CD103+ | TIL | CD8+_CD103+ | TGTGCCAGCAGCGCGCGGGTCGCGGTAGCGGGGGGAC |
| | | | | TAAGCTCCTACAATGAGCAGTTCTTC |
| 283 | 139-TL706-TIL-CD8+_CD103+ | TIL | CD8+_CD103+ | TGCGCCAGCAGCTTGTTAGGCCCCGTCGGCAGGGGTGATGACGAG |
| | | | | CAGTACTTC |
| 284 | 139-TL706-TIL-CD8+_CD103+ | TIL | CD8+_CD103+ | TGCGCCAGCAGTGAAATGGGGGGGGGCCAAGAGACCCAGTACTTC |
| 285 | 139-TL706-TIL-CD8+_CD103+ | TIL | CD8+_CD103+ | TGTGCCAGCAGTTTCGCGACAGGGGGCCCAGAGACCCAGTACTTC |
| 286 | 139-TL706-TIL-CD8+_CD103+ | TIL | CD8+_CD103+ | TGTGCCAGCACTCCTTGGACAGGGGGCTCCAACTCCTATAATTCAC |
| | | | | CCCTCCACTTT |
| 287 | 139-TL706-TIL-CD8+_CD103+ | TIL | CD8+_CD103+ | TGTGCCAGCAGCTTAGCCGTCGCGGGGGGGGACAATGAGCAGTTCTTC |
| 288 | 139-TL706-TIL-CD8+_CD103+ | TIL | CD8+_CD103+ | TGTGCCAGCAGTTCTAGCGGGGGGCCCTACAATGAGCAGTTCTTC |
| 289 | 139-TL706-TIL-CD8+_CD103+ | TIL | CD8+_CD103+ | TGTGCCAGCAGCCAAGTGCCTAGCGGGGGAGAGACCCAGTACTTC |
| 290 | 139-TL706-TIL-CD8+_CD103+ | TIL | CD8+_CD103+ | TGCAGCGCCGTGGACAGGGACGACGAGCAGTACTTC |
| 291 | 139-TL706-TIL-CD8+_CD103+ | TIL | CD8+_CD103+ | TGTGCCAGCAGCTCATTAGTGCGGGGGGCGCATGGTCAGCCCCAGCATTTT |
| 292 | 139-TL706-TIL-CD8+_CD103+ | TIL | CD8+_CD103+ | TGTGCCACCAGTGACATGGGGAGGGGTGGCGAGCAGTACTTC |
| 293 | 139-TL706-TIL-CD8+_CD103+ | TIL | CD8+_CD103+ | TGTGCCAGCAGCGGGACTAGCAATGAGCAGTTCTTC |
| 294 | 139-TL706-TIL-CD8+_CD103+ | TIL | CD8+_CD103+ | TGTGCCAGCAGTGACTTGTCTTCACCCCTCCACTTT |
| 295 | 139-TL706-TIL-CD8+_CD103+ | TIL | CD8+_CD103+ | TGTGCCAGCAGCGTTACGGACAGGAATCGAGACTACGAGCAGTACTTC |
| 296 | 139-TL706-TIL-CD8+_CD103+ | TIL | CD8+_CD103+ | TGTGCCAGCAGTTACCGGACAGCCTTTGGTAGCACAGATACGCAGTATTTT |
| 297 | 139-TL706-TIL-CD8+_CD103+ | TIL | CD8+_CD103+ | TGTGCCAGCAGTTCTTTGGGGTCCGGGACTGTAGCGAGGGCTAGAGA |
| | | | | CGAGCAGTACTTC |
| 298 | 139-TL706-TIL-CD8+_CD103+ | TIL | CD8+_CD103+ | TGTGCCCAAACGGCGGCAACTAATGAAAAACTGTTTTTT |
| 299 | 139-TL706-TIL-CD8+_CD103+ | TIL | CD8+_CD103+ | TGTGCCGGAAACGGGAACACCGGGGAGCTGTTTTTT |
| 300 | 139-TL706-TIL-CD8+_CD103+ | TIL | CD8+_CD103+ | TGTGCCAGCATCCCGTCCCGACCTGGCACAGATACGCAGTATTTT |
| 301 | 139-TL706-TIL-CD8+_CD103+ | TIL | CD8+_CD103+ | TGTGCCAGCAGTTACTTTCCGGGCGGGGGGAACACTGAAGCTTTCTTT |
| 302 | 139-TL706-TIL-CD8+_CD103+ | TIL | CD8+_CD103+ | TGTGCCAGCAGCTTAGAGTTGCAGGGTCATAATGAAAAACTGTTTTTT |
| 303 | 139-TL706-TIL-CD8+_CD103+ | TIL | CD8+_CD103+ | TGTGCCAGCAGCTCCCCCGTGGTGGAGACCCAGTACTTC |
| 304 | 139-TL706-TIL-CD8+_CD103+ | TIL | CD8+_CD103+ | TGTGCCAGCAGCGAACATGCGAACACTGAAGCTTTCTTT |
| 305 | 139-TL706-TIL-CD8+_CD103+ | TIL | CD8+_CD103+ | TGTGCCAGCACCCAAGGGGGGGCCGGGACCCAGTACTTC |
| 306 | 139-TL706-TIL-CD8+_CD103+ | TIL | CD8+_CD103+ | TGTGCCAGCAGCCATCGAGACAGGGGGGACACTGAAGCTTTCTTT |
| 307 | 139-TL706-TIL-CD8+_CD103+ | TIL | CD8+_CD103+ | TGTGCCAGCAGTTACGGGAGCCTGGGCGGGGAGCTGTTTTTT |
| 308 | 139-TL706-TIL-CD8+_CD103+ | TIL | CD8+_CD103+ | TGTGCCAGTAACTTCCGGGACAGGCCGTACAATGAGCAGTTCTTC |
| 309 | 139-TL706-TIL-CD8+_CD103+ | TIL | CD8+_CD103+ | TGTGCCAGCAGTGAATCTAGCGGGGGGGCAGATACGCAGTATTTT |
| 310 | 139-TL706-TIL-CD8+_CD103+ | TIL | CD8+_CD103+ | TGTGCCAGTAGTAGTCGGAGCTCCTACGAGCAGTACTTC |
| 311 | 139-TL706-TIL-CD8+_CD103+ | TIL | CD8+_CD103+ | TGTGCCAGCAGCTTAGCACAGGGGCCCTCCTACGAGCAGTACTTC |
| 312 | 139-TL706-TIL-CD8+_CD103+ | TIL | CD8+_CD103+ | TGTGCCACCAGGCTGGGACTACAAGGATCTAGCACAGATACGCAGTATTTT |
| 313 | 139-TL706-TIL-CD8+_CD103+ | TIL | CD8+_CD103+ | TGTGCCAGCAGCTTAGGTACTGCGGGGTACACCGGGGAGCTGTTTTTT |
| 314 | 139-TL706-TIL-CD8+_CD103+ | TIL | CD8+_CD103+ | TGCGCCAGCAGTGAAGACGGTATGAACACTGAAGCTTTCTTT |
| 315 | 139-TL706-TIL-CD8+_CD103+ | TIL | CD8+_CD103+ | TGTGCCAGCAACAGGGAGGATGCAGTTAGCACTGAAGCTTTCTTT |
| 316 | 139-TL706-TIL-CD8+_CD103+ | TIL | CD8+_CD103+ | TGTGCCAGCAGTTACTATGCGTCCCCCACTGAAGCTTTCTTT |
| 317 | 139-TL706-TIL-CD8+_CD103+ | TIL | CD8+_CD103+ | TGTGCCAGCAGCCGAGTTGGAACCGGGGAGCTGTTTTTT |
| 318 | 139-TL706-TIL-CD8+_CD103+ | TIL | CD8+_CD103+ | TGTGCCAGCAGTTACTCCAAAAGGAACCGATCACCCCTCCACTTT |
| 319 | 139-TL706-TIL-CD8+_CD103+ | TIL | CD8+_CD103+ | TGTGCCAGCAGTTCCACACCGGACCTCTACAATGAGCAGTTCTTC |
| 320 | 139-TL706-TIL-CD8+_CD103+ | TIL | CD8+_CD103+ | TGCGCCAGCAGCCAAGACCCCCCAGGGACAACATATATCGATAAT |
| | | | | TCACCCCTCCACTTT |
| 321 | 139-TL706-TIL-CD8+_CD103+ | TIL | CD8+_CD103+ | TGTGCCAGCAGCCCAGGGGTCGCTGGCTACACCTTC |
| 322 | 139-TL706-TIL-CD8+_CD103+ | TIL | CD8+_CD103+ | TGTGCCAGCAGTGAACCTAGGACAGGGGGAAACAATGAGCAGTTCTTC |
| 323 | 139-TL706-TIL-CD8+_CD103+ | TIL | CD8+_CD103+ | TGTGCCAGCAGTTTATTCGCAATAGAGCAGGGGAACACCGGGGAGCTG |
| | | | | TTTTTT |
| 324 | 140-TL706-TIL-CD8+_CD103− | TIL | CD8+_CD103− | TGTGCCAGCAGTGACCGGGACAGGGCCACAGATACGCAGTATTTT |
| 325 | 140-TL706-TIL-CD8+_CD103− | TIL | CD8+_CD103− | TGTGCCAGCAGCCACCCCGGACTAGCGGGGGGCCGGTACCAAGAGAC |
| | | | | CCAGTACTTC |
| 326 | 140-TL706-TIL-CD8+_CD103− | TIL | CD8+_CD103− | TGTGCCAGCAGCCCTGAGACTGAAGCTTTCTTT |
| 327 | 140-TL706-TIL-CD8+_CD103− | TIL | CD8+_CD103− | TGTGCCAGCAGTGAAGAAACAGTCTCTAATGAAAAACTGTTTTTT |
| 328 | 140-TL706-TIL-CD8+_CD103− | TIL | CD8+_CD103− | TGTGCCAGCAGTCCACATCTGGGACAGATACCTTACAATGAGCAGTTCTTC |
| 329 | 140-TL706-TIL-CD8+_CD103− | TIL | CD8+_CD103− | TGTGCCATCAGTGAGACCCGACAGGGAGGCGTGAGGACTGAAGCTTTCTTT |
| 330 | 140-TL706-TIL-CD8+_CD103− | TIL | CD8+_CD103− | TGTGCCAGCAGCCAAGAGGCCCTAGCTCAGAACAATGAGCAGTTCTTC |
| 331 | 140-TL706-TIL-CD8+_CD103− | TIL | CD8+_CD103− | TGTGCCACCAGTGGGCGGGTGGCAGCCCGATACTACAATGAGCAGTTCTTC |
| 332 | 140-TL706-TIL-CD8+_CD103− | TIL | CD8+_CD103− | TGTGCCAGCAGCCCATTTATAGAGGCCTTTACGCAGTATTTT |
| 333 | 140-TL706-TIL-CD8+_CD103− | TIL | CD8+_CD103− | TGTGCCAGCAGCTCAAATTTGATGACAGAAGCAAAAGCTAACTATGG |
| | | | | CTACACCTTC |
| 334 | 140-TL706-TIL-CD8+_CD103− | TIL | CD8+_CD103− | TGTGCCAGCGGCACTTACGGGGCAAACTACGAGCAGTACTTC |
| 335 | 140-TL706-TIL-CD8+_CD103− | TIL | CD8+_CD103− | TGTGCCAGCAGCCCTCGACTAGCGGGGGGCCGTCAAAGCACA |
| | | | | GATACGCAGTATTTT |
| 336 | 140-TL706-TIL-CD8+_CD103− | TIL | CD8+_CD103− | TGTGCCAGTAGTACCCCCCCAGGGATGGGGGTCGCGACTAAT |
| | | | | GAAAAACTGTTTTTT |
| 337 | 140-TL706-TIL-CD8+_CD103− | TIL | CD8+_CD103− | TGTGCCAGCAGTTCCGGACGTCTCTCTGGGGCCAACGTCCTGACTTTC |
| 338 | 140-TL706-TIL-CD8+_CD103− | TIL | CD8+_CD103− | TGTGCCAGCAGCTTAGGGCCCGGGTACCAAGAGACCCAGTACTTC |
| 339 | 140-TL706-TIL-CD8+_CD103− | TIL | CD8+_CD103− | TGTGCCAGGACCCACGACTACCTGGATAGCGGGGGGGCCGCAGAT |
| | | | | ACGCAGTATTTT |
| 340 | 140-TL706-TIL-CD8+_CD103− | TIL | CD8+_CD103− | TGCGCCAGCAGCTTTGGAGGGCAGGAGTACTCTGGAAACACCATATATTTT |
| 341 | 140-TL706-TIL-CD8+_CD103− | TIL | CD8+_CD103− | TGTGCCAGCAGCCAGGAAATGACAGGGTTGTCCTCCACAGATAC |
| | | | | GCAGTATTTT |
| 342 | 140-TL706-TIL-CD8+_CD103− | TIL | CD8+_CD103− | TGTGCCAGCAGCCAAGATGGGACAGGGGCCTACGAGCAGTACTTC |
| 343 | 140-TL706-TIL-CD8+_CD103− | TIL | CD8+_CD103− | TGTGCCAGCAGCTTAGGAATTCCGCGGGGTACAGTAGAGCTGTTTTTT |
| 344 | 140-TL706-TIL-CD8+_CD103− | TIL | CD8+_CD103− | TGTGCCAGCAGTGAAGAGGCGGCGGGAACAGATACGCAGTATTTT |
| 345 | 140-TL706-TIL-CD8+_CD103− | TIL | CD8+_CD103− | TGTGCCAGCAGTTCCCGAGGGACGGACGCAGATACGCAGTATTTT |
| 346 | 140-TL706-TIL-CD8+_CD103− | TIL | CD8+_CD103− | TGCGCCAGCAGTGAAGACGGTATGAACACTGAAGCTTTCTTT |
| 347 | 140-TL706-TIL-CD8+_CD103− | TIL | CD8+_CD103− | TGCGCCAGCAGCTCCCCGACAGGTATGAACACTGAAGCTTTCTTT |
| 348 | 140-TL706-TIL-CD8+_CD103− | TIL | CD8+_CD103− | TGTGCCAGCAGTTCGTACAGGACAAATGAAAAACTGTTTTTT |
| 349 | 140-TL706-TIL-CD8+_CD103− | TIL | CD8+_CD103− | TGCGCCAGCAGCTTGAACAACGCGGGGGGCTGGGACAATGAGCAGTTCTTC |
| 350 | 140-TL706-TIL-CD8+_CD103− | TIL | CD8+_CD103− | TGTGCCAGCAGCTTGGCCGGCCAGCGGGGGGGCCGTAGGACAG |
| | | | | ATACGCAGTATTTT |
| 351 | 140-TL706-TIL-CD8+_CD103− | TIL | CD8+_CD103− | TGTGCCAGCAGCCCGACAGGGATTGAAGCTTTCTTT |
| 352 | 140-TL706-TIL-CD8+_CD103− | TIL | CD8+_CD103− | TGTGCCAGCAGTTGGACAGGGGGCCAAGAGACCCAGTACTTC |
| 353 | 140-TL706-TIL-CD8+_CD103− | TIL | CD8+_CD103− | TGTGCCAGCAAGGGACAGGGGATTTATGGCTACACCTTC |
| 354 | 140-TL706-TIL-CD8+_CD103− | TIL | CD8+_CD103− | TGTGCCAGCAGTTTGTCCGGGGCCTCCTATGGCTACACCTTC |
| 355 | 140-TL706-TIL-CD8+_CD103− | TIL | CD8+_CD103− | TGTGCCAGCAGTTTTAAACAGGATTACTATGGCTACACCTTC |
| 356 | 140-TL706-TIL-CD8+_CD103− | TIL | CD8+_CD103− | TGTGCCACCCAAAGGACAGGGGGTATCGCTGAAGCTTTCTTT |
| 357 | 140-TL706-TIL-CD8+_CD103− | TIL | CD8+_CD103− | TGTGCCACCGACTTAGATGAGGGCTTGAACACTGAAGCTTTCTTT |
| 358 | 140-TL706-TIL-CD8+_CD103− | TIL | CD8+_CD103− | TGTGCCACCAGTCGGACAGGGGCCGATGCGGAGCAGTTCTTC |
| 359 | 140-TL706-TIL-CD8+_CD103− | TIL | CD8+_CD103− | TGCGCCAGCAGCTACGACAGGTTCGACGAGCAGTACTTC |
| 360 | 140-TL706-TIL-CD8+_CD103− | TIL | CD8+_CD103− | TGTGCCGGACGGGACAGCCTGGACTATGGCTACACCTTC |
| 361 | 140-TL706-TIL-CD8+_CD103− | TIL | CD8+_CD103− | TGTGCCAGCAGCTTGACAGGTCCCGGCAATTCACCCCTCCACTTT |
| 362 | 140-TL706-TIL-CD8+_CD103− | TIL | CD8+_CD103− | TGTGCCAGCAGCCCGTCGACAGGGCGCTCTGGAAACACCATATATTTT |
| 363 | 140-TL706-TIL-CD8+_CD103− | TIL | CD8+_CD103− | TGCGCCAGCAGCTTGGGACTAAATCACGAGACCCAGTACTTC |
| 364 | 140-TL706-TIL-CD8+_CD103− | TIL | CD8+_CD103− | TGCGCCAGCAGCCAAGGTGGCGAGCAGTACTTC |
| 365 | 140-TL706-TIL-CD8+_CD103− | TIL | CD8+_CD103− | TGTGCCAGCAGCCCAGGGGAGGTGGCAGATACGCAGTATTTT |
| 366 | 140-TL706-TIL-CD8+_CD103− | TIL | CD8+_CD103− | TGCGCCAGCAGCCAATCACGGACTAGCGGGGGGCGGGGAGAGCAGTTCTTC |
| 367 | 140-TL706-TIL-CD8+_CD103− | TIL | CD8+_CD103− | TGTGCCAGCAAACCCAAAAAGGGACAGGGGAACACCGGGGAGCTGTTTTTT |
| 368 | 140-TL706-TIL-CD8+_CD103− | TIL | CD8+_CD103− | TGTGCCAGCAGTTCCAGCCCTATATCGGAGTTCTTC |
| 369 | 140-TL706-TIL-CD8+_CD103− | TIL | CD8+_CD103− | TGCGCCAGCAGCCGAGAAGACACGGCCGTGGATGGCTACACCTTC |
| 370 | 140-TL706-TIL-CD8+_CD103− | TIL | CD8+_CD103− | TGTGCCAGCAGCCAAAGCGGGGGTCTAGAAGATACGCAGTATTTT |
| 371 | 140-TL706-TIL-CD8+_CD103− | TIL | CD8+_CD103− | TGCGCCAGCAGCACGCCGGGACAGGGACTCTACGAGCAGTACTTC |
| 372 | 140-TL706-TIL-CD8+_CD103− | TIL | CD8+_CD103− | TGTGCCAGCAGCTTAGTCCGATTAATTAGGACTAGCGGCGACTAC |
| | | | | GAGCAGTACTTC |
| 373 | 140-TL706-TIL-CD8+_CD103− | TIL | CD8+_CD103− | TGTGCCAGCAGTTTAATAGGGACAGGGTTGACCGGGGAGCTGTTTTTT |
| 374 | 140-TL706-TIL-CD8+_CD103− | TIL | CD8+_CD103− | TGTGCCAGCAGCCTGCAGGGTGGTCGATATGGCTACACCTTC |
| 375 | 140-TL706-TIL-CD8+_CD103− | TIL | CD8+_CD103− | TGCGCCAGCAGCCACGGACAGGGGCTGGTCACTGAAGCTTTCTTT |
| 376 | 140-TL706-TIL-CD8+_CD103− | TIL | CD8+_CD103− | TGTGCCAGCAGTGGCACAGGGGCCGAGCAGTACTTC |
| 377 | 140-TL706-TIL-CD8+_CD103− | TIL | CD8+_CD103− | TGTGCCAGCAGTTTCCCAGTGACGCAGGGAAGGAAC |
| | | | | ACCGGGGAGCTGTTTTTT |
| 378 | 140-TL706-TIL-CD8+_CD103− | TIL | CD8+_CD103− | TGTGCCAGCAGCGGGACAGGGACTGCCTACGAGCAGTACTTC |
| 379 | 140-TL706-TIL-CD8+_CD103− | TIL | CD8+_CD103− | TGCGCCAGCAGCCAAGACCCCCCAGGGACAACATATATCGATAA |
| | | | | TTCACCCCTCCACTTT |
| 380 | 140-TL706-TIL-CD8+_CD103− | TIL | CD8+_CD103− | TGCGCCAGCAGCCAAGACCAGCCAAGATATAGCAATCAGCCCCAGCATTTT |
| 381 | 140-TL706-TIL-CD8+_CD103− | TIL | CD8+_CD103− | TGCGCCAGCAGCTTCGGGACAGGGATGGCAGATACGCAGTATTTT |
| 382 | 140-TL706-TIL-CD8+_CD103− | TIL | CD8+_CD103− | TGTGCCAGCAGTTGGGGCGGGGGGCATACAGATACGCAGTATTTT |
| 383 | 140-TL706-TIL-CD8+_CD103− | TIL | CD8+_CD103− | TGTGCCAGCAGTGATTCGGCCAGCAATTCACCCCTCCACTTT |
| 384 | 140-TL706-TIL-CD8+_CD103− | TIL | CD8+_CD103− | TGTGCCAGCAGCGTAATCAAGGGGGCGGAGGAGACCCAGTACTTC |
| 385 | 140-TL706-TIL-CD8+_CD103− | TIL | CD8+_CD103− | TGCGCCAGCAGCTCGGCGACTAGCGGGGGCCCTAATGGATACAAT |
| | | | | GAGCAGTTCTTC |
| 386 | 140-TL706-TIL-CD8+_CD103− | TIL | CD8+_CD103− | TGTGCCAGCAGCTTCGGGACACCCACTGAAGCTTTCTTT |
| 387 | 140-TL706-TIL-CD8+_CD103− | TIL | CD8+_CD103− | TGTGCCAGTGGACAGGGGGTGAAGTACGAGCAGTACTTC |
| 388 | 140-TL706-TIL-CD8+_CD103− | TIL | CD8+_CD103− | TGTGCCAGCACCTCTCTCCCCCAGGGGGATGGCTACGAGCAGTACTTC |
| 389 | 140-TL706-TIL-CD8+_CD103− | TIL | CD8+_CD103− | TGTGCCAGCAGTGAGAGATGGGGACCCCTCTCCTACGAGCAGTACTTC |
| 390 | 140-TL706-TIL-CD8+_CD103− | TIL | CD8+_CD103− | TGTGCCAGTAGTATAGCGGAGAACACTGAAGCTTTCTTT |
| 391 | 140-TL706-TIL-CD8+_CD103− | TIL | CD8+_CD103− | TGTGCCAGCAGTTTACAGGGAAACACTGAAGCTTTCTTT |
| 392 | 140-TL706-TIL-CD8+_CD103− | TIL | CD8+_CD103− | TGTGCCAGCTCACCCTGGGGACTAGCGGGGGCCGAAGAGACCCAGTACTTC |
| 393 | 140-TL706-TIL-CD8+_CD103− | TIL | CD8+_CD103− | TGTGCCAGCAGCCCTAGCGACTCTAGCACAGATACGCAGTATTTT |
| 394 | 140-TL706-TIL-CD8+_CD103− | TIL | CD8+_CD103− | TGTGCCAGCAGCAAGAACACCGGGGAGCTGTTTTTT |
| 395 | 140-TL706-TIL-CD8+_CD103− | TIL | CD8+_CD103− | TGTGCCAGCAGTTACCGGCAGGGACTTTGGGGCGAGCAGTACTTC |
| 396 | 140-TL706-TIL-CD8+_CD103− | TIL | CD8+_CD103− | TGTGCCACCAGCAGAGGACTAGACGGTTACGAGCAGTACTTC |
| 397 | 140-TL706-TIL-CD8+_CD103− | TIL | CD8+_CD103− | TGTGCCAGTAGCGACAGGATTGGCAACACTGAAGCTTTCTTT |
| 398 | 140-TL706-TIL-CD8+_CD103− | TIL | CD8+_CD103− | TGTGCCAGCAGGGCAAGACAGGCTCGAGTCCATGAGCAGTTCTTC |
| 399 | 140-TL706-TIL-CD8+_CD103− | TIL | CD8+_CD103− | TGCGCCAGCAGACATGGACAGGGGGATAATTCACCCCTCCACTTT |
| 400 | 140-TL706-TIL-CD8+_CD103− | TIL | CD8+_CD103− | TGTGCCAGCAGTTACGATCTAGCGGGGGGAGACGAGCAGTACTTC |
| 401 | 140-TL706-TIL-CD8+_CD103− | TIL | CD8+_CD103− | TGTGCCAGCAGCGTAGGTGGGGGACTAGCCCCTTCGGTGTCCTAC |
| | | | | AATGAGCAGTTCTTC |
| 402 | 140-TL706-TIL-CD8+_CD103− | TIL | CD8+_CD103− | TGCGCCAGCAGCCAAGAGGGCAGGGGCTGGACTGAAGCTTTCTTT |
| 403 | 140-TL706-TIL-CD8+_CD103− | TIL | CD8+_CD103− | TGTGCCAGCACCCCCTCCCGTCTGGGGGCCCGGCCCCCAGATACG |
| | | | | CAGTATTTT |
| 404 | 140-TL706-TIL-CD8+_CD103− | TIL | CD8+_CD103− | TGTGCCAGTAGTACCTGGGACAGGGGGAATAGTCTCCCTGAAGCTTTCTTT |
| 405 | 140-TL706-TIL-CD8+_CD103− | TIL | CD8+_CD103− | TGTGCCAGCGGGAAACAGGAGAACTATGGCTACACCTTC |
| 406 | 140-TL706-TIL-CD8+_CD103− | TIL | CD8+_CD103− | TGTGCCAGCAGCTTGTTTCCAGGGGGTAAAGGGGGATTTTATGAAAA |
| | | | | ACTGTTTTTT |
| 407 | 140-TL706-TIL-CD8+_CD103− | TIL | CD8+_CD103− | TGTGCCAGTAGTATACGCTCGGCTTACGAGCAGTACTTC |
| 408 | 140-TL706-TIL-CD8+_CD103− | TIL | CD8+_CD103− | TGTGCCAGCAGTTGGGGCAGCTACGAGCAGTACTTC |
| 409 | 140-TL706-TIL-CD8+_CD103− | TIL | CD8+_CD103− | TGTGCCAGCGGGACGGGGACGGACACTGAAGCTTTCTTT |
| 410 | 140-TL706-TIL-CD8+_CD103− | TIL | CD8+_CD103− | TGCAGCGTGCCTTGGACAGGTGCTTATGGCTACACCTTC |
| 411 | 140-TL706-TIL-CD8+_CD103− | TIL | CD8+_CD103− | TGTGCCAGCAGTGTACCGGGCCGGAGCACCGGGGAGCTGTTTTTT |
| 412 | 140-TL706-TIL-CD8+_CD103− | TIL | CD8+_CD103− | TGTGCCAGCAGTTTAGACCAGGGATCTGGGTCACCCCTCCACTTT |
| 413 | 140-TL706-TIL-CD8+_CD103− | TIL | CD8+_CD103− | TGTGCCAGCAGCTCCCCCGCCCGTGAGCAGTTCTTC |
| 414 | 140-TL706-TIL-CD8+_CD103− | TIL | CD8+_CD103− | TGCAGTGCTAGTGGGGCTAGGTTTAACTACGAGCAGTACTTC |
| 415 | 140-TL706-TIL-CD8+_CD103− | TIL | CD8+_CD103− | TGTGCCAGTAGTACCGGGGCATATGGCTACACCTTC |
| 416 | 140-TL706-TIL-CD8+_CD103− | TIL | CD8+_CD103− | TGTGCCAGCAGCCCCCCCGTAGGGACCTCCTACGAGCAGTACTTC |
| 417 | 140-TL706-TIL-CD8+_CD103− | TIL | CD8+_CD103− | TGTGCCAGCAGTGAGACAGGGAGGGAAGAGACCCAGTACTTC |
| 418 | 140-TL706-TIL-CD8+_CD103− | TIL | CD8+_CD103− | TGTGCCAGCAGCGCTCGACAGGGACCCTCCAATGAGCAGTTCTTC |
| 419 | 140-TL706-TIL-CD8+_CD103− | TIL | CD8+_CD103− | TGTGCCAGCAGCTTGCCGCAGCCAGGGTGGGACCAAGAGACCCAGTACTTC |
| 420 | 140-TL706-TIL-CD8+_CD103− | TIL | CD8+_CD103− | TGTGCCAGCAGCTTAATGGGGGGCACAGATACGCAGTATTTT |
| 421 | 140-TL706-TIL-CD8+_CD103− | TIL | CD8+_CD103− | TGTGCCAGCAGTCCGGGGGGAGGGTGGCCTTTGAATGAGCAGTTCTTC |
| 422 | 140-TL706-TIL-CD8+_CD103− | TIL | CD8+_CD103− | TGTGCCAGCAGCGTAGGCGCTCTGGCCAACACTGAAGCTTTCTTT |
| 423 | 140-TL706-TIL-CD8+_CD103− | TIL | CD8+_CD103− | TGTGCCAGCAGTACAACAGGTACCCATAGCAATCAGCCCCAGCATTTT |
| 424 | 140-TL706-TIL-CD8+_CD103− | TIL | CD8+_CD103− | TGTGCCAGTAGTATAACAGGCGTCCGCACAGATACGCAGTATTTT |
| 425 | 140-TL706-TIL-CD8+_CD103− | TIL | CD8+_CD103− | TGCGCCAGCAGCTTGGGGGGGGTCAGCACAGATACGCAGTATTTT |
| 426 | 140-TL706-TIL-CD8+_CD103− | TIL | CD8+_CD103− | TGTGCCAGTAGTATACGGTCCTCCTACGAGCAGTACTTC |
| 427 | 140-TL706-TIL-CD8+_CD103− | TIL | CD8+_CD103− | TGTGCCAGCAGGCAGACAGGGAGAAATCAGCCCCAGCATTTT |
| 428 | 140-TL706-TIL-CD8+_CD103− | TIL | CD8+_CD103− | TGTGCCAGCAGCTTGGGTGACGAGACCACTGAAGCTTTCTTT |
| 429 | 140-TL706-TIL-CD8+_CD103− | TIL | CD8+_CD103− | TGTGCCAGCAGCGTCACAGGACAGGGCTACAATGAGCAGTTCTTC |
| 430 | 140-TL706-TIL-CD8+_CD103− | TIL | CD8+_CD103− | TGTGCCAGCAGCGTAGGGGGGGAGAACACCGGGGAGCTGTTTTTT |
| 431 | 140-TL706-TIL-CD8+_CD103− | TIL | CD8+_CD103− | TGTGCCAGCAGCTTAGCGCCGACAGGGTGGGGATATAATTCACCC |
| | | | | CTCCACTTT |
| 432 | 140-TL706-TIL-CD8+_CD103− | TIL | CD8+_CD103− | TGTGCCACCAGGGGACGCGGGGAATCGTACAATGAGCAGTTCTTC |
| 433 | 140-TL706-TIL-CD8+_CD103− | TIL | CD8+_CD103− | TGCGCCAGCAGCCCCGGGGGGAGCTCCTACAATGAGCAGTTCTTC |
| 434 | 140-TL706-TIL-CD8+_CD103− | TIL | CD8+_CD103− | TGCGCCAGCAGCTGGACAGGGGCGGGGAACACTGAAGCTTTCTTT |
| 435 | 140-TL706-TIL-CD8+_CD103− | TIL | CD8+_CD103− | TGTGCCAGCAGTTACCGACGGGTTCCTGGGGCCAACGTCCTGACTTTC |
| 436 | 140-TL706-TIL-CD8+_CD103− | TIL | CD8+_CD103− | TGCGCCAGCAGCCAAGATGAGTATGAGCAGTTCTTC |
| 437 | 140-TL706-TIL-CD8+_CD103− | TIL | CD8+_CD103− | TGTGCCAGCAGCTTATCGGGTGGTGGTGAAGCTTTCTTT |
| 438 | 140-TL706-TIL-CD8+_CD103− | TIL | CD8+_CD103− | TGCGCCAGCAGCCAGGGGACAGGTTTCGAAAAACTGTTTTTT |
| 439 | 140-TL706-TIL-CD8+_CD103− | TIL | CD8+_CD103− | TGTGCCAGCAGCTTAATCTCCGGGGGTGGCACCGGGGAGCTGTTTTTT |
| 440 | 140-TL706-TIL-CD8+_CD103− | TIL | CD8+_CD103− | TGTGCCAGCAGTGCGGTGGGGACAGGACTCACCGGGGAGCTGTTTTTT |
| 441 | 140-TL706-TIL-CD8+_CD103− | TIL | CD8+_CD103− | TGTGCCAGCAGCTTAGCAGGAAACGAGCAGTACTTC |
| 442 | 140-TL706-TIL-CD8+_CD103− | TIL | CD8+_CD103− | TGTGCCAGCAGCTTAGCGAATCTAGCTCTCAATGAGCAGTTCTTC |
| 443 | 140-TL706-TIL-CD8+_CD103− | TIL | CD8+_CD103− | TGTGCCAGCAGCGTAGGGTTTCGACCCAATAGCAATCAGCCCCAGCATTTT |
| 444 | 140-TL706-TIL-CD8+_CD103− | TIL | CD8+_CD103− | TGCGCCAGCAGCTTGGTTGGGTCGCACGAGCAGTACTTC |
| 445 | 140-TL706-TIL-CD8+_CD103− | TIL | CD8+_CD103− | TGTGCCAGCAGCTTGGATGCAGGGGCCGCCTACGAGCAGTACTTC |
| 446 | 140-TL706-TIL-CD8+_CD103− | TIL | CD8+_CD103− | TGTGCCAGCAGCTTAACGGGGACCAGGGGAGAGACCCAGTACTTC |
| 447 | 140-TL706-TIL-CD8+_CD103− | TIL | CD8+_CD103− | TGTGCCAGCAGCGTTCTTGAAGAGACCCAGTACTTC |
| 448 | 140-TL706-TIL-CD8+_CD103− | TIL | CD8+_CD103− | TGTGCCAGCAGCTCGACTGCGGGGGCCCCCCGGATCTTC |
| 449 | 140-TL706-TIL-CD8+_CD103− | TIL | CD8+_CD103− | TGCAGTGCTAGGGTCAAAGGGACAGGGGGTCATCAGCCCCAGCATTTT |
| 450 | 140-TL706-TIL-CD8+_CD103− | TIL | CD8+_CD103− | TGTGCCAGCAGCCCCCACGGCAGCCAAGAGACCCAGTACTTC |
| 451 | 140-TL706-TIL-CD8+_CD103− | TIL | CD8+_CD103− | TGTGCCAGCAGTGTTAGCGGGGGCAGGTTGGACACCGGGGAGCTGTTTTTT |
| 452 | 140-TL706-TIL-CD8+_CD103− | TIL | CD8+_CD103− | TGCGCCAGCAGCCATAGCGCGGGGGCTAATGAGCAGTTCTTC |
| 453 | 140-TL706-TIL-CD8+_CD103− | TIL | CD8+_CD103− | TGTGCCCAAACGGCGGCAACTAATGAAAAACTGTTTTTT |
| 454 | 140-TL706-TIL-CD8+_CD103− | TIL | CD8+_CD103− | TGTGCCAGCAGAGAGGAGACAGGGCCCAACTACTACGAGCAGTACTTC |
| 455 | 140-TL706-TIL-CD8+_CD103− | TIL | CD8+_CD103− | TGCAGCGTTGGTATGAGACAGGGTGGTTTAGGCACAGATACGCAGTATTTT |
| 456 | 140-TL706-TIL-CD8+_CD103− | TIL | CD8+_CD103− | TGTGCCAGCAGTCCGGGGGGGTCCTACGAGCAGTACTTC |
| 457 | 140-TL706-TIL-CD8+_CD103− | TIL | CD8+_CD103− | TGTGCCAGCAGCGCTGGAGGGACAGGGGCCGCTGAAGCTTTCTTT |
| 458 | 140-TL706-TIL-CD8+_CD103− | TIL | CD8+_CD103− | TGTGCCAGCAGTTATGGGCGGGGGGGCCACAATGAGCAGTTCTTC |
| 459 | 140-TL706-TIL-CD8+_CD103− | TIL | CD8+_CD103− | TGTGCCAGCAGTTTTGTCGCCGGGACAGATGAGCAGTACTTC |
| 460 | 140-TL706-TIL-CD8+_CD103− | TIL | CD8+_CD103− | TGTGCCAGCAGCTTAGGCAGGGGCTATTACGAGCAGTACTTC |
| 461 | 140-TL706-TIL-CD8+_CD103− | TIL | CD8+_CD103− | TGTGCCAGCAGCTTCCCCCAAGAGACCATGAACACTGAAGCTTTCTTT |
| 462 | 140-TL706-TIL-CD8+_CD103− | TIL | CD8+_CD103− | TGTGCCAGCAGCTTAGTCGCAGCCACAGATACGCAGTATTTT |
| 463 | 140-TL706-TIL-CD8+_CD103− | TIL | CD8+_CD103− | TGTGCCAGCAGTTGGACACGGAACACTGAAGCTTTCTTT |
| 464 | 140-TL706-TIL-CD8+_CD103− | TIL | CD8+_CD103− | TGTGCCAGCAGCTTTTCTGGGACAGGGGACACCGGGGAGCTGTTTTTT |
| 465 | 140-TL706-TIL-CD8+_CD103− | TIL | CD8+_CD103− | TGTGCCAGCTCACCAGGGGGACAGGGGACGGTTAACTATGGCTACACCTTC |
| 466 | 140-TL706-TIL-CD8+_CD103− | TIL | CD8+_CD103− | TGCAGTGCTAGGGACAGGGTGGGTAATGGCTACACCTTC |
| 467 | 140-TL706-TIL-CD8+_CD103− | TIL | CD8+_CD103− | TGCGCCAGCAGCTTATTTGGGGCCTACGAGCAGTACTTC |
| 468 | 140-TL706-TIL-CD8+_CD103− | TIL | CD8+_CD103− | TGTGCCAGCAGCTTCACCGGGACAAGCTACGAGCAGTACTTC |
| 469 | 140-TL706-TIL-CD8+_CD103− | TIL | CD8+_CD103− | TGTGCCAGCAGCTTGATGGGCGGGAGAAACACCGGGGAGCTGTTTTTT |
| 470 | 140-TL706-TIL-CD8+_CD103− | TIL | CD8+_CD103− | TGTGCCAGCAGCGTATACGGGGGTGAGGGAGAGACCCAGTACTTC |
| 471 | 140-TL706-TIL-CD8+_CD103− | TIL | CD8+_CD103− | TGTGCCAGCAGTTGGGGGAGAGCAACTAATGAAAAACTGTTTTTT |
| 472 | 140-TL706-TIL-CD8+_CD103− | TIL | CD8+_CD103− | TGTGCCAGCAGTTCTGGACGGCTCTCTGGGGCCAACGTCCTGACTTTC |
| 473 | 140-TL706-TIL-CD8+_CD103− | TIL | CD8+_CD103− | TGCAGTGCTAGAGATAGGGAGTCATCGAACACCGGGGAGCTGTTTTTT |
| 474 | 140-TL706-TIL-CD8+_CD103− | TIL | CD8+_CD103− | TGTGCCAGCAGCCCCGGACGACTAGCCGATAGCACAGATACGCAGTATTTT |
| 475 | 140-TL706-TIL-CD8+_CD103− | TIL | CD8+_CD103− | TGTGCCAGCAGTTTATCGCGGCGGGAAAAAGAGACCCAGTACTTC |
| 476 | 140-TL706-TIL-CD8+_CD103− | TIL | CD8+_CD103− | TGCGCCAGCACTATAGCTCTCGGGACAGGGTTCGGCTACACCTTC |
| 477 | 140-TL706-TIL-CD8+_CD103− | TIL | CD8+_CD103− | TGTGCCAGCAGCTCACGACTAGCGGGGGTTGAGCAGTACTTC |
| 478 | 140-TL706-TIL-CD8+_CD103− | TIL | CD8+_CD103− | TGCAGTGCTAGAGTTAGGCAGGGGTATGGCTACACCTTC |
| 479 | 140-TL706-TIL-CD8+_CD103− | TIL | CD8+_CD103− | TGTGCCAGCACCCGGAGAGGCAGGCCAGCCTACGAGCAGTACTTC |
| 480 | 140-TL706-TIL-CD8+_CD103− | TIL | CD8+_CD103− | TGTGCCAGTAGTATAATTTGGGGGACCGGGGAGCTGTTTTTT |
| 481 | 140-TL706-TIL-CD8+_CD103− | TIL | CD8+_CD103− | TGTGCCAGCAGCACGACGGGGGGCTACAATGAGCAGTTCTTC |
| 482 | 140-TL706-TIL-CD8+_CD103− | TIL | CD8+_CD103− | TGCGCCAGCAGCCACCCCGGACTGAGCTACGAGCAGTACTTC |
| 483 | 140-TL706-TIL-CD8+_CD103− | TIL | CD8+_CD103− | TGTGCCAGCACCAATACAGGGGCCTATGGCTACACCTTC |
| 484 | 140-TL706-TIL-CD8+_CD103− | TIL | CD8+_CD103− | TGTGCCAGCAGCCCCGCTAGCGGGGACTCCTACGAGCAGTACTTC |
| 485 | 140-TL706-TIL-CD8+_CD103− | TIL | CD8+_CD103− | TGTGCCAGTAGCCCCGGGGGAGGTGAAAAACTGTTTTTT |
| 486 | 140-TL706-TIL-CD8+_CD103− | TIL | CD8+_CD103− | TGTGCCAGCAGCTTAGGTGGGACGGAGCGATACACCTTC |
| 487 | 140-TL706-TIL-CD8+_CD103− | TIL | CD8+_CD103− | TGTGCCAGTAGTATAGCAGGGGCAGATACGCAGTATTTT |
| 488 | 140-TL706-TIL-CD8+_CD103− | TIL | CD8+_CD103− | TGTGCCAGCAGCTTTTCTCAGGCTCAATATGGCTACACCTTC |
| 489 | 140-TL706-TIL-CD8+_CD103− | TIL | CD8+_CD103− | TGTGCCAGCAGTTTATCGCAGGGGGTTCTTGAGACCCAGTACTTC |
| 490 | 140-TL706-TIL-CD8+_CD103− | TIL | CD8+_CD103− | TGCGCCAGCAGCTTGGAGGCAAGCCCTGAAGCTTTCTTT |
| 491 | 140-TL706-TIL-CD8+_CD103− | TIL | CD8+_CD103− | TGTGCCATCAGTCAAGAGGGGAACATCTACGAGCAGTACTTC |
| 492 | 140-TL706-TIL-CD8+_CD103− | TIL | CD8+_CD103− | TGTGCCAGCAGTTTGGGCAGTAACACTGAAGCTTTCTTT |
| 493 | 140-TL706-TIL-CD8+_CD103− | TIL | CD8+_CD103− | TGTGCCAGCAGCAGCTCCTACGAGCAGTACTTC |
| 494 | 140-TL706-TIL-CD8+_CD103− | TIL | CD8+_CD103− | TGTGCCAGTCAGGGGACAGGGGGAATCTACGAGCAGTACTTC |
| 495 | 140-TL706-TIL-CD8+_CD103− | TIL | CD8+_CD103− | TGTGCCAGCAGCTCGGGACAGGGGACGTGGAACTATGGCTACACCTTC |
| 496 | 140-TL706-TIL-CD8+_CD103− | TIL | CD8+_CD103− | TGTGCCAGCAGTTCTGTACAGGGTGCGAACCTCCCGGGGGAAAAACTG |
| | | | | TTTTTT |
| 497 | 140-TL706-TIL-CD8+_CD103− | TIL | CD8+_CD103− | TGTGCCAGCAGTTTACTTAGTCCTAGCGGGGGCCAAGAGACCCAGTACTTC |
| 498 | 140-TL706-TIL-CD8+_CD103− | TIL | CD8+_CD103− | TGTGCCAGCAGCTTAAGTTCCAGGGATTATGTGGGGTACACTGAAGCTTTC |
| | | | | TTT |
| 499 | 151-TL722-TIL-CD8+_CD103+ | TIL | CD8+_CD103+ | TGTGCCAGCTCACCACCTGACAACCCAACTAATGAAAAACTGTTTTTT |
| 500 | 151-TL722-TIL-CD8+_CD103+ | TIL | CD8+_CD103+ | TGCAGCGTTGTGCCTCCCGGGGGCCCAGATACGCAGTATTTT |
| 501 | 151-TL722-TIL-CD8+_CD103+ | TIL | CD8+_CD103+ | TGTGCCAGCAGCCAAACTGCAGGGAGAGATACGCAGTATTTT |
| 502 | 151-TL722-TIL-CD8+_CD103+ | TIL | CD8+_CD103+ | TGTGCCAGCAGTTACTCGGATCACCGTCTTAACTATGGCTACACCTTC |
| 503 | 151-TL722-TIL-CD8+_CD103+ | TIL | CD8+_CD103+ | TGTGCCAGCACCGGGGGAGACCGCTACTATGGCTACACCTTC |
| 504 | 151-TL722-TIL-CD8+_CD103+ | TIL | CD8+_CD103+ | TGCGCCAGCAGCCAAGATGCCAATGAAGCTTTCTTT |
| 505 | 151-TL722-TIL-CD8+_CD103+ | TIL | CD8+_CD103+ | TGTGCCAGCAGCTATCACTGGAATGAGCAGTTCTTC |
| 506 | 151-TL722-TIL-CD8+_CD103+ | TIL | CD8+_CD103+ | TGCGCCAGTAAACAGGGGGGGAACACTGAAGCTTTCTTT |
| 507 | 151-TL722-TIL-CD8+_CD103+ | TIL | CD8+_CD103+ | TGTGCCAGCAGCCAAGTAGCCAATGAGCAGTTCTTC |
| 508 | 151-TL722-TIL-CD8+_CD103+ | TIL | CD8+_CD103+ | TGTGCCAGCAGCCAAGATCAGGGGCTCCACTTCAGGGAGACCCAGTACTTC |
| 509 | 151-TL722-TIL-CD8+_CD103+ | TIL | CD8+_CD103+ | TGCGCCAGCATCGCTAGCGGGGCTAGCACAGATACGCAGTATTTT |
| 510 | 151-TL722-TIL-CD8+_CD103+ | TIL | CD8+_CD103+ | TGCAGTGCTAGTCCGCCAGATAACTATGGCTACACCTTC |
| 511 | 151-TL722-TIL-CD8+_CD103+ | TIL | CD8+_CD103+ | TGTGCCAGCAGCCTTTCGGACGATCTTCCCGAAAAACTGTTTTTT |
| 512 | 151-TL722-TIL-CD8+_CD103+ | TIL | CD8+_CD103+ | TGCGCCAGCAGAGAACTAGCGGGGGGACTCACGGATACGCAGTATTTT |
| 513 | 151-TL722-TIL-CD8+_CD103+ | TIL | CD8+_CD103+ | TGCAGTGCTCCGCTCATCTCCTGGAACATTCAGTACTTC |
| 514 | 151-TL722-TIL-CD8+_CD103+ | TIL | CD8+_CD103+ | TGTGCCAGCAGGGGACAGCCCTCTGGAAACACCATATATTTT |
| 515 | 151-TL722-TIL-CD8+_CD103+ | TIL | CD8+_CD103+ | TGTGCCAGCAGCTTGGACCAGAACCCTAACTATGGCTACACCTTC |
| 516 | 151-TL722-TIL-CD8+_CD103+ | TIL | CD8+_CD103+ | TGTGCCAGCAGCCAAGATTCAGGGATGAACAATGAGCAGTTCTTC |
| 517 | 151-TL722-TIL-CD8+_CD103+ | TIL | CD8+_CD103+ | TGTGCCAGCAGCCCGGTAACGGTCCGTAATGAAAAACTGTTTTTT |
| 518 | 151-TL722-TIL-CD8+_CD103+ | TIL | CD8+_CD103+ | TGTGCCAGCAGCGTAGCCGGGACAGACAATGAGCAGTTCTTC |
| 519 | 151-TL722-TIL-CD8+_CD103+ | TIL | CD8+_CD103+ | TGTGCCACCAGTGATTCGGACAGGCCTCACAATGAGCAGTTCTTC |
| 520 | 151-TL722-TIL-CD8+_CD103+ | TIL | CD8+_CD103+ | TGTGCCAGCAGTTGGGGAACTGAAGCTTTCTTT |
| 521 | 151-TL722-TIL-CD8+_CD103+ | TIL | CD8+_CD103+ | TGCAGCGTAACCGGGACAGCAAATTCTAACTATGGCTACACCTTC |
| 522 | 151-TL722-TIL-CD8+_CD103+ | TIL | CD8+_CD103+ | TGTGCCAGCAGCGTAGGGAGTGATGGGGTCTATGGCTACACCTTC |
| 523 | 151-TL722-TIL-CD8+_CD103+ | TIL | CD8+_CD103+ | TGTGCCAGCAGTTACTCGGGCGAGTACGAGCAGTACTTC |
| 524 | 151-TL722-TIL-CD8+_CD103+ | TIL | CD8+_CD103+ | TGCAGTGCTAGAGATTGGAAGTTGGATCTCTACGAGCAGTACTTC |
| 525 | 151-TL722-TIL-CD8+_CD103+ | TIL | CD8+_CD103+ | TGTGCCTGGGGGACAGGGGACACTGAAGCTTTCTTT |
| 526 | 151-TL722-TIL-CD8+_CD103+ | TIL | CD8+_CD103+ | TGTGCCAGCAGTTTTGCCGGGCCCCCTTACTTC |
| 527 | 151-TL722-TIL-CD8+_CD103+ | TIL | CD8+_CD103+ | TGTGCCAGCAGCAGGGACTCTGGAAACACCATATATTTT |
| 528 | 151-TL722-TIL-CD8+_CD103+ | TIL | CD8+_CD103+ | TGTGCCAGCAGCTTAGCCGTGCGGCTAGCGGGGGCTGAGCAGTTCTTC |
| 529 | 151-TL722-TIL-CD8+_CD103+ | TIL | CD8+_CD103+ | TGTGCCAGCAGCAAAGGTTTCAGCCGCACCACTTATAATTCACCCCTC |
| | | | | CACTTT |
| 530 | 151-TL722-TIL-CD8+_CD103+ | TIL | CD8+_CD103+ | TGTGCCAGCAGTTGGGGGGCAGGCTCCAGCTATGGCTACACCTTC |
| 531 | 151-TL722-TIL-CD8+_CD103+ | TIL | CD8+_CD103+ | TGTGCCAGCAGCCCAGACCTGAACACTGAAGCTTTCTTT |
| 532 | 151-TL722-TIL-CD8+_CD103+ | TIL | CD8+_CD103+ | TGTGCCATCAGTGAGTCTGGGGGGACAGGTTCTCCCTACGAGCAGTACTTC |
| 533 | 151-TL722-TIL-CD8+_CD103+ | TIL | CD8+_CD103+ | TGTGCCAGCAGCCAAACCGTCCTCCGGGCACCCACGGATGGCTACACCTTC |
| 534 | 151-TL722-TIL-CD8+_CD103+ | TIL | CD8+_CD103+ | TGTGCCAGCAGTTACCAGGGGACAGGAGGATTAAGAGACGAGCAGTACTTC |
| 535 | 151-TL722-TIL-CD8+_CD103+ | TIL | CD8+_CD103+ | TGTGCCAGCAGTTACGCCTCGGGTCCTGTGCATTTT |
| 536 | 151-TL722-TIL-CD8+_CD103+ | TIL | CD8+_CD103+ | TGTGCCACCAGCAGATCCCCGCCCCTGGGTAGCGGAGCCCAAG |
| | | | | AGACCCAGTACTTC |
| 537 | 151-TL722-TIL-CD8+_CD103+ | TIL | CD8+_CD103+ | TGTGCCAGCAGCTTAGGACAGTTATCTGGAAACACCATATATTTT |
| 538 | 151-TL722-TIL-CD8+_CD103+ | TIL | CD8+_CD103+ | TGTGCCAGCAGTTCCGGACAGCGTCGGCCCCAGCATTTT |
| 539 | 151-TL722-TIL-CD8+_CD103+ | TIL | CD8+_CD103+ | TGTGCCAGCACGGGACGGAGCACCGGGGAGCTGTTTTTT |
| 540 | 151-TL722-TIL-CD8+_CD103+ | TIL | CD8+_CD103+ | TGCAGTGCTAGAGACGGGGGGGCAGACGTTCGGTACCAA |
| | | | | GAGACCCAGTACTTC |
| 541 | 151-TL722-TIL-CD8+_CD103+ | TIL | CD8+_CD103+ | TGTGCCAGCAGCTTGACTAGCGGGCCCTACAATGAGCAGTTCTTC |
| 542 | 151-TL722-TIL-CD8+_CD103+ | TIL | CD8+_CD103+ | TGTGCCAGCAGCCCCAGTCGACTGGCGGATACGCAGTATTTT |
| 543 | 151-TL722-TIL-CD8+_CD103+ | TIL | CD8+_CD103+ | TGTGCCAGCAGCCCGGGGCCAGCGGGGGACTCCGAGCAGTTCTTC |
| 544 | 151-TL722-TIL-CD8+_CD103+ | TIL | CD8+_CD103+ | TGCAGTGCTAGCCCCCCGGATCTTTATGGCTACACCTTC |
| 545 | 151-TL722-TIL-CD8+_CD103+ | TIL | CD8+_CD103+ | TGTGCCAGCAGCGAGGGGACAGGCTATCAGCCCCAGCATTTT |
| 546 | 151-TL722-TIL-CD8+_CD103+ | TIL | CD8+_CD103+ | TGCAGTGCTAACGAGTACTTTAGAAATCAGCCCCAGCATTTT |
| 547 | 151-TL722-TIL-CD8+_CD103+ | TIL | CD8+_CD103+ | TGTGCCAGCACCGAGACTAGCGGGGGATACAATGAGCAGTTCTTC |
| 548 | 151-TL722-TIL-CD8+_CD103+ | TIL | CD8+_CD103+ | TGCGCCAGCAGCCAAGATGGGGTGAGACTAAACATTCAGTACTTC |
| 549 | 151-TL722-TIL-CD8+_CD103+ | TIL | CD8+_CD103+ | TGTGCCAGCAGCTTAGAACTAGCGGGGTTCTCCAATGAGCAGTTCTTC |
| 550 | 151-TL722-TIL-CD8+_CD103+ | TIL | CD8+_CD103+ | TGTGCCAGCAGCCCCTCCGGGATTGGCTACACCTTC |
| 551 | 151-TL722-TIL-CD8+_CD103+ | TIL | CD8+_CD103+ | TGTGCCAGCAGCGCCCTGTCCGAGCGAAGTACTATGGCTACACCTTC |
| 552 | 151-TL722-TIL-CD8+_CD103+ | TIL | CD8+_CD103+ | TGTGCCAGCAGGACTAGCAACACCGGGGAGCTGTTTTTT |
| 553 | 151-TL722-TIL-CD8+_CD103+ | TIL | CD8+_CD103+ | TGCGCCAGCAGCCTGAGGGAAGAATTGAACACTGAAGCTTTCTTT |
| 554 | 151-TL722-TIL-CD8+_CD103+ | TIL | CD8+_CD103+ | TGTGCCAGCAGCTCCTCGGACAGGACTGGCAATGAGCAGTTCTTC |
| 555 | 151-TL722-TIL-CD8+_CD103+ | TIL | CD8+_CD103+ | TGCGCCAGCGGGACAGGGGATGCGACCCAGTACTTC |
| 556 | 151-TL722-TIL-CD8+_CD103+ | TIL | CD8+_CD103+ | TGTGCCAGCAGTTTCACCCCCAACACCGGGGAGCTGTTTTTT |
| 557 | 151-TL722-TIL-CD8+_CD103+ | TIL | CD8+_CD103+ | TGTGCCAGCAGTCCTACAGCAAACAATCAGCCCCAGCATTTT |
| 558 | 151-TL722-TIL-CD8+_CD103+ | TIL | CD8+_CD103+ | TGTGCCAGCAGCTTAAGAATCGGGGGGTCACTTTCACCAATC |
| | | | | CAGATCTACGAGCAGTACTTC |
| 559 | 151-TL722-TIL-CD8+_CD103+ | TIL | CD8+_CD103+ | TGTGCCAGCAGCCCGATGGGGGACACCGGGGAGCTGTTTTTT |
| 560 | 151-TL722-TIL-CD8+_CD103+ | TIL | CD8+_CD103+ | TGTGCCAGCAGTTTGCGCCCGGGACTAGCGGGGGGCCCAGAT |
| | | | | ACGCAGTATTTT |
| 561 | 151-TL722-TIL-CD8+_CD103+ | TIL | CD8+_CD103+ | TGTGCCAGTAGTATAGGGACGTACGCGAACACCGGGGAGCTGTTTTTT |
| 562 | 151-TL722-TIL-CD8+_CD103+ | TIL | CD8+_CD103+ | TGTGCCAGCAGCCTGATCCCCGGGGCCACAGATACGCAGTATTTT |
| 563 | 151-TL722-TIL-CD8+_CD103+ | TIL | CD8+_CD103+ | TGTGCCAGCAGCCAAGGGACTAGCGGGGGGGCAGATACGCAGTATTTT |
| 564 | 151-TL722-TIL-CD8+_CD103+ | TIL | CD8+_CD103+ | TGTGCCAGCAGCCTGATGGCAGATACGCAGTATTTT |
| 565 | 151-TL722-TIL-CD8+_CD103+ | TIL | CD8+_CD103+ | TGTGCCAGCAGCCAGCGTTGGGGGAGCACAGATACGCAGTATTTT |
| 566 | 151-TL722-TIL-CD8+_CD103+ | TIL | CD8+_CD103+ | TGTGCCAGCAGCTTGTTCGAGGGGGCCCAAGAGACCCAGTACTTC |
| 567 | 151-TL722-TIL-CD8+_CD103+ | TIL | CD8+_CD103+ | TGTGCCAGCAGTTTGGGGGGACAGAACTATGGCTACACCTTC |
| 568 | 151-TL722-TIL-CD8+_CD103+ | TIL | CD8+_CD103+ | TGTGCCAGCAGCGTTGGGGACACGAACACTGAAGCTTTCTTT |
| 569 | 151-TL722-TIL-CD8+_CD103+ | TIL | CD8+_CD103+ | TGTGCCAGCAGCCCATCGGGGGGCACTGAAGCTTTCTTT |
| 570 | 151-TL722-TIL-CD8+_CD103+ | TIL | CD8+_CD103+ | TGTGCCAGCAAAAACCACGGGACAGGCTTAGACGAGCAGTACTTC |
| 571 | 151-TL722-TIL-CD8+_CD103+ | TIL | CD8+_CD103+ | TGTGCCAGCAGTACTGGACAGGCAGAAAAACTGTTTTTT |
| 572 | 151-TL722-TIL-CD8+_CD103+ | TIL | CD8+_CD103+ | TGTGCCAGCAGCGTAGCCCCCCAGGGGGCGAGTTCTGGCTACACCTTC |
| 573 | 151-TL722-TIL-CD8+_CD103+ | TIL | CD8+_CD103+ | TGTGCCAGCGGGACGGGATCCTACGAGCAGTACTTC |
| 574 | 151-TL722-TIL-CD8+_CD103+ | TIL | CD8+_CD103+ | TGCAGCGTCCTTCCCGACAGTCCCCAACGGGCCCAGTACTTC |
| 575 | 151-TL722-TIL-CD8+_CD103+ | TIL | CD8+_CD103+ | TGTGCCAGCAGCGTAGCGGGCGGGGGCTCCTACGAGCAGTACTTC |
| 576 | 151-TL722-TIL-CD8+_CD103+ | TIL | CD8+_CD103+ | TGTGCCAGCAGGCGTACCTCTGTGAACGTCCTGACTTTC |
| 577 | 151-TL722-TIL-CD8+_CD103+ | TIL | CD8+_CD103+ | TGCGCCAGCAGCCAACTAGCGGGGGGGCGCGATGAGCAGTTCTTC |
| 578 | 151-TL722-TIL-CD8+_CD103+ | TIL | CD8+_CD103+ | TGTGCCAGCAGTTACTCGAATGGGGGGTGGAACTATGGCTACACCTTC |
| 579 | 151-TL722-TIL-CD8+_CD103+ | TIL | CD8+_CD103+ | TGTGCCAGCAGTCGGGACAGGGGTATCACAGATACGCAGTATTTT |
| 580 | 151-TL722-TIL-CD8+_CD103+ | TIL | CD8+_CD103+ | TGCAGTGCTAGAGATTGGAGTGGCATCCCCGGGGAGCTGTTTTTT |
| 581 | 151-TL722-TIL-CD8+_CD103+ | TIL | CD8+_CD103+ | TGTGCCAGCAGTTACTCCTGGGGTTCTGAAGCTTTCTTT |
| 582 | 151-TL722-TIL-CD8+_CD103+ | TIL | CD8+_CD103+ | TGCGCCAGCAGCTTGCTCGGGGTTCACGGCCTGAACACTGAAGCTTTCTTT |
| 583 | 151-TL722-TIL-CD8+_CD103+ | TIL | CD8+_CD103+ | TGCAGTGCTACGGCTCCCGGGACTACCTCCTACGAGCAGTACTTC |
| 584 | 151-TL722-TIL-CD8+_CD103+ | TIL | CD8+_CD103+ | TGTGCCAGCTCACCTTACCCCTTGGGAACAGATACGCAGTATTTT |
| 585 | 151-TL722-TIL-CD8+_CD103+ | TIL | CD8+_CD103+ | TGTGCCAGCAGCCTGGGACAGGGGAACACTGAAGCTTTCTTT |
| 586 | 151-TL722-TIL-CD8+_CD103+ | TIL | CD8+_CD103+ | TGTGCCAGCAGTCGCCGGATTGACCTTGGCGAAGAGACCCAGTACTTC |
| 587 | 151-TL722-TIL-CD8+_CD103+ | TIL | CD8+_CD103+ | TGTGCCAGCAGTTCTCAGGGTCCCCAATTGGCCCAGCATTTT |
| 588 | 151-TL722-TIL-CD8+_CD103+ | TIL | CD8+_CD103+ | TGTGCCAGCAGCCCACTCCGGGACAGGGTTCCCCTGGCCTTC |
| 589 | 151-TL722-TIL-CD8+_CD103+ | TIL | CD8+_CD103+ | TGTGCCAGCAGCTTTGGCGGGGGGACCTACGAGCAGTACTTC |
| 590 | 151-TL722-TIL-CD8+_CD103+ | TIL | CD8+_CD103+ | TGTGCCAGCAGCTCCCTCGGGGGTACCAATCTTGCAGATACGCAGTATTTT |
| 591 | 151-TL722-TIL-CD8+_CD103+ | TIL | CD8+_CD103+ | TGTGCCAGCAGTTTTGGTCCGTTTGGCACCGGGGAGCTGTTTTTT |
| 592 | 151-TL722-TIL-CD8+_CD103+ | TIL | CD8+_CD103+ | TGTGCCAGCAGTTTACAGGGGGCGCTAGAAGGCTACACCTTC |
| 593 | 151-TL722-TIL-CD8+_CD103+ | TIL | CD8+_CD103+ | TGTGCCAGCAGCAAGACTAGCGGGGGGTATAGCACAGATACGCAGTATTTT |
| 594 | 151-TL722-TIL-CD8+_CD103+ | TIL | CD8+_CD103+ | TGTGCCAGCAGCATTACAGGGGGGGATCAGCCCCAGCATTTT |
| 595 | 151-TL722-TIL-CD8+_CD103+ | TIL | CD8+_CD103+ | TGTGCCAGCAGTCTGGGGCTTCAAGAGACCCAGTACTTC |
| 596 | 151-TL722-TIL-CD8+_CD103+ | TIL | CD8+_CD103+ | TGTGCCGGAACGACAGGGTACGGGAAGAGCAATCAGCCCCAGCATTTT |
| 597 | 151-TL722-TIL-CD8+_CD103+ | TIL | CD8+_CD103+ | TGTGCCAGCAGCTTATACGGGGCGGGTGAGCAGTACTTC |
| 598 | 151-TL722-TIL-CD8+_CD103+ | TIL | CD8+_CD103+ | TGCGCCAGCAGCTCCCCGGTCAGGCCAGATACGCAGTATTTT |
| 599 | 151-TL722-TIL-CD8+_CD103+ | TIL | CD8+_CD103+ | TGTGCCAGCAGCCAAGTAGCCAATGAGCAGTTATTC |
| 600 | 151-TL722-TIL-CD8+_CD103+ | TIL | CD8+_CD103+ | TGTGCCAGCAGTGCAGGGGGATCCTACAATGAGCAGTTCTTC |
| 601 | 151-TL722-TIL-CD8+_CD103+ | TIL | CD8+_CD103+ | TGTGCCAGTAGTATTGGACAGGGGTACGAGCAGTACTTC |
| 602 | 151-TL722-TIL-CD8+_CD103+ | TIL | CD8+_CD103+ | TGCGCCAGCAGCCACCAAGCCGATGAGCAGTTCTTC |
| 603 | 151-TL722-TIL-CD8+_CD103+ | TIL | CD8+_CD103+ | TGTGCCAGCAGTCCCTTAGGTGTAAGCGGGGCAAGCTCCTAC |
| | | | | AATGAGCAGTTCTTC |
| 604 | 151-TL722-TIL-CD8+_CD103+ | TIL | CD8+_CD103+ | TGTGCCAGCAGTGGTACAGGGGCGTCTAATGAAAAACTGTTTTTT |
| 605 | 151-TL722-TIL-CD8+_CD103+ | TIL | CD8+_CD103+ | TGTGCCAGCAGTCCAGCGACTAGCGGGGGCCGGGACGAGCAGTACTTC |
| 606 | 151-TL722-TIL-CD8+_CD103+ | TIL | CD8+_CD103+ | TGTGCCAGCAGCTTATTTGAGGACAGGGGGCTAAGAGAGACCCAGTACTTC |
| 607 | 151-TL722-TIL-CD8+_CD103+ | TIL | CD8+_CD103+ | TGTGCCAGCAGCTCTGGACAGACAGATACGCAGTATTTT |
| 608 | 151-TL722-TIL-CD8+_CD103+ | TIL | CD8+_CD103+ | TGTGCCAGCAGTCAAAACGCCTACAGGGGGAAGCCCCAGCATTTT |
| 609 | 151-TL722-TIL-CD8+_CD103+ | TIL | CD8+_CD103+ | TGTGCCAGCAGCCTGTCCGGGGGGGGAATGGGTGAGCAGTTCTTC |
| 610 | 151-TL722-TIL-CD8+_CD103+ | TIL | CD8+_CD103+ | TGTGCCAGCAGTTCCGTAGGGGCTAGAGAGCAGTACTTC |
| 611 | 151-TL722-TIL-CD8+_CD103+ | TIL | CD8+_CD103+ | TGTGCCAGCGAACTAGCGTCCGGGGAGCTGTTTTTT |
| 612 | 151-TL722-TIL-CD8+_CD103+ | TIL | CD8+_CD103+ | TGTGCCAGCAGGGACAGGGGGGGTCAGCCCCAGCATTTT |
| 613 | 151-TL722-TIL-CD8+_CD103+ | TIL | CD8+_CD103+ | TGTGCCAGTAGAGATGCAGGGGCGGGAGGCTACACCTTC |
| 614 | 151-TL722-TIL-CD8+_CD103+ | TIL | CD8+_CD103+ | TGTGCCAGCCCAACGGGGGCCTATGGCTACACCTTC |
| 615 | 151-TL722-TIL-CD8+_CD103+ | TIL | CD8+_CD103+ | TGCGCCAGCAGCCGGACTAGCGGGGGGGCGGATGAGCAGTTCTTC |
| 616 | 152-TL722-TIL-CD8+_CD103− | TIL | CD8+_CD103− | TGTGCCAGCACCGGGGGAGACCGCTACTATGGCTACACCTTC |
| 617 | 152-TL722-TIL-CD8+_CD103− | TIL | CD8+_CD103− | TGTGCCATCAGTGACGACCGGGACAGGGATGGATTCCGATACA |
| | | | | ATCAGCCCCAGCATTTT |
| 618 | 152-TL722-TIL-CD8+_CD103− | TIL | CD8+_CD103− | TGTGCCACCAGTGATTCGGACAGGCCTCACAATGAGCAGTTCTTC |
| 619 | 152-TL722-TIL-CD8+_CD103− | TIL | CD8+_CD103− | TGTGCCAGCAGCAAAGGTTTCAGCCGCACCACTTATAAT |
| | | | | TCACCCCTCCACTTT |
| 620 | 152-TL722-TIL-CD8+_CD103− | TIL | CD8+_CD103− | TGTGCCACCGGGTATAGTAGCAATCAGCCCCAGCATTTT |
| 621 | 152-TL722-TIL-CD8+_CD103− | TIL | CD8+_CD103− | TGTGCCAGCAGTTACTCGGGCGAGTACGAGCAGTACTTC |
| 622 | 152-TL722-TIL-CD8+_CD103− | TIL | CD8+_CD103− | TGTGCCAGCAGCGAGGGGCAGGAGACAGATACGCAGTATTTT |
| 623 | 152-TL722-TIL-CD8+_CD103− | TIL | CD8+_CD103− | TGCAGTGCTAGGGGTTGGGACAGGGGAACCTACGAGCAGTACTTC |
| 624 | 152-TL722-TIL-CD8+_CD103− | TIL | CD8+_CD103− | TGTGCCAGCAGTTTTGCCGGGCCCCCTTACTTC |
| 625 | 152-TL722-TIL-CD8+_CD103− | TIL | CD8+_CD103− | TGTGCCAGCAGCTTAGGGACAGGATCTAACTATGGCTACACCTTC |
| 626 | 152-TL722-TIL-CD8+_CD103− | TIL | CD8+_CD103− | TGTGCCAGTAGTATAGACGGCACTTCCTACGAGCAGTACTTC |
| 627 | 152-TL722-TIL-CD8+_CD103− | TIL | CD8+_CD103− | TGCAGCGTGGGACAGGGGGGCACTAATGAAAAACTGTTTTTT |
| 628 | 152-TL722-TIL-CD8+_CD103− | TIL | CD8+_CD103− | TGTGCCAGCAGTGGACGGTTAGCGGACACCGGGGAGCTGTTTTTT |
| 629 | 152-TL722-TIL-CD8+_CD103− | TIL | CD8+_CD103− | TGTGCCAGCAGCTTAGGACGGGACGGCTACACCTTC |
| 630 | 152-TL722-TIL-CD8+_CD103− | TIL | CD8+_CD103− | TGTGCCAGCAGCTTCCGGGACAGCACCTACGAGCAGTACTTC |
| 631 | 152-TL722-TIL-CD8+_CD103− | TIL | CD8+_CD103− | TGCAGTGCTAGAATAATCCTTGCCTACGAGCAGTACTTC |
| 632 | 152-TL722-TIL-CD8+_CD103− | TIL | CD8+_CD103− | TGTGCCAGCAGTGAAGGGGACACGCAGTACTTC |
| 633 | 152-TL722-TIL-CD8+_CD103− | TIL | CD8+_CD103− | TGCGCCAGCAGTGAGGACGGTATGAACACTGAAGCTTTCTTT |
| 634 | 152-TL722-TIL-CD8+_CD103− | TIL | CD8+_CD103− | TGTGCCAGCAGTTATACGGCGAAGATCCTGACTTTC |
| 635 | 152-TL722-TIL-CD8+_CD103− | TIL | CD8+_CD103− | TGTGCCAGCAGCTTTAGAGCAGGGGAAACCAACTATGGCTACACCTTC |
| 636 | 152-TL722-TIL-CD8+_CD103− | TIL | CD8+_CD103− | TGTGCCTGGAGTGGGACAGGCGCCTACGAGCAGTACTTC |
| 637 | 152-TL722-TIL-CD8+_CD103− | TIL | CD8+_CD103− | TGTGCCAGCAGACGGCTAGCGGGCACCGGGGAGCTGTTTTTT |
| 638 | 152-TL722-TIL-CD8+_CD103− | TIL | CD8+_CD103− | TGTGCCAGCAGTCCAGCCCCAGGAGGCCAGCCCCAGCATTTT |
| 639 | 152-TL722-TIL-CD8+_CD103− | TIL | CD8+_CD103− | TGCAGCGTAACCGGGACAGCAAATTCTAACTATGGCTACACCTTC |
| 640 | 152-TL722-TIL-CD8+_CD103− | TIL | CD8+_CD103− | TGTGCCACCAGCAGAGGAAGGGGAGTAGAGACCCAGTACTTC |
| 641 | 152-TL722-TIL-CD8+_CD103− | TIL | CD8+_CD103− | TGTGCCAGCAGGCAAGAGTCGGGGCAGCCCCAGCATTTT |
| 642 | 152-TL722-TIL-CD8+_CD103− | TIL | CD8+_CD103− | TGTGCCAGCAGTTACTCGACAGGGGTTCCTGAAAAACTGTTTTTT |
| 643 | 152-TL722-TIL-CD8+_CD103− | TIL | CD8+_CD103− | TGTGCCAGCAGTTTTGGTCCGTTTGGCACCGGGGAGCTGTTTTTT |
| 644 | 152-TL722-TIL-CD8+_CD103− | TIL | CD8+_CD103− | TGTGCCAGCGATCCGGGACAGGGGAATGAGCAGTTCTTC |
| 645 | 152-TL722-TIL-CD8+_CD103− | TIL | CD8+_CD103− | TGTGCCAGCAGCTCTTGGGACAGGATAAAGGAGCAGTACTTC |
| 646 | 152-TL722-TIL-CD8+_CD103− | TIL | CD8+_CD103− | TGTGCCAGCAGCCCCGGGGGAGGGGGCTGGTACAATGAGCAGTTCTTC |
| 647 | 152-TL722-TIL-CD8+_CD103− | TIL | CD8+_CD103− | TGTGCCAGCAGTCCCGGGACAGGGGGCAGGCCCCAGCATTTT |
| 648 | 152-TL722-TIL-CD8+_CD103− | TIL | CD8+_CD103− | TGTGCCAGCAGTGATGGACAGGCCAACGTCCTGACTTTC |
| 649 | 152-TL722-TIL-CD8+_CD103− | TIL | CD8+_CD103− | TGTGCCAGCAGTTTTGTACAGGGGACCGATACGCAGTATTTT |
| 650 | 152-TL722-TIL-CD8+_CD103− | TIL | CD8+_CD103− | TGTGCCAGCAGGAGACAGGGAGACTACGAGCAGTACTTC |
| 651 | 152-TL722-TIL-CD8+_CD103− | TIL | CD8+_CD103− | TGCAGCGTTGGGAGCAGCGGAACTAATGAAAAACTGTTTTTT |
| 652 | 152-TL722-TIL-CD8+_CD103− | TIL | CD8+_CD103− | TGTGCCAGCAGCTTGACAGGTGGGGACAATCAGCCCCAGCATTTT |
| 653 | 152-TL722-TIL-CD8+_CD103− | TIL | CD8+_CD103− | TGTGCCAGCAGCAGTAGCGGGTACCAAGAGACCCAGTACTTC |
| 654 | 152-TL722-TIL-CD8+_CD103− | TIL | CD8+_CD103− | TGTGCCAGCAGTCTCTTCCTTTGGACGGGGAGCTCCTACAAT |
| | | | | GAGCAGTTCTTC |
| 655 | 152-TL722-TIL-CD8+_CD103− | TIL | CD8+_CD103− | TGCGCCAGCAGCTTGGGGAATGAAGCTTTCTTT |
| 656 | 152-TL722-TIL-CD8+_CD103− | TIL | CD8+_CD103− | TGTGCGACGACCCGTTCCGACTCCTACGAGCAGTACTTC |
| 657 | 152-TL722-TIL-CD8+_CD103− | TIL | CD8+_CD103− | TGTGCCAGCTCCCTGCCCGGGCGGGGGCGCGAGCAGTACTTC |
| 658 | 152-TL722-TIL-CD8+_CD103− | TIL | CD8+_CD103− | TGTGCCAGCAGCCCAGACAGGGGCCGAGAGACCCAGTACTTC |
| 659 | 152-TL722-TIL-CD8+_CD103− | TIL | CD8+_CD103− | TGTGCCAGCAGCTTGGGGGGCGGCTCCTACGAGCAGTACTTC |
| 660 | 152-TL722-TIL-CD8+_CD103− | TIL | CD8+_CD103− | TGTGCCAGCAGTTTAAGTCCGATCACCGGGGAGCTGTTTTTT |
| 661 | 152-TL722-TIL-CD8+_CD103− | TIL | CD8+_CD103− | TGTGCCAGTAGTATAGATAATACAGGGCGCAATCAGCCCCAGCATTTT |
| 662 | 152-TL722-TIL-CD8+_CD103− | TIL | CD8+_CD103− | TGTGCCAGCAGCTTCGGCCCGGGGGTCCCACCGTACGATACGCAGTATTTT |
| 663 | 152-TL722-TIL-CD8+_CD103− | TIL | CD8+_CD103− | TGTGCCAGCAGCCAGCTCAAATCAATGGGCTATGGCTACACCTTC |
| 664 | 152-TL722-TIL-CD8+_CD103− | TIL | CD8+_CD103− | TGTGCCACCAGTGAATCTACCCTTTCTGGCACGGACACAGA |
| | | | | TACGCAGTATTTT |
| 665 | 152-TL722-TIL-CD8+_CD103− | TIL | CD8+_CD103− | TGCGCCAGCAGCTTGGCGTCGCTTAGCACAGATACGCAGTATTTT |
| 666 | 152-TL722-TIL-CD8+_CD103− | TIL | CD8+_CD103− | TGTGCCAGCAGCTCCCGGACGGGCTCCTACGAGCAGTACTTC |
| 667 | 152-TL722-TIL-CD8+_CD103− | TIL | CD8+_CD103− | TGTGCCAGCAGCTCCGGGAACAGCCGGCTTACGCAGTATTTT |
| 668 | 152-TL722-TIL-CD8+_CD103− | TIL | CD8+_CD103− | TGTGCCAGCACCCAGACAGGGAACAATCAGCCCCAGCATTTT |
| 669 | 152-TL722-TIL-CD8+_CD103− | TIL | CD8+_CD103− | TGTGCCAGCAGCACATCGACAGGGATGGCTGAAAAACTGTTTTTT |
| 670 | 152-TL722-TIL-CD8+_CD103− | TIL | CD8+_CD103− | TGCGCCAGCAGTCCCGGACAGGGGGCGACAGATACGCAGTATTTT |
| 671 | 152-TL722-TIL-CD8+_CD103− | TIL | CD8+_CD103− | TGTGCCAGTAGTATAGATCTATATAGCAATCAGCCCCAGCATTTT |
| 672 | 152-TL722-TIL-CD8+_CD103− | TIL | CD8+_CD103− | TGTGCCAGCAGGGGGTTTGCCAAAAACATTCAGTACTTC |
| 673 | 152-TL722-TIL-CD8+_CD103− | TIL | CD8+_CD103− | TGCGCCAGCAGCCAAGGGACAGGGAACTACGAGCAGTACTTC |
| 674 | 152-TL722-TIL-CD8+_CD103− | TIL | CD8+_CD103− | TGTGCCAGCAGCTTTCTCCCGGGACAGGGGAACACCGGGGAGCTGTTTTTT |
| 675 | 152-TL722-TIL-CD8+_CD103− | TIL | CD8+_CD103− | TGTGCCAGCAGTGAATGGGCTAGCGGGGAGACCCAGTACTTC |
| 676 | 157-TL704-TIL-CD8+_CD103+ | TIL | CD8+_CD103+ | TGCGCCAGCAGCCAAGATCTCGGTAAGCAGCCCCAGCATTTT |
| 677 | 157-TL704-TIL-CD8+_CD103+ | TIL | CD8+_CD103+ | TGTGCCAGCAGTTTATCGTTGACCTACGGTAGAGGGCAGCCCCAGCATTTT |
| 678 | 157-TL704-TIL-CD8+_CD103+ | TIL | CD8+_CD103+ | TGTGCCAGCAGTACGACCCACCCTAATCAGCCCCAGCATTTT |
| 679 | 157-TL704-TIL-CD8+_CD103+ | TIL | CD8+_CD103+ | TGTGCCAGCAGCGAGGAACAGGGGGCGCTGAACGAGCAGTACTTC |
| 680 | 157-TL704-TIL-CD8+_CD103+ | TIL | CD8+_CD103+ | TGTGCCAGCACGAGGTGGGACAGGGAGCCCGAAGGGTACGAGCAGTACTTC |
| 681 | 157-TL704-TIL-CD8+_CD103+ | TIL | CD8+_CD103+ | TGCGCCAGCAGCTCGGGACACGTCTATGGCTACACCTTC |
| 682 | 157-TL704-TIL-CD8+_CD103+ | TIL | CD8+_CD103+ | TGTGCCAGCAGTTACTTACAGGGGATGAACACTGAAGCTTTCTTT |
| 683 | 157-TL704-TIL-CD8+_CD103+ | TIL | CD8+_CD103+ | TGTGCCAGCAGCTTAGGCTACGAGCAGTACTTC |
| 684 | 157-TL704-TIL-CD8+_CD103+ | TIL | CD8+_CD103+ | TGCAGTGCCCGTCCGTTGGGACAAATCTACGAGCAGTACTTC |
| 685 | 157-TL704-TIL-CD8+_CD103+ | TIL | CD8+_CD103+ | TGTGCCAGCAGTTTTTCGGCCGGGCTCAATCAGCCCCAGCATTTT |
| 686 | 157-TL704-TIL-CD8+_CD103+ | TIL | CD8+_CD103+ | TGTGCCAGTAATGCATATGATAATTCACCCCTCCACTTT |
| 687 | 157-TL704-TIL-CD8+_CD103+ | TIL | CD8+_CD103+ | TGTGCCAGCAGCTTTGGACTAGCGGTCTACGAGCAGTACTTC |
| 688 | 157-TL704-TIL-CD8+_CD103+ | TIL | CD8+_CD103+ | TGTGCCATCAGTGAGCCCGGGACTACCCGGGTCGATGAGCAGTTCTTC |
| 689 | 157-TL704-TIL-CD8+_CD103+ | TIL | CD8+_CD103+ | TGTGCCAGCAGCTCGCCCGGGAGGACGCCAGGAAACACCATATATTTT |
| 690 | 157-TL704-TIL-CD8+_CD103+ | TIL | CD8+_CD103+ | TGTGCCAGCAGTTTCCGGGGACAGGTTCTGAACACCGGGGAGCTGTTTTTT |
| 691 | 157-TL704-TIL-CD8+_CD103+ | TIL | CD8+_CD103+ | TGTGCCAGCTCACCGGTACAAACAGATACGCAGTATTTT |
| 692 | 157-TL704-TIL-CD8+_CD103+ | TIL | CD8+_CD103+ | TGTGCCAGTAGGCCCGCTAGCGGGAGGACAGATACGCAGTATTTT |
| 693 | 157-TL704-TIL-CD8+_CD103+ | TIL | CD8+_CD103+ | TGTGCCAGCAGTGAAAGCTACGAGCAGTACTTC |
| 694 | 157-TL704-TIL-CD8+_CD103+ | TIL | CD8+_CD103+ | TGCGCCAGCAGCCCGGGACTAGCGGAGAGCTCCTACGAGCAGTACTTC |
| 695 | 157-TL704-TIL-CD8+_CD103+ | TIL | CD8+_CD103+ | TGTGCCAGCAGTTTTTCGCGACACTTCTCCTACGAGCAGTACTTC |
| 696 | 157-TL704-TIL-CD8+_CD103+ | TIL | CD8+_CD103+ | TGTGCCAGCAGCTTAGGGGGCAGCTCCTCAGGGGATGGCTACACCTTC |
| 697 | 157-TL704-TIL-CD8+_CD103+ | TIL | CD8+_CD103+ | TGTGCCAGTAGTATAGGGGGAGCTAATGAGCAGTTCTTC |
| 698 | 157-TL704-TIL-CD8+_CD103+ | TIL | CD8+_CD103+ | TGTGCCACCAGCCCCCGGGGGGCGGACAGGACCTATAAC |
| | | | | TATGGCTACACCTTC |
| 699 | 157-TL704-TIL-CD8+_CD103+ | TIL | CD8+_CD103+ | TGTGCCAGCGGACTGGACTACGAGCAGTACTTC |
| 700 | 157-TL704-TIL-CD8+_CD103+ | TIL | CD8+_CD103+ | TGTGCCAGCAGTCCGTACGGGGACGCTTTGGGAGAGACCCAGTACTTC |
| 701 | 157-TL704-TIL-CD8+_CD103+ | TIL | CD8+_CD103+ | TGTGCCAGCAGTTCTGGAGGGGACGGATACGAGCAGTACTTC |
| 702 | 157-TL704-TIL-CD8+_CD103+ | TIL | CD8+_CD103+ | TGTGCCAGCAGCGGAACAGGGCTCTATAATTCACCCCTCCACTTT |
| 703 | 157-TL704-TIL-CD8+_CD103+ | TIL | CD8+_CD103+ | TGTGCCACCAGCATGGTAGCGGGAGGTACCTACGAGCAGTACTTC |
| 704 | 157-TL704-TIL-CD8+_CD103+ | TIL | CD8+_CD103+ | TGTGCCTGGAATTTTGGACAGGGGATATCCTACGAGCAGTACTTC |
| 705 | 157-TL704-TIL-CD8+_CD103+ | TIL | CD8+_CD103+ | TGCGCCAGCAGCCAAGTTCTGAACACTGAAGCTTTCTTT |
| 706 | 157-TL704-TIL-CD8+_CD103+ | TIL | CD8+_CD103+ | TGTGCCAGCAGCCAAGCGGGAGGGATCTCCTACGAGCAGTACTTC |
| 707 | 157-TL704-TIL-CD8+_CD103+ | TIL | CD8+_CD103+ | TGTGCCAGCAGCCAAGATTGGGGGACTAGCGGGGGGTCTTGGAG |
| | | | | TGAGCAGTTCTTC |
| 708 | 157-TL704-TIL-CD8+_CD103+ | TIL | CD8+_CD103+ | TGCAGCGCCTTAATGCGAGAGGCTGAAGCTTTCTTT |
| 709 | 157-TL704-TIL-CD8+_CD103+ | TIL | CD8+_CD103+ | TGTGCCAGCAGCTGGGGGACAGTTAATGAGCAGTTCTTC |
| 710 | 157-TL704-TIL-CD8+_CD103+ | TIL | CD8+_CD103+ | TGTGCCACCAGCAGAGAGCGGACCCCCACAGATACGCAGTATTTT |
| 711 | 157-TL704-TIL-CD8+_CD103+ | TIL | CD8+_CD103+ | TGTGCCAGCAGTTACTCGTACGAGCAGTACTTC |
| 712 | 157-TL704-TIL-CD8+_CD103+ | TIL | CD8+_CD103+ | TGTGCCAGCAGCTTATGGGGGGGCAGTGAAGCTTTCTTT |
| 713 | 157-TL704-TIL-CD8+_CD103+ | TIL | CD8+_CD103+ | TGTGCCAGCAGCTTAGGGGGGGGCACAGATACGCAGTATTTT |
| 714 | 157-TL704-TIL-CD8+_CD103+ | TIL | CD8+_CD103+ | TGTGCCAGCAGCCCCCCCACTAGCGGCCAAGAGACCCAGTACTTC |
| 715 | 157-TL704-TIL-CD8+_CD103+ | TIL | CD8+_CD103+ | TGCGCCAGCAGCCCCTCGGATGGACTGCCGTACCAAGAGACCCAGTACTTC |
| 716 | 157-TL704-TIL-CD8+_CD103+ | TIL | CD8+_CD103+ | TGTGCCAGTAGTGCGGGAAACACTGAAGCTTTCTTT |
| 717 | 157-TL704-TIL-CD8+_CD103+ | TIL | CD8+_CD103+ | TGCAGTGTCCGGGACAGGGCTTCCGGGGGGGAGACCCAGTACTTC |
| 718 | 157-TL704-TIL-CD8+_CD103+ | TIL | CD8+_CD103+ | TGCAGTGCAAGTGCAGGGGTTGAGCAGTTCTTC |
| 719 | 157-TL704-TIL-CD8+_CD103+ | TIL | CD8+_CD103+ | TGCGCCAGCAGCCACGAAGGGACCGATTCCACCTACTATAATTCACCC |
| | | | | CTCCACTTT |
| 720 | 157-TL704-TIL-CD8+_CD103+ | TIL | CD8+_CD103+ | TGTGCCAGCGGACAGGGGAGAGCAGATACGCAGTATTTT |
| 721 | 157-TL704-TIL-CD8+_CD103+ | TIL | CD8+_CD103+ | TGCGCCAGCAGCTTGGATGTCGGGAACATTCAGTACTTC |
| 722 | 157-TL704-TIL-CD8+_CD103+ | TIL | CD8+_CD103+ | TGTGCCAGCAGTCCGCCCTCAGGGGGCGGGGAGACCCAGTACTTC |
| 723 | 157-TL704-TIL-CD8+_CD103+ | TIL | CD8+_CD103+ | TGTGCCAGTACCCCGACAGGGTTAAACACTGAAGCTTTCTTT |
| 724 | 157-TL704-TIL-CD8+_CD103+ | TIL | CD8+_CD103+ | TGCGCCAGCAGCCAAGATAGGGGGACAACCGGGATGAACACT |
| | | | | GAAGCTTTCTTT |
| 725 | 157-TL704-TIL-CD8+_CD103+ | TIL | CD8+_CD103+ | TGTGCCAGCAGTTTAGCGTCGCAGGAACAAGAGACCCAGTACTTC |
| 726 | 157-TL704-TIL-CD8+_CD103+ | TIL | CD8+_CD103+ | TGTGCCACCAGTGCATTATCCACAGATACGCAGTATTTT |
| 727 | 157-TL704-TIL-CD8+_CD103+ | TIL | CD8+_CD103+ | TGTGCCAGCAGCCAAGATTGGGGCGGAGGCTCCTACAATGAGCAGTTCTTC |
| 728 | 157-TL704-TIL-CD8+_CD103+ | TIL | CD8+_CD103+ | TGTGCCAGCATGGGTGGATACGAGCAGTACTTC |
| 729 | 157-TL704-TIL-CD8+_CD103+ | TIL | CD8+_CD103+ | TGTGCCACCAGCAGTGGGACTAGCGGGGGTTACGAGCAGTACTTC |
| 730 | 157-TL704-TIL-CD8+_CD103+ | TIL | CD8+_CD103+ | TGCAGTGCTTCAGGACTAGCTGGGATCTACGAGCAGTACTTC |
| 731 | 157-TL704-TIL-CD8+_CD103+ | TIL | CD8+_CD103+ | TGTGCCAGCAGCCCTTCGGCTAGCGGGCTAAATGAGCAGTTCTTC |
| 732 | 157-TL704-TIL-CD8+_CD103+ | TIL | CD8+_CD103+ | TGTGCCAGCAGTCTCAACCCGGGACTAGCGGGAGAGACCCAGTACTTC |
| 733 | 157-TL704-TIL-CD8+_CD103+ | TIL | CD8+_CD103+ | TGTGCCAGCTCTCAGGGGAGGAACGAGCAGTACTTC |
| 734 | 157-TL704-TIL-CD8+_CD103+ | TIL | CD8+_CD103+ | TGTGCCAGCAGCGAGGAACAGGGGGCGCTGAACGAGCAGTACTTC |
| 735 | 157-TL704-TIL-CD8+_CD103+ | TIL | CD8+_CD103+ | TGCAGCGTTCTTCCGAGGGCGGGAGAAATCTACGAGCAGTACTTC |
| 736 | 157-TL704-TIL-CD8+_CD103+ | TIL | CD8+_CD103+ | TGTGCCAGCAGCTTAGTTCACAGCCTCTCCTACGAGCAGTACTTC |
| 737 | 157-TL704-TIL-CD8+_CD103+ | TIL | CD8+_CD103+ | TGTGCCAGCAGTTACTCCCTGTCGTCGGCGGTGGCGTACAA |
| | | | | TGAGCAGTTCTTC |
| 738 | 157-TL704-TIL-CD8+_CD103+ | TIL | CD8+_CD103+ | TGCAGTGCTAGAGATCTCGGACAGTACACAACAAATCAGCCCCAGCATTTT |
| 739 | 157-TL704-TIL-CD8+_CD103+ | TIL | CD8+_CD103+ | TGTGCCAGCAGCCCGGGCTACGGGGGCGCGACTGAAGCTTTCTTT |
| 740 | 157-TL704-TIL-CD8+_CD103+ | TIL | CD8+_CD103+ | TGTGCCAGCAGTCAAAGCGGGGGGCTATCCTACAATGAGCAGTTCTTC |
| 741 | 157-TL704-TIL-CD8+_CD103+ | TIL | CD8+_CD103+ | TGTGCCAGCAGCTTAGAGGGCGGATTGAACACTGAAGCTTTCTTT |
| 742 | 157-TL704-TIL-CD8+_CD103+ | TIL | CD8+_CD103+ | TGTGCCAGCAGCCCCCAGTATGAGCAGTTCTTC |
| 743 | 157-TL704-TIL-CD8+_CD103+ | TIL | CD8+_CD103+ | TGTGCCAGCTCACCAACAGGGGGCGAAGATACGCAGTATTTT |
| 744 | 158-TL704-TIL-CD8+_CD103− | TIL | CD8+_CD103− | TGCGCCAGCAGCCAAGATCGGGTTAGCAGGAACACCGGGGAGCTGTTTTTT |
| 745 | 158-TL704-TIL-CD8+_CD103− | TIL | CD8+_CD103− | TGTGCCAGCAGCCAAGCGAGCGGGCCCCAAGAGACCCAGTACTTC |
| 746 | 158-TL704-TIL-CD8+_CD103− | TIL | CD8+_CD103− | TGTGCCAGCAGTTTTTCGGCCGGGCTCAATCAGCCCCAGCATTTT |
| 747 | 158-TL704-TIL-CD8+_CD103− | TIL | CD8+_CD103− | TGTGCCAGCATAGGCGCTAGCGGTTACAATGAGCAGTTCTTC |
| 748 | 158-TL704-TIL-CD8+_CD103− | TIL | CD8+_CD103− | TGCGCCAGCAGCTCGGGACACGTCTATGGCTACACCTTC |
| 749 | 158-TL704-TIL-CD8+_CD103− | TIL | CD8+_CD103− | TGTGCCAGCAGTTTATTACTGCAATCCTACAATGAGCAGTTCTTC |
| 750 | 158-TL704-TIL-CD8+_CD103− | TIL | CD8+_CD103− | TGTGCCAGCAGCCGACTAGCGGGACCCTACGAGCAGTACTTC |
| 751 | 158-TL704-TIL-CD8+_CD103− | TIL | CD8+_CD103− | TGTGCCAGCAGTTACTTACAGGGGATGAACACTGAAGCTTTCTTT |
| 752 | 158-TL704-TIL-CD8+_CD103− | TIL | CD8+_CD103− | TGTGCCAGTAATGCATATGATAATTCACCCCTCCACTTT |
| 753 | 158-TL704-TIL-CD8+_CD103− | TIL | CD8+_CD103− | TGTGCCAGCAGTACGACCCACCCTAATCAGCCCCAGCATTTT |
| 754 | 158-TL704-TIL-CD8+_CD103− | TIL | CD8+_CD103− | TGTGCCAGCAGTCGGGACTTTCGCTACGAGCAGTACTTC |
| 755 | 158-TL704-TIL-CD8+_CD103− | TIL | CD8+_CD103− | TGTGCCAGCAGTTTGGTAGGGTATGGCTACAATGAGCAGTTCTTC |
| 756 | 158-TL704-TIL-CD8+_CD103− | TIL | CD8+_CD103− | TGTGCCAGCGGACTGGACTACGAGCAGTACTTC |
| 757 | 158-TL704-TIL-CD8+_CD103− | TIL | CD8+_CD103− | TGTGCCACCAGTGACTCGGGACGGTTCTCTGGGGCCAACGTCCTGACTTTC |
| 758 | 158-TL704-TIL-CD8+_CD103− | TIL | CD8+_CD103− | TGTGCCAGCAGCTTAGGGGCTACCACCGGGGAGCTGTTTTTT |
| 759 | 158-TL704-TIL-CD8+_CD103− | TIL | CD8+_CD103− | TGTGCCACCAGTGAGCAAGGGACTTTCATTCCCCAGCATTTT |
| 760 | 158-TL704-TIL-CD8+_CD103− | TIL | CD8+_CD103− | TGTGCCAGCTCACCAACAGGGGGCGAAGATACGCAGTATTTT |
| 761 | 158-TL704-TIL-CD8+_CD103− | TIL | CD8+_CD103− | TGTGCCAGCAGTGAAAGCTACGAGCAGTACTTC |
| 762 | 158-TL704-TIL-CD8+_CD103− | TIL | CD8+_CD103− | TGTGCCAGCAGCTCGTTCGGGGTACCTTCAGGGACCCAGTACTTC |
| 763 | 158-TL704-TIL-CD8+_CD103− | TIL | CD8+_CD103− | TGTGCCAGCAGCCCGACGGGGGGGCCCTACAATGAGCAGTTCTTC |
| 764 | 158-TL704-TIL-CD8+_CD103− | TIL | CD8+_CD103− | TGTGCCAGCAGTTACACGACTAGCGGCACCGGGGAGCTGTTTTTT |
| 765 | 158-TL704-TIL-CD8+_CD103− | TIL | CD8+_CD103− | TGTGCCACCAGCATGGTAGCGGGAGGTACCTACGAGCAGTACTTC |
| 766 | 158-TL704-TIL-CD8+_CD103− | TIL | CD8+_CD103− | TGTGCCAGCAGCTTGGGACTGGGCCTTCTTACGCAGTATTTT |
| 767 | 158-TL704-TIL-CD8+_CD103− | TIL | CD8+_CD103− | TGTGCCAGCAGCTCCCTGGACAGGGGCGACTACGAGCAGTACTTC |
| 768 | 158-TL704-TIL-CD8+_CD103− | TIL | CD8+_CD103− | TGTGCAAGTCGCTTACTTGGCAGCCCGGGTAACACTGAAGCTTTCTTT |
| 769 | 158-TL704-TIL-CD8+_CD103− | TIL | CD8+_CD103− | TGTGCCACCAGCCCCCGGGGGGCGGACAGGACCTATAA |
| | | | | CTATGGCTACACCTTC |
| 770 | 158-TL704-TIL-CD8+_CD103− | TIL | CD8+_CD103− | TGTGCCAGCACGAGGTGGGACAGGGAGCCCGAAGGGTACGAGCAGTACTTC |
| 771 | 158-TL704-TIL-CD8+_CD103− | TIL | CD8+_CD103− | TGTGCCAGCAGTTTTTGGGGGATTACCTCCTACGAGCAGTACTTC |
| 772 | 158-TL704-TIL-CD8+_CD103− | TIL | CD8+_CD103− | TGTGCCAGCACCGAAGTTAGCGGGGGGACCCAAGAGACCCAGTACTTC |
| 773 | 158-TL704-TIL-CD8+_CD103− | TIL | CD8+_CD103− | TGTGCCAGCAGCTTCGGGACTAGCGGGTTCACAGATACGCAGTATTTT |
| 774 | 158-TL704-TIL-CD8+_CD103− | TIL | CD8+_CD103− | TGTGCCAGCAGCGGGGCGCGAGTGGAAAAAGAAAAACTGTTTTTT |
| 775 | 158-TL704-TIL-CD8+_CD103− | TIL | CD8+_CD103− | TGTGCCAGCAGCCCCCCCACTAGCGGCCAAGAGACCCAGTACTTC |
| 776 | 158-TL704-TIL-CD8+_CD103− | TIL | CD8+_CD103− | TGTGCCAGCAGTCCGTACGGGGACGCTTTGGGAGAGACCCAGTACTTC |
| 777 | 158-TL704-TIL-CD8+_CD103− | TIL | CD8+_CD103− | TGTGCCAGCAGCCAAGATTGGGGGACTAGCGGGGGGTCTTGGAGTGA |
| | | | | GCAGTTCTTC |
| 778 | 158-TL704-TIL-CD8+_CD103− | TIL | CD8+_CD103− | TGTGCCAGCGGGAAACGGACTAGTTGGCCTCGAGAGACCCAGTACTTC |
| 779 | 158-TL704-TIL-CD8+_CD103− | TIL | CD8+_CD103− | TGTGCCAGCAGCTGGACTAGCGGGGGGGCCAATGAGCAGTTCTTC |
| 780 | 158-TL704-TIL-CD8+_CD103− | TIL | CD8+_CD103− | TGTGCCAGCAGTTTCCGGGGACAGGTTCTGAACACCGGGGAGCTGTTTTTT |
| 781 | 158-TL704-TIL-CD8+_CD103− | TIL | CD8+_CD103− | TGTGCCAGCAGCTACGGCGGGGGCCATGAGCAGTTCTTC |
| 782 | 158-TL704-TIL-CD8+_CD103− | TIL | CD8+_CD103− | TGTGCCAGCAGCTTCCCCGGGAGGGAATACTATGGCTACACCTTC |
| 783 | 158-TL704-TIL-CD8+_CD103− | TIL | CD8+_CD103− | TGTGCCAGCAGTACCGACAGGGGACTAGCGGGGGTAGCG |
| | | | | GACGAGCAGTACTTC |
| 784 | 158-TL704-TIL-CD8+_CD103− | TIL | CD8+_CD103− | TGTGCCAGCAGCTTAGCGCCGCAAGATACGCAGTATTTT |
| 785 | 158-TL704-TIL-CD8+_CD103− | TIL | CD8+_CD103− | TGTGCCAGCAGCTTACATGGGACCAACACAGATACGCAGTATTTT |
| 786 | 158-TL704-TIL-CD8+_CD103− | TIL | CD8+_CD103− | TGTGCCAGCATAGACGGGAGCGAGACCCAGTACTTC |
| 787 | 158-TL704-TIL-CD8+_CD103− | TIL | CD8+_CD103− | TGTGCCAGTAGTATGGGGACGTGGGAAGACAATGAGCAGTTCTTC |
| 788 | 158-TL704-TIL-CD8+_CD103− | TIL | CD8+_CD103− | TGCAGTGCTAGAGATCGTGCGGGACAGGGGACAGAGACCCAGTACTTC |
| 789 | 158-TL704-TIL-CD8+_CD103− | TIL | CD8+_CD103− | TGCAGTGCCCGTCCGTTGGGACAAATCTACGAGCAGTACTTC |
| 790 | 158-TL704-TIL-CD8+_CD103− | TIL | CD8+_CD103− | TGTGCCAGCAGCTGGGACAGCTCCTACGAGCAGTACTTC |
| 791 | 158-TL704-TIL-CD8+_CD103− | TIL | CD8+_CD103− | TGTGCCAGCAGCTCCGGGACAGCTTGGGAGACCCAGTACTTC |
| 792 | 158-TL704-TIL-CD8+_CD103− | TIL | CD8+_CD103− | TGCAGCGTTTATGGGCAGAGTACCTACGAGCAGTACTTC |
| 793 | 158-TL704-TIL-CD8+_CD103− | TIL | CD8+_CD103− | TGCGCCAGCAGCCAGGAACCTCCGGACGATGGTCTTTACGAGCAGTACTTC |
| 794 | 158-TL704-TIL-CD8+_CD103− | TIL | CD8+_CD103− | TGTGCCAGCAGTCCCTTAGCGGGGGCCTACTACAATGAGCAGTTCTTC |
| 795 | 158-TL704-TIL-CD8+_CD103− | TIL | CD8+_CD103− | TGTGCCAGCAGCTTAGAGGGCGGATTGAACACTGAAGCTTTCTTT |
| 796 | 158-TL704-TIL-CD8+_CD103− | TIL | CD8+_CD103− | TGCGCCAGCAGCCACGAAGGGACCGATTCCACCTACTATAATTCACC |
| | | | | CCTCCACTTT |
| 797 | 158-TL704-TIL-CD8+_CD103− | TIL | CD8+_CD103− | TGCAGCGTTGAAATCCGGGGGCGAGTTTACGAGCAGTACTTC |
| 798 | 158-TL704-TIL-CD8+_CD103− | TIL | CD8+_CD103− | TGTGCCAGCAGCCTGGGGACAGGGGACAATGAGCAGTTCTTC |
| 799 | 158-TL704-TIL-CD8+_CD103− | TIL | CD8+_CD103− | TGTGCCAGCAGTTTAGCCAGGGCTAGCAATCAGCCCCAGCATTTT |
| 800 | 158-TL704-TIL-CD8+_CD103− | TIL | CD8+_CD103− | TGTGCCAGCAGCAAAGGGACGGGCAGGGACAACATTCAGTACTTC |
| 801 | 158-TL704-TIL-CD8+_CD103− | TIL | CD8+_CD103− | TGTGCCAGCAGCCACCAAGGTGCCCCGGCAAGTTCTTACGGCTACACCTTC |
| 802 | 158-TL704-TIL-CD8+_CD103− | TIL | CD8+_CD103− | TGTGCCAGCAGTTTTTCGGCGGGGCTCAATCAGCCCCAGCATTTT |
| 803 | 158-TL704-TIL-CD8+_CD103− | TIL | CD8+_CD103− | TGTGCCAGCTTCCCGGACGGGACCGGGGAGCTGTTTTTT |
| 804 | 158-TL704-TIL-CD8+_CD103− | TIL | CD8+_CD103− | TGCGCCAGCAGACAAGCGGGGGTTAATGAGCAGTTCTTC |
| 805 | 158-TL704-TIL-CD8+_CD103− | TIL | CD8+_CD103− | TGTGCCAGCTCACCACCGTTAGGGCATTTGAGCGTCGATGAGCAGTTCTTC |
| 806 | 158-TL704-TIL-CD8+_CD103− | TIL | CD8+_CD103− | TGTGCCAGCAGCGTCGCGGGGGGAGAGCAGTTCTTC |
| 807 | 158-TL704-TIL-CD8+_CD103− | TIL | CD8+_CD103− | TGTGCCAGCAGTACGTGGGGCGAAAAACTGTTTTTT |
| 808 | 158-TL704-TIL-CD8+_CD103− | TIL | CD8+_CD103− | TGTGCCAGCAAAGGGGGCGGCAATGAGCAGTTCTTC |
| 809 | 158-TL704-TIL-CD8+_CD103− | TIL | CD8+_CD103− | TGTGCCAGCAGCTTAGCCCGCCTAGCCCTGACCGGGGAGCTGTTTTTT |
| 810 | 158-TL704-TIL-CD8+_CD103− | TIL | CD8+_CD103− | TGTGCCAGCAGCTTAGGGGTAAGAACTGAAGCTTTCTTT |
| 811 | 158-TL704-TIL-CD8+_CD103− | TIL | CD8+_CD103− | TGTGCCAGCAGCCTCAATCGGGACCCCGACTACAATGAGCAGTTCTTC |
| 812 | 158-TL704-TIL-CD8+_CD103− | TIL | CD8+_CD103− | TGTGCCAGCACCCCCGGACTAGCGGGAGCGTACGAGCAGTACTTC |
| 813 | 158-TL704-TIL-CD8+_CD103− | TIL | CD8+_CD103− | TGTGCCAGCAGCTCCGCGGTAACGACCGCGCAGGGGGAGACCCAGTACTTC |
| 814 | 158-TL704-TIL-CD8+_CD103− | TIL | CD8+_CD103− | TGTGCCAGCAGTTTCCCGCGCGAAAGAGCGGTGAACACCGGGGAGCTGTTTTTT |
| 815 | 158-TL704-TIL-CD8+_CD103− | TIL | CD8+_CD103− | TGTGCCAGCAGTTTGGTAGTGGCGGGAGTGAGGAATGAGCAGTTCTTC |
| 816 | 158-TL704-TIL-CD8+_CD103− | TIL | CD8+_CD103− | TGTGCCATCAGTGAGTCGATATCACCGCTCAATGGCTACACCTTC |
| 817 | 158-TL704-TIL-CD8+_CD103− | TIL | CD8+_CD103− | TGTGCCAGCAGTTTATCCGGGACTGAAGCTTTCTTT |
| 818 | 158-TL704-TIL-CD8+_CD103− | TIL | CD8+_CD103− | TGTGCCAGCAGAGGACAGCTACAAGAGACCCAGTACTTC |
| 819 | 158-TL704-TIL-CD8+_CD103− | TIL | CD8+_CD103− | TGTGCCAGCAGCACCCTGAGTGCGGGAGTGATGCCAGATACGCAGTATTTT |
| 820 | 158-TL704-TIL-CD8+_CD103− | TIL | CD8+_CD103− | TGTGCCAGCAGCGGGACTAGCGGGAGGACCGGGGAGCTGTTTTTT |
| 821 | 158-TL704-TIL-CD8+_CD103− | TIL | CD8+_CD103− | TGTGCCAGCAGCCCACCCCGGGACAGGGGTGGGTACACCTCCTACAA |
| | | | | TGAGCAGTTCTTC |
| 822 | 158-TL704-TIL-CD8+_CD103− | TIL | CD8+_CD103− | TGTGCCAGCAGCGACCGACTAGCTGGGGAGCAGTACTTC |
| 823 | 158-TL704-TIL-CD8+_CD103− | TIL | CD8+_CD103− | TGTGCCAGCAGCGTAGTAGCGACTAGCGATGAGCAGTTCTTC |
| 824 | 158-TL704-TIL-CD8+_CD103− | TIL | CD8+_CD103− | TGTGCCAGCAGTAGCCTGAGGGTCTCCTACGAGCAGTACTTC |
| 825 | 158-TL704-TIL-CD8+_CD103− | TIL | CD8+_CD103− | TGTGCCAGCAGCCCAGCCGGACAGGGCTTTTCATCAGATACGCAGTATTTT |
| 826 | 158-TL704-TIL-CD8+_CD103− | TIL | CD8+_CD103− | TGTGCCAGCAGCGCAGGGAGAGGCACTGAAGCTTTCTTT |
| 827 | 158-TL704-TIL-CD8+_CD103− | TIL | CD8+_CD103− | TGTGCCAGCAGTCTATGGACTAGCGGAGCGATTCAGGATACGCAGTATTTT |
| 828 | 158-TL704-TIL-CD8+_CD103− | TIL | CD8+_CD103− | TGTGCCAGCAGCCATTTTGGGGGAGGGGTTTGGTCCTACGAGCAGTACTTC |
| 829 | 158-TL704-TIL-CD8+_CD103− | TIL | CD8+_CD103− | TGTGCCAGCAGCCTAGGACAGGGTATTGACACCGGGGAGCTGTTTTTT |
| 830 | 158-TL704-TIL-CD8+_CD103− | TIL | CD8+_CD103− | TGCAGCGTTACCGGGACTGATACCTACGAGCAGTACTTC |
| 831 | 158-TL704-TIL-CD8+_CD103− | TIL | CD8+_CD103− | TGTGCCAGCAGTTTTTCGCGACACTTCTCCTACGAGCAGTACTTC |
| 832 | 158-TL704-TIL-CD8+_CD103− | TIL | CD8+_CD103− | TGCAGTGCTAGACCTGCTACTAGCGGGTTGGGGGATGAGCAGTTCTTC |
| 833 | 158-TL704-TIL-CD8+_CD103− | TIL | CD8+_CD103− | TGTGCCAGCAGCGGGGACAGGGTGGAGACCCAGTACTTC |
| 834 | 158-TL704-TIL-CD8+_CD103− | TIL | CD8+_CD103− | TGTGCCAGCAGCTTAGCTAGCGGTCCCACAGATACGCAGTATTTT |
| 835 | 158-TL704-TIL-CD8+_CD103− | TIL | CD8+_CD103− | TGCGCCAGCAGCCAAGATAGGGGGGGGGGATACGAGCAGTACTTC |
| 836 | 158-TL704-TIL-CD8+_CD103− | TIL | CD8+_CD103− | TGTGCCAGCAGCTTAGGGGGCAGCTCCTCAGGGGATGGCTACACCTTC |
| 837 | 158-TL704-TIL-CD8+_CD103− | TIL | CD8+_CD103− | TGTGCCAGCAGCGTAGGAAGTACAGCAGACTACGAGCAGTACTTC |
| 838 | 158-TL704-TIL-CD8+_CD103− | TIL | CD8+_CD103− | TGTGCCAGCAGCTCCGGGACAGGGGGTAAAAATGAAAAACTGTTTTTT |
| 839 | 158-TL704-TIL-CD8+_CD103− | TIL | CD8+_CD103− | TGTGCCAGAGGCGGGTCAAGCACAGATACGCAGTATTTT |
| 840 | 158-TL704-TIL-CD8+_CD103− | TIL | CD8+_CD103− | TGTGCCAGCAGCAGCGAGAGGATAGCGGGAGGGCGACAAGAGACCCAG |
| | | | | TACTTC |
| 841 | 158-TL704-TIL-CD8+_CD103− | TIL | CD8+_CD103− | TGTGCCAGCAGCTCCCCTAGCGCCGAGCAGTACTTC |
| 842 | 158-TL704-TIL-CD8+_CD103− | TIL | CD8+_CD103− | TGTGCCAGCAGCTTAGCGGGAGGGCTTGAAGATGAGCAGTTCTTC |
| 843 | 158-TL704-TIL-CD8+_CD103− | TIL | CD8+_CD103− | TGCGCCAGCAGCCAAGATTTACAGGGGATGAACACTGAAGCTTTCTTT |
| 844 | 158-TL704-TIL-CD8+_CD103− | TIL | CD8+_CD103− | TGTGCCAGCAGCAACCGGACAGATAGCTCCTACAATGAGCAGTTCTTC |
| 845 | 158-TL704-TIL-CD8+_CD103− | TIL | CD8+_CD103− | TGTGCCAGCAGCCCAGGGGCTAGGTCGGGTGAGCAGTACTTC |
| 846 | 158-TL704-TIL-CD8+_CD103− | TIL | CD8+_CD103− | TGTGCCAGCAGTGATCTACCGGGACAGGGATACGAGCAGTACTTC |
| 847 | 158-TL704-TIL-CD8+_CD103− | TIL | CD8+_CD103− | TGTGCCAGTACCCCGACAGGGTTAAACACTGAAGCTTTCTTT |
| 848 | 158-TL704-TIL-CD8+_CD103− | TIL | CD8+_CD103− | TGTGCCAGCATGACGGGAGCCGGTAACTATGGCTACACCTTC |
| 849 | 158-TL704-TIL-CD8+_CD103− | TIL | CD8+_CD103− | TGTGCCAGCAGTTACTCGAGCGAACCTTTACAGGGAATGGGG |
| | | | | GCCTATGGCTACACCTTC |
| 850 | 158-TL704-TIL-CD8+_CD103− | TIL | CD8+_CD103− | TGCGCCAGCAGCCAAGCCAAGGGTGAACTTCATACGAACACC |
| | | | | GGGGAGCTGTTTTTT |
| 851 | 158-TL704-TIL-CD8+_CD103− | TIL | CD8+_CD103− | TGTGCCAGCAGCTTCCTGGGACTAGCGGACACAGATACGCAGTATTTT |
| 852 | 158-TL704-TIL-CD8+_CD103− | TIL | CD8+_CD103− | TGTGCCAGCAGCCTAACAGGGGGGGGGAATCAGCCCCAGCATTTT |
| 853 | 158-TL704-TIL-CD8+_CD103− | TIL | CD8+_CD103− | TGTGCCAGCAGCCAACTTAAGGGACAGGGGGTTGACTATGGCTACACCTTC |
| 854 | 158-TL704-TIL-CD8+_CD103− | TIL | CD8+_CD103− | TGTGCCAGCAGCTCCCAGACAGGGCTCACAGATACGCAGTATTTT |
| 855 | 158-TL704-TIL-CD8+_CD103− | TIL | CD8+_CD103− | TGCAGCGTTGAAGAAGGCGGGCCGCCGCCAAGCTCCTACAATGAG |
| | | | | CAGTTCTTC |
| 856 | 158-TL704-TIL-CD8+_CD103− | TIL | CD8+_CD103− | TGTGCCAGCAGCGAGGTCGGGGGAGGGGGACGAGCGAACACCG |
| | | | | GGGAGCTGTTTTTT |
| 857 | 158-TL704-TIL-CD8+_CD103− | TIL | CD8+_CD103− | TGTGCCACCAGTGATGGAGGGGACGCACATACCCAAGAGACCCAGTACTTC |
| 858 | 158-TL704-TIL-CD8+_CD103− | TIL | CD8+_CD103− | TGTGCCAGCAGAATTAGCGGGACCCACACAGATACGCAGTATTTT |
| 859 | 158-TL704-TIL-CD8+_CD103− | TIL | CD8+_CD103− | TGTGCCAGCAGCCCCGCCAAAGCCCATGGCTACACCTTC |
| 860 | 158-TL704-TIL-CD8+_CD103− | TIL | CD8+_CD103− | TGTGCCAGCAGTCCACAGACGGGACTCGACGAGACCCAGTACTTC |
| 861 | 158-TL704-TIL-CD8+_CD103− | TIL | CD8+_CD103− | TGCGCCAGCAGCTTCGACAGTAACACTGAAGCTTTCTTT |
| 862 | 158-TL704-TIL-CD8+_CD103− | TIL | CD8+_CD103− | TGTGCCAGCAGTTTGCAGGGTTACACCGGGGAGCTGTTTTTT |
| 863 | 158-TL704-TIL-CD8+_CD103− | TIL | CD8+_CD103− | TGTGCCAGCAGTTTACAGGAGACCTCCTACGAGCAGTACTTC |
| 864 | 158-TL704-TIL-CD8+_CD103− | TIL | CD8+_CD103− | TGTGCCAGCAGCGGTAATGAGCAGTTCTTC |
| 865 | 158-TL704-TIL-CD8+_CD103− | TIL | CD8+_CD103− | TGTGCCAGCAGCGTAGAGGGGGGGGAAGAGACCCAGTACTTC |
| 866 | 158-TL704-TIL-CD8+_CD103− | TIL | CD8+_CD103− | TGCAGCGTTGAGGTTGTAGGAGGGCCCGGGGAGCTGTTTTTT |
| 867 | 158-TL704-TIL-CD8+_CD103− | TIL | CD8+_CD103− | TGCGCCAGCAGCCAAGATTCGGGCAGGGGTTACGAGCAGTACTTC |
| 868 | 158-TL704-TIL-CD8+_CD103− | TIL | CD8+_CD103− | TGTGCCAGCAGCTTCCATTTTAAGGACGAGGAGCACACTGAAGCTTTCTTT |
| 869 | 158-TL704-TIL-CD8+_CD103− | TIL | CD8+_CD103− | TGTGCCAGCAGCCAAGAATGGATAGATACGCAGTATTTT |
| 870 | 158-TL704-TIL-CD8+_CD103− | TIL | CD8+_CD103− | TGTGCCAGCAGCTTAGATGATAGGGCCGGGGAGCTGTTTTTT |
| 871 | 158-TL704-TIL-CD8+_CD103− | TIL | CD8+_CD103− | TGTGCCAGCAGCTTAACCGGACAGTCCTACAATGAGCAGTTCTTC |
| 872 | 158-TL704-TIL-CD8+_CD103− | TIL | CD8+_CD103− | TGTGCCAGCAGCGTTAGACTAGCCTACAATGAGCAGTTCTTC |
| 873 | 158-TL704-TIL-CD8+_CD103− | TIL | CD8+_CD103− | TGTGCCAGCAGCTTGACAGGGATACAGCCCCAGCATTTT |
| 874 | 158-TL704-TIL-CD8+_CD103− | TIL | CD8+_CD103− | TGCAGCGTTGGGACCCTCGGGACAAGTAGTACATACTATGGCTACACCTTC |
| 875 | 158-TL704-TIL-CD8+_CD103− | TIL | CD8+_CD103− | TGTGCCAGCAGCGCTTTTTTTGGAGTGTTAGCAGATACGCAGTATTTT |
| 876 | 158-TL704-TIL-CD8+_CD103− | TIL | CD8+_CD103− | TGTGCCACCAGTAACGGGGACACTGAAGCTTTCTTT |
| 877 | 158-TL704-TIL-CD8+_CD103− | TIL | CD8+_CD103− | TGCGCCAGCAGCCAAGACAGGGATCTCAGCACCGGGGAGCTGTTTTTT |
| 878 | 158-TL704-TIL-CD8+_CD103− | TIL | CD8+_CD103− | TGTGCCAGCAATCCACAGGGGAGTGAGCAGTTCTTC |
| 879 | 158-TL704-TIL-CD8+_CD103− | TIL | CD8+_CD103− | TGTGCCAGCAGCTTAACAGGGACAGCTACCTACGAGCAGTACTTC |
| 880 | 158-TL704-TIL-CD8+_CD103− | TIL | CD8+_CD103− | TGCAGTGTCCGGGACAGGGCTTCCGGGGGGGAGACCCAGTACTTC |
| 881 | 158-TL704-TIL-CD8+_CD103− | TIL | CD8+_CD103− | TGTGCCAGCAGTTCGAACGACGGGATCTCTGGAAACACCATATATTTT |
| 882 | 158-TL704-TIL-CD8+_CD103− | TIL | CD8+_CD103− | TGTGCCAGCAGCTTAGGGACTAGCGGGATACAAGAGACCCAGTACTTC |
| 883 | 158-TL704-TIL-CD8+_CD103− | TIL | CD8+_CD103− | TGTGCCAGCAGCTCAACCGGGACAGAAGAGACCCAGTACTTC |
| 884 | 158-TL704-TIL-CD8+_CD103− | TIL | CD8+_CD103− | TGTGCCAGCAGCCCCGGGACTAGACCGATAGAGATGTTGAAC |
| | | | | GAGCAGTACTTC |
| 885 | 158-TL704-TIL-CD8+_CD103− | TIL | CD8+_CD103− | TGTGCCTGGAGTCCGGGGACTACCTGGTTGCAGTACTTC |
| 886 | 158-TL704-TIL-CD8+_CD103− | TIL | CD8+_CD103− | TGTGCCAGCAGCGTAGGACTAGCGGGGGTTGAAAATGAGCAGTTCTTC |
| 887 | 158-TL704-TIL-CD8+_CD103− | TIL | CD8+_CD103− | TGTGCCAGCAGCTCGCCACTAGGTGATGAGCAGTTCTTC |
| 888 | 158-TL704-TIL-CD8+_CD103− | TIL | CD8+_CD103− | TGTGCCAGCAGCTTAGAATTCTCCTTCCAAGAGACCCAGTACTTC |
| 889 | 158-TL704-TIL-CD8+_CD103− | TIL | CD8+_CD103− | TGTGCCAGCAGTTTGGGGACTAGCGGGATCTACAATGAGCAGTTCTTC |
| 890 | 158-TL704-TIL-CD8+_CD103− | TIL | CD8+_CD103− | TGTGCCAGCAGCCAATATTCATTAGGGCAGGGAGGCGACGAGCAGTACTTC |
| 891 | 158-TL704-TIL-CD8+_CD103− | TIL | CD8+_CD103− | TGCAGTGCTAGAGGCCCCGGACAGGGCAATGAGCAGTTCTTC |
| 892 | 158-TL704-TIL-CD8+_CD103− | TIL | CD8+_CD103− | TGTGCCAGCAGCTTGGGGGGCCAGTCCTACGAGCAGTACTTC |
| 893 | 158-TL704-TIL-CD8+_CD103− | TIL | CD8+_CD103− | TGTGCCAGCAGACCAGCGTACTATGGCTACACCTTC |
| 894 | 158-TL704-TIL-CD8+_CD103− | TIL | CD8+_CD103− | TGTGCCAGCAACGCGCGCGGCCTTTCGGGACAGGGGCCCACCGGGG |
| | | | | AGCTGTTTTTT |
| 895 | 158-TL704-TIL-CD8+_CD103− | TIL | CD8+_CD103− | TGTGCCAGCAGCGCGGGACCCACGGGACTAGCGATCCACTACGAGC |
| | | | | AGTACTTC |
| 896 | 158-TL704-TIL-CD8+_CD103− | TIL | CD8+_CD103− | TGTGCCAGCAGTTCATCACTAGCGGGGGGCCGAGGGGAGCAGTTCTTC |
| 897 | 158-TL704-TIL-CD8+_CD103− | TIL | CD8+_CD103− | TGTGCCATCAGTGAGTCGGCCCCCGGGTGGACAGGGAACACTGAAGCT |
| | | | | TTCTTT |
| 898 | 158-TL704-TIL-CD8+_CD103− | TIL | CD8+_CD103− | TGCGCCAGCAGTCGGGACAGCTCGAACACTGAAGCTTTCTTT |
| 899 | 158-TL704-TIL-CD8+_CD103− | TIL | CD8+_CD103− | TGTGCCAGCAGTCCCACAGGGTACGAGGAAGAGACCCAGTACTTC |
| 900 | 158-TL704-TIL-CD8+_CD103− | TIL | CD8+_CD103− | TGTGCCAGCAGTTACTCCGGACCTGATATAGCCAAAAACATTCAGTACTTC |
| 901 | 158-TL704-TIL-CD8+_CD103− | TIL | CD8+_CD103− | TGCAGCGTTTTAGAGCGGACTAGCGTGGTCACAGATACGCAGTATTTT |
| 902 | 158-TL704-TIL-CD8+_CD103− | TIL | CD8+_CD103− | TGCGCCAGCAGCTTTTCGGACGGGAGGACGGATACGCAGTATTTT |
| 903 | 158-TL704-TIL-CD8+_CD103− | TIL | CD8+_CD103− | TGCAGTGCTAGAGTCACCCGGGACAGGGCTCAAGAGACCCAGTACTTC |
| 904 | 158-TL704-TIL-CD8+_CD103− | TIL | CD8+_CD103− | TGTGCCAGCAGTGCGACTAGCGGGGGCACCGGAGAGACCCAGTACTTC |
| 905 | 158-TL704-TIL-CD8+_CD103− | TIL | CD8+_CD103− | TGCGCCAGCAGCCAAGACCGTTACCGATCACCCCTCCACTTT |
| 906 | 158-TL704-TIL-CD8+_CD103− | TIL | CD8+_CD103− | TGTGCCAGCAGCTTCGTGGAGGCCACTGAAGCTTTCTTT |
| 907 | 158-TL704-TIL-CD8+_CD103− | TIL | CD8+_CD103− | TGCAGCGTTGAAGATACGGCGCACGAGCAGTACTTC |
| 908 | 158-TL704-TIL-CD8+_CD103− | TIL | CD8+_CD103− | TGTGCCAGCAGCCCTAGCGGGGAACCCTACGAGCAGTACTTC |
| 909 | 158-TL704-TIL-CD8+_CD103− | TIL | CD8+_CD103− | TGTGCCAGCAGTGTAGCTGGCACAGATACGCAGTATTTT |
| 910 | 158-TL704-TIL-CD8+_CD103− | TIL | CD8+_CD103− | TGTGCCAGCAGCTTAGACGCCCCCGGGCCAATGAACACTGAAGCTTTCTTT |
| 911 | 158-TL704-TIL-CD8+_CD103− | TIL | CD8+_CD103− | TGCGCCAGCAGCCAATTCCGGGTTTGGAATTCACCCCTCCACTTT |
| 912 | 158-TL704-TIL-CD8+_CD103− | TIL | CD8+_CD103− | TGTGCCACCGGAGGGACTAGCGGGAGGGCCGGGGGGACCGGGGAG |
| | | | | CTGTTTTTT |
| 913 | 158-TL704-TIL-CD8+_CD103− | TIL | CD8+_CD103− | TGTGCCAGCTCACCATTGGCTATCGACGAGCAGTACTTC |
| 914 | 158-TL704-TIL-CD8+_CD103− | TIL | CD8+_CD103− | TGCAGTGCTAGAGATCGGCTAGCGGGAAAAAGGGGGGCA |
| | | | | GATACGCAGTATTTT |
| 915 | 158-TL704-TIL-CD8+_CD103− | TIL | CD8+_CD103− | TGCAGTGCTAGAGCCCGGGACAGCGCCTACGAGCAGTACTTC |
| 916 | 158-TL704-TIL-CD8+_CD103− | TIL | CD8+_CD103− | TGTGCCAGCAGTTCCCCGCTTGAGGAGCAGTACTTC |
| 917 | 158-TL704-TIL-CD8+_CD103− | TIL | CD8+_CD103− | TGCGCCAGCAGCCAAGATTTGTCTGGGGCCAACGTCCTGACTTTC |
| 918 | 158-TL704-TIL-CD8+_CD103− | TIL | CD8+_CD103− | TGTGCCAGCAGTTTGACAGGCCTCTCCTACGAGCAGTACTTC |
| 919 | 158-TL704-TIL-CD8+_CD103− | TIL | CD8+_CD103− | TGTGCCAGCATTCCACTAGCGTCAGCCTCCTACAATGAGCAGTTCTTC |
| 920 | 158-TL704-TIL-CD8+_CD103− | TIL | CD8+_CD103− | TGTGCCAGCAGCTTACTACCCGGTCAGCCTGACAATGAGCAGTTCTTC |
| 921 | 158-TL704-TIL-CD8+_CD103− | TIL | CD8+_CD103− | TGCAGTGCTGGAGGGGAAACGCAGTATTTT |
| 922 | 158-TL704-TIL-CD8+_CD103− | TIL | CD8+_CD103− | TGCAGCGCTGCCGATTCCGAGCAGTACTTC |
| 923 | 158-TL704-TIL-CD8+_CD103− | TIL | CD8+_CD103− | TGTGCCATCAGTGAGGGTCTGGGGTTCGAGCAGTACTTC |
| 924 | 158-TL704-TIL-CD8+_CD103− | TIL | CD8+_CD103− | TGCAGCGTAGTCGGACTAGCGGACACCTCCTACGAGCAGTACTTC |
| 925 | 158-TL704-TIL-CD8+_CD103− | TIL | CD8+_CD103− | TGTGCCATCACTCCCGGACCGGGACAGAGCTCCTACGAGCAGTACTTC |
| 926 | 158-TL704-TIL-CD8+_CD103− | TIL | CD8+_CD103− | TGCGCCAGCAGCCAAGATCGGGGGGCCCAAGAGACCCAGTACTTC |
| 927 | 158-TL704-TIL-CD8+_CD103− | TIL | CD8+_CD103− | TGCGCCAGCAGCTTGGCCCCGGGACAGCCAGACAATGAGCAGTTCTTC |
| 928 | 158-TL704-TIL-CD8+_CD103− | TIL | CD8+_CD103− | TGTGCCAGCAGCGCCGAATTGGACAGGGGTGACTATGGCTACACCTTC |
| 929 | 158-TL704-TIL-CD8+_CD103− | TIL | CD8+_CD103− | TGCGCCAGCAGCTTGGATGTCGGGAACATTCAGTACTTC |
| 930 | 158-TL704-TIL-CD8+_CD103− | TIL | CD8+_CD103− | TGCAGCGTAGGTGCAGGGAGGTATGTTGAGCAGTTCTTC |
| 931 | 158-TL704-TIL-CD8+_CD103− | TIL | CD8+_CD103− | TGTGCCAGCAGTTTACGGCTGTCGGGAGATACGCAGTATTTT |
| 932 | 158-TL704-TIL-CD8+_CD103− | TIL | CD8+_CD103− | TGTGCCAGCAGTGATCGACAGGAGATCTCTGGAAACACCATATATTTT |
| 933 | 158-TL704-TIL-CD8+_CD103− | TIL | CD8+_CD103− | TGTGCCAGCAGTTTTCGGGGGATCGAGGACAATGAGCAGTTCTTC |
| 934 | 158-TL704-TIL-CD8+_CD103− | TIL | CD8+_CD103− | TGTGCCAGCAGTTACTCAGGGATCAACGAGCAGTACTTC |
| 935 | 158-TL704-TIL-CD8+_CD103− | TIL | CD8+_CD103− | TGTGCCAGCAGCCCATTGTCTAGTAGCCACAATGAGCAGTTCTTC |
| 936 | 158-TL704-TIL-CD8+_CD103− | TIL | CD8+_CD103− | TGTGCCAGTAGTATCCGGCAAGCAACTAATGAAAAACTGTTTTTT |
| 937 | 158-TL704-TIL-CD8+_CD103− | TIL | CD8+_CD103− | TGCGCCAGCAGTGAGCTGGACACCAGCTCCTACGAGCAGTACTTC |
| 938 | 158-TL704-TIL-CD8+_CD103− | TIL | CD8+_CD103− | TGTGCCAGCAGCTTGGGGGACAGGGCCTACAATGAGCAGTTCTTC |
| 939 | 158-TL704-TIL-CD8+_CD103− | TIL | CD8+_CD103− | TGTGCCAGCAGCTGGGGACTAGCGGGAGGCCCGAACACCGGGGAG |
| | | | | CTGTTTTTT |
| 940 | 158-TL704-TIL-CD8+_CD103− | TIL | CD8+_CD103− | TGTGCCTGCAGGAGGACGGGAGGAGGCAATTCACCCCTCCACTTT |
| 941 | 158-TL704-TIL-CD8+_CD103− | TIL | CD8+_CD103− | TGTGCCAGCAGCTTAGAGGGGGATAATCAGCCCCAGCATTTT |
| 942 | 158-TL704-TIL-CD8+_CD103− | TIL | CD8+_CD103− | TGCGCCAGCAGCGACGGACTAGCGGGGCGTCCTCCAGAGACCCAGTACTTC |
| 943 | 158-TL704-TIL-CD8+_CD103− | TIL | CD8+_CD103− | TGTGCCAGCAGCTTAGGAGCGGGGGCCACAGATACGCAGTATTTT |
| 944 | 158-TL704-TIL-CD8+_CD103− | TIL | CD8+_CD103− | TGTGCCAGCAGTTCGGGACAGGGGCTTTCCAATGAGCAGTTCTTC |
| 945 | 158-TL704-TIL-CD8+_CD103− | TIL | CD8+_CD103− | TGTGCCAGCAGTTTGGGTTACGGAGGTGCAGGGCTGTTTTTT |
| 946 | 158-TL704-TIL-CD8+_CD103− | TIL | CD8+_CD103− | TGTGCCAGCAGCTTAGTAGGGGACGGGACTAGCGTTTACAAT |
| | | | | GAGCAGTTCTTC |
| 947 | 158-TL704-TIL-CD8+_CD103− | TIL | CD8+_CD103− | TGTGCCAGCAGTCAGACAGGAAACTACGAGCAGTACTTC |
| 948 | 158-TL704-TIL-CD8+_CD103− | TIL | CD8+_CD103− | TGCGCCAGCAGCCAAGAAGGGGGACTAACGTCAGATACGCAGTATTTT |
| 949 | 158-TL704-TIL-CD8+_CD103− | TIL | CD8+_CD103− | TGCAGCGTTGAACTACGGCTTGGGGAGCTGTTTTTT |
| 950 | 158-TL704-TIL-CD8+_CD103− | TIL | CD8+_CD103− | TGCGCCAGCAGCCAAGATAGGGCGGGAACGGAGACCCAGTACTTC |
| 951 | 158-TL704-TIL-CD8+_CD103− | TIL | CD8+_CD103− | TGTGCCAGCAGTCCTCAGGAGGACTCCTATAATTCACCCCTCCACTTT |
| 952 | 158-TL704-TIL-CD8+_CD103− | TIL | CD8+_CD103− | TGTGCCAGCAGTGGGGGGGACAACTCCTACTACGAGCAGTACTTC |
| 953 | 158-TL704-TIL-CD8+_CD103− | TIL | CD8+_CD103− | TGTGCCACCAGTGATTTAAGCGGGTGGAACACCGGGGAGCTGTTTTTT |
| 954 | 158-TL704-TIL-CD8+_CD103− | TIL | CD8+_CD103− | TGTGCCAGCAGCCGCTCAGGGATCTATAATTCACCCCTCCACTTT |
| 955 | 158-TL704-TIL-CD8+_CD103− | TIL | CD8+_CD103− | TGTGCCAGCAGTTTAGGGGGGATAGCGGGAGAGAATGAGCAGTTCTTC |
| 956 | 158-TL704-TIL-CD8+_CD103− | TIL | CD8+_CD103− | TGCAGTGCCACGAATGCAGGGGGCGGTCAATTGGGGGAGCAGTACTTC |
| 957 | 158-TL704-TIL-CD8+_CD103− | TIL | CD8+_CD103− | TGCAGCGTTGATGCCCGGGACAGGGTTGAAGAGACCCAGTACTTC |
| 958 | 158-TL704-TIL-CD8+_CD103− | TIL | CD8+_CD103− | TGTGCCACCAGCAGCTACGGCACAGATACGCAGTATTTT |
| 959 | 158-TL704-TIL-CD8+_CD103− | TIL | CD8+_CD103− | TGTGCCATCAGTGTCCGCGGGACACCTAGACGCTACGAGCAGTACTTC |
| 960 | 158-TL704-TIL-CD8+_CD103− | TIL | CD8+_CD103− | TGTGCCAGCAGCTCCGCGGGGAGCTCCTACAATGAGCAGTTCTTC |
| 961 | 158-TL704-TIL-CD8+_CD103− | TIL | CD8+_CD103− | TGTGCCAGCAGCTTGGTGGATCTCAACACTGAAGCTTTCTTT |
| 962 | 158-TL704-TIL-CD8+_CD103− | TIL | CD8+_CD103− | TGCGCCAGCAGCCAAGATCTACGGGGGGGAAACTACGAGCAGTACTTC |
| 963 | 158-TL704-TIL-CD8+_CD103− | TIL | CD8+_CD103− | TGCGCCAGCAGCTTACAGGGGGCTGGGAACACTGAAGCTTTCTTT |
| 964 | 158-TL704-TIL-CD8+_CD103− | TIL | CD8+_CD103− | TGCAGTGCTAGAGATCCAGGTGGTGTGGTCTACAATGAGCAGTTCTTC |
| 965 | 158-TL704-TIL-CD8+_CD103− | TIL | CD8+_CD103− | TGTGCCAGCAGCTTAAACACAGGGATATTTACCGGGGAGCTGTTTTTT |
| 966 | 158-TL704-TIL-CD8+_CD103− | TIL | CD8+_CD103− | TGTGCCAGCAGCTTAGAAATTGCCACTGAAGCTTTCTTT |
| 967 | 158-TL704-TIL-CD8+_CD103− | TIL | CD8+_CD103− | TGTGCCAGCAGTTACTCGATCGGGAGGAAGTTCTTC |
| 968 | 172-TL720-TIL-CD8+_CD103+ | TIL | CD8+_CD103+ | TGTGCCAGCAGCAACGACATGACACCTGGGTGGATCTTC |
| 969 | 172-TL720-TIL-CD8+_CD103+ | TIL | CD8+_CD103+ | TGTGCCAGCTCTGACTCCCGGGAGGGGGAGCGAGGAACTGAAGCTTTCTTT |
| 970 | 172-TL720-TIL-CD8+_CD103+ | TIL | CD8+_CD103+ | TGTGCCAGTAGTATAGACGGCACTTCCTACGAGCAGTACTTC |
| 971 | 172-TL720-TIL-CD8+_CD103+ | TIL | CD8+_CD103+ | TGTGCCAGCAGCCCCTTTACTAGCGGGAACACCGGGGAGCTGTTTTTT |
| 972 | 172-TL720-TIL-CD8+_CD103+ | TIL | CD8+_CD103+ | TGTGCCAGCAGTTCATCTTCTACCGGGGGGGCTAACGGGGAGCTGTTTTTT |
| 973 | 172-TL720-TIL-CD8+_CD103+ | TIL | CD8+_CD103+ | TGTGCCACCATAGGGGGGGGGACAGATACGCAGTATTTT |
| 974 | 172-TL720-TIL-CD8+_CD103+ | TIL | CD8+_CD103+ | TGTGCCAGCAGTGAAGAAAACTACTCTGGAAACACCATATATTTT |
| 975 | 172-TL720-TIL-CD8+_CD103+ | TIL | CD8+_CD103+ | TGTGCCAGCAGTTTAACGGGGCCGAACACTGAAGCTTTCTTT |
| 976 | 172-TL720-TIL-CD8+_CD103+ | TIL | CD8+_CD103+ | TGTGCCAGCAGCTTGACGGGGACCGGAGTGGAGCAGTACTTC |
| 977 | 172-TL720-TIL-CD8+_CD103+ | TIL | CD8+_CD103+ | TGTGCCAGCAGCCCAGGAGGGGGGGGAGCCTACGAGCAGTACTTC |
| 978 | 172-TL720-TIL-CD8+_CD103+ | TIL | CD8+_CD103+ | TGTGCCAGCAGCCAAGATCTGGCAGGGTTCGCCTACGAGCAGTACTTC |
| 979 | 172-TL720-TIL-CD8+_CD103+ | TIL | CD8+_CD103+ | TGTGCCAGCAGTGCCGGGACCTACGAGCAGTACTTC |
| 980 | 172-TL720-TIL-CD8+_CD103+ | TIL | CD8+_CD103+ | TGTGCCAGCAGCTTTGGGACAGCCTACAATGAGCAGTTCTTC |
| 981 | 172-TL720-TIL-CD8+_CD103+ | TIL | CD8+_CD103+ | TGTGCCAGCAGAACCGGGACTAGCGGGGGTCTTGAGCAGTTCTTC |
| 982 | 172-TL720-TIL-CD8+_CD103+ | TIL | CD8+_CD103+ | TGTGCCAGCAGTCCCGCCGGGGGCGGGGAGCTGTTTTTT |
| 983 | 172-TL720-TIL-CD8+_CD103+ | TIL | CD8+_CD103+ | TGTGCCAGCAGCTTGGAACAGGGGGTATACTATGGCTACACCTTC |
| 984 | 172-TL720-TIL-CD8+_CD103+ | TIL | CD8+_CD103+ | TGTGCCAGCAATAGAGACACCGCCTCAAATGAGCAGTTCTTC |
| 985 | 172-TL720-TIL-CD8+_CD103+ | TIL | CD8+_CD103+ | TGCAGTGCTCCATTTGGGACCAATGAGCAGTTCTTC |
| 986 | 172-TL720-TIL-CD8+_CD103+ | TIL | CD8+_CD103+ | TGCGCCAGCAGACCGGGCCCCAACACCGGGGAGCTGTTTTTT |
| 987 | 172-TL720-TIL-CD8+_CD103+ | TIL | CD8+_CD103+ | TGTGCCAGCAGCTTGGGGGAGAACTATGGCTACACCTTC |
| 988 | 172-TL720-TIL-CD8+_CD103+ | TIL | CD8+_CD103+ | TGCGCCAGCAGTGAGGACGGTATGAACACTGAAGCTTTCTTT |
| 989 | 172-TL720-TIL-CD8+_CD103+ | TIL | CD8+_CD103+ | TGTGCCAGCAGAACGGGTTATCCCAATGGCTACACCTTC |
| 990 | 172-TL720-TIL-CD8+_CD103+ | TIL | CD8+_CD103+ | TGCGCCAGCAGCCAAGACGGAGCCCGCTACAATGAGCAGTTCTTC |
| 991 | 172-TL720-TIL-CD8+_CD103+ | TIL | CD8+_CD103+ | TGTGCCAGCAGGACAGGGTTACAAGAGACCCAGTACTTC |
| 992 | 172-TL720-TIL-CD8+_CD103+ | TIL | CD8+_CD103+ | TGTGCCAGCAGCCAAGGATGGACTAGCGGGGCGACGCAGTATTTT |
| 993 | 172-TL720-TIL-CD8+_CD103+ | TIL | CD8+_CD103+ | TGTGCCAGCAGCCAGGGACAGGGGGACGAGCAGTACTTC |
| 994 | 172-TL720-TIL-CD8+_CD103+ | TIL | CD8+_CD103+ | TGTGCCAGCAGTTACTCGAGTCTGGGGACTAATGAAAAACTGTTTTTT |
| 995 | 172-TL720-TIL-CD8+_CD103+ | TIL | CD8+_CD103+ | TGTGCCAGCAGCTTAAACGGCGCTTGGCGGGCGAACTACAATGAGCAG |
| | | | | TTCTTC |
| 996 | 172-TL720-TIL-CD8+_CD103+ | TIL | CD8+_CD103+ | TGCGCCAGCAGCCCGATAGGGACAGGGGAAAACATTCAGTACTTC |
| 997 | 172-TL720-TIL-CD8+_CD103+ | TIL | CD8+_CD103+ | TGTGCCAGCAGCCTTTCGGGCGGGGGGGACAATGAGCAGTTCTTC |
| 998 | 172-TL720-TIL-CD8+_CD103+ | TIL | CD8+_CD103+ | TGTGCCAGCAGCTACACAGATACGCAGTATTTT |
| 999 | 172-TL720-TIL-CD8+_CD103+ | TIL | CD8+_CD103+ | TGTGCCACCAGCAGTGACAGGGGGCTTGACACTGAAGCTTTCTTT |
| 1000 | 172-TL720-TIL-CD8+_CD103+ | TIL | CD8+_CD103+ | TGTGCCAGCAGCCCATCCGGGGGGGCAGGAGCCTACGAGCAGTACTTC |
| 1001 | 172-TL720-TIL-CD8+_CD103+ | TIL | CD8+_CD103+ | TGTGCCAGCAGCTTCCCCCTCGGGCAGAACACTGAAGCTTTCTTT |
| 1002 | 172-TL720-TIL-CD8+_CD103+ | TIL | CD8+_CD103+ | TGTGCCAGCAGTGACAGCCAATTCACAGATACGCAGTATTTT |
| 1003 | 172-TL720-TIL-CD8+_CD103+ | TIL | CD8+_CD103+ | TGCAGTGCTTGGCGGACCGGGACAGGAGAGAGAAACACTGAAGCTTTCTTT |
| 1004 | 172-TL720-TIL-CD8+_CD103+ | TIL | CD8+_CD103+ | TGCGCCAGCAGCTCCGACAGCTCCTATAATTCACCCCTCCACTTT |
| 1005 | 172-TL720-TIL-CD8+_CD103+ | TIL | CD8+_CD103+ | TGCGCCAGCAGCCAAGAAAGCGGGAGTTACTACGAGCAGTACTTC |
| 1006 | 172-TL720-TIL-CD8+_CD103+ | TIL | CD8+_CD103+ | TGTGCCAGCAGCCCTGGCTCTAGCGGGGCCGACGAGCAGTACTTC |
| 1007 | 172-TL720-TIL-CD8+_CD103+ | TIL | CD8+_CD103+ | TGTGCCAGCAGCCGCACTAGCGGCCCGTACAATGAGCAGTTCTTC |
| 1008 | 172-TL720-TIL-CD8+_CD103+ | TIL | CD8+_CD103+ | TGTGCCAGCAGCCCCGGGGGAGGGGGCTGGTACAATGAGCAGTTCTTC |
| 1009 | 172-TL720-TIL-CD8+_CD103+ | TIL | CD8+_CD103+ | TGTGCCAGCAGCCCTGGGACTAGCGGAGCCTACAATGAGCAGTTCTTC |
| 1010 | 172-TL720-TIL-CD8+_CD103+ | TIL | CD8+_CD103+ | TGTGCCAGCAGTGTGCTGGACAATGAGCAGTTCTTC |
| 1011 | 172-TL720-TIL-CD8+_CD103+ | TIL | CD8+_CD103+ | TGTGCCAGCAGTTACGGGACAGCCAACAATGAGCAGTTCTTC |
| 1012 | 172-TL720-TIL-CD8+_CD103+ | TIL | CD8+_CD103+ | TGTGCCAGCAGTCCCCCCGATACCTACAATGAGCAGTTCTTC |
| 1013 | 172-TL720-TIL-CD8+_CD103+ | TIL | CD8+_CD103+ | TGTGCCAGCAGTGGGACTGGGGCGATGTGGTACTTC |
| 1014 | 172-TL720-TIL-CD8+_CD103+ | TIL | CD8+_CD103+ | TGTGCCAGCAGGAGACAGGGAGACTACGAGCAGTACTTC |
| 1015 | 172-TL720-TIL-CD8+_CD103+ | TIL | CD8+_CD103+ | TGTGCCAGCAGTCCCGAGGGTAGCGGACTCTACGAGCAGTACTTC |
| 1016 | 172-TL720-TIL-CD8+_CD103+ | TIL | CD8+_CD103+ | TGTGCCAGCAGCTGGGAAGGCTCCTACGAGCAGTACTTC |
| 1017 | 172-TL720-TIL-CD8+_CD103+ | TIL | CD8+_CD103+ | TGCAGTGCCGCCTCGCTTCGACTAGCGGGGGGTTGGAATGAGCAGTTCTTC |
| 1018 | 172-TL720-TIL-CD8+_CD103+ | TIL | CD8+_CD103+ | TGTGCCAGCAGCAAAGCCGAAAAGCAAAGGGACAGGGTTCCCTGGGAGCAG |
| | | | | TACTTC |
| 1019 | 172-TL720-TIL-CD8+_CD103+ | TIL | CD8+_CD103+ | TGTGCCAGCAGACCCTCGGGGGGGGTGGAGACCCAGTACTTC |
| 1020 | 172-TL720-TIL-CD8+_CD103+ | TIL | CD8+_CD103+ | TGCGCCAGCAGCCAAGATCGGGGGGGGGCAAGGGAGCAGTACTTC |
| 1021 | 172-TL720-TIL-CD8+_CD103+ | TIL | CD8+_CD103+ | TGTGCCAGCAGCCCGACTCCTAGCACAGATACGCAGTATTTT |
| 1022 | 172-TL720-TIL-CD8+_CD103+ | TIL | CD8+_CD103+ | TGTGCCAGCAGCTTTGGCGGGGGGGCCAATGAGCAGTTCTTC |
| 1023 | 172-TL720-TIL-CD8+_CD103+ | TIL | CD8+_CD103+ | TGTGCCAGCAGCTTAACGGGAGGGACAGGGGGCACTGAAGCTTTCTTT |
| 1024 | 172-TL720-TIL-CD8+_CD103+ | TIL | CD8+_CD103+ | TGCGCCAGCAGCCAAGATGTGGCAGGGGAGGGGCAGGAGCAGTTCTTC |
| 1025 | 172-TL720-TIL-CD8+_CD103+ | TIL | CD8+_CD103+ | TGTGCCAGCAGCCTTTATAACGGGGAGCTGTTTTTT |
| 1026 | 172-TL720-TIL-CD8+_CD103+ | TIL | CD8+_CD103+ | TGTGCCAGCAGCTCCTCAGGGTGGGAGCAGTACTTC |
| 1027 | 172-TL720-TIL-CD8+_CD103+ | TIL | CD8+_CD103+ | TGCGCCAGCAGCTTGGGGAATGAAGCTTTCTTT |
| 1028 | 172-TL720-TIL-CD8+_CD103+ | TIL | CD8+_CD103+ | TGTGCCAGCAGCCAAGATTCAGGGATGAACAATGAGCAGTTCTTC |
| 1029 | 172-TL720-TIL-CD8+_CD103+ | TIL | CD8+_CD103+ | TGTGCCAGCAGTTCATCTTCGGGGGACGGGGGTAAAGATGAGCAGTTCTTC |
| 1030 | 172-TL720-TIL-CD8+_CD103+ | TIL | CD8+_CD103+ | TGTGCCAGCAGTAATTTCCAGGGGCACTACGAGCAGTACTTC |
| 1031 | 172-TL720-TIL-CD8+_CD103+ | TIL | CD8+_CD103+ | TGTGCCAGCAGCGTCGGGGGGGACTACAATGAGCAGTTCTTC |
| 1032 | 172-TL720-TIL-CD8+_CD103+ | TIL | CD8+_CD103+ | TGTGCCAGCAGTTGGACAGGGAATGAGCAGTTCTTC |
| 1033 | 172-TL720-TIL-CD8+_CD103+ | TIL | CD8+_CD103+ | TGTGCCAGCAGCTTAGCGGTACTGGGGCGGCGTGGAAACACCATATATTTT |
| 1034 | 172-TL720-TIL-CD8+_CD103+ | TIL | CD8+_CD103+ | TGTGCCAGCAGCTCCGGGAGTCCGTCCGGGGAGCTGTTTTTT |
| 1035 | 172-TL720-TIL-CD8+_CD103+ | TIL | CD8+_CD103+ | TGCGCCAGCAGCCAAGATAGGAAGAGGACAGGGCCCTTGAACACTGAAGC |
| | | | | TTTCTTT |
| 1036 | 172-TL720-TIL-CD8+_CD103+ | TIL | CD8+_CD103+ | TGTGCCAGCAGCTTTTTGCAGTCCTACAATGAGCAGTTCTTC |
| 1037 | 172-TL720-TIL-CD8+_CD103+ | TIL | CD8+_CD103+ | TGTGCCACCAGTGAATCTACCCTTTCTGGCACGGACACAGAT |
| | | | | ACGCAGTATTTT |
| 1038 | 172-TL720-TIL-CD8+_CD103+ | TIL | CD8+_CD103+ | TGTGCCAGCAGTCTCCAGAGGGTGGGCACTGAAGCTTTCTTT |
| 1039 | 172-TL720-TIL-CD8+_CD103+ | TIL | CD8+_CD103+ | TGTGCCAGCAGCTCCCGGACGGGCTCCTACGAGCAGTACTTC |
| 1040 | 172-TL720-TIL-CD8+_CD103+ | TIL | CD8+_CD103+ | TGTGCCATCCAACCAAGATTTAACTTATTCGCTAACTATGGCTACACCTTC |
| 1041 | 172-TL720-TIL-CD8+_CD103+ | TIL | CD8+_CD103+ | TGTGCCAGCAGCCCCGGGACAGTATACACCGGGGAGCTGTTTTTT |
| 1042 | 172-TL720-TIL-CD8+_CD103+ | TIL | CD8+_CD103+ | TGTGCCAGCAGTCCCCCGGGCGGGGGAGAGCAGTACTTC |
| 1043 | 172-TL720-TIL-CD8+_CD103+ | TIL | CD8+_CD103+ | TGTGCCAGCAGTTTGGGCTATGGCTACACCTTC |
| 1044 | 172-TL720-TIL-CD8+_CD103+ | TIL | CD8+_CD103+ | TGTGCCAGCAGCCAAGAATGGACAGGGGGCAGAGATACGCAGTATTTT |
| 1045 | 172-TL720-TIL-CD8+_CD103+ | TIL | CD8+_CD103+ | TGCGCCAGCAGCTTGGCCATTGGGGGAGATGGCTACACCTTC |
| 1046 | 172-TL720-TIL-CD8+_CD103+ | TIL | CD8+_CD103+ | TGTGCCAGCCAAGAGGGCTTAAGCTACGAGCAGTACTTC |
| 1047 | 172-TL720-TIL-CD8+_CD103+ | TIL | CD8+_CD103+ | TGCGCCAGCAGTCCCGGACAGGGGGCGACAGATACGCAGTATTTT |
| 1048 | 172-TL720-TIL-CD8+_CD103+ | TIL | CD8+_CD103+ | TGCGCCAGCAGCCAAGGGACAGGGAACTACGAGCAGTACTTC |
| 1049 | 172-TL720-TIL-CD8+_CD103+ | TIL | CD8+_CD103+ | TGTGCCAGCAGCCCTTATCTTGGCTCCTACAATGAGCAGTTCTTC |
| 1050 | 172-TL720-TIL-CD8+_CD103+ | TIL | CD8+_CD103+ | TGTGCCAGCAGTTACGTGGGGGCAGGCCCGCAGTATTTT |
| 1051 | 172-TL720-TIL-CD8+_CD103+ | TIL | CD8+_CD103+ | TGTGCCAGCAGTGAGTCAACGCTCCACAATGAGCAGTTCTTC |
| 1052 | 172-TL720-TIL-CD8+_CD103+ | TIL | CD8+_CD103+ | TGTGCCAGTAGTATAGCCGGGCTAGCGGGGGGCCTTAATGAGCAGTTCTTC |
| 1053 | 173-TL720-TIL-CD8+_CD103− | TIL | CD8+_CD103− | TGTGCCAGCTCTGACTCCCGGGAGGGGGAGCGAGGAACTGAAGCTTTCTTT |
| 1054 | 173-TL720-TIL-CD8+_CD103− | TIL | CD8+_CD103− | TGCGCCAGCAGCCCGATAGGGACAGGGGAAAACATTCAGTACTTC |
| 1055 | 173-TL720-TIL-CD8+_CD103− | TIL | CD8+_CD103− | TGTGCCAGTAGTATAGACGGCACTTCCTACGAGCAGTACTTC |
| 1056 | 173-TL720-TIL-CD8+_CD103− | TIL | CD8+_CD103− | TGTGCCAGCAGTGAAGGGCAACTTGCACCCGGGGAGCTGTTTTTT |
| 1057 | 173-TL720-TIL-CD8+_CD103− | TIL | CD8+_CD103− | TGTGCCAGCGCCGGGACTAGCGAGTCCAATGAGCAGTTCTTC |
| 1058 | 173-TL720-TIL-CD8+_CD103− | TIL | CD8+_CD103− | TGTGCCAGCAGTTACGTGGGGGCAGGCCCGCAGTATTTT |
| 1059 | 173-TL720-TIL-CD8+_CD103− | TIL | CD8+_CD103− | TGTGCCAGCAGCCCCGGGACAGTATACACCGGGGAGCTGTTTTTT |
| 1060 | 173-TL720-TIL-CD8+_CD103− | TIL | CD8+_CD103− | TGTGCCAGCAGCCCCTTTACTAGCGGGAACACCGGGGAGCTGTTTTTT |
| 1061 | 173-TL720-TIL-CD8+_CD103− | TIL | CD8+_CD103− | TGCAGTGCTTCGGGCCCCGTGGAGGACATTCAGTACTTC |
| 1062 | 173-TL720-TIL-CD8+_CD103− | TIL | CD8+_CD103− | TGCGCCAGCAGTCTTGAGGCCAACGTCCTGACTTTC |
| 1063 | 173-TL720-TIL-CD8+_CD103− | TIL | CD8+_CD103− | TGCAGCGTTACGGGACAGCTGAACACTGAAGCTTTCTTT |
| 1064 | 173-TL720-TIL-CD8+_CD103− | TIL | CD8+_CD103− | TGTGCCAGCAGTGAGTCAACGCTCCACAATGAGCAGTTCTTC |
| 1065 | 173-TL720-TIL-CD8+_CD103− | TIL | CD8+_CD103− | TGTGCCAGCAGCTTGACGGGGACCGGAGTGGAGCAGTACTTC |
| 1066 | 173-TL720-TIL-CD8+_CD103− | TIL | CD8+_CD103− | TGTGCCAGTAGTATGGGGTGGAGTCAGCCCCAGCATTTT |
| 1067 | 173-TL720-TIL-CD8+_CD103− | TIL | CD8+_CD103− | TGCGCCAGCAGCGACGAGCGGGATACGCAGTATTTT |
| 1068 | 173-TL720-TIL-CD8+_CD103− | TIL | CD8+_CD103− | TGTGCCAGCAGTGTGCTGGACAATGAGCAGTTCTTC |
| 1069 | 173-TL720-TIL-CD8+_CD103− | TIL | CD8+_CD103− | TGTGCCATCCAGTCGGGGGGTTCGAACACCGGGGAGCTGTTTTTT |
| 1070 | 173-TL720-TIL-CD8+_CD103− | TIL | CD8+_CD103− | TGTGCCAGCAGCGCCTGGGGTTCGCGCGGCTACACCTTC |
| 1071 | 173-TL720-TIL-CD8+_CD103− | TIL | CD8+_CD103− | TGTGCCAGCAGCCTGGGGACAGGGGACGGCTACACCTTC |
| 1072 | 173-TL720-TIL-CD8+_CD103− | TIL | CD8+_CD103− | TGTGCCAGCAGTTACGGGACAGCCAACAATGAGCAGTTCTTC |
| 1073 | 173-TL720-TIL-CD8+_CD103− | TIL | CD8+_CD103− | TGTGCCAGCCAAGAGGGCTTAAGCTACGAGCAGTACTTC |
| 1074 | 173-TL720-TIL-CD8+_CD103− | TIL | CD8+_CD103− | TGTGCCAGCAATAGAGACACCGCCTCAAATGAGCAGTTCTTC |
| 1075 | 173-TL720-TIL-CD8+_CD103− | TIL | CD8+_CD103− | TGTGCCAGCAGCTTGTCCTCGCCGGGGGCTCCCTACGAGCAGTACTTC |
| 1076 | 173-TL720-TIL-CD8+_CD103− | TIL | CD8+_CD103− | TGTGCCAGCAGCTTAACGGGAGGGACAGGGGGCACTGAAGCTTTCTTT |
| 1077 | 173-TL720-TIL-CD8+_CD103− | TIL | CD8+_CD103− | TGTGCCAGCAGCACCACAGATACGCAGTATTTT |
| 1078 | 173-TL720-TIL-CD8+_CD103− | TIL | CD8+_CD103− | TGTGCCAGCAGTAATAGGGGGAGCGAGCAGTACTTC |
| 1079 | 173-TL720-TIL-CD8+_CD103− | TIL | CD8+_CD103− | TGTGCCAGCAGCCCCTCCCCAGCGGGGGTCCACAATGAGCAGTTCTTC |
| 1080 | 173-TL720-TIL-CD8+_CD103− | TIL | CD8+_CD103− | TGTGCCAGCAGTTCTCCCCTAACAGTTCTCACCGGGGAGCTGTTTTTT |
| 1081 | 173-TL720-TIL-CD8+_CD103− | TIL | CD8+_CD103− | TGTGCCAGCAGCGTTGGGGGGCTGGCCACTGAAGCCTTCTTT |
| 1082 | 173-TL720-TIL-CD8+_CD103− | TIL | CD8+_CD103− | TGTGCCAGCAGCTTTGGCGGGGGGGCCAATGAGCAGTTCTTC |
| 1083 | 173-TL720-TIL-CD8+_CD103− | TIL | CD8+_CD103− | TGTGCCAGCAGCCTACCGCCGAGCGCCGGCTACAATGAGCAGTTCTTC |
| 1084 | 173-TL720-TIL-CD8+_CD103− | TIL | CD8+_CD103− | TGTGCCAGCAGCTTGTTAACAGGGTCTAACAATGAGCAGTTCTTC |
| 1085 | 173-TL720-TIL-CD8+_CD103− | TIL | CD8+_CD103− | TGTGCCAGCAGCTTCGGCTTTCCGGATACGCAGTATTTT |
| 1086 | 173-TL720-TIL-CD8+_CD103− | TIL | CD8+_CD103− | TGTGCCGCCGATGTGCCCGAAACCTCACGGGACAGGGTC |
| | | | | CGTAATGAGCAGTTCTTC |
| 1087 | 173-TL720-TIL-CD8+_CD103− | TIL | CD8+_CD103− | TGTGCCACCAGTGATGGCTCCGGGACAGCCCCCTACGAGCAGTACTTC |
| 1088 | 173-TL720-TIL-CD8+_CD103− | TIL | CD8+_CD103− | TGTGCCAGCAGCTTAGCAGGCGGAGAGCAGTACTTC |
| 1089 | 173-TL720-TIL-CD8+_CD103− | TIL | CD8+_CD103− | TGTGCCAGCAGCTTAGATACAGGCCTCTCTGGAAACACCATATATTTT |
| 1090 | 173-TL720-TIL-CD8+_CD103− | TIL | CD8+_CD103− | TGTGCCAGCAGTCGGGACTCTTCCTACAATGAGCAGTTCTTC |
| 1091 | 173-TL720-TIL-CD8+_CD103− | TIL | CD8+_CD103− | TGTGCCAGCAGTTTGGCGGGGGAGCAGTTCTTC |
| 1092 | 173-TL720-TIL-CD8+_CD103− | TIL | CD8+_CD103− | TGTGCCAGCAGCTCGGACCCCTACGGAGTGATATACGAGCAGTACTTC |
| 1093 | 173-TL720-TIL-CD8+_CD103− | TIL | CD8+_CD103− | TGTGCCAGCAGCCAAGATATCAGCGGGGGCCCCTCCTCCTTC |
| 1094 | 173-TL720-TIL-CD8+_CD103− | TIL | CD8+_CD103− | TGTGCCAGCAGCTTTGACAGTTCGGAAGCTTTCTTT |
| 1095 | 173-TL720-TIL-CD8+_CD103− | TIL | CD8+_CD103− | TGTGCCATCAGTGAGAGGGACAGCGGGACTAACTATGGCTACACCTTC |
| 1096 | 173-TL720-TIL-CD8+_CD103− | TIL | CD8+_CD103− | TGCAGCGTCACCCCGGGTAGTACAGATACGCAGTATTTT |
| 1097 | 173-TL720-TIL-CD8+_CD103− | TIL | CD8+_CD103− | TGTGCCAGCAGTTACTCGAGGACAGGGTTTGTCACTGAAGCTTTCTTT |
| 1098 | 173-TL720-TIL-CD8+_CD103− | TIL | CD8+_CD103− | TGCAGCGTCGCACGTGGAGCCAAGGGTGGCTACACCTTC |
| 1099 | 173-TL720-TIL-CD8+_CD103− | TIL | CD8+_CD103− | TGTGCCAGCAGCTTCCCCCTCGGAGGGCAGTCCTACGAGCAGTACTTC |
| 1100 | 173-TL720-TIL-CD8+_CD103− | TIL | CD8+_CD103− | TGTGCCAGTAGTATCGCGATTGGGGCGGAGCAGTACTTC |
| 1101 | 173-TL720-TIL-CD8+_CD103− | TIL | CD8+_CD103− | TGTGCCTGGACTAGGGACAGGGGGGAGCAGTTCTTC |
| 1102 | 173-TL720-TIL-CD8+_CD103− | TIL | CD8+_CD103− | TGTGCCAGCAGCGAGGATTGGCTAGCGGGGGGGCCT |
| | | | | GCAGATACGCAGTATTTT |
| 1103 | 173-TL720-TIL-CD8+_CD103− | TIL | CD8+_CD103− | TGTGCCAGCCATGACAGCTACGAGCAGTACTTC |
| 1104 | 173-TL720-TIL-CD8+_CD103− | TIL | CD8+_CD103− | TGTGCCAGCAGCGTCGACACTAACTACTATGGCTACACCTTC |
| 1105 | 173-TL720-TIL-CD8+_CD103− | TIL | CD8+_CD103− | TGCAGTGCTACGGGCTTAAACCACACTGAAGCTTTCTTT |
| 1106 | 173-TL720-TIL-CD8+_CD103− | TIL | CD8+_CD103− | TGTGCCAGCAGCGCGGGGATCCAAAACACCATATATTTT |
| 1107 | 173-TL720-TIL-CD8+_CD103− | TIL | CD8+_CD103− | TGCGCCAGCAGTGAGGACGGTATGAACACTGAAGCTTTCTTT |
| 1108 | 173-TL720-TIL-CD8+_CD103− | TIL | CD8+_CD103− | TGTGCCAGCAGCTTTGGACGGGTTACGGGGGAGACCCAGTACTTC |
| 1109 | 173-TL720-TIL-CD8+_CD103− | TIL | CD8+_CD103− | TGCAGTGCTAGAGGCCCTTTACCCTCCTTC |
| 1110 | 173-TL720-TIL-CD8+_CD103− | TIL | CD8+_CD103− | TGTGCCAGCAGCAGCCGGGACAGGGACCGTAATGAGCAGTTCTTC |
| 1111 | 173-TL720-TIL-CD8+_CD103− | TIL | CD8+_CD103− | TGTGCCAGCAGTTCTCTGTACAGCGGGATAGAGAGCTCCTACAAT |
| | | | | GAGCAGTTCTTC |
| 1112 | 173-TL720-TIL-CD8+_CD103− | TIL | CD8+_CD103− | TGTGCCAGCAGCTTGGTGGGACAGGGTTCCAATGAGCAGTTCTTC |
| 1113 | 173-TL720-TIL-CD8+_CD103− | TIL | CD8+_CD103− | TGCAGTGCTAGAGATCCCGACCCGCTGGATGGCTACACCTTC |
| 1114 | 173-TL720-TIL-CD8+_CD103− | TIL | CD8+_CD103− | TGTGCCAGCAGTAGCGGGGGGGAAAATGAGCAGTTCTTC |
| 1115 | 173-TL720-TIL-CD8+_CD103− | TIL | CD8+_CD103− | TGCGCCAGCAGCCAAGATCCAGAAATCAACACTGAAGCTTTCTTT |
| 1116 | 173-TL720-TIL-CD8+_CD103− | TIL | CD8+_CD103− | TGTGCCAGCAGTTTTCCAGACAGGGACAATGAAAAACTGTTTTTT |
| 1117 | 173-TL720-TIL-CD8+_CD103− | TIL | CD8+_CD103− | TGTGCCAGCAACCTCTGGGGGGGCTCCTACAATGAGCAGTTCTTC |
| 1118 | 173-TL720-TIL-CD8+_CD103− | TIL | CD8+_CD103− | TGTGCCAGCAGTGACAGCCAATTCACAGATACGCAGTATTTT |
| 1119 | 173-TL720-TIL-CD8+_CD103− | TIL | CD8+_CD103− | TGCGCCAGCAGCCAAGAAAGCGGGAGTTACTACGAGCAGTACTTC |
| 1120 | 173-TL720-TIL-CD8+_CD103− | TIL | CD8+_CD103− | TGCAGTGCTAGGAGACTAGCGGGGACCGACAATGAGCAGTTCTTC |
| 1121 | 173-TL720-TIL-CD8+_CD103− | TIL | CD8+_CD103− | TGTGCCAGCAGCCAAGACTCCCGGTACAATGAGCAGTTCTTC |
| 1122 | 173-TL720-TIL-CD8+_CD103− | TIL | CD8+_CD103− | TGTGCCAGCAGCCTGATCCCCGGGGCCACAGATACGCAGTATTTT |
| 1123 | 173-TL720-TIL-CD8+_CD103− | TIL | CD8+_CD103− | TGTGCCAGCAGCAGGACAGATCCTAACCCTGGAAACACCATATATTTT |
| 1124 | 173-TL720-TIL-CD8+_CD103− | TIL | CD8+_CD103− | TGTGCCAGTAGTATAGCGACACCAGGGGAGCAGTACTTC |
| 1125 | 173-TL720-TIL-CD8+_CD103− | TIL | CD8+_CD103− | TGTGCCAGCAGTTTCCCCGGAGGAGGAGTCGATGAAGCTTTCTTT |
| 1126 | 173-TL720-TIL-CD8+_CD103− | TIL | CD8+_CD103− | TGTGCCAGCAGCCAAGATCTCTGGACAATGAACACCGGGGAGCTGTTTTTT |
| 1127 | 173-TL720-TIL-CD8+_CD103− | TIL | CD8+_CD103− | TGTGCCAGCAGCGTTGGGGGGCTGGCCACTGAAGCTTTCTTT |
| 1128 | 173-TL720-TIL-CD8+_CD103− | TIL | CD8+_CD103− | TGTGCCAGCAGTTTGGGGCTAGGGCGGGGGGGCTTGAACACCGGG |
| | | | | GAGCTGTTTTTT |
| 1129 | 173-TL720-TIL-CD8+_CD103− | TIL | CD8+_CD103− | TGCAGCGTTGACCGGACCGGCGAGACCCAGTACTTC |
| 1130 | 173-TL720-TIL-CD8+_CD103− | TIL | CD8+_CD103− | TGCAGCGCAGGGGGGACTAGCGGGGGGATTGAGCAGTTCTTC |
| 1131 | 173-TL720-TIL-CD8+_CD103− | TIL | CD8+_CD103− | TGTGCCAGCAGTTTAGGTAGCGGGCAAGAGACCCAGTACTTC |
| 1132 | 173-TL720-TIL-CD8+_CD103− | TIL | CD8+_CD103− | TGTGCCAGCAGTTTAGGGGGACAGAACTATGGCTACACCTTC |
| 1133 | 173-TL720-TIL-CD8+_CD103− | TIL | CD8+_CD103− | TGTGCCAGCAGCTTAGGGTTATCCTCCTACGAGCAGTACTTC |
| 1134 | 173-TL720-TIL-CD8+_CD103− | TIL | CD8+_CD103− | TGCAGTGCTAGACCACTAGCGAGGGTAAACTACGAGCAGTACTTC |
| 1135 | 173-TL720-TIL-CD8+_CD103− | TIL | CD8+_CD103− | TGCGCCAGCAGCTGGACAGTTTCGTCGGACTATGGCTACACCTTC |
| 1136 | 173-TL720-TIL-CD8+_CD103− | TIL | CD8+_CD103− | TGTGCCAGCAGCTTATTTCTCTTCGGTAAGCCCCAGCATTTT |
| 1137 | 173-TL720-TIL-CD8+_CD103− | TIL | CD8+_CD103− | TGTGCCAGCAGCTTAGACGGGCCTCTGGATGAGCAGTTCTTC |
| 1138 | 173-TL720-TIL-CD8+_CD103− | TIL | CD8+_CD103− | TGTGCCAGCAGCTACTGGGACAGGGCTTACTTC |
| 1139 | 173-TL720-TIL-CD8+_CD103− | TIL | CD8+_CD103− | TGTGCCAGCAGTCCCCGACAGGGGAAGACCCAGTACTTC |
| 1140 | 173-TL720-TIL-CD8+_CD103− | TIL | CD8+_CD103− | TGTGCCACCCCAGGAGGTGATGGCAATCAGCCCCAGCATTTT |
| 1141 | 173-TL720-TIL-CD8+_CD103− | TIL | CD8+_CD103− | TGTGCCACCAGTGAATCTACCCTTTCTGGCACGGACACAGAT |
| | | | | ACGCAGTATTTT |
| 1142 | 173-TL720-TIL-CD8+_CD103− | TIL | CD8+_CD103− | TGTGCCAGCAGTCTCCAGAGGGTGGGCACTGAAGCTTTCTTT |
| 1143 | 173-TL720-TIL-CD8+_CD103− | TIL | CD8+_CD103− | TGTGCCAGTAGTTCACCGACAGGGGGCCCCTACAATGAGCAGTTCTTC |
| 1144 | 173-TL720-TIL-CD8+_CD103− | TIL | CD8+_CD103− | TGCAGCGTTGTGCCTCCCGGGGGCCCAGATACGCAGTATTTT |
| 1145 | 173-TL720-TIL-CD8+_CD103− | TIL | CD8+_CD103− | TGTGCCAGCAGCCAAGAATGGACAGGGGGCAGAGATACGCAGTATTTT |
| 1146 | 173-TL720-TIL-CD8+_CD103− | TIL | CD8+_CD103− | TGTGCCAGCAGCCAAGTAGCCAATGAGCAGTTCTTC |
| 1147 | 173-TL720-TIL-CD8+_CD103− | TIL | CD8+_CD103− | TGTGCCAGCAGCTTGCCGCCCGTGGACCGGGGAGAGACCCAGTACTTC |
| 1148 | 173-TL720-TIL-CD8+_CD103− | TIL | CD8+_CD103− | TGTGCCAGCAGCTTGCTCCCCGGACATATAGAAACAGATACGCAGTATTTT |
| 1149 | 173-TL720-TIL-CD8+_CD103− | TIL | CD8+_CD103− | TGTGCCAGCAGCCCCGACCTTAACTATGGCTACACCTTC |
| 1150 | 173-TL720-TIL-CD8+_CD103− | TIL | CD8+_CD103− | TGTGCCAGCAGTCCACAAACAGGGAACACCGGGGAGCTGTTTTTT |
| 1151 | 173-TL720-TIL-CD8+_CD103− | TIL | CD8+_CD103− | TGTGCCAGCAGTTTACCAGGGTACCCCGGAAACACCATATATTTT |
| 1152 | 173-TL720-TIL-CD8+_CD103− | TIL | CD8+_CD103− | TGTGCCAGTAGTCCGCGCCGGGACCCTGGGGGTGAGCAGTTCTTC |
| 1153 | 173-TL720-TIL-CD8+_CD103− | TIL | CD8+_CD103− | TGCGCCAGCAGCCGATCCTTGTTGCACAGGGTCATGAACACTGAA |
| | | | | GCTTTCTTT |
| 1154 | 173-TL720-TIL-CD8+_CD103− | TIL | CD8+_CD103− | TGCAGTGCTAGAGATTGGAGTGGCATCCCCGGGGAGCTGTTTTTT |
| 1155 | 173-TL720-TIL-CD8+_CD103− | TIL | CD8+_CD103− | TGTGCCAGCAGCGCCGCTGAACTGACTGGGTGGAGCGGGGGGCCC |
| | | | | AATGAGCAGTTCTTC |
| 1156 | 173-TL720-TIL-CD8+_CD103− | TIL | CD8+_CD103− | TGTGCCAGCAGTCCCACCAAATGGTCTAACTATGGCTACACCTTC |
| 1157 | 173-TL720-TIL-CD8+_CD103− | TIL | CD8+_CD103− | TGTGCCAGCAGCCGACAGGGCGGAAACTATGGCTACACCTTC |
| 1158 | 173-TL720-TIL-CD8+_CD103− | TIL | CD8+_CD103− | TGTGCCAGCAGCCCGCAGCGGGGGGGGACCGGGAATGAGCAGTTCTTC |
| 1159 | 173-TL720-TIL-CD8+_CD103− | TIL | CD8+_CD103− | TGTGCCAGCAGCGAGGCCGGTTATAATCAGCCCCAGCATTTT |
| 1160 | 173-TL720-TIL-CD8+_CD103− | TIL | CD8+_CD103− | TGCGCCAGCAGCTCGGGACAGGATAATTCACCCCTCCACTTT |
| 1161 | 173-TL720-TIL-CD8+_CD103− | TIL | CD8+_CD103− | TGTGCCAGCAGCTTGGCGAGACAGGGAAAGGAGACCCAGTACTTC |
| 1162 | 173-TL720-TIL-CD8+_CD103− | TIL | CD8+_CD103− | TGCGCCAGCAGCCAACAAAACACCGGGGAGCTGTTTTTT |
| 1163 | 173-TL720-TIL-CD8+_CD103− | TIL | CD8+_CD103− | TGTGCCAGCAGCTGGGAACTGACAGGCCCTCCCTACGAGCAGTACTTC |
| 1164 | 173-TL720-TIL-CD8+_CD103− | TIL | CD8+_CD103− | TGTGCCAGCAGCCAAGGGACAGGGGTGAATGAGCAGTTCTTC |
| 1165 | 173-TL720-TIL-CD8+_CD103− | TIL | CD8+_CD103− | TGCGCCAGCAGCCAAGGGCAGTTCTATGGCTACACCTTC |
| 1166 | 173-TL720-TIL-CD8+_CD103− | TIL | CD8+_CD103− | TGTGCCAGCAGCAACGGGACAGGCCTCCGGGCTGGGGGCTACACCTTC |
| 1167 | 173-TL720-TIL-CD8+_CD103− | TIL | CD8+_CD103− | TGCGCCAGCAGCCAAGATCCCGACATCGGGGAGCTGTTTTTT |
| 1168 | 173-TL720-TIL-CD8+_CD103− | TIL | CD8+_CD103− | TGTGCCAGCAGTCCCCCCGATACCTACAATGAGCAGTTCTTC |
| 1169 | 173-TL720-TIL-CD8+_CD103− | TIL | CD8+_CD103− | TGTGCCAGCAGCCAAGTTGGATCCAATGAGCAGTTCTTC |
| 1170 | 173-TL720-TIL-CD8+_CD103− | TIL | CD8+_CD103− | TGTGCCAGCAGCTTAATGGTCCACACTGAAGCTTTCTTT |
| 1171 | 173-TL720-TIL-CD8+_CD103− | TIL | CD8+_CD103− | TGTGCCAGCAGCTTAGCGATAGATACGCAGTATTTT |
| 1172 | 173-TL720-TIL-CD8+_CD103− | TIL | CD8+_CD103− | TGTGCCAGCAGTTTAGCTCCTGAGGCTAGCGGATACAATGAGCAGTTCTTC |
| 1173 | 173-TL720-TIL-CD8+_CD103− | TIL | CD8+_CD103− | TGTGCCAGCAGTGGGGAGACCCTACACACTGAAGCTTTCTTT |
| 1174 | 173-TL720-TIL-CD8+_CD103− | TIL | CD8+_CD103− | TGTGCCACCAGCAAACTAGCGGGGGGGGGAGATACGCAGTATTTT |
| 1175 | 173-TL720-TIL-CD8+_CD103− | TIL | CD8+_CD103− | TGTGCCAGCAGTTATGGACAGAACTATGGCTACACCTTC |
| 1176 | 173-TL720-TIL-CD8+_CD103− | TIL | CD8+_CD103− | TGTGCCATCAGTGAGGGTGTTAGCACAGATACGCAGTATTTT |
| 1177 | 173-TL720-TIL-CD8+_CD103− | TIL | CD8+_CD103− | TGCGCCAGCAGCCAAGATCTAAGGGGCAATGAGCAGTTCTTC |
| 1178 | 173-TL720-TIL-CD8+_CD103− | TIL | CD8+_CD103− | TGTGCCAGCAGCGAGGGGGGACAGCTGAATGAAAAACTGTTTTTT |
| 1179 | 173-TL720-TIL-CD8+_CD103− | TIL | CD8+_CD103− | TGTGCCAGCAGCCCGACAGCCTACGAGCAGTACTTC |
| 1180 | 173-TL720-TIL-CD8+_CD103− | TIL | CD8+_CD103− | TGTGCCAGTTCAGGGGACGAGGGTCAGCCCCAGCATTTT |
| 1181 | 173-TL720-TIL-CD8+_CD103− | TIL | CD8+_CD103− | TGTGCCAGCTCACCCTTGGACGACCCCACCGGGGAGCTGTTTTTT |
| 1182 | 173-TL720-TIL-CD8+_CD103− | TIL | CD8+_CD103− | TGTGCCAGCAGCTTACACCAGGGTCCCTCCTACGAGCAGTACTTC |
| 1183 | 173-TL720-TIL-CD8+_CD103− | TIL | CD8+_CD103− | TGTGCCAGCAGCCCTCCGGGTAGGGAGAACACCGGGGAGCTGTTTTTT |
| 1184 | 173-TL720-TIL-CD8+_CD103− | TIL | CD8+_CD103− | TGCAGTGCTACAACACCAAACTATGGCTACACCTTC |
| 1185 | 173-TL720-TIL-CD8+_CD103− | TIL | CD8+_CD103− | TGTGCCAGCACCAACATACACAGCACAGATACGCAGTATTTT |
| 1186 | 173-TL720-TIL-CD8+_CD103− | TIL | CD8+_CD103− | TGTGCCAGCAGAAAGACTGGGGTCTCCACTGAAGCTTTCTTT |
| 1187 | 173-TL720-TIL-CD8+_CD103− | TIL | CD8+_CD103− | TGTGCCAGCAGCCAAGAATCMCTTTTCCCTGGACACCGGGGAGCTGTTTTTT |
| 1188 | 173-TL720-TIL-CD8+_CD103− | TIL | CD8+_CD103− | TGTGCCAGCAGTGAAGGGGGACAGAACTATGGCTACACCTTC |
| 1189 | 173-TL720-TIL-CD8+_CD103− | TIL | CD8+_CD103− | TGTGCCTGGAGGGGCAGGGGGCTGAGTGAGCAGTTCTTC |
| 1190 | 173-TL720-TIL-CD8+_CD103− | TIL | CD8+_CD103− | TGTGCCAGCAGCTTGTATGCAGGTCCTAACTATGGCTACACCTTC |
| 1191 | 173-TL720-TIL-CD8+_CD103− | TIL | CD8+_CD103− | TGTGCCAGCAGCCCTCTGACTAGCGGGGATGAGCAGTTCTTC |
| 1192 | 173-TL720-TIL-CD8+_CD103− | TIL | CD8+_CD103− | TGTGCCAGCAGCTTCGGGACAGGGACCCCAGATACGCAGTATTTT |
| 1193 | 173-TL720-TIL-CD8+_CD103− | TIL | CD8+_CD103− | TGTGCCAGCAGTCGCCCATACTCCAAAGAGACCCAGTACTTC |
| 1194 | 173-TL720-TIL-CD8+_CD103− | TIL | CD8+_CD103− | TGTGCCTGGTACTCCTCTGGGGCCAACGTCCTGACTTTC |
| 1195 | 173-TL720-TIL-CD8+_CD103− | TIL | CD8+_CD103− | TGCGCCAGCAGCTTGTTGGGCGGGGAGATCTACAATGAGCAGTTCTTC |
| 1196 | 173-TL720-TIL-CD8+_CD103− | TIL | CD8+_CD103− | TGCGCCAGCAGCCGGACTAGCGGGGGGGCGGATGAGCAGTTCTTC |
| 1197 | 173-TL720-TIL-CD8+_CD103− | TIL | CD8+_CD103− | TGTGCCAGCAGCCCCGGGACTAGCGGCGACAATGAGCAGTTCTTC |
| 1198 | 173-TL720-TIL-CD8+_CD103− | TIL | CD8+_CD103− | TGTGCCAGCAGCAACGACATGACACCTGGGTGGATCTTC |
| 1199 | 18-TL615-TIL-CD8+_CD103+ | TIL | CD8+_CD103+ | TGTGCCAGCAGTAGGGGACTAGCGGGAGTCAATGAGCAGTTCTTC |
| 1200 | 18-TL615-TIL-CD8+_CD103+ | TIL | CD8+_CD103+ | TGTGCCAGCAGTTTTCCGTCGCGGGAGGAGACCCAGTACTTC |
| 1201 | 18-TL615-TIL-CD8+_CD103+ | TIL | CD8+_CD103+ | TGTGCCAGCAGTTACGGCCCCGAGCGGGCTGAAGCTTTCTTT |
| 1202 | 18-TL615-TIL-CD8+_CD103+ | TIL | CD8+_CD103+ | TGTGCCAGCAGCGATTGGGAGAGCACAGATACGCAGTATTTT |
| 1203 | 18-TL615-TIL-CD8+_CD103+ | TIL | CD8+_CD103+ | TGTGCCAGCAGCTTAATCCGGGTGGGGGGAAATCAGCCCCAGCATTTT |
| 1204 | 18-TL615-TIL-CD8+_CD103+ | TIL | CD8+_CD103+ | TGTGCCAGCAGTGAAGAGGGATACGCCTACGAGCAGTACTTC |
| 1205 | 18-TL615-TIL-CD8+_CD103+ | TIL | CD8+_CD103+ | TGTGCCAGCAGCTTGATCAATGAGCAGTTCTTC |
| 1206 | 18-TL615-TIL-CD8+_CD103+ | TIL | CD8+_CD103+ | TGCAGCGTTAAGCGGGAGGGCACCGGGGAGCTGTTTTTT |
| 1207 | 18-TL615-TIL-CD8+_CD103+ | TIL | CD8+_CD103+ | TGTGCCAGCAGTTTAAGACTCCCCGGGCACACTGAAGCTTTCTTT |
| 1208 | 18-TL615-TIL-CD8+_CD103+ | TIL | CD8+_CD103+ | TGTGCCAGCAGCTTGGCCAGGGGTACCGACGAGCAGTACTTC |
| 1209 | 18-TL615-TIL-CD8+_CD103+ | TIL | CD8+_CD103+ | TGTGCCACCAGCTCCATAGGAGGAGACGAGCAGTACTTC |
| 1210 | 18-TL615-TIL-CD8+_CD103+ | TIL | CD8+_CD103+ | TGTGCCAGCAGTCCTGACAGTATTCAACGGGTGTTC |
| 1211 | 18-TL615-TIL-CD8+_CD103+ | TIL | CD8+_CD103+ | TGTGCCAGCAGTCCCCACAATGAGCAGTTCTTC |
| 1212 | 18-TL615-TIL-CD8+_CD103+ | TIL | CD8+_CD103+ | TGTGCCAGCAGCTCCTTTTCGGGGTTGGGCTACGAGCAGTACTTC |
| 1213 | 18-TL615-TIL-CD8+_CD103+ | TIL | CD8+_CD103+ | TGCGCCAGCAGACTGACAGGGGAAAGGACCTACGAGCAGTACTTC |
| 1214 | 18-TL615-TIL-CD8+_CD103+ | TIL | CD8+_CD103+ | TGCGCCAGCGCCCTCCCCGGGTCCTCCTACGAGCAGTACTTC |
| 1215 | 18-TL615-TIL-CD8+_CD103+ | TIL | CD8+_CD103+ | TGTGCCAGCCGATTTCTTAGCGGGAGTCTTGATGAGCAGTTCTTC |
| 1216 | 18-TL615-TIL-CD8+_CD103+ | TIL | CD8+_CD103+ | TGTGCCATCAGCACCTACAGGGCACCCCCTGGAAACACCATATATTTT |
| 1217 | 18-TL615-TIL-CD8+_CD103+ | TIL | CD8+_CD103+ | TGTGCCAGCAGCTTAATCTCTGAAGCTTTCTTT |
| 1218 | 18-TL615-TIL-CD8+_CD103+ | TIL | CD8+_CD103+ | TGCGCCAGCAGCCCCCGACAGGGGGTGGACACTGAAGCTTTCTTT |
| 1219 | 18-TL615-TIL-CD8+_CD103+ | TIL | CD8+_CD103+ | TGTGCCAGCAGCCCGACATTTGAAGCTTTCTTT |
| 1220 | 18-TL615-TIL-CD8+_CD103+ | TIL | CD8+_CD103+ | TGTGCCAGCAGTCCCGACACGGCCCAGCATTTT |
| 1221 | 18-TL615-TIL-CD8+_CD103+ | TIL | CD8+_CD103+ | TGTGCCAGTAGTATAGGCTGGGGTAGCTCCTACAATGAGCAGTTCTTC |
| 1222 | 18-TL615-TIL-CD8+_CD103+ | TIL | CD8+_CD103+ | TGTGCCAGCAGTGAGGGACAACTAGCTCCCGGGGAGCTGTTTTTT |
| 1223 | 18-TL615-TIL-CD8+_CD103+ | TIL | CD8+_CD103+ | TGTGCCAGCAGGGAGGGGATGGCTGAAGCTTTCTTT |
| 1224 | 18-TL615-TIL-CD8+_CD103+ | TIL | CD8+_CD103+ | TGCGCCAGCAAGACGGACATGAACACTGAAGCTTTCTTT |
| 1225 | 18-TL615-TIL-CD8+_CD103+ | TIL | CD8+_CD103+ | TGTGCCAGCAGTTTTGAGGGGCGACTTACTGAAGCTTTCTTT |
| 1226 | 18-TL615-TIL-CD8+_CD103+ | TIL | CD8+_CD103+ | TGTGCCAGCAGCCCACCCAACCGGAGACTGTTTTTT |
| 1227 | 18-TL615-TIL-CD8+_CD103+ | TIL | CD8+_CD103+ | TGTGCCAGCAGCACCAGCCCAGGGGGCCGGGGCTACACCTTC |
| 1228 | 18-TL615-TIL-CD8+_CD103+ | TIL | CD8+_CD103+ | TGCAGCGTTGAAGGCCGGACCTCCAGCTCCTACAATGAGCAGTTCTTC |
| 1229 | 18-TL615-TIL-CD8+_CD103+ | TIL | CD8+_CD103+ | TGTGCCAGCAGCTTGGGGGGGAACACTGAAGCTTTCTTT |
| 1230 | 18-TL615-TIL-CD8+_CD103+ | TIL | CD8+_CD103+ | TGTGCCAGTAGTTACGGGACAGGGGGCGGCTATGGCTACACCTTC |
| 1231 | 18-TL615-TIL-CD8+_CD103+ | TIL | CD8+_CD103+ | TGTGCCAGCACCTTAGAGTTTTCCTACGAGCAGTACTTC |
| 1232 | 18-TL615-TIL-CD8+_CD103+ | TIL | CD8+_CD103+ | TGTGCCAGCAGCTTAACTAGCGGGCCATACAATGAGCAGTTCTTC |
| 1233 | 18-TL615-TIL-CD8+_CD103+ | TIL | CD8+_CD103+ | TGTGCCAGCAGTTTCGTCGCGGGCACAGATACGCAGTATTTT |
| 1234 | 18-TL615-TIL-CD8+_CD103+ | TIL | CD8+_CD103+ | TGTGCCAGCAGCTTAGGTCGAAACGAGCAGTACTTC |
| 1235 | 18-TL615-TIL-CD8+_CD103+ | TIL | CD8+_CD103+ | TGTGCCAGCAGCGTAACGGGGACAGGGACCAAAAACATTCAGTACTTC |
| 1236 | 18-TL615-TIL-CD8+_CD103+ | TIL | CD8+_CD103+ | TGCGCCAGCAGCCAAGTCCAAGGGGGTTCCTACGAGCAGTACTTC |
| 1237 | 18-TL615-TIL-CD8+_CD103+ | TIL | CD8+_CD103+ | TGTGCCATCAGTGAATCCCAAGCAGGTTCCTACAATGAGCAGTTCTTC |
| 1238 | 18-TL615-TIL-CD8+_CD103+ | TIL | CD8+_CD103+ | TGTGCCAGCAGTTTAAGTCTAGCTGGGGATGAGCAGTTCTTC |
| 1239 | 18-TL615-TIL-CD8+_CD103+ | TIL | CD8+_CD103+ | TGTGCCAGCAGCCCGACGGACAGGGGCGCCAGCACTGAAGCTTTCTTT |
| 1240 | 18-TL615-TIL-CD8+_CD103+ | TIL | CD8+_CD103+ | TGTGCCAGCAGTTTGGGTCCGGGGACTAGCGTCTACGAGCAGTACTTC |
| 1241 | 18-TL615-TIL-CD8+_CD103+ | TIL | CD8+_CD103+ | TGTGCCAGCAGTTACGGGGCAGGGGACTACGAGCAGTACTTC |
| 1242 | 18-TL615-TIL-CD8+_CD103+ | TIL | CD8+_CD103+ | TGTGCCACCAGCCCCGGGACGGAGGGCGAGCAGTACTTC |
| 1243 | 18-TL615-TIL-CD8+_CD103+ | TIL | CD8+_CD103+ | TGTGCCAGCAGTGAGCCTGTTACGGGACTAGCGGGGCGG |
| | | | | AGCTCCTACAATGAGCAGTTCTTC |
| 1244 | 18-TL615-TIL-CD8+_CD103+ | TIL | CD8+_CD103+ | TGTGCCAGCAGTCCGGACAGGATGAACACTGAAGCTTTCTTT |
| 1245 | 18-TL615-TIL-CD8+_CD103+ | TIL | CD8+_CD103+ | TGCGCCAGCAGCCAAGAAGCCGACCAGGGGGTATACAATGAGCAGTTCTTC |
| 1246 | 18-TL615-TIL-CD8+_CD103+ | TIL | CD8+_CD103+ | TGTGCCACCAGTGATAAGAGGGACGAGCAGTACTTC |
| 1247 | 18-TL615-TIL-CD8+_CD103+ | TIL | CD8+_CD103+ | TGTGCCAGCAGTTTAATTGCACTAGGTATGGAAGATACGCAGTATTTT |
| 1248 | 18-TL615-TIL-CD8+_CD103+ | TIL | CD8+_CD103+ | TGTGCCTGGAGTGGTGACAGCAGTAACACTGAAGCTTTCTTT |
| 1249 | 18-TL615-TIL-CD8+_CD103+ | TIL | CD8+_CD103+ | TGTGCCAGCAGTTTTGGAGGGGACGGCTACGAGCAGTACTTC |
| 1250 | 18-TL615-TIL-CD8+_CD103+ | TIL | CD8+_CD103+ | TGTGCCAGCAGTCCCCCAGCGGGACTCACAGATACGCAGTATTTT |
| 1251 | 18-TL615-TIL-CD8+_CD103+ | TIL | CD8+_CD103+ | TGTGCCAGCAGTCCACTGACCCTCTTCCTAGCGGGGCCCTACAAT |
| | | | | GAGCAGTTCTTC |
| 1252 | 18-TL615-TIL-CD8+_CD103+ | TIL | CD8+_CD103+ | TGCGCCAGCAGCCATGACAGGTCAACTAATGAAAAACTGTTTTTT |
| 1253 | 18-TL615-TIL-CD8+_CD103+ | TIL | CD8+_CD103+ | TGTGCCAGCAGTTGGGAGGGCCCCTACGAGCAGTACTTC |
| 1254 | 18-TL615-TIL-CD8+_CD103+ | TIL | CD8+_CD103+ | TGTGCCAGCAGGGACAGGGTCGGGGAGCTGTTTTTT |
| 1255 | 18-TL615-TIL-CD8+_CD103+ | TIL | CD8+_CD103+ | TGTGCCAGTAGTATAGGACTGTCTAGTCTCAATGAGCAGTTCTTC |
| 1256 | 18-TL615-TIL-CD8+_CD103+ | TIL | CD8+_CD103+ | TGTGCCAGCAGTTACTTTGGGGACACTGAAGCTTTCTTT |
| 1257 | 18-TL615-TIL-CD8+_CD103+ | TIL | CD8+_CD103+ | TGTGCCAGCAGCTTCAAGGGAGAGAATGAAAAACTGTTTTTT |
| 1258 | 18-TL615-TIL-CD8+_CD103+ | TIL | CD8+_CD103+ | TGCGCCAGCAGCCAAGGGACAGGTTGGGGAGGCACTGAAGCTTTCTTT |
| 1259 | 18-TL615-TIL-CD8+_CD103+ | TIL | CD8+_CD103+ | TGTGCCAGCAGCCAAGACCCAGGGGGGACAGGGCTTTTGGAAAAACTG |
| | | | | TTTTTT |
| 1260 | 18-TL615-TIL-CD8+_CD103+ | TIL | CD8+_CD103+ | TGTGCCAGCTCAGGCCGGGACAGGGGTTACGAGCAGTACTTC |
| 1261 | 18-TL615-TIL-CD8+_CD103+ | TIL | CD8+_CD103+ | TGTGCCAGCAGCGCTCCGGGGGGGACAGGGGTGGTCGAGACCCAGTACTTC |
| 1262 | 18-TL615-TIL-CD8+_CD103+ | TIL | CD8+_CD103+ | TGCGCCAGCAGCCAGACTGGGACAGGGAACACTGAAGCTTTCTTT |
| 1263 | 18-TL615-TIL-CD8+_CD103+ | TIL | CD8+_CD103+ | TGTGCCATCAGTGATGTAGATACTGGAAACACCATATATTTT |
| 1264 | 18-TL615-TIL-CD8+_CD103+ | TIL | CD8+_CD103+ | TGCGCCAGCAGTTTAGGACCGAACACCGGGGAGCTGTTTTTT |
| 1265 | 18-TL615-TIL-CD8+_CD103+ | TIL | CD8+_CD103+ | TGTGCCAGCTCACCCTCATCTAGCGGGAATTGGGATGAGCAGTTCTTC |
| 1266 | 18-TL615-TIL-CD8+_CD103+ | TIL | CD8+_CD103+ | TGTGCCAGCAGCTTAAGGGGACAGGGTTTGAGGGGGGTCGCGGCTTTCTTT |
| 1267 | 18-TL615-TIL-CD8+_CD103+ | TIL | CD8+_CD103+ | TGCAGTGCTAGACTAGCGGGAGCCTTAAGGTTCGAGCAGTTCTTC |
| 1268 | 18-TL615-TIL-CD8+_CD103+ | TIL | CD8+_CD103+ | TGCGCCAGCAGCTTGGCAGGAGCTCTCTCCTACGAGCAGTACTTC |
| 1269 | 18-TL615-TIL-CD8+_CD103+ | TIL | CD8+_CD103+ | TGTGCCAGCAGTCCCCACCGCTACGAGCAGTACTTC |
| 1270 | 18-TL615-TIL-CD8+_CD103+ | TIL | CD8+_CD103+ | TGCAGCGCCACATTCCCACAGGGGGCACCTTACAATGAGCAGTTCTTC |
| 1271 | 18-TL615-TIL-CD8+_CD103+ | TIL | CD8+_CD103+ | TGTGCCAGCAGCTTAGGGGAGCGACTAGCGGTTACCTACAATGAGCAG |
| | | | | TTCTTC |
| 1272 | 18-TL615-TIL-CD8+_CD103+ | TIL | CD8+_CD103+ | TGCAGTGCTCCTTACGAGACGTGGGCGAAGATCGAGAACACTGAAGCT |
| | | | | TTCTTT |
| 1273 | 18-TL615-TIL-CD8+_CD103+ | TIL | CD8+_CD103+ | TGTGCCAGCAGCGTAGGGACAGATTACGAGCAGTACTTC |
| 1274 | 18-TL615-TIL-CD8+_CD103+ | TIL | CD8+_CD103+ | TGCGCCAGCAGCCACGTCGGGACTAGCGGTTACGAGCAGTACTTC |
| 1275 | 18-TL615-TIL-CD8+_CD103+ | TIL | CD8+_CD103+ | TGCAGCGTTGTGACAGAGAACACTGAAGCTTTCTTT |
| 1276 | 18-TL615-TIL-CD8+_CD103+ | TIL | CD8+_CD103+ | TGTGCCTCGGGGTATCAACATTCGAACACTGAAGCTTTCTTT |
| 1277 | 18-TL615-TIL-CD8+_CD103+ | TIL | CD8+_CD103+ | TGTGCCACCATTCGGACCGGGGGTGCTGGCAATCAGCCCCAGCATTTT |
| 1278 | 18-TL615-TIL-CD8+_CD103+ | TIL | CD8+_CD103+ | TGTGCCATCAGTGGAACTAGCCCCTACAATGAGCAGTTCTTC |
| 1279 | 18-TL615-TIL-CD8+_CD103+ | TIL | CD8+_CD103+ | TGTGCCAGCCCCCGACTAGCGGGAGGGGAGACCGGGGAGCTGTTTTTT |
| 1280 | 18-TL615-TIL-CD8+_CD103+ | TIL | CD8+_CD103+ | TGCAGCGTTGCTGACAATGAGCAGTTCTTC |
| 1281 | 18-TL615-TIL-CD8+_CD103+ | TIL | CD8+_CD103+ | TGCGCCAGCAGCCAAGGAGCGGGAGCCCCCGTTGAGCAGTTCTTC |
| 1282 | 18-TL615-TIL-CD8+_CD103+ | TIL | CD8+_CD103+ | TGTGCCAGCAGTTTTCGGCCCCCTCCCTACGAGCAGTACTTC |
| 1283 | 18-TL615-TIL-CD8+_CD103+ | TIL | CD8+_CD103+ | TGTGCCAGTACAGATACGCGAGATGGCTACACCTTC |
| 1284 | 18-TL615-TIL-CD8+_CD103+ | TIL | CD8+_CD103+ | TGCAGTGCTACTCCTGCTAGCGGGAGGGAGTACAATGAGCAGTTCTTC |
| 1285 | 18-TL615-TIL-CD8+_CD103+ | TIL | CD8+_CD103+ | TGTGCCAGCAGCTTACAGGGGGCGAGGGCGACCGAGCAGTACTTC |
| 1286 | 18-TL615-TIL-CD8+_CD103+ | TIL | CD8+_CD103+ | TGTGCCACCAGCAGAGATTGGGGGAGCTCCTACAATGAGCAGTTCTTC |
| 1287 | 18-TL615-TIL-CD8+_CD103+ | TIL | CD8+_CD103+ | TGTGCCAGCAGCCCCGTAGCGGGAGGGTTGTTGTATGAGCAGTTCTTC |
| 1288 | 18-TL615-TIL-CD8+_CD103+ | TIL | CD8+_CD103+ | TGCAGTGCTAGTGATGTAGCGGGAGGTTACGAGCAGTACTTC |
| 1289 | 18-TL615-TIL-CD8+_CD103+ | TIL | CD8+_CD103+ | TGTGCCAGCAAGAGCAGGGCCGGCAGTCCCTACGAGCAGTACTTC |
| 1290 | 18-TL615-TIL-CD8+_CD103+ | TIL | CD8+_CD103+ | TGTGCCAGCAGTTACGGCGGAAGGTTCGATGAGCAGTTCTTC |
| 1291 | 18-TL615-TIL-CD8+_CD103+ | TIL | CD8+_CD103+ | TGTGCCAGCAGCTACCCGGGACTAGCGGGAGCATACGAGCAGTACTTC |
| 1292 | 18-TL615-TIL-CD8+_CD103+ | TIL | CD8+_CD103+ | TGTGCCAGCAGCTTAGCAAGCGACCGCTCCTACGAGCAGTACTTC |
| 1293 | 18-TL615-TIL-CD8+_CD103+ | TIL | CD8+_CD103+ | TGTGCCAGCAGCCCCCGGACTGAAGCTTTCTTT |
| 1294 | 18-TL615-TIL-CD8+_CD103+ | TIL | CD8+_CD103+ | TGTGCCAGCAGTTTATATGCCGCAAGTTCAGTAGCTAGCGGGGG |
| | | | | GACAGATACGCAGTGTTTT |
| 1295 | 18-TL615-TIL-CD8+_CD103+ | TIL | CD8+_CD103+ | TGTGCCAGCAGCCAGAGCTTACCCTCGACAGGGGGTCACAATGA |
| | | | | GCAGTTCTTC |
| 1296 | 18-TL615-TIL-CD8+_CD103+ | TIL | CD8+_CD103+ | TGTGCCAGCAGTTACGCCCCCGAGCGGGCTGAAGCTTTCTTT |
| 1297 | 18-TL615-TIL-CD8+_CD103+ | TIL | CD8+_CD103+ | TGTGCCAGCAGCTTCAGAACTAGCAACGCGCAGTATTTT |
| 1298 | 18-TL615-TIL-CD8+_CD103+ | TIL | CD8+_CD103+ | TGTGCTAGCAGCCCCAGTGGAGTAGCGGGAGACGTGGAGACCCAGTACTTC |
| 1299 | 18-TL615-TIL-CD8+_CD103+ | TIL | CD8+_CD103+ | TGTGCCAGTAGTCACCGGGACGGTAATGAAAAACTGTTTTTT |
| 1300 | 18-TL615-TIL-CD8+_CD103+ | TIL | CD8+_CD103+ | TGTGCCAGTAGTATGGATCGTGCTAGCACAGATACGCAGTATTTT |
| 1301 | 18-TL615-TIL-CD8+_CD103+ | TIL | CD8+_CD103+ | TGTGCCAGCCATAATTTCACAGGGGATGAGACCCAGTACTTC |
| 1302 | 18-TL615-TIL-CD8+_CD103+ | TIL | CD8+_CD103+ | TGTGCCAGCAGTTTACGACTAGCGGCTAACACCGGGGAGCTGTTTTTT |
| 1303 | 18-TL615-TIL-CD8+_CD103+ | TIL | CD8+_CD103+ | TGCAGTGCTAGAAAGGGGCTTACCTACGAGCAGTACTTC |
| 1304 | 18-TL615-TIL-CD8+_CD103+ | TIL | CD8+_CD103+ | TGTGCCAGCACCGTGGCTGATTCCTACGAGCAGTACTTC |
| 1305 | 18-TL615-TIL-CD8+_CD103+ | TIL | CD8+_CD103+ | TGTGCCAGCAGTGGGGGCTTCGACAGGGGAGACCACGAGCAGTACTTC |
| 1306 | 18-TL615-TIL-CD8+_CD103+ | TIL | CD8+_CD103+ | TGTGCCAGCAGCGAACAGGGAGGTTCTAGGGGCTACACCTTC |
| 1307 | 18-TL615-TIL-CD8+_CD103+ | TIL | CD8+_CD103+ | TGTGCCAGCAGCCCCTCCGGGACAGGGGGCGAGGAGACCCAGTACTTC |
| 1308 | 18-TL615-TIL-CD8+_CD103+ | TIL | CD8+_CD103+ | TGTGCCAGCAGTTTACGACAGGGAGAGGTCACAGATACGCAGTATTTT |
| 1309 | 18-TL615-TIL-CD8+_CD103+ | TIL | CD8+_CD103+ | TGCAGCGTTGAGGAAGGGGAGCTTTTCTTT |
| 1310 | 18-TL615-TIL-CD8+_CD103+ | TIL | CD8+_CD103+ | TGTGCCATCAGCCCAGGGGACACAGCCTACGAGCAGTACTTC |
| 1311 | 18-TL615-TIL-CD8+_CD103+ | TIL | CD8+_CD103+ | TGTGCCAGCAGTCACAGTCCGAACACTGAAGCTTTCTTT |
| 1312 | 18-TL615-TIL-CD8+_CD103+ | TIL | CD8+_CD103+ | TGTGCCAGCAGCACCATACAGGAGCCGAACACTGAAGCTTTCTTT |
| 1313 | 18-TL615-TIL-CD8+_CD103+ | TIL | CD8+_CD103+ | TGCAGTGCTACCAGCGTAGGAGGGTCCTACAATGAGCAGTTCTTC |
| 1314 | 18-TL615-TIL-CD8+_CD103+ | TIL | CD8+_CD103+ | TGTGCCAGCGAGGACAGGGGATATACGAACACTGAAGCTTTCTTT |
| 1315 | 18-TL615-TIL-CD8+_CD103+ | TIL | CD8+_CD103+ | TGCAGCGTTGAAGAGTTACCAGGAGGGAACACTGAAGCTTTCTTT |
| 1316 | 18-TL615-TIL-CD8+_CD103+ | TIL | CD8+_CD103+ | TGCAGTGCTAGTCGCCGTGAGTGGGAGACCCAGTACTTC |
| 1317 | 18-TL615-TIL-CD8+_CD103+ | TIL | CD8+_CD103+ | TGCAGTGCTGATACTAGCGGGAGGAGGGCCGGGGAGCTGTTTTTT |
| 1318 | 18-TL615-TIL-CD8+_CD103+ | TIL | CD8+_CD103+ | TGTGCCAGCAGCCTAGCGACGCCGCCCTCCTACGAGCAGTACTTC |
| 1319 | 18-TL615-TIL-CD8+_CD103+ | TIL | CD8+_CD103+ | TGTGCCAGCTCACGGACAGGGTACTACAATGAGCAGTTCTTC |
| 1320 | 18-TL615-TIL-CD8+_CD103+ | TIL | CD8+_CD103+ | TGTGCCAGCAGCTTAATCGGGGTTGGGAACACTGAAGCTTTCTTT |
| 1321 | 18-TL615-TIL-CD8+_CD103+ | TIL | CD8+_CD103+ | TGTGCCACCAGTGATTGGGACGGGACTAGCGCCGCCTACGAGCAGTACTTC |
| 1322 | 18-TL615-TIL-CD8+_CD103+ | TIL | CD8+_CD103+ | TGCAGTGCTAGAGATCCTACGCGGGGTGGGAGCTCCTACGAGCAGTACTTC |
| 1323 | 18-TL615-TIL-CD8+_CD103+ | TIL | CD8+_CD103+ | TGTGCCAGCAGCAAAGGGGAGTTGGGGACACCCCCCCGGGAGACCCAGTAC |
| | | | | TTC |
| 1324 | 18-TL615-TIL-CD8+_CD103+ | TIL | CD8+_CD103+ | TGTGCCAGCAGTTGGGGGGCGGGAGGATTGTACGAGCAGTACTTC |
| 1325 | 18-TL615-TIL-CD8+_CD103+ | TIL | CD8+_CD103+ | TGCGCCAGCAGGGTTGTAGCGGGAGGCAATGAGCAGTTCTTC |
| 1326 | 18-TL615-TIL-CD8+_CD103+ | TIL | CD8+_CD103+ | TGTGCCAGCAGCTTAGTTGGTGAAGCTTTCTTT |
| 1327 | 18-TL615-TIL-CD8+_CD103+ | TIL | CD8+_CD103+ | TGTGCCAGCAGTTTATCCCCGGGACTAGCGGACACAGATACGCAGTATTTT |
| 1328 | 18-TL615-TIL-CD8+_CD103+ | TIL | CD8+_CD103+ | TGCAGTGCACCCCTTAGCGGGGGGTTGTACAATGAGCAGTTCTTC |
| 1329 | 18-TL615-TIL-CD8+_CD103+ | TIL | CD8+_CD103+ | TGTGCCAGCAGTTTTGCAGGACAGGGCCGAATGGGAGATACGCAGTATTTT |
| 1330 | 18-TL615-TIL-CD8+_CD103+ | TIL | CD8+_CD103+ | TGTGCCAGCAGCACCAACCGGGTCCTAGGGGACTATGGCTACACCTTC |
| 1331 | 18-TL615-TIL-CD8+_CD103+ | TIL | CD8+_CD103+ | TGCGCCAGCAGCCAAGGGAGGGGCGCACCCACTGAAGCTTTCTTT |
| 1332 | 18-TL615-TIL-CD8+_CD103+ | TIL | CD8+_CD103+ | TGTGCCAGCAGCTTGTGGGGTAATCAGCCCCAGCATTTT |
| 1333 | 18-TL615-TIL-CD8+_CD103+ | TIL | CD8+_CD103+ | TGCGCCAGCAGCCAAGATGGCGGGGGCAGGGAGACCCAGTACTTC |
| 1334 | 18-TL615-TIL-CD8+_CD103+ | TIL | CD8+_CD103+ | TGTGCCAGTAGTATAGGACACGACGGCATGAACACTGAAGCTTTCTTT |
| 1335 | 18-TL615-TIL-CD8+_CD103+ | TIL | CD8+_CD103+ | TGTGCCAGCAGCTTGGAGGGACTAGCGGGGGGCCCTGCCCACAATGAG |
| | | | | CAGTTCTTC |
| 1336 | 19-TL615-TIL-CD8+_CD103− | TIL | CD8+_CD103− | TGTGCCAGCAGCTTGATCAATGAGCAGTTCTTC |
| 1337 | 19-TL615-TIL-CD8+_CD103− | TIL | CD8+_CD103− | TGTGCCAGCAGCTCCTTTTCGGGGTTGGGCTACGAGCAGTACTTC |
| 1338 | 19-TL615-TIL-CD8+_CD103− | TIL | CD8+_CD103− | TGCAGCGTTAAGCGGGAGGGCACCGGGGAGCTGTTTTTT |
| 1339 | 19-TL615-TIL-CD8+_CD103− | TIL | CD8+_CD103− | TGTGCCAGCAGCTTATTTTCGAGGGGAGACATTCAGTACTTC |
| 1340 | 19-TL615-TIL-CD8+_CD103− | TIL | CD8+_CD103− | TGTGCCACCATTCGGACCGGGGGTGCTGGCAATCAGCCCCAGCATTTT |
| 1341 | 19-TL615-TIL-CD8+_CD103− | TIL | CD8+_CD103− | TGTGCCAGCAGCCCGACGGACAGGGGCGCCAGCACTGAAGCTTTCTTT |
| 1342 | 19-TL615-TIL-CD8+_CD103− | TIL | CD8+_CD103− | TGTGCCAGCAGCTGGGGGCTGTCCGGGGCCAGGAACGAGCAGTACTTC |
| 1343 | 19-TL615-TIL-CD8+_CD103− | TIL | CD8+_CD103− | TGCGCCAGCAGCCAAGTCCAAGGGGGTTCCTACGAGCAGTACTTC |
| 1344 | 19-TL615-TIL-CD8+_CD103− | TIL | CD8+_CD103− | TGTGCCAGCATCCCCCACGGGGCTAGCGGGCTATACGAGCAGTACTTC |
| 1345 | 19-TL615-TIL-CD8+_CD103− | TIL | CD8+_CD103− | TGTGCCAGCAGCCCGTTGGGAGGACTGAACACTGAAGCTTTCTTT |
| 1346 | 19-TL615-TIL-CD8+_CD103− | TIL | CD8+_CD103− | TGTGCCAGCAGCTTAGAGGTAGCGGGAGCGAGATACGAGCAGTACTTC |
| 1347 | 19-TL615-TIL-CD8+_CD103− | TIL | CD8+_CD103− | TGCAGCGCCACATTCCCACAGGGGGCCCCTTACAATGAGCAGTTCTTC |
| 1348 | 19-TL615-TIL-CD8+_CD103− | TIL | CD8+_CD103− | TGCAGCGTTGTAACCGGGAGAAATAGCAATCAGCCCCAGCATTTT |
| 1349 | 19-TL615-TIL-CD8+_CD103− | TIL | CD8+_CD103− | TGTGCCAGCAGCTTGGACGGGTCCTACAATGAGCAGTTCTTC |
| 1350 | 19-TL615-TIL-CD8+_CD103− | TIL | CD8+_CD103− | TGTGCCAGCCGATTTCTTAGCGGGAGTCTTGATGAGCAGTTCTTC |
| 1351 | 19-TL615-TIL-CD8+_CD103− | TIL | CD8+_CD103− | TGCGCCAGCAGCCAAGAGTGGGGTTTGGGCCAAAACGGGGAGCTGTTTTTT |
| 1352 | 19-TL615-TIL-CD8+_CD103− | TIL | CD8+_CD103− | TGTGCCAGTAGTATAGGCTGGGGTAGCTCCTACAATGAGCAGTTCTTC |
| 1353 | 19-TL615-TIL-CD8+_CD103− | TIL | CD8+_CD103− | TGTGCCAGCAGGGAGGGGATGGCTGAAGCTTTCTTT |
| 1354 | 19-TL615-TIL-CD8+_CD103− | TIL | CD8+_CD103− | TGTGCCAGCAGCGTAGGAGGGGGTCCCCAAGAGACCCAGTACTTC |
| 1355 | 19-TL615-TIL-CD8+_CD103− | TIL | CD8+_CD103− | TGTGCCAGCAGCCCCGACAAAGACCAGCCCCAGCATTTT |
| 1356 | 19-TL615-TIL-CD8+_CD103− | TIL | CD8+_CD103− | TGTGCCAGCAGCGTAACGGGGACAGGGACCAAAAACATTCAGTACTTC |
| 1357 | 19-TL615-TIL-CD8+_CD103− | TIL | CD8+_CD103− | TGTGCCAGCAGTTTATTATCCGGGAAGCCCCAGCATTTT |
| 1358 | 19-TL615-TIL-CD8+_CD103− | TIL | CD8+_CD103− | TGTGCCAGCACCCTGCACCGGGACAGGGTAGTCGAGCAGTACTTC |
| 1359 | 19-TL615-TIL-CD8+_CD103− | TIL | CD8+_CD103− | TGTGCCAGCAGTTTGTACAATGAGCAGTTCTTC |
| 1360 | 19-TL615-TIL-CD8+_CD103− | TIL | CD8+_CD103− | TGTGCCAGCAGACCTAAAACTAGCGGGAGCCTCGATGAGCAGTTCTTC |
| 1361 | 19-TL615-TIL-CD8+_CD103− | TIL | CD8+_CD103− | TGTGCCAGGAACGAGCAGTACTTC |
| 1362 | 19-TL615-TIL-CD8+_CD103− | TIL | CD8+_CD103− | TGTGCCAGCAGCTACGGGACAGGGACTTACGAGCAGTACTTC |
| 1363 | 19-TL615-TIL-CD8+_CD103− | TIL | CD8+_CD103− | TGTGCCAGCAGCCTATACGGGGGCTATGGCTACACCTTC |
| 1364 | 19-TL615-TIL-CD8+_CD103− | TIL | CD8+_CD103− | TGTGCCAGCAGCTTATCAGGGAGCTGGGGGGACACTGAAGCTTTCTTT |
| 1365 | 19-TL615-TIL-CD8+_CD103− | TIL | CD8+_CD103− | TGTGCCAGCAGTGACTCACTAGCGGATAGCACAGATACGCAGTATTTT |
| 1366 | 19-TL615-TIL-CD8+_CD103− | TIL | CD8+_CD103− | TGTGCCAGTATTCCGGGACTGGCCTACAATGAGCAGTTCTTC |
| 1367 | 19-TL615-TIL-CD8+_CD103− | TIL | CD8+_CD103− | TGTGCCAGCAGCTTGGAAACTCGGGACGAGCAGTACTTC |
| 1368 | 19-TL615-TIL-CD8+_CD103− | TIL | CD8+_CD103− | TGTGCCAGCAGCGAACAGGGGGCTGAAGCTTTCTTT |
| 1369 | 19-TL615-TIL-CD8+_CD103− | TIL | CD8+_CD103− | TGTGCCAGCAGTGTAAGGCAGCTGGGGGAGCAGTACTTC |
| 1370 | 19-TL615-TIL-CD8+_CD103− | TIL | CD8+_CD103− | TGTGCCAGCAGAATCGGACAGAGCTCCTACGAGCAGTACTTC |
| 1371 | 19-TL615-TIL-CD8+_CD103− | TIL | CD8+_CD103− | TGTGCCAGCAGAGAGAGACCAATGGCTGAGACCCAGTACTTC |
| 1372 | 19-TL615-TIL-CD8+_CD103− | TIL | CD8+_CD103− | TGTGCCAGCAGTACCAGCTCCTACGAGCAGTACTTC |
| 1373 | 19-TL615-TIL-CD8+_CD103− | TIL | CD8+_CD103− | TGTGCCAGCAGTTATTCGGGGTCTAGCCGCTACGAGCAGTACTTC |
| 1374 | 19-TL615-TIL-CD8+_CD103− | TIL | CD8+_CD103− | TGTGCCAGCAGCCTTGGTAGTTGGAGCACCGGGGAGCTGTTTTTT |
| 1375 | 19-TL615-TIL-CD8+_CD103− | TIL | CD8+_CD103− | TGTGCCAGCAGTTTTTTTTCCTCAGGACAGGGGGCATACGAGCAGTACTTC |
| 1376 | 19-TL615-TIL-CD8+_CD103− | TIL | CD8+_CD103− | TGCGCCAGCAGCTTGGGACTAGCGGACTACGAGCAGTACTTC |
| 1377 | 19-TL615-TIL-CD8+_CD103− | TIL | CD8+_CD103− | TGTGCCAGCAGCCTCAGGGACACATCCTACGAGCAGTACTTC |
| 1378 | 19-TL615-TIL-CD8+_CD103− | TIL | CD8+_CD103− | TGCGCCAGCAGTGAAACGGACGGTAACACTGAAGCTTTCTTT |
| 1379 | 19-TL615-TIL-CD8+_CD103− | TIL | CD8+_CD103− | TGCGCCAGCAGCTTGGGTAACAATGAGCAGTTCTTC |
| 1380 | 19-TL615-TIL-CD8+_CD103− | TIL | CD8+_CD103− | TGTGCCAGCAGTCCCCACCGCTACGAGCAGTACTTC |
| 1381 | 19-TL615-TIL-CD8+_CD103− | TIL | CD8+_CD103− | TGTGCCAGCAGCTCCTTTTCGGGGTTGGGCTACGAGCAGTACTTC |
| 1382 | 19-TL615-TIL-CD8+_CD103− | TIL | CD8+_CD103− | TGTGCCAGCAGCGTAGAGGACCACCTCACCGGGGAGCTGTTTTTT |
| 1383 | 19-TL615-TIL-CD8+_CD103− | TIL | CD8+_CD103− | TGTGCCAGCAGTCCTAACAGGGGGCCGAGGGAGACTGAAGCTTTCTTT |
| 1384 | 19-TL615-TIL-CD8+_CD103− | TIL | CD8+_CD103− | TGTGCCAGCAGTGACAGGGTACGAAGCGGGGAGCTGTTTTTT |
| 1385 | 19-TL615-TIL-CD8+_CD103− | TIL | CD8+_CD103− | TGCGCCAGCAGCCAAGACGGACTAGCGGGAGACACCGGGGAGCTGTTTTTT |
| 1386 | 19-TL615-TIL-CD8+_CD103− | TIL | CD8+_CD103− | TGTGCCAGTAGCGACGGAATCTCTGGGGCCAACGTCCTGACTTTC |
| 1387 | 19-TL615-TIL-CD8+_CD103− | TIL | CD8+_CD103− | TGCGCCAGCAGCGCCCCGGAGAAACTAGCGGGAGTCTCCTACGAG |
| | | | | CAGTACTTC |
| 1388 | 19-TL615-TIL-CD8+_CD103− | TIL | CD8+_CD103− | TGTGCCAGCTCACCTTTCCCGGGACAGAGTAATGAAAAACTGTTTTTT |
| 1389 | 19-TL615-TIL-CD8+_CD103− | TIL | CD8+_CD103− | TGTGCCACCAGCCAAAGGGGGCGTCTGATCCAGCCCCAGCATTTT |
| 1390 | 19-TL615-TIL-CD8+_CD103− | TIL | CD8+_CD103− | TGCGCCAGCAGCTTGGGAGGGGGAAGCTATGGCTACACCTTC |
| 1391 | 19-TL615-TIL-CD8+_CD103− | TIL | CD8+_CD103− | TGTGCCAGCAGCGAACAGGGAGGTTCTAGGGGCTACACCTTC |
| 1392 | 19-TL615-TIL-CD8+_CD103− | TIL | CD8+_CD103− | TGTGCCAGCAGCGGTTCACAGGGGCTCACAGATACGCAGTATTTT |
| 1393 | 19-TL615-TIL-CD8+_CD103− | TIL | CD8+_CD103− | TGTGCCAGCAGCCCCACCGGGACTAGCGGAGCCAGTGAGCAGTTCTTC |
| 1394 | 19-TL615-TIL-CD8+_CD103− | TIL | CD8+_CD103− | TGTGCCAGCAGCTTAGAGGAGAGTTACAATGAGCAGTTCTTC |
| 1395 | 19-TL615-TIL-CD8+_CD103− | TIL | CD8+_CD103− | TGTGCCAGCAGCTCCACTCAAAATGAGCAGTTCTTC |
| 1396 | 19-TL615-TIL-CD8+_CD103− | TIL | CD8+_CD103− | TGTGCCAGCAGTTCAGGTTACAATGAGCAGTTCTTC |
| 1397 | 19-TL615-TIL-CD8+_CD103− | TIL | CD8+_CD103− | TGTGCCAGCAGTTCTACAGGGGTCTCCTACGAGCAGTACTTC |
| 1398 | 19-TL615-TIL-CD8+_CD103− | TIL | CD8+_CD103− | TGTGCCAGCACGGAGCGGGAGGGTTCCGAGCAGTACTTC |
| 1399 | 19-TL615-TIL-CD8+_CD103− | TIL | CD8+_CD103− | TGCGCCAGCAGCCAAGATTACCTAGCGGGGGGCCGGGCTGAGCAGTTCTTC |
| 1400 | 19-TL615-TIL-CD8+_CD103− | TIL | CD8+_CD103− | TGTGCCAGCAGCATCGGACAGGGCCCTTCCTACGAGCAGTACTTC |
| 1401 | 19-TL615-TIL-CD8+_CD103− | TIL | CD8+_CD103− | TGTGCCAGCAGTTACCAGGGGGGGAACTATGGCTACACCTTC |
| 1402 | 19-TL615-TIL-CD8+_CD103− | TIL | CD8+_CD103− | TGTGCCTCCTCCGGGACAGGGGTCGAGCAGTACTTC |
| 1403 | 19-TL615-TIL-CD8+_CD103− | TIL | CD8+_CD103− | TGCGCCAGCAGTGAAAGAGTTGGCGACGAGCAGTACTTC |
| 1404 | 19-TL615-TIL-CD8+_CD103− | TIL | CD8+_CD103− | TGTGCCAGCAGCACCAACCGGGTCCTAGGGGACTATGGCTACACCTTC |
| 1405 | 19-TL615-TIL-CD8+_CD103− | TIL | CD8+_CD103− | TGTGCCAGCAGCGATGCTTTCACAGATACGCAGTATTTT |
| 1406 | 19-TL615-TIL-CD8+_CD103− | TIL | CD8+_CD103− | TGTGCCAGCAGTTCCCGACTAGCGGGAGGCGGTGAGCAGTTCTTC |
| 1407 | 19-TL615-TIL-CD8+_CD103− | TIL | CD8+_CD103− | TGTGCCAGCAGTTTCAGGGCTTACAATGAGCAGTTCTTC |
| 1408 | 19-TL615-TIL-CD8+_CD103− | TIL | CD8+_CD103− | TGTGCCAGCAGCCCGACAGAGAACACCGGGGAGCTGTTTTTT |
| 1409 | 19-TL615-TIL-CD8+_CD103− | TIL | CD8+_CD103− | TGTGCCAGCAGTTTACTCCACCCTTTCTACGAGCAGTACTTC |
| 1410 | 19-TL615-TIL-CD8+_CD103− | TIL | CD8+_CD103− | TGTGCCAGCAGCTTCCCAGGGGACCACTACGAGCAGTACTTC |
| 1411 | 19-TL615-TIL-CD8+_CD103− | TIL | CD8+_CD103− | TGTGCCAGCAGCTTAGATCGATCGGGCCTAGGGGACTTC |
| 1412 | 19-TL615-TIL-CD8+_CD103− | TIL | CD8+_CD103− | TGTGCCAGCAGGAACTACGAGCAGTACTTC |
| 1413 | 19-TL615-TIL-CD8+_CD103− | TIL | CD8+_CD103− | TGTGCCAGCAGTTTTTACCCAGGGAAGGACTACACCTTC |
| 1414 | 19-TL615-TIL-CD8+_CD103− | TIL | CD8+_CD103− | TGCAGCGCCACATTCCCACAGGGGGCACCTTACAATGAGCAGTTCTTC |
| 1415 | 19-TL615-TIL-CD8+_CD103− | TIL | CD8+_CD103− | TGTGCCAGCAGCTTAGGCCTTGGGGGAAACACTGAAGCTTTCTTT |
| 1416 | 19-TL615-TIL-CD8+_CD103− | TIL | CD8+_CD103− | TGTGCCACCAGCGCCGGGACAGGAGACTACGAGCAGTACTTC |
| 1417 | 19-TL615-TIL-CD8+_CD103− | TIL | CD8+_CD103− | TGTGCCAGCAGCTTAGACATCGTGAGCAATCAGCCCCAGCATTTT |
| 1418 | 19-TL615-TIL-CD8+_CD103− | TIL | CD8+_CD103− | TGTGCCAGCAGCTTGGGGGTTTGGGAGACCCAGTACTTC |
| 1419 | 19-TL615-TIL-CD8+_CD103− | TIL | CD8+_CD103− | TGTGCCAGCAGTTTAGTCCCAGGGAACACTGAAGCTTTCTTT |
| 1420 | 19-TL615-TIL-CD8+_CD103− | TIL | CD8+_CD103− | TGTGCCAGCAGCCCGACTGACTGGGGGAACACCGGGGAGCTGTTTTTT |
| 1421 | 19-TL615-TIL-CD8+_CD103− | TIL | CD8+_CD103− | TGTGCCAGCAGCTCAAAGACAGGGTTGAATGAGCAGTTCTTC |
| 1422 | 19-TL615-TIL-CD8+_CD103− | TIL | CD8+_CD103− | TGTGCCAGCAGCTTAATCTCTGAAGCTTTCTTT |
| 1423 | 19-TL615-TIL-CD8+_CD103− | TIL | CD8+_CD103− | TGTGCCAGCAGCCCAGGGACAGGGGACGCCTTTCGCTCCTAC |
| | | | | AATGAGCAGTTCTTC |
| 1424 | 19-TL615-TIL-CD8+_CD103− | TIL | CD8+_CD103− | TGCGCCAGCAGCTTGGGAGCCAACCGCTATGGCTACACCTTC |
| 1425 | 19-TL615-TIL-CD8+_CD103− | TIL | CD8+_CD103− | TGTGCCAGCAGTTTATCCGCAGGGGGAGGTGGCAATCAGCCCCAGCATTTT |
| 1426 | 19-TL615-TIL-CD8+_CD103− | TIL | CD8+_CD103− | TGTGCCAGCAGCCAAGGTGTTGCCCAGTACTTC |
| 1427 | 19-TL615-TIL-CD8+_CD103− | TIL | CD8+_CD103− | TGCAGTGCTAGAGCGGGACTAGCGGAAATATTCTCCTACAATGAGCAGTTC |
| | | | | TTC |
| 1428 | 19-TL615-TIL-CD8+_CD103− | TIL | CD8+_CD103− | TGTGCCAGCAGCTTAGCCCGGGGGGGTCAGCCCCAGCATTTT |
| 1429 | 19-TL615-TIL-CD8+_CD103− | TIL | CD8+_CD103− | TGTGCCAGCAGCCAGACAGAGAACACCGGGGAGCTGTTTTTT |
| 1430 | 19-TL615-TIL-CD8+_CD103− | TIL | CD8+_CD103− | TGTGCCAGCAGCCAAGTGCAGGGGTTCGCCGGGGAGCTGTTTTTT |
| 1431 | 19-TL615-TIL-CD8+_CD103− | TIL | CD8+_CD103− | TGTGCCAGCAGTGACACCCTCGGGGGGGGTGACACCGGGGAGCTGTTTTTT |
| 1432 | 19-TL615-TIL-CD8+_CD103− | TIL | CD8+_CD103− | TGTGCCAGCAGTCCCGTAGGGAACACTGAAGCTTTCTTT |
| 1433 | 19-TL615-TIL-CD8+_CD103− | TIL | CD8+_CD103− | TGTGCCAGCAGTTCAGCTGGGAAGCAGTATTTT |
| 1434 | 19-TL615-TIL-CD8+_CD103− | TIL | CD8+_CD103− | TGTGCCAGCAGCCCGGGACAGGAGTCCTACGAGCAGTACTTC |
| 1435 | 19-TL615-TIL-CD8+_CD103− | TIL | CD8+_CD103− | TGTGCCAGCAGTCTGACAGGGGGCCGTAATCAGCCCCAGCATTTT |
| 1436 | 19-TL615-TIL-CD8+_CD103− | TIL | CD8+_CD103− | TGCAGTGCTAGAGATCCCCGGCAGGGGGACACCGGGGAGCTGTTTTTT |
| 1437 | 19-TL615-TIL-CD8+_CD103− | TIL | CD8+_CD103− | TGCGCCAGCAGCTTCGATGGGGCCAACGTCCTGACTTTC |
| 1438 | 19-TL615-TIL-CD8+_CD103− | TIL | CD8+_CD103− | TGTGCCAGCAGCAACGCGTGGAATGAGCAGTTCTTC |
| 1439 | 19-TL615-TIL-CD8+_CD103− | TIL | CD8+_CD103− | TGTGCCAGCAGTTCAGGGGCCGGGGAGCTGTTTTTT |
| 1440 | 19-TL615-TIL-CD8+_CD103− | TIL | CD8+_CD103− | TGTGCCAGCGCTATCTCTTTCGGGGAGCAGTACTTC |
| 1441 | 19-TL615-TIL-CD8+_CD103− | TIL | CD8+_CD103− | TGTGCCAGCAGCTTGCTCCCCGGGGGCGATGAGCAGTTCTTC |
| 1442 | 19-TL615-TIL-CD8+_CD103− | TIL | CD8+_CD103− | TGTGCCATCAGTGAGACCCCCTCAACAGTACCTTTTTTT |
| 1443 | 19-TL615-TIL-CD8+_CD103− | TIL | CD8+_CD103− | TGTGCCAGCAGCCTGCTAGCGCAGCAGTTCTTC |
| 1444 | 19-TL615-TIL-CD8+_CD103− | TIL | CD8+_CD103− | TGTGCCAGCAGCGTAGGGTCAGGACCCCCAAATGAGCAGTTCTTC |
| 1445 | 19-TL615-TIL-CD8+_CD103− | TIL | CD8+_CD103− | TGTGCCATCAGTGAAGAGAGGGGGGGAGGAGCAGATACGCAGTATTTT |
| 1446 | 19-TL615-TIL-CD8+_CD103− | TIL | CD8+_CD103− | TGTGCCAGCAGCTGGGGACAGATCAATTCACCCCTCCACTTT |
| 1447 | 19-TL615-TIL-CD8+_CD103− | TIL | CD8+_CD103− | TGTGCCAGCAGCCCTTACATCGGGAGCACAGATACGCAGTATTTT |
| 1448 | 19-TL615-TIL-CD8+_CD103− | TIL | CD8+_CD103− | TGTGCCAGCAGCTTAATAGGCTACGAGCAGTACTTC |
| 1449 | 19-TL615-TIL-CD8+_CD103− | TIL | CD8+_CD103− | TGTGCCAGCAGTCTCCCGACAGGGGCTGAAGATACGCAGTATTTT |
| 1450 | 19-TL615-TIL-CD8+_CD103− | TIL | CD8+_CD103− | TGTGCCAGCAGTTACTCGAATCTAGCGGGAGCCGGGGAGCTGTTTTTT |
| 1451 | 19-TL615-TIL-CD8+_CD103− | TIL | CD8+_CD103− | TGTGCCAGCAGCATGGGGGGACAGAACTATGGCTACACCTTC |
| 1452 | 19-TL615-TIL-CD8+_CD103− | TIL | CD8+_CD103− | TGTGCCAGTAGAGGGGGAACAGTAGACAAAAACATTCAGTACTTC |
| 1453 | 19-TL615-TIL-CD8+_CD103− | TIL | CD8+_CD103− | TGTGCCAGTAGTATTGGCGTCTATGGCTACACCTTC |
| 1454 | 19-TL615-TIL-CD8+_CD103− | TIL | CD8+_CD103− | TGTGCCAGCAGCCAAGAAAAGGGTCGGGTCCCGGACGGCCCTAGC |
| | | | | GTCCCTTACAATGAGCAGTTCTTC |
| 1455 | 19-TL615-TIL-CD8+_CD103− | TIL | CD8+_CD103− | TGCGCCAGCAGCTTGGGGGGCACTGAAGCTTTCTTT |
| 1456 | 19-TL615-TIL-CD8+_CD103− | TIL | CD8+_CD103− | TGTGCCAGCAGTTATGGCGGTAGATACAATGAGCAGTTCTTC |
| 1457 | 19-TL615-TIL-CD8+_CD103− | TIL | CD8+_CD103− | TGCGCCAGCAGCCAAGGAAGGGCCTCGGGGACTGAAGCTTTCTTT |
| 1458 | 19-TL615-TIL-CD8+_CD103− | TIL | CD8+_CD103− | TGCAGCGTTGAAAGGGACGGCGGCGACTATGGCTACACCTTC |
| 1459 | 19-TL615-TIL-CD8+_CD103− | TIL | CD8+_CD103− | TGTGCCACCAGCAATCGACCAGGGACAGCCGAAGAGCAGTTCTTC |
| 1460 | 19-TL615-TIL-CD8+_CD103− | TIL | CD8+_CD103− | TGTGCCAGCAGCTCCCATGGCGGAATCATTCGGCGGACCGAGGGG |
| | | | | AATGAGCAGTTCTTC |
| 1461 | 19-TL615-TIL-CD8+_CD103− | TIL | CD8+_CD103− | TGCAGTGCTAGTCGGCGGGAGAGCTACGAGCAGTACTTC |
| 1462 | 19-TL615-TIL-CD8+_CD103− | TIL | CD8+_CD103− | TGTGCCAGCAGTTACTCGACCGCGGCAGATGAAGAGACCCAGTACTTC |
| 1463 | 19-TL615-TIL-CD8+_CD103− | TIL | CD8+_CD103− | TGCGCCAGCAGTCTCGACAGCTCCTACGAGCAGTACTTC |
| 1464 | 19-TL615-TIL-CD8+_CD103− | TIL | CD8+_CD103− | TGCGCCAGCAGCCAAGATCCAGGGGGCCCTAACTCCTACA |
| | | | | ATGAGCAGTTCTTC |
| 1465 | 19-TL615-TIL-CD8+_CD103− | TIL | CD8+_CD103− | TGTGCCAGCAGCCAAGACCCCCTTTTGGTGGGGGTGGACA |
| | | | | CTGAAGCTTTCTTT |
| 1466 | 19-TL615-TIL-CD8+_CD103− | TIL | CD8+_CD103− | TGTGCCAGCAGTTTGGGGGTTAATATGAACACTGAAGCTTTCTTT |
| 1467 | 19-TL615-TIL-CD8+_CD103− | TIL | CD8+_CD103− | TGTGCCAGCAGTCTGACAGGGAGTCGCAATCAGCCCCAGCATTTT |
| 1468 | 19-TL615-TIL-CD8+_CD103− | TIL | CD8+_CD103− | TGCAGTGCTAGTCCAGATACCTACGAGCAGTACTTC |
| 1469 | 19-TL615-TIL-CD8+_CD103− | TIL | CD8+_CD103− | TGTGCCAGCAGCTTAGGAGGGGGAGCCAAAAACATTCAGTACTTC |
| 1470 | 19-TL615-TIL-CD8+_CD103− | TIL | CD8+_CD103− | TGTGCCAGCAGCTACGGACTAGCCTACAATGAGCAGTTCTTC |
| 1471 | 19-TL615-TIL-CD8+_CD103− | TIL | CD8+_CD103− | TGCAGTGCGCCGACAGGGGGCTGGTATGGCTACACCTTC |
| 1472 | 19-TL615-TIL-CD8+_CD103− | TIL | CD8+_CD103− | TGTGCCAGCAAAGGGGTCACCGGGGAGCTGTTTTTT |
| 1473 | 19-TL615-TIL-CD8+_CD103− | TIL | CD8+_CD103− | TGTGCCAGCACCCTGCAGGGGTTAGCGGGAGAAGAGCAGTTCTTC |
| 1474 | 19-TL615-TIL-CD8+_CD103− | TIL | CD8+_CD103− | TGTGCCAGCAGCCCCACCGGTGGGTACTACGAGCAGTACTTC |
| 1475 | 19-TL615-TIL-CD8+_CD103− | TIL | CD8+_CD103− | TGTGCCAGCAGTTTTAGCAAGGGAACTGAAGCTTTCTTT |
| 1476 | 19-TL615-TIL-CD8+_CD103− | TIL | CD8+_CD103− | TGTGCCAGCAGTTTACGATTTACAGGGGAGGTGGAGCTGTTTTTT |
| 1477 | 19-TL615-TIL-CD8+_CD103− | TIL | CD8+_CD103− | TGTGCCAGCAGCCCCCCGGGCACTGAAGCTTTCTTT |
| 1478 | 19-TL615-TIL-CD8+_CD103− | TIL | CD8+_CD103− | TGTGCCAGCTCACCACTACAGGGTTATGCTGAAGCTTTCTTT |
| 1479 | 19-TL615-TIL-CD8+_CD103− | TIL | CD8+_CD103− | TGTGCCAGCAGTTTAAGACTCCCCGGGCACACTGAAGCTTTCTTT |
| 1480 | 19-TL615-TIL-CD8+_CD103− | TIL | CD8+_CD103− | TGTGCCAGCAGCCCTGGGACAGGGGGCGAGGAGACCCAGTACTTC |
| 1481 | 19-TL615-TIL-CD8+_CD103− | TIL | CD8+_CD103− | TGTGCCAGCAGCCCCCAGCGGGGCCTAGGCTATGGCTACACCTTC |
| 1482 | 19-TL615-TIL-CD8+_CD103− | TIL | CD8+_CD103− | TGCGCCAGCAGCCAGGATCCTACACCTACCTACGAGCAGTACTTC |
| 1483 | 19-TL615-TIL-CD8+_CD103− | TIL | CD8+_CD103− | TGTGCCAGCAGCCTGGGACTATCTTACAATGAGCAGTTCTTC |
| 1484 | 19-TL615-TIL-CD8+_CD103− | TIL | CD8+_CD103− | TGTGCCAGCAGCACCAACTCCGGGACCGCGAGGTCACCCCTCCACTTT |
| 1485 | 19-TL615-TIL-CD8+_CD103− | TIL | CD8+_CD103− | TGTGCCAGCAGTCCCGACAGCTACAATCAGCCCCAGCATTTT |
| 1486 | 19-TL615-TIL-CD8+_CD103− | TIL | CD8+_CD103− | TGTGCCAGCAGTTTTCTCCGGGACTATAGCAATCAGCCCCAGCATTTT |
| 1487 | 19-TL615-TIL-CD8+_CD103− | TIL | CD8+_CD103− | TGTGCCAGCAGCCTGGGCCGTGGCGAGCAGTACTTC |
| 1488 | 19-TL615-TIL-CD8+_CD103− | TIL | CD8+_CD103− | TGTGCTAGCAGCTTAGGTCCTAGCGTGAGGGAGACCCAGTACTTC |
| 1489 | 19-TL615-TIL-CD8+_CD103− | TIL | CD8+_CD103− | TGTGCCAGCAGTATGACTAGCCGGACGGATGAGCAGTACTTC |
| 1490 | 19-TL615-TIL-CD8+_CD103− | TIL | CD8+_CD103− | TGTGCCAGCAGTCCACAACCGGGACCTTTCTACGAGCAGTACTTC |
| 1491 | 19-TL615-TIL-CD8+_CD103− | TIL | CD8+_CD103− | TGTGCCAGCAGTAACCCTATAGCGGGAGGACCCTACAATGAGCAGTTCTTC |
| 1492 | 19-TL615-TIL-CD8+_CD103− | TIL | CD8+_CD103− | TGTGCCATCAGTGAGTCGGGGAGCACCTACGAAGAGACCCAGTACTTC |
| 1493 | 19-TL615-TIL-CD8+_CD103− | TIL | CD8+_CD103− | TGTGCCAGCAGCCAAGACTTTCCAGGGTCTAATCAGCCCCAGCATTTT |
| 1494 | 19-TL615-TIL-CD8+_CD103− | TIL | CD8+_CD103− | TGCGCCAGCAGCCCAGCGGGAGCAAATCAAGAGACCCAGTACTTC |
| 1495 | 19-TL615-TIL-CD8+_CD103− | TIL | CD8+_CD103− | TGTGCCAGCAGTTTAAAGGGTTTGAACACTGAAGCTTTCTTT |
| 1496 | 19-TL615-TIL-CD8+_CD103− | TIL | CD8+_CD103− | TGTGCCAGCAGCCCCACTAACTATGGCTACACCTTC |
| 1497 | 19-TL615-TIL-CD8+_CD103− | TIL | CD8+_CD103− | TGCGCCAGCAGGGGTACTAGCGGGGGCGCAGATACGCAGTATTTT |
| 1498 | 19-TL615-TIL-CD8+_CD103− | TIL | CD8+_CD103− | TGTGCCAGCAGTCCAACAGGGGATCCTTACAATGAGCAGTTCTTC |
| 1499 | 19-TL615-TIL-CD8+_CD103− | TIL | CD8+_CD103− | TGTGCCAGCAGCTTAGAAACAGGCTTCAATCAGCCCCAGCATTTT |
| 1500 | 19-TL615-TIL-CD8+_CD103− | TIL | CD8+_CD103− | TGTGCCAGCAGCTTGTGGGTAGCGGGATGGGATGAGCAGTTCTTC |
| 1501 | 19-TL615-TIL-CD8+_CD103− | TIL | CD8+_CD103− | TGTGCCAGCAGAGGACCCTCCTACGAGCAGTACTTC |
| 1502 | 19-TL615-TIL-CD8+_CD103− | TIL | CD8+_CD103− | TGTGCCAGCAGCACTTCGGACAGGGGGCTTGCCGGGGAGCTGTTTTTT |
| 1503 | 19-TL615-TIL-CD8+_CD103− | TIL | CD8+_CD103− | TGTGCCAGCAGCCGCGGGACTAGCGGTTACAATGAGCAGTTCTTC |
| 1504 | 19-TL615-TIL-CD8+_CD103− | TIL | CD8+_CD103− | TGTGCCAGCAGCCCGATGGGACTAGCGGGAGTCGAGTCCAAAAAC |
| | | | | ATTCAGTACTTC |
| 1505 | 19-TL615-TIL-CD8+_CD103− | TIL | CD8+_CD103− | TGTGCCAGCAGCTTAGCCCCCGGGGCCGAGCAGTACTTC |
| 1506 | 19-TL615-TIL-CD8+_CD103− | TIL | CD8+_CD103− | TGTGCCAGCAGTTACTTACCATCGGGGGAGACCCAGTACTTC |
| 1507 | 19-TL615-TIL-CD8+_CD103− | TIL | CD8+_CD103− | TGTGCCAGCAGCTTGGGCCCTGGGGGTAGTTCACCCCTCCACTTT |
| 1508 | 19-TL615-TIL-CD8+_CD103− | TIL | CD8+_CD103− | TGTGCCACCAGCGGTGCGGGGAAGGACTATGGCTACACCTTC |
| 1509 | 19-TL615-TIL-CD8+_CD103− | TIL | CD8+_CD103− | TGTGCCAGCAGGCCTTTACAGGGGTGGAGCAATCAGCCCCAGCATTTT |
| 1510 | 19-TL615-TIL-CD8+_CD103− | TIL | CD8+_CD103− | TGTGCCAGCAGCTGGGGGCTGGCCGGGGCCAGGAACGAGCAGTACTTC |
| 1511 | 19-TL615-TIL-CD8+_CD103− | TIL | CD8+_CD103− | TGTGCCAGCAGCTTAGATATAGTAGCGGGAGGGGGCGAGCAGTACTTC |
| 1512 | 19-TL615-TIL-CD8+_CD103− | TIL | CD8+_CD103− | TGTGCCACCCTAGGGCGGGAGGGGAGCAATGAGCAGTTCTTC |
| 1513 | 19-TL615-TIL-CD8+_CD103− | TIL | CD8+_CD103− | TGTGCCAGCAGCCTCTTACGGGAGGTCACAGATACGCAGTATTTT |
| 1514 | 19-TL615-TIL-CD8+_CD103− | TIL | CD8+_CD103− | TGCGCCAGCACCCCAGGGATCAACACCGGGGAGCTGTTTTTT |
| 1515 | 19-TL615-TIL-CD8+_CD103− | TIL | CD8+_CD103− | TGTGCCACCAGCAGAGATCGGGAAGGCGGCGCTGAAGCTTTCTTT |
| 1516 | 19-TL615-TIL-CD8+_CD103− | TIL | CD8+_CD103− | TGTGCCAGCAGCTTAAGGCAGGGGCCGTCCTACGAGCAGTACTTC |
| 1517 | 19-TL615-TIL-CD8+_CD103− | TIL | CD8+_CD103− | TGTGCCAGCAGCCCCCAACGAACCACGAACACTGAAGCTTTCTTT |
| 1518 | 19-TL615-TIL-CD8+_CD103− | TIL | CD8+_CD103− | TGTGCCAGTAGTATAGGACTGTCTAGTCTCAATGAGCAGTTCTTC |
| 1519 | 19-TL615-TIL-CD8+_CD103− | TIL | CD8+_CD103− | TGCGCCAGCTTACCTGGCATAGCCCCCATCAGCTCCTACGAGCAGTACTTC |
| 1520 | 19-TL615-TIL-CD8+_CD103− | TIL | CD8+_CD103− | TGCGCCAGCAGGGTTGTAGCGGGAGGCAATGAGCAGTTCTTC |
| 1521 | 19-TL615-TIL-CD8+_CD103− | TIL | CD8+_CD103− | TGCGCCAGCAGCTTGACAGAGGACAATGAGCAGTTCTTC |
| 1522 | 19-TL615-TIL-CD8+_CD103− | TIL | CD8+_CD103− | TGCAGCGTTACTCAGGTGGACACTGGAAACACCATATATTTT |
| 1523 | 19-TL615-TIL-CD8+_CD103− | TIL | CD8+_CD103− | TGTGCCAGCAGCCAAGGGGGGAGCAAATACGAGCAGTACTTC |
| 1524 | 19-TL615-TIL-CD8+_CD103− | TIL | CD8+_CD103− | TGTGCCAGCAGCTTGTGGGGTAATCAGCCCCAGCATTTT |
| 1525 | 19-TL615-TIL-CD8+_CD103− | TIL | CD8+_CD103− | TGCAGTGCCACCTACGCAGGGAGGTTTTGGGAGCCCCAGCATTTT |
| 1526 | 19-TL615-TIL-CD8+_CD103− | TIL | CD8+_CD103− | TGCGCCAGCAGCTTGTCAGGAGACCGTGAAGCTTTCTTT |
| 1527 | 19-TL615-TIL-CD8+_CD103− | TIL | CD8+_CD103− | TGCAGTGCTCGCGGGAGATCCGCCTACGAGCAGTACTTC |
| 1528 | 55-TL661-TIL-CD8+_CD103+ | TIL | CD8+_CD103+ | TGTGCCAGCAGCTTTTCGGGAGGGCCCTACAATGAGCAGTTCTTC |
| 1529 | 55-TL661-TIL-CD8+_CD103+ | TIL | CD8+_CD103+ | TGTGCCAGCAGCCCAAGTGGATTTTCAACTAATGAAAAACTGTTTTTT |
| 1530 | 55-TL661-TIL-CD8+_CD103+ | TIL | CD8+_CD103+ | TGCAGTGCTAGAGATAGGAGCGGGAGAGAGTATTTT |
| 1531 | 55-TL661-TIL-CD8+_CD103+ | TIL | CD8+_CD103+ | TGTGCCAGCAGTGATACCGGCAGCACCTCTTATGGCTACACCTTC |
| 1532 | 55-TL661-TIL-CD8+_CD103+ | TIL | CD8+_CD103+ | TGTGCCAGCAGTTCAGCGGGATTGGGCCACGAGCAGTACTTC |
| 1533 | 55-TL661-TIL-CD8+_CD103+ | TIL | CD8+_CD103+ | TGCGCCAGCAGCTTGACTAGCGGTATTTACAATGAGCAGTTCTTC |
| 1534 | 55-TL661-TIL-CD8+_CD103+ | TIL | CD8+_CD103+ | TGTGCCAGCAGCCTCGGACAGGACACCGGGGAGCTGTTTTTT |
| 1535 | 55-TL661-TIL-CD8+_CD103+ | TIL | CD8+_CD103+ | TGTGCCAGCAGCCCAGGGAAGGGAACTGAAGCTTTCTTT |
| 1536 | 55-TL661-TIL-CD8+_CD103+ | TIL | CD8+_CD103+ | TGTGCCAGCAGCTATGACTGGAGACTCAAAGAGACCCAGTACTTC |
| 1537 | 55-TL661-TIL-CD8+_CD103+ | TIL | CD8+_CD103+ | TGTGCCAGCAGCTCTCCGGGACAGTCCTACGAGCAGTACTTC |
| 1538 | 55-TL661-TIL-CD8+_CD103+ | TIL | CD8+_CD103+ | TGTGCCAGCAGCCAAGGTGGAGGGGGCACTGAAGCTTTCTTT |
| 1539 | 55-TL661-TIL-CD8+_CD103+ | TIL | CD8+_CD103+ | TGTGCCAGCAGTCCTACAGGCCAAGAGACCCAGTACTTC |
| 1540 | 55-TL661-TIL-CD8+_CD103+ | TIL | CD8+_CD103+ | TGTGCCAGCAGCGCCTCGTTGACAGGGCCATTGTCCGAGCAGTACTTC |
| 1541 | 55-TL661-TIL-CD8+_CD103+ | TIL | CD8+_CD103+ | TGCGCCAGCAGCCCTCTTATTGGAGCGGTAAGCTCCTACGAGCAGTACTTC |
| 1542 | 55-TL661-TIL-CD8+_CD103+ | TIL | CD8+_CD103+ | TGCGCCAGCAGCCAATTCGGGGGAGACACTGAAGCTTTCTTT |
| 1543 | 55-TL661-TIL-CD8+_CD103+ | TIL | CD8+_CD103+ | TGTGCCAGCAGCACCAGCCCAGGGGGCCGGGGCTACACCTTC |
| 1544 | 55-TL661-TIL-CD8+_CD103+ | TIL | CD8+_CD103+ | TGTGCCAGCCGTCCGGGGGCAGGAAACACTGAAGCTTTCTTT |
| 1545 | 55-TL661-TIL-CD8+_CD103+ | TIL | CD8+_CD103+ | TGTGCCAGTAGTATGTTTTCTCCGGTCTCCTACAATGAGCAGTTCTTC |
| 1546 | 55-TL661-TIL-CD8+_CD103+ | TIL | CD8+_CD103+ | TGTGCCAGCAGCCATGGGGATCCCAATGAGCAGTTCTTC |
| 1547 | 55-TL661-TIL-CD8+_CD103+ | TIL | CD8+_CD103+ | TGTGCCAGCAGCGGGGGACAGGGGGAATGGGAGCTGTTTTTT |
| 1548 | 55-TL661-TIL-CD8+_CD103+ | TIL | CD8+_CD103+ | TGCAGCGTCCTACTAGCGAACACAGATACGCAGTATTTT |
| 1549 | 55-TL661-TIL-CD8+_CD103+ | TIL | CD8+_CD103+ | TGTGCCAGCAGCTGGGGCAGGGGCGCAAATTCACCCCTCCACTTT |
| 1550 | 55-TL661-TIL-CD8+_CD103+ | TIL | CD8+_CD103+ | TGCGCCAGCAGCCAAGGGACATTTCCCGAACCGGGCTACACCTTC |
| 1551 | 55-TL661-TIL-CD8+_CD103+ | TIL | CD8+_CD103+ | TGTGCCAGTAGTATGGCGGCGGGGGGGCGCATTGAGCAGTTCTTC |
| 1552 | 55-TL661-TIL-CD8+_CD103+ | TIL | CD8+_CD103+ | TGTGCCAGCAGGGGACAGTCTGTGGACACCGGGGAGCTGTTTTTT |
| 1553 | 55-TL661-TIL-CD8+_CD103+ | TIL | CD8+_CD103+ | TGTGCCAGCAGCGACAGGGATCTCTCTGGAAACACCATATATTTT |
| 1554 | 55-TL661-TIL-CD8+_CD103+ | TIL | CD8+_CD103+ | TGCAGCGTTGAAGCCGGGACAGGTGACGAGCAGTACTTC |
| 1555 | 55-TL661-TIL-CD8+_CD103+ | TIL | CD8+_CD103+ | TGCAGTGCTAGAGATGACCTTAAACCTGCCGAGCAGTACTTC |
| 1556 | 55-TL661-TIL-CD8+_CD103+ | TIL | CD8+_CD103+ | TGCGCCAGCAGCTTGGCCGAGAGGGCAGGGGAAGAGACCCAGTACTTC |
| 1557 | 55-TL661-TIL-CD8+_CD103+ | TIL | CD8+_CD103+ | TGCGCCAGCAGCTTCGGCACGGAGGCTTTCTTT |
| 1558 | 55-TL661-TIL-CD8+_CD103+ | TIL | CD8+_CD103+ | TGTGCCAGCAGCCAAGACAGCTATGGCTACACCTTC |
| 1559 | 55-TL661-TIL-CD8+_CD103+ | TIL | CD8+_CD103+ | TGCGCCAGCAGCTTGGACTTGGCAGGCCTTGAAGCTTTCTTT |
| 1560 | 55-TL661-TIL-CD8+_CD103+ | TIL | CD8+_CD103+ | TGTGCCATCAGTGCGGGACAGGTAACTGTTCGCTACGAGCAGTACTTC |
| 1561 | 55-TL661-TIL-CD8+_CD103+ | TIL | CD8+_CD103+ | TGTGCCAGCAGCTCCCTCTATGAGCAGTTCTTC |
| 1562 | 55-TL661-TIL-CD8+_CD103+ | TIL | CD8+_CD103+ | TGTGCCAGCAGTCCAGGGTATAGCAATCAGCCCCAGCATTTT |
| 1563 | 55-TL661-TIL-CD8+_CD103+ | TIL | CD8+_CD103+ | TGTGCCAGCAGTGAACTCGTCTCTGGAAACACCATATATTTT |
| 1564 | 55-TL661-TIL-CD8+_CD103+ | TIL | CD8+_CD103+ | TGTGCCAGCAGGCGGGGTGCGAGACTACAAGAGACCCAGTACTTC |
| 1565 | 55-TL661-TIL-CD8+_CD103+ | TIL | CD8+_CD103+ | TGTGCCAGCAGTGCGAAGCCTTTCAATGAGCAGTTCTTC |
| 1566 | 55-TL661-TIL-CD8+_CD103+ | TIL | CD8+_CD103+ | TGTGCCAGCAGCGTCTCGGGACTTCACAATGAGCAGTTCTTC |
| 1567 | 55-TL661-TIL-CD8+_CD103+ | TIL | CD8+_CD103+ | TGTGCCAGCAGTTTAAGTCACAGGGACCCCTATGGCTACACCTTC |
| 1568 | 55-TL661-TIL-CD8+_CD103+ | TIL | CD8+_CD103+ | TGTGCCAGCAGTCCTCCGGGACTAGGTACAGATACGCAGTATTTT |
| 1569 | 55-TL661-TIL-CD8+_CD103+ | TIL | CD8+_CD103+ | TGTGCCAGCAGTTTTCGTGGGCTTGGAGCTTTCTTT |
| 1570 | 55-TL661-TIL-CD8+_CD103+ | TIL | CD8+_CD103+ | TGTGCCAGCAGTGACGGGGGCCTTCACACAGATACGCAGTATTTT |
| 1571 | 55-TL661-TIL-CD8+_CD103+ | TIL | CD8+_CD103+ | TGTGCCAGCAGCTTCAATCCCTACAATGAGCAGTTCTTC |
| 1572 | 55-TL661-TIL-CD8+_CD103+ | TIL | CD8+_CD103+ | TGTGCCAGCAGCCAAGATTCGGGGAGTGGCAATCAGCCCCAGCATTTT |
| 1573 | 55-TL661-TIL-CD8+_CD103+ | TIL | CD8+_CD103+ | TGTGCCAGCGGAGGCAGGGGGAACTACGAGCAGTACTTC |
| 1574 | 55-TL661-TIL-CD8+_CD103+ | TIL | CD8+_CD103+ | TGTGCCAGCAGTTGGGGACAGAACACCGGGGAGCTGTTTTTT |
| 1575 | 55-TL661-TIL-CD8+_CD103+ | TIL | CD8+_CD103+ | TGTGCCAGCACCACAGGGCCGCCTTTGATAGCTTTCTTT |
| 1576 | 55-TL661-TIL-CD8+_CD103+ | TIL | CD8+_CD103+ | TGTGCCAGCAGTTTATCAAATTCGGGCACTGAAGCTTTCTTT |
| 1577 | 55-TL661-TIL-CD8+_CD103+ | TIL | CD8+_CD103+ | TGTGCCAGCAGCCCTTTGGGGGTGAACACTGAAGCTTTCTTT |
| 1578 | 55-TL661-TIL-CD8+_CD103+ | TIL | CD8+_CD103+ | TGTGCCAGCAGCCACCCGGGACAGGGGATTTACGAGCAGTACTTC |
| 1579 | 55-TL661-TIL-CD8+_CD103+ | TIL | CD8+_CD103+ | TGCGCCAGCAGTGAGTCTACAGGGGAGGACCAGCCCCAGCATTTT |
| 1580 | 55-TL661-TIL-CD8+_CD103+ | TIL | CD8+_CD103+ | TGTGCCAGCAGCTTGTCGTCGGGGAAAGAGCAGTACTTC |
| 1581 | 55-TL661-TIL-CD8+_CD103+ | TIL | CD8+_CD103+ | TGTGCCAGCAGCTTTGATCTGGCGGCGGGGTCATCCACA |
| | | | | GATACGCAGTATTTT |
| 1582 | 55-TL661-TIL-CD8+_CD103+ | TIL | CD8+_CD103+ | TGCGCCAGCAGCACTTTGCCCTCAGGGGTTTACGAGCAGTACTTC |
| 1583 | 55-TL661-TIL-CD8+_CD103+ | TIL | CD8+_CD103+ | TGTGCCAGCAGCTTACGGACAGGGGTAAAAGAGACCCAGTACTTC |
| 1584 | 55-TL661-TIL-CD8+_CD103+ | TIL | CD8+_CD103+ | TGCGCCAGCAGTGAACGCGGGGGGCCCCATGAGCAGTTCTTC |
| 1585 | 55-TL661-TIL-CD8+_CD103+ | TIL | CD8+_CD103+ | TGTGCCAGCAGCGCTCCGGGACAGGGGTTCCCCGAGCAGTACTTC |
| 1586 | 55-TL661-TIL-CD8+_CD103+ | TIL | CD8+_CD103+ | TGCAGCGTTGACGACGGGGTTGATGAGCAGTACTTC |
| 1587 | 55-TL661-TIL-CD8+_CD103+ | TIL | CD8+_CD103+ | TGCGCCAGCAGTGGGGGGGCAGATACGCAGTATTTT |
| 1588 | 55-TL661-TIL-CD8+_CD103+ | TIL | CD8+_CD103+ | TGTGCCAGCAGTTTATCGTGGGACAGGGATTGGATA |
| | | | | CTCAACAATGAGCAGTTCTTC |
| 1589 | 55-TL661-TIL-CD8+_CD103+ | TIL | CD8+_CD103+ | TGCCAAGCCGTGGGGGACGTGGCAGATACGCAGTATTTT |
| 1590 | 55-TL661-TIL-CD8+_CD103+ | TIL | CD8+_CD103+ | TGTGCCAGCAGCTATACTTCCGGCTCCTACAATGAGCAGTTCTTC |
| 1591 | 55-TL661-TIL-CD8+_CD103+ | TIL | CD8+_CD103+ | TGCGCCAGCAGCGCCGATAGCGGGAGAGTCGCTGAGCAGTTCTTC |
| 1592 | 55-TL661-TIL-CD8+_CD103+ | TIL | CD8+_CD103+ | TGCGCCAGCAGCTTGGTAGCACCCAATGAGCAGTTCTTC |
| 1593 | 55-TL661-TIL-CD8+_CD103+ | TIL | CD8+_CD103+ | TGTGCCAGTAGTTCCCACACTGAAGCTTTCTTT |
| 1594 | 55-TL661-TIL-CD8+_CD103+ | TIL | CD8+_CD103+ | TGTGCTAGCAGCCTAGCGGGCGGGGACTACCACGAGCAGTACTTC |
| 1595 | 55-TL661-TIL-CD8+_CD103+ | TIL | CD8+_CD103+ | TGTGCCAGCAGTAAGCTAGGGACAGGGAGGGACAATGAGCAGTTCTTC |
| 1596 | 55-TL661-TIL-CD8+_CD103+ | TIL | CD8+_CD103+ | TGTGCCAGCTCACCGGAGGACAGGGTGAGGAGCACTGAAGCTTTCTTT |
| 1597 | 55-TL661-TIL-CD8+_CD103+ | TIL | CD8+_CD103+ | TGTGCCAGCAGTTTATCGGGACTTTCCCGGAGGAGATACGAGCAGTACTTC |
| 1598 | 55-TL661-TIL-CD8+_CD103+ | TIL | CD8+_CD103+ | TGCGCCAGCAGCCAAGTTCTAGCTCCTAGCACAGATACGCAGTATTTT |
| 1599 | 55-TL661-TIL-CD8+_CD103+ | TIL | CD8+_CD103+ | TGTGCCAGCAGTTTATACCCCCCGGGGAGCAATCAGCCCCAGCATTTT |
| 1600 | 55-TL661-TIL-CD8+_CD103+ | TIL | CD8+_CD103+ | TGTGCCAGCAGTTTCCTAGAGAACCAAGAGACCCAGTACTTC |
| 1601 | 55-TL661-TIL-CD8+_CD103+ | TIL | CD8+_CD103+ | TGTGCCAGCAGCTTAGGCGGGGGCCTCGATGGCTACACCTTC |
| 1602 | 55-TL661-TIL-CD8+_CD103+ | TIL | CD8+_CD103+ | TGTGCCAGCAGCTTGGGTTACAATGAGCAGTTCTTC |
| 1603 | 55-TL661-TIL-CD8+_CD103+ | TIL | CD8+_CD103+ | TGCAGTGCTAGAGATCCCAGGGGGTATGGCTACTATGGCTACACCTTC |
| 1604 | 55-TL661-TIL-CD8+_CD103+ | TIL | CD8+_CD103+ | TGTGCCAGCAGTTTAGCGTTACTGACAGATACGCAGTATTTT |
| 1605 | 55-TL661-TIL-CD8+_CD103+ | TIL | CD8+_CD103+ | TGCGCCAGCAGCCAAGAGTACGGGGGGGCCCAGAATGAGCAGTTCTTC |
| 1606 | 55-TL661-TIL-CD8+_CD103+ | TIL | CD8+_CD103+ | TGTGCCTGGAGCGTAGCACGGTACACTGAAGCTTTCTTT |
| 1607 | 55-TL661-TIL-CD8+_CD103+ | TIL | CD8+_CD103+ | TGCGCCACACTGGGGTCCACCGGGGAGCTGTTTTTT |
| 1608 | 55-TL661-TIL-CD8+_CD103+ | TIL | CD8+_CD103+ | TGTGCCAGCAGCGTGACAGGGGGGACTGAAGCTTTCTTT |
| 1609 | 55-TL661-TIL-CD8+_CD103+ | TIL | CD8+_CD103+ | TGTGCCAGCAGTGAAGTTCTAACCTTCTATGGCAATCAGCCCCAGCATTTT |
| 1610 | 55-TL661-TIL-CD8+_CD103+ | TIL | CD8+_CD103+ | TGCGCCAGCAGCCAAGATGTTTGGACAGCCTATGGCTACACCTTC |
| 1611 | 55-TL661-TIL-CD8+_CD103+ | TIL | CD8+_CD103+ | TGCGCCAGCAGCTTGTTTTCCTCGGGGATCTACGAGCAGTACTTC |
| 1612 | 55-TL661-TIL-CD8+_CD103+ | TIL | CD8+_CD103+ | TGTGCCAGCAGCTTAGCAGGAATAGACAACTATGGCTACACCTTC |
| 1613 | 55-TL661-TIL-CD8+_CD103+ | TIL | CD8+_CD103+ | TGCGCCAGCAGCCAAGTAGCGGGGCCATACAATGAGCAGTTCTTC |
| 1614 | 55-TL661-TIL-CD8+_CD103+ | TIL | CD8+_CD103+ | TGCAGCGTTGTGGTGCAGTACAATGAGCAGTTCTTC |
| 1615 | 55-TL661-TIL-CD8+_CD103+ | TIL | CD8+_CD103+ | TGCGCCAGCAGCTTGTCTCTAGCGGGAACCCTCCAAGAGACCCAGTACTTC |
| 1616 | 55-TL661-TIL-CD8+_CD103+ | TIL | CD8+_CD103+ | TGTGCCAGCAGCTTGGGGAGGGGATTAGGGTACGAGCAGTACTTC |
| 1617 | 55-TL661-TIL-CD8+_CD103+ | TIL | CD8+_CD103+ | TGCAGCGTTGGTTACGGCTACGAGCAGTACTTC |
| 1618 | 55-TL661-TIL-CD8+_CD103+ | TIL | CD8+_CD103+ | TGTGCCAGTTCCCTTGGGACTAGCGGGGCCCCATCCTACGAGCAGTACTTC |
| 1619 | 55-TL661-TIL-CD8+_CD103+ | TIL | CD8+_CD103+ | TGCGCCAGCAGCGCAGGACCTGAGCAGTTCTTC |
| 1620 | 55-TL661-TIL-CD8+_CD103+ | TIL | CD8+_CD103+ | TGCGCCAGCAGCTGGGCCCCGGGACAGCCCGACAATGAGCAGTTCTTC |
| 1621 | 55-TL661-TIL-CD8+_CD103+ | TIL | CD8+_CD103+ | TGCGCCAGGAGACAGGGCTATGGCTACACCTTC |
| 1622 | 55-TL661-TIL-CD8+_CD103+ | TIL | CD8+_CD103+ | TGTGCCAGCAGGCACGGCACTAGCGGTTACAATGAGCAGTTCTTC |
| 1623 | 55-TL661-TIL-CD8+_CD103+ | TIL | CD8+_CD103+ | TGCAGTGCTAGAGATTCTACAGACTCTGGGGCCAACGTCCTGACTTTC |
| 1624 | 55-TL661-TIL-CD8+_CD103+ | TIL | CD8+_CD103+ | TGTGCCAGCAGGATCCCGGGGTTGTACGAGCAGTACTTC |
| 1625 | 55-TL661-TIL-CD8+_CD103+ | TIL | CD8+_CD103+ | TGCAGCGTTGACGAGACAGGGGACACTGAAGCTTTCTTT |
| 1626 | 55-TL661-TIL-CD8+_CD103+ | TIL | CD8+_CD103+ | TGTGCCAGCAGTTTAGAGGGCGGGAGGGCTTGGGAGACCCAGTACTTC |
| 1627 | 55-TL661-TIL-CD8+_CD103+ | TIL | CD8+_CD103+ | TGTGCCAGCAGCTTGGTTCAGGTGAACACTGAAGCTTTCTTT |
| 1628 | 55-TL661-TIL-CD8+_CD103+ | TIL | CD8+_CD103+ | TGTGCCAGCAGCTTGCGCTGGACAGGGCAGGATGGCTACACCTTC |
| 1629 | 55-TL661-TIL-CD8+_CD103+ | TIL | CD8+_CD103+ | TGTGCCAGCTCCTGGGACGGCACTGAAGCTTTCTTT |
| 1630 | 55-TL661-TIL-CD8+_CD103+ | TIL | CD8+_CD103+ | TGTGCCAGCAGCTCCCGAGGTACAATGAACACTGAAGCTTTCTTT |
| 1631 | 55-TL661-TIL-CD8+_CD103+ | TIL | CD8+_CD103+ | TGTGCCAGCAGCCAATCTACCCAGGGGTATTCACCCCTCCACTTT |
| 1632 | 55-TL661-TIL-CD8+_CD103+ | TIL | CD8+_CD103+ | TGCGCCAGCAGCTTGGCTCCTCTCTCAGGAGATAGACGTACAGAT |
| | | | | ACGCAGTATTTT |
| 1633 | 55-TL661-TIL-CD8+_CD103+ | TIL | CD8+_CD103+ | TGTAGCGGACACAAGAACACTGAAGCTTTCTTT |
| 1634 | 55-TL661-TIL-CD8+_CD103+ | TIL | CD8+_CD103+ | TGTGCCAGCAGCGAGGACGGCTCCTACGAGCAGTACTTC |
| 1635 | 55-TL661-TIL-CD8+_CD103+ | TIL | CD8+_CD103+ | TGCGCCAGCAGCCAAGTCGGGACAGTGAACACTGAAGCTTTCTTT |
| 1636 | 56-TL661-TIL-CD8+_CD103− | TIL | CD8+_CD103− | TGTGCCAGCAGCTTGGGTTACAATGAGCAGTTCTTC |
| 1637 | 56-TL661-TIL-CD8+_CD103− | TIL | CD8+_CD103− | TGCAGCGTCCTACTAGCGAACACAGATACGCAGTATTTT |
| 1638 | 56-TL661-TIL-CD8+_CD103− | TIL | CD8+_CD103− | TGTGCCAGCAGCCCAAGTGGATTTTCAACTAATGAAAAACTGTTTTTT |
| 1639 | 56-TL661-TIL-CD8+_CD103− | TIL | CD8+_CD103− | TGCAGTGCTAGAGATAGGAGCGGGAGAGAGTATTTT |
| 1640 | 56-TL661-TIL-CD8+_CD103− | TIL | CD8+_CD103− | TGCGCCAGCAGCTTGGCCGAGAGGGCAGGGGAAGAGACCCAGTACTTC |
| 1641 | 56-TL661-TIL-CD8+_CD103− | TIL | CD8+_CD103− | TGCGCCAGCAGCTGGGACAAGCCCTACGAGCAGTACTTC |
| 1642 | 56-TL661-TIL-CD8+_CD103− | TIL | CD8+_CD103− | TGTGCCAGCAGCTCAAATCCGAACACCGGGGAGCTGTTTTTT |
| 1643 | 56-TL661-TIL-CD8+_CD103− | TIL | CD8+_CD103− | TGCAGCGTTGACGAGACAGGGGACACTGAAGCTTTCTTT |
| 1644 | 56-TL661-TIL-CD8+_CD103− | TIL | CD8+_CD103− | TGTGCCAGCAGCTCCTCCGGGACTAACTATGGCTACACCTTC |
| 1645 | 56-TL661-TIL-CD8+_CD103− | TIL | CD8+_CD103− | TGCGCCAGCAGCCAATTCGGGGGAGACACTGAAGCTTTCTTT |
| 1646 | 56-TL661-TIL-CD8+_CD103− | TIL | CD8+_CD103− | TGTGCCAGCAGCTCCCTCTATGAGCAGTTCTTC |
| 1647 | 56-TL661-TIL-CD8+_CD103− | TIL | CD8+_CD103− | TGTGCCAGCAGTGATACCGGCAGCACCTCTTATGGCTACACCTTC |
| 1648 | 56-TL661-TIL-CD8+_CD103− | TIL | CD8+_CD103− | TGTGCCAGCAGCCCAGGGAAGGGAACTGAAGCTTTCTTT |
| 1649 | 56-TL661-TIL-CD8+_CD103− | TIL | CD8+_CD103− | TGCGCCAGCAGCTTGGCTCCTCTCTCAGGAGATAGACGT |
| | | | | ACAGATACGCAGTATTTT |
| 1650 | 56-TL661-TIL-CD8+_CD103− | TIL | CD8+_CD103− | TGTGCCAGCAGTTACCGGGGGGCCTACGAGCAGTACTTC |
| 1651 | 56-TL661-TIL-CD8+_CD103− | TIL | CD8+_CD103− | TGTGCCAGCAGCCATGGGGATCCCAATGAGCAGTTCTTC |
| 1652 | 56-TL661-TIL-CD8+_CD103− | TIL | CD8+_CD103− | TGCAGCGTAGCCCCCGGGACAGGGGGCTACGAGCAGTACTTC |
| 1653 | 56-TL661-TIL-CD8+_CD103− | TIL | CD8+_CD103− | TGTGCCAGCAGCTCCCTAGCGTCTAGCACAGATACGCAGTATTTT |
| 1654 | 56-TL661-TIL-CD8+_CD103− | TIL | CD8+_CD103− | TGTGCCAGCAGTGACACCCCGCAGGCAGCAGTCTATGGCTACACCTTC |
| 1655 | 56-TL661-TIL-CD8+_CD103− | TIL | CD8+_CD103− | TGTGCCAGTACCGGGGCGGACGGGGCCAACGTCCTGACTTTC |
| 1656 | 56-TL661-TIL-CD8+_CD103− | TIL | CD8+_CD103− | TGCAGCGTTGAAGACAGGGACAGCTCCACCGGGGAGCTGTTTTTT |
| 1657 | 56-TL661-TIL-CD8+_CD103− | TIL | CD8+_CD103− | TGCAGCGTTGACGACGGGGTTGATGAGCAGTACTTC |
| 1658 | 56-TL661-TIL-CD8+_CD103− | TIL | CD8+_CD103− | TGTGCCAGCAGTCCTTCCGGGAGAGGTGAGCAGTTCTTC |
| 1659 | 56-TL661-TIL-CD8+_CD103− | TIL | CD8+_CD103− | TGTGCCAGCAGCACCAGCCCAGGGGGCCGGGGCTACACCTTC |
| 1660 | 56-TL661-TIL-CD8+_CD103− | TIL | CD8+_CD103− | TGTGCCAGCAGTTCCTCGACAGGGATTCAGCCCCAGCATTTT |
| 1661 | 56-TL661-TIL-CD8+_CD103− | TIL | CD8+_CD103− | TGTGCCAGCAGCTTAGGCGGGGGCCTCGATGGCTACACCTTC |
| 1662 | 56-TL661-TIL-CD8+_CD103− | TIL | CD8+_CD103− | TGTGCCAGCGGTCAAGGGGGGGCTTGGGGCTACACCTTC |
| 1663 | 56-TL661-TIL-CD8+_CD103− | TIL | CD8+_CD103− | TGTGCCAGCAGCCAAGATAGAGCGGGAGGGATTTGGGAA |
| | | | | GAGACCCAGTACTTC |
| 1664 | 56-TL661-TIL-CD8+_CD103− | TIL | CD8+_CD103− | TGTGCCAGCAGCACCTCTAGGGAGGCCTCCTACAATGAGCAGTTCTTC |
| 1665 | 56-TL661-TIL-CD8+_CD103− | TIL | CD8+_CD103− | TGTGCCAGTAGTATCGGAAGGGGACTCTCCTACGAGCAGTACTTC |
| 1666 | 56-TL661-TIL-CD8+_CD103− | TIL | CD8+_CD103− | TGTGCCAGCAGCTGGGGCAGGGGCGCAAATTCACCCCTCCACTTT |
| 1667 | 56-TL661-TIL-CD8+_CD103− | TIL | CD8+_CD103− | TGTGCCAGCAGCTTGGGTCTAGCGGGGACCGGGGAGCTGTTTTTT |
| 1668 | 56-TL661-TIL-CD8+_CD103− | TIL | CD8+_CD103− | TGTGCCAGCAGCCCAAGTGGATTTTCAACTAATGAAAAACTGTTTTTT |
| 1669 | 56-TL661-TIL-CD8+_CD103− | TIL | CD8+_CD103− | TGCGCCAGCAGCCCCTCCGGGACGGCTAGCTCTGGAAACACCATATATTTT |
| 1670 | 56-TL661-TIL-CD8+_CD103− | TIL | CD8+_CD103− | TGTGCCACCTCTAGCGGTCCCTGGGGTGAGCAGTTCTTC |
| 1671 | 56-TL661-TIL-CD8+_CD103− | TIL | CD8+_CD103− | TGTGCCAGCAGCTTGGTTCAGGTGAACACTGAAGCTTTCTTT |
| 1672 | 56-TL661-TIL-CD8+_CD103− | TIL | CD8+_CD103− | TGTGCCAGCACGCTGGGGTCGGGGGGAGCTGAGCAGTTCTTC |
| 1673 | 56-TL661-TIL-CD8+_CD103− | TIL | CD8+_CD103− | TGCGCCAGCAGCCCCCACCTCGACAGCATTACTGAAGCTTTCTTT |
| 1674 | 56-TL661-TIL-CD8+_CD103− | TIL | CD8+_CD103− | TGTGCCAGCAGCGTCACTCGGGGGGGGACTGAAGCTTTCTTT |
| 1675 | 56-TL661-TIL-CD8+_CD103− | TIL | CD8+_CD103− | TGCGCCAGCAGCTTGGCCGGACGGAATAATATAGACACTGAAGCTTTCTTT |
| 1676 | 56-TL661-TIL-CD8+_CD103− | TIL | CD8+_CD103− | TGTGCCAGCAGTTCACAGGGTGACGCGGATCAGCCCCAGCATTTT |
| 1677 | 56-TL661-TIL-CD8+_CD103− | TIL | CD8+_CD103− | TGTGCCAGCAGTTTTTGGGGAGGCTACACCTTC |
| 1678 | 56-TL661-TIL-CD8+_CD103− | TIL | CD8+_CD103− | TGTGCCATCCGGGAGGACGGTAATAGCAATCAGCCCCAGCATTTT |
| 1679 | 56-TL661-TIL-CD8+_CD103− | TIL | CD8+_CD103− | TGTGCCAGCAGTCCTCTGGTCGACAGGGCTCAAGAGACCCAGTACTTC |
| 1680 | 56-TL661-TIL-CD8+_CD103− | TIL | CD8+_CD103− | TGTGCCAGCAGCGTTAGTTCTAAAGTGGCAGCCTACGAGCAGTACTTC |
| 1681 | 56-TL661-TIL-CD8+_CD103− | TIL | CD8+_CD103− | TGTGCCAGCAGTTTCAACAATGAGCAGTTCTTC |
| 1682 | 56-TL661-TIL-CD8+_CD103− | TIL | CD8+_CD103− | TGTGCCAGCAGTTATCTGGGGGTAACTGGGGCAGATACGCAGTATTTT |
| 1683 | 56-TL661-TIL-CD8+_CD103− | TIL | CD8+_CD103− | TGTGCCAGCAGCTTAGGGGGGACAGGGTCTGGCTACACCTTC |
| 1684 | 56-TL661-TIL-CD8+_CD103− | TIL | CD8+_CD103− | TGTGCCAGCAGTACGACTAGCGGGGGGGCCACAGATACGCAGTATTTT |
| 1685 | 56-TL661-TIL-CD8+_CD103− | TIL | CD8+_CD103− | TGTGCCATCAGTGAGGAAGGGACGCTCTACGAGCAGTACTTC |
| 1686 | 56-TL661-TIL-CD8+_CD103− | TIL | CD8+_CD103− | TGTGCCAGCAGCTTTACCGGGAGGGTCGACGAGCAGTACTTC |
| 1687 | 56-TL661-TIL-CD8+_CD103− | TIL | CD8+_CD103− | TGCGCCAGCAGCTTGGACTTGGCAGGCCTTGAAGCTTTCTTT |
| 1688 | 56-TL661-TIL-CD8+_CD103− | TIL | CD8+_CD103− | TGTGCCAGCAGTCCCCCGGGGGGAGGAGAGACCCAGTACTTC |
| 1689 | 56-TL661-TIL-CD8+_CD103− | TIL | CD8+_CD103− | TGTGCCAGCGGCGACGGAGGCACAGATACGCAGTATTTT |
| 1690 | 56-TL661-TIL-CD8+_CD103− | TIL | CD8+_CD103− | TGCAGCGTTGATCCGGAGGGATTGGAGACCCAGTACTTC |
| 1691 | 56-TL661-TIL-CD8+_CD103− | TIL | CD8+_CD103− | TGTGCCTGGAAAATGGCTGGAAACACCATATATTTT |
| 1692 | 56-TL661-TIL-CD8+_CD103− | TIL | CD8+_CD103− | TGTGCCAGCAGCAGAAGACTAGCGGAAGAGACCCAGTACTTC |
| 1693 | 56-TL661-TIL-CD8+_CD103− | TIL | CD8+_CD103− | TGTGCCAGCAGTCCTAGCGGGAGGGGTGAGCAGTTCTTC |
| 1694 | 56-TL661-TIL-CD8+_CD103− | TIL | CD8+_CD103− | TGTGCCAGTAGTATAGGTGGCAGCTACAATGAGCAGTTCTTC |
| 1695 | 56-TL661-TIL-CD8+_CD103− | TIL | CD8+_CD103− | TGTGCCAGTAGTTTTAAAGGTGGGGCCTACGAGCAGTACTTC |
| 1696 | 56-TL661-TIL-CD8+_CD103− | TIL | CD8+_CD103− | TGCGCCAGCAGCTTGAACGCGGGAGGGCCGCGGTTCTTC |
| 1697 | 56-TL661-TIL-CD8+_CD103− | TIL | CD8+_CD103− | TGCGCCAGCAGCCAATCGACTCCCAACCGGCATACGCAGTATTTT |
| 1698 | 56-TL661-TIL-CD8+_CD103− | TIL | CD8+_CD103− | TGTGCCAGCAGTTTGACAGGGGGCGAAGGCACTGAAGCTTTCTTT |
| 1699 | 56-TL661-TIL-CD8+_CD103− | TIL | CD8+_CD103− | TGTGCCTACAATGAGCAGTTCTTC |
| 1700 | 56-TL661-TIL-CD8+_CD103− | TIL | CD8+_CD103− | TGTGCCAGCAGTGCGGGGTCACTCGAAGTATCTAACTATGGCTACACCTTC |
| 1701 | 56-TL661-TIL-CD8+_CD103− | TIL | CD8+_CD103− | TGCGCCAGCAGCTTGGGGACTAGCGAGTTTCCCCCTCTT |
| | | | | CAAGAGACCCAGTACTTC |
| 1702 | 56-TL661-TIL-CD8+_CD103− | TIL | CD8+_CD103− | TGTGCCAGCAGCGTGACAGGGGGGACTGAAGCTTTCTTT |
| 1703 | 56-TL661-TIL-CD8+_CD103− | TIL | CD8+_CD103− | TGTGCCACCTTCGGGGGGGGGACCCAGTACTTC |
| 1704 | 56-TL661-TIL-CD8+_CD103− | TIL | CD8+_CD103− | TGTGCCACCAGCAGAGAGGAAGGGGATGGCTACACCTTC |
| 1705 | 56-TL661-TIL-CD8+_CD103− | TIL | CD8+_CD103− | TGTGCCAGCAGCCCCCCCGGGGCCTACGAGCAGTACTTC |
| 1706 | 56-TL661-TIL-CD8+_CD103− | TIL | CD8+_CD103− | TGCGCCAGCAGCTTGGCCTCGACAGACATGAACACTGAAGCTTTCTTT |
| 1707 | 56-TL661-TIL-CD8+_CD103− | TIL | CD8+_CD103− | TGTGCCAGCAGCTTAGCAACTAGCGGGCTTCACAATGAGCAGTTCTTC |
| 1708 | 56-TL661-TIL-CD8+_CD103− | TIL | CD8+_CD103− | TGCGCCAGCAGCCCCCAATGGACCCAGCATTTT |
| 1709 | 56-TL661-TIL-CD8+_CD103− | TIL | CD8+_CD103− | TGTGCCAGCCTACAGGGGGCGTATGGCTACACCTTC |
| 1710 | 56-TL661-TIL-CD8+_CD103− | TIL | CD8+_CD103− | TGTGCCAGCAGGCCTCCGGGGGGCGCGAGTACCCAGCCCCAGCATTTT |
| 1711 | 56-TL661-TIL-CD8+_CD103− | TIL | CD8+_CD103− | TGTGCCAGCAGCCAATCCGCCGGGGCACAGCCCCAGCATTTT |
| 1712 | 56-TL661-TIL-CD8+_CD103− | TIL | CD8+_CD103− | TGCGCCAGCAGGCCGAGCGGGGGGGCGTTGGATGGCTACACCTTC |
| 1713 | 56-TL661-TIL-CD8+_CD103− | TIL | CD8+_CD103− | TGTGCCAGCAGCGTAGATAGTCTAGCGGGACACGAGCAGTACTTC |
| 1714 | 56-TL661-TIL-CD8+_CD103− | TIL | CD8+_CD103− | TGTGCTAGTGGTTTGGACCCCTTGGGCACCGGGGAGCTGTTTTTT |
| 1715 | 56-TL661-TIL-CD8+_CD103− | TIL | CD8+_CD103− | TGTGCCAGCAGTTTAGGACGTCTGAATGAGCAGTTCTTC |
| 1716 | 56-TL661-TIL-CD8+_CD103− | TIL | CD8+_CD103− | TGTGCCAGCAGCCAAGATGCTTCCGGAGCTAACTATGGCTACACCTTC |
| 1717 | 56-TL661-TIL-CD8+_CD103− | TIL | CD8+_CD103− | TGCGCCAGCAGTGAGGATCAGGGGTTGAGTGAGCAGTTCTTC |
| 1718 | 56-TL661-TIL-CD8+_CD103− | TIL | CD8+_CD103− | TGTGCCACCAGCAGAGAAATCACGGGACAGGCTAATCAGCCCCAGCATTTT |
| 1719 | 56-TL661-TIL-CD8+_CD103− | TIL | CD8+_CD103− | TGTGCCAGCAGCCACCCGGGACAGGGGTACTTCTTC |
| 1720 | 56-TL661-TIL-CD8+_CD103− | TIL | CD8+_CD103− | TGTGCCAGCAGCACCTTTGCGGCGAACACCGGGGAGCTGTTTTTT |
| 1721 | 56-TL661-TIL-CD8+_CD103− | TIL | CD8+_CD103− | TGTGCCAGCAGTGAGGCTGAGGGGGGAGAAGAGCAGTACTTC |
| 1722 | 56-TL661-TIL-CD8+_CD103− | TIL | CD8+_CD103− | TGTGCCAGCAGTTTAGCAGGAGGCTCCTACAATGAGCAGTTCTTC |
| 1723 | 56-TL661-TIL-CD8+_CD103− | TIL | CD8+_CD103− | TGTGCCAGCAGCCCCGGCCCAGCGGGACCTTTCTTT |
| 1724 | 56-TL661-TIL-CD8+_CD103− | TIL | CD8+_CD103− | TGCGCCAGCAGCTTGGACATAAGGGGGACTGAAGCTTTCTTT |
| 1725 | 56-TL661-TIL-CD8+_CD103− | TIL | CD8+_CD103− | TGTGCCAGCAGTGCCCGAGTGTCCGGGCTCACTGAAGCTTTCTTT |
| 1726 | 56-TL661-TIL-CD8+_CD103− | TIL | CD8+_CD103− | TGCGCCAGCAGCTGGGCCCCGGGACAGCCCGACAATGAGCAGTTCTTC |
| 1727 | 56-TL661-TIL-CD8+_CD103− | TIL | CD8+_CD103− | TGTGCCAGCAGTCCGGGGACGGTTTCTTTCTACGAGCAGTACTTC |
| 1728 | 56-TL661-TIL-CD8+_CD103− | TIL | CD8+_CD103− | TGTGCCAGTTCAGGGGGCCGGACAGATACGCAGTATTTT |
| 1729 | 56-TL661-TIL-CD8+_CD103− | TIL | CD8+_CD103− | TGTGCCAGCAGCGTAGGGAGGCCCTATAATTCACCCCTCCACTTT |
| 1730 | 56-TL661-TIL-CD8+_CD103− | TIL | CD8+_CD103− | TGTGCCAGCAGGATCCCGGGGTTGTACGAGCAGTACTTC |
| 1731 | 56-TL661-TIL-CD8+_CD103− | TIL | CD8+_CD103− | TGTGCCAGCAGACGGCAGGGTACAGAGACCCAGTACTTC |
| 1732 | 56-TL661-TIL-CD8+_CD103− | TIL | CD8+_CD103− | TGTGCCAGCAGCGACAGGGATCTCTCTGGAAACACCATATATTTT |
| 1733 | 56-TL661-TIL-CD8+_CD103− | TIL | CD8+_CD103− | TGTGCCAGCAGCCAAGATATGACAGGGGGCGAGACCCAGTACTTC |
| 1734 | 56-TL661-TIL-CD8+_CD103− | TIL | CD8+_CD103− | TGTGCCAGCAGCCCCCGACGGAGGGAAGAGACCCAGTACTTC |
| 1735 | 56-TL661-TIL-CD8+_CD103− | TIL | CD8+_CD103− | TGTGCCAGCAGTTTATCAAATTCGGGCACTGAAGCTTTCTTT |
| 1736 | 56-TL661-TIL-CD8+_CD103− | TIL | CD8+_CD103− | TGCGCCAGCAGTGAGAATCCGGGAGTGGCAGATACGCAGTATTTT |
| 1737 | 56-TL661-TIL-CD8+_CD103− | TIL | CD8+_CD103− | TGCGCCAGCAAAGAGGGCACTGAAGCTTTCTTT |
| 1738 | 56-TL661-TIL-CD8+_CD103− | TIL | CD8+_CD103− | TGTGCCAGCAGTGCTACAGGTTCCGACTATGGCTACACCTTC |
| 1739 | 56-TL661-TIL-CD8+_CD103− | TIL | CD8+_CD103− | TGTGCCAGCGGGGGCAGGGGCTCTCAAGAGACCCAGTACTTC |
| 1740 | 56-TL661-TIL-CD8+_CD103− | TIL | CD8+_CD103− | TGTGCCAGCAGCTTAGTACAGAGCACAGATACGCAGTATTTT |
| 1741 | 56-TL661-TIL-CD8+_CD103− | TIL | CD8+_CD103− | TGCGCCAGCAGTGAGGGAGTTGGGGAGCTGTTTTTT |
| 1742 | 56-TL661-TIL-CD8+_CD103− | TIL | CD8+_CD103− | TGCGCCAGCAGCCAAGACAACTACAATGAGCAGTTCTTC |
| 1743 | 56-TL661-TIL-CD8+_CD103− | TIL | CD8+_CD103− | TGTGCCAGCAGTCCGCCCGCTTCAGGGGGCACTGAAGATACGCAGTATTTT |
| 1744 | 56-TL661-TIL-CD8+_CD103− | TIL | CD8+_CD103− | TGCGCCAGCACGGTGGGCTCCGGTGGAACCGGGGAGCTGTTTTTT |
| 1745 | 56-TL661-TIL-CD8+_CD103− | TIL | CD8+_CD103− | TGTGCCAGCAGTTTCGTGAGCTCCTACGAGCAGTACTTC |
| 1746 | 56-TL661-TIL-CD8+_CD103− | TIL | CD8+_CD103− | TGCGCCAGCAGTGAGTCGGGACAGGGATACGAGCAGTACTTC |
| 1747 | 63-TL663-TIL-CD8+_CD103+ | TIL | CD8+_CD103+ | TGTGCCAGCAGTACCCTCGATAGCAATCAGCCCCAGCATTTT |
| 1748 | 63-TL663-TIL-CD8+_CD103+ | TIL | CD8+_CD103+ | TGTGCCACCAGTGATCCCAGGGGAGCTGGGGCCAACGTCCTGACTTTC |
| 1749 | 63-TL663-TIL-CD8+_CD103+ | TIL | CD8+_CD103+ | TGTGCCAGCAGCCCTCCTAGGGCAGCGACGCAGTACTTC |
| 1750 | 63-TL663-TIL-CD8+_CD103+ | TIL | CD8+_CD103+ | TGTGCCAGCAGGGACTTGGACCGCTACGAGCAGTACTTC |
| 1751 | 63-TL663-TIL-CD8+_CD103+ | TIL | CD8+_CD103+ | TGTGCCAGCAGCTTGACAGGGTCAGGAGAGCAGTACTTC |
| 1752 | 63-TL663-TIL-CD8+_CD103+ | TIL | CD8+_CD103+ | TGTGCCAGCTGGGGGGAGGGAACTGAAGCTTTCTTT |
| 1753 | 63-TL663-TIL-CD8+_CD103+ | TIL | CD8+_CD103+ | TGTGCCAGCAGCTTAAGGGGTGCCGGGGAGCTGTTTTTT |
| 1754 | 63-TL663-TIL-CD8+_CD103+ | TIL | CD8+_CD103+ | TGTGCCAGCAGTCTACCAGCCGGGACAGGGGCCCTCACA |
| | | | | GATACGCAGTATTTT |
| 1755 | 63-TL663-TIL-CD8+_CD103+ | TIL | CD8+_CD103+ | TGTGCCAGCAGCCTCCTGGGGGGCCAAGATACGCAGTATTTT |
| 1756 | 63-TL663-TIL-CD8+_CD103+ | TIL | CD8+_CD103+ | TGTGCCAGCAGACAAGGGGATAGCAATCAGCCCCAGCATTTT |
| 1757 | 63-TL663-TIL-CD8+_CD103+ | TIL | CD8+_CD103+ | TGTGCCAGCAGCTTAGTCCCAGGGAGCTCCTACAATGAGCAGTTCTTC |
| 1758 | 63-TL663-TIL-CD8+_CD103+ | TIL | CD8+_CD103+ | TGTGCCACCAGTGATGTTACAGGGTCTGGGGCCAACGTCCTGACTTTC |
| 1759 | 63-TL663-TIL-CD8+_CD103+ | TIL | CD8+_CD103+ | TGTGCCAGCAGCGCAGGGAACACCGGGGAGCTGTTTTTT |
| 1760 | 63-TL663-TIL-CD8+_CD103+ | TIL | CD8+_CD103+ | TGTGCCAGCAGCCAAGAGGAAGGGAGTGGGGCCAACGTCCTGACTTTC |
| 1761 | 63-TL663-TIL-CD8+_CD103+ | TIL | CD8+_CD103+ | TGTGCCAGCAGCCAAGAGGGGGGTACTGGGGCCAACGTCCTGACTTTC |
| 1762 | 63-TL663-TIL-CD8+_CD103+ | TIL | CD8+_CD103+ | TGTGCCAGCAGCTTATCGGAGGGAGGGACAGATACGCAGTATTTT |
| 1763 | 63-TL663-TIL-CD8+_CD103+ | TIL | CD8+_CD103+ | TGTGCCAGCAGTGAGCCTGTTACGGGACTAGCGGGGCGGAGCTC |
| | | | | CTACAATGAGCAGTTCTTC |
| 1764 | 63-TL663-TIL-CD8+_CD103+ | TIL | CD8+_CD103+ | TGTGCCAGCAGCCGGACTAGCGGGGGCCCCAATGAGCAGTTCTTC |
| 1765 | 63-TL663-TIL-CD8+_CD103+ | TIL | CD8+_CD103+ | TGTGCCAGCAGTTTTGGAGGGGACGGCTACGAGCAGTACTTC |
| 1766 | 63-TL663-TIL-CD8+_CD103+ | TIL | CD8+_CD103+ | TGTGCCAGCAGCTTAGAGGCCCCTGGGCCCCAGCATTTT |
| 1767 | 63-TL663-TIL-CD8+_CD103+ | TIL | CD8+_CD103+ | TGTGCCAGCAGTTTAAGGAGGGACAGGGACGGAAACACCATATATTTT |
| 1768 | 63-TL663-TIL-CD8+_CD103+ | TIL | CD8+_CD103+ | TGTGCCAGCAGACTAGAGGACAGCATAAGCTCCGAGCAGTACTTC |
| 1769 | 63-TL663-TIL-CD8+_CD103+ | TIL | CD8+_CD103+ | TGTGCCAGCAGCTTGGGGGGGAACACTGAAGCTTTCTTT |
| 1770 | 63-TL663-TIL-CD8+_CD103+ | TIL | CD8+_CD103+ | TGTGCCAGCAGCTTATCCCCGGGGGCCAGCAATCAGCCCCAGCATTTT |
| 1771 | 63-TL663-TIL-CD8+_CD103+ | TIL | CD8+_CD103+ | TGTGCCAGCAGCTCCCCGGGACTAGCGTCGGAGACCCAGTACTTC |
| 1772 | 63-TL663-TIL-CD8+_CD103+ | TIL | CD8+_CD103+ | TGTGCCAGCAGTGATGGGACCCGCGACAATGGCTACACCTTC |
| 1773 | 63-TL663-TIL-CD8+_CD103+ | TIL | CD8+_CD103+ | TGTGCCAGCACCCGTTGGGCCCGACAACAGTTCTTC |
| 1774 | 63-TL663-TIL-CD8+_CD103+ | TIL | CD8+_CD103+ | TGTGCCAGCAGCTTGGAGGGACTAGCGGGGGGCCCTGCCCAC |
| | | | | AATGAGCAGTTCTTC |
| 1775 | 63-TL663-TIL-CD8+_CD103+ | TIL | CD8+_CD103+ | TGCGCCAGCAGCCCAAGTACTAGCAATGAGCAGTTCTTC |
| 1776 | 63-TL663-TIL-CD8+_CD103+ | TIL | CD8+_CD103+ | TGTGCCAGCAGTAGTGGCGGGGTAGCCTACAATGAGCAGTTCTTC |
| 1777 | 63-TL663-TIL-CD8+_CD103+ | TIL | CD8+_CD103+ | TGTGCCAGCAGCCTGGGGGGGGGAGCCAAAAACATTCAGTACTTC |
| 1778 | 63-TL663-TIL-CD8+_CD103+ | TIL | CD8+_CD103+ | TGTGCCAGCAGCTTAGCGCCTTGGCAAGCGGGAGAGGGGGAGCAGTACTTC |
| 1779 | 63-TL663-TIL-CD8+_CD103+ | TIL | CD8+_CD103+ | TGTGCCAGCAGCTGGACTAGCACCGGGGAGCTGTTTTTT |
| 1780 | 63-TL663-TIL-CD8+_CD103+ | TIL | CD8+_CD103+ | TGTGCCAGCAGCTTAGCGCCGGCTAGCGGGGGGGGCGCG |
| | | | | GATGAGCAGTTCTTC |
| 1781 | 63-TL663-TIL-CD8+_CD103+ | TIL | CD8+_CD103+ | TGTGCCAGCAGCAAAGGGGAGTTGGGGACACCCCCCCGG |
| | | | | GAGACCCAGTACTTC |
| 1782 | 63-TL663-TIL-CD8+_CD103+ | TIL | CD8+_CD103+ | TGTGCCAGCAGCCAGGGGCTGTCCTACGAGCAGTACTTC |
| 1783 | 63-TL663-TIL-CD8+_CD103+ | TIL | CD8+_CD103+ | TGTGCCAGCAGTCCGTCGGGACAGGACTACGAGCAGTACTTC |
| 1784 | 63-TL663-TIL-CD8+_CD103+ | TIL | CD8+_CD103+ | TGTGCCAGCAGCTCCGGGCCAAACTACGAGCAGTACTTC |
| 1785 | 63-TL663-TIL-CD8+_CD103+ | TIL | CD8+_CD103+ | TGTGCCAGCAGTTTCGTCGCGGGCACAGATACGCAGTATTTT |
| 1786 | 63-TL663-TIL-CD8+_CD103+ | TIL | CD8+_CD103+ | TGTGCCAGCAGCTTATATGGCGGCTACGAGCAGTACTTC |
| 1787 | 63-TL663-TIL-CD8+_CD103+ | TIL | CD8+_CD103+ | TGTGCCAGCAGTTCGACAGGGTGGGAGACCCAGTACTTC |
| 1788 | 63-TL663-TIL-CD8+_CD103+ | TIL | CD8+_CD103+ | TGTGCCAGCCTGCCCTACGGGATGGGCACAGATACGCAGTATTTT |
| 1789 | 63-TL663-TIL-CD8+_CD103+ | TIL | CD8+_CD103+ | TGTGCCAGCAGCCAAGAGACAGGCTCTGGGGCCAACGTCCTGACTTTC |
| 1790 | 63-TL663-TIL-CD8+_CD103+ | TIL | CD8+_CD103+ | TGTGCCAGCAGCGTCGGGACTGACTACGAGCAGTACTTC |
| 1791 | 63-TL663-TIL-CD8+_CD103+ | TIL | CD8+_CD103+ | TGTGCCAGCAGCTTTGGCACAAGGACTAGCGGGAGGTAC |
| | | | | TCGATCCAGTTCTTC |
| 1792 | 63-TL663-TIL-CD8+_CD103+ | TIL | CD8+_CD103+ | TGTGCCAGCAGCTCACTAGCGGGAGGGCCGTATGTCCCGAGTGA |
| | | | | GTACGAGCAGTACTTC |
| 1793 | 63-TL663-TIL-CD8+_CD103+ | TIL | CD8+_CD103+ | TGTGCCAGCAGCTTAGCTGTTGGGGTTACTAACTATGGCTACACCTTC |
| 1794 | 63-TL663-TIL-CD8+_CD103+ | TIL | CD8+_CD103+ | TGTGCCAGCAGTTTGGAGGGTCGGCAAGAGACCCAGTACTTC |
| 1795 | 63-TL663-TIL-CD8+_CD103+ | TIL | CD8+_CD103+ | TGTGCCAGCAGTGCGGGAGCCTACGAGCAGTACTTC |
| 1796 | 63-TL663-TIL-CD8+_CD103+ | TIL | CD8+_CD103+ | TGTGCCAGCAGCCAAGTTGGAGGGGGGGTTAATGAGCAGTTCTTC |
| 1797 | 63-TL663-TIL-CD8+_CD103+ | TIL | CD8+_CD103+ | TGTGCCAGCAGCCCCAGGGGCGGGACGGCCCGATACAATGAGCAGTTCTTC |
| 1798 | 63-TL663-TIL-CD8+_CD103+ | TIL | CD8+_CD103+ | TGCAGCGTTGAGAAGGAGGCAGGGGAGACCCAGTACTTC |
| 1799 | 63-TL663-TIL-CD8+_CD103+ | TIL | CD8+_CD103+ | TGCAGCGTTGAAGGTGTGTCAGTGAACACTGAAGCTTTCTTT |
| 1800 | 63-TL663-TIL-CD8+_CD103+ | TIL | CD8+_CD103+ | TGTGCCAGCAGTCATGGGACAGGAGATCCTAGTCGCTACGAGCAGTACTTC |
| 1801 | 63-TL663-TIL-CD8+_CD103+ | TIL | CD8+_CD103+ | TGTGCCTGGAGCCCCGGGGGACCGTACCGAAACGAGCAGTACTTC |
| 1802 | 63-TL663-TIL-CD8+_CD103+ | TIL | CD8+_CD103+ | TGTGCCAGTACTGCACAGGGATCGAACACTGAAGCTTTCTTT |
| 1803 | 63-TL663-TIL-CD8+_CD103+ | TIL | CD8+_CD103+ | TGCGCCAGCACCCCAGGGATCAACACCGGGGAGCTGTTTTTT |
| 1804 | 63-TL663-TIL-CD8+_CD103+ | TIL | CD8+_CD103+ | TGTGCCAGCAGGGACAGGGTCGGGGAGCTGTTTTTT |
| 1805 | 63-TL663-TIL-CD8+_CD103+ | TIL | CD8+_CD103+ | TGTGCCAGCAGCCAGGTTGGGAGATACGAGCAGTACTTC |
| 1806 | 63-TL663-TIL-CD8+_CD103+ | TIL | CD8+_CD103+ | TGCGCCAGCAGCTTGGGTGGCACTGAAGCTTTCTTT |
| 1807 | 63-TL663-TIL-CD8+_CD103+ | TIL | CD8+_CD103+ | TGCAGTGCTAGAGATCCTACGCGGGGTGGGAGCTCCTACGAGCAGTACTTC |
| 1808 | 63-TL663-TIL-CD8+_CD103+ | TIL | CD8+_CD103+ | TGTGCCAGCGAGAGACCGAACACCGGGGAGCTGTTTTTT |
| 1809 | 63-TL663-TIL-CD8+_CD103+ | TIL | CD8+_CD103+ | TGTGCCAGCAGTTACTTTGGGGACACTGAAGCTTTCTTT |
| 1810 | 63-TL663-TIL-CD8+_CD103+ | TIL | CD8+_CD103+ | TGTGCCAGCAGCTTAGCGGGGACAGCCTATGGCTACACCTTC |
| 1811 | 63-TL663-TIL-CD8+_CD103+ | TIL | CD8+_CD103+ | TGTGCCAGCAGTTTATGGCAGGGAACGAACACCGGGGAGCTGTTTTTT |
| 1812 | 63-TL663-TIL-CD8+_CD103+ | TIL | CD8+_CD103+ | TGTGCCACCAGTGGGAGGGACAGGGGGTCAGATACGCAGTATTTT |
| 1813 | 63-TL663-TIL-CD8+_CD103+ | TIL | CD8+_CD103+ | TGTGCCAGCAGCTTACTACGAGGAAAACTGTTTTTT |
| 1814 | 63-TL663-TIL-CD8+_CD103+ | TIL | CD8+_CD103+ | TGTGCCAGCAGCTCGGGACCGAACTACGAGCAGTACTTC |
| 1815 | 63-TL663-TIL-CD8+_CD103+ | TIL | CD8+_CD103+ | TGCGCCAGCAGCCAGACTGGGACAGGGAACACTGAAGCTTTCTTT |
| 1816 | 63-TL663-TIL-CD8+_CD103+ | TIL | CD8+_CD103+ | TGTGCCAGCAGTTACCAGGGAGGAGACTATGGCTACACCTTC |
| 1817 | 63-TL663-TIL-CD8+_CD103+ | TIL | CD8+_CD103+ | TGTGCCAGCAGCTTATGGTCGCTAGCGGCCAAAGAGCCCCAGTACTTC |
| 1818 | 63-TL663-TIL-CD8+_CD103+ | TIL | CD8+_CD103+ | TGTGCCAGCAGATTTCACCACCGGGGGAGCTACGAGCAGTACTTC |
| 1819 | 63-TL663-TIL-CD8+_CD103+ | TIL | CD8+_CD103+ | TGCAGTGCTAGACTAGCGGGAGCCTTAAGGTTCGAGCAGTTCTTC |
| 1820 | 63-TL663-TIL-CD8+_CD103+ | TIL | CD8+_CD103+ | TGTGCCACCAGCAGCACAGACTGGGGGACTGAAGCTTTCTTT |
| 1821 | 63-TL663-TIL-CD8+_CD103+ | TIL | CD8+_CD103+ | TGTGCCACCAGACCGGGACAGGGTTTTAATGAGCAGTTCTTC |
| 1822 | 63-TL663-TIL-CD8+_CD103+ | TIL | CD8+_CD103+ | TGCAGTGCTCCTTACGAGACGTGGGCGAAGATCGAGAACACT |
| | | | | GAAGCTTTCTTT |
| 1823 | 63-TL663-TIL-CD8+_CD103+ | TIL | CD8+_CD103+ | TGTGCCAGTACCGCGACAGGGGATGGCTACACCTTC |
| 1824 | 63-TL663-TIL-CD8+_CD103+ | TIL | CD8+_CD103+ | TGTGCCTGGAGGAGCCGGACAGGGTGGCACGAGCAGTACTTC |
| 1825 | 63-TL663-TIL-CD8+_CD103+ | TIL | CD8+_CD103+ | TGTGCCAGCAGTACCCTCGATAGCAATCAGCCACAGCATTTT |
| 1826 | 63-TL663-TIL-CD8+_CD103+ | TIL | CD8+_CD103+ | TGCAGCGCCCGGGAAGGGGCCTACGAGCAGTACTTC |
| 1827 | 63-TL663-TIL-CD8+_CD103+ | TIL | CD8+_CD103+ | TGTGCCTGGAGTACCCGGGACACCTACGAGCAGTACTTC |
| 1828 | 63-TL663-TIL-CD8+_CD103+ | TIL | CD8+_CD103+ | TGCAGTGCGTTCGGGACAGTTGATCAGCCCCAGCATTTT |
| 1829 | 63-TL663-TIL-CD8+_CD103+ | TIL | CD8+_CD103+ | TGTGCCAGCAGCGTAGGGACAGATTACGAGCAGTACTTC |
| 1830 | 63-TL663-TIL-CD8+_CD103+ | TIL | CD8+_CD103+ | TGTGCCAGATCTCCTTCCTCCGGGACAGTAATATCTTAC |
| | | | | AATGAGCAGTTCTTC |
| 1831 | 63-TL663-TIL-CD8+_CD103+ | TIL | CD8+_CD103+ | TGCGCCAGCAGCCACGTCGGGACTAGCGGTTACGAGCAGTACTTC |
| 1832 | 63-TL663-TIL-CD8+_CD103+ | TIL | CD8+_CD103+ | TGTGCCAGCAGCCAAGATCCGCACGGGGCCAGGAACGAGCAGTACTTC |
| 1833 | 63-TL663-TIL-CD8+_CD103+ | TIL | CD8+_CD103+ | TGCAGCGTTGTGACAGAGAACACTGAAGCTTTCTTT |
| 1834 | 63-TL663-TIL-CD8+_CD103+ | TIL | CD8+_CD103+ | TGTGCCATCAGTGAGTCGTACGGACCAAAACAAGAGACCCAGTACTTC |
| 1835 | 63-TL663-TIL-CD8+_CD103+ | TIL | CD8+_CD103+ | TGTGCCAGCAGTTTACCCCCACACAATGAGCAGTTCTTC |
| 1836 | 63-TL663-TIL-CD8+_CD103+ | TIL | CD8+_CD103+ | TGTGCCAGCAGCACCAGCCCAGGGGGCCGGGGCTACACCTTC |
| 1837 | 63-TL663-TIL-CD8+_CD103+ | TIL | CD8+_CD103+ | TGTGCCAGCAGCTTAAGAGGGAACACTGAAGCTTTCTTT |
| 1838 | 63-TL663-TIL-CD8+_CD103+ | TIL | CD8+_CD103+ | TGTGCCAGCAGTCCCCCGGGACTAGCGGGGTCCTACGAGCAGTACTTC |
| 1839 | 63-TL663-TIL-CD8+_CD103+ | TIL | CD8+_CD103+ | TGTGCCAGCAGTGCGGGGACGGTTGGAACTGAAGCTTTCTTT |
| 1840 | 63-TL663-TIL-CD8+_CD103+ | TIL | CD8+_CD103+ | TGCGCCAGCACCCTGTCCCCGGGACAGGGGGCCTCCGGGGAGCTGTTTTTT |
| 1841 | 63-TL663-TIL-CD8+_CD103+ | TIL | CD8+_CD103+ | TGCAGCGTTGCTGACAATGAGCAGTTCTTC |
| 1842 | 63-TL663-TIL-CD8+_CD103+ | TIL | CD8+_CD103+ | TGTGCCAGCAGCTCCGGGGTAGCGGGAGAATTTTACGAGCAGTACTTC |
| 1843 | 63-TL663-TIL-CD8+_CD103+ | TIL | CD8+_CD103+ | TGTGCCAGCAGTTTTCGGCCCCCTCCCTACGAGCAGTACTTC |
| 1844 | 63-TL663-TIL-CD8+_CD103+ | TIL | CD8+_CD103+ | TGCGCCAGCAGCCAAGGAGCGGGAGCCCCCGTTGAGCAGTTCTTC |
| 1845 | 63-TL663-TIL-CD8+_CD103+ | TIL | CD8+_CD103+ | TGTGCCAGCAGCTGGGGGGCTCAGCCCCAGCATTTT |
| 1846 | 63-TL663-TIL-CD8+_CD103+ | TIL | CD8+_CD103+ | TGTGCCACCAGCACAGACGGACAGGGTATAGACATTCAGTACTTC |
| 1847 | 63-TL663-TIL-CD8+_CD103+ | TIL | CD8+_CD103+ | TGTGCCAGCAGTGAGGGACAGGCCTTGTACACCGGGGAGCTGTTTTTT |
| 1848 | 63-TL663-TIL-CD8+_CD103+ | TIL | CD8+_CD103+ | TGCAGCGTCACAAGAGATACGCAGTATTTT |
| 1849 | 63-TL663-TIL-CD8+_CD103+ | TIL | CD8+_CD103+ | TGTGCCAGCAGCTCCGATATGGGAATGAGGGAGAGCACA |
| | | | | GATACGCAGTATTTT |
| 1850 | 63-TL663-TIL-CD8+_CD103+ | TIL | CD8+_CD103+ | TGCAGTGCTAGGCCGGTGGGGAACACTGAAGCTTTCTTT |
| 1851 | 63-TL663-TIL-CD8+_CD103+ | TIL | CD8+_CD103+ | TGTGCCAGCAGTACGACCCCGGGACAGGGTACAAACTATGGCTACACCTTC |
| 1852 | 63-TL663-TIL-CD8+_CD103+ | TIL | CD8+_CD103+ | TGTGCCAGCAGCTCGCTTACTAGCGGTACGAACACCGGGGAGCTGTTTTTT |
| 1853 | 63-TL663-TIL-CD8+_CD103+ | TIL | CD8+_CD103+ | TGTGCCAGCAGCTTAGGGTCGACAGGCGAAAAACTGTTTTTT |
| 1854 | 63-TL663-TIL-CD8+_CD103+ | TIL | CD8+_CD103+ | TGTGCCAGCAGCCCCGTAGCGGGAGGGTTGTTGTATGAGCAGTTCTTC |
| 1855 | 63-TL663-TIL-CD8+_CD103+ | TIL | CD8+_CD103+ | TGTGCCAGCAGTTACGGCAGCAAGCAAGAGACCCAGTACTTC |
| 1856 | 63-TL663-TIL-CD8+_CD103+ | TIL | CD8+_CD103+ | TGTGCCAGCAGCTTGGGAGTGGAGAATGAGCAGTTCTTC |
| 1857 | 63-TL663-TIL-CD8+_CD103+ | TIL | CD8+_CD103+ | TGTGCCAGCAGTCTTACGGATGGTCAAGAGACCCAGTACTTC |
| 1858 | 63-TL663-TIL-CD8+_CD103+ | TIL | CD8+_CD103+ | TGCAGTGCTAGTGATGTAGCGGGAGGTTACGAGCAGTACTTC |
| 1859 | 63-TL663-TIL-CD8+_CD103+ | TIL | CD8+_CD103+ | TGCAGCGTCGTCAGTCCCGGCTACGAGCAGTACTTC |
| 1860 | 63-TL663-TIL-CD8+_CD103+ | TIL | CD8+_CD103+ | TGTGCCAGCAGCTTGTTCCGGGGTACCGGGGAGCTGTTTTTT |
| 1861 | 63-TL663-TIL-CD8+_CD103+ | TIL | CD8+_CD103+ | TGTGCCAGCAGCTTAGCAGCGGCGATGAACACTGAAGCTTTCTTT |
| 1862 | 63-TL663-TIL-CD8+_CD103+ | TIL | CD8+_CD103+ | TGTGCCAGCAAGAGCAGGGCCGGCAGTCCCTACGAGCAGTACTTC |
| 1863 | 63-TL663-TIL-CD8+_CD103+ | TIL | CD8+_CD103+ | TGTGCCAGCAGTTACGGGGCCTCATACGAGCAGTACTTC |
| 1864 | 63-TL663-TIL-CD8+_CD103+ | TIL | CD8+_CD103+ | TGCAGCGTTGCCCCGACAGAACTTAACTATGGCTACACCTTC |
| 1865 | 63-TL663-TIL-CD8+_CD103+ | TIL | CD8+_CD103+ | TGTGCCAGTAGTATAGTGCAATTCTACGAGCAGTACTTC |
| 1866 | 63-TL663-TIL-CD8+_CD103+ | TIL | CD8+_CD103+ | TGCGCCAGCAGCCAAGATCTAGGGATGCACAATCAGCCCCAGCATTTT |
| 1867 | 63-TL663-TIL-CD8+_CD103+ | TIL | CD8+_CD103+ | TGTGCCAGCAGTTCACTAGCGGGGACCTTGTACCAAGAGACCCAGTACTTC |
| 1868 | 63-TL663-TIL-CD8+_CD103+ | TIL | CD8+_CD103+ | TGTGCCAGCAGTCCATGGGACAGGGGATTGCAAGATACGCAGTATTTT |
| 1869 | 63-TL663-TIL-CD8+_CD103+ | TIL | CD8+_CD103+ | TGTGCCATCAGTGAGTCGATAGGGTACGAGCAGTACTTC |
| 1870 | 63-TL663-TIL-CD8+_CD103+ | TIL | CD8+_CD103+ | TGTGCCACCAGCAGAGATCGGGTTGGACAGGCGAACGGGGAGCTGTTTTTT |
| 1871 | 63-TL663-TIL-CD8+_CD103+ | TIL | CD8+_CD103+ | TGTGCCAGCAGCTTCAGAACTAGCAACGCGCAGTATTTT |
| 1872 | 63-TL663-TIL-CD8+_CD103+ | TIL | CD8+_CD103+ | TGTGCCAGCAGTTTGGATAGTAAGGGCCCTCCTCGCGACGAGCAGTACTTC |
| 1873 | 63-TL663-TIL-CD8+_CD103+ | TIL | CD8+_CD103+ | TGTGCCAGTAGTATGGATCGTGCTAGCACAGATACGCAGTATTTT |
| 1874 | 63-TL663-TIL-CD8+_CD103+ | TIL | CD8+_CD103+ | TGTGCCAGCAGCTTGGCCCAGGGGGCGGACACTGAAGCTTTCTTT |
| 1875 | 63-TL663-TIL-CD8+_CD103+ | TIL | CD8+_CD103+ | TGTGCCAGCAGAGCCTACAGGGAGCTGGGCACTGAAGCTTTCTTT |
| 1876 | 63-TL663-TIL-CD8+_CD103+ | TIL | CD8+_CD103+ | TGTGCCAGCAGTTACCCGGCAACTAATGAAAAACTGTTTTTT |
| 1877 | 63-TL663-TIL-CD8+_CD103+ | TIL | CD8+_CD103+ | TGCGCCAGCAGCTTGGTCGGGGGCCGGGAGACCCAGTACTTC |
| 1878 | 63-TL663-TIL-CD8+_CD103+ | TIL | CD8+_CD103+ | TGTGCCAGCAGCCCCTCCGGGACAGGGGGCGAGGAGACCCAGTACTTC |
| 1879 | 63-TL663-TIL-CD8+_CD103+ | TIL | CD8+_CD103+ | TGTGCCAGCAGTTACGGCAGCAAGCAAGAGACCCAGTACTTC |
| 1880 | 63-TL663-TIL-CD8+_CD103+ | TIL | CD8+_CD103+ | TGTGCCAGCAGCCAAGCACAGGGGGCTGGTAATTCACCCCTCCACTTT |
| 1881 | 63-TL663-TIL-CD8+_CD103+ | TIL | CD8+_CD103+ | TGTGCCAGCGGGCCTACTAGTGACTCCTACAATGAGCAGTTCTTC |
| 1882 | 63-TL663-TIL-CD8+_CD103+ | TIL | CD8+_CD103+ | TGTGCCAGCAGCGACCGGACAGCGACAGATACGCAGTATTTT |
| 1883 | 63-TL663-TIL-CD8+_CD103+ | TIL | CD8+_CD103+ | TGTGCCAGCAGCCTACATCAGGGACCTTCCTACGAGCAGTACTTC |
| 1884 | 63-TL663-TIL-CD8+_CD103+ | TIL | CD8+_CD103+ | TGTGCCAGCAGTTACTCGAGGTCAATTAACACCGGGGAGCTGTTTTTT |
| 1885 | 63-TL663-TIL-CD8+_CD103+ | TIL | CD8+_CD103+ | TGTGCCAGCAGCACCATACAGGAGCCGAACACTGAAGCTTTCTTT |
| 1886 | 63-TL663-TIL-CD8+_CD103+ | TIL | CD8+_CD103+ | TGCAGCGTTGAAGCAGAAGGTGGCTACACCTTC |
| 1887 | 63-TL663-TIL-CD8+_CD103+ | TIL | CD8+_CD103+ | TGTGCCAGCAGTGAAGTTGTCGGAGGGCTCGAGCAGTACTTC |
| 1888 | 63-TL663-TIL-CD8+_CD103+ | TIL | CD8+_CD103+ | TGCAGCGTTGAAGAGTTACCAGGAGGGAACACTGAAGCTTTCTTT |
| 1889 | 63-TL663-TIL-CD8+_CD103+ | TIL | CD8+_CD103+ | TGTGCCACCAGCTATTACAGGGGAAAAGAGACCCAGTACTTC |
| 1890 | 63-TL663-TIL-CD8+_CD103+ | TIL | CD8+_CD103+ | TGTGCCAGCAAAATGGAGGGAGGGCCCTCCTACGAGCAGTACTTC |
| 1891 | 63-TL663-TIL-CD8+_CD103+ | TIL | CD8+_CD103+ | TGTGCCAGCAGCTTTGGGGGGGATCAGCCCCAGCATTTT |
| 1892 | 63-TL663-TIL-CD8+_CD103+ | TIL | CD8+_CD103+ | TGTGCCAGCAGCGCGACCGCTGCAGGTAATCAGCCCCAGCATTTT |
| 1893 | 63-TL663-TIL-CD8+_CD103+ | TIL | CD8+_CD103+ | TGTGCCAGCAGCCCGGGGACAGGGGTGGAAGCTTTCTTT |
| 1894 | 63-TL663-TIL-CD8+_CD103+ | TIL | CD8+_CD103+ | TGTGCCAGCAGGGGACAGGGATATTCCTACGAGCAGTACTTC |
| 1895 | 63-TL663-TIL-CD8+_CD103+ | TIL | CD8+_CD103+ | TGTGCCAGCAGCCTCGGGACTCCTACCTACGAGCAGTACTTC |
| 1896 | 63-TL663-TIL-CD8+_CD103+ | TIL | CD8+_CD103+ | TGTGCCAGCAGCTTAAGTATTAGCCATGAGCAGTTCTTC |
| 1897 | 63-TL663-TIL-CD8+_CD103+ | TIL | CD8+_CD103+ | TGTGCCAGTAGTATCGGTGCGGGAGCCCCGTTTGACATTCAGTACTTC |
| 1898 | 63-TL663-TIL-CD8+_CD103+ | TIL | CD8+_CD103+ | TGTGCCAGCAGCTATTCTTATAGCACAGATACGCAGTATTTT |
| 1899 | 63-TL663-TIL-CD8+_CD103+ | TIL | CD8+_CD103+ | TGTGCCAGCACAGCGGGACAGGGGGCTCGTGGAAACACCATATATTTT |
| 1900 | 63-TL663-TIL-CD8+_CD103+ | TIL | CD8+_CD103+ | TGCAGTGCACCCCTTAGCGGGGGGTTGTACAATGAGCAGTTCTTC |
| 1901 | 63-TL663-TIL-CD8+_CD103+ | TIL | CD8+_CD103+ | TGTGCCAGCAGTTTCCATTCCGGCTTTTACGAGCAGTACTTC |
| 1902 | 63-TL663-TIL-CD8+_CD103+ | TIL | CD8+_CD103+ | TGCGCCAGCAGCCAAGGGAGGGGCGCACCCACTGAAGCUTCUT |
| 1903 | 63-TL663-TIL-CD8+_CD103+ | TIL | CD8+_CD103+ | TGTGCCAGCAGTTTAGCCGCCGACTGGAAGTCCTACGAGCAGTACTTC |
| 1904 | 63-TL663-TIL-CD8+_CD103+ | TIL | CD8+_CD103+ | TGTGCCAGCAGCTTAGGACTAGCGGCTGGCAATGAGCAGTTCTTC |
| 1905 | 63-TL663-TIL-CD8+_CD103+ | TIL | CD8+_CD103+ | TGTGCCAGCAGCCCGGGGACAGATACGCAGTATTTT |
| 1906 | 63-TL663-TIL-CD8+_CD103+ | TIL | CD8+_CD103+ | TGTGCCAGTAGTATAGGACACGACGGCATGAACACTGAAGCUTCUT |
| 1907 | 63-TL663-TIL-CD8+_CD103+ | TIL | CD8+_CD103+ | TGTGCCAGCGGCTGGACAGGGCCTGAGACCCAGTACTTC |
| 1908 | 63-TL663-TIL-CD8+_CD103+ | TIL | CD8+_CD103+ | TGTGCCAGCAGCCCTGGGACAGCTCTCGAGCAGTACTTC |
| 1909 | 63-TL663-TIL-CD8+_CD103+ | TIL | CD8+_CD103+ | TGTGCCACCAGTGATCCCAGGGGAGCTGGGGCCAACGTCTTGACTTTC |
| 1910 | 64-TL663-TIL-CD8+_CD103− | TIL | CD8+_CD103− | TGTGCCAGCAGCTCACAGGGCAATAAGATCGAGCAGTACTTC |
| 1911 | 64-TL663-TIL-CD8+_CD103− | TIL | CD8+_CD103− | TGTGCCAGCAGCCAAGCACAGGGGGCTGGTAATTCACCCCTCCACTTT |
| 1912 | 64-TL663-TIL-CD8+_CD103− | TIL | CD8+_CD103− | TGTGCCAGCAGTGTCGTGGGCTCGAGCTACGAGCAGTACTTC |
| 1913 | 64-TL663-TIL-CD8+_CD103− | TIL | CD8+_CD103− | TGTGCCAGCAGCTCGCAGCAAGCCAAAAACATTCAGTACTTC |
| 1914 | 64-TL663-TIL-CD8+_CD103− | TIL | CD8+_CD103− | TGTGCCAGCAGTTTATCGTATGGGGGAAATTCACCCCTCCACTTT |
| 1915 | 64-TL663-TIL-CD8+_CD103− | TIL | CD8+_CD103− | TGTGCCAGCAGTCATGGGACAGGAGATCCTAGTCGCTACGAGCAGTACTTC |
| 1916 | 64-TL663-TIL-CD8+_CD103− | TIL | CD8+_CD103− | TGTGCCAGCAGCCTTGGTAGTTGGAGCACCGGGGAGCTGTTTTTT |
| 1917 | 64-TL663-TIL-CD8+_CD103− | TIL | CD8+_CD103− | TGTGCCAGCAGCGCGACCGCTGCAGGTAATCAGCCCCAGCATTTT |
| 1918 | 64-TL663-TIL-CD8+_CD103− | TIL | CD8+_CD103− | TGTGCCAGCAGTTTTTCAGGGGACTCCTACAATGAGCAGTTCTTC |
| 1919 | 64-TL663-TIL-CD8+_CD103− | TIL | CD8+_CD103− | TGTGCCAGCAGCTCCGGGCCAAACTACGAGCAGTACTTC |
| 1920 | 64-TL663-TIL-CD8+_CD103− | TIL | CD8+_CD103− | TGTGCCAGCAGCTTAAGCTGGGAGAACACTGAAGCTTTCTTT |
| 1921 | 64-TL663-TIL-CD8+_CD103− | TIL | CD8+_CD103− | TGTGCCAGCAGCTACCACTTTCAGGTGGACACCGGGGAGCTGTTTTTT |
| 1922 | 64-TL663-TIL-CD8+_CD103− | TIL | CD8+_CD103− | TGTGCCAGCAGTCTACCAGCCGGGACAGGGGCCCTCACAGATACGCAG |
| | | | | TATTTT |
| 1923 | 64-TL663-TIL-CD8+_CD103− | TIL | CD8+_CD103− | TGTGCCAGCAGCTTGGGGGGGAACACTGAAGCTTTCTTT |
| 1924 | 64-TL663-TIL-CD8+_CD103− | TIL | CD8+_CD103− | TGCGCCAGCATGTGGGGGCCCCGGAGGGGCACTGAAGCTTTCTTT |
| 1925 | 64-TL663-TIL-CD8+_CD103− | TIL | CD8+_CD103− | TGTGCCAGCAGTTCCGGGACAGCTTACAATCAGCCCCAGCATTTT |
| 1926 | 64-TL663-TIL-CD8+_CD103− | TIL | CD8+_CD103− | TGTGCCAGCAGCATCGGACAGGGCCCTTCCTACGAGCAGTACTTC |
| 1927 | 64-TL663-TIL-CD8+_CD103− | TIL | CD8+_CD103− | TGTGCCAGCAGCTTAGGTGGGGGCATGGGGGAGCAGTACTTC |
| 1928 | 64-TL663-TIL-CD8+_CD103− | TIL | CD8+_CD103− | TGTGCCAGCAAGACAGGGGGTAGCACAGATACGCAGTATTTT |
| 1929 | 64-TL663-TIL-CD8+_CD103− | TIL | CD8+_CD103− | TGTGCCAGCAGCTTATCCCCGGGGGCCAGCAATCAGCCCCAGCATTTT |
| 1930 | 64-TL663-TIL-CD8+_CD103− | TIL | CD8+_CD103− | TGTGCCAGCAGCAGTCCCATCTCCTACGAGCAGTACTTC |
| 1931 | 64-TL663-TIL-CD8+_CD103− | TIL | CD8+_CD103− | TGTGCCAGCAGTTCCCCGGACACCTACGGCGGGGAGCTGTTTTTT |
| 1932 | 64-TL663-TIL-CD8+_CD103− | TIL | CD8+_CD103− | TGTGCCAGCAGCTGGACTAGCACCGGGGAGCTGTTTTTT |
| 1933 | 64-TL663-TIL-CD8+_CD103− | TIL | CD8+_CD103− | TGTGCCAGTAGTAAACAGGGGGCGACCACTGAAGCTTTCTTT |
| 1934 | 64-TL663-TIL-CD8+_CD103− | TIL | CD8+_CD103− | TGTGCCAGCAGCACCGCAGGGGGAGGCGTAACCCAGTACTTC |
| 1935 | 64-TL663-TIL-CD8+_CD103− | TIL | CD8+_CD103− | TGTGCCAGCAGCTTAAGGCAGGGGCCGTCCTACGAGCAGTACTTC |
| 1936 | 64-TL663-TIL-CD8+_CD103− | TIL | CD8+_CD103− | TGTGCCAGCAGCCCCAGACAGGGGGCTTTGAATGAGCAGTTCTTC |
| 1937 | 64-TL663-TIL-CD8+_CD103− | TIL | CD8+_CD103− | TGTGCCAGCAGCTTACGGGGGAACACTGAAGCTTTCTTT |
| 1938 | 64-TL663-TIL-CD8+_CD103− | TIL | CD8+_CD103− | TGTGCCAGCAGCTTATCGGAGGGAGGGACAGATACGCAGTATTTT |
| 1939 | 64-TL663-TIL-CD8+_CD103− | TIL | CD8+_CD103− | TGTGCCAGCAGTCCCCACCGCTACGAGCAGTACTTC |
| 1940 | 64-TL663-TIL-CD8+_CD103− | TIL | CD8+_CD103− | TGTGCCAGCAGCTTAAGGGGAAATTCACCCCTCCACTTT |
| 1941 | 64-TL663-TIL-CD8+_CD103− | TIL | CD8+_CD103− | TGTGCCAGCAGTGACAGGGTACGAAGCGGGGAGCTGTTTTTT |
| 1942 | 64-TL663-TIL-CD8+_CD103− | TIL | CD8+_CD103− | TGTGCCAGCAGCCAAGATCCGCACGGGGCCAGGAACGAGCAGTACTTC |
| 1943 | 64-TL663-TIL-CD8+_CD103− | TIL | CD8+_CD103− | TGTGCCAGCAGCGCAGGAACCCCCGGGGCTTTCGAGCAGTACTTC |
| 1944 | 64-TL663-TIL-CD8+_CD103− | TIL | CD8+_CD103− | TGCGCCAGCAGCCAAGGAGCGGGAGCCCCCGTTGAGCAGTTCTTC |
| 1945 | 64-TL663-TIL-CD8+_CD103− | TIL | CD8+_CD103− | TGTGCCAGCAGTCTCCGGAACGGATATAAACTGTTTTTT |
| 1946 | 64-TL663-TIL-CD8+_CD103− | TIL | CD8+_CD103− | TGCGCCAGCAGCCAAGATTACCTAGCGGGGGGCCGGGCTGAGCAGTTCTTC |
| 1947 | 64-TL663-TIL-CD8+_CD103− | TIL | CD8+_CD103− | TGCAGTGCTGGCCGACAGGGCCGTAGCAATCAGCCCCAGCATTTT |
| 1948 | 64-TL663-TIL-CD8+_CD103− | TIL | CD8+_CD103− | TGTGCCACCAGCAGAGACGGGCCGGAGCAGTTCTTC |
| 1949 | 64-TL663-TIL-CD8+_CD103− | TIL | CD8+_CD103− | TGCAGTGCTAATGTGGGACCAAATAATTCACCCCTCCACTTT |
| 1950 | 64-TL663-TIL-CD8+_CD103− | TIL | CD8+_CD103− | TGTGCCAGCAGGTTGGGACAGGGGGACTATGGCTACACCTTC |
| 1951 | 64-TL663-TIL-CD8+_CD103− | TIL | CD8+_CD103− | TGTGCCAGCAGTGAAGTTGTCGGAGGGCTCGAGCAGTACTTC |
| 1952 | 64-TL663-TIL-CD8+_CD103− | TIL | CD8+_CD103− | TGTGCCAGCAGCCCCACCGGGACTAGCGGAGCCAGTGAGCAGTTCTTC |
| 1953 | 64-TL663-TIL-CD8+_CD103− | TIL | CD8+_CD103− | TGTGCCAGCAGTTTTTCAGATACGCAGTATTTT |
| 1954 | 64-TL663-TIL-CD8+_CD103− | TIL | CD8+_CD103− | TGCGCCAGCAGCTTGGGTGGCACTGAAGCTTTCTTT |
| 1955 | 64-TL663-TIL-CD8+_CD103− | TIL | CD8+_CD103− | TGTGCCAGTAGTATAGGAGGTACGGACGAGCAGTACTTC |
| 1956 | 64-TL663-TIL-CD8+_CD103− | TIL | CD8+_CD103− | TGTGCCAGCAGCCCCCAACGAACCACGAACACTGAAGCUTCUT |
| 1957 | 64-TL663-TIL-CD8+_CD103− | TIL | CD8+_CD103− | TGTGCCAGCAGCTTAGTCCCAGGGAGCTCCTACAATGAGCAGTTCTTC |
| 1958 | 64-TL663-TIL-CD8+_CD103− | TIL | CD8+_CD103− | TGTGCCAGCAGTCCGTCGGGACAGGACTACGAGCAGTACTTC |
| 1959 | 64-TL663-TIL-CD8+_CD103− | TIL | CD8+_CD103− | TGCAGTGCTAGAGATCGGTCTAGCGGGAGGAUGGTGCAGATACGCAGTATTTT |
| 1960 | 64-TL663-TIL-CD8+_CD103− | TIL | CD8+_CD103− | TGCAGCGTTCCCTTGTTCCGGACTAGCGGGGGGGCCGATTGGGAGCAGTTC |
| | | | | TTC |
| 1961 | 64-TL663-TIL-CD8+_CD103− | TIL | CD8+_CD103− | TGCAGTGCTAGTCCAGATACCTACGAGCAGTACTTC |
| 1962 | 64-TL663-TIL-CD8+_CD103− | TIL | CD8+_CD103− | TGTGCCAGCAGCTATACCGTGGCCCACACCGGGGAGCTGTTTTTT |
| 1963 | 64-TL663-TIL-CD8+_CD103− | TIL | CD8+_CD103− | TGTGCCAGCAGCGCCACAGGGGACCTGAACACTGAAGCTTTCTTT |
| 1964 | 64-TL663-TIL-CD8+_CD103− | TIL | CD8+_CD103− | TGTGCCAGCAGCTTAAGCATAGGCACAGGCACCTTTGACGAGCAGTACTTC |
| 1965 | 64-TL663-TIL-CD8+_CD103− | TIL | CD8+_CD103− | TGCGCCAGCAGCTTCACAGGGGCCTACGAGCAGTACTTC |
| 1966 | 64-TL663-TIL-CD8+_CD103− | TIL | CD8+_CD103− | TGTGCCAGCAGCCCAGGGACTCTTGGGCAGTTCTTC |
| 1967 | 64-TL663-TIL-CD8+_CD103− | TIL | CD8+_CD103− | TGTGCCAGCAGGTTAAAGGGGACAGGGATGAACACTGAAGCTTTCTTT |
| 1968 | 64-TL663-TIL-CD8+_CD103− | TIL | CD8+_CD103− | TGTGCCAGCAGTTTCGGACTGGGGATTTACGAGCAGTACTTC |
| 1969 | 64-TL663-TIL-CD8+_CD103− | TIL | CD8+_CD103− | TGCGCCAGCAGCTTGTCTTGGCAGTACGAGCAGTACTTC |
| 1970 | 64-TL663-TIL-CD8+_CD103− | TIL | CD8+_CD103− | TGTGCCACAACCCCCGGGACAGCTTCTGAAAAACTGTTTTTT |
| 1971 | 64-TL663-TIL-CD8+_CD103− | TIL | CD8+_CD103− | TGTGCCAGCAGTTTCCGCGACTCCTTGGGCGAGCAGTACTTC |
| 1972 | 64-TL663-TIL-CD8+_CD103− | TIL | CD8+_CD103− | TGTGCCAGCAGCTTATGGTCGCTAGCGGCCAAAGAGCCCCAGTACTTC |
| 1973 | 64-TL663-TIL-CD8+_CD103− | TIL | CD8+_CD103− | TGTGCCAGCAGCTTTGGACAGGGGTTCTACGAGCAGTACTTC |
| 1974 | 64-TL663-TIL-CD8+_CD103− | TIL | CD8+_CD103− | TGTGCCAGCAGCTTATATGGCGGCTACGAGCAGTACTTC |
| 1975 | 64-TL663-TIL-CD8+_CD103− | TIL | CD8+_CD103− | TGTGCCAGCAGCTCCGACAGAAGGGAAAAACTGTTTTTT |
| 1976 | 64-TL663-TIL-CD8+_CD103− | TIL | CD8+_CD103− | TGTGCCAGCAGTGGACGCAGGGTCGGCACAGATACGCAGTATTTT |
| 1977 | 64-TL663-TIL-CD8+_CD103− | TIL | CD8+_CD103− | TGTGCCAGCAGTTTCGACATTTCTAACTATGGCTACACCTTC |
| 1978 | 64-TL663-TIL-CD8+_CD103− | TIL | CD8+_CD103− | TGTGCCACCAGCGCCGGGACAGGAGACTACGAGCAGTACTTC |
| 1979 | 64-TL663-TIL-CD8+_CD103− | TIL | CD8+_CD103− | TGTGCCAGCCTCGTAGGTGGCAATCAGCCCCAGCATTTT |
| 1980 | 64-TL663-TIL-CD8+_CD103− | TIL | CD8+_CD103− | TGCAGTGCTAGAGGTGCAACCGGGGAGCTGTTTTTT |
| 1981 | 64-TL663-TIL-CD8+_CD103− | TIL | CD8+_CD103− | TGTGCCAGCAGCTTACCCCCGACAGGGCCGGATTACGAGCAGTACTTC |
| 1982 | 64-TL663-TIL-CD8+_CD103− | TIL | CD8+_CD103− | TGTGCCAGCAGCTTGGAGGAAAACATTCAGTACTTC |
| 1983 | 64-TL663-TIL-CD8+_CD103− | TIL | CD8+_CD103− | TGTGCCAGCAGCCTGCTGTTAGGGAGCAATCAGCCCCAGCATTTT |
| 1984 | 64-TL663-TIL-CD8+_CD103− | TIL | CD8+_CD103− | TGTGCCAGCAGCCCCTTGACAGGGGGCGCGAACACTGAAGCTTTCTTT |
| 1985 | 64-TL663-TIL-CD8+_CD103− | TIL | CD8+_CD103− | TGTGCCAGCAGTTACTCGTCGACAGGGGAGTACTTC |
| 1986 | 64-TL663-TIL-CD8+_CD103− | TIL | CD8+_CD103− | TGTGCCAGCAGTTTGGGCCCGGCGGGGGAGCAGTACTTC |
| 1987 | 64-TL663-TIL-CD8+_CD103− | TIL | CD8+_CD103− | TGTGCCAGCAGCTTCAGTGCGGGAGGGCCATACGATGAGCAGTTCTTC |
| 1988 | 64-TL663-TIL-CD8+_CD103− | TIL | CD8+_CD103− | TGTGCCAGCAGTTACTCCCTAGCGGCCAGCTCCTACAATGAGCAGTTCTTC |
| 1989 | 64-TL663-TIL-CD8+_CD103− | TIL | CD8+_CD103− | TGTGCCAGCAGTTTAGTCCCAGGGAACACTGAAGCTTTCTTT |
| 1990 | 64-TL663-TIL-CD8+_CD103− | TIL | CD8+_CD103− | TGTGCCAGCAGCTTTGGCACAAGGACTAGCGGGAGGTACTCG |
| | | | | ATCCAGTTCTTC |
| 1991 | 64-TL663-TIL-CD8+_CD103− | TIL | CD8+_CD103− | TGTGCCTGGAGTTGGGGAAGCGGGGGTGAGCAGTACTTC |
| 1992 | 64-TL663-TIL-CD8+_CD103− | TIL | CD8+_CD103− | TGTGCCAGCAGCCCGAGGCTCGGTGAGCAGTTCTTC |
| 1993 | 64-TL663-TIL-CD8+_CD103− | TIL | CD8+_CD103− | TGTGCCACCAGCAGAGGGGTAGGGGGAGCAACTAATGAAAAACTGTTTTTT |
| 1994 | 64-TL663-TIL-CD8+_CD103− | TIL | CD8+_CD103− | TGTGCCAGCAGTTTAGGGGGACAGAACTATGGCTACACCTTC |
| 1995 | 64-TL663-TIL-CD8+_CD103− | TIL | CD8+_CD103− | TGTGCCAGCAGCCCAACAAGCCCAGGGGCCACTGAAGCTTTCTTT |
| 1996 | 64-TL663-TIL-CD8+_CD103− | TIL | CD8+_CD103− | TGCAGTACGAACACTGAAGCTTTCTTT |
| 1997 | 64-TL663-TIL-CD8+_CD103− | TIL | CD8+_CD103− | TGTGCCAGCAGTTCACAGGGCAATAAGATCGAGCAGTACTTC |
| 1998 | 64-TL663-TIL-CD8+_CD103− | TIL | CD8+_CD103− | TGCGCCAGCAGCTTGTACTCTGAGGACGGGAACTACGAGCAGTACTTC |
| 1999 | 64-TL663-TIL-CD8+_CD103− | TIL | CD8+_CD103− | TGTGCCTGGAGTACTTGGGACAGGGAGGCCACCGGGGAGCTGTTTTTT |
| 2000 | 64-TL663-TIL-CD8+_CD103− | TIL | CD8+_CD103− | TGTGCCAGCACAGCGGGCTCGAACACCGGGGAGCTGTTTTTT |
| 2001 | 64-TL663-TIL-CD8+_CD103− | TIL | CD8+_CD103− | TGTGCCAGCAGTTCCACCGAGATTCAGCCCCAGCATTTT |
| 2002 | 64-TL663-TIL-CD8+_CD103− | TIL | CD8+_CD103− | TGTGCCAGCAGCTTCGGGGCGGGGGGGACAGAGACCCAGTACTTC |
| 2003 | 64-TL663-TIL-CD8+_CD103− | TIL | CD8+_CD103− | TGCAGCGTTGGGGGATCAGCGGACCGCTCCTACAATGAGCAGTTCTTC |
| 2004 | 64-TL663-TIL-CD8+_CD103− | TIL | CD8+_CD103− | TGTGCCACCAGTGACCCCACTAGCGGGAGCTACGAGCAGTACTTC |
| 2005 | 64-TL663-TIL-CD8+_CD103− | TIL | CD8+_CD103− | TGTGCCAGCAGTTTATCTCGCACAGATACGCAGTATTTT |
| 2006 | 64-TL663-TIL-CD8+_CD103− | TIL | CD8+_CD103− | TGTGCCAGCAGGCTAGCCGGTAACGAGCAGTACTTC |
| 2007 | 64-TL663-TIL-CD8+_CD103− | TIL | CD8+_CD103− | TGCAGCGTTGCTGGCACAGATACGCAGTATTTT |
| 2008 | 64-TL663-TIL-CD8+_CD103− | TIL | CD8+_CD103− | TGTGCCAGCAGTTTAGGGACAGGGGCAGGCCTAGAGGACTACACCTTC |
| 2009 | 64-TL663-TIL-CD8+_CD103− | TIL | CD8+_CD103− | TGTGCCAGCAGTCCTAACAGGGGTGGGTATTCACCCCTCCACTTT |
| 2010 | 64-TL663-TIL-CD8+_CD103− | TIL | CD8+_CD103− | TGTGCCAGCAGTTTATTGGGGTGGAAGACGAACACCGGGGAGCTGTTTTTT |
| 2011 | 64-TL663-TIL-CD8+_CD103− | TIL | CD8+_CD103− | TGTGCCAGCAGGTTGGGACAGGGGGACTATGGCTACACCTTC |
| 2012 | 64-TL663-TIL-CD8+_CD103− | TIL | CD8+_CD103− | TGTGCCAGCAGCCCACCTGGGGCCAACGTCCTGACTTTC |
| 2013 | 64-TL663-TIL-CD8+_CD103− | TIL | CD8+_CD103− | TGTGCCAGCAGTGTACAGGGGGGTGGCTATGGCTACACCTTC |
| 2014 | 64-TL663-TIL-CD8+_CD103− | TIL | CD8+_CD103− | TGTGCCAGCAGCCAAGTGCAGGGGTTCGCCGGGGAGCTGTTTTTT |
| 2015 | 64-TL663-TIL-CD8+_CD103− | TIL | CD8+_CD103− | TGTGCCAGCAGCCCCAGGGGCGGGACGGCCCGATACAATGAGCAGTTCTTC |
| 2016 | 64-TL663-TIL-CD8+_CD103− | TIL | CD8+_CD103− | TGTGCCAGCAGCTTAGTGGACAGTTACAATGAGCAGTTCTTC |
| 2017 | 64-TL663-TIL-CD8+_CD103− | TIL | CD8+_CD103− | TGTGCCAGCAGTGCAACAGGGGGATATAGTCAGCCCCAGCATTTT |
| 2018 | 64-TL663-TIL-CD8+_CD103− | TIL | CD8+_CD103− | TGTGCCAGCAGCTTGGACAGCTCTGGAAACACCATATATTTT |
| 2019 | 64-TL663-TIL-CD8+_CD103− | TIL | CD8+_CD103− | TGTGCCAGCAGTTTATTTCTCTTCGAGCAGTACTTC |
| 2020 | 64-TL663-TIL-CD8+_CD103− | TIL | CD8+_CD103− | TGTGCCAGCAGTCACCAAAGTTCTGGTCAGCCCCAGCATTTT |
| 2021 | 64-TL663-TIL-CD8+_CD103− | TIL | CD8+_CD103− | TGCAGTGTACGTTCCCTAACCTCCTACGAGCAGTACTTC |
| 2022 | 64-TL663-TIL-CD8+_CD103− | TIL | CD8+_CD103− | TGTGCCTGGAGTGTACAGCTGCTTCCTAAGGGTGTTGAGCAGTTCTTC |
| 2023 | 64-TL663-TIL-CD8+_CD103− | TIL | CD8+_CD103− | TGTGCCGGGGGGGACAGGGCGGACTCTGGGGCCAACGTCCTGACTTTC |
| 2024 | 64-TL663-TIL-CD8+_CD103− | TIL | CD8+_CD103− | TGTGCCAGTAGTATATTGAAAGGCTACGAGCAGTACTTC |
| 2025 | 64-TL663-TIL-CD8+_CD103− | TIL | CD8+_CD103− | TGTGCCAGCAGCTTTACAGACAGTAGTGAGCAGTTCTTC |
| 2026 | 64-TL663-TIL-CD8+_CD103− | TIL | CD8+_CD103− | TGTGCCAGCAGCTTAGAGCTTACGGGCACAGATACGCAGTATTTT |
| 2027 | 64-TL663-TIL-CD8+_CD103− | TIL | CD8+_CD103− | TGTGCCAGCAGCTTAGGTCACGAATCCTACGAGCAGTACTTC |
| 2028 | 64-TL663-TIL-CD8+_CD103− | TIL | CD8+_CD103− | TGTGCCAGCAGCACTTCGGACAGGGGGCTTGCCGGGGAGCTGTTTTTT |
| 2029 | 64-TL663-TIL-CD8+_CD103− | TIL | CD8+_CD103− | TGTGCCAGCAGACTTATAGAGGGTTCCGAGCAGTACTTC |
| 2030 | 64-TL663-TIL-CD8+_CD103− | TIL | CD8+_CD103− | TGTGCCTGGCAAGACGGTCGAACTGAAGCTTTCTTT |
| 2031 | 64-TL663-TIL-CD8+_CD103− | TIL | CD8+_CD103− | TGTGCCAGCAGCCACGGATTCTCTGGAAACACCATATATTTT |
| 2032 | 64-TL663-TIL-CD8+_CD103− | TIL | CD8+_CD103− | TGCAGCGTCAGCAAGAACACTGAAGCTTTCTTT |
| 2033 | 64-TL663-TIL-CD8+_CD103− | TIL | CD8+_CD103− | TGTGCCACCAGTGATTTGGACGCTAGCACAAACCACAATGAGCAGTTCTTC |
| 2034 | 64-TL663-TIL-CD8+_CD103− | TIL | CD8+_CD103− | TGTGCCAGCAGCTTAGCGCCGGCTAGCGGGGGGGGCGCGGATGAGCAGTTC |
| | | | | TTC |
| 2035 | 64-TL663-TIL-CD8+_CD103− | TIL | CD8+_CD103− | TGTGCCAGCAGCCCMCGGCGCCTCTGGGGCCAACGTCCTGACTTTC |
| 2036 | 64-TL663-TIL-CD8+_CD103− | TIL | CD8+_CD103− | TGTGCCAGCAGCCCGACAGCCGGGTATGGCTACACCTTC |
| 2037 | 64-TL663-TIL-CD8+_CD103− | TIL | CD8+_CD103− | TGTGCCAGCAGCTTGATAGCTACAGATACGCAGTATTTT |
| 2038 | 64-TL663-TIL-CD8+_CD103− | TIL | CD8+_CD103− | TGTGCCAGCAGCTTAGGACAGGGCTACGAGCAGTACTTC |
| 2039 | 64-TL663-TIL-CD8+_CD103− | TIL | CD8+_CD103− | TGTGCCAGCAGTTTATCGGTGCTCTCCTACAATGAGCAGTTCTTC |
| 2040 | 64-TL663-TIL-CD8+_CD103− | TIL | CD8+_CD103− | TGTGCCAGCAGCCTACATCAGGGACCTTCCTACGAGCAGTACTTC |
| 2041 | 64-TL663-TIL-CD8+_CD103− | TIL | CD8+_CD103− | TGTGCCAGCAGTTACGACGGACAGAACACTGAAGCTTTCTTT |
| 2042 | 64-TL663-TIL-CD8+_CD103− | TIL | CD8+_CD103− | TGTGCCAGCAGTTACTCGAGGTCAATTAACACCGGGGAGCTGTTTTTT |
| 2043 | 64-TL663-TIL-CD8+_CD103− | TIL | CD8+_CD103− | TGTGCCAGCAGCTTAGATCCGGGCCAAGAGACCCAGTACTTC |
| 2044 | 64-TL663-TIL-CD8+_CD103− | TIL | CD8+_CD103− | TGTGCCATTAGACAGGGGGATGAGCAGTTCTTC |
| 2045 | 64-TL663-TIL-CD8+_CD103− | TIL | CD8+_CD103− | TGTGCCACCAGTGACATCCGGGACAGGGGCCACGAGCAGTACTTC |
| 2046 | 64-TL663-TIL-CD8+_CD103− | TIL | CD8+_CD103− | TGTGCCACCCTAGGGCGGGAGGGGAGCAATGAGCAGTTCTTC |
| 2047 | 64-TL663-TIL-CD8+_CD103− | TIL | CD8+_CD103− | TGCGCCAGCAGCTCCCCTTTTCGAGCGGGAAGCTCCTACGAGCAGTACTTC |
| 2048 | 64-TL663-TIL-CD8+_CD103− | TIL | CD8+_CD103− | TGTTCCAGCAGCTCCGGGCCAAACTACGAGCAGTACTTC |
| 2049 | 64-TL663-TIL-CD8+_CD103− | TIL | CD8+_CD103− | TGTGCCAGCAGCCCGGGACTAGCTTTACTCACAGATACGCAGTATTTT |
| 2050 | 64-TL663-TIL-CD8+_CD103− | TIL | CD8+_CD103− | TGTGCCAGCAGCTTGGGGGGCCCCGGGGAGCTGTTTTTT |
| 2051 | 64-TL663-TIL-CD8+_CD103− | TIL | CD8+_CD103− | TGTGCCAGCAGCTCTAAGTGGACCTATGGCTACACCTTC |
| 2052 | 64-TL663-TIL-CD8+_CD103− | TIL | CD8+_CD103− | TGTGCCAGCAGCCAATTTTTCTGGCAGGGGGCTTTTTGTTCGAGCACT |
| | | | | GAAGCTTTCTTT |
| 2053 | 64-TL663-TIL-CD8+_CD103− | TIL | CD8+_CD103− | TGTGCCACCAGCAGAGTTGCTGGGGGAGACACAGATACGCAGTATTTT |
| 2054 | 64-TL663-TIL-CD8+_CD103− | TIL | CD8+_CD103− | TGCGCCAGCAGCTTGGGGGGCACTGAAGCTTTCTTT |
| 2055 | 64-TL663-TIL-CD8+_CD103− | TIL | CD8+_CD103− | TGTGCCAGCAGTTACTCGACAGGGGCGAAAAACACTGAAGCTTTCTTT |
| 2056 | 64-TL663-TIL-CD8+_CD103− | TIL | CD8+_CD103− | TGCAGTGCCACGCCGATGTTAGCGGCCCAAGGGAGCTCCTACAAT |
| | | | | GAGCAGTTCTTC |
| 2057 | 64-TL663-TIL-CD8+_CD103− | TIL | CD8+_CD103− | TGTGCCAGCAGCTCCCTTAACAGGGGTCTCTATAATTCACCCCTCCACTTT |
| 2058 | 64-TL663-TIL-CD8+_CD103− | TIL | CD8+_CD103− | TGTGCCACCAGCAATCGACCAGGGACAGCCGAAGAGCAGTTCTTC |
| 2059 | 64-TL663-TIL-CD8+_CD103− | TIL | CD8+_CD103− | TGTGCCAGCAGCTTAATCCACACAGATACGCAGTATTTT |
| 2060 | 64-TL663-TIL-CD8+_CD103− | TIL | CD8+_CD103− | TGTGCCAGCAGCTCGGGGGCGGCCGGGGATTCACCCCTCCACTTT |
| 2061 | 64-TL663-TIL-CD8+_CD103− | TIL | CD8+_CD103− | TGTGCCAGCAGCCAAAGTCCAGGGGGCATTCAGTACTTC |
| 2062 | 64-TL663-TIL-CD8+_CD103− | TIL | CD8+_CD103− | TGTGCCAGCAGTCCCGGGACCGATGAGCAGTTCTTC |
| 2063 | 64-TL663-TIL-CD8+_CD103− | TIL | CD8+_CD103− | TGCAGCGTTGACCACGGGACTAGCCCTCACAATGAGCAGTTCTTC |
| 2064 | 64-TL663-TIL-CD8+_CD103− | TIL | CD8+_CD103− | TGTGCCAGCAGTTTGGAGGCCGGGACTAGGTACGAGCAGTACTTC |
| 2065 | 64-TL663-TIL-CD8+_CD103− | TIL | CD8+_CD103− | TGTGCCAGCAGCGCCCGCGGCGCCAACGTCCTGACTTTC |
| 2066 | 64-TL663-TIL-CD8+_CD103− | TIL | CD8+_CD103− | TGTGCCAGCGAGAGACCGAACACCGGGGAGCTGTTTTTT |
| 2067 | 64-TL663-TIL-CD8+_CD103− | TIL | CD8+_CD103− | TGCGCCAGCAGCCTTCCAACAGCCGGCGCCAACGTCCTGACTTTC |
| 2068 | 64-TL663-TIL-CD8+_CD103− | TIL | CD8+_CD103− | TGTGCCAGCAGCCACACTAGCGGGGCGAACACCGGGGAGCTGTTTTTT |
| 2069 | 64-TL663-TIL-CD8+_CD103− | TIL | CD8+_CD103− | TGTGCCAGCGCCCTGGGACAGGCGGGGAACACTGAAGCTTTCTTT |
| 2070 | 64-TL663-TIL-CD8+_CD103− | TIL | CD8+_CD103− | TGCGCCAGCACCCCCCAGGCGCCATCCTACGAGCAGTACTTC |
| 2071 | 64-TL663-TIL-CD8+_CD103− | TIL | CD8+_CD103− | TGTGCCAGTAGGCTAGCGGGAGACAATGAGCAGTTCTTC |
| 2072 | 64-TL663-TIL-CD8+_CD103− | TIL | CD8+_CD103− | TGTGCCAGCAGCTTGGGTACCCTACCCTCCTACGAGCAGTACTTC |
| 2073 | 64-TL663-TIL-CD8+_CD103− | TIL | CD8+_CD103− | TGCGCCAGCAGCTTGGAGGGACAGGCGAGCTCCTACGAGCAGTACTTC |
| 2074 | 64-TL663-TIL-CD8+_CD103− | TIL | CD8+_CD103− | TGTGCCAGCAGTTACCTAAAGACAGGGGAGGGCTATGGCTACACCTTC |
| 2075 | 64-TL663-TIL-CD8+_CD103− | TIL | CD8+_CD103− | TGTGCCAGCAGATCGGACCAAGAGACCCAGTACTTC |
| 2076 | 64-TL663-TIL-CD8+_CD103− | TIL | CD8+_CD103− | TGTGCCAGCAGCTTGTTTAGCGGGGATGAGCAGTTCTTC |
| 2077 | 64-TL663-TIL-CD8+_CD103− | TIL | CD8+_CD103− | TGTGCCAGCAGCCCGGGACTGAACACAGATACGCAGTATTTT |
| 2078 | 64-TL663-TIL-CD8+_CD103− | TIL | CD8+_CD103− | TGTGCCAGCAGCCCCTCGGACAACCCAAACTACGAGCAGTACTTC |
| 2079 | 64-TL663-TIL-CD8+_CD103− | TIL | CD8+_CD103− | TGCGCCAGCAGTCACCGAACTAAGGACAGGGACCTTAAGGATGAG |
| | | | | CAGTTCTTC |
| 2080 | 64-TL663-TIL-CD8+_CD103− | TIL | CD8+_CD103− | TGTGCCAGCGGCTGGACAGGGCCTGAGACCCAGTACTTC |
| 2081 | 90-TL101-TIL-CD8+_CD103+ | TIL | CD8+_CD103+ | TGCGCCAGCAGCTGGGGATTCCGGCGGGACTATGGCTACACCTTC |
| 2082 | 90-TL101-TIL-CD8+_CD103+ | TIL | CD8+_CD103+ | TGTGCCATCAGTGAGTGCGGGAGCTTCACAGCGTGCCCAGATACG |
| | | | | CAGTATTTT |
| 2083 | 90-TL101-TIL-CD8+_CD103+ | TIL | CD8+_CD103+ | TGTGCCAGCAGACCCGGGGGAAGTTCGACTACTAGCACAGATACG |
| | | | | CAGTATTTT |
| 2084 | 90-TL101-TIL-CD8+_CD103+ | TIL | CD8+_CD103+ | TGTGCCAGCAGCCAGCAGGGGACCTTATGGACAGATACGCAGTATTTT |
| 2085 | 90-TL101-TIL-CD8+_CD103+ | TIL | CD8+_CD103+ | TGTGCCAGCACCCTAGGACTGCAAGAGACCCAGTACTTC |
| 2086 | 90-TL101-TIL-CD8+_CD103+ | TIL | CD8+_CD103+ | TGTGCCAGCAGCCAGGGGGGGGACGGCCCCTACAATGAGCAGTTCTTC |
| 2087 | 90-TL101-TIL-CD8+_CD103+ | TIL | CD8+_CD103+ | TGTGCCAGCAGCGCTGGGCCAGGGACGAACACTGAAGCTTTCTTT |
| 2088 | 90-TL101-TIL-CD8+_CD103+ | TIL | CD8+_CD103+ | TGTGCCAGCAGTTTACTAAGGGCCTACGAGCAGTACTTC |
| 2089 | 90-TL101-TIL-CD8+_CD103+ | TIL | CD8+_CD103+ | TGTGCCAGCAGTTACTCGGCAGGAGCCTCCTACGAGCAGTACTTC |
| 2090 | 90-TL101-TIL-CD8+_CD103+ | TIL | CD8+_CD103+ | TGTGCCAGCAGCGTTGGTATGAACACTGAAGCTTTCTTT |
| 2091 | 90-TL101-TIL-CD8+_CD103+ | TIL | CD8+_CD103+ | TGTGCCAGCAGTTTAGAGACAGGGCCGTACTTT |
| 2092 | 90-TL101-TIL-CD8+_CD103+ | TIL | CD8+_CD103+ | TGTGCCAGCAGTTCCCAATATTTTTACACTGAAGCTTTCTTT |
| 2093 | 90-TL101-TIL-CD8+_CD103+ | TIL | CD8+_CD103+ | TGTGCCAGCAGCCCTTCGGGTGAGCAGTTCTTC |
| 2094 | 90-TL101-TIL-CD8+_CD103+ | TIL | CD8+_CD103+ | TGTGCCAGCAGCTCCCGACTAGCGGGAGCTATAGATACGCAGTATTTT |
| 2095 | 90-TL101-TIL-CD8+_CD103+ | TIL | CD8+_CD103+ | TGTGCCAGCAGCTGGGGAACGGGGTACGAGCAGTACTTC |
| 2096 | 90-TL101-TIL-CD8+_CD103+ | TIL | CD8+_CD103+ | TGTGCCATCAGTGAGTCGACAGGGTTAAATACGCAGTATTTT |
| 2097 | 90-TL101-TIL-CD8+_CD103+ | TIL | CD8+_CD103+ | TGTGCCGTACTGGGGACTAGCAACGATGAGCAGTTCTTC |
| 2098 | 90-TL101-TIL-CD8+_CD103+ | TIL | CD8+_CD103+ | TGTGCCAGCAGCTCGCGAGTAGGGAGTAATTCACCCCTCCACTTT |
| 2099 | 90-TL101-TIL-CD8+_CD103+ | TIL | CD8+_CD103+ | TGTGCCAGCAGTACGGGACAGGGGGCGGATGGCTACACCTTC |
| 2100 | 90-TL101-TIL-CD8+_CD103+ | TIL | CD8+_CD103+ | TGTGCCAGCAGCCCATTGTCTAGTAGCCACAATGAGCAGTTCTTC |
| 2101 | 90-TL101-TIL-CD8+_CD103+ | TIL | CD8+_CD103+ | TGTGCCAGCAGTGCACAGACAGGGTCTTACTATGGCTACACCTTC |
| 2102 | 90-TL101-TIL-CD8+_CD103+ | TIL | CD8+_CD103+ | TGTGCCAGCAGTTTTATACAGGGACTCGATGGCTACACCTTC |
| 2103 | 90-TL101-TIL-CD8+_CD103+ | TIL | CD8+_CD103+ | TGTGCCAGCAGCTTGGGACAAGCCTACGAGCAGTACTTC |
| 2104 | 90-TL101-TIL-CD8+_CD103+ | TIL | CD8+_CD103+ | TGTGCCAGCAGCTTCCGGGACATAAGACAGTATTTT |
| 2105 | 90-TL101-TIL-CD8+_CD103+ | TIL | CD8+_CD103+ | TGCAGCGTTGATCCAGGGTATTACAATGAGCAGTTCTTC |
| 2106 | 90-TL101-TIL-CD8+_CD103+ | TIL | CD8+_CD103+ | TGTGCCAGCAGTTTCAGGACTAGCTCCTACAATGAGCAGTTCTTC |
| 2107 | 90-TL101-TIL-CD8+_CD103+ | TIL | CD8+_CD103+ | TGCGCCAGCAGTCCTCATATCCAGAGCTCCTACGAGCAGTACTTC |
| 2108 | 90-TL101-TIL-CD8+_CD103+ | TIL | CD8+_CD103+ | TGCAGTGCTAGAGATATGGAGGGCAAGGTCGATGAGCAGTTCTTC |
| 2109 | 90-TL101-TIL-CD8+_CD103+ | TIL | CD8+_CD103+ | TGTGCCAGCAGCGTAGGGGGGACTCCGTTCAATGAGCAGTTCTTC |
| 2110 | 90-TL101-TIL-CD8+_CD103+ | TIL | CD8+_CD103+ | TGTGCCACCCAAAGTACTAGCGGGATATCCACCGGGGAGCTGTTTTTT |
| 2111 | 90-TL101-TIL-CD8+_CD103+ | TIL | CD8+_CD103+ | TGCGCCAGCAGCCAACCAGATACGCAGTATTTT |
| 2112 | 90-TL101-TIL-CD8+_CD103+ | TIL | CD8+_CD103+ | TGTGCCAGCAGTTTCGGAGGGAACACTGAAGCTTTCTTT |
| 2113 | 90-TL101-TIL-CD8+_CD103+ | TIL | CD8+_CD103+ | TGTGCCAGCAGGGCCGAGGAAATCTATAGCAATCAGCCCCAGCATTTT |
| 2114 | 90-TL101-TIL-CD8+_CD103+ | TIL | CD8+_CD103+ | TGTGCCAGCGGGCCGGGACTAGCGGGAGGGCTTTACGAGCAGTACTTC |
| 2115 | 90-TL101-TIL-CD8+_CD103+ | TIL | CD8+_CD103+ | TGTGCCAGCAGCCAAGGAGTGACGGAGACGGAGACCCAGTACTTC |
| 2116 | 90-TL101-TIL-CD8+_CD103+ | TIL | CD8+_CD103+ | TGTGCCAGCAGTATCAGGACAGGGCCAAATAGCAATCAGCCCCAGCATTTT |
| 2117 | 90-TL101-TIL-CD8+_CD103+ | TIL | CD8+_CD103+ | TGTGCCAGCAGTGAAACGAAGATCAGTAGCACAGATACGCAGTATTTT |
| 2118 | 90-TL101-TIL-CD8+_CD103+ | TIL | CD8+_CD103+ | TGTGCCAGCAGCCTAACCGTGGACAGGGGGCCTCTCTTC |
| 2119 | 90-TL101-TIL-CD8+_CD103+ | TIL | CD8+_CD103+ | TGCAGTGCTAGGGGAACAGGGCTCGACTCCTACGAGCAGTACTTC |
| 2120 | 90-TL101-TIL-CD8+_CD103+ | TIL | CD8+_CD103+ | TGTGCCAGCACCTCACTCCCGGAGAGGTTGGAGACCCAGTACTTC |
| 2121 | 90-TL101-TIL-CD8+_CD103+ | TIL | CD8+_CD103+ | TGTGCCAGCAGTTTCAGCGGGATGGGTTCCTACGAGCAGTACTTC |
| 2122 | 90-TL101-TIL-CD8+_CD103+ | TIL | CD8+_CD103+ | TGTGCCAGCAGTGGGTTAAGAGGGATGAGCAATCAGCCCCAGCATTTT |
| 2123 | 90-TL101-TIL-CD8+_CD103+ | TIL | CD8+_CD103+ | TGCAGCGTTGAAGAGAGGGCGTGGGGAATGAGTGAGACCCAGTACTTC |
| 2124 | 90-TL101-TIL-CD8+_CD103+ | TIL | CD8+_CD103+ | TGTGCCAGCAGTGTTACAGAAAACACCGGGGAGCTGTTTTTT |
| 2125 | 90-TL101-TIL-CD8+_CD103+ | TIL | CD8+_CD103+ | TGTGCCAGCAGTTACGGGACAAGTCTCAATGAGCAGTTCTTC |
| 2126 | 90-TL101-TIL-CD8+_CD103+ | TIL | CD8+_CD103+ | TGTGCCAGCAGTTGGATCAACCCGGGACTAGCCTCGAACTAC |
| | | | | GAGCAGTACTTC |
| 2127 | 90-TL101-TIL-CD8+_CD103+ | TIL | CD8+_CD103+ | TGTGCCAGCATTGTGGGGAGGTCGCCTGCCGGTGAGCAGTTCTTC |
| 2128 | 91-TL101-TIL-CD8+_CD103− | TIL | CD8+_CD103− | TGTGCCAGCAGCTTGGGACAAGCCTACGAGCAGTACTTC |
| 2129 | 91-TL101-TIL-CD8+_CD103− | TIL | CD8+_CD103− | TGTGCCAGCAGCTGGGGAACGGGGTACGAGCAGTACTTC |
| 2130 | 91-TL101-TIL-CD8+_CD103− | TIL | CD8+_CD103− | TGTGCCAGCAGCGACCTTCTAGACATTGAGGCCGGGGAGCTGTTTTTT |
| 2131 | 91-TL101-TIL-CD8+_CD103− | TIL | CD8+_CD103− | TGCAGCGTGCAAGGACTAGCGGAAGGCTCCTACAATGAGCAGTTCTTC |
| 2132 | 91-TL101-TIL-CD8+_CD103− | TIL | CD8+_CD103− | TGTGCCAGCAGGTCGGGACTAGCGGGAGGCTGGGAGCAGTTCTTC |
| 2133 | 91-TL101-TIL-CD8+_CD103− | TIL | CD8+_CD103− | TGTGCCAGCAGCCTAACCGTGGACAGGGGGCCTCTCTTC |
| 2134 | 91-TL101-TIL-CD8+_CD103− | TIL | CD8+_CD103− | TGTGCCAGCAGTTACACTAGCGGGGACAATGAGCAGTTCTTC |
| 2135 | 91-TL101-TIL-CD8+_CD103− | TIL | CD8+_CD103− | TGCAGCTTTGCCCGGACTAGCGGCGGCGGTGAGCAGTTCTTC |
| 2136 | 91-TL101-TIL-CD8+_CD103− | TIL | CD8+_CD103− | TGTGCCAGCAGTGCCCGACCTACCTCGCAGGGGCCCCAGCATTTT |
| 2137 | 91-TL101-TIL-CD8+_CD103− | TIL | CD8+_CD103− | TGTGCCAGCAGTTTAAGCCAGGGGACCCAGCCCCAGCATTTT |
| 2138 | 91-TL101-TIL-CD8+_CD103− | TIL | CD8+_CD103− | TGCGCCAGCAGCCAAGGGTTAGCGGTTAGCTCCTACGAGCAGTACTTC |
| 2139 | 91-TL101-TIL-CD8+_CD103− | TIL | CD8+_CD103− | TGTGCCAGCAGTTCCGGACCGAGCACTGAGCAGTTCTTC |
| 2140 | 91-TL101-TIL-CD8+_CD103− | TIL | CD8+_CD103− | TGTGCCAGCAGCTTACTAGCGGGAGTCGACACCGGGGAGCTGTTTTTT |
| 2141 | 91-TL101-TIL-CD8+_CD103− | TIL | CD8+_CD103− | TGTGCCAGCACCCTAGGACTGCAAGAGACCCAGTACTTC |
| 2142 | 91-TL101-TIL-CD8+_CD103− | TIL | CD8+_CD103− | TGCGCCAGCAGCCAAGTTGGGACGAGCGGCAGCCCCTACGAGCAGTACTTC |
| 2143 | 91-TL101-TIL-CD8+_CD103− | TIL | CD8+_CD103− | TGTGCCAGTTTAGCGGGAGGAAACACCGGGGAGCTGTTTTTT |
| 2144 | 91-TL101-TIL-CD8+_CD103− | TIL | CD8+_CD103− | TGCAGTGCCGGCCGTTCTAGGGAGCTGTTTTTT |
| 2145 | 91-TL101-TIL-CD8+_CD103− | TIL | CD8+_CD103− | TGTGCCAGCAGCCAAGAGAACGAGCAGTACTTC |
| 2146 | 91-TL101-TIL-CD8+_CD103− | TIL | CD8+_CD103− | TGTGCCAGCAGGAAGGGACTAGCGGGAGTAAGGACAGATACGCAGTATTTT |
| 2147 | 91-TL101-TIL-CD8+_CD103− | TIL | CD8+_CD103− | TGCGCCAGCAGTTACAGCGGGGGGAACACCGGGGAGCTGTTTTTT |
| 2148 | 91-TL101-TIL-CD8+_CD103− | TIL | CD8+_CD103− | TGTGCCAGCAGGGATCTAGGGAATGAGCAGTTCTTC |
| 2149 | 91-TL101-TIL-CD8+_CD103− | TIL | CD8+_CD103− | TGTGCCAGCAGCTTATATCCGGACCTTGAAGCTTTCTTT |
| 2150 | 91-TL101-TIL-CD8+_CD103− | TIL | CD8+_CD103− | TGTGCCATCCGTCAGTGGGCTGATAGCAATCAGCCCCAGCATTTT |
| 2151 | 91-TL101-TIL-CD8+_CD103− | TIL | CD8+_CD103− | TGTGCCAGCAGCTCGGGACTAGCGACGGATGAGCAGTTCTTC |
| 2152 | 91-TL101-TIL-CD8+_CD103− | TIL | CD8+_CD103− | TGTGCCAGCAGCTCTGGGACAGGGGAGGGCTATGAGCAGTTCTTC |
| 2153 | 91-TL101-TIL-CD8+_CD103− | TIL | CD8+_CD103− | TGTGCCAGCAAAGGACTAGCGGGAGCTGGGACCCAGTACTTC |
| 2154 | 91-TL101-TIL-CD8+_CD103− | TIL | CD8+_CD103− | TGTGCCAGCAGCCCTTCGTCAGGGGGGAGGGCCAGGGATACGCAGTATTTT |
| 2155 | 91-TL101-TIL-CD8+_CD103− | TIL | CD8+_CD103− | TGCGCCAGCAGCCAGGGACTAGCGGGAGGGCCGAATGAGCAGTTCTTC |
| 2156 | 91-TL101-TIL-CD8+_CD103− | TIL | CD8+_CD103− | TGTGCCAGCAGCCGACAGGGCATTTATTCACCCCTCCACTTT |
| 2157 | 91-TL101-TIL-CD8+_CD103− | TIL | CD8+_CD103− | TGTGCCACCAGCCCCACCCGGGCCCAGTATGAGCAGTTCTTC |
| 2158 | 91-TL101-TIL-CD8+_CD103− | TIL | CD8+_CD103− | TGCAGTGGGGGGACAGGGCCCACTGAAGCTTTCTTT |
| 2159 | 91-TL101-TIL-CD8+_CD103− | TIL | CD8+_CD103− | TGTGCCACCAGCAGAGAGAGAGGACGGTCTTCCTACGAGCAGTACTTC |
| 2160 | 91-TL101-TIL-CD8+_CD103− | TIL | CD8+_CD103− | TGTGCCAGCAGCTTGGGACTGGGCCTTCTTACGCAGTATTTT |
| 2161 | 91-TL101-TIL-CD8+_CD103− | TIL | CD8+_CD103− | TGCGCCAGCAGCCCGGGACAGGGTGAAGGGTACGAGCAGTACTTC |
| 2162 | 91-TL101-TIL-CD8+_CD103− | TIL | CD8+_CD103− | TGTGCCAGCAGTTTACTAAGGGCCTACGAGCAGTACTTC |
| 2163 | 91-TL101-TIL-CD8+_CD103− | TIL | CD8+_CD103− | TGTGCCTGGGGGGACAGTAACACCGGGGAGCTGTTTTTT |
| 2164 | 91-TL101-TIL-CD8+_CD103− | TIL | CD8+_CD103− | TGTGCCAGCAGGGGCCCATTGGGACCGAATCAGCCCCAGCATTTT |
| 2165 | 91-TL101-TIL-CD8+_CD103− | TIL | CD8+_CD103− | TGCGCCAGCAGCCCTGGGGGCAGCACAGATACGCAGTATTTT |
| 2166 | 91-TL101-TIL-CD8+_CD103− | TIL | CD8+_CD103− | TGTGCAAGTCGCCCATGGTTGGGACAGGGAAACACCGGGGAGCTGTTTTTT |
| 2167 | 91-TL101-TIL-CD8+_CD103− | TIL | CD8+_CD103− | TGTGCCAGCAGCGCTGCAGGGGAGCGGAGCTACGAGCAGTACTTC |
| 2168 | 91-TL101-TIL-CD8+_CD103− | TIL | CD8+_CD103− | TGTGCCAGCAGTTACGACAGGGGCCTTTATGGCTACACCTTC |
| 2169 | 91-TL101-TIL-CD8+_CD103− | TIL | CD8+_CD103− | TGTGCCAGCAGGTCAACAGGGGGCGGTCAGCCCCAGCATTTT |
| 2170 | 91-TL101-TIL-CD8+_CD103− | TIL | CD8+_CD103− | TGTGCCAGCAGACAGGACAGGGGGTTTTCCTACGAGCAGTACTTC |
| 2171 | 91-TL101-TIL-CD8+_CD103− | TIL | CD8+_CD103− | TGTGCCAGCAGCCCATTGTCTAGTAGCCACAATGAGCAGTTCTTC |
| 2172 | 91-TL101-TIL-CD8+_CD103− | TIL | CD8+_CD103− | TGCAGTGCTAGGGAACAGGGAGGGGGCTACACCTTC |
| 2173 | 91-TL101-TIL-CD8+_CD103− | TIL | CD8+_CD103− | TGTGCCAGCAGCTGGTCTGGCGCTCGCACAGATACGCAGTATTTT |
| 2174 | 91-TL101-TIL-CD8+_CD103− | TIL | CD8+_CD103− | TGTGCCACCAGTGATTTAAGCGGGTGGAACACCGGGGAGCTGTTTTTT |
| 2175 | 91-TL101-TIL-CD8+_CD103− | TIL | CD8+_CD103− | TGTGCCAGCTCCTCCACGGGAGAGACCCAGTACTTC |
| 2176 | 91-TL101-TIL-CD8+_CD103− | TIL | CD8+_CD103− | TGTGCCAGCAGTCCTAGCGCGTTCGAGCAGTACTTC |
| 2177 | 91-TL101-TIL-CD8+_CD103− | TIL | CD8+_CD103− | TGTGCCACCTCCCGGCAGGGACAGGGCACAGATACGCAGTATTTT |
| 2178 | 91-TL101-TIL-CD8+_CD103− | TIL | CD8+_CD103− | TGTGCCAGCAGCTTGGTGACCCGGGCCACTGAAGCTTTCTTT |
| 2179 | 91-TL101-TIL-CD8+_CD103− | TIL | CD8+_CD103− | TGCAGCGTTAAGCCCGACAGGGGGCGGTACGAGCAGTACTTC |
| 2180 | 91-TL101-TIL-CD8+_CD103− | TIL | CD8+_CD103− | TGTGCCAGCAGCCAAGTGTCCCTAGCGGGAGTTCAAGAGACCCAGTACTTC |
| 2181 | 91-TL101-TIL-CD8+_CD103− | TIL | CD8+_CD103− | TGTGCCAGCAGCCGCCCAAATTCCGGGACTAGCTTCGTGGAGACCCAGTAC |
| | | | | TTC |
| 2182 | 91-TL101-TIL-CD8+_CD103− | TIL | CD8+_CD103− | TGTGCCAGCAGCTTAACGGAGCTGGACTACGAGCAGTACTTC |
| 2183 | 91-TL101-TIL-CD8+_CD103− | TIL | CD8+_CD103− | TGTGCCATCAGTGAGAGCAGGGAGGCGAGTGAAAAACTGTTTTTT |
| 2184 | 91-TL101-TIL-CD8+_CD103− | TIL | CD8+_CD103− | TGTGCCAGCAGCTTCCCGTTCGGTGAAGCTTTCTTT |
| 2185 | 91-TL101-TIL-CD8+_CD103− | TIL | CD8+_CD103− | TGTGCCAGCAGCCAAAGCCCAGGCGGGACCCAGTACTTC |
| 2186 | 91-TL101-TIL-CD8+_CD103− | TIL | CD8+_CD103− | TGTGCCAGCAGCCAAGATCCAAGACTAGCGGGACCCGCC |
| | | | | GCAGATACGCAGTATTTT |
| 2187 | 91-TL101-TIL-CD8+_CD103− | TIL | CD8+_CD103− | TGTGCCAGCAGTTGGAGAAGAGGGGCGGAGACCCAGTACTTC |
| 2188 | 91-TL101-TIL-CD8+_CD103− | TIL | CD8+_CD103− | TGTGCCAGCAGTTTCGGCAACGCGAGGAGCAATCAGCCCCAGCATTTT |
| 2189 | 91-TL101-TIL-CD8+_CD103− | TIL | CD8+_CD103− | TGTGCCAGCAGCCAGACTAGCGGGGAGCACGCTACGCAGTATTTT |
| 2190 | 91-TL101-TIL-CD8+_CD103− | TIL | CD8+_CD103− | TGTGCCTGGAACACAGCAAACACTGAAGCTTTCTTT |
| 2191 | 91-TL101-TIL-CD8+_CD103− | TIL | CD8+_CD103− | TGCGCCAGCAGCTTGGCCCCCAGTGTGAGGTTTCAAGAGACCCAGTACTTC |
| 2192 | 91-TL101-TIL-CD8+_CD103− | TIL | CD8+_CD103− | TGTGCCAGCAGCTACCCAGGGGCCGGGACTGAAGCTTTCTTT |
| 2193 | 91-TL101-TIL-CD8+_CD103− | TIL | CD8+_CD103− | TGTGCCAGCAGCCCAGCTGGGACTAGGGTCATTCAGTACTTC |
| 2194 | 91-TL101-TIL-CD8+_CD103− | TIL | CD8+_CD103− | TGTGCCAGCTCACCAAACTCTAAGTACGAGCAGTACTTC |
| 2195 | 91-TL101-TIL-CD8+_CD103− | TIL | CD8+_CD103− | TGTGCCAGCAGCCAAGGACCGGGACAGGGGGGGACTTTT |
| 2196 | 91-TL101-TIL-CD8+_CD103− | TIL | CD8+_CD103− | TGTGCCAGCAGCGGGCTATCCTACGAGCAGTACTTC |
| 2197 | 91-TL101-TIL-CD8+_CD103− | TIL | CD8+_CD103− | TGCAGTGCCCATCAGACAGTCTCATACACAGATACGCAGTATTTT |
| 2198 | 91-TL101-TIL-CD8+_CD103− | TIL | CD8+_CD103− | TGCAGTGCCGAGACGGACACAGATACGCAGTATTTT |
| 2199 | 91-TL101-TIL-CD8+_CD103− | TIL | CD8+_CD103− | TGCAGTGCTCGCCGGGACCCCGGGGAGCTGTTTTTT |
| 2200 | 91-TL101-TIL-CD8+_CD103− | TIL | CD8+_CD103− | TGCGCCAGCAGCTTTTTCCAGGGGGGCGCTGAAGCTTTCTTT |
| 2201 | 91-TL101-TIL-CD8+_CD103− | TIL | CD8+_CD103− | TGTGCCAGCAGATTATTAAGCGGCAGGGGGCGGGATGGCTACACCTTC |
| 2202 | 91-TL101-TIL-CD8+_CD103− | TIL | CD8+_CD103− | TGTGCCAGCAGCTTAGTCGTGGCGGGCGGGCTGAACAATGAGCAGTTCTTC |
| 2203 | 91-TL101-TIL-CD8+_CD103− | TIL | CD8+_CD103− | TGTGCCAGCAGCCCTACCTACGGGACTAGCGTCAGACTCACAGATACGCAG |
| | | | | TATTTT |
| 2204 | 91-TL101-TIL-CD8+_CD103− | TIL | CD8+_CD103− | TGTGCCAGCAGCGGGACAGGGGTAAGGGTTTATAGCAATCAGCCCCAGCAT |
| | | | | TTT |
| 2205 | 91-TL101-TIL-CD8+_CD103− | TIL | CD8+_CD103− | TGTGCCAGCAGCCCGAACAGGGGCTCTAACTATGGCTACACCTTC |
| 2206 | 91-TL101-TIL-CD8+_CD103− | TIL | CD8+_CD103− | TGTGCCAGCAGCCTCAGGGGGGGGCAAGTAGGGAACACTGAAGCTTTCTTT |
| 2207 | 91-TL101-TIL-CD8+_CD103− | TIL | CD8+_CD103− | TGTGCCAGCAGTTTTATCACAGTGCTCGCGGATCTAGCCAAAAACATTCAG |
| | | | | TACTTC |
| 2208 | 91-TL101-TIL-CD8+_CD103− | TIL | CD8+_CD103− | TGTGCCAGCTTTCGAGACGGACGCATCGGAAACACTGAAGCTTTCTTT |
| 2209 | 91-TL101-TIL-CD8+_CD103− | TIL | CD8+_CD103− | TGCGCCAGCAGCCAAGAATCTTCAGGGATGAGGGCCGGGGAGCTGTTTTTT |
| 2210 | 91-TL101-TIL-CD8+_CD103− | TIL | CD8+_CD103− | TGTGCCAGCAGCGTATCCAGACAGGGCAGCTATGGCTACACCTTC |
| 2211 | 91-TL101-TIL-CD8+_CD103− | TIL | CD8+_CD103− | TGTGCCATCAGTGATACTACCCCCTCAGATACGCAGTATTTT |
| 2212 | 91-TL101-TIL-CD8+_CD103− | TIL | CD8+_CD103− | TGTGCCAGCAGCTTCGAGCGGCAAGAGACCCAGTACTTC |
| 2213 | 91-TL101-TIL-CD8+_CD103− | TIL | CD8+_CD103− | TGTGCCTGGAGTGTACCGGGATTCGGACGGAACAATCAGCCCCAGCATTTT |
| 2214 | 91-TL101-TIL-CD8+_CD103− | TIL | CD8+_CD103− | TGCGCCAGCAGCTTGGACCTAGCGGGAGGGCTGAAAGGGGTCTTC |
| 2215 | 91-TL101-TIL-CD8+_CD103− | TIL | CD8+_CD103− | TGCAGTGCTGGGGGAGGCGGATCGTGGCCCTCTCAAGAGACCCAGTACTTC |
| 2216 | 91-TL101-TIL-CD8+_CD103− | TIL | CD8+_CD103− | TGTGCCACCAGCAGAATGGGGACTAGCGGGAGAGGGGATACGCAGTATTTT |
| 2217 | 91-TL101-TIL-CD8+_CD103− | TIL | CD8+_CD103− | TGTGCCAGCAGTTTAGCGCGGCAAAGTGGCTACACCTTC |
| 2218 | 91-TL101-TIL-CD8+_CD103− | TIL | CD8+_CD103− | TGTGCCAGCAGCTACAGGGGGGCTAGCACAGATACGCAGTATTTT |
| 2219 | 91-TL101-TIL-CD8+_CD103− | TIL | CD8+_CD103− | TGTGCCAGCAGTTTATGGGCGGGAGGAGCAGATACGCAGTATTTT |
| 2220 | 91-TL101-TIL-CD8+_CD103− | TIL | CD8+_CD103− | TGTGCCAGCAGCTTAGGGACTAGCGGGCTTTCGAATGAGCAGTTCTTC |
| 2221 | 91-TL101-TIL-CD8+_CD103− | TIL | CD8+_CD103− | TGTGCCAGCAGACCATATGGGACACCTAATAGCAATCAGCCCCAGCATTTT |
| 2222 | 91-TL101-TIL-CD8+_CD103− | TIL | CD8+_CD103− | TGTGCCAGCACTCAAGGGGCAGCGAACACTGAAGCTTTCTTT |
| 2223 | 91-TL101-TIL-CD8+_CD103− | TIL | CD8+_CD103− | TGTGCCAGCAGTTACTCGAGCCGTACTGAAGCTTTCTTT |
| 2224 | 91-TL101-TIL-CD8+_CD103− | TIL | CD8+_CD103− | TGCAGTGCTAGAGATTCAGGGGACGCTGGGGCCAACGTCCTGACTTTC |
| 2225 | 91-TL101-TIL-CD8+_CD103− | TIL | CD8+_CD103− | TGTGCCAGCAGTTTAGTCGGAGTATACAATGAGCAGTTCTTC |
| 2226 | 91-TL101-TIL-CD8+_CD103− | TIL | CD8+_CD103− | TGCAGTGCTATGCGGGGTTCGGGGTTCGGAGAGACCCAGTACTTC |
| 2227 | 91-TL101-TIL-CD8+_CD103− | TIL | CD8+_CD103− | TGTGCCAGCAGTTTACCCAGGGGGCGAGACGAGCAGTACTTC |
| 2228 | 91-TL101-TIL-CD8+_CD103− | TIL | CD8+_CD103− | TGTGCCAGCAGCTTAACGGGGAGAATTTACAATGAGCAGTTCTTC |
| 2229 | 91-TL101-TIL-CD8+_CD103− | TIL | CD8+_CD103− | TGTGCCAGCAGTTTAACCGGAGCTGGCTACACCTTC |
| 2230 | 91-TL101-TIL-CD8+_CD103− | TIL | CD8+_CD103− | TGCGCCAGCAGCTTGGGTCCTAGCGGGCCGCTCGGAGAGACCCAGTACTTC |
| 2231 | 91-TL101-TIL-CD8+_CD103− | TIL | CD8+_CD103− | TGTGCCAGCAGCTTACCCGTATCCGAGCAGTACTTC |
| 2232 | 91-TL101-TIL-CD8+_CD103− | TIL | CD8+_CD103− | TGTGCCAGCAGGGGCGGGAGTAAGGCAGATACGCAGTATTTT |
| 2233 | 91-TL101-TIL-CD8+_CD103− | TIL | CD8+_CD103− | TGTGCCAGCACCACCGACAGCAATAATGAAAAACTGTTTTTT |
| 2234 | 91-TL101-TIL-CD8+_CD103− | TIL | CD8+_CD103− | TGCAGTGCTAGAAGACCCGGGACTAGCGGAACCTACGAGCAGTACTTC |
| 2235 | 91-TL101-TIL-CD8+_CD103− | TIL | CD8+_CD103− | TGTGCCAGCAGTGAAGGGACTAGCGGGAGAGCCGGGGAGCTGTTTTTT |
| 2236 | 91-TL101-TIL-CD8+_CD103− | TIL | CD8+_CD103− | TGTGCCAGCAGTCCCCGGGAACCAGCCTCTAACTATGGCTACACCTTC |
| 2237 | 91-TL101-TIL-CD8+_CD103− | TIL | CD8+_CD103− | TGCAGTGCTAGCGACACCGGACTAGCCGGGGAGACCCAGTACTTC |
| 2238 | 91-TL101-TIL-CD8+_CD103− | TIL | CD8+_CD103− | TGTGCCAGCAGTGAAGCTGTGGTGGCAGGCTATGGCTACACCTTC |
| 2239 | 91-TL101-TIL-CD8+_CD103− | TIL | CD8+_CD103− | TGTGCCAGCTCAACGTTTTACACTGAAGCTTTCTTT |
| 2240 | 91-TL101-TIL-CD8+_CD103− | TIL | CD8+_CD103− | TGTGCCAGCAGCCCGGCCCTTGCGGGAAATGAGCAGTTCTTC |
| 2241 | 91-TL101-TIL-CD8+_CD103− | TIL | CD8+_CD103− | TGTGCCAGCAGTTCAGACTCCGGAGTCCCGTACGAGCAGTACTTC |
| 2242 | 91-TL101-TIL-CD8+_CD103− | TIL | CD8+_CD103− | TGTGCCAGCAGTTTTCGGGGCTCGTCCTACGAGCAGTACTTC |
| 2243 | 91-TL101-TIL-CD8+_CD103− | TIL | CD8+_CD103− | TGTGCCAGCAGCGTAGCCGGGGAAGCAAGCTACGAGCAGTACTTC |
| 2244 | 91-TL101-TIL-CD8+_CD103− | TIL | CD8+_CD103− | TGCAGCGTCCGGGTGGGGGGAGGCAATCAGCCCCAGCATTTT |
| 2245 | 91-TL101-TIL-CD8+_CD103− | TIL | CD8+_CD103− | TGTGCCAGCAGCCCGAGTGCTAGCGGGAGAGCGGATACGCAGTATTTT |
| 2246 | 91-TL101-TIL-CD8+_CD103− | TIL | CD8+_CD103− | TGTGCCAGCTCACCACCCCGGGGGCAGGGTGACACTGAAGCTTTCTTT |
| 2247 | 91-TL101-TIL-CD8+_CD103− | TIL | CD8+_CD103− | TGTGCCTGGACCCGGACTAGCGGATCCCAGTTCTTC |
| 2248 | 91-TL101-TIL-CD8+_CD103− | TIL | CD8+_CD103− | TGCGCCAGCAGCTTGGCGGGGGAGGGCCTCTCCAATGAGCAGTTCTTC |
| 2249 | 91-TL101-TIL-CD8+_CD103− | TIL | CD8+_CD103− | TGTGCCAGCAGGGTCCCAAGCGTGGAGACCCAGTACTTC |
| 2250 | 91-TL101-TIL-CD8+_CD103− | TIL | CD8+_CD103− | TGTGCCAGCAGCCCGGGTGGGCGGACGGGAGTTATGAACACTGAAG |
| | | | | CTTTCTTT |
| 2251 | 91-TL101-TIL-CD8+_CD103− | TIL | CD8+_CD103− | TGTGCCAGCAGCTTGGAACTAGCGGGAGGCGAGCAGTACTTC |
| 2252 | 91-TL101-TIL-CD8+_CD103− | TIL | CD8+_CD103− | TGTGCCATCAGGAGGGCCGACGATGAGCAGTTCTTC |
| 2253 | 91-TL101-TIL-CD8+_CD103− | TIL | CD8+_CD103− | TGTGCCACCAGCAGAGGGGACATCTCTGGGGCCAACGTCCTGACTTTC |
| 2254 | 91-TL101-TIL-CD8+_CD103− | TIL | CD8+_CD103− | TGTGCCAGCAAGGGGGGCTCCAATCAGCCCCAGCATTTT |
| 2255 | 91-TL101-TIL-CD8+_CD103− | TIL | CD8+_CD103− | TGCGCCAGCAGCCACGGACCGCCCTTGCTCGAGCAGTACTTC |
| 2256 | 91-TL101-TIL-CD8+_CD103− | TIL | CD8+_CD103− | TGTGCCAGCAGTGAAATCTCGACCGGGACAGGGACCAATGAGCAGTTCTTC |
| 2257 | 91-TL101-TIL-CD8+_CD103− | TIL | CD8+_CD103− | TGTGCCAGCAGCTCCCGAGGACGGCCGGAAAATGAGCAGTTCTTC |
| 2258 | 91-TL101-TIL-CD8+_CD103− | TIL | CD8+_CD103− | TGTGCAAGTCGCTTCGGGACAGGGATTATCCAAGAGACCCAGTACTTC |
| 2259 | 91-TL101-TIL-CD8+_CD103− | TIL | CD8+_CD103− | TGTGCCAGCTCACCTCTAAGGGAGGGGCAGTACTTC |
| 2260 | 91-TL101-TIL-CD8+_CD103− | TIL | CD8+_CD103− | TGCAGTGCTAGAGACCCGATGACTAGCGGGAGTTTCTATGAGCAGTTCTTC |
| 2261 | 91-TL101-TIL-CD8+_CD103− | TIL | CD8+_CD103− | TGTGCCAGCAGTTTGTCAGGAACGCGTGGCTACACCTTC |
| 2262 | 91-TL101-TIL-CD8+_CD103− | TIL | CD8+_CD103− | TGTGCCAGCGGGCGGGATTTAGGAAGGTCCAACGAGCAGTACTTC |
| 2263 | 91-TL101-TIL-CD8+_CD103− | TIL | CD8+_CD103− | TGTGCCAGCAGCAGGACGGAACGGAACACTGAAGCTTTCTTT |
| 2264 | 91-TL101-TIL-CD8+_CD103− | TIL | CD8+_CD103− | TGTGCCAGCAGCTTAGTCGGAGGGACTAACTATGGCTACACCTTC |
| 2265 | 91-TL101-TIL-CD8+_CD103− | TIL | CD8+_CD103− | TGTGCCATCAGTCGCAGGGAATTCAACAATGAGCAGTTCTTC |
| 2266 | 91-TL101-TIL-CD8+_CD103− | TIL | CD8+_CD103− | TGTGCCACCAGCAGAGATCGAGGTTCAAGCGGTGAGCAGTTCTTC |
| 2267 | 91-TL101-TIL-CD8+_CD103− | TIL | CD8+_CD103− | TGTGCCAGCAGCTCAGGGGGCTTCATGTTCTATGGCTACACCTTC |
| 2268 | 91-TL101-TIL-CD8+_CD103− | TIL | CD8+_CD103− | TGCGCCAGCAGAACAGCTCTAGCGGGGGGAGGTGAGCAGTTCTTC |
| 2269 | 91-TL101-TIL-CD8+_CD103− | TIL | CD8+_CD103− | TGTGCCAGCAGCTCCGAGAGACAGGCATCTTTCTACGAGCAGTACTTC |
| 2270 | 91-TL101-TIL-CD8+_CD103− | TIL | CD8+_CD103− | TGTGCCAGCAGTCCAGACAGACCAAGTAGGGTCTTC |
| 2271 | 91-TL101-TIL-CD8+_CD103− | TIL | CD8+_CD103− | TGCAGTGCTAGGCTGACAGGGGAGGACACTGAAGCTTTCTTT |
| 2272 | 91-TL101-TIL-CD8+_CD103− | TIL | CD8+_CD103− | TGTGCCAGCAGCCTCAGCTACGAGCAGTACTTC |
| 2273 | 91-TL101-TIL-CD8+_CD103− | TIL | CD8+_CD103− | TGTGCCAGCAGCTTAACGTCTTCGGCGTTGGGGGCCGGGGAGCTGTTTTTT |
| 2274 | 91-TL101-TIL-CD8+_CD103− | TIL | CD8+_CD103− | TGTGCCAGCAGTTCTCGTGAGGGGTCGGGCACTGAAGCTTTCTTT |
| 2275 | 91-TL101-TIL-CD8+_CD103− | TIL | CD8+_CD103− | TGCGCCAGCAGCTTGGAGGGGTCTCAGCCCCAGCATTTT |
| 2276 | 91-TL101-TIL-CD8+_CD103− | TIL | CD8+_CD103− | TGTGCTCTTGGGGAAGCGGGCTCCTACACCGGGGAGCTGTTTTTT |
| 2277 | 91-TL101-TIL-CD8+_CD103− | TIL | CD8+_CD103− | TGTGCCAGCAGCTTCAGGGGGCGACGCCAAGAGACCCAGTACTTC |
| 2278 | 91-TL101-TIL-CD8+_CD103− | TIL | CD8+_CD103− | TGTGCCAGCAGTCCCGGGTCAGGCCAACCCATTCAGTACTTC |
| 2279 | 91-TL101-TIL-CD8+_CD103− | TIL | CD8+_CD103− | TGTGCCAGCTCACCACATCCGGGACAACCCTACGAGCAGTACTTC |
| 2280 | 91-TL101-TIL-CD8+_CD103− | TIL | CD8+_CD103− | TGCGCCAGCAGCTTGGAAAGGGACTACGAGCAGTACTTC |
| 2281 | 91-TL101-TIL-CD8+_CD103− | TIL | CD8+_CD103− | TGTGCCAGCAGCCCTACAGGGATCGATCAGCCCCAGCATTTT |
| 2282 | 91-TL101-TIL-CD8+_CD103− | TIL | CD8+_CD103− | TGTGCCAGCAGCCGAAGGGTGGAAAACACCGGGGAGCTGTTTTTT |
| 2283 | 91-TL101-TIL-CD8+_CD103− | TIL | CD8+_CD103− | TGTGCCAGCAGCTTGGGAGTAGATACGCAGTATTTT |
| 2284 | 91-TL101-TIL-CD8+_CD103− | TIL | CD8+_CD103− | TGTGCCAGCAGCCGGACAGGGGCCACTGAAGCTTTCTTT |
| 2285 | 91-TL101-TIL-CD8+_CD103− | TIL | CD8+_CD103− | TGCAGTGCTAGAGGCACGACAGAAGAAGCTTTCTTT |
| 2286 | 91-TL101-TIL-CD8+_CD103− | TIL | CD8+_CD103− | TGTGCCAGCAGCCCCCGACGAGTGAGCCCCTACGAGCAGTACTTC |
| 2287 | 91-TL101-TIL-CD8+_CD103− | TIL | CD8+_CD103− | TGTGCCAGCAGCTTATTAGCGGGAGGGCCTTCCGGTGAGCAGTTCTTC |
| 2288 | 91-TL101-TIL-CD8+_CD103− | TIL | CD8+_CD103− | TGTGCCAGCCGCCCTCCGGGCGCGGGAGTTATTGTGGGGCAAGAG |
| | | | | ACCCAGTACTTC |
| 2289 | 91-TL101-TIL-CD8+_CD103− | TIL | CD8+_CD103− | TGTGCCAGCGGCCAGACAACAGGGCGGACTGAAGCTTTCTTT |
| 2290 | 91-TL101-TIL-CD8+_CD103− | TIL | CD8+_CD103− | TGTGCCAGCAGAGGGACCTGGAACACCGGGGAGCTGTTTTTT |
| 2291 | 91-TL101-TIL-CD8+_CD103− | TIL | CD8+_CD103− | TGTGCCAGCAGCCGTCTAGGGTACAATGAGCAGTTCTTC |
| 2292 | 91-TL101-TIL-CD8+_CD103− | TIL | CD8+_CD103− | TGTGCCAGCAGCCCCCGTCCGGGGGAGAGGAACACTGAAGCTTTCTTT |
| 2293 | 91-TL101-TIL-CD8+_CD103− | TIL | CD8+_CD103− | TGTGCCACCCGATCGGGACAGGGGAACACCGGGGAGCTGTTTTTT |
| 2294 | 91-TL101-TIL-CD8+_CD103− | TIL | CD8+_CD103− | TGTGCCAGCTCACCAGGAAGCGCATGGGACTCCTCTAATGAAAAA |
| | | | | CTGTTTTTT |
| 2295 | 91-TL101-TIL-CD8+_CD103− | TIL | CD8+_CD103− | TGCGCCAGCAGTAGAGCGCGCAATGAGCAGTTCTTC |
| 2296 | 91-TL101-TIL-CD8+_CD103− | TIL | CD8+_CD103− | TGTGCCAGCAGCCAAGCATTCAGGGGCGAGCAGTACTTC |
| 2297 | 91-TL101-TIL-CD8+_CD103− | TIL | CD8+_CD103− | TGCAGTGCTACCCCCCAAGACCACGTGGAGCAGTTCTTC |
| 2298 | 91-TL101-TIL-CD8+_CD103− | TIL | CD8+_CD103− | TGCAGTGCTAGGGGACTAGCCTACGAGCAGTACTTC |
| 2299 | 91-TL101-TIL-CD8+_CD103− | TIL | CD8+_CD103− | TGTGCCTGCTCGCGATTGGGGAGGGCCTATTACAATGAGCAGTTCTTC |
| 2300 | 91-TL101-TIL-CD8+_CD103− | TIL | CD8+_CD103− | TGTGCCTGGAGTGGGAGACTAGCGGGAGAACCACTTATCTTC |
| 2301 | 91-TL101-TIL-CD8+_CD103− | TIL | CD8+_CD103− | TGTGCCAGCAGCTGGGGGGGGTGGGAAAAACTGTTTTTT |
| 2302 | 91-TL101-TIL-CD8+_CD103− | TIL | CD8+_CD103− | TGTGCCAGTAGTGAACAGCGGGACAGGGGCAATGAGCAGTTCTTC |
| 2303 | 91-TL101-TIL-CD8+_CD103− | TIL | CD8+_CD103− | TGCAGCGTTGAAGGGGGCACCCCGACTGGGTATGGCTACACCTTC |
| 2304 | 91-TL101-TIL-CD8+_CD103− | TIL | CD8+_CD103− | TGTGCCAGCAGCTTAGGATCGACAGTTTACTACGAGCAGTACTTC |
| 2305 | 91-TL101-TIL-CD8+_CD103− | TIL | CD8+_CD103− | TGTGCCATCAGTGGTAAAGCGGGAGTTAATCCCGGGGAGCTGTTTTTT |
| 2306 | 91-TL101-TIL-CD8+_CD103− | TIL | CD8+_CD103− | TGTGCCAGCAGCGACGGGACAGGCGGGGGGAATGAAAAACTGTTTTTT |
| 2307 | 91-TL101-TIL-CD8+_CD103− | TIL | CD8+_CD103− | TGTGCCAGCAGCTCCCTACAGGGGGTTTTGGGAGAGACCCAGTACTTC |
| 2308 | 91-TL101-TIL-CD8+_CD103− | TIL | CD8+_CD103− | TGTGCCAGCAGCCAAGAGAGGGGCTACGAGCAGTACTTC |
| 2309 | 91-TL101-TIL-CD8+_CD103− | TIL | CD8+_CD103− | TGTGCCAGCAGTACGACAGGGGGTGCTAACTATGGCTACACCTTC |
| 2310 | 91-TL101-TIL-CD8+_CD103− | TIL | CD8+_CD103− | TGTGCCAGCAGTGTACAGGGGGCTGGTGGCTACACCTTC |
| 2311 | 91-TL101-TIL-CD8+_CD103− | TIL | CD8+_CD103− | TGTGCCAGCAGTTTAGGGGGGGACTCTGGAAACACCATATATTTT |
| 2312 | 91-TL101-TIL-CD8+_CD103− | TIL | CD8+_CD103− | TGTGCCAGTAGAGCGCCCACGAACACTGAAGCTTTCTTT |
| 2313 | 91-TL101-TIL-CD8+_CD103− | TIL | CD8+_CD103− | TGTGCCAGCAGCTTTATTTCAGGGGAAAGGGGTGAGCAGTTCTTC |
| 2314 | 91-TL101-TIL-CD8+_CD103− | TIL | CD8+_CD103− | TGTGCCTGGAGTGAGGGCGGGAGTCTCTACGAGCAGTACTTC |
| 2315 | 91-TL101-TIL-CD8+_CD103− | TIL | CD8+_CD103− | TGTGCCAGCAGTTACGGGACCGACTACGAGCAGTACTTC |
| 2316 | 91-TL101-TIL-CD8+_CD103− | TIL | CD8+_CD103− | TGTGCCAGCTCACCGGGGGGCTACAATGAGCAGTTCTTC |
| 2317 | 91-TL101-TIL-CD8+_CD103− | TIL | CD8+_CD103− | TGTGCCAGCAGTTTGAACCCCCGGAAGGGCTCCTACAATGAGCAGTTCTTC |
| 2318 | 91-TL101-TIL-CD8+_CD103− | TIL | CD8+_CD103− | TGCGCCAGCAGTGTACGCCGTGGCAATGAGCAGTTCTTC |
| 2319 | 91-TL101-TIL-CD8+_CD103− | TIL | CD8+_CD103− | TGTGCCAGCAGCCCCCAAGATACGCAGTATTTT |
| 2320 | 91-TL101-TIL-CD8+_CD103− | TIL | CD8+_CD103− | TGTGCCAGCAGCTCCCAGACAGGGACCTACGAGCAGTACTTC |
| 2321 | 91-TL101-TIL-CD8+_CD103− | TIL | CD8+_CD103− | TGCAGTGCTAAGAGAGGCGGCTCCTACAATGAGCAGTTCTTC |
| 2322 | 91-TL101-TIL-CD8+_CD103− | TIL | CD8+_CD103− | TGTGCCAGCAGCGAGGGGACGCTTGGCACCGGGGAGCTGTTTTTT |
| 2323 | 91-TL101-TIL-CD8+_CD103− | TIL | CD8+_CD103− | TGTGCCAGCAGTTTAAGCGGGACAGGGGGAGAAAAACTGTTTTTT |
| 2324 | 91-TL101-TIL-CD8+_CD103− | TIL | CD8+_CD103− | TGCGCCAGCAGCTTGAGGTCTAGCGGCTTGATTGGTGAGCAGTTCTTC |
| 2325 | 91-TL101-TIL-CD8+_CD103− | TIL | CD8+_CD103− | TGTGCCATCAGTGAGCTAAGGGAGCCCCCCTACAATGAGCAGTTCTTC |
| 2326 | 91-TL101-TIL-CD8+_CD103− | TIL | CD8+_CD103− | TGCAGTGCTAGAGATGACAGAACGACGAGCTCCTATAATTCACCCCTC |
| | | | | CACTTT |
| 2327 | 91-TL101-TIL-CD8+_CD103− | TIL | CD8+_CD103− | TGCAGTGCTAGGGGAACAGGGCTCGACTCCTACGAGCAGTACTTC |
| 2328 | 91-TL101-TIL-CD8+_CD103− | TIL | CD8+_CD103− | TGTGCCAGCAGCTTAGGGGGCCTTTGGACTAGCCCGGGTAGCTC |
| | | | | CTACAATGAGCAGTTCTTC |
| 2329 | 91-TL101-TIL-CD8+_CD103− | TIL | CD8+_CD103− | TGTGCCAGCAGTGGTCAGGGGGGACAGACCCAGTACTTC |
| 2330 | 91-TL101-TIL-CD8+_CD103− | TIL | CD8+_CD103− | TGTGCCAGCTCACCACTCAAGGACAGGACTTACCCCTACGAGCAGTACTTC |
| 2331 | 91-TL101-TIL-CD8+_CD103− | TIL | CD8+_CD103− | TGTGCCAGCAGCCCCTGGACTAGCGGCACAGATACGCAGTATTTT |
| 2332 | 91-TL101-TIL-CD8+_CD103− | TIL | CD8+_CD103− | TGTGCCAGCAGCTCGACGGTTTACGAGCAGTACTTC |
| 2333 | 91-TL101-TIL-CD8+_CD103− | TIL | CD8+_CD103− | TGTGCCATCAGTGAGTCGGCAGGGAGCAACACTGAAGCTTTCTTT |
| 2334 | 91-TL101-TIL-CD8+_CD103− | TIL | CD8+_CD103− | TGTGCCACCAGCATCCCCGACAGGGCCCAGCAGTACTTC |
| 2335 | 91-TL101-TIL-CD8+_CD103− | TIL | CD8+_CD103− | TGTGCCACCAAGCAGGGGGCGAGGACAGATACGCAGTATTTT |
| 2336 | 91-TL101-TIL-CD8+_CD103− | TIL | CD8+_CD103− | TGTGCCAGCAGCTCTGGACAGGGTTTCGCCTACGAGCAGTACTTC |
| 2337 | 91-TL101-TIL-CD8+_CD103− | TIL | CD8+_CD103− | TGCAGCACGAGAGGGACTAGCGGTTTTTATCCCTCCCTCGCTGGG |
| | | | | GCCAACGTCCTGACTTTC |
| 2338 | 91-TL101-TIL-CD8+_CD103− | TIL | CD8+_CD103− | TGTGCCAGCAGCCGACGGACTAGCGGAACCTACAATGAGCAGTTCTTC |
| 2339 | 91-TL101-TIL-CD8+_CD103− | TIL | CD8+_CD103− | TGTGCCAGCAGTGCCCAGTACGGCGGAAATCAGCCCCAGCATTTT |
| 2340 | 91-TL101-TIL-CD8+_CD103− | TIL | CD8+_CD103− | TGTGCCAGCAGCCAAGGACTAGCGGGTTACAATGAGCAGTTCTTC |
| 2341 | 91-TL101-TIL-CD8+_CD103− | TIL | CD8+_CD103− | TGCGCCAGCAGACCGGGACTAGCGGCCTACAATGAGCAGTTCTTC |
| 2342 | 91-TL101-TIL-CD8+_CD103− | TIL | CD8+_CD103− | TGTGCCAGCTCACCCGACAGGGGAGGAAATACGCAGTATTTT |
| 2343 | 91-TL101-TIL-CD8+_CD103− | TIL | CD8+_CD103− | TGTGCCAGCAGCTTGGAGGGGGCTGGAAAACTGTTTTTT |
| 2344 | 91-TL101-TIL-CD8+_CD103− | TIL | CD8+_CD103− | TGCGCCAGCAGCTATGGGAGCTCCTACAATGAGCAGTTCTTC |
| 2345 | 91-TL101-TIL-CD8+_CD103− | TIL | CD8+_CD103− | TGTGCCAGCAGCAATCCCAGGGACTCGGCAGATACGCAGTATTTT |
| 2346 | 91-TL101-TIL-CD8+_CD103− | TIL | CD8+_CD103− | TGTGCCAGCAGTTACTCGACGGCGAGCTGGCAGTTCCAAGAGA |
| | | | | CCCAGTACTTC |
| 2347 | 91-TL101-TIL-CD8+_CD103− | TIL | CD8+_CD103− | TGTGCCAGTCGGGACAGCACCTACGAGCAGTACTTC |
| 2348 | 91-TL101-TIL-CD8+_CD103− | TIL | CD8+_CD103− | TGTGCCAGTAGGGCGGACAGGGGAGGGAATCAGCCCCAGCATTTT |
| 2349 | 91-TL101-TIL-CD8+_CD103− | TIL | CD8+_CD103− | TGTGCCAGCAGTCCCAGGGGTGTAGGGACTGAAGCTTTCTTT |
| 2350 | 91-TL101-TIL-CD8+_CD103− | TIL | CD8+_CD103− | TGTGCCAGCAGCTTATCCGGGAATAGCAATCAGCCCCAGCATTTT |
| 2351 | 91-TL101-TIL-CD8+_CD103− | TIL | CD8+_CD103− | TGTGCCGGACAGGACTCCTACAATGAGCAGTTCTTC |
| 2352 | 91-TL101-TIL-CD8+_CD103− | TIL | CD8+_CD103− | TGCGCCAGCAGCCAACCCACCGGGCGGGGGTACGAGCAGTACTTC |
| 2353 | 91-TL101-TIL-CD8+_CD103− | TIL | CD8+_CD103− | TGTGCCAGCAGCCTCACAGGGAGTGAGACCCAGTACTTC |
| 2354 | 91-TL101-TIL-CD8+_CD103− | TIL | CD8+_CD103− | TGTGCCAGCAGCCTCCGGCTAGCGGATCGTACAAATGAGCAGTTCTTC |
| 2355 | 91-TL101-TIL-CD8+_CD103− | TIL | CD8+_CD103− | TGTGCCAGCAGCCAGAGACAGGCAACGACCACAGATACGCAGTATTTT |
| 2356 | 91-TL101-TIL-CD8+_CD103− | TIL | CD8+_CD103− | TGCAGTGCTAGTCGGGCAGATACGCAGTATTTT |
| 2357 | 91-TL101-TIL-CD8+_CD103− | TIL | CD8+_CD103− | TGTGCCAGCAGACGGACTAGCGGGAGGGCCGATGAGCAGTTCTTC |
| 2358 | 91-TL101-TIL-CD8+_CD103− | TIL | CD8+_CD103− | TGTGCCAGCAGTGAAGACATGAATCAGCCCCAGCATTTT |
| 2359 | 91-TL101-TIL-CD8+_CD103− | TIL | CD8+_CD103− | TGTGCCAGCTCACTTACGACAGGGGGTAACACTGAAGCTTTCTTT |
| 2360 | 91-TL101-TIL-CD8+_CD103− | TIL | CD8+_CD103− | TGTGCCAGCAGCCCCCTCATTGGGATTACCTACAATGAGCAGTTCTTC |
| 2361 | 91-TL101-TIL-CD8+_CD103− | TIL | CD8+_CD103− | TGTGCCAGCAGCTTGGACAGGGTATGGACTGAAGCTTTCTTT |
| 2362 | 91-TL101-TIL-CD8+_CD103− | TIL | CD8+_CD103− | TGTGCCAGCAGCCAGGGGTCACTCACAGATACGCAGTATTTT |
| 2363 | 91-TL101-TIL-CD8+_CD103− | TIL | CD8+_CD103− | TGTGCCAGCAGCTTTTTCGGCCCGAACACCGGGGAGCTGTTTTTT |
| 2364 | 91-TL101-TIL-CD8+_CD103− | TIL | CD8+_CD103− | TGTGCCAGCAGTTACGCTGTCCCGGGACTAGCGGGCTCGACCTA |
| | | | | CAATGAGCAGTTCTTC |
| 2365 | 91-TL101-TIL-CD8+_CD103− | TIL | CD8+_CD103− | TGTGCCAGCAGCCCGGACAGTAATCAGCCCCAGCATTTT |
| 2366 | 91-TL101-TIL-CD8+_CD103− | TIL | CD8+_CD103− | TGTGCCAGCAGTTCCCCGGGACAGGCAGGTTCACCCCTCCACTTT |
| 2367 | 91-TL101-TIL-CD8+_CD103− | TIL | CD8+_CD103− | TGTGCCAGCAGCTTAAATGGGGATACGCAGTATTTT |
| 2368 | 91-TL101-TIL-CD8+_CD103− | TIL | CD8+_CD103− | TGTGCCAGCAGTTTATTACAGAATGTTTCACCCCTCCACTTT |
| 2369 | 91-TL101-TIL-CD8+_CD103− | TIL | CD8+_CD103− | TGTGCCAGCAGTCCTTCACCGGGACAGGGGCCCAATGAGCAGTTCTTC |
| 2370 | 91-TL101-TIL-CD8+_CD103− | TIL | CD8+_CD103− | TGTGCCATCAGTGAGTCGAGAGGGCGGGACTCTACAGATACGCAGTATTTT |
| 2371 | 91-TL101-TIL-CD8+_CD103− | TIL | CD8+_CD103− | TGTGCCAGCAGCCAGGGAGGACAGGGAGGGAACGAGCAGTACTTC |
| 2372 | 91-TL101-TIL-CD8+_CD103− | TIL | CD8+_CD103− | TGCAGCGTTGAATTGCGGGGAGCCTACGAGCAGTACTTC |
| 2373 | 91-TL101-TIL-CD8+_CD103− | TIL | CD8+_CD103− | TGTGCCAGCAGCTTGCTTCAGGGGAGGAATCAGCCCCAGCATTTT |
| 2374 | 91-TL101-TIL-CD8+_CD103− | TIL | CD8+_CD103− | TGTGCCAGTAGTAAGTCCCAGCTCAATCAGCCCCAGCATTTT |
| 2375 | 91-TL101-TIL-CD8+_CD103− | TIL | CD8+_CD103− | TGTGCCAGCAGCTTGCGGGAGGGGGTTCTCGGCAATGAGCAGTTCTTC |
| 2376 | 91-TL101-TIL-CD8+_CD103− | TIL | CD8+_CD103− | TGCAGCGTCGACCTAGTCACCGGGGAGCTGTTTTTT |
| 2377 | 91-TL101-TIL-CD8+_CD103− | TIL | CD8+_CD103− | TGTGCTAGTGGTTCCGGGTACAATGAGCAGTTCTTC |
| 2378 | 91-TL101-TIL-CD8+_CD103− | TIL | CD8+_CD103− | TGTGCCAGCAGTTCCCCCCCAGGGAAGGCCACTGAAGCTTTCTTT |
| 2379 | 91-TL101-TIL-CD8+_CD103− | TIL | CD8+_CD103− | TGCAGTGCTAGACACGGGACTAGCAGTTACGAGCAGTACTTC |
| 2380 | 91-TL101-TIL-CD8+_CD103− | TIL | CD8+_CD103− | TGCGCCAGCAGCTGGGGGGGACAGGGGTTGGACGACTATGGCTACACCTTC |
| 2381 | 91-TL101-TIL-CD8+_CD103− | TIL | CD8+_CD103− | TGCAGCGTTGAGTCCCCCAGGGGCAGAGAGACCCAGTACTTC |
| 2382 | 91-TL101-TIL-CD8+_CD103− | TIL | CD8+_CD103− | TGTGCCAGCAGCCCGGGCCTACCCGATCCGGAGACCCAGTACTTC |
| 2383 | 91-TL101-TIL-CD8+_CD103− | TIL | CD8+_CD103− | TGTGCCAGCTCACCAGGGAATCTTAACTACTCTTACTACGAGCAGTACTTC |
| 2384 | 91-TL101-TIL-CD8+_CD103− | TIL | CD8+_CD103− | TGTGCCAGCAGTTTGACCGGGACAGGGAAAGGCTACACCTTC |
| 2385 | 91-TL101-TIL-CD8+_CD103− | TIL | CD8+_CD103− | TGTGCCAGCAGCTTTGAGATGTACTACGAGCAGTACTTC |
| 2386 | 91-TL101-TIL-CD8+_CD103− | TIL | CD8+_CD103− | TGTGCCAGCAGTAGCATCGGCGCCTCGGGGTCGGATACGCAGTATTTT |
| 2387 | 91-TL101-TIL-CD8+_CD103− | TIL | CD8+_CD103− | TGTGCCAGCAGTTACTCCCGTACTAGCGGAATCCCCTCCTACA |
| | | | | CAGATACGCAGTATTTT |
| 2388 | 91-TL101-TIL-CD8+_CD103− | TIL | CD8+_CD103− | TGTGCCAGCAGCGCAGGGCCGGGAGTCGATCAGCCCCAGCATTTT |
| 2389 | 91-TL101-TIL-CD8+_CD103− | TIL | CD8+_CD103− | TGTGCCAGCCAGGGTTCCTACAATGAGCAGTTCTTC |
| 2390 | 91-TL101-TIL-CD8+_CD103− | TIL | CD8+_CD103− | TGCAGTGCTAGGGTTGTGACTAGCGGGAGTAACAATGAGCAGTTCTTC |
| 2391 | 91-TL101-TIL-CD8+_CD103− | TIL | CD8+_CD103− | TGTGCCAGCAACTCGGACGGGAGCTCCTACAATGAGCAGTTCTTC |
| 2392 | 91-TL101-TIL-CD8+_CD103− | TIL | CD8+_CD103− | TGCGCCAGCAGCTTGGACCCGGGACTGCTCACCGGGGAGCTGTTTTTT |
| 2393 | 91-TL101-TIL-CD8+_CD103− | TIL | CD8+_CD103− | TGTGCCAGCGGGTCGGCCTACGAGCAGTACTTC |
| 2394 | 91-TL101-TIL-CD8+_CD103− | TIL | CD8+_CD103− | TGTGCCAGCAGTTACGGGACAGTAACCTACGAGCAGTACTTC |
| 2395 | 91-TL101-TIL-CD8+_CD103− | TIL | CD8+_CD103− | TGTGCCAGCAGTATCTACAATGAGCAGTTCTTC |
| 2396 | 91-TL101-TIL-CD8+_CD103− | TIL | CD8+_CD103− | TGTGCCAGCAGTGAAACGAAGATCAGTAGCACAGATACGCAGTATTTT |
| 2397 | 91-TL101-TIL-CD8+_CD103− | TIL | CD8+_CD103− | TGTGCCACCAGTGATGGGAGGAGAGGGTCCTACAATGAGCAGTTCTTC |
| 2398 | 91-TL101-TIL-CD8+_CD103− | TIL | CD8+_CD103− | TGTGCCAGCAGCCCCCCCGGACAGAGCTACGAGCAGTACTTC |
| 2399 | 91-TL101-TIL-CD8+_CD103− | TIL | CD8+_CD103− | TGTGCCAGCAGCTTATCTGGGGGCACTGAAGCTTTCTTT |
| 2400 | 91-TL101-TIL-CD8+_CD103− | TIL | CD8+_CD103− | TGTGCCAGCAGTTTAGGGGGACCTGGTGCTGGCTACACCTTC |
| 2401 | 91-TL101-TIL-CD8+_CD103− | TIL | CD8+_CD103− | TGCAGCGTTCGGGGACGGGGAGATGAGCAGTTCTTC |
| 2402 | 91-TL101-TIL-CD8+_CD103− | TIL | CD8+_CD103− | TGTGCCAGTAGAGAGGGCACCGGGGAGCTGTTTTTT |
| 2403 | 91-TL101-TIL-CD8+_CD103− | TIL | CD8+_CD103− | TGCAGTGCTAGCTTACGGGACTACTACAATGAGCAGTTCTTC |
| 2404 | 91-TL101-TIL-CD8+_CD103− | TIL | CD8+_CD103− | TGTGCCAGCAGTCCCTACACGATCCACTATGGCTACACCTTC |
| 2405 | 91-TL101-TIL-CD8+_CD103− | TIL | CD8+_CD103− | TGTGCCAGCAGCTTAGTTTCCACCGGGGAGCTGTTTTTT |
| 2406 | 91-TL101-TIL-CD8+_CD103− | TIL | CD8+_CD103− | TGCGCCAGCAGCCCCACTGCAACTAATGAAAAACTGTTTTTT |
| 2407 | 91-TL101-TIL-CD8+_CD103− | TIL | CD8+_CD103− | TGTGCCAGCAGCTTAAGCGGGAGATACAATGAGCAGTTCTTC |
| 2408 | 91-TL101-TIL-CD8+_CD103− | TIL | CD8+_CD103− | TGTGCCAGCAGCTTAGAAACAGCTACGGAGACCCAGTACTTC |
| 2409 | 91-TL101-TIL-CD8+_CD103− | TIL | CD8+_CD103− | TGCAGTGCTAGAGATGAGGACTGGGGGTACAATGAGCAGTTCTTC |
| 2410 | 91-TL101-TIL-CD8+_CD103− | TIL | CD8+_CD103− | TGTGCCAGCAGCCCCTACGGGGCCTCCTACAATGAGCAGTTCTTC |
| 2411 | 91-TL101-TIL-CD8+_CD103− | TIL | CD8+_CD103− | TGTGCCAGCAGTTTATGGCAGACCCTGCCTTTCTCTGGAAAC |
| | | | | ACCATATATTTT |
| 2412 | 91-TL101-TIL-CD8+_CD103− | TIL | CD8+_CD103− | TGTGCCAGCAGTTTTCGGGGGAACACTGAAGCTTTCTTT |
| 2413 | 91-TL101-TIL-CD8+_CD103− | TIL | CD8+_CD103− | TGCGCCAGCAGCTTCGGGACTAGCGGAGGCGGGGGCAATGAGCAGTTCTTC |
| 2414 | 91-TL101-TIL-CD8+_CD103− | TIL | CD8+_CD103− | TGCAGTGCTAGAGATTTAGGGTCCAAAAACATTCAGTACTTC |
| 2415 | 91-TL101-TIL-CD8+_CD103− | TIL | CD8+_CD103− | TGTGCCAGCAGCCAAGTCCCGGGACAGGGGTACGAGCAGTACTTC |
| 2416 | 91-TL101-TIL-CD8+_CD103− | TIL | CD8+_CD103− | TGTGCCAGCAGCCCAAGCGGTCAGCTCCACTACGAGCAGTACTTC |
| 2417 | 91-TL101-TIL-CD8+_CD103− | TIL | CD8+_CD103− | TGTGCCAGCAGCCCAGGGGGTGGGGAGACCCAGTACTTC |
| 2418 | 91-TL101-TIL-CD8+_CD103− | TIL | CD8+_CD103− | TGTGCCAGCAGTTTCGGACAGATCAATCAGCCCCAGCATTTT |
| 2419 | 91-TL101-TIL-CD8+_CD103− | TIL | CD8+_CD103− | TGCAGTGCTAGAGCCCCCAACTCTGGAAACACCATATATTTT |
| 2420 | 91-TL101-TIL-CD8+_CD103− | TIL | CD8+_CD103− | TGCAGTGCGAGATCAGGCGAGAACGATTACGAGCAGTACTTC |
| 2421 | 91-TL101-TIL-CD8+_CD103− | TIL | CD8+_CD103− | TGTGCCAGCAGTTTCGAGCTTGCACGGGGCACTGAAGCTTTCTTT |
| 2422 | 91-TL101-TIL-CD8+_CD103− | TIL | CD8+_CD103− | TGTGCCAGCAGTTTAGGGTACCGGGCTAGCGCCCAAGAGACCCAGTACTTC |
| 2423 | 91-TL101-TIL-CD8+_CD103− | TIL | CD8+_CD103− | TGTGCCAGCAGCCAAGCCCTCCAAAATCAGCCCCAGCATTTT |
| 2424 | 91-TL101-TIL-CD8+_CD103− | TIL | CD8+_CD103− | TGTGCCACTAAATCTGGGGCCAACGTCCTGACTTTC |
| 2425 | 91-TL101-TIL-CD8+_CD103− | TIL | CD8+_CD103− | TGTGCCAGCAGCTTTGGGGCCGTCTATGGCTACACCTTC |
| 2426 | 91-TL101-TIL-CD8+_CD103− | TIL | CD8+_CD103− | TGTGCCAGCAGCTCGATGGGCGGGACCTTGCTGGGCACTGAAGCTTTCTTT |
| 2427 | 91-TL101-TIL-CD8+_CD103− | TIL | CD8+_CD103− | TGTGCCAGCAGCTACCCCACAGGGGTCACAGATACGCAGTATTTT |
| 2428 | 91-TL101-TIL-CD8+_CD103− | TIL | CD8+_CD103− | TGCAGTGCTAGACCGGGAGGGCCGATCAATGAGCAGTTCTTC |
| 2429 | 91-TL101-TIL-CD8+_CD103− | TIL | CD8+_CD103− | TGCGCCAGCAGCCAAGGAGACCGGGACTTCAACAATGAGCAGTTCTTC |
| 2430 | 91-TL101-TIL-CD8+_CD103− | TIL | CD8+_CD103− | TGCGCCAGCAGGGGACAGAGCTCCTACAATGAGCAGTTCTTC |
| 2431 | 91-TL101-TIL-CD8+_CD103− | TIL | CD8+_CD103− | TGTGCCAGCAGCGTGCCTTTAGCGGAGAGAAACATTCAGTACTTC |
| 2432 | 91-TL101-TIL-CD8+_CD103− | TIL | CD8+_CD103− | TGCAGTGCTAGAGTTTCGCTGGGGAGTAATGAAGCTTTCTTT |
| 2433 | 91-TL101-TIL-CD8+_CD103− | TIL | CD8+_CD103− | TGTGCCAGCAGTTATAGCACAGGGGGCGACTATGGCTACACCTTC |
| 2434 | 91-TL101-TIL-CD8+_CD103− | TIL | CD8+_CD103− | TGTGCCAGCAGACGACTAGAGTTTGGCGAGCAGTACTTC |
| 2435 | 91-TL101-TIL-CD8+_CD103− | TIL | CD8+_CD103− | TGTGCCAGCAGCCAAGATCTAGGGTATGGCTACACCTTC |
| 2436 | 91-TL101-TIL-CD8+_CD103− | TIL | CD8+_CD103− | TGTGCCAGCAGTTTATCAGGGCAGTTTAATCAGCCCCAGCATTTT |
| 2437 | 91-TL101-TIL-CD8+_CD103− | TIL | CD8+_CD103− | TGTGCCAGCAGTCGAACTCTCTTGGAGACCCAGTACTTC |
| 2438 | 91-TL101-TIL-CD8+_CD103− | TIL | CD8+_CD103− | TGTGCCAGCAGCGTGAATAGCAATCAGCCCCAGCATTTT |
| 2439 | 91-TL101-TIL-CD8+_CD103− | TIL | CD8+_CD103− | TGTGCCAGCAGTGAAACGCGAGCCGCGGGAGCAAATGAGCAGTTCTTC |
| 2440 | 91-TL101-TIL-CD8+_CD103− | TIL | CD8+_CD103− | TGTGCCAGCAGTTACAGGGGGGTTCCTGGCTACACCTTC |
| 2441 | 91-TL101-TIL-CD8+_CD103− | TIL | CD8+_CD103− | TGTGCCAGCAGTTCAACCGGTTCGGGGACCCCCTACGAGCAGTACTTC |
| 2442 | 91-TL101-TIL-CD8+_CD103− | TIL | CD8+_CD103− | TGTGCCAGCAGCGCGGGACCACCTAAGATCTACGAGCAGTACTTC |
| 2443 | 91-TL101-TIL-CD8+_CD103− | TIL | CD8+_CD103− | TGTGCCAGCAGCGTGGGGGTTGAGAATTCACCCCTCCACTTT |
| 2444 | 91-TL101-TIL-CD8+_CD103− | TIL | CD8+_CD103− | TGTGCCAGCAGCCTAGCGGGAGTGACTGGGGCCAACGTCCTGACTTTC |
| 2445 | 91-TL101-TIL-CD8+_CD103− | TIL | CD8+_CD103− | TGTGCCAGCAGTTTCACAGGGATTGATCAGCCCCAGCATTTT |
| 2446 | 91-TL101-TIL-CD8+_CD103− | TIL | CD8+_CD103− | TGCAGTGCTAGAGGGACTATCTACAATGAGCAGTTCTTC |
| 2447 | 91-TL101-TIL-CD8+_CD103− | TIL | CD8+_CD103− | TGTGCCAGCAGCTCCGGACAGTATAGCAATCAGCCCCAGCATTTT |
| 2448 | 91-TL101-TIL-CD8+_CD103− | TIL | CD8+_CD103− | TGTGCCACCAGCACGAGGTCCTCTAATGAAAAACTGTTTTTT |
| 2449 | 91-TL101-TIL-CD8+_CD103− | TIL | CD8+_CD103− | TGTGCCAGCAACCTTGGTGCTATCGGGGCCAACGTCCTGACTTTC |
| 2450 | 91-TL101-TIL-CD8+_CD103− | TIL | CD8+_CD103− | TGTGCCAGCTCACCGGACCTTCCCGACTCTGGAAACACCATATATTTT |
| 2451 | 91-TL101-TIL-CD8+_CD103− | TIL | CD8+_CD103− | TGTGCCAGCAGTGTCCGGACTGAAAAACTGTTTTTT |
| 2452 | 91-TL101-TIL-CD8+_CD103− | TIL | CD8+_CD103− | TGTGCCAGCTCTTGGGAAGAGACCCAGTACTTC |
| 2453 | 91-TL101-TIL-CD8+_CD103− | TIL | CD8+_CD103− | TGTGCCAGCAGTAGACAGGGGGGTTCGAATGGCTACACCTTC |
| 2454 | 91-TL101-TIL-CD8+_CD103− | TIL | CD8+_CD103− | TGTGCCAGCAGTTTATGGGGACAGGGGGCCCGGAACACTGAAGCTTTCTTT |
| 2455 | 91-TL101-TIL-CD8+_CD103− | TIL | CD8+_CD103− | TGTGCCAGCGGAGGGTTCGGGATGTCGGGCGAGCAGTACTTC |
| 2456 | 91-TL101-TIL-CD8+_CD103− | TIL | CD8+_CD103− | TGTGCCAGCAGCTTCTCGGTTGGAGTAGGAGGAACCGGGGAGCTGTTTTTT |
| 2457 | 91-TL101-TIL-CD8+_CD103− | TIL | CD8+_CD103− | TGTGCCAGCAGCCGGGACAGCCTAAAAGGGTACTTC |
| 2458 | 91-TL101-TIL-CD8+_CD103− | TIL | CD8+_CD103− | TGTGCCAGCAGTCCGGGACTAGTGAAAACCGGGGAGCTGTTTTTT |
| 2459 | 91-TL101-TIL-CD8+_CD103− | TIL | CD8+_CD103− | TGCGCCAGCACCACCGGGACAGGGGCGCTCGGGGCCAACGTCCTGACTTTC |
| 2460 | 91-TL101-TIL-CD8+_CD103− | TIL | CD8+_CD103− | TGTGCCAGCCGAAACGGACTAGCGGGAGGGCCTTCCTACGAGCAGTACTTC |
| 2461 | 91-TL101-TIL-CD8+_CD103− | TIL | CD8+_CD103− | TGTGCCAGCAGCTATGGCAGAGACACAGATACGCAGTATTTT |
| 2462 | 91-TL101-TIL-CD8+_CD103− | TIL | CD8+_CD103− | TGTGCCAGCAGCCCGACTCAAGGGACCCGAGCTTTCTTT |
| 2463 | 91-TL101-TIL-CD8+_CD103− | TIL | CD8+_CD103− | TGTGCCAGCAGCTTTCGGGGGGGATACAATGAGCAGTTCTTC |
| 2464 | 91-TL101-TIL-CD8+_CD103− | TIL | CD8+_CD103− | TGTGCCAGCACGACATTAGGCTCTGGGGCCAACGTCCTGACTTTC |
| 2465 | 91-TL101-TIL-CD8+_CD103− | TIL | CD8+_CD103− | TGTGCCACCAGCAGAGCCGGGAGCGAGCAGTACTTC |
| 2466 | 91-TL101-TIL-CD8+_CD103− | TIL | CD8+_CD103− | TGTGCCAGCAGTACCGAGGGAAATCAGCCCCAGCATTTT |
| 2467 | 91-TL101-TIL-CD8+_CD103− | TIL | CD8+_CD103− | TGTGCCAGCAGCGAGACGCTAGCGGGCAACAATGAGCAGTTCTTC |
| 2468 | 91-TL101-TIL-CD8+_CD103− | TIL | CD8+_CD103− | TGCAGTGAACAGGCGAGGACCGGGGAGCTGTTTTTT |
| 2469 | 91-TL101-TIL-CD8+_CD103− | TIL | CD8+_CD103− | TGTGCCAGCAGGGCAGAAACCTACGAGCAGTACTTC |
| 2470 | 91-TL101-TIL-CD8+_CD103− | TIL | CD8+_CD103− | TGTGCCATCAGTGACGACTTCGGCGGGAGTTCCTACGAGCAGTACTTC |
| 2471 | 91-TL101-TIL-CD8+_CD103− | TIL | CD8+_CD103− | TGCAGTGCTAGAGTCCTAGCGGGAGGGGTCAATGAGCAGTTCTTC |
| 2472 | 91-TL101-TIL-CD8+_CD103− | TIL | CD8+_CD103− | TGCAGTGCTAGGACCGGAGCGGGAGACCCCTACGAGCAGTACTTC |
| 2473 | 91-TL101-TIL-CD8+_CD103− | TIL | CD8+_CD103− | TGTGCCAGCTCACCCTGGACTGTCTCGAACACCGGGGAGCTGTTTTTT |
| 2474 | 91-TL101-TIL-CD8+_CD103− | TIL | CD8+_CD103− | TGTGCCTGGAGTGCCCTGGCTGGGGCTTTCTTT |
| 2475 | 91-TL101-TIL-CD8+_CD103− | TIL | CD8+_CD103− | TGCGCCAGCAGCTTGGGACCTGGACAGGGGGACTATGGCTACACCTTC |
| 2476 | 91-TL101-TIL-CD8+_CD103− | TIL | CD8+_CD103− | TGCAGTGCTAGAGATGCAGACTCGAACACCGGGGAGCTGTTTTTT |
| 2477 | 91-TL101-TIL-CD8+_CD103− | TIL | CD8+_CD103− | TGTGCCAGCATCATTATGGGGGCTGAAGCTTTCTTT |
| 2478 | 91-TL101-TIL-CD8+_CD103− | TIL | CD8+_CD103− | TGTGCCAGCAGTTCCTGGACTAGCGGGAGGGCCGACTTT |
| 2479 | 91-TL101-TIL-CD8+_CD103− | TIL | CD8+_CD103− | TGTGCCAGCAGCCCCCTAATGATATCAGGGGGACAGCAGTTCTTC |
| 2480 | 91-TL101-TIL-CD8+_CD103− | TIL | CD8+_CD103− | TGTGCCAGCAGCTTAAGACTAGGGGTTAGAGAGCAGTTCTTC |
| 2481 | 91-TL101-TIL-CD8+_CD103− | TIL | CD8+_CD103− | TGTGCCAGCAGCTTCCAGACAGATACGCAGTATTTT |
| 2482 | 91-TL101-TIL-CD8+_CD103− | TIL | CD8+_CD103− | TGTGCTAGCAGCCCAGTGCCCGGGACAGGGGAAGGGACCGGGGA |
| | | | | GCTGTTTTTT |
| 2483 | 91-TL101-TIL-CD8+_CD103− | TIL | CD8+_CD103− | TGCAGTGCTAGAGGTCCTGTTTCGGCAGGCCTAAATTCACCCCTCCACTTT |
| 2484 | 91-TL101-TIL-CD8+_CD103− | TIL | CD8+_CD103− | TGCGCCAGCCATACCGGGACAGGGGCCTACGAGCAGTACTTC |
| 2485 | 91-TL101-TIL-CD8+_CD103− | TIL | CD8+_CD103− | TGTGCCAGCAGCCTCGGTCAGGGTTTTAGTGAGCAGTACTTC |
| 2486 | 91-TL101-TIL-CD8+_CD103− | TIL | CD8+_CD103− | TGTGCCAGCAGCCAAGGATGGACAGGGGAATACGAGCAGTACTTC |
| 2487 | 91-TL101-TIL-CD8+_CD103− | TIL | CD8+_CD103− | TGTGCCAGCAGCTTCTCCCCGATAGGAGGCGGGGTTAATAACACT |
| | | | | GAAGCTTTCTTT |
| 2488 | 91-TL101-TIL-CD8+_CD103− | TIL | CD8+_CD103− | TGCAGCGTTGAAGAGACCAAAGAGAACTATGGCTACACCTTC |
| 2489 | 91-TL101-TIL-CD8+_CD103− | TIL | CD8+_CD103− | TGTGCCAGCATTGGGACAGGACACTACGAGCAGTACTTC |
| 2490 | 91-TL101-TIL-CD8+_CD103− | TIL | CD8+_CD103− | TGTGCCAGCGGTTCTAGTCTCGGTACGCAGTATTTT |
| 2491 | 91-TL101-TIL-CD8+_CD103− | TIL | CD8+_CD103− | TGTGCCAGCAGCGTCATTGGGACTAGCGTATACAATGAGCAGTTCTTC |
| 2492 | 91-TL101-TIL-CD8+_CD103− | TIL | CD8+_CD103− | TGTGCCAGCAGTTACTCGTGGCTAGCCAGGGGCTCATATAATT |
| | | | | CACCCCTCCACTTT |
| 2493 | 91-TL101-TIL-CD8+_CD103− | TIL | CD8+_CD103− | TGTGCCAGCAGTTTAGGCAGGGGGCATGGTCAGCCCCAGCATTTT |
| 2494 | 91-TL101-TIL-CD8+_CD103− | TIL | CD8+_CD103− | TGCGCCAGCAGTGATGTACAGAGCAATCAGCCCCAGCATTTT |
| 2495 | 91-TL101-TIL-CD8+_CD103− | TIL | CD8+_CD103− | TGTGCCAGCAGCTTGAGCCTTCCTGGAAACACCATATATTTT |
| 2496 | 91-TL101-TIL-CD8+_CD103− | TIL | CD8+_CD103− | TGTGCCAGCAGCCCTCAAGCGAGGATTAAACAAGAGACCCAGTACTTC |
| 2497 | 91-TL101-TIL-CD8+_CD103− | TIL | CD8+_CD103− | TGTGCCACCAGTGTGGAGGGGAATGAGCAGTTCTTC |
| 2498 | 91-TL101-TIL-CD8+_CD103− | TIL | CD8+_CD103− | TGTGCCAGCGAGGGACTAGCGGGAGGTGAGCAGTTCTTC |
| 2499 | 91-TL101-TIL-CD8+_CD103− | TIL | CD8+_CD103− | TGTGCCAGCAGGCCGACCTCAGGGGGCGGATCACCCCTCCACTTT |
| 2500 | 91-TL101-TIL-CD8+_CD103− | TIL | CD8+_CD103− | TGCAGCGCGGCACAGGGGGCAGGGCAGCCCCAGCATTTT |
| 2501 | 91-TL101-TIL-CD8+_CD103− | TIL | CD8+_CD103− | TGTGCCAGCAGTCCAGAAGGAGGGTTTGACCCAAATCAGCC |
| | | | | CCAGCATTTT |
| 2502 | 91-TL101-TIL-CD8+_CD103− | TIL | CD8+_CD103− | TGTGCCAGCAGGTCTACCGGGACAGGGCTCAATGAGCAGTTCTTC |
| 2503 | 91-TL101-TIL-CD8+_CD103− | TIL | CD8+_CD103− | TGTGCCAGCAGCTTAGCCCGCCAGGGGGACGAGCAGTACTTC |
| 2504 | 91-TL101-TIL-CD8+_CD103− | TIL | CD8+_CD103− | TGCAGTGCCTCCGTGGGAGCGGGAGTTGTAGAGACCCAGTACTTC |
| 2505 | 91-TL101-TIL-CD8+_CD103− | TIL | CD8+_CD103− | TGTGCCAGCAGCCACGTTGCGGTTACCGGGGAGCTGTTTTTT |
| 2506 | 91-TL101-TIL-CD8+_CD103− | TIL | CD8+_CD103− | TGCAGTGCTAGAGTGTGGGGGTCTAGCACAGATACGCAGTATTTT |
| 2507 | 91-TL101-TIL-CD8+_CD103− | TIL | CD8+_CD103− | TGTGCCAGCAGTCCTCGGACAGGGAAGACGGTCAATGAGCAGTTCTTC |
| 2508 | 91-TL101-TIL-CD8+_CD103− | TIL | CD8+_CD103− | TGCGCCAGCAGCTTGACCCTGGACATTACCTACGAGCAGTACTTC |
| 2509 | 91-TL101-TIL-CD8+_CD103− | TIL | CD8+_CD103− | TGTGCCAGCATAAACCGGAACACCGGGGAGCTGTTTTTT |
| 2510 | 91-TL101-TIL-CD8+_CD103− | TIL | CD8+_CD103− | TGCGCCACCTCGGGAGTTTTAAAGACCCAGTACTTC |
| 2511 | 91-TL101-TIL-CD8+_CD103− | TIL | CD8+_CD103− | TGTGCCACCAGCAGAGAGGACAGATCCTATAGCAATCAG |
| | | | | CCCCAGCATTTT |
| 2512 | 91-TL101-TIL-CD8+_CD103− | TIL | CD8+_CD103− | TGTGCCAGCAGCCCCAGCGGGAGCTACACCGGGGAGCTGTTTTTT |
| 2513 | 91-TL101-TIL-CD8+_CD103− | TIL | CD8+_CD103− | TGTGCCAGCAGCCCACGGATGGCCACAGATACGCAGTATTTT |
| 2514 | 91-TL101-TIL-CD8+_CD103− | TIL | CD8+_CD103− | TGTGCCAGCTCACCACCGAGGACTAGCGGGAGTTACACCGGG |
| | | | | GAGCTGTTTTTT |
| 2515 | 91-TL101-TIL-CD8+_CD103− | TIL | CD8+_CD103− | TGTGCCAGCAGACATTTGAGGGGTGTCTCCTACGAGCAGTACTTC |
| 2516 | 91-TL101-TIL-CD8+_CD103− | TIL | CD8+_CD103− | TGCAGTGCTGAAACAGACACAGATACGCAGTATTTT |
| 2517 | 91-TL101-TIL-CD8+_CD103− | TIL | CD8+_CD103− | TGTGCCAGCAGCACTCAGGGGTTCAGCACTGAAGCTTTCTTT |
| 2518 | 91-TL101-TIL-CD8+_CD103− | TIL | CD8+_CD103− | TGTGCCTGGAGCGGCGGAGGGGATCAGCCCCAGCATTTT |
| 2519 | 91-TL101-TIL-CD8+_CD103− | TIL | CD8+_CD103− | TGTGCCAGCAGATTAACCGGGACAAGTCTTAGCGAGCAGTACTTC |
| 2520 | 91-TL101-TIL-CD8+_CD103− | TIL | CD8+_CD103− | TGTGCCAGCAGTTACGGGTCAGGGGGAGAGACCCAGTACTTC |
| 2521 | 91-TL101-TIL-CD8+_CD103− | TIL | CD8+_CD103− | TGCAGCGTTGAAGAGAGGGCGTGGGGAATGAGTGAGACCCAGTACTTC |
| 2522 | 91-TL101-TIL-CD8+_CD103− | TIL | CD8+_CD103− | TGTGCCAGCAGTTCAACACTGACAGGCAACGAGCAGTACTTC |
| 2523 | 91-TL101-TIL-CD8+_CD103− | TIL | CD8+_CD103− | TGTGCCACAAGAGTGGGGGGGTCTCAAGAGACCCAGTACTTC |
| 2524 | 91-TL101-TIL-CD8+_CD103− | TIL | CD8+_CD103− | TGCAGTGCGCCGGGACTTTCTAACTATGGCTACACCTTC |
| 2525 | 91-TL101-TIL-CD8+_CD103− | TIL | CD8+_CD103− | TGTGCCAGCACCGTAACCCCGGGACAGGGGTACGAGCAGTACTTC |
| 2526 | 91-TL101-TIL-CD8+_CD103− | TIL | CD8+_CD103− | TGTGCCAGCAGCTTAAGTCGTATCTACAATGAGCAGTTCTTC |
| 2527 | 91-TL101-TIL-CD8+_CD103− | TIL | CD8+_CD103− | TGTGCCAGCAAATTTACGGTGTTGAACACCGGGGAGCTGTTTTTT |
| 2528 | 91-TL101-TIL-CD8+_CD103− | TIL | CD8+_CD103− | TGTGCCAGCAGCATAACCTCTCGCCCGTACAATGAGCAGTTCTTC |
| 2529 | 91-TL101-TIL-CD8+_CD103− | TIL | CD8+_CD103− | TGTGCCAGCAGCCAAGAGGACGGGGCCAACGTCCTGACTTTC |
| 2530 | 91-TL101-TIL-CD8+_CD103− | TIL | CD8+_CD103− | TGTGCCATCAATGCAGTGGGCACAGATACGCAGTATTTT |
| 2531 | 91-TL101-TIL-CD8+_CD103− | TIL | CD8+_CD103− | TGCGCCAGCAGCCACGTAGCAGATACGCAGTATTTT |
| 2532 | 91-TL101-TIL-CD8+_CD103− | TIL | CD8+_CD103− | TGTGCCAGCAGTCCCTTACAGGGCCAGGGCTACACCTTC |
| 2533 | 91-TL101-TIL-CD8+_CD103− | TIL | CD8+_CD103− | TGTGCCACCAGTGATGGGACTAGCGGATACGAGCAGTACTTC |
| 2534 | 91-TL101-TIL-CD8+_CD103− | TIL | CD8+_CD103− | TGCAGTGCAGGACTAGCGGGAGGGCCCAGCGGCCAACACAA |
| | | | | TGAGCAGTTCTTC |
| 2535 | 91-TL101-TIL-CD8+_CD103− | TIL | CD8+_CD103− | TGCAGTGCTAGTGACAATGAGCAGTTCTTC |
| 2536 | 91-TL101-TIL-CD8+_CD103− | TIL | CD8+_CD103− | TGTGCCAGCAGCGTAGCGCCCGGCGGGGAGCTGTTTTTT |
| 2537 | 91-TL101-TIL-CD8+_CD103− | TIL | CD8+_CD103− | TGTGCCACCAGTGATGGAGGGGTGGGAGAGAGTGAGCAGTTCTTC |
| 2538 | 91-TL101-TIL-CD8+_CD103− | TIL | CD8+_CD103− | TGCAGTGCTAGAGAACCCACAGGGGTGGACACCGGGGAGCTGTTTTTT |
| 2539 | 91-TL101-TIL-CD8+_CD103− | TIL | CD8+_CD103− | TGTGCCAGCAGTGAACGCCTCCAGTTCTTC |
| 2540 | 91-TL101-TIL-CD8+_CD103− | TIL | CD8+_CD103− | TGTGCCAGCAGCCCGAGACTTACCCTTGATGGAGATACGCAGTATTTT |
| 2541 | 91-TL101-TIL-CD8+_CD103− | TIL | CD8+_CD103− | TGCAGTGCCGCAAAAACAGGGAGCACCGGGGAGCTGTTTTTT |
| 2542 | 91-TL101-TIL-CD8+_CD103− | TIL | CD8+_CD103− | TGCAGCGTTGAAGAAGGTAGCGGGAGGCAAGAGACCCAGTACTTC |
| 2543 | 91-TL101-TIL-CD8+_CD103− | TIL | CD8+_CD103− | TGTGCCAGCAGTCGAGACACCTCGGAGCAGTTCTTC |
| 2544 | 91-TL101-TIL-CD8+_CD103− | TIL | CD8+_CD103− | TGTGCCAGCAGCCCTGGGGTAGCGGGATGGACCGGGGAGCTGTTTTTT |
| 2545 | 91-TL101-TIL-CD8+_CD103− | TIL | CD8+_CD103− | TGTGCCACCAGCAGCAGACTAGCGGGGGGGTACAATGAGCAGTTCTTC |
| 2546 | 91-TL101-TIL-CD8+_CD103− | TIL | CD8+_CD103− | TGCGCCAGCAGCTTGAGGGGGTGGAACACCGGGGAGCTGTTTTTT |
| 2547 | 91-TL101-TIL-CD8+_CD103− | TIL | CD8+_CD103− | TGTGCCAGCAGCTTAATGGGTGGGGGGAATGAGCAGTTCTTC |
| 2548 | 91-TL101-TIL-CD8+_CD103− | TIL | CD8+_CD103− | TGCAGTGCTAGAGATCTAGCGGGAGTAAACAATGAGCAGTTCTTC |
| 2549 | 91-TL101-TIL-CD8+_CD103− | TIL | CD8+_CD103− | TGTGCCAGCAGGGTCTCGATGGACGACGGTGAAAAACTGTTTTTT |
| 2550 | 91-TL101-TIL-CD8+_CD103− | TIL | CD8+_CD103− | TGTGCCAGCAGTCCACAGGGGCGGCAGCAAGAGACCCAGTACTTC |
| 2551 | 91-TL101-TIL-CD8+_CD103− | TIL | CD8+_CD103− | TGTGCCAGCAGCTCAGGGACAGGGAAGGGCCGAGAGACCCAGTACTTC |
| 2552 | 91-TL101-TIL-CD8+_CD103− | TIL | CD8+_CD103− | TGTGCCACCAGTGAGGGGGGCTCCTACAATGAGCAGTTCTTC |
| 2553 | 91-TL101-TIL-CD8+_CD103− | TIL | CD8+_CD103− | TGTGCCAGCAGCTCCCGGGACAGGGGACTCTACAATGAGCAGTTCTTC |
| 2554 | 91-TL101-TIL-CD8+_CD103− | TIL | CD8+_CD103− | TGTGCCAGCAGTTATTGCTTAATTGAAGCGGGAGAATGTGAGCAGTACTTC |
| 2555 | 91-TL101-TIL-CD8+_CD103− | TIL | CD8+_CD103− | TGCAGTGCTCCCTACAGAGATACGCAGTATTTT |
| 2556 | 91-TL101-TIL-CD8+_CD103− | TIL | CD8+_CD103− | TGCGCCAGCAGCTTGGACAGGCGTTCCTACACCTTC |
| 2557 | 91-TL101-TIL-CD8+_CD103− | TIL | CD8+_CD103− | TGTGCCAGCAAGACCGGACAGGGTGGCAATCAGCCCCAGCATTTT |
| 2558 | 91-TL101-TIL-CD8+_CD103− | TIL | CD8+_CD103− | TGTGCCAGCAGTTACGGTCCGGGAGAGACCCAGTACTTC |
| 2559 | 91-TL101-TIL-CD8+_CD103− | TIL | CD8+_CD103− | TGTGCCAGCAGTCCCCGGGACAGCGGATGGGGCCCGAGCAC |
| | | | | AGATACGCAGTATTTT |
| 2560 | 91-TL101-TIL-CD8+_CD103− | TIL | CD8+_CD103− | TGCGCCAGCAGCTTAACGAGCTCCTACAATGAGCAGTTCTTC |
| 2561 | 91-TL101-TIL-CD8+_CD103− | TIL | CD8+_CD103− | TGTGCCAGCAGCCCCCGGGAAGGGCAGTTCGAGCAGTACTTC |
| 2562 | 91-TL101-TIL-CD8+_CD103− | TIL | CD8+_CD103− | TGTGCCAGCAGCTGGATAGGGACAGCCAAAGCTTTCTTT |
| 2563 | 91-TL101-TIL-CD8+_CD103− | TIL | CD8+_CD103− | TGTGCCAGCAGCTTAGCAGGACTAGCGATGAACACCGGGGAGCTGTTTTTT |
| 2564 | 91-TL101-TIL-CD8+_CD103− | TIL | CD8+_CD103− | TGTGCCAGCAGTCTAGCCGGCGATACGCAGTATTTT |
| 2565 | 91-TL101-TIL-CD8+_CD103− | TIL | CD8+_CD103− | TGTGCCAGCAGTTACTCGGTCAGCACAGATACGCAGTATTTT |
| 2566 | 91-TL101-TIL-CD8+_CD103− | TIL | CD8+_CD103− | TGTGCCAGCAGCCATTACGGTACCCTGAACACTGAAGCTTTCTTT |
| 2567 | 91-TL101-TIL-CD8+_CD103− | TIL | CD8+_CD103− | TGTGCCAGCAGCGCCCGGGTAGCGGGATATTACGAGCAGTACTTC |
| 2568 | 91-TL101-TIL-CD8+_CD103− | TIL | CD8+_CD103− | TGCAGTGCTAGGGACACCCAATCCGAGCAGTACTTC |
| 2569 | 91-TL101-TIL-CD8+_CD103− | TIL | CD8+_CD103− | TGTGCCAGCAGGATTACTAGCGGGCCTTACGAGCAGTACTTC |
| 2570 | 91-TL101-TIL-CD8+_CD103− | TIL | CD8+_CD103− | TGTGCCAGCAGTCTGACCCAGGGAAATAATTCACCCCTCCACTTT |
| 2571 | 91-TL101-TIL-CD8+_CD103− | TIL | CD8+_CD103− | TGCGCCAGCAGTGAGGTTCCGGGAGGGGTTTATGGCTACACCTTC |
| 2572 | 91-TL101-TIL-CD8+_CD103− | TIL | CD8+_CD103− | TGTGCCAGCAGCCCTAGCGGGAGGACCTACGAGCAGTACTTC |
| 2573 | 91-TL101-TIL-CD8+_CD103− | TIL | CD8+_CD103− | TGTGCCAGCAGTTTATGGGGAGCCCTGGAGCAGTACTTC |
| 2574 | 91-TL101-TIL-CD8+_CD103− | TIL | CD8+_CD103− | TGTGCCAGTAGTATGCCGAACACTGAAGCTTTCTTT |
| 2575 | 91-TL101-TIL-CD8+_CD103− | TIL | CD8+_CD103− | TGTGCCAGCAGCCCGGGACTAGCGGGAGATCCCTTC |
| | | | | ACAGATACGCAGTATTTT |
| 2576 | 91-TL101-TIL-CD8+_CD103− | TIL | CD8+_CD103− | TGCAGCGTTGTGGGGCTACAAGAGACCCAGTACTTC |
| 2577 | 91-TL101-TIL-CD8+_CD103− | TIL | CD8+_CD103− | TGTGCCAGCAGTTCCAGGGGGCTTAGTTCCGATATG |
| | | | | AACACTGAAGCTTTCTTT |
| 2578 | 91-TL101-TIL-CD8+_CD103− | TIL | CD8+_CD103− | TGCAGCGTTGAAGGTGGGCTAGCGGGAGGGCCTAAG |
| | | | | TCCAAAAACATTCAGTACTTC |
| 2579 | 91-TL101-TIL-CD8+_CD103− | TIL | CD8+_CD103− | TGTGCCATCAGTGTCAGGGAGAACACCGGGGAGCTGTTTTTT |
| 2580 | 91-TL101-TIL-CD8+_CD103− | TIL | CD8+_CD103− | TGTGCCAGCAGCCAAAGACTAGCCCCCCAAGAGACCCAGTACTTC |
| 2581 | 91-TL101-TIL-CD8+_CD103− | TIL | CD8+_CD103− | TGTGCCAGCAGCCAAGAGTGGGGGCCCCAGCATTTT |
| 2582 | 91-TL101-TIL-CD8+_CD103− | TIL | CD8+_CD103− | TGCAGTGCCCAGGGGAGCGACTCCTACGAGCAGTACTTC |
| 2583 | 91-TL101-TIL-CD8+_CD103− | TIL | CD8+_CD103− | TGCAGCGTTTTGGGTTCCTACAATGAGCAGTTCTTC |
| 2584 | 91-TL101-TIL-CD8+_CD103− | TIL | CD8+_CD103− | TGTGCCAGCAATTTCAGGGCGCAGGTTATGAACACCGGGGAG |
| | | | | CTGTTTTTT |
| 2585 | 91-TL101-TIL-CD8+_CD103− | TIL | CD8+_CD103− | TGCAGTGCCAGCTCCGGGACAGGGTTTAACTATGGCTACACCTTC |
| 2586 | 91-TL101-TIL-CD8+_CD103− | TIL | CD8+_CD103− | TGTGCCAGCAGCCAAGGTCGGAACAGTATAAACTATGGCTACACCTTC |
| 2587 | 91-TL101-TIL-CD8+_CD103− | TIL | CD8+_CD103− | TGTGCCAGTCGCGGGGAGGGCACTGAAGCTTTCTTT |
| 2588 | 91-TL101-TIL-CD8+_CD103− | TIL | CD8+_CD103− | TGCGCCAGCAGCTTGGAAGCGGGAGGGCCGGCCGGGGAGCTGTTTTTT |
| 2589 | 91-TL101-TIL-CD8+_CD103− | TIL | CD8+_CD103− | TGTGCCAGCAGCTTAGGGGGACAGTCCTACGAGCAGTACTTC |
| 2590 | 91-TL101-TIL-CD8+_CD103− | TIL | CD8+_CD103− | TGTGCCAGCTCACGAGCGGGAGGGAGCACAGATACGCAGTATTTT |
| 2591 | 91-TL101-TIL-CD8+_CD103− | TIL | CD8+_CD103− | TGTGCCAGCAGTAAACAGGTTCTTAGCAATCAGCCCCAGCATTTT |
| 2592 | 91-TL101-TIL-CD8+_CD103− | TIL | CD8+_CD103− | TGCGCCAGCAGTGCCTCAAACAGGGGTGAAAGTGAAGCTTTCTTT |
| 2593 | 91-TL101-TIL-CD8+_CD103− | TIL | CD8+_CD103− | TGTGCCAGCAGTTACTCCGTCGCCACCGGGGAGCTGTTTTTT |
| 2594 | 91-TL101-TIL-CD8+_CD103− | TIL | CD8+_CD103− | TGCAGTGCGCACGATCAGGGGGCGGCGACCTACGAGCAGTACTTC |
| 2595 | 91-TL101-TIL-CD8+_CD103− | TIL | CD8+_CD103− | TGTGCCACCAGCAGAGCCGGGACAGACCTTTTCACAGATACG |
| | | | | CAGTATTTT |
| 2596 | 91-TL101-TIL-CD8+_CD103− | TIL | CD8+_CD103− | TGCAGTGCCTTGGGAACAGGGGTATGGGGTGAGCAGTTCTTC |
| 2597 | 91-TL101-TIL-CD8+_CD103− | TIL | CD8+_CD103− | TGCAGTGCTTGGACGAGGGGACCAAACTCCTACGAGCAGTACTTC |
| 2598 | 91-TL101-TIL-CD8+_CD103− | TIL | CD8+_CD103− | TGTGCCAGCAGCCAAGTACTGGCCAATGAGCAGTTCTTC |
| 2599 | 91-TL101-TIL-CD8+_CD103− | TIL | CD8+_CD103− | TGTGCCAGCAGTTATCGGCTTAGCTCCTACAATGAGCAGTTCTTC |
| 2600 | 91-TL101-TIL-CD8+_CD103− | TIL | CD8+_CD103− | TGTGCCATCAGTGACGCGGGGGAGCGAGCTTTCTTT |
| 2601 | 91-TL101-TIL-CD8+_CD103− | TIL | CD8+_CD103− | TGCGCCAGCAGCTTCGGAGGGACAAGACTGAACACTGAAGCTTTCTTT |
| 2602 | 91-TL101-TIL-CD8+_CD103− | TIL | CD8+_CD103− | TGTGCCAGCAGCCTGGGACTAGATACGCAGTATTTT |
| 2603 | 91-TL101-TIL-CD8+_CD103− | TIL | CD8+_CD103− | TGTGCCAGCAGCAAACCATGGAGTCAGGGATGGATTAACTA |
| | | | | TGGCTACACCTTC |
| 2604 | 91-TL101-TIL-CD8+_CD103− | TIL | CD8+_CD103− | TGCAGTGCTAGAGGGACGAGCTCCCAAGAGACCCAGTACTTC |
| 2605 | 91-TL101-TIL-CD8+_CD103− | TIL | CD8+_CD103− | TGCAGCGCGGGACTAGCGGGCACGAACACCGGGGAGCTGTTTTTT |
| 2606 | 91-TL101-TIL-CD8+_CD103− | TIL | CD8+_CD103− | TGTGCCAGCAGCCATCGGACTAGCGGGAGCCATGAGCAGTTCTTC |
| 2607 | 91-TL101-TIL-CD8+_CD103− | TIL | CD8+_CD103− | TGCGCCAGCAGCTTGGAAGGCGAGCAGCCCCAGCATTTT |
| 2608 | 91-TL101-TIL-CD8+_CD103− | TIL | CD8+_CD103− | TGTGCCAGCAGCTCAGACCGGGACGGCACTGAAGCTTTCTTT |
| 2609 | 91-TL101-TIL-CD8+_CD103− | TIL | CD8+_CD103− | TGCAGCGTTGGGGTCGGGGATACTAACTATGGCTACACCTTC |
| 2610 | 91-TL101-TIL-CD8+_CD103− | TIL | CD8+_CD103− | TGTGCCAGCAGGGGACCATCCTACGAGCAGTACTTC |
| 2611 | 91-TL101-TIL-CD8+_CD103− | TIL | CD8+_CD103− | TGTGCCAGCAGTAATTGGGACAGCCAAGAGACCCAGTACTTC |
| 2612 | 91-TL101-TIL-CD8+_CD103− | TIL | CD8+_CD103− | TGTGCCAGCAGGAAACAGGGTGCAACTAATGAAAAACTGTTTTTT |
| 2613 | 91-TL101-TIL-CD8+_CD103− | TIL | CD8+_CD103− | TGCAGTGCTTACCTCCCGGGCGGGACAGGTTATGGCTACACCTTC |
| 2614 | 91-TL101-TIL-CD8+_CD103− | TIL | CD8+_CD103− | TGTGCCAGCAGCTTATCGGGGGGCGAGCAGTACTTC |
| 2615 | 91-TL101-TIL-CD8+_CD103− | TIL | CD8+_CD103− | TGCAGTGCTAGGGGGATGGAGCGGGAGGACCAAGAGACCCAGTACTTC |
| 2616 | 91-TL101-TIL-CD8+_CD103− | TIL | CD8+_CD103− | TGCGCCAGCAGCCCTACTAGCGGTGAATACAATGAGCAGTTCTTC |
| 2617 | 91-TL101-TIL-CD8+_CD103− | TIL | CD8+_CD103− | TGTGCCAGCAGTGGAGCCAGGGATCATTATGAAAAACTGTTTTTT |
| 2618 | 91-TL101-TIL-CD8+_CD103− | TIL | CD8+_CD103− | TGTGCCAGCAGCGCCCGGACTGAGAGCTCCTACAATGAGCAGTTCTTC |
| 2619 | 91-TL101-TIL-CD8+_CD103− | TIL | CD8+_CD103− | TGTGCCAGCTCACCCACCCCAGCGGGAGGGACCTACAATGAGCAGTTCTTC |
| 2620 | 91-TL101-TIL-CD8+_CD103− | TIL | CD8+_CD103− | TGTGCCAGCAGGCGGGAGCAGGGCAATGAGCAGTTCTTC |
| 2621 | 91-TL101-TIL-CD8+_CD103− | TIL | CD8+_CD103− | TGTGCCATCAGGGGGGACAGGGGGGGCAATGAGCAGTTCTTC |
| 2622 | 91-TL101-TIL-CD8+_CD103− | TIL | CD8+_CD103− | TGTGCCAGCAGCCAGCAGGGGACCTTATGGACAGATACGCAGTATTTT |
| 2623 | 91-TL101-TIL-CD8+_CD103− | TIL | CD8+_CD103− | TGTGCCAGCTCACCATTGTTAACAGGGGTAAACTATGGCTACACCTTC |
| 2624 | 91-TL101-TIL-CD8+_CD103− | TIL | CD8+_CD103− | TGTGCCAGCAGCATTCGGACAGGGCCTAACACAGATACGCAGTATTTT |
| 2625 | 91-TL101-TIL-CD8+_CD103− | TIL | CD8+_CD103− | TGCAGTGCTAGAGTTTATTTAGGCAGTCAAGAGACCCAGTACTTC |
| 2626 | 91-TL101-TIL-CD8+_CD103− | TIL | CD8+_CD103− | TGTGCCACCCAGGGCCAACCCAACAATGAGCAGTTCTTC |
| 2627 | 91-TL101-TIL-CD8+_CD103− | TIL | CD8+_CD103− | TGCAGCGTTCAAACAGACAGACACAATGAGCAGTTCTTC |
| 2628 | 91-TL101-TIL-CD8+_CD103− | TIL | CD8+_CD103− | TGTGCCAGCTTGCGGACTAGCGGGCCCTACAATGAGCAGTTCTTC |
| 2629 | 91-TL101-TIL-CD8+_CD103− | TIL | CD8+_CD103− | TGTGCCAGTAGTATCAGAAGCTCCTACGAGCAGTACTTC |
| 2630 | 91-TL101-TIL-CD8+_CD103− | TIL | CD8+_CD103− | TGCAGCGTTGAAGGGGGCGGGGGAACGCAGTATTTT |
| 2631 | 91-TL101-TIL-CD8+_CD103− | TIL | CD8+_CD103− | TGTGCCAGCAGTTTCGAGCGGGAATACAATGAGCAGTTCTTC |
| 2632 | 91-TL101-TIL-CD8+_CD103− | TIL | CD8+_CD103− | TGTGCCAGCAGCCATGGACTAGCGGGAGTATACGAGCAGTACTTC |
| 2633 | 91-TL101-TIL-CD8+_CD103− | TIL | CD8+_CD103− | TGTGCCAGCAGCTTGGCTACGGTTAATTCACCCCTCCACTTT |
| 2634 | 91-TL101-TIL-CD8+_CD103− | TIL | CD8+_CD103− | TGTGCCAGCACCTCGGGCTATGGCTACACCTTC |
| 2635 | 91-TL101-TIL-CD8+_CD103− | TIL | CD8+_CD103− | TGCAGTGCTAGAGATTTCCGGGGGGCAAGTACATTGGATTC |
| | | | | ACCCCTCCACTTT |
| 2636 | 91-TL101-TIL-CD8+_CD103− | TIL | CD8+_CD103− | TGTGCCAGTAGTAGAGGCCCCGGGAATTCACCCCTCCACTTT |
| 2637 | 91-TL101-TIL-CD8+_CD103− | TIL | CD8+_CD103− | TGTGCCAGCAGCCCCCGAGGCACCTATGGCTACACCTTC |
| 2638 | 91-TL101-TIL-CD8+_CD103− | TIL | CD8+_CD103− | TGTGCCAGCAGCTTAGGACTACGGGACAATGAGCAGTTCTTC |
| 2639 | 91-TL101-TIL-CD8+_CD103− | TIL | CD8+_CD103− | TGTGCCAGCAGTGAAGCACGGGACAGGGGGTTACCATCGTTCTTC |
| 2640 | 91-TL101-TIL-CD8+_CD103− | TIL | CD8+_CD103− | TGTGCCAGCAGCCAAGATGATATATACGCGGGCTACACCTTC |
| 2641 | 91-TL101-TIL-CD8+_CD103− | TIL | CD8+_CD103− | TGCGCCAGCAGTCAAGTAGCGGGAGGGCGTCAAGATACGCAGTATTTT |
| 2642 | 91-TL101-TIL-CD8+_CD103− | TIL | CD8+_CD103− | TGCAGCGTGACGAATCCAGGGGGCTCCTACGAGCAGTACTTC |
| 2643 | 91-TL101-TIL-CD8+_CD103− | TIL | CD8+_CD103− | TGTGCCAGTAGTAACCGACAGGGTCCCGAGCAGTACTTC |
| 2644 | 91-TL101-TIL-CD8+_CD103− | TIL | CD8+_CD103− | TGCAGTGCTAGAGGAGGGGCCGGGACTCTATACAATGAGCAGTTCTTC |
| 2645 | 91-TL101-TIL-CD8+_CD103− | TIL | CD8+_CD103− | TGTGCCAGCAGGTCCACAGGAGCCAGGAATCAGCCCCAGCATTTT |
| 2646 | 91-TL101-TIL-CD8+_CD103− | TIL | CD8+_CD103− | TGTGCCAGCAGCCCCCGACTTACCGGGGAGCTGTTTTTT |
| 2647 | 91-TL101-TIL-CD8+_CD103− | TIL | CD8+_CD103− | TGTGCCAGCAGCTTATGGGACAGGAGGAACACTGAAGCTTTCTTT |
| 2648 | 91-TL101-TIL-CD8+_CD103− | TIL | CD8+_CD103− | TGTGCCAGCAGCGTGTCGGACTGGGCCGGGGAGCTGTTTTTT |
| 2649 | 91-TL101-TIL-CD8+_CD103− | TIL | CD8+_CD103− | TGTGCCAGCAGTTTCCTCCAGGCGGACACCGGGGAGCTGTTTTTT |
| 2650 | 91-TL101-TIL-CD8+_CD103− | TIL | CD8+_CD103− | TGTGCCTGGAGTAGCAACTCACAGGGCGGAGAAAAACTGTTTTTT |
| 2651 | 91-TL101-TIL-CD8+_CD103− | TIL | CD8+_CD103− | TGTGCCAGCAACGAACGGGGGACCTATAATTCACCCCTCCACTTT |
| 2652 | 91-TL101-TIL-CD8+_CD103− | TIL | CD8+_CD103− | TGCGCCAGCAGCCAAGTACTAGATCAGCCCCAGCATTTT |
| 2653 | 91-TL101-TIL-CD8+_CD103− | TIL | CD8+_CD103− | TGTGCCAGCAGCGTTGGTATGAACACTGAAGCTTTCTTT |
| 2654 | 91-TL101-TIL-CD8+_CD103− | TIL | CD8+_CD103− | TGTGCCAGCAGCCAGTTTATGAACACTGAAGCTTTCTTT |
| 2655 | 91-TL101-TIL-CD8+_CD103− | TIL | CD8+_CD103− | TGTGCCAGCAGCCCTCCGAACACTGAAGCTTTCTTT |
| 2656 | 91-TL101-TIL-CD8+_CD103− | TIL | CD8+_CD103− | TGTGCCAGCAGTTTAGATCAGCCCCAGCATTTT |
| 2657 | 91-TL101-TIL-CD8+_CD103− | TIL | CD8+_CD103− | TGTGCCAGCTCACCAGGACAGGGAACCACCATATATTTT |
| 2658 | 91-TL101-TIL-CD8+_CD103− | TIL | CD8+_CD103− | TGTGCCTGGGACAGGCCGAACACTGAAGCTTTCTTT |
| 2659 | 91-TL101-TIL-CD8+_CD103− | TIL | CD8+_CD103− | TGCGCCAGCAGCTTGGGGGCAGAGACCCAGTACTTC |
| 2660 | 91-TL101-TIL-CD8+_CD103− | TIL | CD8+_CD103− | TGTGCCAGCAGTCCCTGGACAGCCGGGAGCACTGAAGCTTTCTTT |
| 2661 | 91-TL101-TIL-CD8+_CD103− | TIL | CD8+_CD103− | TGTGCCAGCAGCTTCGCTACAGCACTGAACACTGAAGCTTTCTTT |
| 2662 | 91-TL101-TIL-CD8+_CD103− | TIL | CD8+_CD103− | TGTGCCAGCACCCTTACCGGGACAGGGGGGTTAGAGGTTAG |
| | | | | AAGCAAGCCCCAGCATTTT |
| 2663 | 91-TL101-TIL-CD8+_CD103− | TIL | CD8+_CD103− | TGTGCCAGCAGCGGTTCACCAGATACGCAGTATTTT |
| 2664 | 91-TL101-TIL-CD8+_CD103− | TIL | CD8+_CD103− | TGTGCCAGCAGCCAAGGCACAGGGTACTACGAGCAGTACTTC |
| 2665 | 91-TL101-TIL-CD8+_CD103− | TIL | CD8+_CD103− | TGTGCCAGCAGTGTGGCAGGAAGCACAGATACGCAGTATTTT |
| 2666 | 91-TL101-TIL-CD8+_CD103− | TIL | CD8+_CD103− | TGTGCCAGCAGCGTGGACAGGGGTTCCTACGAGCAGTACTTC |
| 2667 | 91-TL101-TIL-CD8+_CD103− | TIL | CD8+_CD103− | TGTGCCAGCAGGCAGGGGGCAATTGGCAATCAGCCCCAGCATTTT |
| 2668 | 91-TL101-TIL-CD8+_CD103− | TIL | CD8+_CD103− | TGTGCCAGCAGCGTGAGGGACCGAACCTACAATGAGCAGTTCTTC |
| 2669 | 91-TL101-TIL-CD8+_CD103− | TIL | CD8+_CD103− | TGTGCCAGCAGCCCAGAAACGGGGGGAACCGGGGAGCTGTTTTTT |
| 2670 | 91-TL101-TIL-CD8+_CD103− | TIL | CD8+_CD103− | TGTGCCAGCAGTTCTCGACAGGAGATCTACTATGGCTACACCTTC |
| 2671 | 91-TL101-TIL-CD8+_CD103− | TIL | CD8+_CD103− | TGTGCCAGCATTCTATTCCCGGGACAGCCGAGCTACGAGCAGTACTTC |
| 2672 | 91-TL101-TIL-CD8+_CD103− | TIL | CD8+_CD103− | TGCAGTGCTAGAGCTTACGACAGGGTCTACGAGCAGTACTTC |
| 2673 | 91-TL101-TIL-CD8+_CD103− | TIL | CD8+_CD103− | TGTGCCAGCAAGGGACTAGCCGAATATGAGCAGTTCTTC |
| 2674 | 91-TL101-TIL-CD8+_CD103− | TIL | CD8+_CD103− | TGTGCCATCACTAGGGGGTCATCCTACAATGAGCAGTTCTTC |
| 2675 | 91-TL101-TIL-CD8+_CD103− | TIL | CD8+_CD103− | TGCAGTGCTAGAGATACAGGGCCAAATCAGCCCCAGCATTTT |
| 2676 | 91-TL101-TIL-CD8+_CD103− | TIL | CD8+_CD103− | TGCGCCAGCAGCACGGGACAGGGGTACACTGAAGCTTTCTTT |
| 2677 | 91-TL101-TIL-CD8+_CD103− | TIL | CD8+_CD103− | TGTGCCAGCAGCCCCAGACAGGGGCGGAGCACAGATACGCAGTATTTT |
| 2678 | 91-TL101-TIL-CD8+_CD103− | TIL | CD8+_CD103− | TGTGCCAGCAGTTTAGACGGACAGGGCTTGTTCTATAATTCACCCCTC |
| | | | | CACTTT |
| 2679 | 91-TL101-TIL-CD8+_CD103− | TIL | CD8+_CD103− | TGTGCCAGCAGTCGAACACAGGGGAATCAGCCCCAGCATTTT |
| 2680 | 91-TL101-TIL-CD8+_CD103− | TIL | CD8+_CD103− | TGCGCCAGCAGTGAGACTAGCGCTAAAGAGACCCAGTACTTC |
| 2681 | 91-TL101-TIL-CD8+_CD103− | TIL | CD8+_CD103− | TGTGCCAGCAGTGAAACTAGCGTTAGCACAGATACGCAGTATTTT |
| 2682 | 91-TL101-TIL-CD8+_CD103− | TIL | CD8+_CD103− | TGTGCCAGCAACTCAGGCAAAGAGACCCAGTACTTC |
| 2683 | 91-TL101-TIL-CD8+_CD103− | TIL | CD8+_CD103− | TGTGCCAGTAGTATCGGTTCCAGGGGATTTTCAGATACGCAGTATTTT |
| 2684 | 91-TL101-TIL-CD8+_CD103− | TIL | CD8+_CD103− | TGTGCCTGGAGTGTACGGGGAATCAGGAACACTGAAGCTTTCTTT |
| 2685 | 91-TL101-TIL-CD8+_CD103− | TIL | CD8+_CD103− | TGTGCCAGCTCACCGCGGACAGGGGAAAAAACTGAAGCTTTCTTT |
| 2686 | 91-TL101-TIL-CD8+_CD103− | TIL | CD8+_CD103− | TGTGCCAGCAGCCGCTTAGTGGGGACTAGCGGGAGAAGCAC |
| | | | | AGATACGCAGTATTTT |
| 2687 | 91-TL101-TIL-CD8+_CD103− | TIL | CD8+_CD103− | TGTGCCAGCAGAGGCAGCTACGAGCAGTACTTC |
| 2688 | 91-TL101-TIL-CD8+_CD103− | TIL | CD8+_CD103− | TGTGCCAGCCATGCTTCTCCGGGACAGGGTCCCGCAGATACGCAGTATTTT |
| 2689 | 91-TL101-TIL-CD8+_CD103− | TIL | CD8+_CD103− | TGCAGCTCCGCGGGGTGGAACAATGAGCAGTTCTTC |
| 2690 | 91-TL101-TIL-CD8+_CD103− | TIL | CD8+_CD103− | TGTGCCAGCCACACAGATACGCAGTATTTT |
| 2691 | 91-TL101-TIL-CD8+_CD103− | TIL | CD8+_CD103− | TGTGCCAGCAGCTACCAGAATTCACCCCTCCACTTT |
| 2692 | 91-TL101-TIL-CD8+_CD103− | TIL | CD8+_CD103− | TGCGCCAGCAGCGCCTGGACAGGGGGATGGCGGAGCTCCTACAA |
| | | | | TGAGCAGTTCTTC |
| 2693 | 91-TL101-TIL-CD8+_CD103− | TIL | CD8+_CD103− | TGTGCCAGCAGCCTAGCGGGAGGGAGCACAGATACGCAGTATTTT |
| 2694 | 91-TL101-TIL-CD8+_CD103− | TIL | CD8+_CD103− | TGTGCCAGCAGTTTGGGGAAGTATCAGCCCCAGCATTTT |
| 2695 | 91-TL101-TIL-CD8+_CD103− | TIL | CD8+_CD103− | TGTGCCAGCAGGCCGGGACAGGGGATCTACAATGAGCAGTTCTTC |
| 2696 | 91-TL101-TIL-CD8+_CD103− | TIL | CD8+_CD103− | TGCAGCGTCTTCTCCGGGACACACTCCTACGAGCAGTACTTC |
| 2697 | 91-TL101-TIL-CD8+_CD103− | TIL | CD8+_CD103− | TGTGCCACTACTTGGCAGGGGGCCCCCTACAATGAGCAGTTCTTC |
| 2698 | 91-TL101-TIL-CD8+_CD103− | TIL | CD8+_CD103− | TGTGCCAGCAGCCAAGTCCGACGACACCGGTACCAAGAGACCCAGTACTTC |
| 2699 | 91-TL101-TIL-CD8+_CD103− | TIL | CD8+_CD103− | TGTGCCTGGAGGGCCGATAGGACAGGGCGGGATCCAACTGATACG |
| | | | | CAGTATTTT |
| 2700 | 91-TL101-TIL-CD8+_CD103− | TIL | CD8+_CD103− | TGTGCCAGCAGACAACGGGGCGGGAGCAGCTATGAGCAGTTCTTC |
| 2701 | 91-TL101-TIL-CD8+_CD103− | TIL | CD8+_CD103− | TGTGCCAGCTCACCACAAGGACAGGGGGCGGGCTATGGCTACACCTTC |
| 2702 | 91-TL101-TIL-CD8+_CD103− | TIL | CD8+_CD103− | TGCAGTGCTAGAGCCGCCGGGACAGGGCTGACTGAAGCTTTCTTT |
| 2703 | 91-TL101-TIL-CD8+_CD103− | TIL | CD8+_CD103− | TGTGCCAGCAGTTTTGGGCAGAATCAGCCCCAGCATTTT |
| 2704 | 91-TL101-TIL-CD8+_CD103− | TIL | CD8+_CD103− | TGTGCCACCAGTGATTTTAGAGGACTGAACACTGAAGCTTTCTTT |
| 2705 | 91-TL101-TIL-CD8+_CD103− | TIL | CD8+_CD103− | TGCGCCAGCAGGGGGTTGGCGAACACTGAAGCTTTCTTT |
| 2706 | 91-TL101-TIL-CD8+_CD103− | TIL | CD8+_CD103− | TGCGCCAGCAGCTTGTACGGGCCGGGGGATACGCAGTATTTT |
| 2707 | 91-TL101-TIL-CD8+_CD103− | TIL | CD8+_CD103− | TGTGCCAGCAGCCCCGGACTAACTCGGTACGAGCAGTACTTC |
| 2708 | 91-TL101-TIL-CD8+_CD103− | TIL | CD8+_CD103− | TGCGCCAGCAGCTGGGACAACCCCTCCTACAATGAGCAGTTCTTC |
| 2709 | 91-TL101-TIL-CD8+_CD103− | TIL | CD8+_CD103− | TGTGCCAGCAGCTTTGCGGGACAGGGAGGCAATGAGCAGTTCTTC |
| 2710 | 91-TL101-TIL-CD8+_CD103− | TIL | CD8+_CD103− | TGCAGTGCTAGGGGAAATGAAAAACTGTTTTTT |
| 2711 | 91-TL101-TIL-CD8+_CD103− | TIL | CD8+_CD103− | TGTGCCAGCTCACCGGGAAACAATCAGCCCCAGCATTTT |
| 2712 | 91-TL101-TIL-CD8+_CD103− | TIL | CD8+_CD103− | TGTGCCAGCAGTTTATCATACAGCGGGGACAACTTC |
| 2713 | 91-TL101-TIL-CD8+_CD103− | TIL | CD8+_CD103− | TGCAGCGTTGTCGGTCTAAGGGGCTTTGGCTACACCTTC |
| 2714 | 91-TL101-TIL-CD8+_CD103− | TIL | CD8+_CD103− | TGTGCCAGCTCACCCATCGCGTCAGGGAAAGAGACCCAGTACTTC |
| 2715 | 91-TL101-TIL-CD8+_CD103− | TIL | CD8+_CD103− | TGTGCCAGCAGTCAAGCATGGGCAGGTGAGCAGTTCTTC |
| 2716 | 91-TL101-TIL-CD8+_CD103− | TIL | CD8+_CD103− | TGTGCCAGCAGCTTAGTTGGGCGGATCTACGAGCAGTACTTC |
| 2717 | 91-TL101-TIL-CD8+_CD103− | TIL | CD8+_CD103− | TGTGCCAGCAGCTCTCGAGGACAGGGTGACGAGCAGTACTTC |
| 2718 | 91-TL101-TIL-CD8+_CD103− | TIL | CD8+_CD103− | TGCAGTGCTTGGCAGGGCGCGATGAGCAATCAGCCCCAGCATTTT |
| 2719 | 91-TL101-TIL-CD8+_CD103− | TIL | CD8+_CD103− | TGCGCCAGCAGCCCGGGTATTAGAGCTGAAAAACTGTTTTTT |
| 2720 | 91-TL101-TIL-CD8+_CD103− | TIL | CD8+_CD103− | TGTGCCAGCAGTTTAGGGACCGGGGAGCTGTTTTTT |
| 2721 | 91-TL101-TIL-CD8+_CD103− | TIL | CD8+_CD103− | TGTGCCAGCAGTTACTCGTTTTTGGAGCCCCAGCATTTT |
| 2722 | 91-TL101-TIL-CD8+_CD103− | TIL | CD8+_CD103− | TGTGCCAGCAGCGTCGCCAAGAACACCGGGGAGCTGTTTTTT |
| 2723 | 91-TL101-TIL-CD8+_CD103− | TIL | CD8+_CD103− | TGTGCCAGCAGCCTAGTAAGCTCTGGAAACACCATATATTTT |
| 2724 | 91-TL101-TIL-CD8+_CD103− | TIL | CD8+_CD103− | TGTGCCAGCAGTTTATTAACTCCCTCCGGTGAGCAGTTCTTC |
| 2725 | 91-TL101-TIL-CD8+_CD103− | TIL | CD8+_CD103− | TGTGCCAGCAAGCCCCTAGCGGGAGGAGGCAATGAGCAGTTCTTC |
| 2726 | 91-TL101-TIL-CD8+_CD103− | TIL | CD8+_CD103− | TGTGCCAGCAGTACGGGACAGGGGGCGGATGGCTACACCTTC |
| 2727 | 91-TL101-TIL-CD8+_CD103− | TIL | CD8+_CD103− | TGCAGTGCCACTGGGCTAGCGGGTTTCTCCTACGAGCAGTACTTC |
| 2728 | 91-TL101-TIL-CD8+_CD103− | TIL | CD8+_CD103− | TGTGCCAGCAGCTTAGGACTAGCGGGAGGCCGGCATGAGCAGTTCTTC |
| 2729 | 91-TL101-TIL-CD8+_CD103− | TIL | CD8+_CD103− | TGTGCCAGCAGTTTACAGGGACGAAGAGGCTACACCTTC |
| 2730 | 91-TL101-TIL-CD8+_CD103− | TIL | CD8+_CD103− | TGTGCCAGCAGCTTAGAGACTAGCGGGAGCGAGCAGTACTTC |
| 2731 | 91-TL101-TIL-CD8+_CD103− | TIL | CD8+_CD103− | TGTGCCAGCAGCTTACCCGCGGCGTCGTGGCGGGAGGGACTC |
| | | | | TACAATGAGCAGTTCTTC |
| 2732 | 91-TL101-TIL-CD8+_CD103− | TIL | CD8+_CD103− | TGTGCCACCGAGGCAGGCCCTGGGGCCAACGTCCTGACTTTC |
| 2733 | 91-TL101-TIL-CD8+_CD103− | TIL | CD8+_CD103− | TGTGCCAGCTCTGTAAGCGGAGCATACAATGAGCAGTTCTTC |
| 2734 | 91-TL101-TIL-CD8+_CD103− | TIL | CD8+_CD103− | TGTGCCAGCACGAGACAGGGATCGACCTACGAGCAGTACTTC |
| 2735 | 91-TL101-TIL-CD8+_CD103− | TIL | CD8+_CD103− | TGTGCCAGCAGCCCGGGACTACAGGAGACCCAGTACTTC |
| 2736 | 91-TL101-TIL-CD8+_CD103− | TIL | CD8+_CD103− | TGTGCCAGCAGCTTACTTGGCAAGCCTAATGAAAAACTGTTTTTT |
| 2737 | 91-TL101-TIL-CD8+_CD103− | TIL | CD8+_CD103− | TGTGCAAGCAGCAGGGCGCGGGAGATCTACAATGAGCAGTTCTTC |
| 2738 | 91-TL101-TIL-CD8+_CD103− | TIL | CD8+_CD103− | TGTGCCAGCAATGGACAGGGAAATTCTAAGCCCCAGCATTTT |
| 2739 | 91-TL101-TIL-CD8+_CD103− | TIL | CD8+_CD103− | TGTGCCAGCAGCTTAAGGGAGGACACTGAAGCTTTCTTT |
| 2740 | 91-TL101-TIL-CD8+_CD103− | TIL | CD8+_CD103− | TGTGCCAGCAGCCGCAGTTTCCGTGGCACAGATACGCAGTATTTT |
| 2741 | 91-TL101-TIL-CD8+_CD103− | TIL | CD8+_CD103− | TGTGCCAGCAGTGCAGAAACAGTGAACACTGAAGCTTTCTTT |
| 2742 | 91-TL101-TIL-CD8+_CD103− | TIL | CD8+_CD103− | TGTGCCAGCAGGGGGGTTGATAATGAGCAGTTCTTC |
| 2743 | 91-TL101-TIL-CD8+_CD103− | TIL | CD8+_CD103− | TGCAGTGCTAGAGACTTGGGACTAGCGGGAAAGGCCGGCGCC |
| | | | | GAGCAGTACTTC |
| 2744 | 91-TL101-TIL-CD8+_CD103− | TIL | CD8+_CD103− | TGTGCCAGCAGTGTAACCGGCGCCGAGCAGTACTTC |
| 2745 | 91-TL101-TIL-CD8+_CD103− | TIL | CD8+_CD103− | TGTGCCAGCAGCCAAGATGGCGGGAGGCCCAATCAGCCCCAGCATTTT |
| 2746 | 91-TL101-TIL-CD8+_CD103− | TIL | CD8+_CD103− | TGTGCCAGCAGACCCCTTAGCGGGAGGGCCGAGGGCACAGAT |
| | | | | ACGCAGTATTTT |
| 2747 | 91-TL101-TIL-CD8+_CD103− | TIL | CD8+_CD103− | TGTGCCATCAGTGAGGAGGGGACATATAACACTGAAGCTTTCTTT |
| 2748 | 91-TL101-TIL-CD8+_CD103− | TIL | CD8+_CD103− | TGCAGCGTTGTTGAAGGGATTGGAAACACCATATATTTT |
| 2749 | 91-TL101-TIL-CD8+_CD103− | TIL | CD8+_CD103− | TGTGCCAGCAGCCCAAGGACTAGCGGGAGGACTGTATACAATG |
| | | | | AGCAGTTCTTC |
| 2750 | 91-TL101-TIL-CD8+_CD103− | TIL | CD8+_CD103− | TGTGCTAGTGGTTTGGTTACAGAGAATCAGCCCCAGCATTTT |
| 2751 | 91-TL101-TIL-CD8+_CD103− | TIL | CD8+_CD103− | TGTGCCAGCAGCCAAGATACAGGGTTCGAGACCCAGTACTTC |
| 2752 | 91-TL101-TIL-CD8+_CD103− | TIL | CD8+_CD103− | TGTGCCAGCAGTTCGTCCCGGGACAGGGTTTTCTACGAGCAGTACTTC |
| 2753 | 91-TL101-TIL-CD8+_CD103− | TIL | CD8+_CD103− | TGTGCCAGCGCCGCCGTCCGGGACGCCCTCCCCTACGAGCAGTACTTC |
| 2754 | 91-TL101-TIL-CD8+_CD103− | TIL | CD8+_CD103− | TGTGCCACCAGTGATTTTACGGGCCGGCAGTTCTTC |
| 2755 | 91-TL101-TIL-CD8+_CD103− | TIL | CD8+_CD103− | TGTGCCACCAGTGATGGGACTAAACCTAGCACAGATACGCAGTATTTT |
| 2756 | 91-TL101-TIL-CD8+_CD103− | TIL | CD8+_CD103− | TGTGCCAGCAGTGATTCAAGGGGGGCCAAAAACATTCAGTACTTC |
| 2757 | 91-TL101-TIL-CD8+_CD103− | TIL | CD8+_CD103− | TGTGCCAGCAGCTTACTGGGCTCTGGAAACACCATATATTTT |
| 2758 | 91-TL101-TIL-CD8+_CD103− | TIL | CD8+_CD103− | TGTGCCAGCAGCGTAGGCGGCGCCGGGGAGCTGTTTTTT |
| 2759 | 91-TL101-TIL-CD8+_CD103− | TIL | CD8+_CD103− | TGTGCCAGCAGTTTCCCAGATTACAGACTAGCGGGAGAA |
| | | | | AACGATGAGCAGTTCTTC |
| 2760 | 91-TL101-TIL-CD8+_CD103− | TIL | CD8+_CD103− | TGTGCTACCTCAGGTCGGGGGGATCAGCCCCAGCATTTT |
| 2761 | 91-TL101-TIL-CD8+_CD103− | TIL | CD8+_CD103− | TGTGCCAGCAGACGAGCTGGTCGAGATGGTGAGCAGTACTTC |
| 2762 | 91-TL101-TIL-CD8+_CD103− | TIL | CD8+_CD103− | TGTGCCATCAGTGATTACACAGATACGCAGTATTTT |
| 2763 | 91-TL101-TIL-CD8+_CD103− | TIL | CD8+_CD103− | TGTGCCAGCAGCAGGGCGCGGGAGATCTACAATGAGCAGTTCTTC |
| 2764 | 91-TL101-TIL-CD8+_CD103− | TIL | CD8+_CD103− | TGTGCCAGCTCACCACTGGGACCTAACGAGCAGTACTTC |
| 2765 | 91-TL101-TIL-CD8+_CD103− | TIL | CD8+_CD103− | TGTGCCAGCAGCTTAGTAAGCACCGGGGAGCTGTTTTTT |
| 2766 | 91-TL101-TIL-CD8+_CD103− | TIL | CD8+_CD103− | TGTGCCAGCAGCCAGGGACAGGGGGCTGCGGAAGCTTTCTTT |
| 2767 | 91-TL101-TIL-CD8+_CD103− | TIL | CD8+_CD103− | TGTGCCAGCAGTGAAACAGGGCTTTCATATCAGCCCCAGCATTTT |
| 2768 | 91-TL101-TIL-CD8+_CD103− | TIL | CD8+_CD103− | TGCGCCAGCAGCTTGGAGACAGCGGAAAACATTCAGTACTTC |
| 2769 | 91-TL101-TIL-CD8+_CD103− | TIL | CD8+_CD103− | TGTGCCATCAGTGGTGCTGGCGAGCAGTACTTC |
| 2770 | 91-TL101-TIL-CD8+_CD103− | TIL | CD8+_CD103− | TGTGCCAGCAGTTTAGCGGGGGCCAAAAACATTCAGTACTTC |
| 2771 | 91-TL101-TIL-CD8+_CD103− | TIL | CD8+_CD103− | TGTGCCACCAGTGACCCGGGACTAGTCTCCTACAATGAGCAGTTCTTC |
| 2772 | 91-TL101-TIL-CD8+_CD103− | TIL | CD8+_CD103− | TGTGCCAGCAGCCCCCAAAGTAGCGGCACCGGGGAGCTGTTTTTT |
| 2773 | 91-TL101-TIL-CD8+_CD103− | TIL | CD8+_CD103− | TGTGCCAGCAGTGGGCAGCGTTCGGGCGGCTCCTACAATGAGCAGTTCTTC |
| 2774 | 91-TL101-TIL-CD8+_CD103− | TIL | CD8+_CD103− | TGTGCCAGCAGATTCGGACGGGCCACAGATACGCAGTATTTT |
| 2775 | 91-TL101-TIL-CD8+_CD103− | TIL | CD8+_CD103− | TGCGCCAGCAGCCCCTTTAGACAGGCGGTAACTTTCTTT |
| 2776 | 91-TL101-TIL-CD8+_CD103− | TIL | CD8+_CD103− | TGCAGTGCTAGAGGTGGTGAGGGCGGGGGTAGCAATCAGCCCCAGCATTTT |
| 2777 | 91-TL101-TIL-CD8+_CD103− | TIL | CD8+_CD103− | TGTGCCAGCTGCTCTCGGAACTTACCCTACAATGAGCAGTTCTTC |
| 2778 | 91-TL101-TIL-CD8+_CD103− | TIL | CD8+_CD103− | TGTGCCAGCAGCTTGGGATCCTCCCCCGACTATGGCTACACCTTC |
| 2779 | 91-TL101-TIL-CD8+_CD103− | TIL | CD8+_CD103− | TGCAGTGCTAGAGGGGGGTACAGGAACACTGAAGCTTTCTTT |
| 2780 | 91-TL101-TIL-CD8+_CD103− | TIL | CD8+_CD103− | TGTGCCAGCAGCTTGGCCGTGACTCACTACGAGCAGTACTTC |
| 2781 | 95-TL684-TIL-CD8+_CD103+ | TIL | CD8+_CD103+ | TGTGCCAGCAGCTTAGCTGCGGCCATGGAAGCTTTCTTT |
| 2782 | 95-TL684-TIL-CD8+_CD103+ | TIL | CD8+_CD103+ | TGTGCCAGCAGTACGACTCAGGGGGGGCACGAGCAGTACTTC |
| 2783 | 95-TL684-TIL-CD8+_CD103+ | TIL | CD8+_CD103+ | TGTGCCAGCAGCGTGGGGGGAGAAAATCAGCCCCAGCATTTT |
| 2784 | 95-TL684-TIL-CD8+_CD103+ | TIL | CD8+_CD103+ | TGTGCCAGCAGCCAAGGAGTAGAAGCCTACGAGCAGTACTTC |
| 2785 | 95-TL684-TIL-CD8+_CD103+ | TIL | CD8+_CD103+ | TGCGCCAGCAGCTTTGGGTCTAGCGCCTATGAGCAGTTCTTC |
| 2786 | 95-TL684-TIL-CD8+_CD103+ | TIL | CD8+_CD103+ | TGTGCCAGCAGACCCGACCTCAATGAGCAGTTCTTC |
| 2787 | 95-TL684-TIL-CD8+_CD103+ | TIL | CD8+_CD103+ | TGCGCCAGCAGCCAAGCCCAACCGGGACAGGGGGATGAAAAACTGTTTTTT |
| 2788 | 95-TL684-TIL-CD8+_CD103+ | TIL | CD8+_CD103+ | TGTGCCAGCAGTCAAAATGAAAAACTGTTTTTT |
| 2789 | 95-TL684-TIL-CD8+_CD103+ | TIL | CD8+_CD103+ | TGTGCCAGCAGCTCGGGACAGGGCCGTTTTGGCTACACCTTC |
| 2790 | 95-TL684-TIL-CD8+_CD103+ | TIL | CD8+_CD103+ | TGTGCCAGCAGACTAATTCTCCGAGGGGATCAGCCCCAGCATTTT |
| 2791 | 95-TL684-TIL-CD8+_CD103+ | TIL | CD8+_CD103+ | TGTGCCAGCAGTTTCTACAGGGGATATGGCTACACCTTC |
| 2792 | 95-TL684-TIL-CD8+_CD103+ | TIL | CD8+_CD103+ | TGCGCCAGCAGCCATGGGGGATCGAACACTGAAGCTTTCTTT |
| 2793 | 95-TL684-TIL-CD8+_CD103+ | TIL | CD8+_CD103+ | TGTGCCACCAGTGATGAGGACCAGAACACCGGGGAGCTGTTTTTT |
| 2794 | 95-TL684-TIL-CD8+_CD103+ | TIL | CD8+_CD103+ | TGCGCCAGCAGCCAAGGAGGGGAAGAGACCCAGTACTTC |
| 2795 | 95-TL684-TIL-CD8+_CD103+ | TIL | CD8+_CD103+ | TGCGCCAGCAGCCAAACGAATCAGGCTTCTGCGCAGTATTTT |
| 2796 | 95-TL684-TIL-CD8+_CD103+ | TIL | CD8+_CD103+ | TGTGCCAGCAGCCAGGAAGGATGGGGGCGCTCCTACAATGAGCAGTTCTTC |
| 2797 | 95-TL684-TIL-CD8+_CD103+ | TIL | CD8+_CD103+ | TGTGCCAGCAGCTTACGGCCGAACACTGAAGCTTTCTTT |
| 2798 | 95-TL684-TIL-CD8+_CD103+ | TIL | CD8+_CD103+ | TGTGCCACCAGTGATCCCCGCGAAGGGTTCTATAGCAATCAGCCCCAGCAT |
| | | | | TTT |
| 2799 | 95-TL684-TIL-CD8+_CD103+ | TIL | CD8+_CD103+ | TGTGCCACCCGAGGCGGGGCAGGCAATGAGCAGTTCTTC |
| 2800 | 95-TL684-TIL-CD8+_CD103+ | TIL | CD8+_CD103+ | TGTGCCAGCAGCGCACCCGGGACGTACACTGAAGCTTTCTTT |
| 2801 | 95-TL684-TIL-CD8+_CD103+ | TIL | CD8+_CD103+ | TGTGCCAGCAGCTTAGCGCTAAGGGGGGGCCTGCCTGGCTACACCTTC |
| 2802 | 95-TL684-TIL-CD8+_CD103+ | TIL | CD8+_CD103+ | TGTGCCAGCTCCAGGGACGGGAACTCCTACAATGAGCAGTTCTTC |
| 2803 | 95-TL684-TIL-CD8+_CD103+ | TIL | CD8+_CD103+ | TGTGCCAGCAGCCCGGGGAGCTCCTACAATGAGCAGTTCTTC |
| 2804 | 95-TL684-TIL-CD8+_CD103+ | TIL | CD8+_CD103+ | TGTGCCAGCAGCCTAGGGGCCGAGCGGGTGGATGAGCAGTTCTTC |
| 2805 | 95-TL684-TIL-CD8+_CD103+ | TIL | CD8+_CD103+ | TGTGCCAGCAGTCCGACTAGCGGGGCTACCAATGAGCAGTTCTTC |
| 2806 | 95-TL684-TIL-CD8+_CD103+ | TIL | CD8+_CD103+ | TGTGCCAGCAGCCCCACCCCCAGAGGGACCTTCACGTACAATGAG |
| | | | | CAGTTCTTC |
| 2807 | 95-TL684-TIL-CD8+_CD103+ | TIL | CD8+_CD103+ | TGTGCCAGCAGCATCCAGGGGGGAAGCAATCAGCCCCAGCATTTT |
| 2808 | 95-TL684-TIL-CD8+_CD103+ | TIL | CD8+_CD103+ | TGTGCCAGCAGCTTAGGGGTTTTTAAGTCGCCCCAGCATTTT |
| 2809 | 95-TL684-TIL-CD8+_CD103+ | TIL | CD8+_CD103+ | TGTGCCAGCAGTTTCCGGGACAGGGCTGATGGCTATGGCTACACCTTC |
| 2810 | 95-TL684-TIL-CD8+_CD103+ | TIL | CD8+_CD103+ | TGTGCCAGGACGGGCGAGACCGGGGAGCTGTTTTTT |
| 2811 | 95-TL684-TIL-CD8+_CD103+ | TIL | CD8+_CD103+ | TGCGCCAGCTTACAGCCTATCTATGGCTACACCTTC |
| 2812 | 95-TL684-TIL-CD8+_CD103+ | TIL | CD8+_CD103+ | TGCAGTCCTCGGGACTCATCTAGCACAGATACGCAGTATTTT |
| 2813 | 95-TL684-TIL-CD8+_CD103+ | TIL | CD8+_CD103+ | TGTGCCAGCACCCTATGGCTAGCGGGAGTTGATGAGCAGTTCTTC |
| 2814 | 95-TL684-TIL-CD8+_CD103+ | TIL | CD8+_CD103+ | TGTGCCAGTAGTTCCCGGACTAGCGGAGTTTCCTACGAGCAGTACTTC |
| 2815 | 95-TL684-TIL-CD8+_CD103+ | TIL | CD8+_CD103+ | TGTGCCACCAGTGATATACCGGACAGGGCCACTCTGAACACCG |
| | | | | GGGAGCTGTTTTTT |
| 2816 | 95-TL684-TIL-CD8+_CD103+ | TIL | CD8+_CD103+ | TGTGCCAGTAGTATAGCCCTCTGGGACTTCTACAATGAGCAGTTCTTC |
| 2817 | 95-TL684-TIL-CD8+_CD103+ | TIL | CD8+_CD103+ | TGCGCCAGCAGCCCCGACGGAGGGGGACGTTACGAGCAGTACTTC |
| 2818 | 95-TL684-TIL-CD8+_CD103+ | TIL | CD8+_CD103+ | TGCGCCAGCAGCTTCGACAGCTACGAGCAGTACTTC |
| 2819 | 95-TL684-TIL-CD8+_CD103+ | TIL | CD8+_CD103+ | TGTGCCAGCAGTTTTTTCTTTGCCACTGAAGCTTTCTTT |
| 2820 | 95-TL684-TIL-CD8+_CD103+ | TIL | CD8+_CD103+ | TGTGCCAGCAGCCCGGGACTAGCGGGAGAGCAGTTCTTC |
| 2821 | 95-TL684-TIL-CD8+_CD103+ | TIL | CD8+_CD103+ | TGTGCCAGCAGCGAATGGGGGGCCCGGGCTTTCTTT |
| 2822 | 95-TL684-TIL-CD8+_CD103+ | TIL | CD8+_CD103+ | TGTGCCAGCAACTTGCCGGGCACTAGCGGGTTATCCACA |
| | | | | GATACGCAGTATTTT |
| 2823 | 95-TL684-TIL-CD8+_CD103+ | TIL | CD8+_CD103+ | TGTGCCAGCAGTTACTCGGCCTTCTCCGAGGGGTTGAAC |
| | | | | ACTGAAGCTTTCTTT |
| 2824 | 95-TL684-TIL-CD8+_CD103+ | TIL | CD8+_CD103+ | TGTGCCAGCAGAGCTACTAGCACAGATACGCAGTATTTT |
| 2825 | 95-TL684-TIL-CD8+_CD103+ | TIL | CD8+_CD103+ | TGTGCCAGCAGAAGAAGGAGGGACGAGTATTCACCCCTCCACTTT |
| 2826 | 95-TL684-TIL-CD8+_CD103+ | TIL | CD8+_CD103+ | TGTGCCACCAGTGATTTAGTTGGGACTAGCGGGAGGACCTACAA |
| | | | | TGAGCAGTTCTTC |
| 2827 | 95-TL684-TIL-CD8+_CD103+ | TIL | CD8+_CD103+ | TGTGCCTGGAGTCTCGCGGACTACGAGCAGTACTTC |
| 2828 | 95-TL684-TIL-CD8+_CD103+ | TIL | CD8+_CD103+ | TGTGCCAGCAGTTCCGGGGGGAACACTGAAGCTTTCTTT |
| 2829 | 95-TL684-TIL-CD8+_CD103+ | TIL | CD8+_CD103+ | TGCAGTGCTAGAGACCGGACTAGCGGGGATTACAATGAGCAGTTCTTC |
| 2830 | 95-TL684-TIL-CD8+_CD103+ | TIL | CD8+_CD103+ | TGTGCCAGCAGCCCCCCGGCAAAGACCTACGAGCAGTACTTC |
| 2831 | 95-TL684-TIL-CD8+_CD103+ | TIL | CD8+_CD103+ | TGTGCCACCAGCAGAGATTTTCGCGGCGAGCAGTACTTC |
| 2832 | 95-TL684-TIL-CD8+_CD103+ | TIL | CD8+_CD103+ | TGTGCCTGGAGCCCAGAAAGGGATTCCTACGAGCAGTACTTC |
| 2833 | 95-TL684-TIL-CD8+_CD103+ | TIL | CD8+_CD103+ | TGTGCCAGCAGTGATAACGGGGGTGACACTGAAGCTTTCTTT |
| 2834 | 95-TL684-TIL-CD8+_CD103+ | TIL | CD8+_CD103+ | TGTGCCAGCAGTTTCCTCCAGGGGAATGAGCAGTTCTTC |
| 2835 | 95-TL684-TIL-CD8+_CD103+ | TIL | CD8+_CD103+ | TGTGCCAGCAGTCCCGCCGTGGGTGATAGGGAAAAACTGTTTTTT |
| 2836 | 95-TL684-TIL-CD8+_CD103+ | TIL | CD8+_CD103+ | TGTGCCAGCAGCTTAGGGAGGGTCGGTGAGCAGTTCTTC |
| 2837 | 95-TL684-TIL-CD8+_CD103+ | TIL | CD8+_CD103+ | TGTGCCAGCACCTCGGGGTCCCAGGTTGAGACCCAGTACTTC |
| 2838 | 95-TL684-TIL-CD8+_CD103+ | TIL | CD8+_CD103+ | TGTGCCAGCAGCAAAGGGACAGCTACCTACGAGCAGTACTTC |
| 2839 | 95-TL684-TIL-CD8+_CD103+ | TIL | CD8+_CD103+ | TGCGCCAGCAGCCAAGGGTATCTGGGGGCAGATACGCAGTATTTT |
| 2840 | 95-TL684-TIL-CD8+_CD103+ | TIL | CD8+_CD103+ | TGTGCCAGCAGTTTCAGGTCCGCCGGGGAGCTGTTTTTT |
| 2841 | 95-TL684-TIL-CD8+_CD103+ | TIL | CD8+_CD103+ | TGTGCCAGCAGTGAAGGGTCTAGCGGGGGGGACGAGCAGTACTTC |
| 2842 | 95-TL684-TIL-CD8+_CD103+ | TIL | CD8+_CD103+ | TGCGCCAGCAGCCAAGTCGCTTACTCCTACAATGAGCAGTTCTTC |
| 2843 | 95-TL684-TIL-CD8+_CD103+ | TIL | CD8+_CD103+ | TGTGCCAGCAGCTTACCTCAAGAGACCCAGTACTTC |
| 2844 | 95-TL684-TIL-CD8+_CD103+ | TIL | CD8+_CD103+ | TGTGCCAGCAGCTTTGGGACAGGGAGCTACGAGCAGTACTTC |
| 2845 | 95-TL684-TIL-CD8+_CD103+ | TIL | CD8+_CD103+ | TGTGCCAGCAGTTTTGCACCTTACAATGAGCAGTTCTTC |
| 2846 | 95-TL684-TIL-CD8+_CD103+ | TIL | CD8+_CD103+ | TGTGCCAGCAGCGGGACGGTGGGGAACACTGAAGCTTTCTTT |
| 2847 | 95-TL684-TIL-CD8+_CD103+ | TIL | CD8+_CD103+ | TGTGCCAGCAGGGGCGGGAATCAGCCCCAGCATTTT |
| 2848 | 95-TL684-TIL-CD8+_CD103+ | TIL | CD8+_CD103+ | TGTGCCGTGGGGTGGTTCTTC |
| 2849 | 95-TL684-TIL-CD8+_CD103+ | TIL | CD8+_CD103+ | TGTGCCAGCAGTTACTCGATCGGGGAGGATCAGCCCCAGCATTTT |
| 2850 | 95-TL684-TIL-CD8+_CD103+ | TIL | CD8+_CD103+ | TGTGCCAGCAGTTATGATAGGGGGAATTCACCCCTCCACTTT |
| 2851 | 95-TL684-TIL-CD8+_CD103+ | TIL | CD8+_CD103+ | TGTGCCTGGAGTCTTAGCCCAGACAGTGAAGCTTTCTTT |
| 2852 | 95-TL684-TIL-CD8+_CD103+ | TIL | CD8+_CD103+ | TGCGCCAGCAGCCAAGGTTCTGACAGGGTGACCTACGAGCAGTACTTC |
| 2853 | 95-TL684-TIL-CD8+_CD103+ | TIL | CD8+_CD103+ | TGTGCCAGCATGACAGGGGGTCAGTCACCCCTCCACTTT |
| 2854 | 95-TL684-TIL-CD8+_CD103+ | TIL | CD8+_CD103+ | TGTGCCAGCAGCTTAGGGGGGAAGGTGGGAGCTTTCTTT |
| 2855 | 95-TL684-TIL-CD8+_CD103+ | TIL | CD8+_CD103+ | TGTGCCAGCAGTGTCTACGCGGGACAGGGTTACGAGCAGTACTTC |
| 2856 | 95-TL684-TIL-CD8+_CD103+ | TIL | CD8+_CD103+ | TGTGCCAGCAGTTTATCGTTTGGGGACTACGAGCAGTACTTC |
| 2857 | 95-TL684-TIL-CD8+_CD103+ | TIL | CD8+_CD103+ | TGTGCCAGCAGCCAAGCTATGACAGGGGGCGCCGACTATGGC |
| | | | | TACACCTTC |
| 2858 | 95-TL684-TIL-CD8+_CD103+ | TIL | CD8+_CD103+ | TGTGCCAGCAGCTTGGGACAGGGCCAAGAGACCCAGTACTTC |
| 2859 | 95-TL684-TIL-CD8+_CD103+ | TIL | CD8+_CD103+ | TGTGCCACCAAGGACAGGGCGAGGACTGAAGCTTTCTTT |
| 2860 | 95-TL684-TIL-CD8+_CD103+ | TIL | CD8+_CD103+ | TGTGCCAGTAGTATAGCGGGAGGGCCGCGGAATGAGCAGTTCTTC |
| 2861 | 95-TL684-TIL-CD8+_CD103+ | TIL | CD8+_CD103+ | TGCGCCAGCAGCTTGTGGGGACTGGGAGCTGATGGCTACACCTTC |
| 2862 | 95-TL684-TIL-CD8+_CD103+ | TIL | CD8+_CD103+ | TGCGCCAGCAGCCACTCTCGGGAGCTGGAAGATACGCAGTATTTT |
| 2863 | 95-TL684-TIL-CD8+_CD103+ | TIL | CD8+_CD103+ | TGTGCCAGCAGCGCGTCCGGTACAAATCAGCCCCAGCATTTT |
| 2864 | 95-TL684-TIL-CD8+_CD103+ | TIL | CD8+_CD103+ | TGTGCCAGCAGTTCCTTAGCCGACAGAACTAGGGGCTACACCTTC |
| 2865 | 95-TL684-TIL-CD8+_CD103+ | TIL | CD8+_CD103+ | TGCGCCAGCAGCCAGGGACAACGGGCCTCCTACGAGCAGTACTTC |
| 2866 | 95-TL684-TIL-CD8+_CD103+ | TIL | CD8+_CD103+ | TGCGCCAGCAGCCTGAAAAGCTCCTACAATGAGCAGTTCTTC |
| 2867 | 95-TL684-TIL-CD8+_CD103+ | TIL | CD8+_CD103+ | TGTGCCAGCAGCGGGCGACAGGGGCACACTGAAGCTTTCTTT |
| 2868 | 95-TL684-TIL-CD8+_CD103+ | TIL | CD8+_CD103+ | TGTGCCAGCAAGGGGCGGAGCTGGCTCCTCTACGAGCAGTACTTC |
| 2869 | 95-TL684-TIL-CD8+_CD103+ | TIL | CD8+_CD103+ | TGTGCCAGCAGCCCGATGGGAGGGTTGAACACTGAAGCTTTCTTT |
| 2870 | 95-TL684-TIL-CD8+_CD103+ | TIL | CD8+_CD103+ | TGCAGTGCACTTAACAGGGGCCGCGGATACAATCAGCCCCAGCATTTT |
| 2871 | 95-TL684-TIL-CD8+_CD103+ | TIL | CD8+_CD103+ | TGTGCCTGGAGTACGATTGACAATGAGCAGTTCTTC |
| 2872 | 95-TL684-TIL-CD8+_CD103+ | TIL | CD8+_CD103+ | TGTGCCAGCAGCTTGGGGGGTCGGGAGCAGTTCTTC |
| 2873 | 95-TL684-TIL-CD8+_CD103+ | TIL | CD8+_CD103+ | TGTGCCTCCCTCGCGAGCCGCAATATTCAAGAGACCCAGTACTTC |
| 2874 | 95-TL684-TIL-CD8+_CD103+ | TIL | CD8+_CD103+ | TGCGCCAGCAGCTTGGCAAGTACTGAAGCTTTCTTT |
| 2875 | 95-TL684-TIL-CD8+_CD103+ | TIL | CD8+_CD103+ | TGTGCCAGCAGCTTAGGGCCGGGACAGGGGGCCTACGAGCAGTACTTC |
| 2876 | 95-TL684-TIL-CD8+_CD103+ | TIL | CD8+_CD103+ | TGTGCCATCAGCCAACTAGCGGGTAAAGAGACCCAGTACTTC |
| 2877 | 95-TL684-TIL-CD8+_CD103+ | TIL | CD8+_CD103+ | TGTGCCAGCAGCACCCGACAGAGCAAAGCGGAGACCCAGTACTTC |
| 2878 | 95-TL684-TIL-CD8+_CD103+ | TIL | CD8+_CD103+ | TGTGCCACCAGTGATTTAGGAGGGACCCAGCATTTT |
| 2879 | 95-TL684-TIL-CD8+_CD103+ | TIL | CD8+_CD103+ | TGTGCCATCAGTGAGGGGTACGAGCAGTACTTC |
| 2880 | 95-TL684-TIL-CD8+_CD103+ | TIL | CD8+_CD103+ | TGTGCCAGCAGCCAGGAAGGGTGGAAGTACGAGCAGTACTTC |
| 2881 | 95-TL684-TIL-CD8+_CD103+ | TIL | CD8+_CD103+ | TGTGCCAGCAGCTTATTTGATGAGGAAGAGACCCAGTACTTC |
| 2882 | 95-TL684-TIL-CD8+_CD103+ | TIL | CD8+_CD103+ | TGTGCCAGCCAGGGACAGGGGGCTGGTTCACCCCTCCACTTT |
| 2883 | 95-TL684-TIL-CD8+_CD103+ | TIL | CD8+_CD103+ | TGCGCCAGCAGCCAAGCTTCGGGACTAGTCTTGAACACCGGG |
| | | | | GAGCTGTTTTTT |
| 2884 | 96-TL684-TIL-CD8+_CD103− | TIL | CD8+_CD103− | TGTGCCAGCAGTTACTCGCCGTGGGGGGATACGCAGTATTTT |
| 2885 | 96-TL684-TIL-CD8+_CD103− | TIL | CD8+_CD103− | TGTGCCAGCAGTTATACGGGAAGGTACGAGCAGTACTTC |
| 2886 | 96-TL684-TIL-CD8+_CD103− | TIL | CD8+_CD103− | TGCAGCGTTGTTCCGGGAGTAGGGTACGAGCAGTACTTC |
| 2887 | 96-TL684-TIL-CD8+_CD103− | TIL | CD8+_CD103− | TGTGCCAGCAGTTTATTTTGGGGCTCGAACACTGAAGCTTTCTTT |
| 2888 | 96-TL684-TIL-CD8+_CD103− | TIL | CD8+_CD103− | TGTGCCAGCAGCTTGGCCGCCAGTTCCCGATGGAATGAGCAGTTCTTC |
| 2889 | 96-TL684-TIL-CD8+_CD103− | TIL | CD8+_CD103− | TGTGCCAGCAGTTTACAAGGAGATACGCAGTATTTT |
| 2890 | 96-TL684-TIL-CD8+_CD103− | TIL | CD8+_CD103− | TGTGCCAGCAGTTTGGGGGTCCATGAGCAGTTCTTC |
| 2891 | 96-TL684-TIL-CD8+_CD103− | TIL | CD8+_CD103− | TGCAGTGCTAGAGCAAATCTACCCACAGATACGCAGTATTTT |
| 2892 | 96-TL684-TIL-CD8+_CD103− | TIL | CD8+_CD103− | TGTGCCAGCAGTGACGGAGGGGACACAGATACGCAGTATTTT |
| 2893 | 96-TL684-TIL-CD8+_CD103− | TIL | CD8+_CD103− | TGTGCCAGCAGCCAATCTGTTTCCTACGAGCAGTACTTC |
| 2894 | 96-TL684-TIL-CD8+_CD103− | TIL | CD8+_CD103− | TGCGCCAGCAGCCATGCCTCCTCTGGAAACACCATATATTTT |
| 2895 | 96-TL684-TIL-CD8+_CD103− | TIL | CD8+_CD103− | TGCGCCAGCAGCCAAGAATTAGGGGGGCAGCCCCAGCATTTT |
| 2896 | 96-TL684-TIL-CD8+_CD103− | TIL | CD8+_CD103− | TGTGCCAGCAGTTCGCGGCCCACAGATACGCAGTATTTT |
| 2897 | 96-TL684-TIL-CD8+_CD103− | TIL | CD8+_CD103− | TGTGCCAGCAGCACCAGCCCAGGGGGCCGGGGCTACACCTTC |
| 2898 | 96-TL684-TIL-CD8+_CD103− | TIL | CD8+_CD103− | TGTGCCAGCAGTTACTCGCCGGAGGATTCACCCCTCCACTTT |
| 2899 | 96-TL684-TIL-CD8+_CD103− | TIL | CD8+_CD103− | TGTGCCAGCAGTTTAGGTTTACAGGCCAATTATGGCTACACCTTC |
| 2900 | 96-TL684-TIL-CD8+_CD103− | TIL | CD8+_CD103− | TGTGCCAGCAGTTACTCGCGATCAGGGCCTCAGGGCTACACCTTC |
| 2901 | 96-TL684-TIL-CD8+_CD103− | TIL | CD8+_CD103− | TGTGCCAGCAGTGAAGCACTAGCACATGAGCAGTTCTTC |
| 2902 | 96-TL684-TIL-CD8+_CD103− | TIL | CD8+_CD103− | TGTGCCAGCAGTTCCCATAGGACAGGGTCAGATCAGCCCCAGCATTTT |
| 2903 | 96-TL684-TIL-CD8+_CD103− | TIL | CD8+_CD103− | TGTGCCAGCAGTACAGGAGGGGTCGAGCAGTACTTC |
| 2904 | 96-TL684-TIL-CD8+_CD103− | TIL | CD8+_CD103− | TGTGCCAGCAGCCAAGATCTAGCGGGAGGGCCTAGCACAGATACG |
| | | | | CAGTATTTT |
| 2905 | 96-TL684-TIL-CD8+_CD103− | TIL | CD8+_CD103− | TGTGCCAGCAGTTTCTACAGGGGATATGGCTACACCTTC |
| 2906 | 96-TL684-TIL-CD8+_CD103− | TIL | CD8+_CD103− | TGCAGTGCTAGAGATTCGGGAGCTGAAGCTTTCTTT |
| 2907 | 96-TL684-TIL-CD8+_CD103− | TIL | CD8+_CD103− | TGTGCCAGCAGCTTAATGGGGGGCCTGAACACTGAAGCTTTCTTT |
| 2908 | 96-TL684-TIL-CD8+_CD103− | TIL | CD8+_CD103− | TGTGCCAGCAGCCAGACCGGGAGCGGTCCCTACGAGCAGTACTTC |
| 2909 | 96-TL684-TIL-CD8+_CD103− | TIL | CD8+_CD103− | TGTGCCAGCAGGCTAACGTGGACAGATACCTACGAGCAGTACTTC |
| 2910 | 96-TL684-TIL-CD8+_CD103− | TIL | CD8+_CD103− | TGTGCCACCAGTGAGACTGGCAGTCACTACAATGAGCAGTTCTTC |
| 2911 | 96-TL684-TIL-CD8+_CD103− | TIL | CD8+_CD103− | TGCAGCGCTAAAAGACTAGCGGTCTACAATGAGCAGTTCTTC |
| 2912 | 96-TL684-TIL-CD8+_CD103− | TIL | CD8+_CD103− | TGTGCCAGCAGTTACCTGGGGCAAGAGACCCAGTACTTC |
| 2913 | 96-TL684-TIL-CD8+_CD103− | TIL | CD8+_CD103− | TGTGCCAGCAGCCAAGAAGGACATCAAGAGACCCAGTACTTC |
| 2914 | 96-TL684-TIL-CD8+_CD103− | TIL | CD8+_CD103− | TGTGCCAGCAACCAGGGGCTAGGCACTGAAGCTTTCTTT |
| 2915 | 96-TL684-TIL-CD8+_CD103− | TIL | CD8+_CD103− | TGCGCCAGCAGCCAAGAGGAGACCCAGTACTTC |
| 2916 | 96-TL684-TIL-CD8+_CD103− | TIL | CD8+_CD103− | TGCGCCAGCAGCACTTTATGGGGGGGGCAGTACAATGAGCAGTTCTTC |
| 2917 | 96-TL684-TIL-CD8+_CD103− | TIL | CD8+_CD103− | TGTGCCAGCAGTTACTCTTTCCAGACTGAAGCTTTCTTT |
| 2918 | 96-TL684-TIL-CD8+_CD103− | TIL | CD8+_CD103− | TGTGCCAGCAGCCAATATGGGCAGGGGGCGGCAACTAAT |
| | | | | GAAAAACTGTTTTTT |
| 2919 | 96-TL684-TIL-CD8+_CD103− | TIL | CD8+_CD103− | TGTGCCAGCAAGGACAGATGGGACATGAACACTGAAGCTTTCTTT |
| 2920 | 96-TL684-TIL-CD8+_CD103− | TIL | CD8+_CD103− | TGTGCCAGCAGCTTGGGGGACGGTGAGCAGTTCTTC |
| 2921 | 96-TL684-TIL-CD8+_CD103− | TIL | CD8+_CD103− | TGTGCCAGCAGTTATTCCGGGGGGAATGAGCAGTTCTTC |
| 2922 | 96-TL684-TIL-CD8+_CD103− | TIL | CD8+_CD103− | TGTGCCAGCAGTTACAGGGGCTCCAATCAGCCCCAGCATTTT |
| 2923 | 96-TL684-TIL-CD8+_CD103− | TIL | CD8+_CD103− | TGTGCCAGCAGCACAGGGGGGAACACTGAAGCTTTCTTT |
| 2924 | 96-TL684-TIL-CD8+_CD103− | TIL | CD8+_CD103− | TGCGCCAGCAGCCAAGCGACAGGGGGGTACAATGAGCAGTTCTTC |
| 2925 | 96-TL684-TIL-CD8+_CD103− | TIL | CD8+_CD103− | TGTGCCAGCAGCTTACTAGGGGGGACTAGCGGGAGGAATGA |
| | | | | GCAGTTCTTC |
| 2926 | 96-TL684-TIL-CD8+_CD103− | TIL | CD8+_CD103− | TGTGCCAGCAGTTACGGTTTTAGCGATGAGCAGTTCTTC |
| 2927 | 96-TL684-TIL-CD8+_CD103− | TIL | CD8+_CD103− | TGCGCCAGCAGCTTAAACAGGGCAAATGAGCAGTTCTTC |
| 2928 | 96-TL684-TIL-CD8+_CD103− | TIL | CD8+_CD103− | TGTGCCAGCAGCGCACCCGGGACGTACACTGAAGCTTTCTTT |
| 2929 | 96-TL684-TIL-CD8+_CD103− | TIL | CD8+_CD103− | TGTGCCAGCAGTAGGGGGGCGTGGGGGGCGAACACTGAAGCTTTCTTT |
| 2930 | 96-TL684-TIL-CD8+_CD103− | TIL | CD8+_CD103− | TGTGCCAGCAGCTTTTGGGCTAGCGGACGAGAGACCCAGTACTTC |
| 2931 | 96-TL684-TIL-CD8+_CD103− | TIL | CD8+_CD103− | TGCGCCAGCAGCCAAGATCACGGGGTCACCTACGAGCAGTACTTC |
| 2932 | 96-TL684-TIL-CD8+_CD103− | TIL | CD8+_CD103− | TGCGCCAGCAGCCACGTCGGGGACGGCTACACCTTC |
| 2933 | 96-TL684-TIL-CD8+_CD103− | TIL | CD8+_CD103− | TGTGCCACCAGCAGAGATCGCACAGAGAATTCACCCCTCCACTTT |
| 2934 | 96-TL684-TIL-CD8+_CD103− | TIL | CD8+_CD103− | TGTGCCATCAGCGCTAGCGGGGGGGCAGGGTACAATGAGCAGTTCTTC |
| 2935 | 96-TL684-TIL-CD8+_CD103− | TIL | CD8+_CD103− | TGTGCCAGCAGCCCAGATCAAAGGGGTGACCTAAATGAGCAGTTCTTC |
| 2936 | 96-TL684-TIL-CD8+_CD103− | TIL | CD8+_CD103− | TGTGCCAGCAGCCCTGAGGACGGTCGGAATGAAAAACTGTTTTTT |
| 2937 | 96-TL684-TIL-CD8+_CD103− | TIL | CD8+_CD103− | TGTGCCAGCAGTGAAGCGCGTACAGGACTCCAAGAGACCCAGTACTTC |
| 2938 | 96-TL684-TIL-CD8+_CD103− | TIL | CD8+_CD103− | TGCAGCGTCAGAAGGGGTCGGTACGAGCAGTACTTC |
| 2939 | 96-TL684-TIL-CD8+_CD103− | TIL | CD8+_CD103− | TGCAGCGTTGAGGGATGGTCGGTCAATGAGCAGTTCTTC |
| 2940 | 96-TL684-TIL-CD8+_CD103− | TIL | CD8+_CD103− | TGTGCCAGCAGCTTAGGTCGGGGAGAGGGTCAGCCCCAGCATTTT |
| 2941 | 96-TL684-TIL-CD8+_CD103− | TIL | CD8+_CD103− | TGTGCCAGCAGCTTAGACGGAGTCTCGGAGCAGTACTTC |
| 2942 | 96-TL684-TIL-CD8+_CD103− | TIL | CD8+_CD103− | TGTGCCAGCAGCCACATGACAAACTTCGACTCTGGAAACACCATATATTTT |
| 2943 | 96-TL684-TIL-CD8+_CD103− | TIL | CD8+_CD103− | TGCAGCGTTGAAGAAGGGCTAGCGGGAGGAGGAGTAGATACGCAGTATTTT |
| 2944 | 96-TL684-TIL-CD8+_CD103− | TIL | CD8+_CD103− | TGTGCCAGCAGTACCCCTGGGACAGGGGGATACGAGCAGTACTTC |
| 2945 | 96-TL684-TIL-CD8+_CD103− | TIL | CD8+_CD103− | TGTGCCAGCAGCTTGGTAGGCGAAGAGACCCAGTACTTC |
| 2946 | 96-TL684-TIL-CD8+_CD103− | TIL | CD8+_CD103− | TGTGCCAGCAGTTCTGGACCTGACAGGGCCGAGCAGTACTTC |
| 2947 | 96-TL684-TIL-CD8+_CD103− | TIL | CD8+_CD103− | TGTGCCAGCAGCTCAGGACTAGCGGGGCCCCCCAATGAGCAGTTCTTC |
| 2948 | 96-TL684-TIL-CD8+_CD103− | TIL | CD8+_CD103− | TGCAGTGCTAGACCCGCGGGGGGCCCTTACAATGAGCAGTTCTTC |
| 2949 | 96-TL684-TIL-CD8+_CD103− | TIL | CD8+_CD103− | TGTGCCAGCAGCCTAGGGGCCGAGCGGGTGGATGAGCAGTTCTTC |
| 2950 | 96-TL684-TIL-CD8+_CD103− | TIL | CD8+_CD103− | TGTGCCAGCAGCCCAAGATTGGTCTTGACAGGCCCTAATGAAAAA |
| | | | | CTGTTTTTT |
| 2951 | 96-TL684-TIL-CD8+_CD103− | TIL | CD8+_CD103− | TGTGCCAGCAGCTCCTCGGACAGGGGAGTGGCCGGGGAGCTGTTTTTT |
| 2952 | 96-TL684-TIL-CD8+_CD103− | TIL | CD8+_CD103− | TGTGCCAGCAGTTACTCGACAGGGGGGGTTAGTACCGGGGAGC |
| | | | | TGTTTTTT |
| 2953 | 96-TL684-TIL-CD8+_CD103− | TIL | CD8+_CD103− | TGTGCCACCAGCAGATCGACAGGGGGGGGCACTGAAGCTTTCTTT |
| 2954 | 96-TL684-TIL-CD8+_CD103− | TIL | CD8+_CD103− | TGTGCCAGCAGCTCCGTCCTGGGACAGAAGGGTAAAGAGACCCAGTACTTC |
| 2955 | 96-TL684-TIL-CD8+_CD103− | TIL | CD8+_CD103− | TGTGCCAGCAGCCCGAGGGGACTCAGCTCCTACAATGAGCAGTTCTTC |
| 2956 | 96-TL684-TIL-CD8+_CD103− | TIL | CD8+_CD103− | TGTGCCAGCAGCTGGACAGGAGTCACCGGGGAGCTGTTTTTT |
| 2957 | 96-TL684-TIL-CD8+_CD103− | TIL | CD8+_CD103− | TGTGCCAGCAGCTTGCGGGGACCTGAGCAGTTCTTC |
| 2958 | 96-TL684-TIL-CD8+_CD103− | TIL | CD8+_CD103− | TGTGCCAGCAGCTTAGGATCAGGGGTTGAAGCTTTCTTT |
| 2959 | 96-TL684-TIL-CD8+_CD103− | TIL | CD8+_CD103− | TGTGCCAGCAGCCAGAGACTAGCAGACTACAATGAGCAGTTCTTC |
| 2960 | 96-TL684-TIL-CD8+_CD103− | TIL | CD8+_CD103− | TGCAGTGCTAGAGATGGTACTAGCGGGGCCCCCTATGAGCAGTTCTTC |
| 2961 | 96-TL684-TIL-CD8+_CD103− | TIL | CD8+_CD103− | TGTGCCAGCACCCCTGCGGGGTTCCATAATGAAAAGCTGTTTTTT |
| 2962 | 96-TL684-TIL-CD8+_CD103− | TIL | CD8+_CD103− | TGTGCCAGCAGTGAAGACAGGGCCGGCCAAGAGACCCAGTACTTC |
| 2963 | 96-TL684-TIL-CD8+_CD103− | TIL | CD8+_CD103− | TGTGCCAGCAGCTTAGCCTCGGGCGGGGACTCCCAAGAGACCCAGTACTTC |
| 2964 | 96-TL684-TIL-CD8+_CD103− | TIL | CD8+_CD103− | TGCAGTGCTAGAGATGCCGGGGGATACTATGGCTACACCTTC |
| 2965 | 96-TL684-TIL-CD8+_CD103− | TIL | CD8+_CD103− | TGTGCCTGCTCCACGGGCAGGACGCAAGAGACCCAGTACTTC |
| 2966 | 96-TL684-TIL-CD8+_CD103− | TIL | CD8+_CD103− | TGTGCCAGCAGTTTTGGCTACGAGCAGTACTTC |
| 2967 | 96-TL684-TIL-CD8+_CD103− | TIL | CD8+_CD103− | TGCAGCGTTGGGGACAGGGGACTAAAAGATACGCAGTATTTT |
| 2968 | 96-TL684-TIL-CD8+_CD103− | TIL | CD8+_CD103− | TGTGCCAGCAGCCCAGGGAGTGGGGCTGGCTATGGCTACACCTTC |
| 2969 | 96-TL684-TIL-CD8+_CD103− | TIL | CD8+_CD103− | TGTGCCAGCAGCTTGGGGCTAGCGGGGCGCCCCTCTGAGCAGTTCTTC |
| 2970 | 96-TL684-TIL-CD8+_CD103− | TIL | CD8+_CD103− | TGTGCCACCAGTGATGCCGCAACAGGGCGGTGGACCGGGGAGCTGTTTTTT |
| 2971 | 96-TL684-TIL-CD8+_CD103− | TIL | CD8+_CD103− | TGTGCCAGCAGTTTACATACTGGACTTACCTCCGAAGAGCAGTACTTC |
| 2972 | 96-TL684-TIL-CD8+_CD103− | TIL | CD8+_CD103− | TGCAGTGCTAGAGATACAGGACTAGAATACAATGAGCAGTTCTTC |
| 2973 | 96-TL684-TIL-CD8+_CD103− | TIL | CD8+_CD103− | TGCGCCAGCAGCCAAGATTCGGGCTCTGGGGCCAACGTCCTGACTTTC |
| 2974 | 96-TL684-TIL-CD8+_CD103− | TIL | CD8+_CD103− | TGTAGGGGGGTTGGGCCGGGGGAGCAGTACTTC |
| 2975 | 96-TL684-TIL-CD8+_CD103− | TIL | CD8+_CD103− | TGTGCCAGCAGCTCCCGGGACAGGGCCTACGAGCAGTACTTC |

TABLE 7
Specifically enriched genes in each 10x cluster

| | padj. value. | padj. value. | padj. val ue. | padj. value. | Min. | padj_Min. |
|---|---|---|---|---|---|---|
| | vs. V | vs. IV | vs. III | vs. II | log2FC | log2FC |
| FOS | NA | 1.66E−82 | 6.63E−42 | 5.90E−54 | 2.620190055 | 6.63E−42 |
| TNFAIP3 | NA | 1.58E−77 | 1.79E−131 | 7.53E−21 | 2.265213659 | 7.53E−21 |
| JUNB | NA | 1.68E−61 | 1.26E−30 | 4.72E−23 | 2.21557016 | 1.26E−30 |
| ZFP36 | NA | 7.43E−53 | 2.03E−91 | 3.48E−29 | 2.918441617 | 3.48E−29 |
| FOSB | NA | 2.20E−50 | 5.67E−17 | 2.48E−29 | 1.769295602 | 5.67E−17 |
| TSC22D3 | NA | 8.38E−58 | 3.21E−132 | 1.27E−13 | 1.269885151 | 1.27E−13 |
| KLF6 | NA | 7.95E−52 | 6.29E−50 | 1.66E−10 | 1.350296963 | 1.66E−10 |
| IL7R | NA | 1.63E−47 | 1.84E−43 | 1.50E−82 | 2.602331444 | 1.84E−43 |
| ANXA1 | NA | 7.69E−32 | 6.33E−21 | 8.62E−45 | 2.064086901 | 6.33E−21 |
| RGCC | NA | 1.04E−45 | 1.06E−42 | 0.000401616 | 1.12087222 | 0.000401616 |
| VIM | NA | 1.42E−24 | 4.76E−46 | 1.08E−29 | 2.631230111 | 1.42E−24 |
| NFKBIA | NA | 2.17E−37 | 6.30E−24 | 2.97E−14 | 1.996504035 | 6.30E−24 |
| GPR183 | NA | 1.81E−45 | 3.88E−52 | 3.00E−59 | 2.419298421 | 3.88E−52 |
| FTH1 | NA | 7.43E−53 | 9.06E−85 | 1.81E−43 | 2.200051083 | 1.81E−43 |
| ZFP36L2 | NA | 1.14E−22 | 1.90E−88 | 2.06E−21 | 1.884610725 | 2.06E−21 |
| CD69 | NA | 1.51E−35 | 1.49E−56 | 2.18E−07 | 1.035505091 | 2.18E−07 |
| IER2 | NA | 8.74E−21 | 0.002093792 | 7.85E−16 | 0.857291392 | 0.002093792 |
| CXCR4 | NA | 2.28E−26 | 1.00E−120 | 5.50E−10 | 1.193942163 | 5.50E−10 |
| REL | NA | 2.13E−21 | 3.31E−19 | 2.91E−09 | 1.369002869 | 2.91E−09 |
| RORA | NA | 3.93E−17 | 5.06E−36 | 7.78E−13 | 1.87840742 | 3.93E−17 |
| BTG2 | NA | 7.14E−22 | 0.000122431 | 6.57E−11 | 0.873222337 | 0.000122431 |
| MYADM | NA | 7.69E−32 | 2.44E−27 | 4.11E−24 | 1.578811291 | 2.44E−27 |
| ANKRD28 | NA | 1.16E−15 | 2.03E−19 | 7.49E−05 | 0.263208789 | 7.49E−05 |
| MCL1 | NA | 1.50E−13 | 4.28E−26 | 2.30E−05 | 1.238637333 | 2.30E−05 |
| PNRC1 | NA | 2.82E−10 | 1.70E−17 | 0.003629103 | 0.945575052 | 0.003629103 |
| TUBA1A | NA | 3.17E−17 | 9.60E−08 | 4.14E−05 | 0.887158592 | 4.14E−05 |
| CD44 | NA | 6.64E−11 | 3.37E−27 | 9.28E−09 | 1.629746169 | 6.64E−11 |
| YPEL5 | NA | 1.92E−15 | 2.53E−22 | 1.76E−06 | 1.224033473 | 1.76E−06 |
| PTGER4 | NA | 1.50E−13 | 2.38E−22 | 6.44E−05 | 1.204635078 | 6.44E−05 |
| IFITM2 | NA | 1.09E−09 | 2.02E−05 | 2.34E−10 | 1.08193307 | 2.02E−05 |
| SELK | NA | 3.40E−12 | 9.41E−13 | 0.005753149 | 0.727586968 | 0.005753149 |
| S100A10 | NA | 2.56E−09 | 1.78E−16 | 2.25E−57 | 1.521791897 | 2.56E−09 |
| PABPC1 | NA | 1.87E−47 | 1.78E−30 | 5.02E−27 | 0.884586386 | 1.78E−30 |
| LMNA | NA | 1.12E−30 | 1.57E−40 | 1.29E−28 | 1.437010868 | 1.12E−30 |
| LEPROTL1 | NA | 3.23E−10 | 1.68E−10 | 5.28E−06 | 1.061103392 | 5.28E−06 |
| SAT1 | NA | 4.69E−07 | 1.98E−05 | 1.11E−15 | 0.095828191 | 1.98E−05 |
| TOBI | NA | 3.73E−12 | 5.49E−21 | 1.45E−15 | 1.307119171 | 3.73E−12 |
| FUS | NA | 1.06E−08 | 4.19E−06 | 1.89E−05 | 0.327001458 | 1.89E−05 |
| ODF2L | NA | 6.35E−09 | 1.12E−08 | 2.90E−05 | 1.009673232 | 2.90E−05 |
| IDS | NA | 3.82E−08 | 8.49E−11 | 1.21E−05 | 1.139317416 | 1.21E−05 |
| DDX3X | NA | 8.31E−09 | 4.48E−07 | 0.000894747 | 0.474753873 | 0.000894747 |
| SYTL3 | NA | 1.84E−06 | 5.40E−17 | 0.000253146 | 0.747116369 | 0.000253146 |
| RP11-138A9 | NA | 5.39E−08 | 2.42E−06 | 9.96E−09 | 0.863328487 | 2.42E−06 |
| FAM46C | NA | 5.06E−13 | 2.80E−34 | 3.51E−05 | 0.847977473 | 3.51E−05 |
| CRIP1 | NA | 0.001624171 | 2.75E−10 | 0.009834552 | 0.94600557 | 0.009834552 |
| ELF1 | NA | 0.000138316 | 2.05E−07 | 2.46E−05 | 0.598563385 | 2.46E−05 |
| AHNAK | NA | 6.01E−08 | 6.41E−16 | 2.73E−11 | 1.007746639 | 6.01E−08 |
| HIST1H4C | NA | 1.49E−06 | 7.72E−07 | 3.78E−08 | 0.930026463 | 7.72E−07 |
| SDCBP | NA | 4.06E−08 | 1.41E−06 | 0.000141025 | 0.257116448 | 0.000141025 |
| RPLP0 | NA | 0.008779115 | 0.003171103 | 1.72E−11 | 0.882868721 | 0.003171103 |
| ARL4A | NA | 6.33E−13 | 3.74E−12 | 2.22E−08 | 0.84659306 | 3.74E−12 |
| GPR65 | NA | 0.000107073 | 3.79E−11 | 0.001590716 | 0.701424179 | 0.001590716 |
| H3F3B | NA | 4.72E−10 | 1.53E−16 | 2.42E−09 | 0.826890053 | 2.42E−09 |
| IRF1 | NA | 1.23E−06 | 0.000197943 | 2.99E−11 | 0.665502687 | 0.000197943 |
| RSL24D1 | NA | 6.36E−06 | 4.22E−06 | 0.003507975 | 0.570895999 | 0.003507975 |
| EIF1 | NA | 1.02E−17 | 2.59E−48 | 0.002079569 | 0.347194993 | 0.002079569 |
| JMJD1C | NA | 2.15E−06 | 0.000384594 | 0.001770106 | 0.69361394 | 0.000384594 |
| VAMP2 | NA | 1.04E−05 | 4.00E−11 | 4.52E−06 | 0.845807406 | 4.52E−06 |
| YWHAZ | NA | 0.001437739 | 2.04E−10 | 0.000145732 | 0.917970678 | 0.001437739 |
| HOPX | NA | 0.000188042 | 8.77E−10 | 6.66E−27 | 0.91334475 | 0.000188042 |
| FOSL2 | NA | 2.65E−10 | 7.72E−18 | 4.66E−08 | 0.749791815 | 4.66E−08 |
| PIK3R1 | NA | 0.003094919 | 1.94E−12 | 4.00E−33 | 0.891986772 | 0.003094919 |
| EML4 | NA | 0.002137792 | 1.53E−23 | 3.09E−10 | 0.842854321 | 0.002137792 |
| BTG1 | NA | 7.69E−32 | 8.31E−60 | 5.26E−21 | 0.705782569 | 5.26E−21 |
| CD55 | NA | 2.03E−08 | 1.07E−10 | 3.39E−13 | 0.814799096 | 2.03E−08 |
| YBX3 | NA | 2.26E−09 | 1.19E−11 | 1.70E−13 | 0.806710572 | 1.19E−11 |
| PDE4B | NA | 3.77E−06 | 9.92E−08 | 1.43E−05 | 0.806626131 | 3.77E−06 |
| NFE2L2 | NA | 1.53E−06 | 5.49E−09 | 1.66E−06 | 0.753039458 | 1.66E−06 |
| KLRC1 | NA | 1.26E−05 | 4.75E−12 | 5.03E−13 | 0.800761769 | 1.26E−05 |
| RNF125 | NA | 2.54E−08 | 2.56E−15 | 0.002012095 | 0.634122788 | 0.002012095 |
| RPS20 | NA | 6.54E−05 | 6.17E−15 | 8.67E−26 | 0.778950288 | 6.54E−05 |
| MT-ND1 | NA | 0.007093368 | 3.72E−08 | 2.58E−05 | 0.775046717 | 0.007093368 |
| LYAR | NA | 1.24E−05 | 7.84E−05 | 3.42E−31 | 0.674717264 | 7.84E−05 |
| AC016831.7 | NA | 1.68E−08 | 3.03E−12 | 6.50E−06 | 0.769830524 | 1.68E−08 |
| CCNL1 | NA | 0.004250662 | 0.000149318 | 0.000568016 | 0.663748163 | 0.000568016 |
| RPS5 | NA | 0.003560905 | 6.62E−08 | 1.93E−12 | 0.751571098 | 0.003560905 |
| SVIP | NA | 0.001454861 | 0.002421377 | 7.73E−05 | 0.638646302 | 0.002421377 |
| RPS3A | NA | 4.69E−07 | 9.96E−18 | 3.51E−18 | 0.7292654 | 4.69E−07 |
| RPL9 | NA | 3.11E−05 | 1.61E−14 | 1.60E−21 | 0.713109274 | 3.11E−05 |
| PDCD4 | NA | 0.007227659 | 0.001269895 | 3.29E−08 | 0.696493816 | 0.007227659 |
| FAM177A1 | NA | 0.001073118 | 0.000765204 | 1.63E−05 | 0.65792054 | 0.000765204 |
| SBDS | NA | 3.73E−05 | 2.70E−07 | 2.70E−06 | 0.675014647 | 3.73E−05 |
| SNRPG | NA | 5.61E−05 | 0.007121432 | 5.02E−06 | 0.288220813 | 5.02E−06 |
| TUBA1B | NA | 0.005875971 | 0.00159609 | 0.005870285 | 0.56870654 | 0.005870285 |
| VPS37B | NA | 2.90E−06 | 6.37E−17 | 0.002169337 | 0.466707881 | 0.002169337 |
| CDC42SE2 | NA | 0.000318843 | 0.009794205 | 9.76E−07 | 0.426037826 | 0.009794205 |
| RPL17 | NA | 0.000630659 | 0.001422255 | 1.12E−08 | 0.536793345 | 0.001422255 |
| PARP8 | NA | 0.000786244 | 6.64E−06 | 6.32E−08 | 0.630954581 | 0.000786244 |
| STAT4 | NA | 0.002183591 | 1.07E−12 | 6.36E−06 | 0.629984817 | 0.002183591 |
| TAGLN2 | NA | 0.008068302 | 2.55E−07 | 4.11E−10 | 0.61816233 | 0.008068302 |
| KDM6B | NA | 7.63E−09 | 9.02E−07 | 0.003503394 | 0.469156481 | 0.003503394 |
| CSRNP1 | NA | 1.15E−08 | 3.86E−07 | 0.006290294 | 0.347032927 | 0.006290294 |
| SERPINB9 | NA | 6.80E−05 | 0.001015828 | 0.005581702 | 0.265611197 | 0.005581702 |
| MT-ND2 | NA | 6.27E−05 | 2.78E−22 | 1.77E−11 | 0.561392112 | 6.27E−05 |
| MT-CO3 | NA | 0.001084082 | 7.15E−23 | 7.60E−05 | 0.470003688 | 7.60E−05 |
| RPL14 | NA | 1.33E−05 | 9.62E−32 | 1.57E−29 | 0.541894813 | 1.33E−05 |
| HNRNPUL1 | NA | 0.001475459 | 0.000513418 | 0.00070165 | 0.531020853 | 0.000513418 |
| SKIL | NA | 0.002695606 | 2.83E−06 | 4.92E−07 | 0.535590273 | 0.002695606 |
| EIF1AX | NA | 0.002892936 | 0.001131648 | 2.73E−05 | 0.522532099 | 0.002892936 |
| PERP | NA | 0.00018877 | 7.16E−08 | 9.54E−16 | 0.498498324 | 0.00018877 |
| DNAJB9 | NA | 3.01E−05 | 1.43E−07 | 0.000128016 | 0.489115584 | 0.000128016 |
| TSC22D2 | NA | 1.16E−06 | 0.005292869 | 0.000113539 | 0.33233768 | 0.005292869 |
| CCND3 | NA | 0.001133665 | 0.000122431 | 2.23E−05 | 0.488870983 | 0.001133665 |
| FRMD4B | NA | 0.000908681 | 0.000501564 | 9.85E−09 | 0.445221505 | 0.000501564 |
| RP11-138A9 | NA | 0.005651856 | 2.16E−07 | 8.35E−05 | 0.474233683 | 0.005651856 |
| ATP2B1 | NA | 0.001485717 | 1.11E−06 | 3.36E−07 | 0.46857568 | 0.001485717 |
| RTN4 | NA | 0.000805944 | 0.003453303 | 7.79E−05 | 0.375509687 | 7.79E−05 |
| CD48 | NA | 0.009150862 | 0.000664974 | 7.86E−07 | 0.418732891 | 0.009150862 |
| DSTN | NA | 0.002904998 | 0.000186724 | 1.06E−15 | 0.417567773 | 0.002904998 |
| PSME4 | NA | 0.008174178 | 0.001185436 | 0.006823469 | 0.387246929 | 0.006823469 |
| FXYD2 | NA | 1.92E−07 | 8.51E−08 | 3.74E−17 | 0.404561207 | 8.51E−08 |
| RPS4X | NA | 0.000924117 | 1.20E−10 | 3.85E−28 | 0.381946846 | 0.000924117 |
| MAP3K8 | NA | 0.000175794 | 6.51E−05 | 0.000127269 | 0.216967299 | 0.000127269 |
| AUTS2 | NA | 0.005686021 | 4.23E−06 | 2.46E−09 | 0.371972221 | 0.005686021 |
| RPL3 | NA | 0.001912992 | 1.99E−08 | 1.11E−24 | 0.361285551 | 0.001912992 |
| MYBL1 | NA | 0.004982183 | 1.64E−06 | 6.07E−17 | 0.353506755 | 0.004982183 |
| MIR29A | NA | 2.01E−06 | 1.49E−05 | 0.00070165 | 0.312081318 | 1.49E−05 |
| RPS8 | NA | 0.005759353 | 7.12E−15 | 2.10E−26 | 0.321078519 | 0.005759353 |
| PTMA | NA | 6.68E−12 | 3.30E−31 | 3.15E−06 | 0.173349586 | 3.15E−06 |
| EEF1A1 | NA | 0.001809716 | 1.53E−25 | 2.68E−37 | 0.28930546 | 0.001809716 |
| RPS12 | NA | 6.76E−08 | 2.50E−24 | 4.85E−60 | 0.258381871 | 6.76E−08 |
| CLU | NA | 0.002664342 | 9.28E−05 | 3.65E−12 | 0.258347767 | 0.002664342 |
| RPS14 | NA | 5.25E−05 | 1.75E−14 | 3.61E−47 | 0.239824473 | 5.25E−05 |
| RPL32 | NA | 3.82E−05 | 3.88E−13 | 3.93E−40 | 0.214370857 | 3.82E−05 |
| RPS25 | NA | 0.000648948 | 1.87E−11 | 2.53E−26 | 0.206461064 | 0.000648948 |
| RPL39 | NA | 0.000565095 | 1.74E−16 | 2.80E−43 | 0.195344507 | 0.000565095 |
| RPL34 | NA | 0.000820145 | 7.92E−18 | 2.10E−32 | 0.18751242 | 0.000820145 |
| RPL10 | NA | 0.000501243 | 1.98E−14 | 7.53E−35 | 0.184865229 | 0.000501243 |
| EMB | NA | 0.004669363 | 0.000271315 | 9.68E−05 | 0.165589987 | 0.004669363 |
| RPLP2 | NA | 0.009961665 | 1.87E−26 | 3.62E−61 | 0.154274807 | 0.009961665 |
| RPS29 | NA | 0.000799702 | 1.51E−28 | 3.69E−59 | 0.143164341 | 0.000799702 |
| RAP1B | NA | 0.006692062 | 0.002021216 | 2.97E−12 | 0.066991093 | 0.006692062 |
| CREM | 0.000637878 | 2.23E−13 | 7.25E−17 | NA | 0.16411119 | 0.000637878 |
| SAMSN1 | 4.40E−06 | 3.48E−07 | 3.53E−13 | NA | 0.249849391 | 4.40E−06 |
| SRGN | 1.31E−06 | 4.57E−13 | 6.71E−21 | NA | 0.037430009 | 1.31E−06 |
| SRSF5 | 1.90E−07 | 1.46E−08 | 3.95E−12 | NA | 0.035995217 | 1.90E−07 |
| BIRC3 | 0.000244594 | 3.20E−09 | 0.002259756 | NA | 0.410890215 | 0.000244594 |
| PHLDA1 | 9.04E−14 | 1.41E−29 | 3.04E−21 | NA | 1.460838105 | 9.04E−14 |
| STAT3 | 2.48E−14 | 2.67E−12 | 4.05E−09 | NA | 1.210310952 | 2.48E−14 |
| ETS1 | 3.31E−19 | 0.001454482 | 2.00E−15 | NA | 1.228327744 | 3.31E−19 |
| ETV1 | 3.83E−21 | 9.49E−05 | 0.001411419 | NA | 0.477436057 | 0.001411419 |
| NEAT1 | 2.24E−25 | 7.06E−13 | 2.70E−05 | NA | 1.108735317 | 2.70E−05 |
| KRT86 | 5.53E−60 | 1.68E−26 | 3.58E−28 | NA | 1.991594638 | 3.58E−28 |
| AKAP5 | 2.08E−34 | 8.64E−11 | 0.000495119 | NA | 0.737927495 | 0.000495119 |
| HLA-DQA1 | 4.38E−33 | 1.38E−06 | 1.21E−07 | NA | 0.973184844 | 1.21E−07 |
| CXCL13 | 1.21E−146 | 2.32E−69 | 7.62E−40 | NA | 3.126576592 | 7.62E−40 |
| CHN1 | 7.22E−65 | 3.28E−21 | 2.81E−27 | NA | 1.8586028 | 3.28E−21 |
| TNIP3 | 2.53E−28 | 1.10E−08 | 3.42E−05 | NA | 0.96911344 | 3.42E−05 |
| TIGIT | 4.29E−61 | 1.12E−22 | 8.59E−14 | NA | 1.790082066 | 8.59E−14 |
| LYST | 1.57E−24 | 1.95E−05 | 1.25E−08 | NA | 1.197928623 | 1.95E−05 |
| CTSW | 5.45E−23 | 0.000690254 | 3.98E−07 | NA | 1.430011984 | 3.98E−07 |
| HAVCR2 | 6.29E−78 | 2.02E−26 | 4.86E−12 | NA | 1.56926631 | 4.86E−12 |
| AMICA1 | 2.10E−32 | 2.93E−06 | 1.15E−12 | NA | 1.557097802 | 2.93E−06 |
| ALOX5AP | 1.73E−26 | 0.003772 | 8.85E−11 | NA | 1.181535785 | 0.003772 |
| AC002331.1 | 2.87E−63 | 3.61E−24 | 6.17E−11 | NA | 1.763215521 | 6.17E−11 |
| AC092580.4 | 5.63E−85 | 1.60E−19 | 1.44E−29 | NA | 1.490695634 | 1.60E−19 |
| SIRPG | 4.37E−37 | 0.000329935 | 2.45E−05 | NA | 0.960381084 | 2.45E−05 |
| HLA-DRA | 2.43E−50 | 2.04E−11 | 1.27E−10 | NA | 1.700525516 | 1.27E−10 |
| CD74 | 7.78E−36 | 2.60E−05 | 2.48E−06 | NA | 1.493444518 | 2.48E−06 |
| SRGAP3 | 1.82E−43 | 6.23E−11 | 4.12E−08 | NA | 1.377296108 | 4.12E−08 |
| HLA-DPA1 | 1.73E−29 | 0.003946769 | 5.07E−05 | NA | 1.105636301 | 0.003946769 |
| AC069363.1 | 4.52E−43 | 3.02E−08 | 1.52E−05 | NA | 1.0532031 | 1.52E−05 |
| RBPJ | 5.99E−97 | 2.06E−33 | 6.86E−37 | NA | 3.430066413 | 6.86E−37 |
| NKG7 | 7.63E−23 | 0.00490394 | 0.003416563 | NA | 0.615222066 | 0.00490394 |
| HLA-DPB1 | 2.31E−33 | 0.00399397 | 2.47E−07 | NA | 1.17285258 | 0.00399397 |
| ENTPD1 | 7.75E−118 | 1.44E−25 | 5.10E−18 | NA | 2.237692196 | 5.10E−18 |
| HLA-DRB1 | 1.08E−55 | 1.10E−09 | 9.61E−10 | NA | 1.867004057 | 9.61E−10 |
| GZMA | 1.09E−42 | 0.003619451 | 2.76E−10 | NA | 0.717131097 | 0.003619451 |
| RGS1 | 5.90E−76 | 1.22E−46 | 2.72E−103 | NA | 2.731407573 | 5.90E−76 |
| RP11-347P5. | 0.000420226 | 0.001942401 | 3.51E−07 | NA | 0.2373155 | 0.000420226 |
| CLEC2B | 0.000244272 | 3.65E−05 | 1.16E−11 | NA | 0.557073889 | 0.000244272 |
| RNF19A | 6.01E−20 | 1.56E−16 | 1.25E−26 | NA | 1.534939563 | 6.01E−20 |
| KRT81 | 5.98E−13 | 8.34E−05 | 0.003052277 | NA | 0.376158507 | 0.003052277 |
| RP11-279F6. | 9.44E−16 | 0.004695777 | 0.002043077 | NA | 0.353215743 | 0.004695777 |
| TNS3 | 1.13E−14 | 1.48E−09 | 0.003798297 | NA | 0.347046461 | 0.003798297 |
| MAST4 | 1.69E−14 | 0.000994731 | 0.000913076 | NA | 0.599970917 | 0.000913076 |
| LAYN | 2.17E−35 | 1.42E−17 | 9.39E−10 | NA | 0.858923999 | 9.39E−10 |
| TNFRSF18 | 1.34E−22 | 2.61E−12 | 2.91E−05 | NA | 0.651260673 | 2.91E−05 |
| VCAM1 | 3.43E−31 | 3.78E−15 | 1.36E−07 | NA | 0.892616431 | 1.36E−07 |
| AHI1 | 6.00E−25 | 1.59E−09 | 4.53E−05 | NA | 0.809032683 | 4.53E−05 |
| ACP5 | 5.05E−18 | 3.58E−05 | 0.00381038 | NA | 0.699716551 | 0.00381038 |
| TNFRSF9 | 8.16E−53 | 1.44E−25 | 2.38E−09 | NA | 1.010907938 | 2.38E−09 |
| RAB27A | 9.38E−19 | 0.000457807 | 0.009467496 | NA | 0.863443141 | 0.009467496 |
| SLA | 7.06E−20 | 0.000223607 | 0.007230644 | NA | 1.008695747 | 0.007230644 |
| ITGAE | 1.44E−23 | 8.44E−09 | 1.16E−06 | NA | 1.404487179 | 1.16E−06 |
| CRTAM | 1.72E−34 | 1.03E−12 | 5.22E−08 | NA | 1.273678799 | 5.22E−08 |
| CTLA4 | 2.95E−108 | 5.96E−57 | 8.96E−54 | NA | 3.634958175 | 8.96E−54 |
| CCL3 | 3.08E−42 | 6.42E−22 | 1.83E−14 | NA | 2.186450539 | 1.83E−14 |
| IFNG | 3.98E−40 | 1.24E−19 | 6.99E−09 | NA | 1.926260214 | 6.99E−09 |
| CYSLTR1 | 0.003483664 | 0.00846443 | 0.000542256 | NA | 0.26981673 | 0.003483664 |
| HLA-A | 7.70E−07 | 0.000575743 | 2.83E−16 | NA | 0.222076438 | 0.000575743 |
| RGS13 | 4.29E−09 | 1.12E−07 | 4.12E−08 | NA | 0.345333169 | 4.12E−08 |
| IL26 | 2.58E−06 | 3.16E−05 | 1.41E−05 | NA | 0.335981266 | 1.41E−05 |
| IL17A | 4.68E−09 | 4.38E−06 | 2.96E−07 | NA | 0.387444572 | 2.96E−07 |
| MYO1E | 1.12E−08 | 0.000787688 | 3.80E−09 | NA | 0.356887185 | 0.000787688 |
| TNFSF4 | 5.04E−09 | 1.30E−05 | 0.000906747 | NA | 0.310849396 | 0.000906747 |
| AFAP1L2 | 1.97E−13 | 4.89E−06 | 3.55E−05 | NA | 0.40411992 | 3.55E−05 |
| AGFG1 | 1.32E−06 | 0.000172958 | 0.000146563 | NA | 0.425946297 | 0.000146563 |
| CSGALNACT1 | 2.21E−09 | 0.001466406 | 0.000268602 | NA | 0.385809284 | 0.000268602 |
| CBLB | 3.42E−05 | 0.005678513 | 0.003085899 | NA | 0.581796519 | 3.42E−05 |
| PDCD1 | 2.72E−06 | 0.003085605 | 0.008364075 | NA | 0.526289264 | 0.008364075 |
| CLECL1 | 5.40E−08 | 2.52E−05 | 1.39E−09 | NA | 0.762149336 | 2.52E−05 |
| ARID5B | 4.13E−07 | 3.93E−06 | 1.48E−06 | NA | 0.794915971 | 4.13E−07 |
| ARL3 | 1.65E−13 | 0.002114441 | 0.005412229 | NA | 0.509782342 | 0.005412229 |
| SNX9 | 2.86E−11 | 1.39E−08 | 4.92E−09 | NA | 0.768488877 | 4.92E−09 |
| NR3C1 | 1.50E−06 | 5.68E−06 | 3.96E−07 | NA | 0.874460583 | 1.50E−06 |
| PRDM1 | 5.23E−12 | 0.00266954 | 1.20E−06 | NA | 0.88275225 | 5.23E−12 |
| ICOS | 3.02E−06 | 1.22E−09 | 3.50E−11 | NA | 0.88604441 | 3.02E−06 |
| MIR155HG | 1.22E−14 | 3.12E−05 | 1.48E−06 | NA | 0.693045083 | 1.48E−06 |
| CD7 | 1.79E−10 | 0.003289914 | 2.94E−06 | NA | 0.916916051 | 1.79E−10 |
| PTPN22 | 2.33E−09 | 0.001275942 | 3.66E−10 | NA | 0.968242771 | 2.33E−09 |
| CALR | 5.60E−08 | 0.009999931 | 5.51E−05 | NA | 0.982686939 | 0.009999931 |
| ID2 | 6.42E−16 | 0.000170191 | 3.77E−05 | NA | 0.875841563 | 3.77E−05 |
| PRF1 | 1.75E−07 | 1.50E−05 | 0.002263138 | NA | 1.048524967 | 0.002263138 |
| TOX | 3.13E−20 | 6.37E−05 | 9.42E−11 | NA | 0.769197704 | 6.37E−05 |
| GZMB | 1.34E−06 | 3.51E−10 | 5.32E−08 | NA | 1.221935285 | 1.34E−06 |
| ZEB2 | 1.86E−10 | 9.42E−11 | 9.00E−15 | NA | 1.249618159 | 1.86E−10 |
| PAG1 | 7.77E−15 | 1.26E−06 | 2.52E−05 | NA | 1.040188511 | 2.52E−05 |
| KLRD1 | 1.12E−14 | 0.000204255 | 5.79E−06 | NA | 1.221275836 | 5.79E−06 |
| CLEC2D | 1.84E−16 | 1.74E−07 | 1.74E−12 | NA | 1.425987745 | 1.84E−16 |
| HLA-DRB5 | 1.53E−27 | 1.74E−06 | 2.66E−15 | NA | 1.098554982 | 1.74E−06 |
| ITM2A | 2.55E−25 | 1.24E−09 | 6.71E−10 | NA | 1.681694597 | 6.71E−10 |
| DUSP4 | 7.60E−63 | 2.03E−45 | 1.70E−46 | NA | 3.575838252 | 1.70E−46 |
| PPP1R15A | 2.41E−11 | 3.42E−18 | NA | 2.24E−11 | 0.464034832 | 2.41E−11 |
| JUN | 1.87E−26 | 3.15E−65 | NA | 9.37E−16 | 1.180205182 | 1.87E−26 |
| DNAJA1 | 1.69E−36 | 1.94E−44 | NA | 8.68E−31 | 1.876239108 | 1.69E−36 |
| GADD45B | 0.0001058 | 1.82E−07 | NA | 2.73E−21 | 0.43259821 | 0.0001058 |
| HSP90AB1 | 2.58E−56 | 1.36E−40 | NA | 4.55E−37 | 1.994368885 | 2.58E−56 |
| NEU1 | 9.13E−08 | 1.02E−21 | NA | 2.18E−10 | 0.769179396 | 9.13E−08 |
| HSPA6 | 8.48E−145 | 5.35E−76 | NA | 8.16E−76 | 3.408403253 | 5.35E−76 |
| AC006129.2 | 7.48E−27 | 6.94E−07 | NA | 0.000474985 | 0.931576889 | 0.000474985 |
| HSP90AA1 | 4.56E−228 | 9.78E−136 | NA | 7.31E−122 | 2.867834259 | 9.78E−136 |
| HSPE1 | 1.66E−152 | 2.75E−77 | NA | 5.06E−70 | 3.80539206 | 2.75E−77 |
| HSPB1 | 1.44E−204 | 8.65E−101 | NA | 8.71E−71 | 4.39192139 | 8.65E−101 |
| HSPA1B | 0 | 1.59E−166 | NA | 3.66E−167 | 5.75800638 | 1.59E−166 |
| HSPA1A | 0 | 5.20E−156 | NA | 3.71E−173 | 5.638622319 | 5.20E−156 |
| MRPL18 | 1.29E−05 | 7.33E−05 | NA | 7.65E−06 | 0.464014474 | 1.29E−05 |
| HIST2H2AA3 | 0.00327089 | 3.93E−09 | NA | 4.40E−06 | 0.475956969 | 0.00327089 |
| C17orf67 | 6.60E−10 | 0.00308263 | NA | 0.001284567 | 0.380442359 | 0.00308263 |
| GPR113 | 8.30E−17 | 3.89E−08 | NA | 1.22E−07 | 0.499732558 | 3.89E−08 |
| TRA2B | 1.17E−05 | 6.18E−06 | NA | 1.54E−07 | 0.606306382 | 1.17E−05 |
| TCP1 | 6.70E−08 | 0.002311184 | NA | 0.000213795 | 0.530155811 | 0.002311184 |
| HSD17B7 | 2.72E−13 | 0.000222937 | NA | 0.00019165 | 0.461103562 | 0.000222937 |
| NUDT4 | 1.26E−05 | 1.06E−06 | NA | 8.03E−06 | 0.769380579 | 1.26E−05 |
| NR4A1 | 2.03E−10 | 2.32E−16 | NA | 5.32E−08 | 0.865822254 | 2.03E−10 |
| DNAJA4 | 1.11E−25 | 4.91E−15 | NA | 9.33E−12 | 0.803134197 | 4.91E−15 |
| MB21D1 | 1.14E−18 | 1.31E−05 | NA | 5.04E−12 | 0.650130282 | 1.31E−05 |
| SERPIN H1 | 1.20E−28 | 6.61E−15 | NA | 5.04E−12 | 0.829860665 | 6.61E−15 |
| DONSON | 1.03E−14 | 0.002853788 | NA | 2.71E−15 | 0.736069594 | 0.002853788 |
| ZFAND2A | 4.16E−20 | 6.69E−16 | NA | 4.41E−12 | 1.066658182 | 4.16E−20 |
| TSPYL2 | 1.28E−08 | 3.62E−16 | NA | 1.85E−05 | 1.080709987 | 1.28E−08 |
| UGP2 | 1.71E−15 | 5.63E−06 | NA | 0.000844439 | 0.884934197 | 5.63E−06 |
| MXD1 | 5.46E−20 | 1.92E−15 | NA | 2.65E−09 | 1.069026257 | 2.65E−09 |
| FTL | 1.02E−28 | 1.66E−13 | NA | 6.54E−22 | 1.169331909 | 1.02E−28 |
| UBB | 1.63E−29 | 1.32E−20 | NA | 1.47E−10 | 1.24307025 | 1.63E−29 |
| BAG3 | 1.73E−45 | 2.85E−32 | NA | 4.82E−18 | 1.224624597 | 4.82E−18 |
| CHORDC1 | 2.08E−27 | 7.44E−17 | NA | 1.92E−09 | 1.268567875 | 1.92E−09 |
| UBC | 8.93E−39 | 5.84E−34 | NA | 1.41E−05 | 0.902441099 | 1.41E−05 |
| DNAJB4 | 5.52E−56 | 1.09E−42 | NA | 6.21E−28 | 2.038178703 | 1.09E−42 |
| CACYBP | 1.19E−52 | 2.35E−34 | NA | 1.53E−29 | 2.116008605 | 2.35E−34 |
| HSPH1 | 7.63E−84 | 3.15E−65 | NA | 8.68E−50 | 2.988971735 | 3.15E−65 |
| HSPA8 | 3.87E−75 | 2.06E−53 | NA | 1.54E−52 | 3.185712724 | 2.06E−53 |
| HSPD1 | 5.83E−89 | 6.15E−53 | NA | 1.72E−65 | 2.942737466 | 6.15E−53 |
| RGS2 | 1.16E−83 | 1.09E−61 | NA | 0.003360863 | 1.196385902 | 0.003360863 |
| DNAJB1 | 1.15E−221 | 1.35E−148 | NA | 2.53E−160 | 4.824072522 | 1.35E−148 |
| CD52 | 2.69E−08 | NA | 3.20E−06 | 5.80E−05 | 0.484125919 | 2.69E−08 |
| ATP5E | 1.03E−12 | NA | 0.000456224 | 0.00057577 | 0.433144665 | 0.000456224 |
| IL32 | 6.33E−19 | NA | 9.55E−06 | 4.69E−05 | 0.480236359 | 9.55E−06 |

### REFERENCES

1. Galon, J. et al. Type, density, and location of immune cells within human colorectal tumors predict clinical outcome. Science 313, 1960-4 (2006).
2. Wherry, E. J. T cell exhaustion. Nat. Immunol. 12, 492-499 (2011).
3. Pardoll, D. M. The blockade of immune checkpoints in cancer immunotherapy. Nature Reviews Cancer 12, 252-264 (2012).
4. Robert, C. et al. Pembrolizumab versus Ipilimumab in Advanced Melanoma. N. Engl. J. Med. 372, 150419053123009 (2015).
5. Simon, S. & Labarriere, N. PD-1 expression on tumor-specific T cells: Friend or foe for immunotherapy? Oncolmmunology 7, e1364828 (2017).
6. June, C. H., Warshauer, J. T. & Bluestone, J. A. Is autoimmunity the Achilles' heel of cancer immunotherapy? Nature Medicine 23, 540-547 (2017).
7. Nizard, M. et al. Induction of resident memory T cells enhances the efficacy of cancer vaccine. Nat. Commun. 8, 15221 (2017).
8. Malik, B. T. et al. Resident memory T cells in the skin mediate durable immunity to melanoma. Sci. Immunol. 2, (2017).
9. Ganesan, A.-P. et al. Tissue-resident memory features are linked to the magnitude of cytotoxic T cell responses in human lung cancer. Nat. Immunol. 18, 940-950 (2017).
10. Djenidi, F. et al. CD8+CD103+ tumor-infiltrating lymphocytes are tumor-specific tissue-resident memory T cells and a prognostic factor for survival in lung cancer patients. J. Immunol. 194, 3475-86 (2015).
11. Schenkel, J. M. & Masopust, D. Tissue-resident memory T cells. Immunity 41, 886-97 (2014).
12. Mackay, L. K. et al. The developmental pathway for CD103(+)CD8+ tissue-resident memory T cells of skin. Nat. Immunol. 14, 1294-301 (2013).
13. Hombrink, P. et al. Programs for the persistence, vigilance and control of human CD8+lung-resident memory T cells. Nat. Immunol. 17, 1467-1478 (2016).
14. Milner, J. J. et al. Runx3 programs CD8+ T cell residency in non-lymphoid tissues and tumours. Nature 2017 (2017). doi:10.1038/nature24993
15. Cheuk, S. et al. CD49a Expression Defines Tissue-Resident CD8(+) T Cells Poised for Cytotoxic Function in Human Skin. Immunity 46, 287-300 (2017).
16. Shwetank et al. Maintenance of PD-1 on brain-resident memory CD8 T cells is antigen independent. Immunol. Cell Biol. 95, 953-959 (2017).
17. Prasad, S. et al. The PD-1: PD-L1 pathway promotes development of brain-resident memory T cells following acute viral encephalitis. J. Neuroinflammation 14, 82 (2017).
18. Collins, S. et al. Regulation of CD4+ and CD8+ Effector Responses by Sprouty-1. PLoS One 7, e49801 (2012).
19. Chan, C. J. et al. The receptors CD96 and CD226 oppose each other in the regulation of natural killer cell functions. Nat. Immunol. 15, 431-438 (2014).
20. Janakiram, M., Chinai, J. M., Zhao, A., Sparano, J. A. & Zang, X. HHLA2 and TMIGD2: new immunotherapeutic targets of the B7 and CD28 families. doi:10.1080/2162402X.2015.1026534
21. Utting, O. et al. Immune Functions in Mice Lacking Clnk, an SLP-76-Related Adaptor Expressed in a Subset of Immune Cells. Mol. Cell. Biol. 24, 6067-6075 (2004).
22. Rapaport, A. S. et al. The Inhibitory Receptor NKG2A Sustains Virus-Specific CD8+ T Cells in Response to a Lethal Poxvirus Infection. Immunity 43, 1112-1124 (2015).
23. Pallett, L. J. et al. IL-2(high) tissue-resident T cells in the human liver: Sentinels for hepatotropic infection. J. Exp. Med. 214, 1567-1580 (2017).
24. Zheng, C. et al. Landscape of Infiltrating T Cells in Liver Cancer Revealed by Single-Cell Sequencing. Cell 169, 1342-1356.e16 (2017).
25. Bindea, G. et al. Spatiotemporal dynamics of intratumoral immune cells reveal the immune landscape in human cancer. Immunity 39, 782-95 (2013).
26. Honey, K. CCL3 and CCL4 actively recruit CD8+ T cells. Nat. Rev. Immunol. 6, 427 (2006).
27. Croft, M., So, T., Duan, W. & Soroosh, P. The significance of OX40 and OX40L to T-cell biology and immune disease. Immunol. Rev. 229, 173-91 (2009).
28. Kniemeyer, O., Brakhage, A. A., Ferreira, F., Wallner, M. & Sawitzki, B. Regulatory T Cell Specificity Directs Tolerance versus Allergy against Aeroantigens in Humans. Cell 167, 1067-1078.e16 (2016).
29. Macosko, E. Z. et al. Highly Parallel Genome-wide Expression Profiling of Individual Cells Using Nanoliter Droplets. Cell 161, 1202-1214 (2015).
30. Trapnell, C. et al. The dynamics and regulators of cell fate decisions are revealed by pseudotemporal ordering of single cells. Nat. Biotechnol. 32, 381-6 (2014).
31. Patil, V. S. et al. Precursors of human CD4+ cytotoxic T lymphocytes identified by single-cell transcriptome analysis. Sci. Immunol 3, (2018).
32. Emgård, J. et al. Oxysterol Sensing through the Receptor GPR183 Promotes the Lymphoid-Tissue-Inducing Function of Innate Lymphoid Cells and Colonic Inflammation. Immunity 48, 120-132.e8 (2018).
33. Aranda, J. F. et al. MYADM regulates Rac1 targeting to ordered membranes required for cell spreading and migration. Mol. Biol. Cell 22, 1252-1262 (2011).
34. Nieminen, M. et al. Vimentin function in lymphocyte adhesion and transcellular migration. Nat. Cell Biol. 8, 156-162 (2006).
35. Tachibana, M. et al. Ankyrin repeat domain 28 (ANKRD28), a novel binding partner of DOCK180, promotes cell migration by regulating focal adhesion formation. Exp. Cell Res. 315, 863-876 (2009).
36. Stinchcombe, J. C. et al. Rab27a is required for regulated secretion in cytotoxic T lymphocytes. J. Cell Biol. 152, 825-833 (2001).
37. Franciszkiewicz, K. et al. CD103 or LFA-1 engagement at the immune synapse between cytotoxic T cells and tumor cells promotes maturation and regulates T-cell effector functions. Cancer Res. 73, 617-628 (2013).
38. Yang, C. Y. et al. The transcriptional regulators Id2 and Id3 control the formation of distinct memory CD8+ T cell subsets. Nat. Immunol. 12,1221-1229 (2011).
39. Cui, W., Liu, Y., Weinstein, J. S., Craft, J. & Kaech, S. M. An interleukin-21-interleukin-10-STAT3 pathway is critical for functional maturation of memory CD8+ T cells. Immunity 35, 792-805 (2011).
40. Dominguez, C. X. et al. The transcription factors ZEB2 and T-bet cooperate to program cytotoxic T cell terminal differentiation in response to LCMV viral infection. J. Exp. Med. (2015). doi:10.1084/jem.20150186
41. Muthusamy, N., Barton, K. & Leiden, J. M. Defective activation and survival of T cells lacking Ets-1 transcription factor. Nature 377, 639-642 (1995).
42. Mackay, L. K. et al. Hobit and Blimp 1 instruct a universal transcriptional program of tissue residency in lymphocytes. Science (80-.). 352, 459-463 (2016).
43. Xiao, Y. et al. Protein Tyrosine Phosphatase SHP-1 Modulates T Cell Responses by Controlling Cbl-b Degradation. J. Immunol. 195, 4218-27 (2015).
44. Huang, C.-Y. et al. DUSP4 deficiency enhances CD25 expression and CD4+ T-cell proliferation without impeding T-cell development. Eur. J. Immunol. 42, 476-488 (2012).
45. Emadali, A. et al. Haploinsufficiency for NR3C1, the gene encoding the glucocorticoid receptor, in blastic plasmacytoid dendritic cell neoplasms. Blood 127, 3040-3053 (2016).
46. Engler, J. B. et al. Glucocorticoid receptor in T cells mediates protection from autoimmunity in pregnancy. Proc. Natl. Acad. Sci. 114, E181-E190 (2017).
47. Prasad, S. et al. The PD-1: PD-L1 pathway promotes development of brain-resident memory T cells following acute viral encephalitis. J. Neuroinflammation 14, 82 (2017).
48. Kim, S. V. et al. GPR15-Mediated Homing Controls Immune Homeostasis in the Large Intestine Mucosa. Science (80-.). 340, 1456-1459 (2013).
49. Witherden, D. A. et al. The junctional adhesion molecule JAML is a costimulatory receptor for epithelial γδ T cell activation. Science (80-.). 329, 1205-1210 (2010).
50. Bacon, C., Endris, V. & Rappold, G. A. The cellular function of srGAP3 and its role in neuronal morphogenesis. Mechanisms of Development 130, 391-395 (2013).
51. Pardoll D M. The blockade of immune checkpoints in cancer immunotherapy. Nature reviews Cancer 2012; 12: 252-264.
52. Drake C G, Lipson E J, Brahmer J R. Breathing new life into immunotherapy: review of melanoma, lung and kidney cancer. Nature reviews Clinical oncology 2014; 11: 24-37.
53. Sharma P, Allison J P. Immune checkpoint targeting in cancer therapy: toward combination strategies with curative potential. Cell 2015; 161: 205-214.
54. Anusha-Preethi Ganesan J C, Oliver Wood, Eva M Garrido-Martin, Serena Chee, Toby Mellows, Daniela Samaniego-Castruita, Divya Singh, Gregory Seumois, Aiman Alzetani, Edwin Woo, Peter S. Friedmann, G J Thomas, Emma V King, Tilman Sanchez-Elsner, Pandurangan Vijayanand*#, Christian H Ottensmeier*, *joint senior authors #corresponding author. Tissue-resident memory features are linked to the magnitude of cytotoxic T cell responses in human lung cancer. Nature Immunology—in press 2017;
55. Engel I, Seumois G, Chavez L, Samaniego-Castruita D, White B, Chawla A, Mock D, Vijayanand P, Kronenberg M. Innate-like functions of natural killer T cell subsets result from highly divergent gene programs. Nat Immunol 2016; 17: 728-+.
56. Schmiedel B J, Seumois G, Samaniego-Castruita D, Cayford J, Schulten V, Chavez L, Ay F, Sette A, Peters B, Vijayanand P. 17q21 asthma-risk variants switch CTCF binding and regulate IL-2 production by T cells. Nature communications 2016; 7:
57. Seumois G, Chavez L, Gerasimova A, Lienhard M, Omran N, Kalinke L, Vedanayagam M, Ganesan A P V, Chawla A, Djukanovic R, Ansel K M, Peters B, et al. Epigenomic analysis of primary human T cells reveals enhancers associated with T(H)2 memory cell differentiation and asthma susceptibility. Nat Immunol 2014; 15: 777-+.
58. LaFlam T N, Seumois G, Miller C N, Lwin W, Fasano K J, Waterfield M, Proekt I, Vijayanand P, Anderson M S. Identification of a novel cis-regulatory element essential for immune tolerance. J Exp Med 2015; 212: 1993-2002.
59. Yue X, Trifari S, Aijo T, Tsagaratou A, Pastor W A, Zepeda-Martinez J A, Lio C J, Li X, Huang Y, Vijayanand P, Landesmaki H, Rao A. Control of Foxp3 stability through modulation of TET activity. J Exp Med 2016;
60. Vijayanand P, Seumois G, Simpson L J, Abdul-Wajid S, Baumjohann D, Panduro M, Huang X, Interlandi J, Djuretic I M, Brown D R, Sharpe A H, Rao A, et al. Interleukin-4 Production by Follicular Helper T Cells Requires the Conserved 114 Enhancer Hypersensitivity Site V. Immunity 2012;
61. Seumois G, Zapardiel-Gonzalo J, White B, Singh D, Schulten V, Dillon M, Hinz D, Broide D H, Sette A, Peters B, Vijayanand P. Transcriptional Profiling of Th2 Cells Identifies Pathogenic Features Associated with Asthma. Journal of Immunology 2016; 197: 655-664.
62. Arlehamn C L, Seumois G, Gerasimova A, Huang C, Fu Z, Yue X, Sette A, Vijayanand P, Peters B. Transcriptional profile of tuberculosis antigen-specific T cells reveals novel multifunctional features. J Immunol 2014; 193: 2931-2940.
63. Gerasimova A, Chavez L, Li B, Seumois G, Greenbaum J, Rao A, Vijayanand P, Peters B. Predicting cell types and genetic variations contributing to disease by combining GWAS and epigenetic data. PLoS One 2013; 8: e54359.
64. Hinz D, Seumois G, Gholami A M, Greenbaum J A, Lane J, White B, Broide D H, Schulten V, Sidney J, Bakhru P, Oseroff C, Wambre E, et al. Lack of allergy to timothy grass pollen is not a passive phenomenon but associated with the allergen-specific modulation of immune reactivity. Clinical and Experimental Allergy 2016; 46: 705-719.
65. Seumois G, Vijayanand P, Eisley C J, Omran N, Kalinke L, North M, Ganesan A P, Simpson L J, Hunkapiller N, Moltzahn F, Woodruff P G, Fahy J V, et al. An integrated nano-scale approach to profile miRNAs in limited clinical samples. Am J Clin Exp Immunol 2012; 1: 70-89.
66. Vijayanand P, Durkin K, Hartmann G, Morjaria J, Seumois G, Staples K J, Hall D, Bessant C, Bartholomew M, Howarth P H, Friedmann P S, Djukanovic R. Chemokine receptor 4 plays a key role in T cell recruitment into the airways of asthmatic patients. Journal of immunology 2010; 184: 4568-4574.
67. Vijayanand P, Seumois G, Pickard C, Powell R M, Angco G, Sammut D, Gadola S D, Friedmann P S, Djukanovic R. Invariant natural killer T cells in asthma and chronic obstructive pulmonary disease. The New England journal of medicine 2007; 356: 1410-1422.
68. Patil V, Madrigal A, Schmiedel B, Clarke J, de Silva A, Harris E, Peters B, Seumois G, Weiskopf D, Sette A, Vijayanand P. Precursors of human CD4+ cytotoxic T lymphocytes identified by single-cell transcriptome analysis. Science Immunology—under review 2017;
69. Mellone M, Hanley C J, Thirdborough S, Mellows T, Garcia E, Woo J, Tod J, Frampton S, Jenei V, Moutasim K A, Kabir T D, Brennan P A, et al. Induction of fibroblast senescence generates a non-fibrogenic myofibroblast phenotype that differentially impacts on cancer prognosis. Aging 2016; 9: 114-132.
70. Wood O, Woo J, Seumois G, Savelyeva N, McCann K J, Singh D, Jones T, Peel L, Breen M S, Ward M, Garrido Martin E, Sanchez-Elsner T, et al. Gene expression analysis of TIL rich HPV-driven head and neck tumors reveals a distinct B-cell signature when compared to HPV independent tumors. Oncotarget 2016; 7: 56781-56797.
71. Seckl M J, Ottensmeier C H, Cullen M, Schmid P, Ngai Y, Muthukumar D, Thompson J, Harden S, Middleton G, Fife K M, Crosse B, Taylor P, et al. Multicenter, Phase III, Randomized, Double-Blind, Placebo-Controlled Trial of Pravastatin Added to First-Line Standard Chemotherapy in Small-Cell Lung Cancer (LUNGSTAR). J Clin Oncol 2017; JCO2016697391.
72. Ganesan A P, Wood O, Garrido-Martin E M, Chee S, Mellows T, Clarke J, Samaniego-Castruita D, Singh D, Seumois G, Altezani A, Woo E, Friedmann P S, et al. Tissue-resident memory features are linked to the magnitude of cytotoxic T cell responses in human lung cancer. Nature immunology 2017; in press:
73. Ottensmeier C H, Perry K L, Harden E L, Stasakova J, Jenei V, Fleming J, Wood O, Woo J, Woelk C H, Thomas G J, Thirdborough S M. Upregulated Glucose Metabolism Correlates Inversely with CD8+ T-cell Infiltration and Survival in Squamous Cell Carcinoma. Cancer Res 2016; 76: 4136-4148.
74. Noble F, Mellows T, McCormick Matthews L H, Bateman A C, Harris S, Underwood T J, Byrne J P, Bailey I S, Sharland D M, Kelly J J, Primrose J N, Sahota S S, et al. TumApplicants' infiltrating lymphocytes correlate with improved survival in patients with oesophageal adenocarcinoma. Cancer Immunol Immunother 2016;
75. McCann K J, Mander A, Cazaly A, Chudley L, Stasakova J, Thirdborough S M, King A, Lloyd-Evans P, Buxton E, Edwards C, Halford S, Bateman A, et al. Targeting Carcinoembryonic Antigen with DNA Vaccination: On-Target Adverse Events Link with Immunologic and Clinical Outcomes. Clin Cancer Res 2016;
76. Karydis I, Chan P Y, Wheater M, Arriola E, Szlosarek P W, Ottensmeier C H. Clinical activity and safety of Pembrolizumab in Ipilimumab pre-treated patients with uveal melanoma. Oncoimmunology 2016; 5: e1143997.
77. Chandran P A, Laske K, Cazaly A, Rusch E, Schmid-Horch B, Rammensee H G, Ottensmeier C H, Gouttefangeas C. Validation of immunomonitoring methods for application in clinical studies: The HLA-peptide multimer staining assay. Cytometry B Clin Cytom 2016;
78. Arriola E, Wheater M, Galea I, Cross N, Maishman T, Hamid D, Stanton L, Cave J, Geldart T, Mulatero C, Potter V, Danson S, et al. Outcome and Biomarker Analysis from a Multicenter Phase 2 Study of Ipilimumab in Combination with Carboplatin and Etoposide as First-Line Therapy for Extensive-Stage SCLC. J Thorac Oncol 2016;
79. McCann K J, Godeseth R, Chudley L, Mander A, Di Genova G, Lloyd-Evans P, Kerr J P, Malykh V B, Jenner M W, Orchard K H, Stevenson F K, Ottensmeier C H. Idiotypic DNA vaccination for the treatment of multiple myeloma: safety and immunogenicity in a phase I clinical study. Cancer Immunol Immunother 2015; 64: 1021-1032.
80. Johnson P, Challis R, Chowdhury F, Gao Y, Harvey M, Geldart T, Kerr P, Chan C, Smith A, Steven N, Edwards C, Ashton-Key M, et al. Clinical and biological effects of an agonist anti-CD40 antibody: a Cancer Research U K phase I study. Clin Cancer Res 2015; 21: 1321-1328.
81. Ward M J, Thirdborough S M, Mellows T, Riley C, Harris S, Suchak K, Webb A, Hampton C, Patel N N, Randall C J, Cox H J, Jogai S, et al. Tumour-infiltrating lymphocytes predict for outcome in HPV-positive oropharyngeal cancer. British journal of cancer 2014; 110: 489-500.
82. Kvistborg P, Philips D, Kelderman S, Hageman L, Ottensmeier C, Joseph-Pietras D, Welters M J, van der Burg S, Kapiteijn E, Michielin O, Romano E, Linnemann C, et al. Anti-CTLA-4 therapy broadens the melanoma-reactive CD8+ T cell response. Science translational medicine 2014; 6: 254ra128.
83. Chudley L, McCann K J, Coleman A, Cazaly A M, Bidmon N, Britten C M, van der Burg S H, Gouttefangeas C, Jandus C, Laske K, Maurer D, Romero P, et al. Harmonisation of short-term in vitro culture for the expansion of antigen-specific CD8(+) T cells with detection by ELISPOT and HLA-multimer staining. Cancer Immunol Immunother 2014; 63: 1199-1211.
84. Chudley L, McCann K, Mander A, Tjelle T, Campos-Perez J, Godeseth R, Creak A, Dobbyn J, Johnson B, Bass P, Heath C, Kerr P, et al. DNA fusion-gene vaccination in patients with prostate cancer induces high-frequency CD8(+) T-cell responses and increases PSA doubling time. Cancer Immunol Immunother 2012;
85. Britten C M, Janetzki S, Butterfield L H, Ferrari G, Gouttefangeas C, Huber C, Kalos M, Levitsky H I, Maecker H T, Melief C J, O'Donnell-Tormey J, Odunsi K, et al. T cell assays and MIATA: the essential minimum for maximum impact. Immunity 2012; 37: 1-2.
86. Hodi F S, O'Day S J, McDermott D F, Weber R W, Sosman J A, Haanen J B, Gonzalez R, Robert C, Schadendorf D, Hassel J C, Akerley W, van den Eertwegh A J, et al. Improved survival with ipilimumab in patients with metastatic melanoma. N Engl J Med 2010; 363: 711-723.
87. McCann K J, Ashton-Key M, Smith K, Stevenson F K, Ottensmeier C H. Primary central nervous system lymphoma: tumor-related clones exist in the blood and bone marrow with evidence for separate development. Blood 2009; 113: 4677-4680.
88. Mander A, Chowdhury F, Low L, Ottensmeier C H. Fit for purpose? A case study: validation of immunological endpoint assays for the detection of cellular and humoral responses to anti-tumApplicants' DNA fusion vaccines. Cancer Immunol Immunother 2009; 58: 789-800.
89. Low L, Mander A, McCann K, Dearnaley D, Tjelle T, Mathiesen I, Stevenson F, Ottensmeier C H. DNA vaccination with electroporation induces increased antibody responses in patients with prostate cancer. Human gene therapy 2009; 20: 1269-1278.
90. Lee S M, Rudd R, Woll P J, Ottensmeier C, Gilligan D, Price A, Spiro S, Gower N, Jitlal M, Hackshaw A. Randomized double-blind placebo-controlled trial of thalidomide in combination with gemcitabine and Carboplatin in advanced non-small-cell lung cancer. J Clin Oncol 2009; 27: 5248-5254.
91. Rice J, Ottensmeier C H, Stevenson F K. DNA vaccines: precision tools for activating effective immunity against cancer. Nature reviews 2008; 8: 108-120.
92. McCann K J, Johnson P W, Stevenson F K, Ottensmeier C H. Universal N-glycosylation sites introduced into the B-cell receptor of follicular lymphoma by somatic mutation: a second tumorigenic event? Leukemia: official journal of the Leukemia Society of America, Leukemia Research Fund, UK 2006; 20: 530-534.
93. McCann K, Sahota S S, Stevenson F K, Ottensmeier C H. Idiotype gene rescue in follicular lymphoma. Methods Mol Med 2005; 115: 145-171.
94. Ottensmeier C H, Stevenson F K. Isotype switch variants reveal clonally related subpopulations in diffuse large B-cell lymphoma. Blood 2000; 96: 2550-2556.
95. Ottensmeier C H, Wilkins B S, Stevenson F K. Immunogenetic features of diffuse and follicular lymphoma vary with disease status and reveal multiple isotype expression. Blood 1997; 90: 3932-3932.
96. Ottensmeier C, Swanson L, Strobel T, Druker B, NiloffJ, Cannistra S A. Absence of constitutive EGF receptor activation in ovarian cancer cell lines. British journal of cancer 1996; 74: 446-452.
97. Ottensmeier C, Mead G. Histological transformation of indolent (follicular) lymphoma. Ann Oncol 1996; 7: 849-853.
98. Cannistra S A, DeFranzo B, Niloff J, Ottensmeier C. Functional heterogeneity of CD44 molecules in ovarian cancer cell lines. Clinical Cancer Res 1995; 1: 333-342.
99. Cannistra S A, Abu-Jawdeh G, Niloff J, Strobel T, Swanson L, Andersen J, Ottensmeier C. CD44 variant expression is a common feature of epithelial ovarian cancer: lack of association with standard prognostic factors. J Clin Oncol 1995; 13: 1912-1921.
100. Cannistra S A, Kansas G S, NiloffJ, DeFranzo B, Kim Y, Ottensmeier C. Binding of ovarian cancer cells to peritoneal mesothelium in vitro is partly mediated by CD44H. Cancer Res 1993; 53: 3830-3838.
101. Ganesan A P, Clarke J, Wood O, Garrido-Martin E M, Chee S J, Mellows T, Samaniego-Castruita D, Singh D, Seumois G, Alzetani A, Woo E, Friedmann P S, et al. Tissue-resident memory features are linked to the magnitude of cytotoxic T cell responses in human lung cancer. Nat Immunol 2017; 18: 940-950.
102. Yu B, Zhang K, Milner J J, Toma C, Chen R, Scott-Browne J P, Pereira R M, Crotty S, Chang J T, Pipkin M E, Wang W, Goldrath A W. Epigenetic landscapes reveal transcription factors that regulate CD8(+) T cell differentiation. Nature immunology 2017; 18: 573-582.
103. Overwijk W W, Theoret M R, Finkelstein S E, Surman D R, de Jong L A, Vyth-Dreese F A, Dellemijn T A, Antony P A, Spiess P J, Palmer D C, Heimann D M, Klebanoff C A, et al. Tumor regression and autoimmunity after reversal of a functionally tolerant state of self-reactive CD8+ T cells. J Exp Med 2003; 198: 569-580.
104. Helmich B K, Dutton R W. The role of adoptively transferred CD8 T cells and host cells in the control of the growth of the EG7 thymoma: factors that determine the relative effectiveness and homing properties of Tcl and Tc2 effectors. J Immunol 2001; 166: 6500-6508.
105. Thompson E D, Enriquez H L, Fu Y X, Engelhard V H. Tumor masses support naïve T cell infiltration, activation, and differentiation into effectors. Journal of Experimental Medicine 2010; 207: 1791-1804.
106. Lu T, Ramakrishnan R, Altiok S, Youn J I, Cheng P, Celis E, Pisarev V, Sherman S, Sporn M B, Gabrilovich D. Tumor-infiltrating myeloid cells induce tumor cell resistance to cytotoxic T cells in mice. J Clin Invest 2011; 121: 4015-4029.
107. Chen R, Belanger S, Frederick M A, Li B, Johnston R I, Xiao N, Liu Y C, Sharma S, Peters B, Rao A, Crotty S, Pipkin M E. In vivo RNA interference screens identify regulators of antiviral CD4(+) and CD8(+) T cell differentiation. Immunity 2014; 41: 325-338.
108. Trifari S, Pipkin M E, Bandukwala H S, Aijo T, Bassein J, Chen R, Martinez G J, Rao A. MicroRNA-directed program of cytotoxic CD8+ T-cell differentiation. Proc Natl Acad Sci USA 2013; 110: 18608-18613.
109. Galon J, Costes A, Sanchez-Cabo F, Kirilovsky A, Mlecnik B, Lagorce-Pages C, Tosolini M, Camus M, Berger A, Wind P, Zinzindohoue F, Bruneval P, et al. Type, density, and location of immune cells within human colorectal tumors predict clinical outcome. Science 2006; 313: 1960-1964.
110. Yang Y. Cancer immunotherapy: harnessing the immune system to battle cancer. J Clin Invest 2015; 125: 3335-3337.
111. Mackay L K, Minnich M, Kragten N A, Liao Y, Nota B, Seillet C, Zaid A, Man K, Preston S, Freestone D, Braun A, Wynne-Jones E, et al. Hobit and Blimp 1 instruct a universal transcriptional program of tissue residency in lymphocytes. Science 2016; 352: 459-463.
112. Sathaliyawala T, Kubota M, Yudanin N, Turner D, Camp P, Thome J J, Bickham K L, Lerner H, Goldstein M, Sykes M, Kato T, Farber D L. Distribution and compartmentalization of human circulating and tissue-resident memory T cell subsets. Immunity 2013; 38: 187-197.
113. Garon E B, Rizvi N A, Hui R, Leighl N, Balmanoukian A S, Eder J P, Patnaik A, Aggarwal C, Gubens M, Horn L, Carcereny E, Ahn M J, et al. Pembrolizumab for the treatment of non-small-cell lung cancer. The New England journal of medicine 2015; 372: 2018-2028.
114. Miller J F, Sadelain M. The journey from discoveries in fundamental immunology to cancer immunotherapy. Cancer Cell 2015; 27: 439-449.
115. Tumeh P C, Harview C L, Yearley J H, Shintaku I P, Taylor E J, Robert L, Chmielowski B, Spasic M, Henry G, Ciobanu V, West A N, Carmona M, et al. PD-1 blockade induces responses by inhibiting adaptive immune resistance. Nature 2014; 515: 568-571.
116. Kim P S, Ahmed R. Features of responding T cells in cancer and chronic infection. Curr Opin Immunol 2010; 22: 223-230.
117. Philip M, Fairchild L, Sun L, Horste E L, Camara S, Shakiba M, Scott A C, Viale A, Lauer P, Merghoub T, Hellmann M D, Wolchok J D, et al. Chromatin states define tumour-specific T cell dysfunction and reprogramming. Nature 2017; 545: 452-456.
118. Huang A C, Postow M A, Orlowski R I, Mick R, Bengsch B, Manne S, Xu W, Harmon S, Giles J R, Wenz B, Adamow M, Kuk D, et al. T-cell invigoration to tumApplicants' burden ratio associated with anti-PD-1 response. Nature 2017; 545: 60-65.
119. Schietinger A, Philip M, Krisnawan V E, Chiu E Y, Delrow J J, Basom R S, Lauer P, Brockstedt D G, Knoblaugh S E, Hammerling G J, Schell T D, Garbi N, et al. Tumor-Specific T Cell Dysfunction Is a Dynamic Antigen-Driven Differentiation Program Initiated Early during Tumorigenesis. Immunity 2016; 45: 389-401.
120. Pauken K E, Sammons M A, Odorizzi P M, Manne S, Godec J, Khan O, Drake A M, Chen Z, Sen D R, Kurachi M, Barnitz R A, Bartman C, et al. Epigenetic stability of exhausted T cells limits durability of reinvigoration by PD-1 blockade. Science 2016; 354: 1160-1165.
121. Akhtar-Zaidi B, Cowper-Sal-lari R, Corradin O, Saiakhova A, Bartels C F, Balasubramanian D, Myeroff L, Lutterbaugh J, Jarrar A, Kalady M F, Willis J, Moore J H, et al. Epigenomic enhancer profiling defines a signature of colon cancer. Science 2012; 336: 736-739.
122. Zhang J A, Mortazavi A, Williams B A, Wold B J, Rothenberg E V. Dynamic transformations of genome-wide epigenetic marking and transcriptional control establish T cell identity. Cell 2012; 149: 467-482.
123. Bindea G, Mlecnik B, Tosolini M, Kirilovsky A, Waldner M, Obenauf A C, Angell H, Fredriksen T, Lafontaine L, Berger A, Bruneval P, Fridman W H, et al. Spatiotemporal dynamics of intratumoral immune cells reveal the immune landscape in human cancer. Immunity 2013; 39: 782-795.
124. Baitsch L, Baumgaertner P, Devevre E, Raghav S K, Legat A, Barba L, Wieckowski S, Bouzourene H, Deplancke B, Romero P, Rufer N, Speiser D E. Exhaustion of tumor-specific CD8(+) T cells in metastases from melanoma patients. The Journal of clinical investigation 2011; 121: 2350-2360.
125. Collins N, Godec J, Zou L H, Mihm M C, Getz G, Haining W N. Transcriptional Hallmarks Of Tumor Infiltrating Lymphocyte Responses To Melanoma. Blood 2013; 122:
126. Tirosh I, Izar B, Prakadan S M, Wadsworth M H, 2nd, Treacy D, Trombetta J J, Rotem A, Rodman C, Lian C, Murphy G, Fallahi-Sichani M, Dutton-Regester K, et al. Dissecting the multicellular ecosystem of metastatic melanoma by single-cell RNA-seq. Science 2016; 352: 189-196.
127. Curran M A, Geiger T L, Montalvo W, Kim M, Reiner S L, Al-Shamkhani A, Sun J C, Allison J P. Systemic 4-1B B activation induces a novel T cell phenotype driven by high expression of Eomesodermin. J Exp Med 2013; 210: 743-755.
128. Willoughby J E, Kerr J P, Rogel A, Taraban V Y, Buchan S L, Johnson P W, Al-Shamkhani A. Differential impact of CD27 and 4-1B B costimulation on effector and memory CD8 T cell generation following peptide immunization. J Immunol 2014; 193: 244-251.
129. Topalian S L, Drake C G, Pardoll D M. Immune checkpoint blockade: a common denominator approach to cancer therapy. Cancer Cell 2015; 27: 450-461.
130. Wolff M, Kuball J, Ho W Y, Nguyen H, Manley T J, Bleakley M, Greenberg P D. Activation-induced expression of CD137 permits detection, isolation, and expansion of the full repertoire of CD8+ T cells responding to antigen without requiring knowledge of epitope specificities. Blood 2007; 110: 201-210.
131. Gros A, Robbins P F, Yao X, Li Y F, Turcotte S, Tran E, Wunderlich J R, Mixon A, Farid S, Dudley M E, Hanada K, Almeida J R, et al. PD-1 identifies the patient-specific CD8(+) tumor-reactive repertoire infiltrating human tumors. The Journal of clinical investigation 2014; 124: 2246-2259.
132. Fourcade J, Sun Z, Benallaoua M, Guillaume P, Luescher I F, Sander C, Kirkwood J M, Kuchroo V, ZarApplicants' H M. Upregulation of Tim-3 and PD-1 expression is associated with tumor antigen-specific CD8+ T cell dysfunction in melanoma patients. J Exp Med 2010; 207: 2175-2186.
133. Wherry E J, Ha S J, Kaech S M, Haining W N, Sarkar S, Kalia V, Subramaniam S, Blattman J N, Barber D L, Ahmed R. Molecular signature of CD8+ T cell exhaustion during chronic viral infection. Immunity 2007; 27: 670-684.
134. Mackay L K, RahimpApplicants' A, Ma J Z, Collins N, Stock A T, Hafon M L, Vega-Ramos J, Lauzurica P, Mueller S N, Stefanovic T, Tscharke D C, Heath W R, et al. The developmental pathway for CD103(+)CD8+ tissue-resident memory T cells of skin. Nature immunology 2013; 14: 1294-1301.
135. Mueller S N, Gebhardt T, Carbone F R, Heath W R. Memory T cell subsets, migration patterns, and tissue residence. Annual review of immunology 2013; 31: 137-161.
136. Skon C N, Lee J Y, Anderson K G, Masopust D, Hogquist K A, Jameson S C. Transcriptional downregulation of S1prl is required for the establishment of resident memory CD8+ T cells. Nature immunology 2013; 14: 1285-1293.
137. Best J A, Blair D A, Knell J, Yang E, Mayya V, Doedens A, Dustin M L, Goldrath A W, Immunological Genome Project C. Transcriptional insights into the CD8(+) T cell response to infection and memory T cell formation. Nat Immunol 2013; 14: 404-412.
138. Creyghton M P, Cheng A W, Welstead G G, Kooistra T, Carey B W, Steine E J, Hanna J, Lodato M A, Frampton G M, Sharp P A, Boyer L A, Young R A, et al. Histone H3K27ac separates active from poised enhancers and predicts developmental state. P Natl Acad Sci USA 2010; 107: 21931-21936.
139. Wei G, Wei L, Zhu J, Zang C, Hu-Li J, Yao Z, Cui K, Kanno Y, Roh T Y, Watford W T, Schones D E, Peng W, et al. Global mapping of H3K4me3 and H3K27me3 reveals specificity and plasticity in lineage fate determination of differentiating CD4+ T cells. Immunity 2009; 30: 155-167.
140. Buenrostro J D, Wu B, Chang H Y, Greenleaf W J. ATAC-seq: A Method for Assaying Chromatin Accessibility Genome-Wide. Current protocols in molecular biology/edited by Frederick M Ausubel [et al] 2015; 109: 21 29 21-29.
141. Debey S, Schoenbeck U, Hellmich M, Gathof B S, Pillai R, Zander T, Schultze J L. Comparison of different isolation techniques prior gene expression profiling of blood derived cells: impact on physiological responses, on overall expression and the role of different cell types. The pharmacogenomics journal 2004; 4: 193-207.
142. Seumois G, Chavez L, Gerasimova A, Lienhard M, Omran N, Kalinke L, Vedanayagam M, Ganesan A P, Chawla A, Djukanovic R, Ansel K M, Peters B, et al. Epigenomic analysis of primary human T cells reveals enhancers associated with TH2 memory cell differentiation and asthma susceptibility. Nat Immunol 2014; 15: 777-788.
143. Hanna R N, Cekic C, Sag D, Tacke R, Thomas G D, Nowyhed H, Henley E, Rasquinha N, McArdle S, Wu R, Peluso E, Metzger D, et al. Patrolling monocytes control tumor metastasis to the lung. Science 2015; 350: 985-990.
144. Ma W, Ay F, Lee C, Gulsoy G, Deng X, Cook S, Hesson J, Cavanaugh C, Ware C B, Krumm A, Shendure J, Blau C A, et al. Fine-scale chromatin interaction maps reveal the cis-regulatory landscape of human lincRNA genes. Nat Methods 2015; 12: 71-78.
145. Libbrecht M W, Ay F, Hoffman M M, Gilbert D M, Bilmes J A, Noble W S. Joint annotation of chromatin state and chromatin conformation reveals relationships among domain types and identifies domains of cell-type-specific expression. Genome Res 2015; 25: 544-557.
146. Gittelman R M, Hun E, Ay F, Madeoy J, Pennacchio L, Noble W S, Hawkins R D, Akey J M. Comprehensive identification and analysis of human accelerated regulatory DNA. Genome Res 2015; 25: 1245-1255.
147. Dileep V, Ay F, Sima J, Vera D L, Noble W S, Gilbert D M. Topologically associating domains and their long-range contacts are established during early G1 coincident with the establishment of the replication-timing program. Genome Res 2015; 25: 1104-1113.
148. Ay F, Vu T H, Zeitz M J, Varoquaux N, Carette J E, Vert J P, Hoffivan A R, Noble W S. Identifying multi-locus chromatin contacts in human cells using tethered multiple 3C. BMC genomics 2015; 16: 121.
149. Ay F, Noble W S. Analysis methods for studying the 3D architecture of the genome. Genome Biol 2015; 16: 183.
150. Ay F, Bunnik E M, Varoquaux N, Bol S M, Prudhomme J, Vert J P, Noble W S, Le Roch K G. Three-dimensional modeling of the P. falciparum genome during the erythrocytic cycle reveals a strong connection between genome architecture and gene expression. Genome Res 2014; 24: 974-988.
151. Ay F, Bailey T L, Noble W S. Statistical confidence estimation for Hi-C data reveals regulatory chromatin contacts. Genome Res 2014; 24: 999-1011.
152. Zeitz M J, Ay F, Heidmann J D, Lerner P L, Noble W S, Steelman B N, Hoffman A R. Genomic interaction profiles in breast cancer reveal altered chromatin architecture. PLoS One 2013; 8: e73974.
153. Vita R, Peters B, Josephs Z, de Matos P, Ennis M, Turner S, Steinbeck C, SeymApplicants' E, Zarebski L, Sette A. A Model for Collaborative Curation, The IEDB and ChEBI Curation of Non-peptidic Epitopes. Immunome Res 2011; 7: 1-8.
154. Peters B, Sette A. Integrating epitope data into the emerging web of biomedical knowledge resources. Nat Rev Immunol 2007; 7: 485-490.
155. Sette A, Fleri W, Peters B, Sathiamurthy M, Bui R H, Wilson S. A roadmap for the immunomics of category A-C pathogens. Immunity 2005; 22: 155-161.
156. Schulten V, Tripple V, Seumois G, Qian Y, Scheuermann R H, Fu Z, Locci M, Rosales S, Vijayanand P, Sette A, Alam R, Crotty S, et al. Allergen-specific immunotherapy modulates the balance of circulating Tfh and Tfr cells. J Allergy Clin Immunol 2017;
157. Grifoni A, Angelo M, Sidney J, Paul S, Peters B, de Silva A D, Phillips E, Mallal S, Diehl S A, Botten J, Boyson J, Kirkpatrick B D, et al. Patterns of Cellular Immunity Associated with Experimental Infection with rDEN2Delta30 (Tonga/74) Support Its Suitability as a Human Dengue Virus Challenge Strain. J Virol 2017; 91:
158. Angelo M A, Grifoni A, O'Rourke P H, Sidney J, Paul S, Peters B, de Silva A D, Phillips E, Mallal S, Diehl S A, Kirkpatrick B D, Whitehead S S, et al. Human CD4+ T Cell Responses to an Attenuated Tetravalent Dengue Vaccine Parallel Those Induced by Natural Infection in Magnitude, HLA Restriction, and Antigen Specificity. J Virol 2017; 91:
159. Romero P, Banchereau J, Bhardwaj N, Cockett M, Disis M L, Dranoff G, Gilboa E, Hammond S A, Hershberg R, Korman A J, Kvistborg P, Melief C, et al. The Human Vaccines Project: A roadmap for cancer vaccine development. Science translational medicine 2016; 8: 334 ps339.
160. Bono M R, Fernandez D, Flores-Santibanez F, Rosemblatt M, Sauma D. CD73 and CD39 ectonucleotidases in T cell differentiation: Beyond immunosuppression. FEBS letters 2015; 589: 3454-3460.
161. Kuhny M, Hochdorfer T, Ayata C K, Idzko M, Huber M. CD39 is a negative regulator of P2X7-mediated inflammatory cell death in mast cells. Cell communication and signaling: CCS 2014; 12: 40.
162. Sandoval-Montes C, Santos-Argumedo L. CD38 is expressed selectively during the activation of a subset of mature T cells with reduced proliferation but improved potential to produce cytokines. Journal of leukocyte biology 2005; 77: 513-521.
163. Munoz P, Mittelbrunn M, de la Fuente H, Perez-Martinez M, Garcia-Perez A, Ariza-Veguillas A, Malavasi F, Zubiaur M, Sanchez-Madrid F, Sancho J. Antigen-induced clustering of surface CD38 and recruitment of intracellular CD38 to the immunologic synapse. Blood 2008; 111: 3653-3664.
164. Faure M, Long E O. KIR2DL4 (CD158d), an N K cell-activating receptor with inhibitory potential. Journal of immunology 2002; 168: 6208-6214.
165. Rajagopalan S, Long E O. KIR2DL4 (CD158d): An activation receptor for HLA-G. Frontiers in immunology 2012; 3: 258.
166. Wisniewski A, Kowal A, Wyrodek E, Nowak I, Majorczyk E, Wagner M, Pawlak-Adamska E, Jankowska R, Slesak B, Frydecka I, Kusnierczyk P. Genetic polymorphisms and expression of HLA-G and its receptors, KIR2DL4 and LILRB1, in non-small cell lung cancer. Tissue antigens 2015; 85: 466-475.
167. Brooke G, Holbrook J D, Brown M H, Barclay A N. Human lymphocytes interact directly with CD47 through a novel member of the signal regulatory protein (SIRP) family. Journal of immunology 2004; 173: 2562-2570.
168. Piccio L, Vermi W, Boles K S, Fuchs A, Strader C A, Facchetti F, Cella M, Colonna M. Adhesion of human T cells to antigen-presenting cells through SIRPbeta2-CD47 interaction costimulates T-cell proliferation. Blood 2005; 105: 2421-2427.
169. Sharma S, Quintana A, Findlay G M, Mettlen M, Baust B, Jain M, Nilsson R, Rao A, Hogan P G. An siRNA screen for NFAT activation identifies septins as coordinators of store-operated Ca2+ entry. Nature 2013; 499: 238-242.
170. Sharma S, Rao A. RNAi screening: tips and techniques. Nat Immunol 2009; 10: 799-804.
171. Gwack Y, Sharma S, Nardone J, Tanasa B, Iuga A, Srikanth S, Okamura H, Bolton D, Feske S, Hogan P G, Rao A. A genome-wide Drosophila RNAi screen identifies DYRK-family kinases as regulators of NFAT. Nature 2006; 441: 646-650.
172. Hombrink P, Helbig C, Backer R A, Piet B, Oja A E, Stark R, Brasser G, Jongejan A, Jonkers R E, Nota B, Basak O, Clevers H C, et al. Programs for the persistence, vigilance and control of human CD8+ lung-resident memory T cells. Nat Immunol 2016; 17: 1467-1478.
173. Gould S E, Junttila M R, de Sauvage F J. Translational value of mouse models in oncology drug development. Nat Med 2015; 21: 431-439.
174. Overwijk W W, Restifo N P. B16 as a mouse model for human melanoma. Current protocols in immunology/edited by John E Coligan [et al] 2001; Chapter 20: Unit 20 21.
175. Abad J D, Wrzensinski C, Overwijk W, De Witte M A, Jorritsma A, Hsu C, Gattinoni L, Cohen C J, Paulos C M, Palmer D C, Haanen J B, Schumacher T N, et al. T-cell receptor gene therapy of established tumors in a murine melanoma model. Journal of immunotherapy 2008; 31: 1-6.

What is claimed is:

1. A method of treating cancer and/or eliciting an anti-tumor response in a subject comprising administering to the subject an effective amount of a population of T-cells that exhibit higher than or lower than baseline expression of one or more genes set forth in Table 1, Table 2, Table 3, Table 4, Table 5 and/or Table 7, or that express a T-cell receptor comprising at least one of the amino acid sequences set forth in Table 6, thereby treating cancer and/or eliciting an anti-tumor response in the subject.

2. A method of treating cancer and/or eliciting an anti-tumor response in a subject comprising administering to the subject an effective amount of an agent that induces higher than or lower than baseline expression of one or more genes set forth in Table 1, Table 2, Table 3, Table 4, Table 5 and/or Table 7 in T-cells, or a T-cell receptor comprising at least one of the amino acid sequences set forth in Table 6, thereby treating cancer and/or eliciting an anti-tumor response in the subject.

3. A method of treating cancer and/or eliciting an anti-tumor response in a subject or sample comprising administering an effective amount of one or more an agent that induces or inhibits in T-cells activity of one or more proteins encoded by genes set forth in Table 1, Table 2, Table 3, Table 4, Table 5 and/or Table 7 to the subject, thereby treating cancer and/or eliciting an anti-tumor response in the subject.

4. The method of any one of claims 1 to 2, wherein the T-cells are tissue-resident memory cells (TRM) or CD8+ T-cells.

5. The method of claim 4, wherein the T-cells are autologous to the subject being treated.

6. The method of any one of claims 1 to 5, wherein the one or more gene or all of the genes from the group of 4-1BB, PD-1, CD103 or TIM3.

7. The method of any one of claims 1 to 6, wherein baseline expression is normalized mean gene expression.

8. The method of claim 7, wherein higher than baseline expression is at least about a 2-fold increase in expression relative to baseline expression and/or lower than baseline expression is at least about a 2-fold decrease in expression relative to baseline expression.

9. The method of any one of claims 2 to 8, wherein the active agent is an antibody, a small molecule, a protein, a peptide, a ligand mimetic or a nucleic acid.

10. The method of any one of claims 1 to 9, further comprising administering to the subject an effective amount of a cytoreductive therapy.

11. The method of claim 10, wherein the cytoreductive therapy is one or more of chemotherapy, immunotherapy, or radiation therapy.

12. A modified T-cell modified to exhibit higher than or lower than baseline expression of one or more genes set forth in Table 1, Table 2, Table 3, Table 4, Table 5 and/or Table 7, or to express a T-cell receptor comprising at least one of the amino acid sequences set forth in Table 6.

13. The modified T-cell of claim 12, wherein the one or more gene is selected from 4-1BB, PD-1, CD103 or TIM3.

14. The modified T-cell of claim 12 or 13, wherein the T-cell is a tissue-resident memory cell (TRM) or a CD8+ T-cell.

15. The modified T-cell of any one of claims 12 to 14, wherein the T-cell the T-cells are autologous to the subject being treated.

16. The modified T-cell of any one of claims 12 to 15, wherein baseline expression is normalized mean gene expression.

17. The modified T-cell of claim 16, wherein higher than baseline expression is at least about a 2-fold increase in expression relative to baseline expression and/or lower than baseline expression is at least about a 2-fold decrease in expression relative to baseline expression.

18. The modified T-cell of any one of claims 12 to 17, wherein the modified T-cell is genetically modified, optionally using one or more of gene editing, recombinant methods and/or a CRISPR/Cas system.

19. The modified T-cell of any one of claims 12 to 18, further modified to express a protein that binds to a cytokine, chemokine, lymphokine, or a receptor each thereof.

20. The modified T-cell of claim 19, wherein the protein comprises an antibody or an antigen binding fragment thereof.

21. The modified T-cell of claim 20, wherein the antibody is an IgG, IgA, IgM, IgE or IgD, or a subclass thereof.

22. The modified T-cell of claim 21, wherein the antibody is an IgG selected from the group of IgG1, IgG2, IgG3 or IgG4.

23. The modified T-cell of any one of claims 20 to 22, wherein the antigen binding fragment is selected from the group of a Fab, Fab′, F(ab′)2, Fv, Fd, single-chain Fvs (scFv), disulfide-linked Fvs (sdFv) or V_{L} or V_{H}.

24. The modified T-cell of any one of claims 12 to 23, wherein the modified T-cell comprises a chimeric antigen receptor (CAR).

25. The modified T-cell of claim 24, wherein the chimeric antigen receptor (CAR) comprises: (a) an antigen binding domain; (b) a hinge domain; (c) a transmembrane domain; (d) and an intracellular domain.

26. The modified T-cell of claim 25, wherein the CAR further comprises one or more costimulatory signaling regions.

27. The modified T-cell of claim 26, wherein the antigen binding domain comprises an anti-CD19 antigen binding domain, the transmembrane domain comprises a CD28, CD28H (TMIGD2), AMICA1 or a CD8 α transmembrane domain, the one or more costimulatory regions selected from a CD28 costimulatory signaling region, a 4-1BB costimulatory signaling region, an ICOS costimulatory signaling region, an AMICA1 costimulatory signaling region, a CD28H (TMIGD2) costimulatory signaling region, and an OX40 costimulatory region or a CD3 zeta signaling domain.

28. The modified T-cell of claim 27, wherein the anti-CD19 binding domain comprises a single-chain variable fragment (scFv) that specifically recognizes a humanized anti-CD19 binding domain.

29. The modified T-cell of claim 27 or 28, wherein the anti-CD19 binding domain scFv of the CAR comprises a heavy chain variable region and a light chain variable region.

30. The modified T-cell of claim 29, wherein the anti-CD19 binding domain of the CAR further comprises a linker polypeptide located between the anti-CD19 binding domain scFv heavy chain variable region and the anti-CD19 binding domain scFv light chain variable region.

31. The modified T-cell of claim 30, wherein the linker polypeptide of the CAR comprises a polypeptide of the sequence (GGGGS)n wherein n is an integer from 1 to 6.

32. The modified T-cell of any one of claims 24 to 31, wherein the CAR further comprises a detectable marker attached to the CAR.

33. The modified T-cell of any one of claims 24 to 32, wherein the CAR further comprises a purification marker attached to the CAR.

34. The modified T-cell of any one of claims 24 to 33, wherein the modified T-cell comprises a polynucleotide encoding the CAR, and optionally, wherein the polynucleotide encodes and anti-CD19 binding domain.

35. The modified T-cell of claim 34, wherein the polynucleotide further comprises a promoter operatively linked to the polynucleotide to express the polynucleotide in the modified T-cell.

36. The modified T-cell of claim 34, wherein the polynucleotide further comprises a 2A self-cleaving peptide (T2A) encoding polynucleotide sequence located upstream of a polynucleotide encoding the anti-CD19 binding domain.

37. The modified T-cell of any one of claims 34 to 36, wherein the polynucleotide further comprises a polynucleotide encoding a signal peptide located upstream of a polynucleotide encoding the anti-CD19 binding domain.

38. The modified T-cell of any one of claims 33 to 37, wherein the polynucleotide further comprises a vector.

39. The modified T-cell of claim 38, wherein the vector is a plasmid.

40. The modified T-cell of claim 38, wherein the vector is a viral vector selected from the group of a retroviral vector, a lentiviral vector, an adenoviral vector, and an adeno-associated viral vector.

41. A composition comprising a population of modified T-cells according to any one of claims 12 to 40.

42. A method of treating cancer in a subject and/or eliciting an anti-tumor response comprising administering to the subject or contacting the tumor with an effective amount of the modified T-cells according to any one of claims 12 to 40 and/or the composition according to claim 40, thereby treating cancer and/or eliciting an anti-tumor response in the subject.

43. A method of diagnosing cancer, comprising contacting a sample isolated from the subject with an agent that detects the presence of one or more genes set forth in Table 1, Table 2, Table 3, Table 4, Table Sand/or Table 7 in the sample isolated from the subject, wherein the presence of the one or more genes at higher or lower than baseline expression levels is diagnostic of cancer.

44. A method of diagnosing cancer, comprising contacting tissue-resident memory cells (TRMs) or a cancer sample isolated from the subject with an antibody or agent that recognizes and binds CD8, an antibody or agent that recognizes and binds PD-1, an antibody or agent that recognizes and binds TIM3, an antibody or agent that recognizes and binds LAG3, an antibody or agent that recognizes and binds AMICA1, an antibody or agent that recognizes and binds CD28H (TMIGD2), and an antibody or agent that recognizes and binds CTLA4 to determine the frequency of CD8⁺PD1⁺, CD8⁺TIM3⁺, CD8⁺LAG3⁺, CD8⁺AMICA1⁺, CD8⁺CD28H⁺, CD8⁺CTLA4⁺, CD8⁺PD1⁺TIM3⁺, CD8⁺PD1⁺LAG3⁺, CD8⁺PD1⁺AMICA1⁺, CD8⁺PD1⁺CD28H⁺, CD8⁺PD1⁺CTLA4⁺, CD8⁺TIM3⁺LAG3⁺, CD8⁺TIM3⁺AMICA1⁺, CD8⁺TIM3⁺CD28H⁺, CD8⁺TIM3⁺CTLA4⁺′CD8⁺LAG3⁺CTLA4+, CD8⁺LAG3⁺AMICA1⁺, CD8⁺LAG3⁺CD28H⁺, CD8⁺PD1⁺TIM3⁺LAG3⁺, CD8⁺LAG3⁺PD1AMICA1⁺, CD8⁺LAG3⁺PD1⁺CD28H⁺, CD8^{+PD}1⁺LAG3⁺CTLA4⁺, CD8⁺PD1⁺TIM3⁺CTLA4⁺, CD8⁺PD1⁺TIM3⁺CTLA4⁺AMICA1⁺′, CD8⁺PD1⁺TIM3⁺CTLA4⁺CD28H⁺′ or CD8⁺PD1⁺TIM3⁺CTLA4⁺AMICA⁺CD28H⁺′ TRMs, wherein a high frequency of one or more of these TRMs is diagnostic of cancer.

45. A method of diagnosing cancer in a subject comprising contacting tissue-resident memory cells (TRMs) isolated from the subject or cancer sample isolated from the subject, with an antibody or agent that recognizes and binds one or more proteins encoded by a gene set forth in Table 1, Table 2, Table 3, Table 4, Table 5 and/or Table 7 and, optionally, an antibody or agent that recognizes and binds CD8, an antibody or agent that recognizes and binds PD-1, an antibody or agent that recognizes and binds TIM3, an antibody or agent that recognizes and binds LAG3, an antibody or agent that recognizes and binds CD28H (TMIGD2), an antibody or agent that recognizes and binds AMICA1, an antibody or agent that recognizes and binds KLF3, an antibody or agent that recognizes and binds S1PR5, an antibody or agent that recognizes and binds S1PR1, an antibody or agent that recognizes and binds KLF2 and an antibody or agent that recognizes and binds CTLA4 to determine the frequency of TRMs expressing these proteins, wherein a high frequency of TRMs expressing these proteins is diagnostic of cancer.

46. A method of determining the density of tissue-resident memory cells (TRMs) in a cancer, tumor, or sample thereof comprising measuring expression of one or more gene selected from the group of 4-1BB, PD-1, CD103 or TIM3 or genes set forth in Table 1, Table 2, Table 3, Table 4, Table 5 and/or Table 7 in the cancer, tumor, or sample thereof, wherein higher or lower than baseline expression indicates higher density of TRMs in the cancer, tumor, or sample thereof.

47. A method of determining prognosis of a subject having cancer comprising measuring the density of tissue-resident memory cells (TRM) in a sample isolated from the subject, wherein a high density of TRM indicates a more positive prognosis, e.g., an increased probability and/or duration of survival.

48. A method of determining prognosis of a subject having cancer comprising contacting tissue-resident memory cells (TRMs) isolated from the subject with an antibody or agent that recognizes and binds CD8, an antibody or agent that recognizes and binds PD-1, an antibody or agent that recognizes and binds TIM3, an antibody or agent that recognizes and binds LAG3, an antibody or agent that recognizes and binds AMICA1, an antibody or agent that recognizes and binds CD28H (TMIGD2), and an antibody or agent that recognizes and binds CTLA4 to determine the frequency of CD8⁺PD1⁺, CD8⁺TIM3⁺, CD8⁺LAG3⁺, CD8⁺AMICA1⁺, CD8⁺CD28H⁺, CD8⁺CTLA4⁺, CD8⁺PD1⁺TIM3⁺, CD8⁺PD1⁺LAG3⁺, CD8⁺PD1⁺AMICA1⁺, CD8⁺PD1⁺CD28H⁺, CD8⁺PD1⁺CTLA4⁺, CD8⁺TIM3⁺LAG3⁺, CD8⁺TIM3⁺AMICA1⁺, CD8⁺TIM3⁺CD28H⁺, CD8⁺TIM3⁺CTLA4⁺, CD8⁺LAG3⁺CTLA4⁺, CD8⁺LAG3⁺AMICA1⁺, CD8⁺LAG3⁺CD28H⁺, CD8⁺PD1⁺TIM3⁺LAG3⁺, CD8⁺LAG3⁺PD1⁺AMICA1⁺, CD8⁺LAG3⁺PD1⁺CD28H⁺, CD8⁺PD1⁺LAG3⁺CTLA4⁺, CD8^{+PD}1⁺TIM3⁺CTLA4⁺, CD8⁺PD1⁺TIM3⁺CTLA4⁺AMICA1⁺′, CD8⁺PD1⁺TIM3⁺CTLA4⁺CD28H⁺′ or CD8^{+PD}1⁺TIM3⁺CTLA4⁺AMICA⁺CD28H⁺′ TRMs, wherein a high frequency of one or more of these TRMs indicates a more positive prognosis, e.g., an increased probability and/or duration of survival.

49. A method of determining prognosis of a subject having cancer comprising contacting tissue-resident memory cells (TRMs) isolated from the subject with an antibody or agent that recognizes and binds one or more proteins encoded by a gene set forth in Table 1, Table 2, Table 3, Table 4, Table 5 and/or Table 7 and, optionally, an antibody or agent that recognizes and binds CD8, an antibody or agent that recognizes and binds PD-1, an antibody or agent that recognizes and binds TIM3, an antibody or agent that recognizes and binds LAG3, an antibody or agent that recognizes and binds CD28H (TMIGD2), an antibody or agent that recognizes and binds AMICA1, an antibody or agent that recognizes and binds KLF3, an antibody or agent that recognizes and binds S1PR5, an antibody or agent that recognizes and binds S1PR1, an antibody or agent that recognizes and binds KLF2 and an antibody or agent that recognizes and binds CTLA4 to determine the frequency of TRMs expressing these proteins, wherein a high frequency of TRMs expressing these proteins indicates a more positive prognosis, e.g., an increased probability and/or duration of survival.

50. A method of determining prognosis of a subject having cancer comprising contacting tissue-resident memory cells (TRMs) isolated from the subject with an antibody or agent that recognizes and binds CD103 to determine the frequency of CD103+ TRMs or an antibody that recognizes and binds a protein encoded by a gene set forth in Table 1, Table 2, Table 3, Table 4, Table 5 and/or Table 7 to determine the frequency of TRMs expressing the protein, wherein a high or low frequency of TRMs expressing the protein indicates a more positive prognosis, e.g., an increased probability and/or duration of survival.

51. A method of determining the responsiveness of a subject having cancer to immunotherapy comprising contacting tissue-resident memory cells (TRMs) isolated from the subject with an antibody or agent that recognizes and binds CD8, an antibody or agent that recognizes and binds PD-1, an antibody or agent that recognizes and binds TIM3, an antibody or agent that recognizes and binds LAG3, an antibody or agent that recognizes and binds AMICA1, an antibody or agent that recognizes and binds CD28H (TMIGD2), and an antibody or agent that recognizes and binds CTLA4 to determine the frequency of CD8⁺PD1⁺, CD8⁺TIM3⁺, CD8⁺LAG3⁺, CD8⁺AMICA1⁺, CD8⁺CD28H⁺, CD8⁺CTLA4⁺, CD8⁺PD1⁺TIM3⁺, CD8⁺PD1⁺LAG3⁺, CD8⁺PD1⁺AMICA1⁺, CD8⁺PD1⁺CD28H⁺, CD8⁺PD1⁺CTLA4⁺, CD8⁺TIM3⁺LAG3⁺, CD8⁺TIM3⁺AMICA1⁺, CD8⁺TIM3⁺CD28H⁺, CD8⁺TIM3⁺CTLA4⁺, CD8⁺LAG3⁺CTLA4⁺, CD8⁺LAG3⁺AMICA1⁺, CD8⁺LAG3⁺CD28H⁺, CD8⁺PD1⁺TIM3⁺LAG3⁺, CD8⁺LAG3⁺PD1⁺AMICA1⁺, CD8⁺LAG3⁺PD1⁺CD28H⁺, CD8⁺PD1⁺LAG3⁺CTLA4⁺, CD8⁺PD1⁺TIM3⁺CTLA4⁺, CD8⁺PD1⁺TIM3⁺CTLA4⁺AMICA1⁺′, CD8⁺PD1⁺TIM3⁺CTLA4⁺CD28H⁺′ or CD8⁺PD1⁺TIM3⁺CTLA4⁺AMICA⁺CD28H⁺, TRMs, wherein a high frequency of one or more of these TRMs indicates responsiveness to immunotherapy.

52. A method of determining the responsiveness of a subject having cancer to immunotherapy comprising contacting tissue-resident memory cells (TRMs) isolated from the subject with an antibody or agent that recognizes and binds one or more proteins encoded by a gene set forth in Table 1, Table 2, Table 3, Table 4, Table 5 and/or Table 7 and, optionally, an antibody or agent that recognizes and binds CD8, an antibody or agent that recognizes and binds PD-1, an antibody or agent that recognizes and binds TIM3, an antibody or agent that recognizes and binds LAG3, an antibody or agent that recognizes and binds CD28H (TMIGD2), an antibody or agent that recognizes and binds AMICA1,
an antibody or agent that recognizes and binds KLF3, an antibody or agent that recognizes and binds S1PR5, an antibody or agent that recognizes and binds S1PR1, an antibody or agent that recognizes and binds KLF2 and an antibody or agent that recognizes and binds CTLA4 to determine the frequency of TRMs expressing these proteins, wherein a high frequency of TRMs expressing these proteins indicates responsiveness to immunotherapy.

53. The method of any of claims 43 to 52, wherein the TRMs are CD19-CD20-CD14-CD56-CD4-CD45⁺CD3⁺CD8+ T-cells.

54. A method of determining prognosis of a subject having cancer comprising measuring the density of CD103 or proteins encoded by one or more gene set forth in Table 1, Table 2, Table 3, Table 4, Table 5 and/or Table 7 in a sample isolated from the subject, wherein a high or low density of proteins indicates a more positive prognosis, e.g., an increased probability and/or duration of survival.

55. A method of identifying a subject that will or is likely to respond to a cancer therapy, comprising contacting a sample isolated from the subject with an agent that detects the presence of one or more genes set forth in Table 1, Table 2, Table 3, Table 4, Table 5 and/or Table 7 in the sample, wherein the presence of the one or more genes at higher or lower than baseline expression levels indicates that the subject is likely to respond to cancer therapy.

56. The method of any one of claim 43, 46 or 55, wherein baseline expression is normalized mean gene expression.

57. The method of claim 56, wherein higher than baseline expression is at least about a 2-fold increase in expression relative to baseline expression and/or lower than baseline expression is at least about a 2-fold decrease in expression relative to baseline expression.

58. The method of any one of claims 43 to 57, further comprising administering a cancer therapy to the subject.

59. The method of claim 58, wherein the cancer therapy is chemotherapy, immunotherapy, radiation therapy, and/or administering to the subject or contacting the tumor with an effective amount of the modified T-cells according to any one of claims 12 to 40 and/or the composition according to claim 40.

60. The method of any one of claims 43 to 59, wherein the cancer, tumor, or sample is contacted with an agent, optionally including a detectable label or tag.

61. The method of claim 60, wherein the detectable label or tag comprises a radioisotope, a metal, horseradish peroxidase, alkaline phosphatase, avidin or biotin.

62. The method of claim 60 or 61, wherein the agent comprises a polypeptide that binds to an expression product encoded by the gene, or a polynucleotide that hybridizes to a nucleic acid sequence encoding all or a portion of the gene.

63. The method of claim 62, wherein the polypeptide comprises an antibody, an antigen binding fragment thereof, or a receptor that binds to the gene.

64. The method of claim 63, wherein the antibody is an IgG, IgA, IgM, IgE or IgD, or a subclass thereof.

65. The method of claim 64, wherein the IgG is an IgG1, IgG2, IgG3 or IgG4.

66. The method of any one of claims 63 to 65 wherein the antigen binding fragment is a Fab, Fab′, F(ab′)2, Fv, Fd, single-chain Fvs (scFv), disulfide-linked Fvs (sdFv) or VL or VH.

67. The method of any one of claims 43 to 66, wherein the agent is contacted with the cancer, tumor, or sample in conditions under which it can bind to the gene it targets.

68. The method of any one of claims 43 to 67, wherein the method comprises detection by immunohistochemistry (IHC), in-situ hybridization (ISH), ELISA, immunoprecipitation, immunofluorescence, chemiluminescence, radioactivity, X-ray, nucleic acid hybridization, protein-protein interaction, immunoprecipitation, flow cytometry, Western blotting, polymerase chain reaction, DNA transcription, Northern blotting and/or Southern blotting.

69. The method of any one of claims 43 to 68, wherein the sample comprises cells, tissue, an organ biopsy, an epithelial tissue, a lung, respiratory or airway tissue or organ, a circulatory tissue or organ, a skin tissue, bone tissue, muscle tissue, head, neck, brain, skin, bone and/or blood sample.

70. The method of any one of claims 1 to 11 or claims 41 to 69, wherein the cancer or tumor is an epithelial, a head, neck, lung, lung, prostate, colon, pancreas, esophagus, liver, skin, kidney, adrenal gland, brain, or comprises a lymphoma, breast, endometrium, uterus, ovary, testes, lung, prostate, colon, pancreas, esophagus, liver, skin, kidney, adrenal gland and/or brain cancer or tumor, a metastasis or recurring tumor, cancer or neoplasia, a non-small cell lung cancer (NSCLC) and/or head and neck squamous cell cancer (HNSCC).

71. The method of any one of claims 43 to 70, wherein the method comprises detecting in the subject, the cells or the sample the number or density of Trm cells that are CD19−CD20−CD14−CD56−CD4−CD45+CD3+CD8+ T-cells.

72. A kit comprising one or more of the modified T-cells according to any one of claims 12 to 40 and/or the composition according to claim 41 and instructions for use.

73. The kit of claim 72, wherein the instruction for use provide directions to conduct the method of any one of claims 1 to 11 and/or 42 to 71.
